(19) United States
(12) Patent Application Publication
(10) Pub. No.: US 2023/0295166 A1
(43) Pub. Date: Sep. 21, 2023

(54) ATR INHIBITORS AND USES THEREOF

(71) Applicant: ANTENGENE DISCOVERY LIMITED, Shanghai (CN)

(72) Inventors: Bo SHAN, Shanghai (CN); Bing HOU, Shanghai (CN); Hui YUWEN, Shanghai (CN); Peng CHEN, Shanghai (CN); Zhongyang SHI, Shanghai (CN); Zhengsong GU, Shanghai (CN); Ping CHEN, Shanghai (CN); Zhenwei CAI, Shanghai (CN); Jay MEI, Shanghai (CN)

(21) Appl. No.: 18/040,824

(22) PCT Filed: Aug. 6, 2021
(86) PCT No.: PCT/CN2021/111278

§ 371 Date: Feb. 7, 2023

(30) Foreign Application Priority Data
Aug. 7, 2020 (WO) PCT/CN2020/107884
Aug. 19, 2020 (WO) PCT/CN2020/110005
Apr. 2, 2021 (WO) PCT/CN2021/085190
Jul. 12, 2021 (WO) PCT/CN2021/105708

(51) Int. Cl. C07D 487/04; C07D 513/04; C07D 519/00; C07F 9/6561; C07D 413/14; A61P 35/00; C07D 498/04

(52) U.S. Cl. CPC C07D 487/04; C07D 513/04; C07D 519/00; C07F 9/6561; C07D 413/14; A61P 35/00; C07D 498/04

(57) ABSTRACT

The present disclosure relates to novel compounds useful as inhibitors of ATR kinase, as well as pharmaceutical compositions comprising these compounds and methods of treatment by administration of these compounds or the pharmaceutical compositions.

### FIELD OF THE DISCLOSURE

The present disclosure generally relates to novel compounds useful as ATR inhibitors, as well as pharmaceutical compositions comprising these compounds and methods of treatment by administration of these compounds or the pharmaceutical compositions.

### BACKGROUND OF THE DISCLOSURE

ATR (also known as FRAP-Related Protein 1; FRP1, MEC1, SCKL, SECKL1) protein kinase is a member of the PI3-Kinase like kinase (PIKK) family of proteins involved in repair and maintenance of the genome and its stability. It is essential to the viability of replicating cells and is activated during S-phase to regulate firing of replication origins and to repair damaged replication forks. Therefore, ATR inhibitors have the potential to be an efficient way in cancer treatment.

While progress has been made for ATR inhibitors, there is still a strong need in the art to develop improved pharmaceutics having inhibitory activity against ATR.

### SUMMARY OF THE DISCLOSURE

The present disclosure provides compounds, including stereoisomers, pharmaceutically acceptable salts, tautomers and prodrugs thereof, which are capable of inhibiting ATR protein kinase. Methods for use of such compounds for treatment of various diseases or conditions, such as cancer, are also provided.

In one aspect, the present disclosure provides a compound having Formula

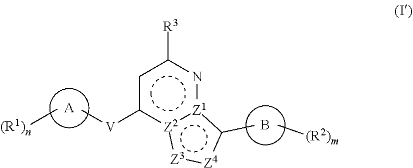

or a pharmaceutically acceptable salt thereof,
wherein
Z¹ is C or N;
Z² is C or N;
Z³ is CR^{d}, N, O, S, S(O) or S(O)₂;
Z⁴ is CH or N;
V is a direct bond, or alkyl optionally substituted with one or more R^{e} or —N(R^{a})—;
Ring A is absent, 3- to 6-membered cycloalkyl, 5- to 6-membered heterocyclyl, 5- to 6-membered aryl, or 5- to 6-membered heteroaryl;
R¹, in each occurrence, is selected from the group consisting of hydrogen, halogen, hydroxyl, cyano, alkyl, haloalkyl, hydroxylalkyl, —C(O)N(R^{a})₂, —C(O)OR^{a}, —S(O)₂(R^{b}), —S(O)(NH)(R^{b}) and —P(O)(R^{b})₂;
Ring B is 5- to 6-membered heterocyclyl or 5- to 6-membered heteroaryl;
R², in each occurrence, is halogen, alkyl, haloalkyl, or cycloalkyl;
R³ is;

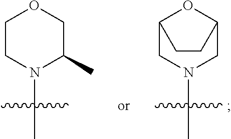

R^{a} and R^{d} are each independently hydrogen, halogen or alkyl;
R^{b} is alkyl, 3- to 6-membered cycloalkyl, 5- to 6-membered heterocyclyl, 5- to 6-membered aryl, or 5- to 6-membered heteroaryl, wherein said cycloalkyl, heterocyclyl, and heteroaryl are optionally substituted with one or more R^{c};
R^{c} is selected from the group consisting of hydroxyl, halogen, cyano, amino, alkyl, alkoxyl, and haloalkyl;
R^{e} is hydroxyl, halogen or alkyl;
n is 0, 1, 2, or 3; and
m is 0, 1, 2 or 3.

In one aspect, the present disclosure provides a compound having Formula (I):

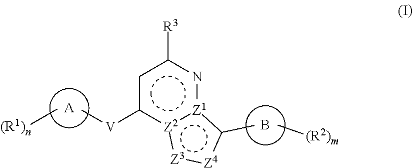

or a pharmaceutically acceptable salt thereof,
wherein
Z¹ is C or N;
Z² is C or N;
Z³ is CH, N, or S;
Z⁴ is CH or N;
V is a direct bond or —N(R^{a})—;
Ring A is absent, 3- to 6-membered cycloalkyl, 5- to 6-membered heterocyclyl, 5- to 6-membered aryl, or 5- to 6-membered heteroaryl;
R¹ is hydrogen, halogen, alkyl, —S(O)₂(R^{b}), or —S(O)(NH)(R^{b});
Ring B is 5- to 6-membered heterocyclyl or 5- to 6-membered heteroaryl;
R² is halogen, alkyl, haloalkyl, or cycloalkyl;
R³ is

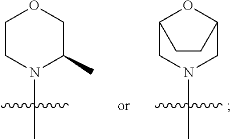

R^{a} is hydrogen or alkyl;
R^{b} is alkyl, 3- to 6-membered cycloalkyl, 5- to 6-membered heterocyclyl, 5- to 6-membered aryl, or 5- to 6-membered heteroaryl, wherein said cycloalkyl, heterocyclyl, and heteroaryl are optionally substituted with one or more R^{c};
R^{c} is selected from the group consisting of hydroxyl, halogen, cyano, amino, alkyl, alkoxyl, and haloalkyl;
n is 0, 1, 2, or 3; and
m is 0, 1, 2 or 3.

In some embodiments, the present disclosure provides compound having a formula selected from the group consisting of:

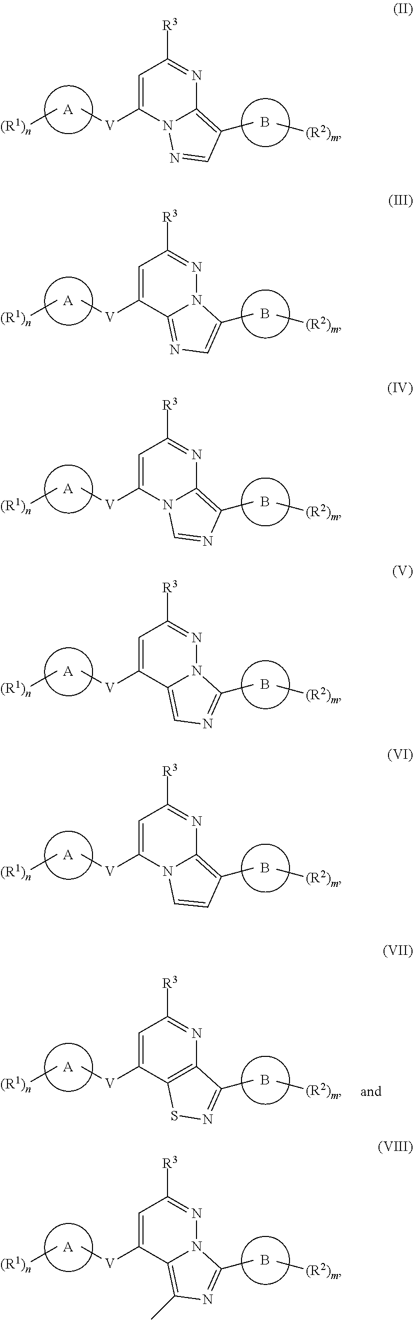

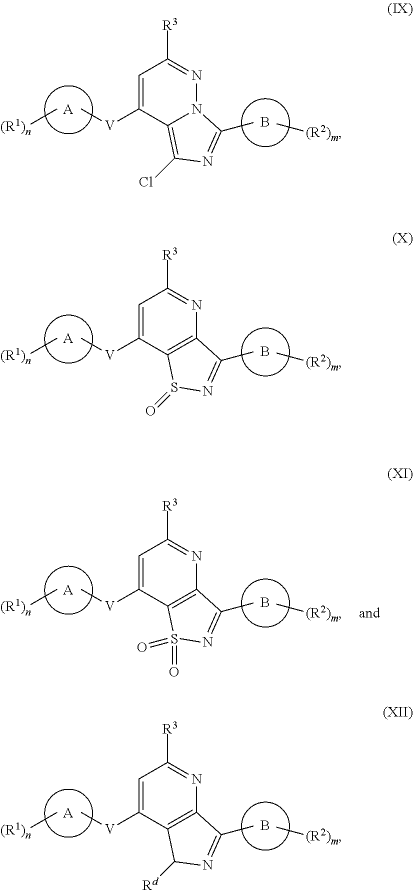

or a pharmaceutically acceptable salt thereof.

In some embodiments, the present disclosure provides compound having a formula selected from the group consisting of:

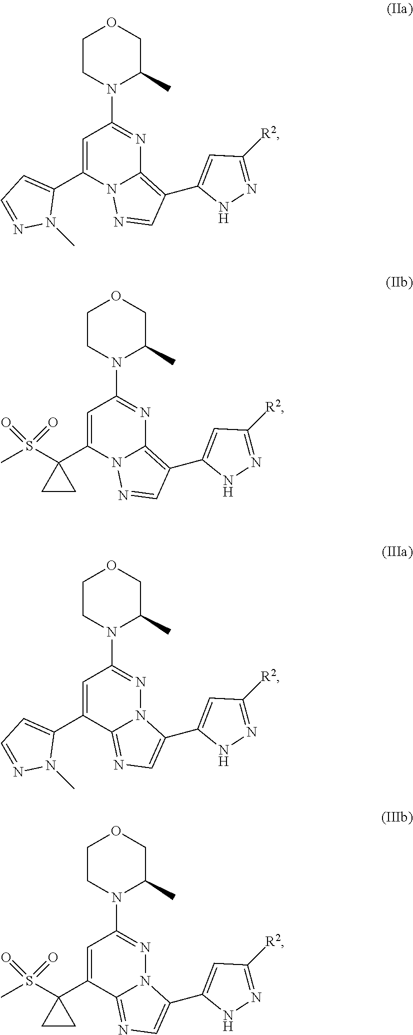

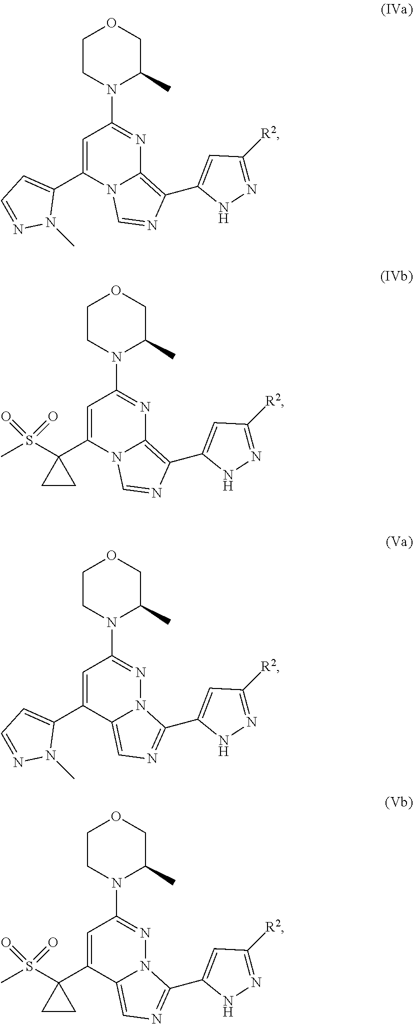

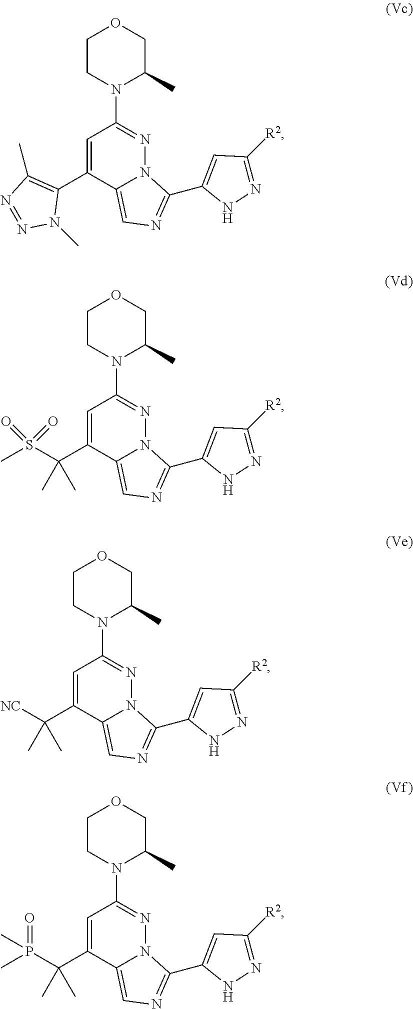

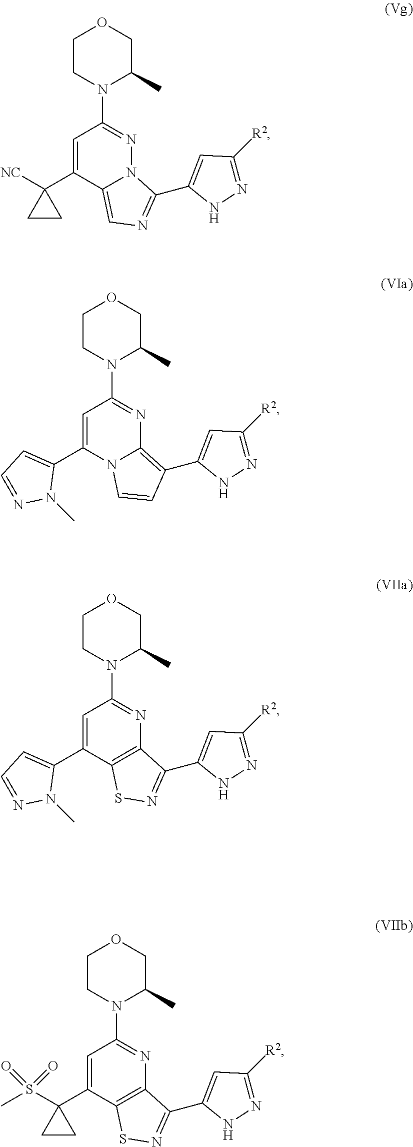

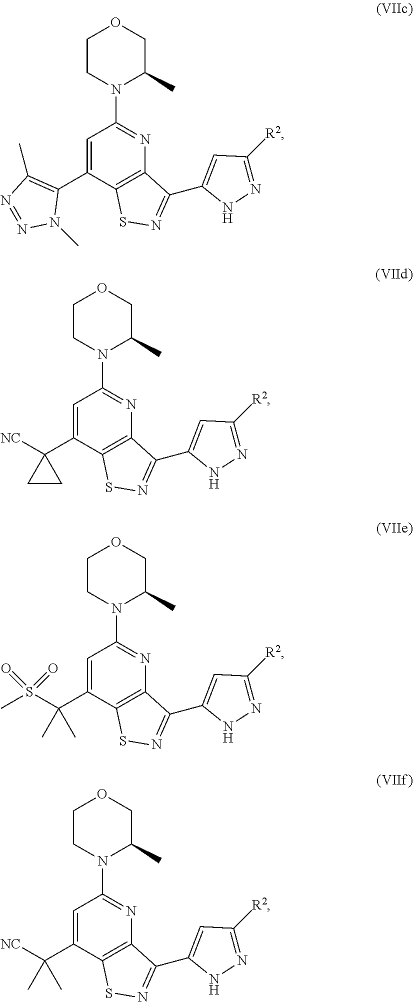

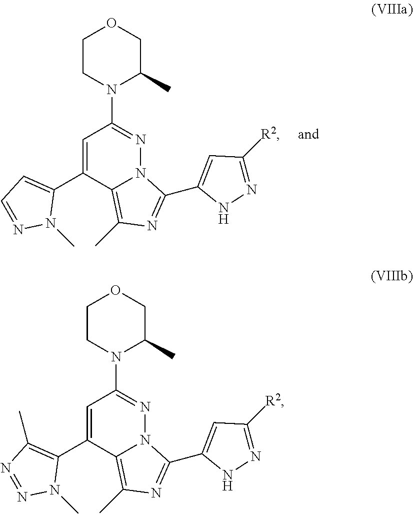

or a pharmaceutically acceptable salt thereof.

In some embodiments, the present disclosure provides a compound having a formula selected from the group consisting of:

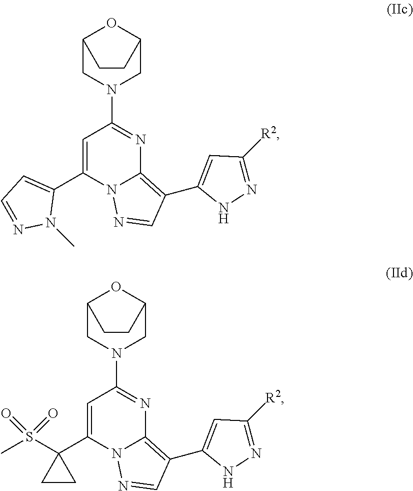

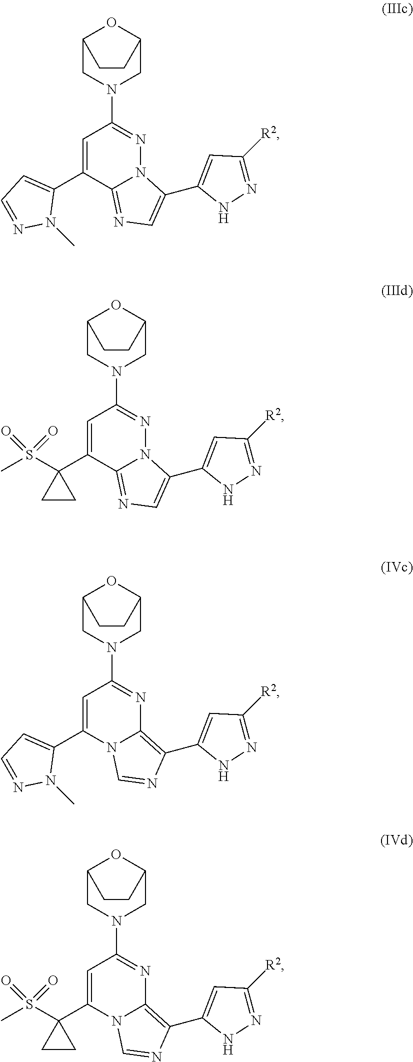

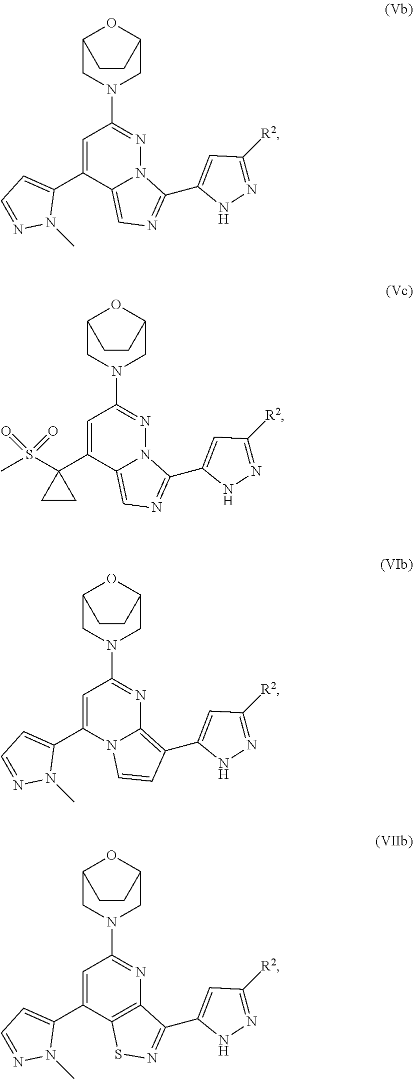

or a pharmaceutically acceptable salt thereof.

In another aspect, the present disclosure provides a pharmaceutical composition comprising the compound of the present disclosure or a pharmaceutically acceptable salt thereof, and a pharmaceutically acceptable excipient.

In a further aspect, the present disclosure provides a method for treating cancer, comprising administering an effective amount of a compound of the present disclosure or a pharmaceutically acceptable salt thereof or the pharmaceutical composition of the present disclosure to a subject in need thereof.

In a further aspect, the present disclosure provides use of the compound of the present disclosure or a pharmaceutically acceptable salt thereof or the pharmaceutical composition of the present disclosure in the manufacture of a medicament in the prevention or treatment of cancer.

In a further aspect, the present disclosure provides compounds of the present disclosure or a pharmaceutically acceptable salt thereof or the pharmaceutical composition of the present disclosure, for use in the treatment of cancer.

In a further aspect, the present disclosure provides a method for inhibiting ATR kinase in a subject in need thereof, comprising administering an effective amount of a compound of the present disclosure or a pharmaceutically acceptable salt thereof or the pharmaceutical composition of the present disclosure to the subject.

### DETAILED DESCRIPTION OF THE DISCLOSURE

Reference will now be made in detail to certain embodiments of the present disclosure, examples of which are illustrated in the accompanying structures and formulas. While the present disclosure will be described in conjunction with the enumerated embodiments, it will be understood that they are not intended to limit the present disclosure to those embodiments. On the contrary, the present disclosure is intended to cover all alternatives, modifications, and equivalents, which may be included within the scope of the present disclosure as defined by the claims. One skilled in the art will recognize many methods and materials similar or equivalent to those described herein, which could be used in the practice of the present disclosure. The present disclosure is in no way limited to the methods and materials described. In the event that one or more of the incorporated references and similar materials differs from or contradicts this application, including but not limited to defined terms, term usage, described techniques, or the like, the present disclosure controls. All references, patents, patent applications cited in the present disclosure are hereby incorporated by reference in their entireties.

It is appreciated that certain features of the present disclosure, which are, for clarity, described in the context of separate embodiments, can also be provided in combination in a single embodiment. Conversely, various features of the present disclosure, which are, for brevity, described in the context of a single embodiment, can also be provided separately or in any suitable sub-combination. It must be noted that, as used in the specification and the appended claims, the singular forms “a,” “an,” and “the” include plural forms of the same unless the context clearly dictates otherwise. Thus, for example, reference to “a compound” includes a plurality of compounds.

### Definitions

Definitions of specific functional groups and chemical terms are described in more detail below. For purposes of this disclosure, the chemical elements are identified in accordance with the Periodic Table of the Elements, CAS version, Handbook of Chemistry and Physics, 75^{th}Ed., inside cover, and specific functional groups are generally defined as described therein. Additionally, general principles of organic chemistry, as well as specific functional moieties and reactivity, are described in Organic Chemistry, Thomas Sorrell, 2^{nd} Edition, University Science Books, Sausalito, 2006; Smith and March March's Advanced Organic Chemistry, 6^{th} Edition, John Wiley & Sons, Inc., New York, 2007; Larock, Comprehensive Organic Transformations, 3^{rd} Edition, VCH Publishers, Inc., New York, 2018; Carruthers, Some Modern Methods of Organic Synthesis, 4^{th} Edition, Cambridge University Press, Cambridge, 2004; the entire contents of each of which are incorporated herein by reference.

Generally, the nomenclature used herein and the laboratory procedures in organic chemistry, medicinal chemistry, and pharmacology described herein are those well known and commonly employed in the art. Unless defined otherwise, all technical and scientific terms used herein generally have the same meaning as commonly understood by one of ordinary skill in the art to which this disclosure belongs.

It should be noted that if there is a discrepancy between a depicted structure and a name given that structure, the depicted structure is to be accorded more weight. In addition, if the stereochemistry of a structure or a portion of a structure is not indicated with, for example, bold or dashed lines, the structure or portion of the structure is to be interpreted as encompassing all stereoisomers of it.

At various places in the present disclosure, linking substituents are described. Where the structure clearly requires a linking group, the Markush variables listed for that group are understood to be linking groups. For example, if the structure requires a linking group and the Markush group definition for that variable lists “alkyl”, then it is understood that the “alkyl” represents a linking alkylene group.

When a bond to a substituent is shown to cross a bond connecting two atoms in a ring, then such substituent may be bonded to any atom in the ring. When a substituent is listed without indicating the atom via which such substituent is bonded to the rest of the compound of a given formula, then such substituent may be bonded via any atom in such formula. Combinations of substituents and/or variables are permissible, but only if such combinations result in stable compounds.

When any variable (e.g., Rⁱ) occurs more than one time in any constituent or formula for a compound, its definition at each occurrence is independent of its definition at every other occurrence. Thus, for example, if a group is shown to be substituted with 0-2 Rⁱ moieties, then the group may optionally be substituted with up to two Rⁱ moieties and Rⁱ at each occurrence is selected independently from the definition of Rⁱ. Also, combinations of substituents and/or variables are permissible, but only if such combinations result in stable compounds.

As used herein, the term “Cᵢ₋ⱼ” indicates a range of the carbon atoms numbers, wherein i and j are integers and the range of the carbon atoms numbers includes the endpoints (i.e. i and j) and each integer point in between, and wherein j is greater than i. For examples, C₁₋₆ indicates a range of one to six carbon atoms, including one carbon atom, two carbon atoms, three carbon atoms, four carbon atoms, five carbon atoms and six carbon atoms. In some embodiments, the term “C₁₋₁₂” indicates 1 to 12, particularly 1 to 10, particularly 1 to 8, particularly 1 to 6, particularly 1 to 5, particularly 1 to 4, particularly 1 to 3 or particularly 1 to 2 carbon atoms.

As used herein, the term “alkyl”, whether as part of another term or used independently, refers to a saturated linear or branched-chain hydrocarbon radical, which may be optionally substituted independently with one or more substituents described below. The term “Cᵢ₋ⱼ alkyl” refers to an alkyl having i to j carbon atoms.

In some embodiments, alkyl groups contain 1 to 10 carbon atoms. In some embodiments, alkyl groups contain 1 to 9 carbon atoms. In some embodiments, alkyl groups contain 1 to 8 carbon atoms, 1 to 7 carbon atoms, 1 to 6 carbon atoms, 1 to 5 carbon atoms, 1 to 4 carbon atoms, 1 to 3 carbon atoms, or 1 to 2 carbon atoms.

Examples of “C₁₋₁₀ alkyl” include, but are not limited to, methyl, ethyl, propyl, butyl, pentyl, hexyl, heptyl, octyl, nonyl, and decyl. Examples of “C₁₋₆ alkyl” are methyl, ethyl, propyl, isopropyl, n-butyl, i-butyl, s-butyl, t-butyl, n-pentyl, 2-pentyl, 3-pentyl, 2-methyl-2-butyl, 3-methyl-2-butyl, 3-methyl-1-butyl, 2-methyl-1-butyl, 1-hexyl, 2-hexyl, 3-hexyl, 2-methyl-2-pentyl, 3-methyl-2-pentyl, 4-methyl-2-pentyl, 3-methyl-3-pentyl, 2-methyl-3-pentyl, 2,3-dimethyl-2-butyl, 3,3-dimethyl-2-butyl, and the like.

As used herein, the term “alkoxyl”, whether as part of another term or used independently, refers to an alkyl group, as previously defined, attached to the parent molecule through an oxygen atom. The term “Cᵢ₋ⱼ alkoxy” means that the alkyl moiety of the alkoxy group has i to j carbon atoms. In some embodiments, alkoxy groups contain 1 to 10 carbon atoms. In some embodiments, alkoxy groups contain 1 to 9 carbon atoms. In some embodiments, alkoxy groups contain 1 to 8 carbon atoms, 1 to 7 carbon atoms, 1 to 6 carbon atoms, 1 to 5 carbon atoms, 1 to 4 carbon atoms, 1 to 3 carbon atoms, or 1 to 2 carbon atoms. Examples of “C₁₋₆ alkoxyl” include, but are not limited to, methoxy, ethoxy, propoxy (e.g. n-propoxy and isopropoxy), t-butoxy, neopentoxy, n-hexoxy, and the like.

As used herein, the term “amino” refers to —NH₂. Amino groups may also be substituted with one or more groups such as alkyl, aryl, carbonyl or other amino groups.

As used herein, the term “aryl”, whether as part of another term or used independently, refers to monocyclic and polycyclic ring systems having a total of 5 to 20 ring members, wherein at least one ring in the system is aromatic and wherein each ring in the system contains 3 to 12 ring members. Examples of “aryl” include, but are not limited to, phenyl, biphenyl, naphthyl, anthracyl and the like, which may bear one or more substituents. Also included within the scope of the term “aryl”, as it is used herein, is a group in which an aromatic ring is fused to one or more additional rings. In the case of polycyclic ring system, only one of the rings needs to be aromatic (e.g., 2,3-dihydroindole), although all of the rings may be aromatic (e.g., quinoline). The second ring can also be fused or bridged. Examples of polycyclic aryl include, but are not limited to, benzofuranyl, indanyl, phthalimidyl, naphthimidyl, phenanthridinyl, or tetrahydronaphthyl, and the like. Aryl groups can be substituted at one or more ring positions with substituents as described above.

As used herein, the term “cycloalkyl”, whether as part of another term or used independently, refer to a monovalent non-aromatic, saturated or partially unsaturated monocyclic and polycyclic ring system, in which all the ring atoms are carbon and which contains at least three ring forming carbon atoms. In some embodiments, the cycloalkyl may contain 3 to 12 ring forming carbon atoms, 3 to 10 ring forming carbon atoms, 3 to 9 ring forming carbon atoms, 3 to 8 ring forming carbon atoms, 3 to 7 ring forming carbon atoms, 3 to 6 ring forming carbon atoms, 3 to 5 ring forming carbon atoms, 4 to 12 ring forming carbon atoms, 4 to 10 ring forming carbon atoms, 4 to 9 ring forming carbon atoms, 4 to 8 ring forming carbon atoms, 4 to 7 ring forming carbon atoms, 4 to 6 ring forming carbon atoms, 4 to 5 ring forming carbon atoms. Cycloalkyl groups may be saturated or partially unsaturated.

Cycloalkyl groups may be substituted. In some embodiments, the cycloalkyl group may be a saturated cyclic alkyl group. In some embodiments, the cycloalkyl group may be a partially unsaturated cyclic alkyl group that contains at least one double bond or triple bond in its ring system. In some embodiments, the cycloalkyl group may be monocyclic or polycyclic. Examples of monocyclic cycloalkyl group include, but are not limited to, cyclopropyl, cyclobutyl, cyclopentyl, 1-cyclopent-1-enyl, 1-cyclopent-2-enyl, 1-cyclopent-3-enyl, cyclohexyl, 1-cyclohex-1-enyl, 1-cyclohex-2-enyl, 1-cyclohex-3-enyl, cyclohexadienyl, cycloheptyl, cyclooctyl, cyclononyl, cyclodecyl, cycloundecyl and cyclododecyl. Examples of polycyclic cycloalkyl group include, but are not limited to, adamantyl, norbornyl, fluorenyl, spiro-pentadienyl, spiro[3.6]-decanyl, bicyclo[1,1,1]pentenyl, bicyclo[2,2,1]heptenyl, and the like.

As used herein, the term “cyano” refers to —CN.

As used herein, the term “halogen” refers to an atom selected from fluorine (or fluoro), chlorine (or chloro), bromine (or bromo) and iodine (or iodo).

As used herein, the term “haloalkyl” refers to an alkyl, as defined above, that is substituted by one or more halogens, as defined above. Examples of haloalkyl include, but are not limited to, trifluoromethyl, difluoromethyl, trichloromethyl, 2,2,2-trifluoroethyl, 1,2-difluoroethyl, 3-bromo-2-fluoropropyl, 1,2-dibromoethyl, and the like.

As used herein, the term “heteroatom” refers to nitrogen, oxygen, sulfur or phosphorus, and includes any oxidized form of nitrogen or sulfur, and any quaternized form of a basic nitrogen (including N-oxides).

As used herein, the term “heteroaryl”, whether as part of another term or used independently, refers to an aryl group having, in addition to carbon atoms, one or more heteroatoms. The heteroaryl group can be monocyclic. Examples of monocyclic heteroaryl include, but are not limited to, thienyl, furanyl, pyrrolyl, imidazolyl, pyrazolyl, triazolyl, tetrazolyl, oxazolyl, isoxazolyl, oxadiazolyl, thiazolyl, isothiazolyl, thiadiazolyl, pyridyl, pyridazinyl, pyrimidinyl, pyrazinyl, indolizinyl, purinyl, naphthyridinyl, benzofuranyl and pteridinyl. The heteroaryl group also includes polycyclic groups in which a heteroaromatic ring is fused to one or more aryl, cycloaliphatic, or heterocyclyl rings, where the radical or point of attachment is on the heteroaromatic ring. Examples of polycyclic heteroaryl include, but are not limited to, indolyl, isoindolyl, benzothienyl, benzofuranyl, benzo[1,3]dioxolyl, dibenzofuranyl, indazolyl, benzimidazolyl, benzthiazolyl, quinolyl, isoquinolyl, dihydroquinolinyl, dihydroisoquinolinyl, tetrahydroquinolinyl, tetrahydroisoquinolinyl, cinnolinyl, phthalazinyl, quinazolinyl, quinoxalinyl, 4H-quinolizinyl, carbazolyl, acridinyl, phenazinyl, phenothiazinyl, phenoxazinyl, tetrahydroquinolinyl, tetrahydroisoquinolinyl, and the like.

As used herein, the term “heterocyclyl” refers to a saturated or partially unsaturated carbocyclyl group in which one or more ring atoms are heteroatoms independently selected from oxygen, sulfur, nitrogen, phosphorus, and the like, the remaining ring atoms being carbon, wherein one or more ring atoms may be optionally substituted independently with one or more substituents. In some embodiments, the heterocyclyl is a saturated heterocyclyl. In some embodiments, the heterocyclyl is a partially unsaturated heterocyclyl having one or more double bonds in its ring system. In some embodiments, the heterocyclyl may contains any oxidized form of carbon, nitrogen or sulfur, and any quaternized form of a basic nitrogen. “Heterocyclyl” also includes radicals wherein the heterocyclyl radicals are fused with a saturated, partially unsaturated, or fully unsaturated (i.e., aromatic) carbocyclic or heterocyclic ring. The heterocyclyl radical may be carbon linked or nitrogen linked where such is possible. In some embodiments, the heterocycle is carbon linked. In some embodiments, the heterocycle is nitrogen linked. For example, a group derived from pyrrole may be pyrrol-1-yl (nitrogen linked) or pyrrol-3-yl (carbon linked). Further, a group derived from imidazole may be imidazol-1-yl (nitrogen linked) or imidazol-3-yl (carbon linked).

In some embodiments, the term “3- to 12-membered heterocyclyl” refers to a 3- to 12-membered saturated or partially unsaturated monocyclic or polycyclic heterocyclic ring system having 1 to 3 heteroatoms independently selected from nitrogen, oxygen, or sulfur. The fused, spiro and bridged ring systems are also included within the scope of this definition. Examples of monocyclic heterocyclyl include, but are not limited to oxetanyl, 1,1-dioxothietanylpyrrolidyl, tetrahydrofuryl, tetrahydrothienyl, pyrrolyl, furanyl, thienyl, pyrazolyl, imidazolyl, triazolyl, oxazolyl, thiazolyl, piperidyl, piperazinyl, piperidinyl, morpholinyl, pyridinyl, pyrazinyl, pyrimidinyl, pyridazinyl, triazinyl, pyridonyl, pyrimidonyl, pyrazinonyl, pyrimidonyl, pyridazonyl, pyrrolidinyl, triazinonyl, and the like. Examples of fused heterocyclyl include, but are not limited to, phenyl fused ring or pyridinyl fused ring, such as quinolinyl, isoquinolinyl, tetrahydroquinolinyl, tetrahydroisoquinolinyl, quinoxalinyl, quinolizinyl, quinazolinyl, azaindolizinyl, pteridinyl, chromenyl, isochromenyl, indolyl, isoindolyl, indolizinyl, indazolyl, purinyl, benzofuranyl, isobenzofuranyl, benzimidazolyl, benzothienyl, benzothiazolyl, carbazolyl, phenazinyl, phenothiazinyl, phenanthridinyl, imidazo[1,2-a]pyridinyl, [1,2,4]triazolo[4,3-a]pyridinyl, triazolo[4,3-a]pyridinyl groups, and the like. Examples of spiro heterocyclyl include, but are not limited to, spiropyranyl, spirooxazinyl, and the like. Examples of bridged heterocyclyl include, but are not limited to, morphanyl, hexamethylenetetraminyl, 3-aza-bicyclo[3.1.0]hexane, 8-aza-bicyclo[3.2.1]octane, 1-aza-bicyclo[2.2.2]octane, 1,4-diazabicyclo[2.2.2]octane (DABCO), and the like.

As used herein, the term “hydroxyl” refers to —OH.

As used herein, the term “partially unsaturated” refers to a radical that includes at least one double or triple bond. The term “partially unsaturated” is intended to encompass rings having multiple sites of unsaturation, but is not intended to include aromatic (i.e., fully unsaturated) moieties.

As used herein, the term “substituted”, whether preceded by the term “optionally” or not, means that one or more hydrogens of the designated moiety are replaced with a suitable substituent. It will be understood that “substitution” or “substituted with” includes the implicit proviso that such substitution is in accordance with permitted valence of the substituted atom and that the substitution results in a stable or chemically feasible compound, e.g., which does not spontaneously undergo transformation such as by rearrangement, cyclization, elimination, etc. Unless otherwise indicated, an “optionally substituted” group may have a suitable substituent at each substitutable position of the group, and when more than one position in any given structure may be substituted with more than one substituent selected from a specified group, the substituent may be either the same or different at every position.

It will be understood by those skilled in the art that substituents can themselves be substituted, if appropriate. Unless specifically stated as “unsubstituted”, references to chemical moieties herein are understood to include substituted variants. For example, reference to an “aryl” group or moiety implicitly includes both substituted and unsubstituted variants.

### Compounds

The present disclosure provides novel compounds of Formula (I) and pharmaceutically acceptable salts thereof, synthetic methods for making the compounds, pharmaceutical compositions containing them and various uses of the disclosed compounds.

In one aspect, the present disclosure provides a compound having Formula (I′):

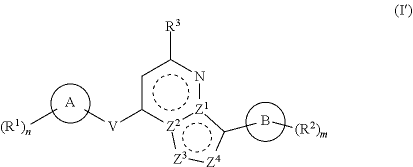

or a pharmaceutically acceptable salt thereof,
wherein
Z¹ is C or N;
Z² is C or N;
Z³ is CR^{d}, N, O, S, S(O) or S(O)₂;
Z⁴ is CH or N;
V is a direct bond, or alkyl optionally substituted with one or more R or —N(R^{a})—;
Ring A is absent, 3- to 6-membered cycloalkyl, 5- to 6-membered heterocyclyl, 5- to 6-membered aryl, or 5- to 6-membered heteroaryl;
R¹, in each occurrence, is selected from the group consisting of hydrogen, halogen, hydroxyl, cyano, alkyl, haloalkyl, hydroxylalkyl, —C(O)N(R^{a})₂, —C(O)OR^{a}, —S(O)₂(R^{b}), —S(O)(NH)(R^{b}) and —P(O)(R^{b})₂;
Ring B is 5- to 6-membered heterocyclyl or 5- to 6-membered heteroaryl;
R², in each occurrence, is halogen, alkyl, haloalkyl, or cycloalkyl; R³ is

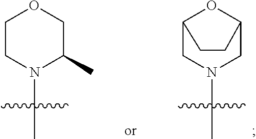

R^{a} and R^{d} are each independently hydrogen, halogen or alkyl;
R^{b} is alkyl, 3- to 6-membered cycloalkyl, 5- to 6-membered heterocyclyl, 5- to 6-membered aryl, or 5- to 6-membered heteroaryl, wherein said cycloalkyl, heterocyclyl, and heteroaryl are optionally substituted with one or more R^{c};
R^{c} is selected from the group consisting of hydroxyl, halogen, cyano, amino, alkyl, alkoxyl, and haloalkyl;
R^{e} is hydroxyl, halogen or alkyl;
n is 0, 1, 2, or 3; and
m is 0, 1, 2 or 3.

In one aspect, the present disclosure provides a compound having Formula (I):

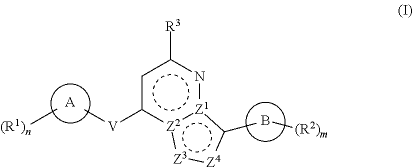

or a pharmaceutically acceptable salt thereof,
wherein
Z¹ is C or N;
Z² is C or N;
Z³ is CH, N, or S;
Z⁴ is CH or N;
V is a direct bond or —N(R^{a})—;
Ring A is absent, 3- to 6-membered cycloalkyl, 5- to 6-membered heterocyclyl, 5- to 6-membered aryl, or 5- to 6-membered heteroaryl;
R¹ is hydrogen, halogen, alkyl, —S(O)₂(R^{b}), or —S(O)(NH)(R^{b});
Ring B is 5- to 6-membered heterocyclyl or 5- to 6-membered heteroaryl;
R² is halogen, alkyl, haloalkyl, or cycloalkyl;
R³ is

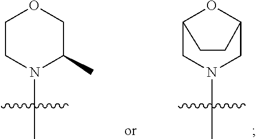

R^{a} is hydrogen or alkyl;
R^{b} is alkyl, 3- to 6-membered cycloalkyl, 5- to 6-membered heterocyclyl, 5- to 6-membered aryl, or 5- to 6-membered heteroaryl, wherein said cycloalkyl, heterocyclyl, and heteroaryl are optionally substituted with one or more R^{c};
R^{c} is selected from the group consisting of hydroxyl, halogen, cyano, amino, alkyl, alkoxyl, and haloalkyl;
n is 0, 1, 2, or 3; and
m is 0, 1, 2 or 3.

In some embodiments, Z¹ is C.

In some embodiments, Z¹ is N.

In some embodiments, Z² is C.

In some embodiments, Z² is N.

In some embodiments, Z¹ is C and Z² is N.

In some embodiments, Z¹ is N and Z² is C.

In some embodiments, Z¹ is C and Z² is C.

In some embodiments, Z¹ is CR^{a}. In certain embodiments, R^{a} is hydrogen.

In certain embodiments, R^{d} is alkyl. In certain embodiments, R^{d} is C₁₋₆ alkyl, C₁₋₅ alkyl, C₁₋₄ alkyl or C₁₋₃ alkyl. In certain embodiments, R^{d} is methyl.

In some embodiments, Z³ is CH.

In some embodiments, Z³ is N.

In some embodiments, Z³ is S.

In some embodiments, Z³ is O.

In some embodiments, Z³ is S(O).

In some embodiments, Z³ is S(O)₂.

In some embodiments, Z¹ is C, Z² is N and Z³ is CH or N.

In some embodiments, Z¹ is N, Z² is C and Z³ is CH, C(CH₃) or N.

In some embodiments, Z¹ is C, Z² is C and Z³ is O, S, S(O) or S(O)₂.

In some embodiments, Z⁴ is C.

In some embodiments, Z⁴ is N.

In some embodiments, V is a direct bond.

In some embodiments, V is alkyl optionally substituted with one or more R^{e}.

In certain embodiments, V is C₁₋₆ alkyl, C₁₋₅ alkyl, C₁₋₄ alkyl or C₁₋₃ alkyl.

In some embodiments, V is —N(R^{a}).

In certain embodiments, R^{a} is hydrogen.

In certain embodiments, R^{a} is alkyl. In some embodiments, R^{a} is C₁₋₆ alkyl, C₁₋₅ alkyl, C₁₋₄ alkyl or C₁₋₃ alkyl. In some embodiments, R^{a} is methyl, ethyl, n-propyl, or isopropyl.

In some embodiments, Ring A is absent.

In some embodiments, Ring A is 3- to 6-membered cycloalkyl.

In some embodiments, Ring A is cyclopropyl. In certain embodiments, Ring A is

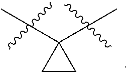 [SMILES: *C1(*)CC1]

In some embodiments, Ring A is 5- to 6-membered heterocyclyl.

In certain embodiments, Ring A is 5- to 6-membered heterocyclyl containing at least one nitrogen atom. In certain embodiments, Ring A is 5- to 6-membered heterocyclyl containing at least two nitrogen atoms. In certain embodiments, Ring A is 5- to 6-membered heterocyclyl containing two nitrogen atoms.

In some embodiments, Ring A is piperazinyl, tetrahydropyranyl or 1,2-thiazinane 1,1-dioxide.

In some embodiments, Ring A is 5- to 6-membered aryl.

In some embodiments, Ring A is phenyl.

In some embodiments, Ring A is 5- to 6-membered heteroaryl.

In certain embodiments, Ring A is 5- to 6-membered heteroaryl containing at least one nitrogen atom.

In certain embodiments, Ring A is 5-membered heteroaryl containing at least one nitrogen atom. In certain embodiments, Ring A is 5-membered heteroaryl containing at least two nitrogen atoms. In certain embodiments, Ring A is 5-membered heteroaryl containing at least three nitrogen atoms. In certain embodiments, Ring A is 5-membered heteroaryl containing at least one nitrogen atom and additional heteroatom(s) selected from O, N or S. In certain embodiments, Ring A is 5-membered heteroaryl containing two nitrogen atoms. In certain embodiments, Ring A is pyrazolyl. In certain embodiments, Ring A is 5-membered heteroaryl containing three nitrogen atoms. In certain embodiments, Ring A is triazolyl.

In certain embodiments, Ring A is 6-membered heteroaryl containing at least one nitrogen atom. In certain embodiments, Ring A is 6-membered heteroaryl containing at least one nitrogen atom and additional heteroatom(s) selected from O, N or S. In certain embodiments, Ring A is 6-membered heteroaryl containing one nitrogen atom. In certain embodiments, Ring A is pyridyl.

In some embodiments, Ring A is selected from the group consisting of:

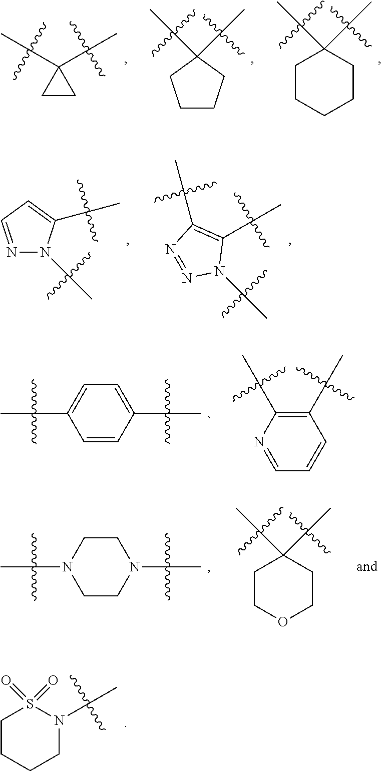

In some embodiments, R¹ is hydrogen.

In some embodiments, R¹ is cyano.

In some embodiments, R¹ is halogen. In certain embodiments, R¹ is fluoro.

In some embodiments, R¹ is alkyl. In certain embodiments, R¹ is C₁₋₆ alkyl, C₁₋₅ alkyl, C₁₋₄ alkyl or C₁₋₃ alkyl. In certain embodiments, R¹ is methyl.

In some embodiments, R¹ is haloalkyl. In certain embodiments, R¹ is C₁₋₆ haloalkyl, C₁₋₅ haloalkyl, C₁₋₄ haloalkyl or C₁₋₃ haloalkyl. In certain embodiments, R¹ is trifluoromethyl.

In some embodiments, R¹ is hydroxylalkyl. In certain embodiments, R¹ is C₁₋₆ hydroxylalkyl, C₁₋₅ hydroxylalkyl, C₁₋₄ hydroxylalkyl or C₁₋₃ hydroxylalkyl. In certain embodiments, R¹ is hydroxylmethyl.

In some embodiments, R¹ is —C(O)N(R^{a})₂ or —C(O)OR^{a}. In certain embodiments, R^{a} is hydrogen. In certain embodiments, R^{a} is alkyl. In certain embodiments, R^{a} is C₁₋₆ alkyl, C₁₋₅ alkyl, C₁₋₄ alkyl or C₁₋₃ alkyl. In certain embodiments, R^{a} is methyl.

In some embodiments, R¹ is —S(O)₂(R^{b}), —S(O)(NH)(R^{b}) or —P(O)(R^{b})₂.

In some embodiments, R^{b} is alkyl. In certain embodiments, R^{b} is C₁₋₆ alkyl, C₁₋₅ alkyl, C₁₋₄ alkyl or C₁₋₃ alkyl. In certain embodiments, R^{b} is methyl.

In some embodiments, n is 0, 1 or 2.

In some embodiments, Ring A is 3- to 6-membered cycloalkyl, and R¹ is cyano, hydroxyl, hydroxylalkyl, —C(O)N(R^{a})₂, —C(O)OR^{a}, —S(O)₂(R^{b}), or —S(O)(NH)(R^{b}).

In some embodiments, Ring A is 5- to 6-membered heterocyclyl, and R¹ is cyano, alkyl, —S(O)₂(R^{b}), or —S(O)(NH)(R.)

In some embodiments, Ring A is 5- to 6-membered aryl, and R¹ is cyano, —S(O)₂(R^{b}), or —S(O)(NH)(R^{b}).

In some embodiments, Ring A is 5- to 6-membered heteroaryl, and R¹ is cyano, halogen, hydroxyl, alkyl, or haloalkyl.

In some embodiments, Ring A is pyrazolyl, pyridyl or triazolyl, and R¹ is halogen, alkyl or haloalkyl.

In some embodiments, Ring A is cyclopropyl, cyclopentyl, cyclohexyl, piperazinyl or phenyl, R¹ is cyano, hydroxyl, hydroxylalkyl, —C(O)N(R^{a})₂, —C(O)OR^{a}, —S(O)₂(R^{b}), or —S(O)(NH)(R^{b}), and R^{b} is alkyl, for example C₁₋₆ alkyl, C₁₋₅ alkyl, C₁₋₄ alkyl or C₁₋₃ alkyl.

In some embodiments, Ring B is 5- to 6-membered heteroaryl.

In certain embodiments, Ring B is 5- to 6-membered heteroaryl containing at least one nitrogen atom.

In certain embodiments, Ring B is 5-membered heteroaryl containing at least one nitrogen atom. In certain embodiments, Ring B is 5-membered heteroaryl containing at least two nitrogen atoms. In certain embodiments, Ring B is 5-membered heteroaryl containing at least one nitrogen atom and additional heteroatom(s) selected from O, N or S. In certain embodiments, Ring B is 5-membered heteroaryl containing one nitrogen atom. In certain embodiments, Ring B is 5-membered heteroaryl containing two nitrogen atoms. In certain embodiments, Ring B is pyrazolyl or pyrrolyl. In certain embodiments, Ring B is 5-membered heteroaryl containing three nitrogen atoms. In certain embodiments, Ring B is triazolyl.

In certain embodiments, Ring B is 6-membered heteroaryl containing at least one nitrogen atom. In certain embodiments, Ring B is 6-membered heteroaryl containing at least two nitrogen atoms. In certain embodiments, Ring B is 6-membered heteroaryl containing at least one nitrogen atom and additional heteroatom(s) selected from O, N or S. In certain embodiments, Ring B is 6-membered heteroaryl containing one nitrogen atom. In certain embodiments, Ring B is 6-membered heteroaryl containing two nitrogen atoms. In certain embodiments, Ring B is pyridyl.

In some embodiments, R² is halogen. In certain embodiments, R² is chloro.

In some embodiments, R² is alkyl. In some embodiments, R² is C₁₋₆ alkyl, C₁₋₅ alkyl, C₁₋₄ alkyl or C₁₋₃ alkyl. In some embodiments, R² is methyl, ethyl, n-propyl, or isopropyl.

In some embodiments, R² is haloalkyl. In some embodiments, R² is C₁₋₃ haloalkyl. In certain embodiments, R² is trifluoromethyl.

In some embodiments, R² is cycloalkyl. In certain embodiments, R² is 3- to 6-membered cycloalkyl. In certain embodiments, R² is cyclopropyl.

In some embodiments, m is 0, 1 or 2.

In some embodiments,

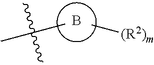

is selected from the group consisting of:

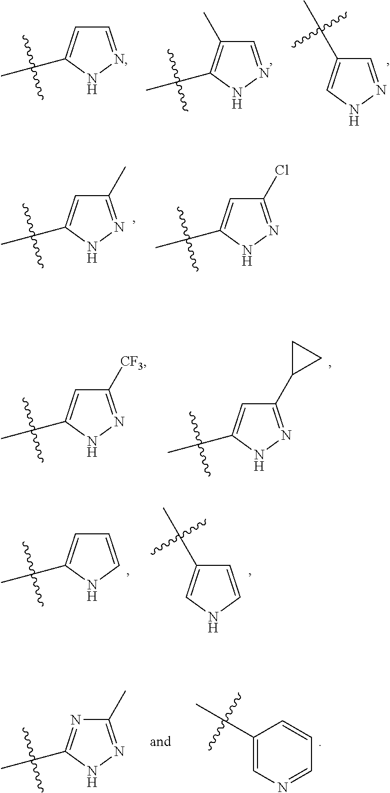

In some embodiments, R³ is

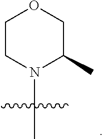 [SMILES: *N1CCOC[C@H]1C]

In some embodiments, R³ is

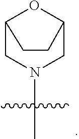 [SMILES: *N1CC2CCC(C1)O2]

In some embodiments, the present disclosure provides compound having a formula selected from the group consisting of:

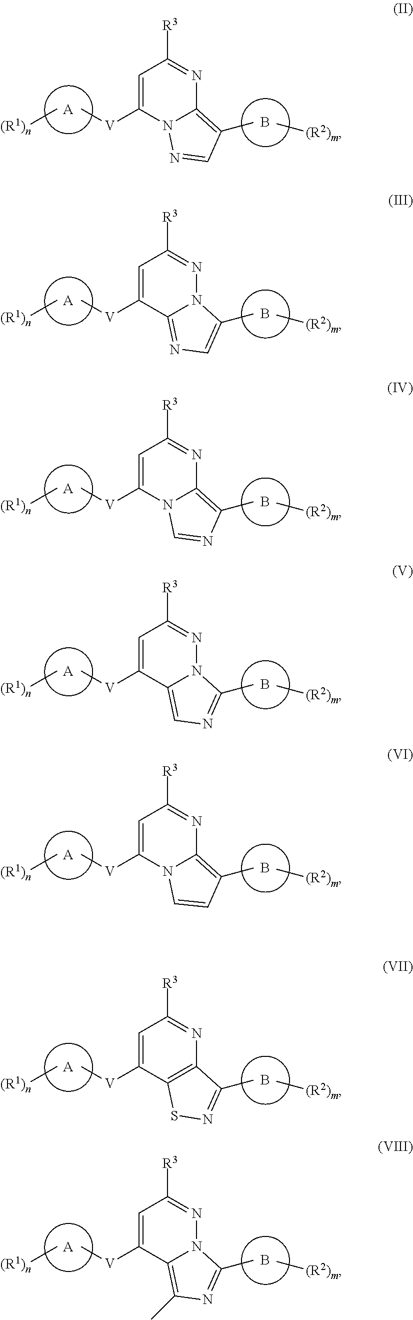

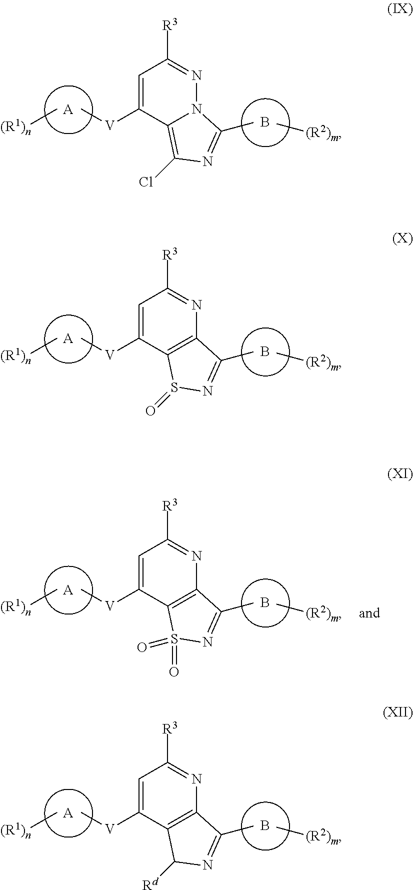

wherein V, Ring A, Ring B, R¹, R², R³, m and n are as defined supra.

In certain embodiments, in the compounds of Formula (II) to (XII),
V is a direct bond or C₁₋₃ alkyl;
Ring A is selected from cyclopropyl, cyclopentyl, cyclohexyl, piperazinyl, phenyl, pyrazolyl, pyridinyl, or triazolyl;
R¹ is selected from hydrogen, fluoro, cyano, methyl, —S(O)₂(R^{b}), —S(O)(NH)(R^{b}) or —P(O)(R^{b})₂;
Ring B is pyrazolyl, pyrrolyl, or pyridyl;
R² is chloro, C₁₋₃ alkyl, C₁₋₃ haloalkyl, or 3- to 6-membered cycloalkyl;
R³ is

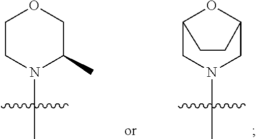

R^{b} is C₁₋₃ alkyl;
R^{d} is hydrogen, chloro or C₁₋₃ alkyl;
n is 0, 1 or 2; and
m is 0, 1 or 2.

In some embodiments, the present disclosure provides a compound having a formula selected from the group consisting of:

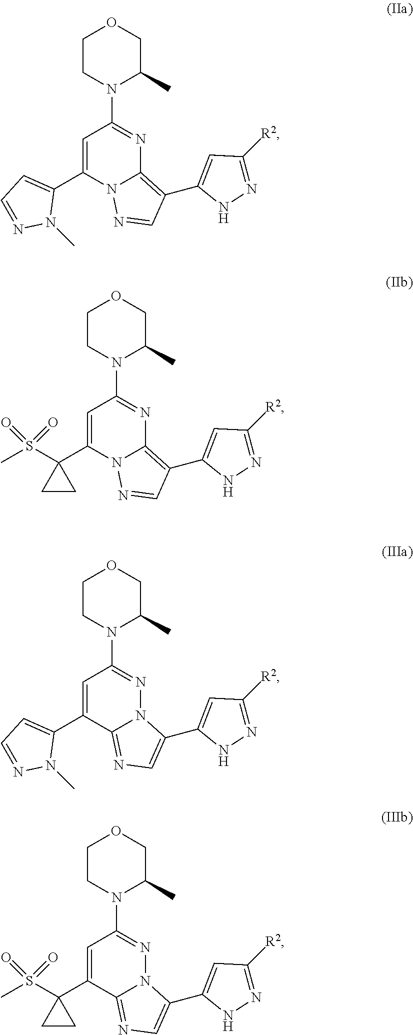

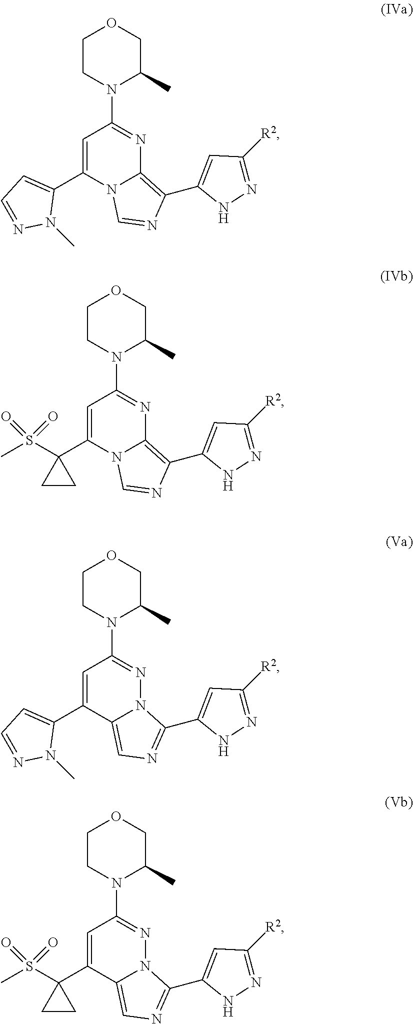

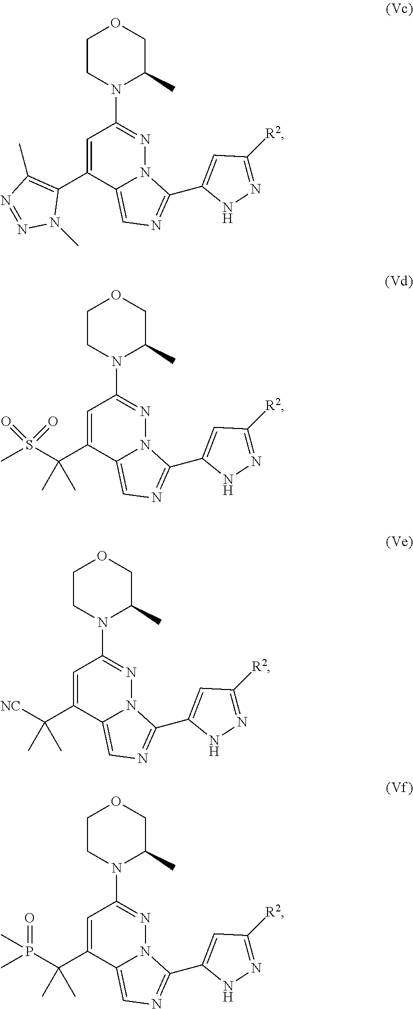

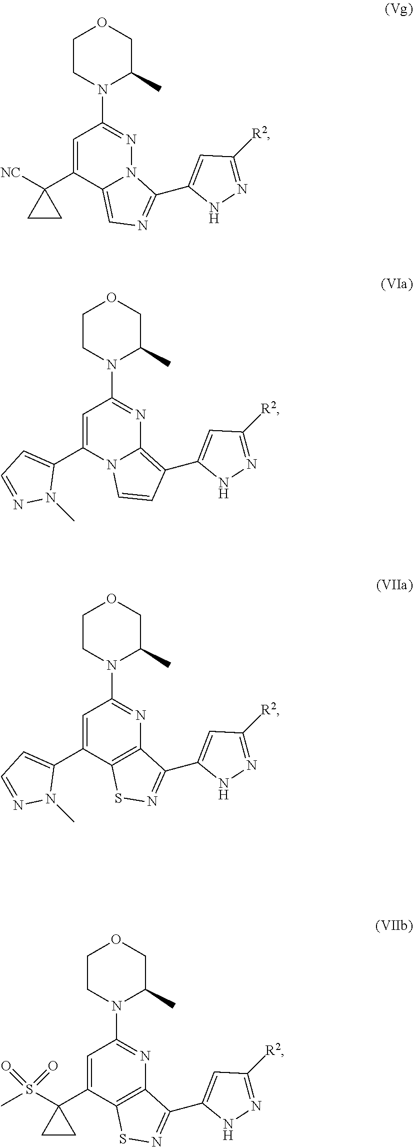

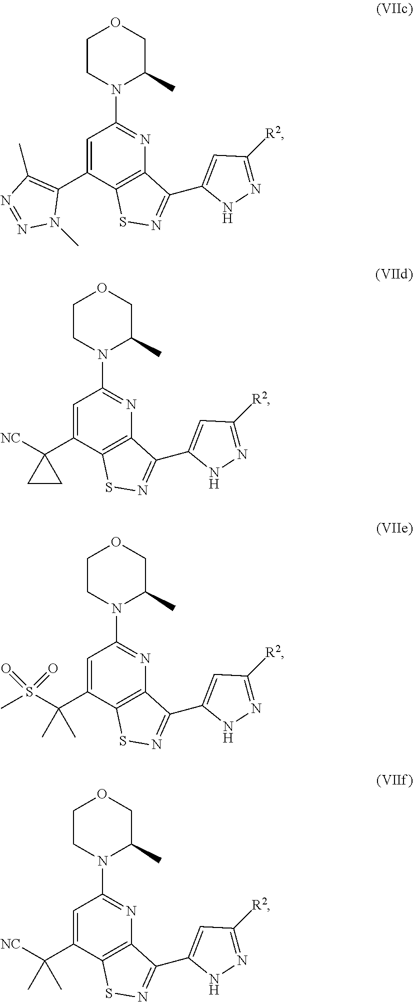

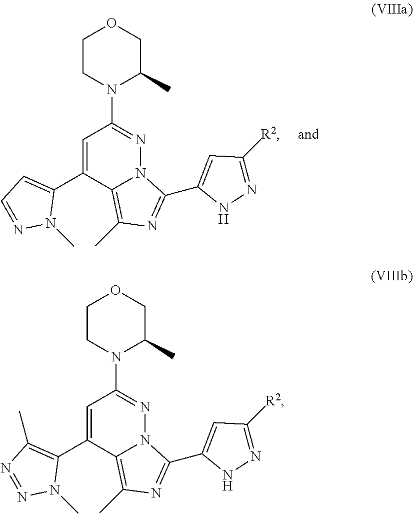

or a pharmaceutically acceptable salt thereof.

In some embodiments, the present disclosure provides a compound having a formula selected from the group consisting of:

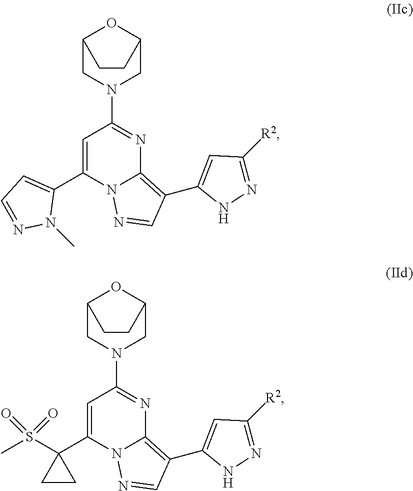

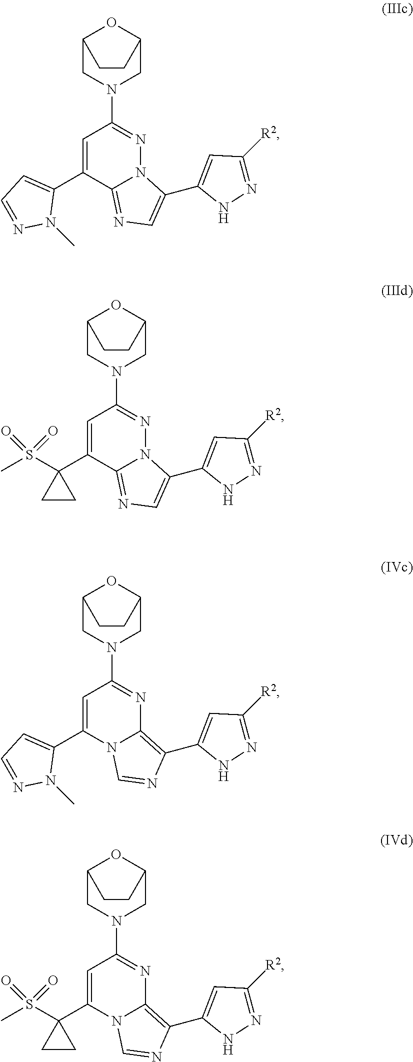

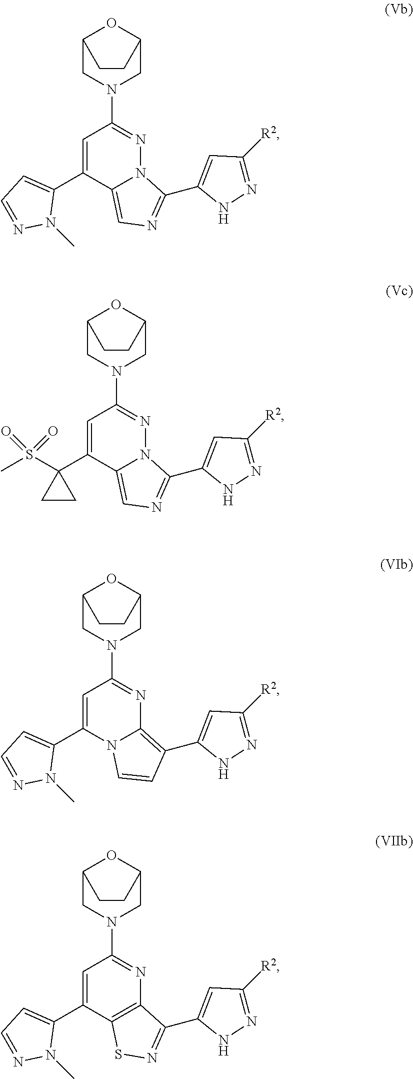

or a pharmaceutically acceptable salt thereof.

In some embodiments, the present disclosure provides a compound selected from the group consisting of:
(R)-3-methyl-4-(7-(1-methyl-1H-pyrazol-5-yl)-3-(1H-pyrazol-5-yl)pyrazolo[1,5-a]pyrimidin-5-yl)morpholine
(R)-3-methyl-4-(7-(1-methyl-1H-pyrazol-5-yl)-3-(1H-pyrazol-4-yl)pyrazolo[1,5-a]pyrimidin-5-yl)morpholine
(R)-3-methyl-4-(7-(1-methyl-1H-pyrazol-5-yl)-3-(pyridin-3-yl)pyrazolo[1,5-a]pyrimidin-5-yl)morpholine
(R)-3-methyl-4-(7-(1-methyl-1H-pyrazol-5-yl)-3-(1H-pyrrol-2-yl)pyrazolo[1,5-a]pyrimidin-5-yl)morpholine
(R)-3-methyl-4-(7-(1-(methylsulfonyl)cyclopropyl)-3-(1H-pyrazol-5-yl)pyrazolo[1,5-a]pyrimidin-5-yl)morpholine
(R)-4-(7-(2-fluoropyridin-3-yl)-3-(1H-pyrazol-5-yl)pyrazolo[1,5-a]pyrimidin-5-yl)-3-methylmorpholine
(R)-imino(methyl)(1-(5-((R)-3-methylmorpholino)-3-(1H-pyrazol-5-yl)pyrazolo[1,5-a]pyrimidin-7-yl)cyclopropyl)-λ6-sulfanone
(S)-imino(methyl)(1-(5-((R)-3-methylmorpholino)-3-(1H-pyrazol-5-yl)pyrazolo[1,5-a] pyrimidin-7-yl)cyclopropyl)-λ6-sulfanone
(R)-3-methyl-4-(7-(1-(methylsulfonyl)cyclopropyl)-3-(1H-pyrrol-2-yl)pyrazolo[1,5-a]pyrimidin-5-yl)morpholine
(R)-3-methyl-4-(7-(1-(methylsulfonyl)cyclopropyl)-3-(1H-pyrrol-3-yl)pyrazolo[1,5-a]pyrimidin-5-yl)morpholine
(R)-4-(3,7-di(1H-pyrazol-5-yl)pyrazolo[1,5-a]pyrimidin-5-yl)-3-methylmorpholine
(R)-3-methyl-4-(3-(3-methyl-1H-pyrazol-5-yl)-7-(1-(methylsulfonyl)cyclopropyl)pyrazolo[1,5-a]pyrimidin-5-yl)morpholine
(R)-3-methyl-4-(7-(1-(methylsulfonyl)cyclopropyl)-3-(3-(trifluoromethyl)-1H-pyrazol-5-yl)pyrazolo[1,5-a]pyrimidin-5-yl)morpholine
(R)-4-(3-(3-chloro-1H-pyrazol-5-yl)-7-(1-(methylsulfonyl)cyclopropyl)pyrazolo[1,5-a]pyrimidin-5-yl)-3-methylmorpholine
(R)-3-methyl-4-(3-(4-methyl-1H-pyrazol-5-yl)-7-(1-(methylsulfonyl)cyclopropyl)pyrazolo[1,5-a]pyrimidin-5-yl)morpholine
(3R)-3-methyl-4-[7-(1-methyl-1H-pyrazol-4-yl)-3-(1H-pyrazol-5-yl)pyrazolo[1,5-a]pyrimidin-5-yl]morpholine
(R)-3-methyl-4-(7-(4-(methylsulfonyl)phenyl)-3-(1H-pyrazol-5-yl)pyrazolo[1,5-a]pyrimidin-5-yl)morpholine
(R)-3-methyl-4-(7-(4-(methylsulfonyl)piperazin-1-yl)-3-(1H-pyrazol-5-yl)pyrazolo[1,5-a]pyrimidin-5-yl)morpholine
(R)-3-methyl-4-(7-(1-methyl-1H-pyrazol-5-yl)-3-(3-methyl-1H-pyrazol-5-yl)pyrazolo[1,5-a]pyrimidin-5-yl)morpholine
(R)-4-(3-(3-cyclopropyl-1H-pyrazol-5-yl)-7-(1-(methylsulfonyl)cyclopropyl)pyrazolo[1,5-a]pyrimidin-5-yl)-3-methylmorpholine
(R)—N-methyl-N-(5-(3-methylmorpholino)-3-(1H-pyrazol-5-yl)pyrazolo[1,5-a]pyrimidin-7-yl)methanesulfonamide
(R)-3-methyl-4-(8-(1-methyl-1H-pyrazol-5-yl)-3-(1H-pyrazol-5-yl)imidazo[1,2-b]pyridazin-6-yl)morpholine
(R)-3-methyl-4-(8-(1-(methylsulfonyl)cyclopropyl)-3-(1H-pyrazol-5-yl)imidazo[1,2-b]pyridazin-6-yl)morpholine
(R)-3-methyl-4-(8-(1-methyl-1H-pyrazol-5-yl)-3-(3-methyl-1H-pyrazol-5-yl)imidazo[1,2-b]pyridazin-6-yl)morpholine
(R)-3-methyl-4-(4-(1-(methylsulfonyl)cyclopropyl)-8-(1H-pyrazol-5-yl)imidazo[1,5-a]pyrimidin-2-yl)morpholine
(R)-3-methyl-4-(4-(1-methyl-1H-pyrazol-5-yl)-8-(1H-pyrazol-5-yl)imidazo[1,5-a]pyrimidin-2-yl)morpholine
(R)-4-(4-(1,4-dimethyl-1H-pyrazol-5-yl)-8-(3-methyl-1H-pyrazol-5-yl)imidazo[1,5-a]pyrimidin-2-yl)-3-methylmorpholine
(R)-3-methyl-4-(4-(1-methyl-1H-pyrazol-5-yl)-7-(1H-pyrazol-5-yl)imidazo[1,5-b]pyridazin-2-yl)morpholine
(R)-3-methyl-4-(4-(1-methyl-1H-pyrazol-5-yl)-8-(1H-pyrazol-5-yl)pyrrolo[1,2-a]pyrimidin-2-yl)morpholine
(R)-3-methyl-4-(7-(1-methyl-1H-pyrazol-5-yl)-3-(1H-pyrazol-5-yl)isothiazolo[4,5-b]pyridin-5-yl)morpholine
(R)-3-methyl-4-(4-(1-(methylsulfonyl)cyclopropyl)-7-(1H-pyrazol-5-yl) imidazo[1,5-b]pyridazin-2-yl)morpholine
(R)-3-methyl-4-(7-(3-methyl-1H-pyrazol-5-yl)-4-(1-(methyl sulfonyl)cyclopropyl)imidazo[1,5-b]pyridazin-2-yl)morpholine
(1R,5S)-3-(4-(1-(methylsulfonyl)cyclopropyl)-7-(1H-pyrazol-5-yl)imidazo[1,5-b]pyridazin-2-yl)-8-oxa-3-azabicyclo[3.2.1]octane
(3R)-4-[4-(dimethyl-1H-1,2,3-triazol-5-yl)-7-(1H-pyrazol-5-yl) imidazo[1,5-b]pyridazin-2-yl]-3-methylmorpholine
(R)-3-methyl-4-(4-(1-methyl-1H-pyrazol-5-yl)-7-(3-methyl-1H-pyrazol-5-yl)imidazo[1,5-b]pyridazin-2-yl)morpholine
(3R)-4-(4-(1,4-dimethyl-1H-1,2,3-triazol-5-yl)-7-(3-methyl-1H-pyrazol-5-yl)imidazo[1,5-b]pyridazin-2-yl)-3-methylmorpholine
(R)-3-methyl-4-(5-methyl-4-(1-methyl-1H-pyrazol-5-yl)-7-(1H-pyrazol-5-yl)imidazo[1,5-b]pyridazin-2-yl)morpholine
(R)-3-methyl-4-(7-(1-methyl-1H-pyrazol-5-yl)-3-(3-methyl-1H-pyrazol-5-yl)isothiazolo[4,5-b]pyridin-5-yl)morpholine
(R)-4-(7-(1,4-dimethyl-1H-1,2,3-triazol-5-yl)-3-(1H-pyrazol-5-yl)isothiazolo[4,5-b]pyridin-5-yl)-3-methylmorpholine
(3R)-4-[4-(diethylphosphoryl)-7-(1H-pyrazol-5-yl)imidazo [1,5-b]pyridazin-2-yl]-3-methylmorpholine
(R)-2-methyl-2-(2-(3-methylmorpholino)-7-(1H-pyrazol-5-yl)imidazo[1,5-b]pyridazin-4-yl)propanenitrile
(3R)-4-[4-(2-methanesulfonylpropan-2-yl)-7-(1H-pyrazol-5-yl)imidazo[1,5-b]pyridazin-2-yl]-3-methylmorpholine
(R)-3-methyl-4-(7-(3-methyl-1H-pyrazol-5-yl)-4-(2-(methyl sulfonyl)propan-2-yl)imidazo[1,5-b]pyridazin-2-yl)morpholine
(R)-dimethyl(2-(7-(3-methyl-1H-pyrazol-5-yl)-2-(3-methylmorpholino)imidazo[1,5-b]pyridazin-4-yl)propan-2-yl)phosphine oxide
(R)-1-(2-(3-methylmorpholino)-7-(1H-pyrazol-5-yl)imidazo[1,5-b]pyridazin-4-yl)cyclopropane-1-carbonitrile
(3R)-4-[4-(dimethyl-1H-1,2,3-triazol-5-yl)-5-methyl-7-(1H-pyrazol-5-yl)imidazo[1,5-b]pyridazin-2-yl]-3-methylmorpholine
(3R)-4-[4-(dimethyl-1H-1,2,3-triazol-5-yl)-5-methyl-7-(3-methyl-1H-pyrazol-5-yl)imidazo[1,5-b]pyridazin-2-yl]-3-methylmorpholine
(R)-3-methyl-4-(5-methyl-4-(1-methyl-1H-pyrazol-5-yl)-7-(3-methyl-1H-pyrazol-5-yl)imidazo[1,5-b]pyridazin-2-yl)morpholine
(R)-4-(7-(1,4-dimethyl-1H-1,2,3-triazol-5-yl)-3-(3-methyl-1H-pyrazol-5-yl)isothiazolo[4,5-b]pyridin-5-yl)-3-methylmorpholine
(R)-3-methyl-4-(7-(1-(methylsulfonyl)cyclopropyl)-3-(1H-pyrazol-5-yl)isothiazolo[4,5-b]pyridin-5-yl)morpholine
(R)-3-methyl-4-(3-(3-methyl-1H-pyrazol-5-yl)-7-(1-(methylsulfonyl)cyclopropyl)isothiazolo[4,5-b]pyridin-5-yl)morpholine
(R)-1-(5-(3-methylmorpholino)-3-(1H-pyrazol-5-yl)isothiazolo[4,5-b] pyridin-7-yl)cyclopropane-1-carbonitrile
(R)-1-(3-(3-methyl-1H-pyrazol-5-yl)-5-(3-methylmorpholino)isothiazolo [4,5-b]pyridin-7-yl)cyclopropane-1-carbonitrile
(R)-2-methyl-2-(5-(3-methylmorpholino)-3-(1H-pyrazol-5-yl) isothiazolo [4,5-b]pyridin-7-yl)propanenitrile
(R)-2-methyl-2-(3-(3-methyl-1H-pyrazol-5-yl)-5-(3-methylmorpholino)isothiazolo[4,5-b]pyridin-7-yl)propanenitrile
(R)-3-methyl-4-(7-(2-(methylsulfonyl)propan-2-yl)-3-(1H-pyrazol-5-yl)isothiazolo[4,5-b]pyridin-5-yl)morpholine
(R)-3-methyl-4-(3-(3-methyl-1H-pyrazol-5-yl)-7-(2-(methylsulfonyl)propan-2-yl)isothiazolo[4,5-b]pyridin-5-yl)morpholine
(R)-1-(5-(3-methylmorpholino)-3-(1H-pyrazol-5-yl)isothiazolo[4,5-b]pyridin-7-yl)cyclopentane-1-carbonitrile
(R)-1-(5-(3-methylmorpholino)-3-(1H-pyrazol-5-yl)isothiazolo[4,5-b]pyridin-7-yl)cyclohexane-1-carbonitrile
(R)-1-(2-(3-methylmorpholino)-7-(1H-pyrazol-5-yl)imidazo[1,5-b]pyridazin-4-yl)cyclopentane-1-carbonitrile
(R)-1-(2-(3-methylmorpholino)-7-(1H-pyrazol-5-yl)imidazo[1,5-b]pyridazin-4-yl)cyclohexane-1-carbonitrile
(3R)-4-[5-chloro-4-(1-methyl-1H-pyrazol-5-yl)-7-(1H-pyrazol-5-yl)imidazo[1,5-b]pyridazin-2-yl]-3-methylmorpholine
(R)-4-(5-chloro-4-(1-methyl-1H-pyrazol-5-yl)-7-(3-methyl-1H-pyrazol-5-yl)imidazo[1,5-b]pyridazin-2-yl)-3-methylmorpholine
(R)-1-(7-(3-methyl-1H-pyrazol-5-yl)-2-(3-methylmorpholino)imidazo[1,5-b]pyridazin-4-yl)cyclopropane-1-carbonitrile
(R)-2-methyl-2-(7-(3-methyl-1H-pyrazol-5-yl)-2-(3-methylmorpholino) imidazo[1,5-b]pyridazin-4-yl)propanenitrile
(R)-7-(1-methyl-1H-pyrazol-5-yl)-3-(3-methyl-1H-pyrazol-5-yl)-5-(3-methylmorpholino)isoxazolo[4,5-b]pyridine
(R)-7-(1-methyl-1H-pyrazol-5-yl)-5-(3-methylmorpholino)-3-(1H-pyrazol-5-yl)isoxazolo[4,5-b]pyridine
(R)-7-(1,4-dimethyl-1H-pyrazol-5-yl)-3-(3-methyl-1H-pyrazol-5-yl)-5-(3-methylmorpholino)isoxazolo[4,5-b]pyridine
(R)-7-(1,4-dimethyl-1H-pyrazol-5-yl)-5-(3-methylmorpholino)-3-(1H-pyrazol-5-yl)isoxazolo[4,5-b]pyridine
(R)-7-(1,4-dimethyl-1H-1,2,3-triazol-5-yl)-5-(3-methylmorpholino)-3-(1H-pyrazol-5-yl)isoxazolo[4,5-b]pyridine
(R)-7-(1,4-dimethyl-1H-1,2,3-triazol-5-yl)-3-(3-methyl-1H-pyrazol-5-yl)-5-(3-methylmorpholino)isoxazolo[4,5-b]pyridine
(R)-3-(3-methyl-1H-pyrazol-5-yl)-5-(3-methylmorpholino)-7-(1-(methylsulfonyl)cyclopropyl)isoxazolo[4,5-b]pyridine
(R)-1-(5-(3-methylmorpholino)-3-(1H-pyrazol-5-yl)isoxazolo[4,5-b]pyridin-7-yl)cyclopropane-1-carbonitrile
(R)-5-(3-methylmorpholino)-7-(2-(methylsulfonyl)propan-2-yl)-3-(1H-pyrazol-5-yl)isoxazolo[4,5-b]pyridine
(R)-3-(3-methyl-1H-pyrazol-5-yl)-5-(3-methylmorpholino)-7-(2-(methylsulfonyl)propan-2-yl)isoxazolo[4,5-b]pyridine
imino(methyl)(1-(3-(3-methyl-1H-pyrazol-5-yl)-5-((R)-3-methylmorpholino)isoxazolo[4,5-b]pyridin-7-yl)cyclopropyl)-λ6-sulfanone
imino(methyl)(2-(3-(3-methyl-1H-pyrazol-5-yl)-5-((R)-3-methylmorpholino)isoxazolo[4,5-b]pyridin-7-yl)propan-2-yl)-λ6-sulfanone
7-(1-methyl-1H-pyrazol-5-yl)-3-(3-methyl-1H-pyrazol-5-yl)-5-((R)-3-methylmorpholino)isothiazolo[4,5-b]pyridine 1-oxide
7-(1-methyl-1H-pyrazol-5-yl)-5-((R)-3-methylmorpholino)-3-(1H-pyrazol-5-yl)isothiazolo[4,5-b]pyridine 1-oxide
7-(1,4-dimethyl-1H-pyrazol-5-yl)-3-(3-methyl-1H-pyrazol-5-yl)-5-((R)-3-methylmorpholino)isothiazolo[4,5-b]pyridine 1-oxide
7-(1,4-dimethyl-1H-pyrazol-5-yl)-5-((R)-3-methylmorpholino)-3-(1H-pyrazol-5-yl)isothiazolo[4,5-b]pyridine 1-oxide
7-(1,4-dimethyl-1H-1,2,3-triazol-5-yl)-5-((R)-3-methylmorpholino)-3-(1H-pyrazol-5-yl)isothiazolo[4,5-b]pyridine 1-oxide
7-(1,4-dimethyl-1H-1,2,3-triazol-5-yl)-3-(3-methyl-1H-pyrazol-5-yl)-5-((R)-3-methylmorpholino)isothiazolo[4,5-b]pyridine 1-oxide
3-(3-methyl-1H-pyrazol-5-yl)-5-((R)-3-methylmorpholino)-7-(1-(methylsulfonyl)cyclopropyl)isothiazolo[4,5-b]pyridine 1-oxide
1-(5-((R)-3-methylmorpholino)-1-oxido-3-(1H-pyrazol-5-yl)isothiazolo[4,5-b]pyridin-7-yl)cyclopropane-1-carbonitrile
imino(methyl)(1-(3-(3-methyl-1H-pyrazol-5-yl)-5-((R)-3-methylmorpholino)-1-oxidoisothiazolo[4,5-b]pyridin-7-yl)cyclopropyl)-λ6-sulfanone
(R)-7-(1-methyl-1H-pyrazol-5-yl)-3-(3-methyl-1H-pyrazol-5-yl)-5-(3-methylmorpholino)isothiazolo[4,5-b]pyridine 1,1-dioxide
(R)-7-(1-methyl-1H-pyrazol-5-yl)-5-(3-methylmorpholino)-3-(1H-pyrazol-5-yl)isothiazolo[4,5-b]pyridine 1,1-dioxide
(R)-7-(1,4-dimethyl-1H-pyrazol-5-yl)-3-(3-methyl-1H-pyrazol-5-yl)-5-(3-methylmorpholino)isothiazolo[4,5-b]pyridine 1,1-dioxide
(R)-7-(1,4-dimethyl-1H-pyrazol-5-yl)-5-(3-methylmorpholino)-3-(1H-pyrazol-5-yl)isothiazolo[4,5-b]pyridine 1,1-dioxide
(R)-7-(1,4-dimethyl-1H-1,2,3-triazol-5-yl)-5-(3-methylmorpholino)-3-(1H-pyrazol-5-yl)isothiazolo[4,5-b]pyridine 1,1-dioxide
(R)-7-(1,4-dimethyl-1H-1,2,3-triazol-5-yl)-3-(3-methyl-1H-pyrazol-5-yl)-5-(3-methylmorpholino)isothiazolo[4,5-b]pyridine 1,1-dioxide
(R)-5-(3-methylmorpholino)-7-(1-(methylsulfonyl)cyclopropyl)-3-(1H-pyrazol-5-yl)isothiazolo[4,5-b]pyridine 1,1-dioxide
(R)-3-(3-methyl-1H-pyrazol-5-yl)-5-(3-methylmorpholino)-7-(2-(methylsulfonyl)propan-2-yl)isothiazolo[4,5-b]pyridine 1,1-dioxide
imino(methyl)(2-(3-(3-methyl-1H-pyrazol-5-yl)-5-((R)-3-methylmorpholino)-1,1-dioxidoisothiazolo[4,5-b]pyridin-7-yl)propan-2-yl)-λ6-sulfanone
4-(5-methyl-4-(1-methyl-1H-pyrazol-5-yl)-7-(1H-pyrazol-5-yl)imidazo[1,5-b]pyridazin-2-yl)morpholine
4-(5-methyl-4-(1-methyl-1H-pyrazol-5-yl)-7-(3-methyl-1H-pyrazol-5-yl)imidazo[1,5-b]pyridazin-2-yl)morpholine,
(R)-2-(3-(3-methyl-1H-pyrazol-5-yl)-5-(3-methylmorpholino)isothiazolo[4,5-b]pyridin-7-yl)propan-2-ol,
(R)-3-methyl-4-(7-(1-methyl-1H-1,2,3-triazol-5-yl)-3-(3-methyl-1H-pyrazol-5-yl)isothiazolo[4,5-b]pyridin-5-yl)morpholine,
(R)-3-methyl-4-(7-(1-methyl-1H-1,2,3-triazol-5-yl)-3-(1H-pyrazol-5-yl)isothiazolo[4,5-b]pyridin-5-yl)morpholine,
(R)-4-(7-(1,4-dimethyl-1H-pyrazol-5-yl)-3-(1H-pyrazol-5-yl)isothiazolo[4,5-b]pyridin-5-yl)-3-methylmorpholine
(R)-4-(7-(1,4-dimethyl-1H-pyrazol-5-yl)-3-(3-methyl-1H-pyrazol-5-yl)isothiazolo[4,5-b]pyridin-5-yl)-3-methylmorpholine
(R)-4-(7-(3,5-dimethylisoxazol-4-yl)-3-(1H-pyrazol-5-yl)isothiazolo[4,5-b]pyridin-5-yl)-3-methylmorpholine
(R)-4-(7-(3,5-dimethylisoxazol-4-yl)-3-(3-methyl-1H-pyrazol-5-yl)isothiazolo[4,5-b]pyridin-5-yl)-3-methylmorpholine
(R)-2-(3-(3-methyl-1H-pyrazol-5-yl)-5-(3-methylmorpholino)isothiazolo[4,5-b]pyridin-7-yl)propan-2-ol
(R)-4-(7-(cyclopropylsulfonyl)-3-(1H-pyrazol-5-yl)isothiazolo[4,5-b]pyridin-5-yl)-3-methylmorpholine
(R)-2-(5-(3-methylmorpholino)-3-(1H-pyrazol-5-yl)isothiazolo[4,5-b]pyridin-7-yl)-1,2-thiazinane 1,1-dioxide
(R)—N-(3-chloro-1H-pyrazol-5-yl)-4-(3-methylmorpholino)-6-(1-(methylsulfonyl)cyclopropyl)pyrimidin-2-amine
(1R,5S)-3-(4-(1-methyl-1H-pyrazol-5-yl)-7-(1H-pyrazol-5-yl)imidazo[1,5-b]pyridazin-2-yl)-8-oxa-3-azabicyclo[3.2.1]octane
(1R,5S)-3-(4-(1-methyl-1H-pyrazol-5-yl)-7-(3-methyl-1H-pyrazol-5-yl)imidazo[1,5-b]pyridazin-2-yl)-8-oxa-3-azabicyclo[3.2.1]octane
(R)-1-(3-(3-methyl-1H-pyrazol-5-yl)-5-(3-methylmorpholino)isothiazolo[4,5-b]pyridin-7-yl)cyclopentane-1-carbonitrile
(R)-1-(3-(3-methyl-1H-pyrazol-5-yl)-5-(3-methylmorpholino)isothiazolo[4,5-b]pyridin-7-yl)cyclohexane-1-carbonitrile
(R)-4-(5-(3-methylmorpholino)-3-(1H-pyrazol-5-yl)isothiazolo[4,5-b]pyridin-7-yl)tetrahydro-2H-pyran-4-carbonitrile
(R)-4-(3-(3-methyl-1H-pyrazol-5-yl)-5-(3-methylmorpholino)isothiazolo[4,5-b]pyridin-7-yl)tetrahydro-2H-pyran-4-carbonitrile
(R)-4-(7-(cyclopropylsulfonyl)-3-(3-methyl-1H-pyrazol-5-yl)isothiazolo[4,5-b]pyridin-5-yl)-3-methylmorpholine
(R)-1-(3-(3-methyl-1H-pyrazol-5-yl)-5-(3-methylmorpholino)isothiazolo[4,5-b]pyridin-7-yl)cyclohexan-1-ol
(R)-1-(3-(3-methyl-1H-pyrazol-5-yl)-5-(3-methylmorpholino)isothiazolo[4,5-b]pyridin-7-yl)cyclopentane-1-carboxamide
(R)-1-(3-(3-methyl-1H-pyrazol-5-yl)-5-(3-methylmorpholino)isothiazolo[4,5-b]pyridin-7-yl)cyclohexane-1-carboxamide
(R)-1-(5-(3-methylmorpholino)-3-(1H-pyrazol-5-yl)isothiazolo[4,5-b]pyridin-7-yl)cyclohexane-1-carboxamide
(R)-1-(5-(3-methylmorpholino)-3-(1H-pyrazol-5-yl)isothiazolo[4,5-b]pyridin-7-yl)cyclopentane-1-carboxamide
(R)-1-(5-(3-methylmorpholino)-3-(1H-pyrazol-5-yl)isothiazolo[4,5-b]pyridin-7-yl)cyclohexan-1-ol
methyl (R)-1-(5-(3-methylmorpholino)-3-(1H-pyrazol-5-yl)isothiazolo[4,5-b]pyridin-7-yl)cyclopentane-1-carboxylate
(R)-3-methyl-4-(3-(3-methyl-1H-1,2,4-triazol-5-yl)-7-(1-methyl-1H-pyrazol-5-yl)isothiazolo[4,5-b]pyridin-5-yl)morpholine imino(methyl)(1-(3-(3-methyl-1H-pyrazol-5-yl)-5-((R)-3-methylmorpholino)isothiazolo[4,5-b]pyridin-7-yl)cyclopropyl)-λ6-sulfanone
(R)-3-methyl-4-(3-(3-methyl-1H-pyrazol-5-yl)-7-(2-(methylsulfonyl)phenyl)isothiazolo[4,5-b]pyridin-5-yl)morpholine
(R)-3-methyl-4-(3-(3-methyl-1H-pyrazol-5-yl)-7-(2-(trifluoromethyl)pyridin-3-yl)isothiazolo[4,5-b]pyridin-5-yl)morpholine
(R)-2-methyl-2-(3-(3-methyl-1H-pyrazol-5-yl)-5-(3-methylmorpholino)isothiazolo[4,5-b]pyridin-7-yl)propan-1-ol
(R)-(1-(3-(3-methyl-1H-pyrazol-5-yl)-5-(3-methylmorpholino)isothiazolo[4,5-b]pyridin-7-yl)cyclopropyl)methanol
(R)-3-methyl-4-(7-(1-methyl-1H-1,2,3-triazol-5-yl)-3-(3-methyl-1H-pyrazol-5-yl)isothiazolo[4,5-b]pyridin-5-yl)morpholine
(R)-2-methyl-2-(5-(3-methylmorpholino)-3-(1H-pyrazol-5-yl)isothiazolo[4,5-b]pyridin-7-yl)propan-1-ol
(R)-4-(3-(1H-pyrazol-5-yl)-7-(2-(trifluoromethyl)pyridin-3-yl)isothiazolo[4,5-b]pyridin-5-yl)-3-methylmorpholine
(R)-3-methyl-4-(7-(1-methyl-1H-1,2,4-triazol-5-yl)-3-(3-methyl-1H-pyrazol-5-yl)isothiazolo[4,5-b]pyridin-5-yl)morpholine
(R)-3-methyl-4-(7-(1-methyl-1H-1,2,3-triazol-5-yl)-3-(1H-pyrazol-5-yl)isothiazolo[4,5-b]pyridin-5-yl)morpholine
(R)-4-(7-chloro-3-(3-methyl-1H-pyrazol-5-yl)isothiazolo[4,5-b]pyridin-5-yl)-3-methylmorpholine
(R)-(4-(3-(3-methyl-1H-pyrazol-5-yl)-5-(3-methylmorpholino)isothiazolo[4,5-b]pyridin-7-yl)tetrahydro-2H-pyran-4-yl)methanol
(R)-1-(3-(3-methyl-1H-pyrazol-5-yl)-5-(3-methylmorpholino)isothiazolo[4,5-b]pyridin-7-yl)cyclopentan-1-ol
(R)-4-(7-(1-ethyl-1H-1,2,3-triazol-5-yl)-3-(3-methyl-1H-pyrazol-5-yl)isothiazolo[4,5-b]pyridin-5-yl)-3-methylmorpholine
(R)-dimethyl(3-(3-methyl-1-H-pyrazol-5-yl)-5-(3-methylmorpholino)isothiazolo[4,5-b]pyridin-7-yl)phosphine oxide
(R)-4-(5-fluoro-4-(i-methyl-1H-pyrazol-5-yl)-7-(1H-pyrazol-5-yl)imidazo[1,5-b]pyridazin-2-yl)-3-methylmorpholine,
   or a pharmaceutically acceptable salt thereof.

Exemplary compounds of the present disclosure are set forth in Table 1 below.

TABLE 1

| Compd. | |
|---|---|
| No. | Compound Structure and Name |
| 1 | 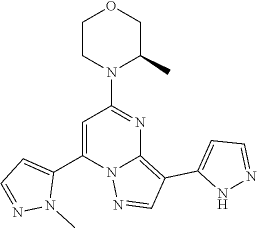 [SMILES: C[C@@H]1COCCN1c1cc(-c2ccnn2C)n2ncc(-c3ccn[nH]3)c2n1] |
| | (R)-3-methyl-4-(7-(1-methyl-1H-pyrazol-5-yl)-3-(1H-pyrazol-5- |
| | yl)pyrazolo[1,5-a]pyrimidin-5-yl)morpholine |
| 2 | 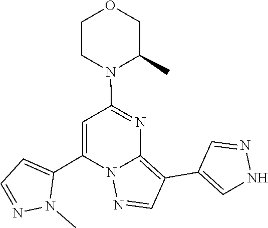 [SMILES: C[C@@H]1COCCN1c1cc(-c2ccnn2C)n2ncc(-c3cn[nH]c3)c2n1] |
| | (R)-3-methyl-4-(7-(1-methyl-1H-pyrazol-5-yl)-3-(1H-pyrazol-4- |
| | yl)pyrazolo[1,5-a]pyrimidin-5-yl)morpholine |
| 3 | 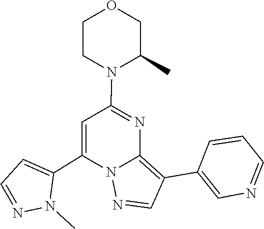 [SMILES: C[C@@H]1COCCN1c1cc(-c2ccnn2C)n2ncc(-c3cccnc3)c2n1] |
| | (R)-3-methyl-4-(7-(1-methyl-1H-pyrazol-5-yl)-3-(pyridin-3- |
| | yl)pyrazolo[1,5-a]pyrimidin-5-yl)morpholine |
| 4 | 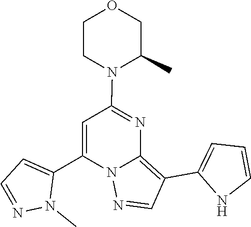 [SMILES: C[C@@H]1COCCN1c1cc(-c2ccnn2C)n2ncc(-c3ccc[nH]3)c2n1] |
| | (R)-3-methyl-4-(7-(1-methyl-1H-pyrazol-5-yl)-3-(1H-pyrrol-2- |
| | yl)pyrazolo[1,5-a]pyrimidin-5-yl)morpholine |
| 5 | 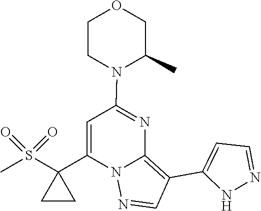 [SMILES: C[C@@H]1COCCN1c1cc(C2(S(C)(=O)=O)CC2)n2ncc(-c3ccn[nH]3)c2n1] |
| | (R)-3-methyl-4-(7-(1-(methylsulfonyl)cyclopropyl)-3-(1H-pyrazol- |
| | 5-yl)pyrazolo[1,5-a]pyrimidin-5-yl)morpholine |
| 6 | 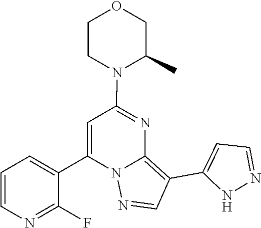 [SMILES: C[C@@H]1COCCN1c1cc(-c2cccnc2F)n2ncc(-c3ccn[nH]3)c2n1] |
| | (R)-4-(7-(2-fluoropyridin-3-yl)-3-(1H-pyrazol-5-yl)pyrazolo[1,5- |
| | alpyrimidin-5-yl)-3-methylmorpholine |
| 7a | 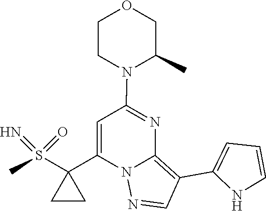 [SMILES: C[C@@H]1COCCN1c1cc(C2([S@](C)(=N)=O)CC2)n2ncc(-c3ccc[nH]3)c2n1] |
| | (R)-imino(methyl)(1-(5-((R)-3-methylmorpholino)-3-(1H-pyrazol- |
| | 5-yl)pyrazolo[1,5-a]pyrimidin-7-yl)cyclopropyl)-λ6-sulfanone |
| 7b | 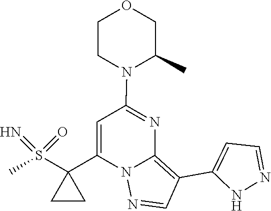 [SMILES: C[C@@H]1COCCN1c1cc(C2([S@@](C)(=N)=O)CC2)n2ncc(-c3ccn[nH]3)c2n1] |
| | (S)-imino(methyl)(1-(5-((R)-3-methylmorpholino)-3-(1H-pyrazol- |
| | 5-yl)pyrazolo[1,5-a] pyrimidin-7-yl)cyclopropyl)-λ6-sulfanone |
| 8 | 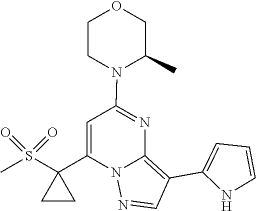 [SMILES: C[C@@H]1COCCN1c1cc(C2(S(C)(=O)=O)CC2)n2ncc(-c3ccc[nH]3)c2n1] |
| | (R)-3-methyl-4-(7-(1-(methylsulfonyl)cyclopropyl)-3-(1H- |
| | pyrrol-2-yl)pyrazolo[1,5-a ]pyrimidin-5-yl)morpholine |
| 9 | 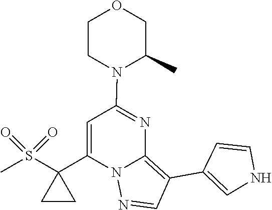 [SMILES: C[C@@H]1COCCN1c1cc(C2(S(C)(=O)=O)CC2)n2ncc(-c3cc[nH]c3)c2n1] |
| | (R)-3-methyl-4-(7-(1-(methylsulfonyl)cyclopropyl)-3-(1H- |
| | pyrrol-3-yl)pyrazolo[1,5-a]pyrimidin-5-yl)morpholine |
| 10 | 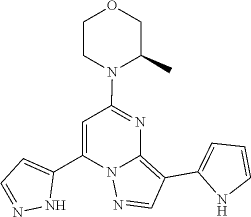 [SMILES: C[C@@H]1COCCN1c1cc(-c2ccn[nH]2)n2ncc(-c3ccc[nH]3)c2n1] |
| | (R)-4-(3,7-di(1H-pyrazol-5-yl)pyrazolo[1,5-a]pyrimidin-5-yl)-3- |
| | methylmorpholine |
| 11 | 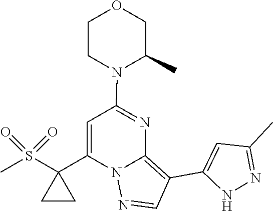 |
| | (R)-3-methyl-4-(3-(3-methyl-1H-pyrazol-5-yl)-7-(1- |
| | (methylsulfonyl)cyclopropyl)pyrazolo[1,5-a]pyrimidin-5- |
| | yl)morpholine |
| 12 | 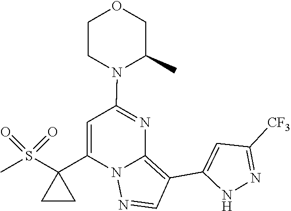 [SMILES: C[C@@H]1COCCN1c1cc(C2(S(C)(=O)=O)CC2)n2ncc(-c3cc(C(F)(F)F)n[nH]3)c2n1] |
| | (R)-3-methyl-4-(7-(1-(methylsulfonyl)cyclopropyl)-3-(3- |
| | (trifluoromethyl)-1H-pyrazol-5-yl)pyrazolo[1,5-a]pyrimidin-5- |
| | yl)morpholine |
| 13 | 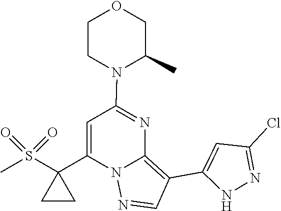 [SMILES: C[C@@H]1COCCN1c1cc(C2(S(C)(=O)=O)CC2)n2ncc(-c3cc(Cl)n[nH]3)c2n1] |
| | (R)-4-(3-(3-chloro-1H-pyrazol-5-yl)-7-(1- |
| | (methylsulfonyl)cyclopropyl)pyrazolo[1,5-a]pyrimidin-5-yl)-3- |
| | methylmorpholine |
| 14 | 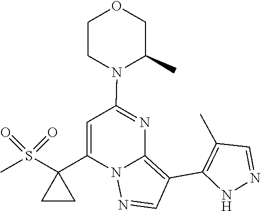 [SMILES: Cc1cn[nH]c1-c1cnn2c(C3(S(C)(=O)=O)CC3)cc(N3CCOC[C@H]3C)nc12] |
| | (R)-3-methyl-4-(3-(4-methyl-1H-pyrazol-5-yl)-7-(1- |
| | (methylsulfonyl)cyclopropyl)pyrazolo[1,5-a]pyrimidin-5- |
| | yl)morpholine |
| 15 | 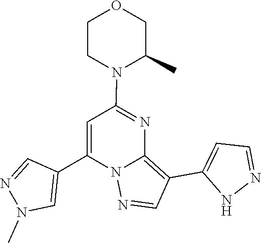 [SMILES: C[C@@H]1COCCN1c1cc(-c2cnn(C)c2)n2ncc(-c3ccn[nH]3)c2n1] |
| | (3R)-3-methyl-4-[7-(1-methyl-1H-pyrazol-4-yl)-3-(1H-pyrazol-5- |
| | yl)pyrazolo[1,5-a]pyrimidin-5-yl]morpholine |
| 16 | 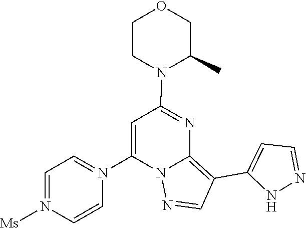 |
| | (R)-3-methyl-4-(7-(4-(methylsulfonyl)phenyl)-3-(1H-pyrazol-5- |
| | yl)pyrazolo[1,5-a]pyrimidin-5-yl)morpholine |
| 17 | 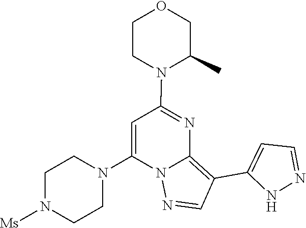 [SMILES: C[C@@H]1COCCN1c1cc(N2CCN(S(C)(=O)=O)CC2)n2ncc(-c3ccn[nH]3)c2n1] |
| | (R)-3-methyl-4-(7-(4-(methylsulfonyl)piperazin-1-yl)-3-(1H- |
| | pyrazol-5-yl)pyrazolo[1,5-a]pyrimidin-5-yl)morpholine |
| 18 | 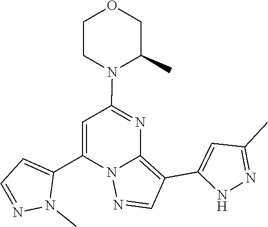 [SMILES: Cc1cc(-c2cnn3c(-c4ccnn4C)cc(N4CCOC[C@H]4C)nc23)[nH]n1] |
| | (R)-3-methyl-4-(7-(1-methyl-1H-pyrazol-5-yl)-3-(3-methyl-1H- |
| | pyrazol-5-yl)pyrazolo[1,5-a]pyrimidin-5-yl)morpholine |
| 19 | 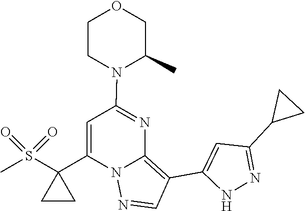 [SMILES: C[C@@H]1COCCN1c1cc(C2(S(C)(=O)=O)CC2)n2ncc(-c3cc(C4CC4)n[nH]3)c2n1] |
| | (R)-4-(3-(3-cyclopropyl-1H-pyrazol-5-yl)-7-(1- |
| | (methylsulfonyl)cyclopropyl)pyrazolo[1,5-a]pyrimidin-5-yl)-3- |
| | methylmorpholine |
| 20 | 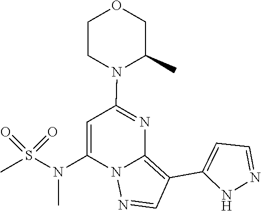 [SMILES: C[C@@H]1COCCN1c1cc(N(C)S(C)(=O)=O)n2ncc(-c3ccn[nH]3)c2n1] |
| | (R)-N-methyl-N-(5-(3-methylmorpholino)-3-(1H-pyrazol-5- |
| | yl)pyrazolo[1,5-a]pyrimidin-7-yl)methanesulfonamide |
| 21 | 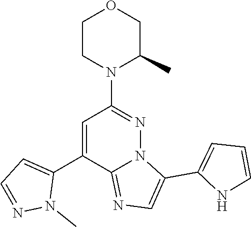 [SMILES: C[C@@H]1COCCN1c1cc(-c2ccnn2C)c2ncc(-c3ccc[nH]3)n2n1] |
| | (R)-3-methyl-4-(8-(1-methyl-1H-pyrazol-5-yl)-3-(1H-pyrazol-5- |
| | yl)imidazo[1,2-b]pyridazin-6-yl)morpholine |
| 22 | 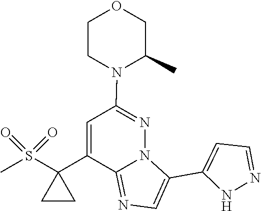 [SMILES: C[C@@H]1COCCN1c1cc(C2(S(C)(=O)=O)CC2)c2ncc(-c3ccn[nH]3)n2n1] |
| | (R)-3-methyl-4-(8-(1-(methylsulfonyl)cyclopropyl)-3-(1H- |
| | pyrazol-5-yl)imidazo[1,2-b]pyridazin-6-yl)morpholine |
| 23 | 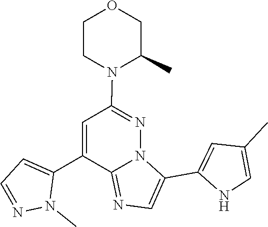 [SMILES: Cc1c[nH]c(-c2cnc3c(-c4ccnn4C)cc(N4CCOC[C@H]4C)nn23)c1] |
| | (R)-3-methyl-4-(8-(1-methyl-1H-pyrazol-5-yl)-3-(3-methyl-1H- |
| | pyrazol-5-yl)imidazo[1,2-b]pyridazin-6-yl)morpholine |
| 24 | 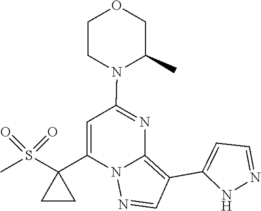 [SMILES: C[C@@H]1COCCN1c1cc(C2(S(C)(=O)=O)CC2)n2ncc(-c3ccn[nH]3)c2n1] |
| | (R)-3-methyl-4-(4-(1-(methylsulfonyl)cyclopropyl)-8-(1H- |
| | pyrazol-5-yl)imidazo[1,5-a]pyrimidin-2-yl)morpholine |
| 25 | 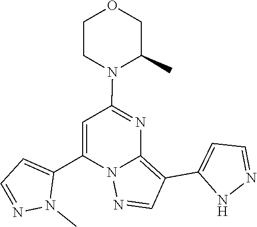 [SMILES: C[C@@H]1COCCN1c1cc(-c2ccnn2C)n2ncc(-c3ccn[nH]3)c2n1] |
| | (R)-3-methyl-4-(4-(1-methyl-1H-pyrazol-5-yl)-8-(1H-pyrazol-5- |
| | yl)imidazo[1,5-a]pyrimidin-2-yl)morpholine |
| 26 | 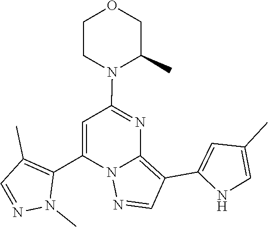 [SMILES: Cc1c[nH]c(-c2cnn3c(-c4c(C)cnn4C)cc(N4CCOC[C@H]4C)nc23)c1] |
| | (R)-4-(4-(1,4-dimethyl-1H-pyrazol-5-yl)-8-(3-methyl-1H- |
| | pyrazol-5-yl)imidazo[1,5-a]pyrimidin-2-yl)-3-methylmorpholine |
| 27 | 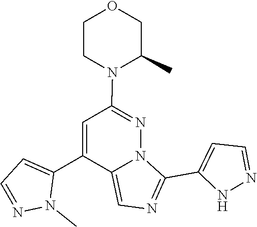 [SMILES: C[C@@H]1COCCN1c1cc(-c2ccnn2C)c2cnc(-c3ccn[nH]3)n2n1] |
| | (R)-3-methyl-4-(4-(1-methyl-1H-pyrazol-5-yl)-7-(1H-pyrazol-5- |
| | yl)imidazo[1,5-b]pyridazin-2-yl)morpholine |
| 28 | 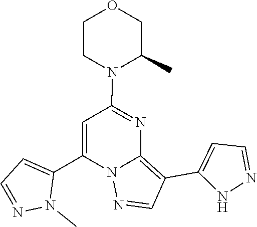 [SMILES: C[C@@H]1COCCN1c1cc(-c2ccnn2C)n2ncc(-c3ccn[nH]3)c2n1] |
| | (R)-3-methyl-4-(4-(1-methyl-1H-pyrazol-5-yl)-8-(1H-pyrazol-5- |
| | yl)pyrrolo[1,2-a]pyrimidin-2-yl)morpholine |
| 29 | 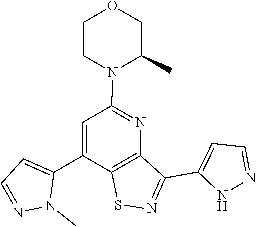 [SMILES: C[C@@H]1COCCN1c1cc(-c2ccnn2C)c2snc(-c3ccn[nH]3)c2n1] |
| | (R)-3-methyl-4-(7-(1-methyl-1H-pyrazol-5-yl)-3-(1H-pyrazol-5- |
| | yl)isothiazolo[4,5-b]pyridin-5-yl)morpholine |
| 30 | 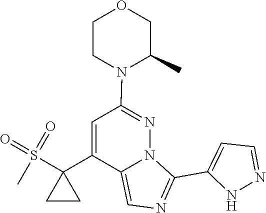 [SMILES: C[C@@H]1COCCN1c1cc(C2(S(C)(=O)=O)CC2)c2cnc(-c3ccn[nH]3)n2n1] |
| | (R)-3-methyl-4-(4-(1-(methylsulfonyl)cyclopropyl)-7-(1H- |
| | pyrazol-5-yl) imidazo[1,5-b]pyridazin-2-yl)morpholine |
| 31 | 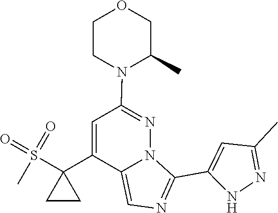 [SMILES: Cc1cc(-c2ncc3c(C4(S(C)(=O)=O)CC4)cc(N4CCOC[C@H]4C)nn23)[nH]n1] |
| | (R)-3-methyl-4-(7-(3-methyl-1H-pyrazol-5-yl)-4-(1-(methyl |
| | sulfonyl)cyclopropyl)imidazo[1,5-b]pyridazin-2-yl)morpholine |
| 32 | 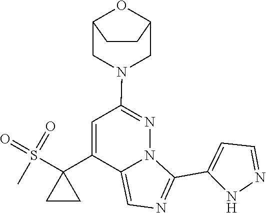 [SMILES: CS(=O)(=O)C1(c2cc(N3CC4CCC(C3)O4)nn3c(-c4ccn[nH]4)ncc23)CC1] |
| | (1R,5S)-3-(4-(1-(methylsulfonyl)cyclopropyl)-7-(1H-pyrazol-5- |
| | yl)imidazo[1,5-b]pyridazin-2-yl)-8-oxa-3-azabicyclo[3.2.1]octane |
| 33 | 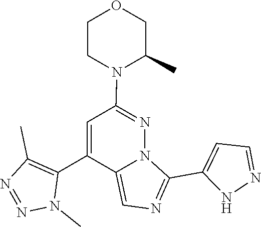 [SMILES: Cc1nnn(C)c1-c1cc(N2CCOC[C@H]2C)nn2c(-c3ccn[nH]3)ncc12] |
| | (3R)-4-[4-(dimethyl-1H-1,2,3-triazol-5-yl)-7-(1H-pyrazol-5-yl) |
| | imidazo[1,5-b]pyridazin-2-yl]-3-methylmorpholine |
| 34 | 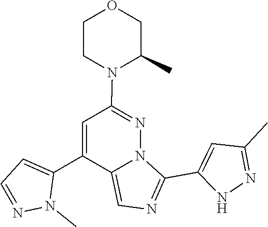 [SMILES: Cc1cc(-c2ncc3c(-c4ccnn4C)cc(N4CCOC[C@H]4C)nn23)[nH]n1] |
| | (R)-3-methyl-4-(4-(1-methyl-1H-pyrazol-5-yl)-7-(3-methyl-1H- |
| | pyrazol-5-yl)imidazo[1,5-b]pyridazin-2-yl)morpholine |
| 35 | 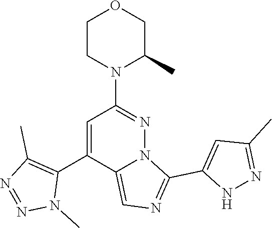 [SMILES: Cc1cc(-c2ncc3c(-c4c(C)nnn4C)cc(N4CCOC[C@H]4C)nn23)[nH]n1] |
| | (3R)-4-(4-(1,4-dimethyl-1H-1,2,3-triazol-5-yl)-7-(3-methyl -1H- |
| | pyrazol-5-yl)imidazo[1,5-b]pyridazin-2-yl)-3-methylmorpholine |
| 36 | 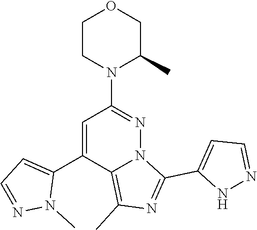 [SMILES: Cc1nc(-c2ccn[nH]2)n2nc(N3CCOC[C@H]3C)cc(-c3ccnn3C)c12] |
| | (R)-3-methyl-4-(5-methyl-4-(1-methyl-1H-pyrazol-5-yl)-7-(1H- |
| | pyrazol-5-yl)imidazo[1,5-b]pyridazin-2-yl)morpholine |
| 37 | 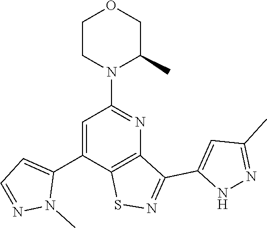 [SMILES: Cc1cc(-c2nsc3c(-c4ccnn4C)cc(N4CCOC[C@H]4C)nc23)[nH]n1] |
| | (R)-3-methyl-4-(7-(1-methyl-1H-pyrazol-5-yl)-3-(3-methyl-1H- |
| | pyrazol-5-yl)isothiazolo[4,5-b]pyridin-5-yl)morpholine |
| 38 | 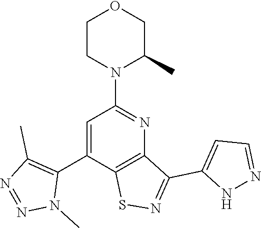 [SMILES: Cc1nnn(C)c1-c1cc(N2CCOC[C@H]2C)nc2c(-c3ccn[nH]3)nsc12] |
| | (R)-4-(7-(1,4-dimethyl-1H-1,2,3-triazol-5-yl)-3-(1H-pyrazol-5- |
| | yl)isothiazolo[4,5-b]pyridin-5-yl)-3-methylmorpholine |
| 39 | 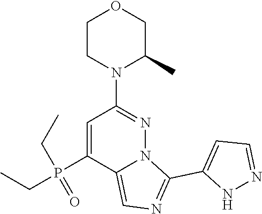 [SMILES: CCP(=O)(CC)c1cc(N2CCOC[C@H]2C)nn2c(-c3ccn[nH]3)ncc12] |
| | (3R)-4-[4-(diethylphosphoryl)-7-(1H-pyrazol-5-yl)imidazo [1,5- |
| | b]pyridazin-2-yl]-3-methylmorpholine |
| 40 | 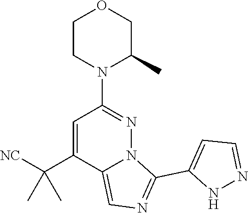 [SMILES: C[C@@H]1COCCN1c1cc(C(C)(C)C#N)c2cnc(-c3ccn[nH]3)n2n1] |
| | (R)-2-methyl-2-(2-(3-methylmorpholino)-7-(1H-pyrazol-5- |
| | yl)imidazo[1,5-b]pyridazin-4-yl)propanenitrile |
| 41 | 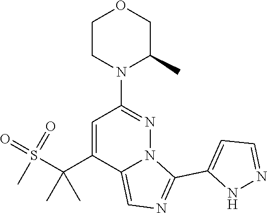 [SMILES: C[C@@H]1COCCN1c1cc(C(C)(C)S(C)(=O)=O)c2cnc(-c3ccn[nH]3)n2n1] |
| | (3R)-4-[4-(2-methanesulfonylpropan-2-yl)-7-(1H-pyrazol -5- |
| | yl)imidazo[1,5-b]pyridazin-2-yl]-3-methylmorpholine |
| 42 | 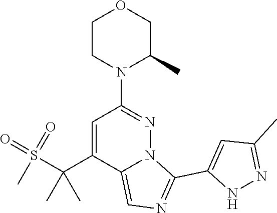 [SMILES: Cc1cc(-c2ncc3c(C(C)(C)S(C)(=O)=O)cc(N4CCOC[C@H]4C)nn23)[nH]n1] |
| | (R)-3-methyl-4-(7-(3-methyl-1H-pyrazol-5-yl)-4-(2-(methyl |
| | sulfonyl)propan-2-yl)imidazo[1,5-b]pyridazin-2-yl)morpholine |
| 43 | 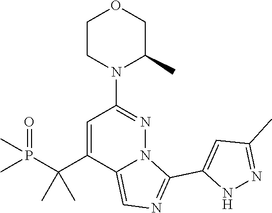 |
| | (R)-dimethyl(2-(7-(3-methyl-1H-pyrazol-5-yl)-2-(3- |
| | methylmorpholino)imidazo[1,5-b]pyridazin-4-yl)propan-2- |
| | yl)phosphine oxide |
| 44 | 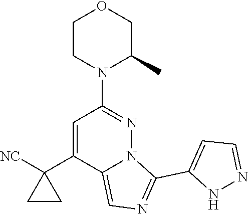 [SMILES: C[C@@H]1COCCN1c1cc(C2(C#N)CC2)c2cnc(-c3ccn[nH]3)n2n1] |
| | (R)-1-(2-(3-methylmorpholino)-7-(1H-pyrazol-5-yl)imidazo[1,5-b] |
| | pyridazin-4-yl)cyclopropane-1-carbonitrile |
| 45 | 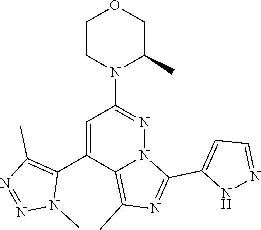 [SMILES: Cc1nnn(C)c1-c1cc(N2CCOC[C@H]2C)nn2c(-c3ccn[nH]3)nc(C)c12] |
| | (3R)-4-[4-(dimethyl-1H-1,2,3-triazol-5-yl)-5-methyl-7-(1H- |
| | pyrazol-5-yl)imidazo[1,5-b]pyridazin-2-yl]-3-methylmorpholine |
| 46 | 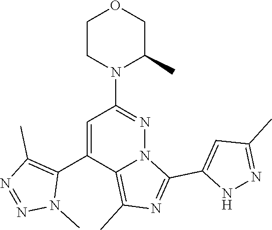 [SMILES: Cc1cc(-c2nc(C)c3c(-c4c(C)nnn4C)cc(N4CCOC[C@H]4C)nn23)[nH]n1] |
| | (3R)-4-[4-(dimethyl-1H-1,2,3-triazol-5-yl)-5-methyl-7-(3-methyl- |
| | 1H-pyrazol-5-yl)imidazo[1,5-b]pyridazin-2-yl]-3-methylmorpholine |
| 47 | 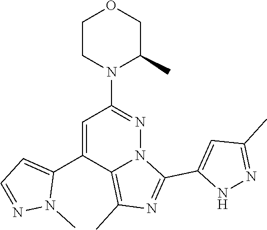 [SMILES: Cc1cc(-c2nc(C)c3c(-c4ccnn4C)cc(N4CCOC[C@H]4C)nn23)[nH]n1] |
| | (R)-3-methyl-4-(5-methyl-4-(1-methyl-1H-pyrazol-5-yl)-7-(3- |
| | methyl-1H-pyrazol-5-yl)imidazo[1,5-b]pyridazin-2-yl)morpholine |
| 48 | 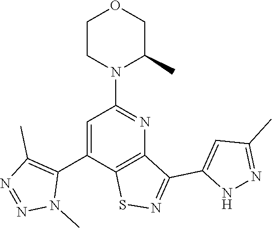 [SMILES: Cc1cc(-c2nsc3c(-c4c(C)nnn4C)cc(N4CCOC[C@H]4C)nc23)[nH]n1] |
| | (R)-4-(7-(1,4-dimethyl-1H-1,2,3-triazol-5-yl)-3-(3-methyl-1H- |
| | pyrazol-5-yl)isothiazolo[4,5-b]pyridin-5-yl)-3-methylmorpholine |
| 49 | 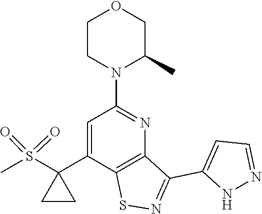 [SMILES: C[C@@H]1COCCN1c1cc(C2(S(C)(=O)=O)CC2)c2snc(-c3ccn[nH]3)c2n1] |
| | (R)-3-methyl-4-(7-(1-(methylsulfony])cyclopropyl)-3-(1H- |
| | pyrazol-5-yl)isothiazolo[4,5-b]pyridin-5-yl)morpholine |
| 50 | 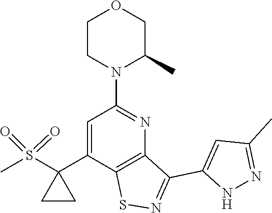 |
| | (R)-3-methyl-4-(3-(3-methyl-1H-pyrazol-5-yl)-7-(1- |
| | (methylsulfonyl)cyclopropyl)isothiazolo[4,5-b]pyridin-5- |
| | yl)morpholine |
| 51 | 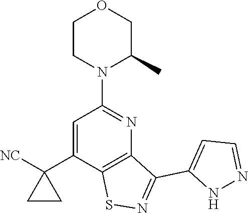 [SMILES: C[C@@H]1COCCN1c1cc(C2(C#N)CC2)c2snc(-c3ccn[nH]3)c2n1] |
| | (R)-1-(5-(3-methylmorpholino)-3-(1H-pyrazol-5-yl)isothiazolo |
| | [4,5-b] pyridin-7-yl)cyclopropane-1-carbonitrile |
| 52 | 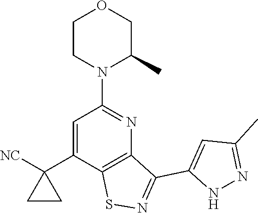 [SMILES: Cc1cc(-c2nsc3c(C4(C#N)CC4)cc(N4CCOC[C@H]4C)nc23)[nH]n1] |
| | (R)-1-(3-(3-methyl-1H-pyrazol-5-yl)-5-(3- |
| | methylmorpholino)isothiazolo [4,5-b]pyridin-7-yl)cyclopropane-1- |
| | carbonitrile |
| 53 | 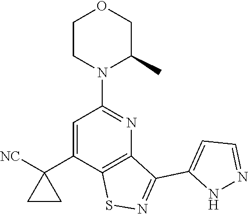 [SMILES: C[C@@H]1COCCN1c1cc(C2(C#N)CC2)c2snc(-c3ccn[nH]3)c2n1] |
| | (R)-2-methyl-2-(5-(3-methylmorpholino)-3-(1H-pyrazol-5-yl) |
| | isothiazolo [4,5-b]pyridin-7-yl)propanenitrile |
| 54 | 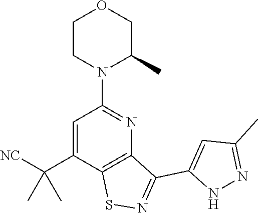 [SMILES: Cc1cc(-c2nsc3c(C(C)(C)C#N)cc(N4CCOC[C@H]4C)nc23)[nH]n1] |
| | (R)-2-methyl-2-(3-(3-methyl-1H-pyrazol-5-yl)-5-(3- |
| | methylmorpholino)isothiazolo[4,5-b]pyridin-7-yl)propanenitrile |
| 55 | 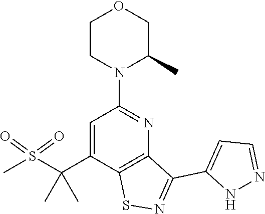 [SMILES: C[C@@H]1COCCN1c1cc(C(C)(C)S(C)(=O)=O)c2snc(-c3ccn[nH]3)c2n1] |
| | (R)-3-methyl-4-(7-(2-(methylsulfony])propan-2-yl)-3-(1H-pyrazol- |
| | 5-yl)isothiazolo[4,5-b]pyridin-5-yl)morpholine |
| 56 | 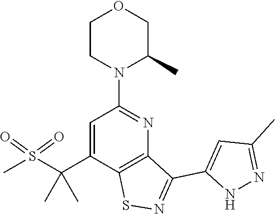 |
| | (R)-3-methyl-4-(3-(3-methyl-1H-pyrazol-5-yl)-7-(2- |
| | (methylsulfonyl)propan-2-yl)isothiazolo[4,5-b]pyridin-5- |
| | yl)morpholine |
| 57 | 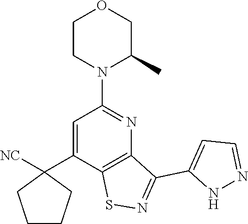 [SMILES: C[C@@H]1COCCN1c1cc(C2(C#N)CCCC2)c2snc(-c3ccn[nH]3)c2n1] |
| | (R)-1-(5-(3-methylmorpholino)-3-(1H-pyrazol-5-yl)isothiazolo |
| | [4,5-b]pyridin-7-yl)cyclopentane-1-carbonitrile |
| 58 | 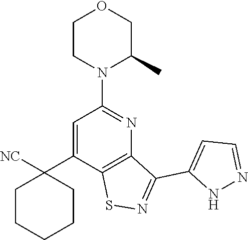 [SMILES: C[C@@H]1COCCN1c1cc(C2(C#N)CCCCC2)c2snc(-c3ccn[nH]3)c2n1] |
| | (R)-1-(5-(3-methylmorpholino)-3-(1H-pyrazol-5-yl)isothiazolo[4,5- |
| | b]pyridin-7-yl)cyclohexane-1-carbonitrile |
| 59 | 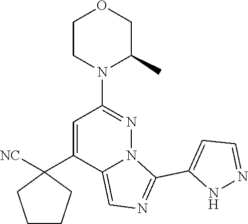 [SMILES: C[C@@H]1COCCN1c1cc(C2(C#N)CCCC2)c2cnc(-c3ccn[nH]3)n2n1] |
| | (R)-1-(2-(3-methylmorpholino)-7-(1H-pyrazol-5-yl)imidazo[1,5- |
| | b]pyridazin-4-yl)cyclopentane-1-carbonitrile |
| 60 | 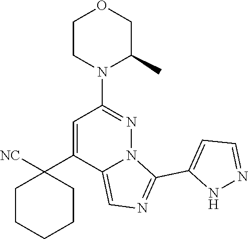 [SMILES: C[C@@H]1COCCN1c1cc(C2(C#N)CCCCC2)c2cnc(-c3ccn[nH]3)n2n1] |
| | (R)-1-(2-(3-methylmorpholino)-7-(1H-pyrazol-5-yl)imidazo[1,5- |
| | b]pyridazin-4-yl)cyclohexane-1-carbonitrile |
| 61 | 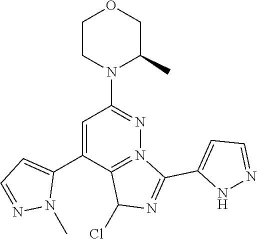 |
| | (3R)-4-[5-chloro-4-(1-methyl-1H-pyrazol-5-yl)-7-(1H-pyrazol-5- |
| | yl)imidazo[1,5-b]pyridazin-2-yl]-3-methylmorpholine |
| 62 | 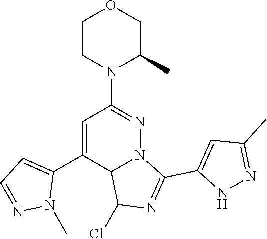 [SMILES: Cc1cc(C2=NC(Cl)C3C(c4ccnn4C)=CC(N4CCOC[C@H]4C)=NN23)[nH]n1] |
| | (R)-4-(5-chloro-4-(1-methyl-1H-pyrazol-5-yl)-7-(3-methyl-1H- |
| | pyrazol-5-yl)imidazo[1,5-b]pyridazin-2-yl)-3-methylmorpholine |
| 63 | 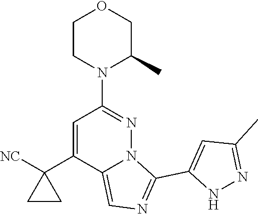 [SMILES: Cc1cc(-c2ncc3c(C4(C#N)CC4)cc(N4CCOC[C@H]4C)nn23)[nH]n1] |
| | (R)-1-(7-(3-methyl-1H-pyrazol-5-yl)-2-(3- |
| | methylmorpholino)imidazo[1,5-b]pyridazin-4-yl)cyclopropane-1- |
| | carbonitrile |
| 64 | 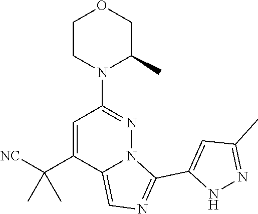 [SMILES: Cc1cc(-c2ncc3c(C(C)(C)C#N)cc(N4CCOC[C@H]4C)nn23)[nH]n1] |
| | (R)-2-methyl-2-(7-(3-methyl-1H-pyrazol-5-yl)-2-(3- |
| | methylmorpholino) imidazo[1,5-b]pyridazin-4-yl)propanenitrile |
| 65 | 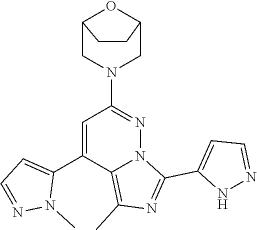 [SMILES: Cc1nc(-c2ccn[nH]2)n2nc(N3CC4CCC(C3)O4)cc(-c3ccnn3C)c12] |
| | 4-(5-methyl-4-(1-methyl-1H-pyrazol-5-yl)-7-(1H-pyrazol-5- |
| | yl)imidazo[1,5-b]pyridazin-2-yl)morpholine |
| 66 | 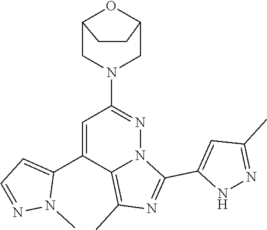 [SMILES: Cc1cc(-c2nc(C)c3c(-c4ccnn4C)cc(N4CC5CCC(C4)O5)nn23)[nH]n1] |
| | 4-(5-methyl-4-(1-methyl-1H-pyrazol-5-yl)-7-(3-methyl-1H- |
| | pyrazol-5-yl)imidazo[1,5-b]pyridazin-2-yl)morpholine |
| 67 | 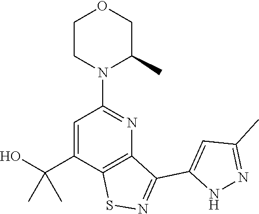 [SMILES: Cc1cc(-c2nsc3c(C(C)(C)O)cc(N4CCOC[C@H]4C)nc23)[nH]n1] |
| | (R)-2-(3-(3-methyl-1H-pyrazol-5-yl)-5-(3- |
| | methylmorpholino)isothiazolo[4,5-b]pyridin-7-yl)propan-2-ol |
| 68 | 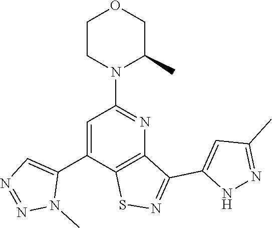 [SMILES: Cc1cc(-c2nsc3c(-c4cnnn4C)cc(N4CCOC[C@H]4C)nc23)[nH]n1] |
| | (R)-3-methyl-4-(7-(1-methyl-1H-1,2,3-triazol-5-yl)-3-(3-methyl- |
| | 1H-pyrazol-5-yl)isothiazolo[4,5-b]pyridin-5-yl)morpholine |
| 69 | 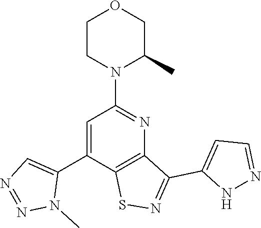 [SMILES: C[C@@H]1COCCN1c1cc(-c2cnnn2C)c2snc(-c3ccn[nH]3)c2n1] |
| | (R)-3-methyl-4-(7-(1-methyl-1H-1,2,3-triazol-5-yl)-3-(1H- |
| | pyrazol-5-yl)isothiazolo[4,5-b]pyridin-5-yl)morpholine |
| 70 | 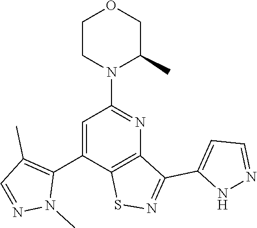 [SMILES: Cc1cnn(C)c1-c1cc(N2CCOC[C@H]2C)nc2c(-c3ccn[nH]3)nsc12] |
| | (R)-4-(7-(1,4-dimethyl-1H-pyrazol-5-yl)-3-(1H-pyrazol-5- |
| | yl)isothiazolo[4,5-b]pyridin-5-yl)-3-methylmorpholine |
| 71 | 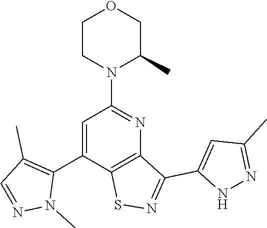 [SMILES: Cc1cc(-c2nsc3c(-c4c(C)cnn4C)cc(N4CCOC[C@H]4C)nc23)[nH]n1] |
| | (R)-4-(7-(1,4-dimethyl-1H-pyrazol-5-yl)-3-(3-methyl-1H-pyrazol-5- |
| | yl)isothiazolo[4,5-b]pyridin-5-yl)-3-methylmorpholine |
| 72 | 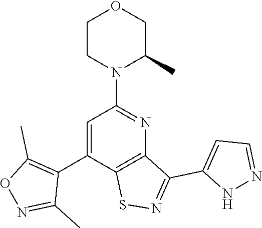 [SMILES: Cc1noc(C)c1-c1cc(N2CCOC[C@H]2C)nc2c(-c3ccn[nH]3)nsc12] |
| | (R)-4-(7-(3,5-dimethylisoxazol-4-yl)-3-(1H-pyrazol-5- |
| | yl)isothiazolo[4,5-b]pyridin-5-yl)-3-methylmorpholine |
| 73 | 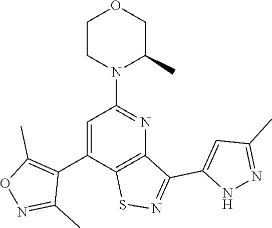 [SMILES: Cc1cc(-c2nsc3c(-c4c(C)noc4C)cc(N4CCOC[C@H]4C)nc23)[nH]n1] |
| | (R)-4-(7-(3,5-dimethylisoxazol-4-yl)-3-(3-methyl-1H-pyrazol-5- |
| | yl)isothiazolo[4,5-b]pyridin-5-yl)-3-methylmorpholine |
| 74 | 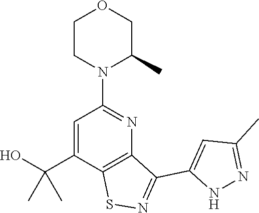 [SMILES: Cc1cc(-c2nsc3c(C(C)(C)O)cc(N4CCOC[C@H]4C)nc23)[nH]n1] |
| | (R)-2-(3-(3-methyl-1H-pyrazol-5-yl)-5-(3- |
| | methylmorpholino)isothiazolo[4,5-b]pyridin-7-yl)propan-2-ol |
| 75 | 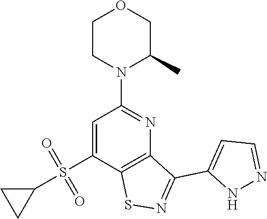 [SMILES: C[C@@H]1COCCN1c1cc(S(=O)(=O)C2CC2)c2snc(-c3ccn[nH]3)c2n1] |
| | (R)-4-(7-(cyclopropylsulfonyl)-3-(1H-pyrazol-5-yl)isothiazolo |
| | [4,5-b]pyridin-5-yl)-3-methylmorpholine |
| 76 | 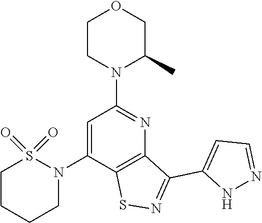 [SMILES: C[C@@H]1COCCN1c1cc(N2CCCCS2(=O)=O)c2snc(-c3ccn[nH]3)c2n1] |
| | (R)-2-(5-(3-methylmorpholino)-3-(1H-pyrazol-5-yl)isothiazolo |
| | [4,5-b]pyridin-7-yl)-1,2-thiazinane 1,1-dioxide |
| 77 | 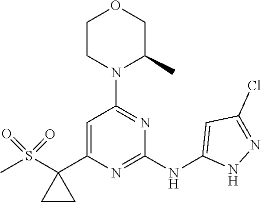 [SMILES: C[C@@H]1COCCN1c1cc(C2(S(C)(=O)=O)CC2)nc(Nc2cc(Cl)n[nH]2)n1] |
| | (R)-N-(3-chloro-1H-pyrazol-5-yl)-4-(3-methylmorpholino)-6-(1- |
| | (methylsulfonyl)cyclopropyl)pyrimidin-2-amine |
| 78 | 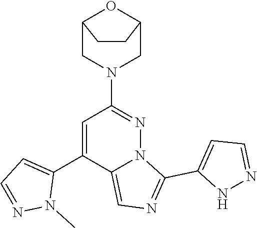 [SMILES: Cn1nccc1-c1cc(N2CC3CCC(C2)O3)nn2c(-c3ccn[nH]3)ncc12] |
| | (1R,5S)-3-(4-(1-methyl-1H-pyrazol-5-yl)-7-(1H-pyrazol-5- |
| | yl)imidazo[1,5-b]pyridazin-2-yl)-8-oxa-3-azabicyclo[3.2.1]octane |
| 79 | 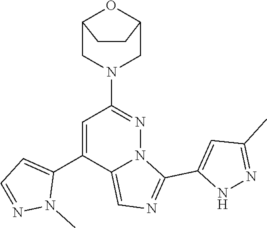 [SMILES: Cc1cc(-c2ncc3c(-c4ccnn4C)cc(N4CC5CCC(C4)O5)nn23)[nH]n1] |
| | (1R,5S)-3-(4-(1-methyl-1H-pyrazol-5-yl)-7-(3-methyl-1H- |
| | pyrazol-5-yl)imidazo[1,5-b]pyridazin-2-yl)-8-oxa-3- |
| | azabicyclo[3.2.1]octane |
| 80 | 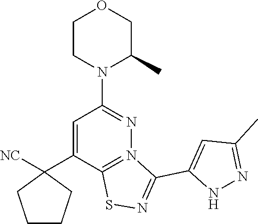 |
| | (R)-1-(3-(3-methyl-1H-pyrazol-5-yl)-5-(3- |
| | methylmorpholino)isothiazolo[4,5-b]pyridin-7-yl)cyclopentane-1- |
| | carbonitrile |
| 81 | 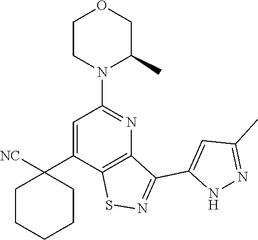 [SMILES: Cc1cc(-c2nsc3c(C4(C#N)CCCCC4)cc(N4CCOC[C@H]4C)nc23)[nH]n1] |
| | (R)-1-(3-(3-methyl-1H-pyrazol-5-yl)-5-(3- |
| | methylmorpholino)isothiazolo[4,5-b]pyridin-7-yl)cyclohexane-1- |
| | carbonitrile |
| 82 | 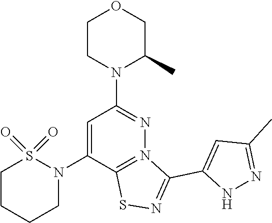 |
| 83 | 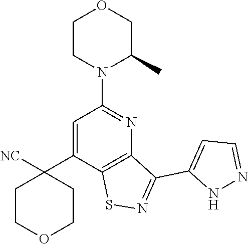 [SMILES: C[C@@H]1COCCN1c1cc(C2(C#N)CCOCC2)c2snc(-c3ccn[nH]3)c2n1] |
| | (R)-4-(5-(3-methylmorpholino)-3-(1H-pyrazol-5-yl)isothiazolo |
| | [4,5-b]pyridin-7-yl)tetrahydro-2H-pyran-4-carbonitrile |
| 84 | 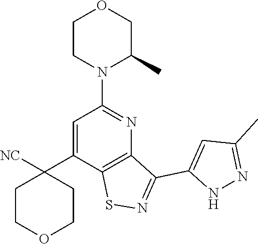 [SMILES: Cc1cc(-c2nsc3c(C4(C#N)CCOCC4)cc(N4CCOC[C@H]4C)nc23)[nH]n1] |
| | (R)-4-(3-(3-methyl-1H-pyrazol-5-yl)-5-(3- |
| | methylmorpholino)isothiazolo[4,5-b]pyridin-7-yl)tetrahydro-2H- |
| | pyran-4-carbonitrile |
| 85 | 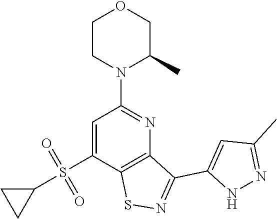 [SMILES: Cc1cc(-c2nsc3c(S(=O)(=O)C4CC4)cc(N4CCOC[C@H]4C)nc23)[nH]n1] |
| | (R)-4-(7-(cyclopropylsulfonyl)-3-(3-methyl-1H-pyrazol-5- |
| | yl)isothiazolo[4,5-b]pyridin-5-yl)-3-methylmorpholine |
| 86 | 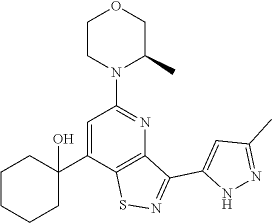 [SMILES: Cc1cc(-c2nsc3c(C4(O)CCCCC4)cc(N4CCOC[C@H]4C)nc23)[nH]n1] |
| | (R)-1-(3-(3-methyl-1H-pyrazol-5-yl)-5-(3- |
| | methylmorpholino)isothiazolo[4,5-b]pyridin-7-yl) |
| | cyclohexan-1-ol |
| 87 | 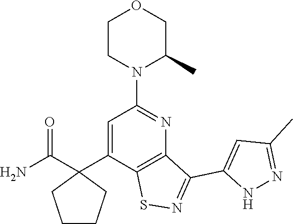 [SMILES: Cc1cc(-c2nsc3c(C4(C(N)=O)CCCC4)cc(N4CCOC[C@H]4C)nc23)[nH]n1] |
| | (R)-1-(3-(3-methyl-1H-pyrazol-5-yl)-5-(3- |
| | methylmorpholino)isothiazolo[4,5-b]pyridin-7-yl)cyclopentane-1- |
| | carboxamide |
| 88 | 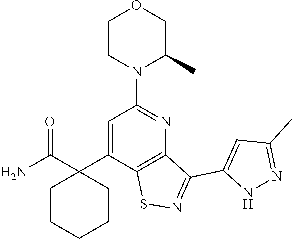 [SMILES: Cc1cc(-c2nsc3c(C4(C(N)=O)CCCCC4)cc(N4CCOC[C@H]4C)nc23)[nH]n1] |
| | (R)-1-(3-(3-methyl-1H-pyrazol-5-yl)-5-(3- |
| | methylmorpholino)isothiazolo[4,5-b]pyridin-7-yl)cyclohexane-1- |
| | carboxamide |
| 89 | 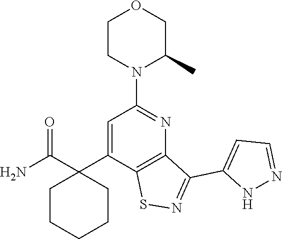 [SMILES: C[C@@H]1COCCN1c1cc(C2(C(N)=O)CCCCC2)c2snc(-c3ccn[nH]3)c2n1] |
| | (R)-1-(5-(3-methylmorpholino)-3-(1H-pyrazol-5-yl)isothiazolo |
| | [4,5-b]pyridin-7-yl)cyclohexane-1-carboxamide |
| 90 | 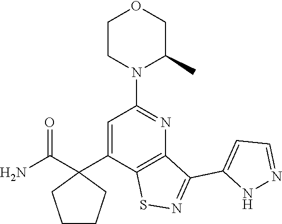 [SMILES: C[C@@H]1COCCN1c1cc(C2(C(N)=O)CCCC2)c2snc(-c3ccn[nH]3)c2n1] |
| | (R)-1-(5-(3-methylmorpholino)-3-(1H-pyrazol-5-yl)isothiazolo |
| | [4,5-b]pyridin-7-yl)cyclopentane-1-carboxamide |
| 91 | 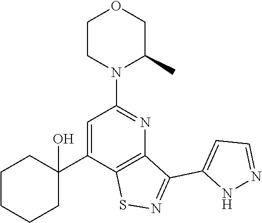 [SMILES: C[C@@H]1COCCN1c1cc(C2(O)CCCCC2)c2snc(-c3ccn[nH]3)c2n1] |
| | (R)-1-(5-(3-methylmorpholino)-3-(1H-pyrazol-5-yl)isothiazolo |
| | [4,5-b]pyridin-7-yl)cyclohexan-1-ol |
| 92 | 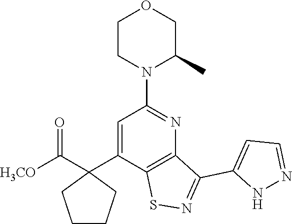 [SMILES: COC(=O)C1(c2cc(N3CCOC[C@H]3C)nc3c(-c4ccn[nH]4)nsc23)CCCC1] |
| | methyl (R)-1-(5-(3-methylmorpholino)-3-(1H-pyrazol-5- |
| | yl)isothiazolo[4,5-b]pyridin-7-yl)cyclopentane-1-carboxylate |
| 93 | 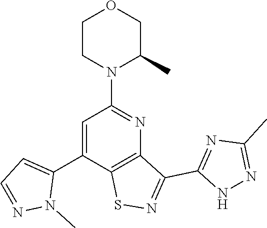 [SMILES: Cc1n[nH]c(-c2nsc3c(-c4ccnn4C)cc(N4CCOC[C@H]4C)nc23)n1] |
| | (R)-3-methyl-4-(3-(3-methyl-1H-1,2,4-triazol-5-yl)-7-(1-methyl- |
| | 1H-pyrazol-5-yl)isothiazolo[4,5-b]pyridin-5-yl)morpholine |
| 94 | 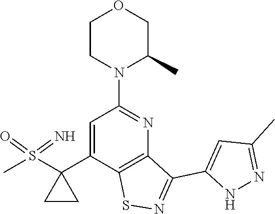 |
| | imino(methyl)(1-(3-(3-methyl-1H-pyrazol-5-yl)-5-((R)-3- |
| | methylmorpholino)isothiazolo[4,5-b]pyridin-7-yl)cyclopropyl)- |
| | λ⁶-sulfanone |
| 95 | 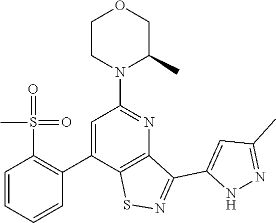 |
| | (R)-3-methyl-4-(3-(3-methyl-1H-pyrazol-5-yl)-7-(2- |
| | (methylsulfonyl)phenyl)isothiazolo[4,5-b]pyridin-5-yl)morpholine |
| 96 | 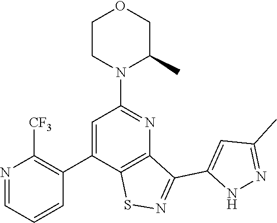 [SMILES: Cc1cc(-c2nsc3c(-c4cccnc4C(F)(F)F)cc(N4CCOC[C@H]4C)nc23)[nH]n1] |
| | (R)-3-methyl-4-(3-(3-methyl-1H-pyrazol-5-y])-7-(2- |
| | (trifluoromethyl)pyridin-3-yl)isothiazolo[4,5-b]pyridin-5- |
| | yl)morpholine |
| 97 | 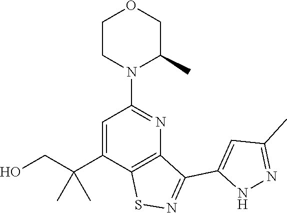 [SMILES: Cc1cc(-c2nsc3c(C(C)(C)CO)cc(N4CCOC[C@H]4C)nc23)[nH]n1] |
| | (R)-2-methyl-2-(3-(3-methyl-1H-pyrazol-5-yl)-5-(3- |
| | methylmorpholino)isothiazolo[4,5-b]pyridin-7-yl)propan-1-ol |
| 98 | 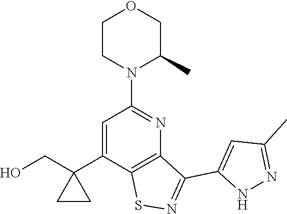 [SMILES: Cc1cc(-c2nsc3c(C4(CO)CC4)cc(N4CCOC[C@H]4C)nc23)[nH]n1] |
| | (R)-(1-(3-(3-methyl-1H-pyrazol-5-yl)-5-(3- |
| | methylmorpholino)isothiazolo[4,5-b]pyridin-7- |
| | yl)cyclopropyl)methanol |
| 99 | 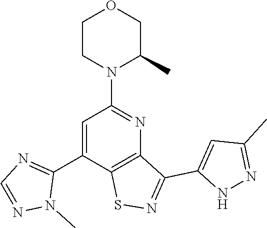 [SMILES: Cc1cc(-c2nsc3c(-c4ncnn4C)cc(N4CCOC[C@H]4C)nc23)[nH]n1] |
| | (R)-3-methyl-4-(7-(1-methyl-1H-1,2,3-triazol-5-yl)-3-(3-methyl- |
| | 1H-pyrazol-5-yl)isothiazolo[4,5-b]pyridin-5-yl)morpholine |
| 100 | 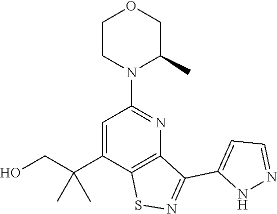 [SMILES: C[C@@H]1COCCN1c1cc(C(C)(C)CO)c2snc(-c3ccn[nH]3)c2n1] |
| | (R)-2-methyl-2-(5-(3-methylmorpholino)-3-(1H-pyrazol-5- |
| | yl)isothiazolo[4,5-b]pyridin-7-yl)propan-1-ol |
| 101 | 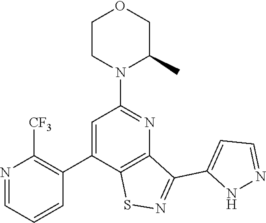 [SMILES: C[C@@H]1COCCN1c1cc(-c2cccnc2C(F)(F)F)c2snc(-c3ccn[nH]3)c2n1] |
| | (R)-4-(3-(1H-pyrazol-5-yl)-7-(2-(trifluoromethyl)pyridin-3- |
| | yl )isothiazolo[4,5-b]pyridin-5-yl)-3-methylmorpholine |
| 102 | 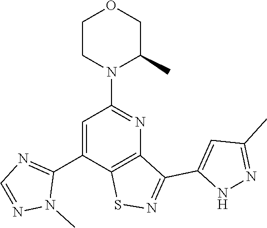 [SMILES: Cc1cc(-c2nsc3c(-c4ncnn4C)cc(N4CCOC[C@H]4C)nc23)[nH]n1] |
| | (R)-3-methyl-4-(7-(1-methyl-1H-1,2,4-triazol-5-yl)-3-(3-methyl- |
| | 1H-pyrazol-5-yl)isothiazolo[4,5-b]pyridin-5-yl)morpholine |
| 103 | 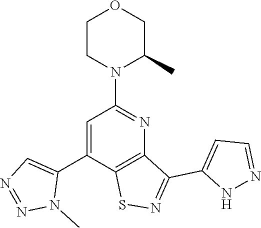 [SMILES: C[C@@H]1COCCN1c1cc(-c2cnnn2C)c2snc(-c3ccn[nH]3)c2n1] |
| | (R)-3-methyl-4-(7-(1-methyl-1H-1,2,3-triazol-5-yl)-3-(1H- |
| | pyrazol-5-yl)isothiazolo[4,5-b]pyridin-5-yl)morpholine |
| 104 | 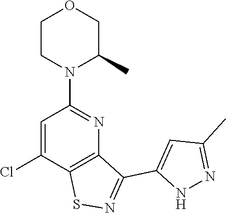 [SMILES: Cc1cc(-c2nsc3c(Cl)cc(N4CCOC[C@H]4C)nc23)[nH]n1] |
| | (R)-4-(7-chloro-3-(3-methyl-1H-pyrazol-5-yl)isothiazolo[4,5- |
| | b]pyridin-5-yl)-3-methylmorpholine |
| 105 | 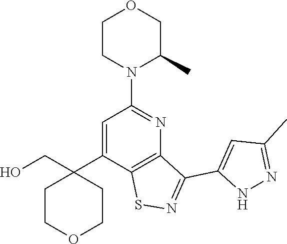 [SMILES: Cc1cc(-c2nsc3c(C4(CO)CCOCC4)cc(N4CCOC[C@H]4C)nc23)[nH]n1] |
| | (R)-(4-(3-(3-methyl-1H-pyrazol-5-yl)-5-(3- |
| | methylmorpholino)isothiazolo[4,5-b]pyridin-7-yl)tetrahydro-2H- |
| | pyran-4-yl)methanol |
| 106 | 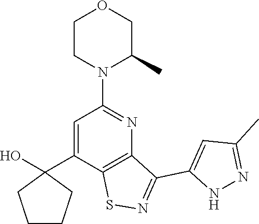 [SMILES: Cc1cc(-c2nsc3c(C4(O)CCCC4)cc(N4CCOC[C@H]4C)nc23)[nH]n1] |
| | (R)-1-(3-(3-methyl-1H-pyrazol-5-yl)-5-(3- |
| | methylmorpholino)isothiazolo[4,5-b]pyridin-7-yl)cyclopentan-1-ol |
| 107 | 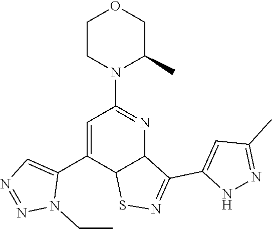 [SMILES: CCn1nncc1C1=CC(N2CCOC[C@H]2C)=NC2C(c3cc(C)n[nH]3)=NSC12] |
| | (R)-4-(7-(1-ethyl-1H-1,2,3-triazol-5-yl)-3-(3-methyl-1H-pyrazol- |
| | 5-yl)isothiazolo[4,5-b]pyridin-5-yl)-3-methylmorpholine |
| 108 | 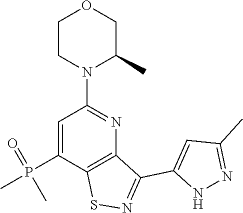 |
| | (R)-dimethyl(3-(3-methyl-1H-pyrazol-5-yl)-5-(3- |
| | methylmorpholino)isothiazolo[4,5-b]pyridin-7-yl)phosphine oxide |
| 109 | 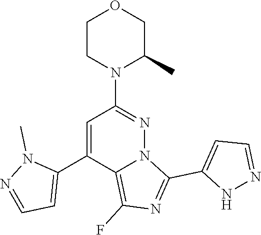 [SMILES: C[C@@H]1COCCN1c1cc(-c2ccnn2C)c2c(F)nc(-c3ccn[nH]3)n2n1] |
| | (R)-4-(5-fluoro-4-(1-methyl-1H-pyrazol-5-yl)-7-(1H-pyrazol-5- |
| | yl)imidazo[1,5-b]pyridazin-2-yl)-3-methylmorpholine |

Compounds provided herein are described with reference to both generic formulae and specific compounds. In addition, the compounds of the present disclosure may exist in a number of different forms or derivatives, including but not limited to prodrugs, soft drugs, active metabolic derivatives (active metabolites), and their pharmaceutically acceptable salts, all within the scope of the present disclosure.

As used herein, the term “prodrugs” refers to compounds or pharmaceutically acceptable salts thereof which, when metabolized under physiological conditions or when converted by solvolysis, yield the desired active compound. Prodrugs include, without limitation, esters, amides, carbamates, carbonates, ureides, solvates, or hydrates of the active compound. Typically, the prodrug is inactive, or less active than the active compound, but may provide one or more advantageous handling, administration, and/or metabolic properties. For example, some prodrugs are esters of the active compound; during metabolysis, the ester group is cleaved to yield the active drug. Also, some prodrugs are activated enzymatically to yield the active compound, or a compound which, upon further chemical reaction, yields the active compound. Prodrugs may proceed from prodrug form to active form in a single step or may have one or more intermediate forms which may themselves have activity or may be inactive. Preparation and use of prodrugs is discussed in T. Higuchi and V.

Stella, “Pro-drugs as Novel Delivery Systems”, Vol. 14 of the A.C.S. Symposium Series, in Bioreversible Carriers in Drug Design, ed. Edward B. Roche, American Pharmaceutical Association and Pergamon Press, 1987; in Prodrugs: Challenges and Rewards, ed. V. Stella, R. Borchardt, M. Hageman, R. Oliyai, H. Maag, J. Tilley, Springer-Verlag New York, 2007, all of which are hereby incorporated by reference in their entirety.

As used herein, the term “soft drug” refers to compounds that exert a pharmacological effect but break down to inactive metabolites degradants so that the activity is of limited time. See, for example, “Soft drugs: Principles and methods for the design of safe drugs”, Nicholas Bodor, Medicinal Research Reviews, Vol. 4, No. 4, 449-469, 1984, which is hereby incorporated by reference in its entirety.

As used herein, the term “metabolite”, e.g., active metabolite overlaps with prodrug as described above. Thus, such metabolites are pharmacologically active compounds or compounds that further metabolize to pharmacologically active compounds that are derivatives resulting from metabolic process in the body of a subject. For example, such metabolites may result from oxidation, reduction, hydrolysis, amidation, deamidation, esterification, deesterification, enzymatic cleavage, and the like, of the administered compound or salt or prodrug. Of these, active metabolites are such pharmacologically active derivative compounds. For prodrugs, the prodrug compound is generally inactive or of lower activity than the metabolic product. For active metabolites, the parent compound may be either an active compound or may be an inactive prodrug.

Prodrugs and active metabolites may be identified using routine techniques know in the art. See, e.g., Bertolini et al, 1997, J Med Chem 40:2011-2016; Shan et al., J Pharm Sci 86:756-757; Bagshawe, 1995, DrugDev Res 34:220-230; Wermuth, supra.

As used herein, the term “pharmaceutically acceptable” indicates that the substance or composition is compatible chemically and/or toxicologically, with the other ingredients comprising a formulation, and/or the subjects being treated therewith.

As used herein, the term “pharmaceutically acceptable salt”, unless otherwise indicated, includes salts that retain the biological effectiveness of the free acids and bases of the specified compound and that are not biologically or otherwise undesirable. Contemplated pharmaceutically acceptable salt forms include, but are not limited to, mono, bis, tris, tetrakis, and so on. Pharmaceutically acceptable salts are non-toxic in the amounts and concentrations at which they are administered. The preparation of such salts can facilitate the pharmacological use by altering the physical characteristics of a compound without preventing it from exerting its physiological effect. Useful alterations in physical properties include lowering the melting point to facilitate transmucosal administration and increasing the solubility to facilitate administering higher concentrations of the drug.

Pharmaceutically acceptable salts include acid addition salts such as those containing sulfate, chloride, hydrochloride, fumarate, maleate, phosphate, sulfamate, acetate, citrate, lactate, tartrate, methanesulfonate, ethanesulfonate, benzenesulfonate, p-toluenesulfonate, cyclohexylsulfamate and quinate. Pharmaceutically acceptable salts can be obtained from acids such as hydrochloric acid, maleic acid, sulfuric acid, phosphoric acid, sulfamic acid, acetic acid, citric acid, lactic acid, tartaric acid, malonic acid, methanesulfonic acid, ethanesulfonic acid, benzenesulfonic acid, p-toluenesulfonic acid, cyclohexylsulfamic acid, fumaric acid, and quinic acid.

Pharmaceutically acceptable salts also include basic addition salts such as those containing benzathine, chloroprocaine, choline, diethanolamine, ethanolamine, t-butylamine, ethylenediamine, meglumine, procaine, aluminum, calcium, lithium, magnesium, potassium, sodium, ammonium, alkylamine, and zinc, when acidic functional groups, such as carboxylic acid or phenol are present. For example, see Remington's Pharmaceutical Sciences, 19^{th} ed., Mack Publishing Co., Easton, PA, Vol. 2, p. 1457, 1995; “Handbook of Pharmaceutical Salts: Properties, Selection, and Use” by Stahl and Wermuth, Wiley-VCH, Weinheim, Germany, 2002. Such salts can be prepared using the appropriate corresponding bases.

Pharmaceutically acceptable salts can be prepared by standard techniques. For example, the free-base form of a compound can be dissolved in a suitable solvent, such as an aqueous or aqueous-alcohol solution containing the appropriate acid and then isolated by evaporating the solution. Thus, if the particular compound is a base, the desired pharmaceutically acceptable salt may be prepared by any suitable method available in the art, for example, treatment of the free base with an inorganic acid, such as hydrochloric acid, hydrobromic acid, sulfuric acid, nitric acid, phosphoric acid and the like, or with an organic acid, such as acetic acid, maleic acid, succinic acid, mandelic acid, fumaric acid, malonic acid, pyruvic acid, oxalic acid, glycolic acid, salicylic acid, a pyranosidyl acid, such as glucuronic acid or galacturonic acid, an alpha-hydroxy acid, such as citric acid or tartaric acid, an amino acid, such as aspartic acid or glutamic acid, an aromatic acid, such as benzoic acid or cinnamic acid, a sulfonic acid, such as p-toluenesulfonic acid or ethanesulfonic acid, or the like.

Similarly, if the particular compound is an acid, the desired pharmaceutically acceptable salt may be prepared by any suitable method, for example, treatment of the free acid with an inorganic or organic base, such as an amine (primary, secondary or tertiary), an alkali metal hydroxide or alkaline earth metal hydroxide, or the like.

Illustrative examples of suitable salts include organic salts derived from amino acids, such as L-glycine, L-lysine, and L-arginine, ammonia, primary, secondary, and tertiary amines, and cyclic amines, such as hydroxyethylpyrrolidine, piperidine, morpholine or piperazine, and inorganic salts derived from sodium, calcium, potassium, magnesium, manganese, iron, copper, zinc, aluminum and lithium.

It is also to be understood that the compounds of present disclosure can exist in unsolvated forms, solvated forms (e.g., hydrated forms), and solid forms (e.g., crystal or polymorphic forms), and the present disclosure is intended to encompass all such forms.

As used herein, the term “solvate” or “solvated form” refers to solvent addition forms that contain either stoichiometric or non-stoichiometric amounts of solvent. Some compounds have a tendency to trap a fixed molar ratio of solvent molecules in the crystalline solid state, thus forming a solvate. If the solvent is water the solvate formed is a hydrate; and if the solvent is alcohol, the solvate formed is an alcoholate. Hydrates are formed by the combination of one or more molecules of water with one molecule of the substance in which the water retains its molecular state as H₂O. Examples of solvents that form solvates include, but are not limited to, water, isopropanol, ethanol, methanol, DMSO, ethyl acetate, acetic acid, and ethanolamine.

As used herein, the terms “crystal form”, “crystalline form”, “polymorphic forms” and “polymorphs” can be used interchangeably, and mean crystal structures in which a compound (or a salt or solvate thereof) can crystallize in different crystal packing arrangements, all of which have the same elemental composition. Different crystal forms usually have different X-ray diffraction patterns, infrared spectral, melting points, density hardness, crystal shape, optical and electrical properties, stability and solubility. Recrystallization solvent, rate of crystallization, storage temperature, and other factors may cause one crystal form to dominate. Crystal polymorphs of the compounds can be prepared by crystallization under different conditions.

The compounds of present disclosure can comprise one or more asymmetric centers depending on substituent selection, and thus can exist in various stereoisomeric forms, e.g., enantiomers and/or diastereomers. For example, the compounds provided herein may have an asymmetric carbon center, and thus compounds provided herein may have either the (R) or (S) stereo-configuration at a carbon asymmetric center. Therefore, compounds of the present disclosure may be in the form of an individual enantiomer, diastereomer or geometric isomer, or may be in the form of a mixture of stereoisomers.

As used herein, the term “enantiomer” refers to two stereoisomers of a compound which are non-superimposable mirror images of one another. The term “diastereomer” refers to a pair of optical isomers which are not mirror images of one another. Diastereomers have different physical properties, e.g. melting points, boiling points, spectral properties, and reactivities.

Where a particular enantiomer is preferred, it may, in some embodiments be provided substantially free of the opposite enantiomer, and may also be referred to as “optically enriched”. “Optically enriched”, as used herein, means that the compound is made up of a significantly greater proportion of one enantiomer. In certain embodiments, the compound is made up of at least about 90% by weight of a preferred enantiomer. In other embodiments, the compound is made up of at least about 95%, 98%, or 99% by weight of a preferred enantiomer. Preferred enantiomers may be isolated from racemic mixtures by any method known to those skilled in the art, for example by chromatography or crystallization, by the use of stereochemically uniform starting materials for the synthesis or by stereoselective synthesis. Optionally a derivatization can be carried out before a separation of stereoisomers. The separation of a mixture of stereoisomers can be carried out at an intermediate step during the synthesis of a compound provided herein or it can be done on a final racemic product. Absolute stereochemistry may be determined by X-ray crystallography of crystalline products or crystalline intermediates which are derivatized, if necessary, with a reagent containing a stereogenic center of known configuration. Alternatively, absolute stereochemistry may be determined by Vibrational Circular Dichroism (VCD) spectroscopy analysis. See, for example, Jacques, et al., Enantiomers, Racemates and Resolutions (Wiley Interscience, New York, 1981); Wilen, S. H., et al., Tetrahedron 33:2725 (1977); Eliel, E. L. Stereochemistry of Carbon Compounds (McGraw-Hill, N Y, 1962); Wilen, S. H. Tables of Resolving Agents and Optical Resolutions p. 268 (E. L. Eliel, Ed., Univ. of Notre Dame Press, Notre Dame, IN 1972).

In some embodiments, mixtures of diastereomers, for example mixtures of diastereomers enriched with 51% or more of one of the diastereomers, including for example 60% or more, 70% or more, 80% or more, or 90% or more of one of the diastereomers are provided.

In some embodiments, compounds provided herein may have one or more double bonds that can exist as either the Z or E isomer, unless otherwise indicated. The present disclosure additionally encompasses the compounds as individual isomers substantially free of other isomers and alternatively, as mixtures of various isomers, e.g., racemic mixtures of enantiomers.

The compounds of the present disclosure may also exist in different tautomeric forms, and all such forms are embraced within the scope of the present disclosure. The term “tautomer” or “tautomeric form” refers to structural isomers of different energies which are interconvertible via a low energy barrier. For example, proton tautomers (also known as prototropic tautomers) include interconversions via migration of a proton, such as keto-enol, amide-imidic acid, lactam-lactim, imine-enamine isomerizations and annular forms where a proton can occupy two or more positions of a heterocyclic system (for example, 1H- and 3H-imidazole, 1H-, 2H- and 4H-1,2,4-triazole, 1H- and 2H-isoindole, and 1H- and 2H-pyrazole). Valence tautomers include interconversions by reorganization of some of the bonding electrons. Tautomers can be in equilibrium or sterically locked into one form by appropriate substitution. Compounds of the present disclosure identified by name or structure as one particular tautomeric form are intended to include other tautomeric forms unless otherwise specified.

The present disclosure is also intended to include all isotopes of atoms in the compounds. Isotopes of an atom include atoms having the same atomic number but different mass numbers. For example, unless otherwise specified, hydrogen, carbon, nitrogen, oxygen, phosphorous, sulfur, fluorine, chlorine, bromide or iodine in the compounds of present disclosure are meant to also include their isotopes, such as but not limited to ¹H, ²H, ³H, ¹¹C, ¹²C, ¹³C, ¹⁴C, ¹⁴N, ¹⁵N, ¹⁶O, ¹⁷O, ¹⁸O, ³¹P, ³²P, ³²S, ³³S, ³⁴S, ³⁶S, ¹⁷F, ¹⁸F, ¹⁹F, ³⁵Cl, ³⁷Cl, ⁷⁹Br, ⁸¹Br, ¹²⁴I, ¹²⁷I and ¹³¹I. In some embodiments, hydrogen includes protium, deuterium and tritium. In some embodiments, carbon includes ¹²C and ¹³C.

### Synthesis of Compounds

Synthesis of the compounds provided herein, including pharmaceutically acceptable salts thereof, are illustrated in the synthetic schemes in the examples. The compounds provided herein can be prepared using any known organic synthesis techniques and can be synthesized according to any of numerous possible synthetic routes, and thus these schemes are illustrative only and are not meant to limit other possible methods that can be used to prepare the compounds provided herein. Additionally, the steps in the Schemes are for better illustration and can be changed as appropriate. The embodiments of the compounds in examples were synthesized for the purposes of research and potentially submission to regulatory agencies.

The reactions for preparing compounds of the present disclosure can be carried out in suitable solvents, which can be readily selected by one skilled in the art of organic synthesis. Suitable solvents can be substantially non-reactive with the starting materials (reactants), the intermediates, or products at the temperatures at which the reactions are carried out, e.g. temperatures that can range from the solvent's freezing temperature to the solvent's boiling temperature. A given reaction can be carried out in one solvent or a mixture of more than one solvent. Depending on the particular reaction step, suitable solvents for a particular reaction step can be selected by one skilled in the art.

Preparation of compounds of the present disclosure can involve the protection and deprotection of various chemical groups. The need for protection and deprotection, and the selection of appropriate protecting groups, can be readily determined by one skilled in the art. The chemistry of protecting groups can be found, for example, in T. W. Greene and P. G. M. Wuts, Protective Groups in Organic Synthesis, 3rd Ed., Wiley & Sons, Inc., New York (1999), in P. Kocienski, Protecting Groups, Georg Thieme Verlag, 2003, and in Peter G. M. Wuts, Greene's Protective Groups in Organic Synthesis, 5^{th} Edition, Wiley, 2014, all of which are incorporated herein by reference in its entirety.

Reactions can be monitored according to any suitable method known in the art. For example, product formation can be monitored by spectroscopic means, such as nuclear magnetic resonance spectroscopy (e.g. ¹H or ¹³C), infrared spectroscopy, spectrophotometry (e.g. UV-visible), mass spectrometry, or by chromatographic methods such as high performance liquid chromatography (HPLC), liquid chromatography-mass spectroscopy (LCMS), or thin layer chromatography (TLC). Compounds can be purified by one skilled in the art by a variety of methods, including high performance liquid chromatography (HPLC) (“Preparative LC-MS Purification: Improved Compound Specific Method Optimization” Karl F. Blom, Brian Glass, Richard Sparks, Andrew P. Combs J. Combi. Chem. 2004, 6(6), 874-883, which is incorporated herein by reference in its entirety), and normal phase silica chromatography.

The known starting materials of the present disclosure can be synthesized by using or according to the known methods in the art, or can be purchased from commercial suppliers. Unless otherwise noted, analytical grade solvents and commercially available reagents were used without further purification.

Unless otherwise specified, the reactions of the present disclosure were all done under a positive pressure of nitrogen or argon or with a drying tube in anhydrous solvents, and the reaction flasks were typically fitted with rubber septa for the introduction of substrates and reagents via syringe. Glassware was oven dried and/or heat dried.

For illustrative purposes, the Examples section below shows synthetic route for preparing the compounds of the present disclosure as well as key intermediates. Those skilled in the art will appreciate that other synthetic routes may be used to synthesize the inventive compounds. Although specific starting materials and reagents are depicted, other starting materials and reagents can be easily substituted to provide a variety of derivatives and/or reaction conditions. In addition, many of the compounds prepared by the methods described below can be further modified in light of this disclosure using conventional chemistry well known to those skilled in the art.

### Pharmaceutical Compositions

In a further aspect, there is provided pharmaceutical compositions comprising one or more molecules or compounds of the present disclosure, or a pharmaceutically acceptable salt thereof.

In another aspect, there is provided pharmaceutical composition comprising one or more molecules or compounds of the present disclosure, or a pharmaceutically acceptable salt thereof, and at least one pharmaceutical acceptable excipient.

As used herein, the term “pharmaceutical composition” refers to a formulation containing the molecules or compounds of the present disclosure in a form suitable for administration to a subject.

As used herein, the term “pharmaceutically acceptable excipient” means an excipient that is useful in preparing a pharmaceutical composition that is generally safe, non-toxic and neither biologically nor otherwise undesirable, and includes excipient that is acceptable for veterinary use as well as human pharmaceutical use. A “pharmaceutically acceptable excipient” as used herein includes both one and more than one such excipient. The term “pharmaceutically acceptable excipient” also encompasses “pharmaceutically acceptable carrier” and “pharmaceutically acceptable diluent”.

The particular excipient used will depend upon the means and purpose for which the compounds of the present disclosure is being applied. Solvents are generally selected based on solvents recognized by persons skilled in the art as safe to be administered to a mammal including humans. In general, safe solvents are non-toxic aqueous solvents such as water and other non-toxic solvents that are soluble or miscible in water. Suitable aqueous solvents include water, ethanol, propylene glycol, polyethylene glycols (e.g., PEG 400, PEG 300), etc. and mixtures thereof.

In some embodiments, suitable excipients may include buffers such as phosphate, citrate and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN™, PLURONICS™ or polyethylene glycol (PEG).

In some embodiments, suitable excipients may include one or more stabilizing agents, surfactants, wetting agents, lubricating agents, emulsifiers, suspending agents, preservatives, antioxidants, opaquing agents, glidants, processing aids, colorants, sweeteners, perfuming agents, flavoring agents and other known additives to provide an elegant presentation of the drug (i.e., a compound of the present disclosure or pharmaceutical composition thereof) or aid in the manufacturing of the pharmaceutical product (i.e., medicament). The active pharmaceutical ingredients may also be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles and nanocapsules) or in macroemulsions. Such techniques are disclosed in Remington's Pharmaceutical Sciences 16th edition, Osol, A. Ed. (1980). A“liposome” is a small vesicle composed of various types of lipids, phospholipids and/or surfactant which is useful for delivery of a drug (such as the compounds disclosed herein and, optionally, a chemotherapeutic agent) to a mammal including humans. The components of the liposome are commonly arranged in a bilayer formation, similar to the lipid arrangement of biological membranes.

The pharmaceutical compositions provided herein can be in any form that allows for the composition to be administered to a subject, including, but not limited to a human, and formulated to be compatible with an intended route of administration.

A variety of routes are contemplated for the pharmaceutical compositions provided herein, and accordingly the pharmaceutical composition provided herein may be supplied in bulk or in unit dosage form depending on the intended administration route. For example, for oral, buccal, and sublingual administration, powders, suspensions, granules, tablets, pills, capsules, gelcaps, and caplets may be acceptable as solid dosage forms, and emulsions, syrups, elixirs, suspensions, and solutions may be acceptable as liquid dosage forms. For injection administration, emulsions and suspensions may be acceptable as liquid dosage forms, and a powder suitable for reconstitution with an appropriate solution as solid dosage forms. For inhalation administration, solutions, sprays, dry powders, and aerosols may be acceptable dosage form. For topical (including buccal and sublingual) or transdermal administration, powders, sprays, ointments, pastes, creams, lotions, gels, solutions, and patches may be acceptable dosage form. For vaginal administration, pessaries, tampons, creams, gels, pastes, foams and spray may be acceptable dosage form.

The quantity of active ingredient in a unit dosage form of composition is a therapeutically effective amount and is varied according to the particular treatment involved. As used herein, the term “therapeutically effective amount” refers to an amount of a molecule, compound, or composition comprising the molecule or compound to treat, ameliorate, or prevent an identified disease or condition, or to exhibit a detectable therapeutic or inhibitory effect. The effect can be detected by any assay method known in the art. The precise effective amount for a subject will depend upon the subject's body weight, size, and health; the nature and extent of the condition; the rate of administration; the therapeutic or combination of therapeutics selected for administration; and the discretion of the prescribing physician. Therapeutically effective amounts for a given situation can be determined by routine experimentation that is within the skill and judgment of the clinician.

In some embodiments, the pharmaceutical compositions of the present disclosure may be in a form of formulation for oral administration.

In certain embodiments, the pharmaceutical compositions of the present disclosure may be in the form of tablet formulations. Suitable pharmaceutically-acceptable excipients for a tablet formulation include, for example, inert diluents such as lactose, sodium carbonate, calcium phosphate or calcium carbonate, granulating and disintegrating agents such as corn starch or algenic acid; binding agents such as starch; lubricating agents such as magnesium stearate, stearic acid or talc; preservative agents such as ethyl or propyl p-hydroxybenzoate, and anti-oxidants, such as ascorbic acid. Tablet formulations may be uncoated or coated either to modify their disintegration and the subsequent absorption of the active ingredient within the gastrointestinal tract, or to improve their stability and/or appearance, in either case using conventional coating agents and procedures well known in the art.

In certain embodiments, the pharmaceutical compositions of the present disclosure may be in a form of hard gelatin capsules in which the active ingredient is mixed with an inert solid diluent, for example, calcium carbonate, calcium phosphate or kaolin, or as soft gelatin capsules in which the active ingredient is mixed with water or an oil such as peanut oil, liquid paraffin, or olive oil.

In certain embodiments, the pharmaceutical compositions of the present disclosure may be in the form of aqueous suspensions, which generally contain the active ingredient in finely powdered form together with one or more suspending agents, such as sodium carboxymethylcellulose, methylcellulose, hydroxypropylmethylcellulose, sodium alginate, polyvinyl-pyrrolidone, gum tragacanth and gum acacia; dispersing or wetting agents such as lecithin or condensation products of an alkylene oxide with fatty acids (for example polyoxethylene stearate), or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives (such as ethyl or propyl p-hydroxybenzoate, anti-oxidants (such as ascorbic acid), coloring agents, flavoring agents, and/or sweetening agents (such as sucrose, saccharine or aspartame).

In certain embodiments, the pharmaceutical compositions of the present disclosure may be in the form of oily suspensions, which generally contain suspended active ingredient in a vegetable oil (such as arachis oil, olive oil, sesame oil or coconut oil) or in a mineral oil (such as liquid paraffin). The oily suspensions may also contain a thickening agent such as beeswax, hard paraffin or cetyl alcohol. Sweetening agents such as those set out above, and flavoring agents may be added to provide a palatable oral preparation. These compositions may be preserved by the addition of an anti-oxidant such as ascorbic acid.

In certain embodiments, the pharmaceutical compositions of the present disclosure may be in the form of oil-in-water emulsions. The oily phase may be a vegetable oil, such as olive oil or arachis oil, or a mineral oil, such as for example liquid paraffin or a mixture of any of these. Suitable emulsifying agents may be, for example, naturally-occurring gums such as gum acacia or gum tragacanth, naturally-occurring phosphatides such as soya bean, lecithin, esters or partial esters derived from fatty acids and hexitol anhydrides (for example sorbitan monooleate) and condensation products of the said partial esters with ethylene oxide such as polyoxyethylene sorbitan monooleate. The emulsions may also contain sweetening, flavoring and preservative agents.

In certain embodiments, the pharmaceutical compositions provided herein may be in the form of syrups and elixirs, which may contain sweetening agents such as glycerol, propylene glycol, sorbitol, aspartame or sucrose, a demulcent, a preservative, a flavoring and/or coloring agent.

In some embodiments, the pharmaceutical compositions of the present disclosure may be in a form of formulation for injection administration.

In certain embodiments, the pharmaceutical compositions of the present disclosure may be in the form of a sterile injectable preparation, such as a sterile injectable aqueous or oleaginous suspension. This suspension may be formulated according to the known art using those suitable dispersing or wetting agents and suspending agents, which have been mentioned above. The sterile injectable preparation may also be a sterile injectable solution or suspension in a non-toxic parenterally acceptable diluent or solvent, such as a solution in 1,3-butanediol or prepared as a lyophilized powder. Among the acceptable vehicles and solvents that may be employed are water, Ringer's solution and isotonic sodium chloride solution.

In addition, sterile fixed oils may conventionally be employed as a solvent or suspending medium. For this purpose any bland fixed oil may be employed including synthetic mono- or diglycerides. In addition, fatty acids such as oleic acid may likewise be used in the preparation of injectables.

In some embodiments, the pharmaceutical compositions of the present disclosure may be in a form of formulation for inhalation administration.

In certain embodiments, the pharmaceutical compositions of the present disclosure may be in the form of aqueous and nonaqueous (e.g., in a fluorocarbon propellant) aerosols containing any appropriate solvents and optionally other compounds such as, but not limited to, stabilizers, antimicrobial agents, antioxidants, pH modifiers, surfactants, bioavailability modifiers and combinations of these. The carriers and stabilizers vary with the requirements of the particular compound, but typically include nonionic surfactants (Tweens, Pluronics, or polyethylene glycol), innocuous proteins like serum albumin, sorbitan esters, oleic acid, lecithin, amino acids such as glycine, buffers, salts, sugars or sugar alcohols.

In some embodiments, the pharmaceutical compositions of the present disclosure may be in a form of formulation for topical or transdermal administration.

In certain embodiments, the pharmaceutical compositions provided herein may be in the form of creams, ointments, gels and aqueous or oily solutions or suspensions, which may generally be obtained by formulating an active ingredient with a conventional, topically acceptable excipients such as animal and vegetable fats, oils, waxes, paraffins, starch, tragacanth, cellulose derivatives, polyethylene glycols, silicones, bentonites, silicic acid, talc and zinc oxide, or mixtures thereof.

In certain embodiments, the pharmaceutical compositions provided herein may be formulated in the form of transdermal skin patches that are well known to those of ordinary skill in the art.

Besides those representative dosage forms described above, pharmaceutically acceptable excipients and carriers are generally known to those skilled in the art and are thus included in the present disclosure. Such excipients and carriers are described, for example, in “Remingtons Pharmaceutical Sciences” Mack Pub. Co., New Jersey (1991), in “Remington: The Science and Practice of Pharmacy”, Ed. University of the Sciences in Philadelphia, 21^{st} Edition, LWW (2005), which are incorporated herein by reference.

In some embodiments, the pharmaceutical compositions of the present disclosure can be formulated as a single dosage form. The amount of the compounds provided herein in the single dosage form will vary depending on the subject treated and particular mode of administration.

In some embodiments, the pharmaceutical compositions of the present disclosure can be formulated so that a dosage of between 0.001-1000 mg/kg body weight/day, for example, 0.01-800 mg/kg body weight/day, 0.01-700 mg/kg body weight/day, 0.01-600 mg/kg body weight/day, 0.01-500 mg/kg body weight/day, 0.01-400 mg/kg body weight/day, 0.01-300 mg/kg body weight/day, 0.1-200 mg/kg body weight/day, 0.1-150 mg/kg body weight/day, 0.1-100 mg/kg body weight/day, 0.5-100 mg/kg body weight/day, 0.5-80 mg/kg body weight/day, 0.5-60 mg/kg body weight/day, 0.5-50 mg/kg body weight/day, 1-50 mg/kg body weight/day, 1-45 mg/kg body weight/day, 1-40 mg/kg body weight/day, 1-35 mg/kg body weight/day, 1-30 mg/kg body weight/day, 1-25 mg/kg body weight/day of the compounds provided herein, or a pharmaceutically acceptable salt thereof, can be administered. In some instances, dosage levels below the lower limit of the aforesaid range may be more than adequate, while in other cases still larger doses may be employed without causing any harmful side effect, provided that such larger doses are first divided into several small doses for administration throughout the day. For further information on routes of administration and dosage regimes, see Chapter 25.3 in Volume 5 of Comprehensive Medicinal Chemistry (Corwin Hansch; Chairman of Editorial Board), Pergamon Press 1990, which is specifically incorporated herein by reference.

In some embodiments, the pharmaceutical compositions of the present disclosure can be formulated as short-acting, fast-releasing, long-acting, and sustained-releasing. Accordingly, the pharmaceutical formulations of the present disclosure may also be formulated for controlled release or for slow release.

In a further aspect, there is also provided veterinary compositions comprising one or more molecules or compounds of the present disclosure or pharmaceutically acceptable salts thereof and a veterinary carrier. Veterinary carriers are materials useful for the purpose of administering the composition and may be solid, liquid or gaseous materials which are otherwise inert or acceptable in the veterinary art and are compatible with the active ingredient. These veterinary compositions may be administered parenterally, orally or by any other desired route.

The pharmaceutical compositions or veterinary compositions may be packaged in a variety of ways depending upon the method used for administering the drug. For example, an article for distribution can include a container having deposited therein the compositions in an appropriate form. Suitable containers are well known to those skilled in the art and include materials such as bottles (plastic and glass), sachets, ampoules, plastic bags, metal cylinders, and the like. The container may also include a tamper-proof assemblage to prevent indiscreet access to the contents of the package. In addition, the container has deposited thereon a label that describes the contents of the container. The label may also include appropriate warnings. The compositions may also be packaged in unit-dose or multi-dose containers, for example sealed ampoules and vials, and may be stored in a freeze-dried (lyophilized) condition requiring only the addition of the sterile liquid carrier, for example water, for injection immediately prior to use. Extemporaneous injection solutions and suspensions are prepared from sterile powders, granules and tablets of the kind previously described.

In a further aspect, there is also provided pharmaceutical compositions comprise one or more compounds of the present disclosure, or a pharmaceutically acceptable salt thereof, as a first active ingredient, and a second active ingredient.

In some embodiments, the second active ingredient has complementary activities to the compound provided herein such that they do not adversely affect each other. Such ingredients are suitably present in combination in amounts that are effective for the purpose intended.

In some embodiments, the second active ingredient can include:
(i) antiproliferative/antineoplastic drugs and combinations thereof, as used in medical oncology such as alkylating agents (for example cis-platin, carboplatin, cyclophosphamide, nitrogen mustard, melphalan, chlorambucil, busulphan and nitrosoureas); antimetabolites (for example antifolates such as fluoropyrimidines like 5-fluorouracil and tegafur, raltitrexed, methotrexate, cytosine arabinoside, hydroxyurea and gemcitabine); antitumour antibiotics (for example anthracyclines like adriamycin, bleomycin, doxorubicin, daunomycin, epirubicin, idarubicin, mitomycin-C, dactinomycin and mithramycin); antimitotic agents (for example vinca alkaloids like vincristine, vinblastine, vindesine and vinorelbine and taxoids like paclitaxel and taxotere); and topoisomerase inhibitors (for example epipodophyllotoxins like etoposide and teniposide, amsacrine, topotecan and camptothecins);
(ii) cytostatic agents such as antioestrogens (for example tamoxifen, toremifene, raloxifene, droloxifene and iodoxyfene), oestrogen receptor down regulators (for example fulvestrant), antiandrogens (for example bicalutamide, flutamide, nilutamide and cyproterone acetate), LHRH antagonists or LHRH agonists (for example goserelin, leuprorelin and buserelin), progestogens (for example megestrol acetate), aromatase inhibitors (for example as anastrozole, letrozole, vorazole and exemestane) and inhibitors of 5a-reductase such as finasteride;
(iii) anti-invasion agents (for example c-Src kinase family inhibitors like 4-(6-chloro-2,3-methylenedioxyanilino)-7-[2-(4-methylpiperazin-1-yl)ethoxy]-5-tetrahydropyran-4-yloxyquinazoline (AZD0530) and N-(2-chloro-6-methylphenyl)-2-{6-[4-(2-hydroxyethyl)piperazin-1-yl]-2-methylpyrimidin-4-ylamino}thiazole-5-carboxamide (dasatinib, BMS-354825), and metalloproteinase inhibitors like marimastat and inhibitors of urokinase plasminogen activator receptor function);
(iv) inhibitors of growth factor function: for example such inhibitors include growth factor antibodies and growth factor receptor antibodies (for example the anti-erbB2 antibody trastuzumab [Herceptin™] and the anti-erbB1 antibody cetuximab [C225]); such inhibitors also include, for example, tyrosine kinase inhibitors, for example inhibitors of the epidermal growth factor family (for example EGFR family tyrosine kinase inhibitors such as N-(3-chloro-4-fluorophenyl)-7-methoxy-6-(3-morpholinopropoxy)quinazolin-4-amine(gefitinib, ZD 1839), N-(3-ethynylphenyl)-6,7-bis(2-methoxyethoxy)quinazolin-4-amine (erlotinib, OSI-774) and 6-acrylamido-N-(3-chloro-4-fluorophenyl)-7-(3-morpholinopropoxy)quinazolin-4-amine (CI 1033) and erbB2 tyrosine kinase inhibitors such as lapatinib), inhibitors of the hepatocyte growth factor family, inhibitors of the platelet-derived growth factor family such as imatinib, inhibitors of serine/threonine kinases (for example Ras/Raf signalling inhibitors such as farnesyl transferase inhibitors, for example sorafenib (BAY 43-9006)) and inhibitors of cell signalling through MEK and/or Akt kinases;
(v) antiangiogenic agents such as those which inhibit the effects of vascular endothelial growth factor, [for example the anti-vascular endothelial cell growth factor antibody bevacizumab (Avastin™) and VEGF receptor tyrosine kinase inhibitors such as 4-(4-bromo-2-fluoroanilino)-6-methoxy-7-(1-methylpiperidin-4-ylmethoxy)quinazoline (ZD6474; Example 2 within WO 01/32651), 4-(4-fluoro-2-methylindol-5-yloxy)-6-methoxy-7-(3-pyrrolidin-1-ylpropoxy)quinazoline (AZD2171; Example 240 within WO 00/47212), vatalanib (PTK787; WO 98/35985) and SU11248 (sunitinib; WO 01/60814), and compounds that work by other mechanisms (for example linomide, inhibitors of integrin αvβ3 function and angiostatin)];
(vi) vascular damaging agents such as combretastatin A4 and compounds disclosed in International Patent Applications WO 99/02166, WO 00/40529, WO 00/41669, WO 01/92224, WO 02/04434 and WO 02/08213;
(vii) antisense therapies, such as ISIS 2503, an anti-ras antisense agent; (viii) gene therapy approaches, including approaches to replace aberrant genes such as aberrant p53 or aberrant BRCA1 or BRCA2, GDEPT (gene-directed enzyme pro-drug therapy) approaches such as those using cytosine deaminase, thymidine kinase or a bacterial nitroreductase enzyme and approaches to increase patient tolerance to chemotherapy or radiotherapy such as multi-drug resistance gene therapy; and (ix) immunotherapeutic approaches, including ex-vivo and in-vivo approaches to increase the immunogenicity of patient tumour cells, such as transfection with cytokines such as interleukin 2, interleukin 4 or granulocyte-macrophage colony stimulating factor, approaches to decrease T-cell anergy, approaches using transfected immune cells such as cytokine-transfected dendritic cells, approaches using cytokine-transfected tumour cell lines and approaches using anti-idiotypic antibodies.

### Method of Treatment of Disease

In an aspect, the present disclosure provides compounds of Formula (I) or pharmaceutically acceptable salts thereof, which are capable of inhibiting ATR kinase. The inhibitory properties of compounds of Formula (I) may be demonstrated using the test procedures set out herein.

Accordingly, the compounds of Formula (I) may be used in the treatment (therapeutic or prophylactic) of conditions or diseases in a subject which are mediated by ATR kinase.

As used herein, a “subject” refers to a human and a non-human animal. Examples of a non-human animal include all vertebrates, e.g., mammals, such as non-human primates (particularly higher primates), dog, rodent (e.g., mouse or rat), guinea pig, cat, and non-mammals, such as birds, amphibians, reptiles, etc. In a preferred embodiment, the subject is a human. In another embodiment, the subject is an experimental animal or animal suitable as a disease model.

In some embodiments, the compounds of Formula (I) can be used as antitumour agents. In some embodiments, the compounds of Formula (I) can be used as anti-proliferative, apoptotic and/or anti-invasive agents in the containment and/or treatment of solid and/or liquid tumour disease. In certain embodiments, the compounds of Formula (I) are useful in the prevention or treatment of those tumours which are sensitive to inhibition of ATR. In certain embodiments, the compounds of Formula (I) are useful in the prevention or treatment of those tumours which are mediated alone or in part by ATR.

In some embodiments, the compounds of Formula (I) are useful for the treatment of proliferative diseases, including malignant diseases such as cancer as well as non-malignant diseases such as inflammatory diseases, obstructive airways diseases, immune diseases or cardiovascular diseases.

In some embodiments, the compounds of Formula (I) are useful for the treatment of cancer, for example but not limited to, haematologic malignancies such as leukaemia, multiple myeloma, lymphomas such as Hodgkin's disease, non-Hodgkin's lymphomas (including mantle cell lymphoma), and myelodysplastic syndromes, and also solid tumours and their metastases such as breast cancer, lung cancer (non-small cell lung cancer (NSCL), small cell lung cancer (SCLC), squamous cell carcinoma), endometrial cancer, tumours of the central nervous system such as gliomas, dysembryoplastic neuroepithelial tumour, glioblastoma multiforme, mixed gliomas, medulloblastoma, retinoblastoma, neuroblastoma, germinoma and teratoma, cancers of the gastrointestinal tract such as gastric cancer, oesophagal cancer, hepatocellular (liver) carcinoma, cholangiocarcinomas, colon and rectal carcinomas, cancers of the small intestine, pancreatic cancers, cancers of the skin such as melanomas (in particular metastatic melanoma), thyroid cancers, cancers of the head and neck and cancers of the salivary glands, prostate, testis, ovary, cervix, uterus, vulva, bladder, kidney (including renal cell carcinoma, clear cell and renal oncocytoma), squamous cell carcinomas, sarcomas such as osteosarcoma, chondrosarcoma, leiomyosarcoma, soft tissue sarcoma, Ewing's sarcoma, gastrointestinal stromal tumour (GIST), Kaposi's sarcoma, and paediatric cancers such as rhabdomyosarcomas and neuroblastomas.

In some embodiments, the compounds of Formula (I) are useful for the treatment of autoimmune and/or inflammatory diseases, for example but not limited to, allergy, Alzheimer's disease, acute disseminated encephalomyelitis, Addison's disease, ankylosing spondylitis, antiphospholipid antibody syndrome, asthma, atherosclerosis, autoimmune hemolytic anemia, autoimmune hemolytic and thrombocytopenic states, autoimmune hepatitis, autoimmune inner ear disease, bullous pemphigoid, coeliac disease, chagas disease, chronic obstructive pulmonary disease, chronic Idiopathic thrombocytopenic purpura (ITP), churg-strauss syndrome, Crohn's disease, dermatomyositis, diabetes mellitus type 1, endometriosis, Goodpasture's syndrome (and associated glomerulonephritis and pulmonary hemorrhage), graves' disease, guillain-barre syndrome, hashimoto's disease, hidradenitis suppurativa, idiopathic thrombocytopenic purpura, interstitial cystitis, irritable bowel syndrome, lupus erythematosus, morphea, multiple sclerosis, myasthenia gravis, narcolepsy, neuromyotonia, Parkinson's disease, pemphigus vulgaris, pernicious anaemia, polymyositis, primary biliary cirrhosis, psoriasis, psoriatic arthritis, rheumatoid arthritis, schizophrenia, septic shock, scleroderma, Sjogren's disease, systemic lupus erythematosus (and associated glomerulonephritis), temporal arteritis, tissue graft rejection and hyperacute rejection of transplanted organs, vasculitis (ANCA-associated and other vasculitides), vitiligo, and Wegener's granulomatosis.

As used herein, the term “therapy” is intended to have its normal meaning of dealing with a disease in order to entirely or partially relieve one, some or all of its symptoms, or to correct or compensate for the underlying pathology, thereby achieving beneficial or desired clinical results. For purposes of this disclosure, beneficial or desired clinical results include, but are not limited to, alleviation of symptoms, diminishment of extent of disease, stabilized (i.e., not worsening) state of disease, delay or slowing of disease progression, amelioration or palliation of the disease state, and remission (whether partial or total), whether detectable or undetectable. “Therapy” can also mean prolonging survival as compared to expected survival if not receiving it. Those in need of therapy include those already with the condition or disorder as well as those prone to have the condition or disorder or those in which the condition or disorder is to be prevented. The term “therapy” also encompasses prophylaxis unless there are specific indications to the contrary. The terms “therapeutic” and “therapeutically” should be interpreted in a corresponding manner.

As used herein, the term “prophylaxis” or “prophylactic” is intended to have its normal meaning and includes primary prophylaxis to prevent the development of the disease and secondary prophylaxis whereby the disease has already developed and the patient is temporarily or permanently protected against exacerbation or worsening of the disease or the development of new symptoms associated with the disease.

The term “treatment” is used synonymously with “therapy”. Similarly the term “treat” can be regarded as “applying therapy” where “therapy” is as defined herein.

In a further aspect, the present disclosure provides use of the compound of the present disclosure or a pharmaceutically acceptable salt thereof or the pharmaceutical composition of the present disclosure for use in therapy, for example, for use in therapy associated with ATR kinase.

In a further aspect, the present disclosure provides use of the compound of the present disclosure or a pharmaceutically acceptable salt thereof or the pharmaceutical composition of the present disclosure, in the manufacture of a medicament for treating cancer.

In a further aspect, the present disclosure provides use of the compound of the present disclosure or a pharmaceutically acceptable salt thereof or the pharmaceutical composition of the present disclosure, in the manufacture of a medicament for treating cancer.

In another aspect, the present disclosure provides a compound of the present disclosure or a pharmaceutically acceptable salt thereof or a pharmaceutical composition of the present disclosure, for use in the treatment of cancer.

In some embodiments, the compounds of Formula (I) can be used further combination with other biologically active ingredients (such as, but not limited to, a second and different antineoplastic agent) and non-drug therapies (such as, but not limited to, surgery or radiation treatment). For instance, the compounds of Formula (I) can be used in combination with other pharmaceutically active compounds, or non-drug therapies, preferably compounds that are able to enhance the effect of the compounds of Formula (I). The compounds of Formula (I) can be administered simultaneously (as a single preparation or separate preparation) or sequentially to the other therapies. In general, a combination therapy envisions administration of two or more drugs/treatments during a single cycle or course of therapy.

In some embodiments, the compounds of Formula (I) are used in combination with one or more of traditional chemotherapeutic agents, which encompass a wide range of therapeutic treatments in the field of oncology. These agents are administered at various stages of the disease for the purposes of shrinking tumors, destroying remaining cancer cells left over after surgery, inducing remission, maintaining remission and/or alleviating symptoms relating to the cancer or its treatment.

In some embodiments, the compounds of Formula (I) are used in combination with one or more targeted anti-cancer agents that modulate protein kinases involved in various disease states.

In some embodiments, the compounds of Formula (I) are used in combination with one or more targeted anti-cancer agents that modulate non-kinase biological targets, pathway, or processes.

In some embodiments, the compounds of Formula (I) are used in combination with one or more of other anti-cancer agents that include, but are not limited to, gene therapy, RNAi cancer therapy, chemoprotective agents (e.g., amfostine, mesna, and dexrazoxane), drug-antibody conjugate (e.g. brentuximab vedotin, ibritumomab tioxetan), cancer immunotherapy such as Interleukin-2, cancer vaccines (e.g., sipuleucel-T) or monoclonal antibodies (e.g., Bevacizumab, Alemtuzumab, Rituximab, Trastuzumab, etc.).

In some embodiments, the compounds of Formula (I) are used in combination with one or more anti-inflammatory agent including but not limited to NSAIDs, non-specific and COX-2 specific cyclooxgenase enzyme inhibitors, gold compounds, corticosteroids, methotrexate, tumor necrosis factor receptor (TNF) receptors antagonists, immunosuppressants and methotrexate.

In some embodiments, the compounds of Formula (I) are used in combination with radiation therapy or surgeries. Radiation is commonly delivered internally (implantation of radioactive material near cancer site) or externally from a machine that employs photon (x-ray or gamma-ray) or particle radiation. Where the combination therapy further comprises radiation treatment, the radiation treatment may be conducted at any suitable time so long as a beneficial effect from the co-action of the combination of the therapeutic agents and radiation treatment is achieved.

Accordingly, in a further aspect, the present disclosure provides a method for treating diseases associated with ATR kinase in a subject in need thereof, comprising administering an effective amount of a compound of the present disclosure or a pharmaceutically acceptable salt thereof or the pharmaceutical composition of the present disclosure to the subject.

### EXAMPLES

For the purpose of illustration, the following examples are included. However, it is to be understood that these examples do not limit the present disclosure and are only meant to suggest a method of practicing the present disclosure. Persons skilled in the art will recognize that the chemical reactions described may be readily adapted to prepare a number of other compounds of the present disclosure, and alternative methods for preparing the compounds of the present disclosure are deemed to be within the scope of the present disclosure. For example, the synthesis of non-exemplified compounds according to the present disclosure may be successfully performed by modifications apparent to those skilled in the art, e.g., by appropriately protecting interfering groups, by utilizing other suitable reagents and building blocks known in the art other than those described, and/or by making routine modifications of reaction conditions. Alternatively, other reactions disclosed herein or known in the art will be recognized as having applicability for preparing other compounds of the present disclosure.

### Example 1

### Synthesis of (R)-3-methyl-4-(7-(1-methyl-1H-pyrazol-5-yl)-3-(1H-pyrazol-5-yl)pyrazolo[1,5-a]pyrimidin-5-yl)morpholine

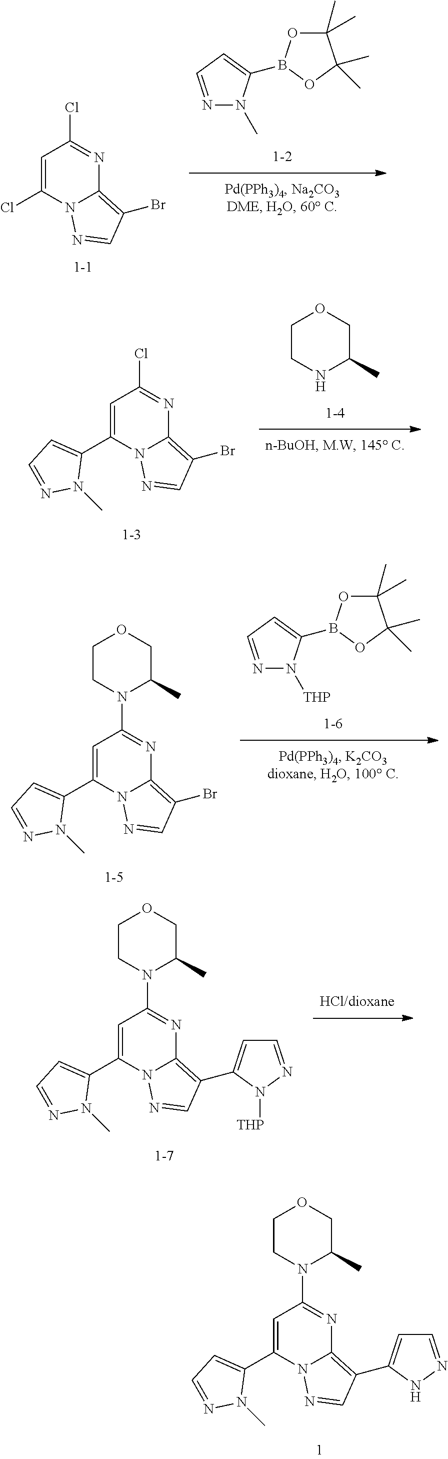

### Step 1. 3-bromo-5-chloro-7-(1-methyl-1H-pyrazol-5-yl)pyrazolo[1,5-a]pyrimidine

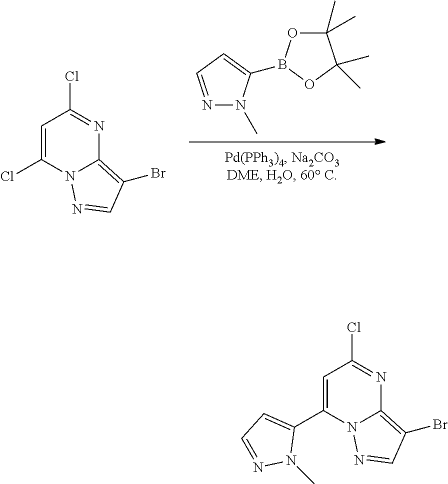 [SMILES: Clc1cc(Cl)n2ncc(Br)c2n1 ; Cn1nccc1B1OC(C)(C)C(C)(C)O1 ; Cn1nccc1-c1cc(Cl)nc2c(Br)cnn12]

A mixture of 3-bromo-5,7-dichloropyrazolo[1,5-a]pyrimidine (1.0 g, 3.74 mmol), 1-methyl-5-(tetramethyl-1,3,2-dioxaborolan-2-yl)-1H-pyrazole (0.78 g, 3.74 mmol), Pd(PPh₃)₄ (0.22 g, 0.18 mmol) and Na₂CO₃ (0.79 g, 7.49 mmol) in co-solvent of DME (60 mL) and H₂O (12 mL) was stirred at 60° C. for 4 h. LC-MS showed the reaction was complete. The reaction mixture was diluted with H₂O (50 mL), then extracted with EA (60 mL×3). The combined organic layer was washed with brine, dried over anhydrous Na₂SO₄, filtered and concentrated. The residue was purified by column chromatography on silica gel (PE:EA=2:1, V/V) to give the desired product (187 mg, yield: 16%). LC/MS (ESI): m/z 312 [M+H]⁺.

### Step 2. (R)-4-(3-bromo-7-(1-methyl-1H-pyrazol-5-yl)pyrazolo[1,5-a]pyrimidin-5-yl)-3-methylmorpholine

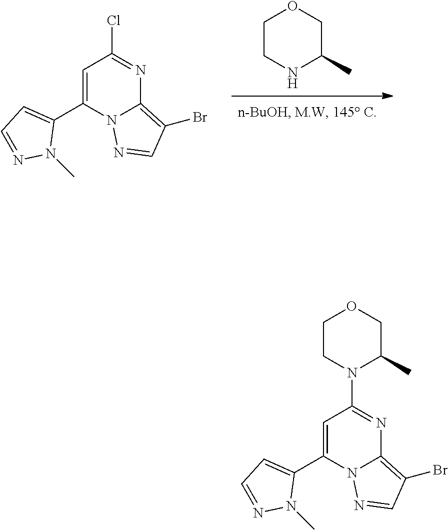

A mixture of 5-{3-bromo-5-chloropyrazolo[1,5-a]pyrimidin-7-yl}-1-methyl-1H-pyrazole (167 mg, 0.53 mmol) and (3R)-3-methylmorpholine (486 mg, 4.80 mmol) in n-BuOH (2 mL) was stirred at 145° C. for 1 h under microwave irradiation. LC-MS showed the reaction was complete. The reaction mixture was diluted with H₂O (20 mL) and extracted with EA (30 mL×3). The combined organic layer was washed with brine, dried over anhydrous Na₂SO₄, filtered and concentrated. The residue was purified by column chromatography on silica gel (PE:EA=1:1, V/V) to give the desired product (148 mg, yield: 73%). LC/MS (ESI): m/z 377 [M+H]⁺.

### Step 3. (3R)-3-methyl-4-(7-(1-methyl-1H-pyrazol-5-yl)-3-(1-(tetrahydro-2H-pyran-2-yl)-1H-pyrazol-5-yl)pyrazolo[1,5-a]pyrimidin-5-yl)morpholine

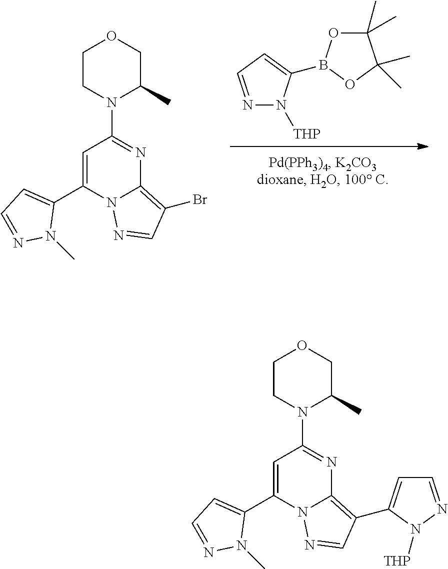

A mixture of (3R)-4-[3-bromo-7-(1-methyl-1H-pyrazol-5-yl)pyrazolo[1,5-a]pyrimidin-5-yl]-3-methylmorpholine (128 mg, 0.33 mmol), 1-(oxan-2-yl)-5-(tetramethyl-1,3,2-dioxaborolan-2-yl)-1H-pyrazole (188 mg, 0.67 mmol), Pd(PPh₃)₄ (39 mg, 0.03 mmol) and K₂CO₃ (117 mg, 0.84 mmol) in co-solvent of dioxane (5 mL) and H₂O (1 mL) was stirred at 100° C. for 16 h under N₂ atmosphere. LC-MS showed the reaction was complete. The reaction mixture was diluted with H₂O (20 mL), then extracted with EA (30 mL×3). The combined organic layer was washed with brine, dried over anhydrous Na₂SO₄, filtered and concentrated. The residue was purified by column chromatography on silica gel (PE:EA=1:2, V/V) to give the desired product (59 mg, yield: 38%). LC/MS (ESI): m/z 449 [M+H]⁺.

### Step 4. (R)-3-methyl-4-(7-(1-methyl-1H-pyrazol-5-yl)-3-(1H-pyrazol-5-yl)pyrazolo [1,5-a]pyrimidin-5-yl)morpholine

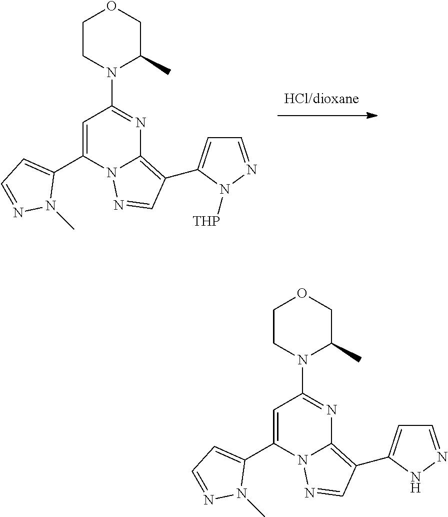

A mixture of (3R)-3-methyl-4-[7-(1-methyl-1H-pyrazol-5-yl)-3-[1-(oxan-2-yl)-1H-pyrazol-5-yl]pyrazolo[1,5-a]pyrimidin-5-yl]morpholine (59 mg, 0.13 mmol) in HCl solution (4 M in dioxane, 3 mL) was stirred at room temperature for 0.5 h. LC-MS showed the reaction was complete. The reaction mixture was concentrated under vacuo. The residue was purified by Prep-HPLC (C₁₈, 10-95%, MeOH in H₂O with 0.1% HCOOH) to give the desired product (44.2 mg, yield: 92%). LC/MS (ESI): m/z 365 [M+H]⁺. ¹H NMR (400 MHz, DMSO) δ 12.67 (s, 1H), 8.42 (s, 1H), 8.30 (s, 1H), 7.63 (d, J=1.9 Hz, 2H), 6.90 (s, 1H), 6.80 (d, J=1.9 Hz, 1H), 6.76 (s, 1H), 4.59 (s, 1H), 4.26 (d, J=13.5 Hz, 1H), 4.00 (dd, J=11.5, 3.3 Hz, 1H), 3.85 (s, 3H), 3.78 (d, J=11.4 Hz, 1H), 3.67 (dd, J=11.5, 2.9 Hz, 1H), 3.55-3.49 (m, 1H), 3.27-3.24 (m, 1H), 1.29 (d, J=6.7 Hz, 3H).

### Example 2

### Synthesis of (R)-3-methyl-4-(7-(1-methyl-H-pyrazol-5-yl)-3-(1H-pyrazol-4-yl)pyrazolo[1,5-a]pyrimidin-5-yl)morpholine

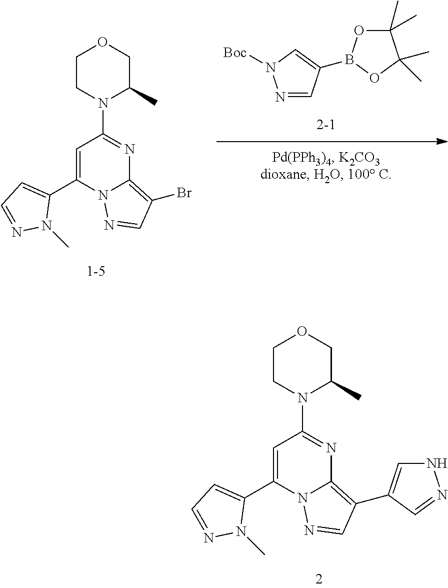 [SMILES: C[C@@H]1COCCN1c1cc(-c2ccnn2C)n2ncc(Br)c2n1 ; CC(C)(C)OC(=O)n1cc(B2OC(C)(C)C(C)(C)O2)cn1 ; C[C@@H]1COCCN1c1cc(-c2ccnn2C)n2ncc(-c3cn[nH]c3)c2n1]

### Step 1. (R)-3-methyl-4-(7-(1-methyl-1H-pyrazol-5-yl)-3-(1H-pyrazol-4-yl)pyrazolo [1,5-a]pyrimidin-5-yl)morpholine

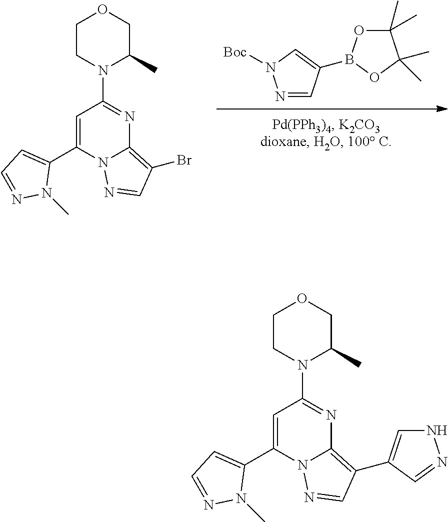 [SMILES: C[C@@H]1COCCN1c1cc(-c2ccnn2C)n2ncc(Br)c2n1 ; CC(C)(C)OC(=O)n1cc(B2OC(C)(C)C(C)(C)O2)cn1 ; C[C@@H]1COCCN1c1cc(-c2ccnn2C)n2ncc(-c3cn[nH]c3)c2n1]

A mixture of (3R)-4-[3-bromo-7-(1-methyl-1H-pyrazol-5-yl)pyrazolo[1,5-a]pyrimidin-5-yl]-3-methylmorpholine (100 mg, 0.26 mmol), tert-butyl 4-(tetramethyl-1,3,2-dioxaborolan-2-yl)-1H-pyrazole-1-carboxylate (155 mg, 0.53 mmol), Pd(PPh₃)₄ (30 mg, 0.02 mmol) and K₂CO₃ (91 mg, 0.66 mmol) in co-solvent of dioxane (3 mL) and H₂O (0.6 mL) was stirred at 100° C. for 16 h under N₂ atmosphere. LC-MS showed the reaction was complete. The reaction mixture was diluted with H₂O (20 mL), then extracted with EA (30 mL×3). The combined organic layer was washed with brine, dried over anhydrous Na₂SO₄, filtered and concentrated. The residue was purified by Prep-HPLC (C₁₈, 10-95%, MeOH in H₂O with 0.1% HCOOH) to give the desired product (34.5 mg, yield: 36%). LC/MS (ESI): m/z 365 [M+H]⁺. ¹H NMR (400 MHz, DMSO) δ 12.78 (s, 1H), 8.22 (s, 1H), 8.05 (s, 2H), 7.62 (d, J=1.9 Hz, 1H), 6.85 (s, 1H), 6.79 (d, J=1.9 Hz, 1H), 4.60-4.53 (m, 1H), 4.21 (d, J=12.3 Hz, 1H), 4.00 (dd, J=11.1, 3.1 Hz, 1H), 3.84 (s, 3H), 3.79-3.77 (m, 1H), 3.67 (dd, J=11.5, 3.0 Hz, 1H), 3.55-3.49 (m, 1H), 3.27-3.23 (m, 1H), 1.28 (d, J=6.7 Hz, 3H).

### Example 3

### Synthesis of (R)-3-methyl-4-(7-(1-methyl-1H-pyrazol-5-yl)-3-(pyridin-3-yl)pyrazolo[1,5-a]pyrimidin-5-yl)morpholine

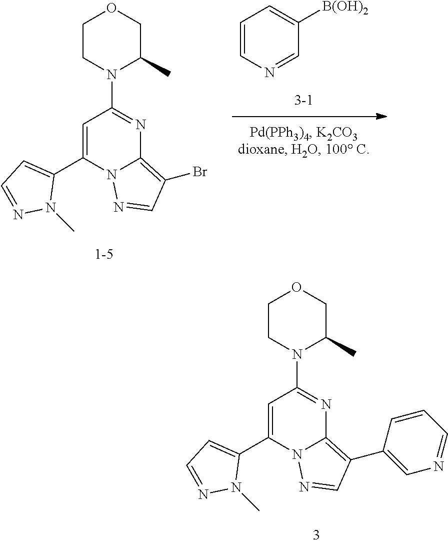 [SMILES: C[C@@H]1COCCN1c1cc(-c2ccnn2C)n2ncc(Br)c2n1 ; OB(O)c1cccnc1 ; C[C@@H]1COCCN1c1cc(-c2ccnn2C)n2ncc(-c3cccnc3)c2n1]

### Step 1. (R)-3-methyl-4-(7-(1-methyl-1H-pyrazol-5-yl)-3-(pyridin-3-yl)pyrazolo[1,5-a]pyrimidin-5-yl)morpholine [1,5-a]pyrimidine

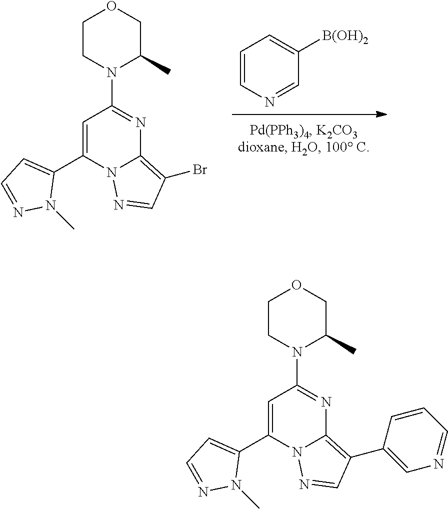 [SMILES: C[C@@H]1COCCN1c1cc(-c2ccnn2C)n2ncc(Br)c2n1 ; OB(O)c1cccnc1 ; C[C@@H]1COCCN1c1cc(-c2ccnn2C)n2ncc(-c3cccnc3)c2n1]

A mixture of (3R)-4-[3-bromo-7-(1-methyl-1H-pyrazol-5-yl)pyrazolo[1,5-a]pyrimidin-5-yl]-3-methylmorpholine (100 mg, 0.26 mmol), pyridin-3-ylboronic acid (65.2 mg, 0.53 mmol), Pd(PPh₃)₄ (30 mg, 0.02 mmol) and K₂CO₃ (91 mg, 0.66 mmol) in co-solvent of dioxane (2 mL) and H₂O (0.4 mL) was stirred at 100° C. for 16 h under N₂ atmosphere. LC-MS showed the reaction was complete. The reaction mixture was diluted with H₂O (20 mL), then extracted with DCM (30 mL×3). The combined organic layer was washed with brine, dried over anhydrous Na₂SO₄, filtered and concentrated. The residue was purified by Prep-HPLC (C₁₈, 10-95%, MeOH in H₂O with 0.1% HCOOH) to give the desired product (34.0 mg, yield: 34%). LC/MS (ESI): m/z 376 [M+H]⁺. ¹H NMR (400 MHz, DMSO) δ 9.31 (d, J=1.9 Hz, 1H), 8.58 (s, 1H), 8.42 (dt, J=8.0, 1.8 Hz, 1H), 8.36 (dd, J=4.7, 1.5 Hz, 1H), 8.15 (s, 0.5H), 7.65 (d, J=1.9 Hz, 1H), 7.43-7.40 (m, 1H), 6.96 (s, 1H), 6.82 (d, J=1.9 Hz, 1H), 4.59-4.58 (m, 1H), 4.25 (d, J=13.2 Hz, 1H), 4.02 (dd, J=11.4, 3.4 Hz, 1H), 3.87 (s, 3H), 3.80 (d, J=11.4 Hz, 1H), 3.68 (dd, J=11.4, 2.9 Hz, 1H), 3.59-3.50 (m, 2H), 1.31 (d, J=6.7 Hz, 3H).

### Example 4

### Synthesis of (R)-3-methyl-4-(7-(1-methyl-1H-pyrazol-5-yl)-3-(1H-pyrrol-2-yl)pyrazolo[1,5-a]pyrimidin-5-yl)morpholine

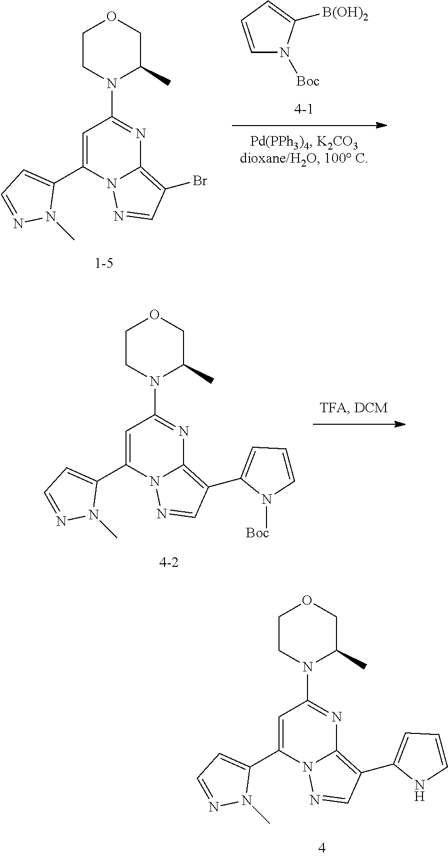 [SMILES: C[C@@H]1COCCN1c1cc(-c2ccnn2C)n2ncc(Br)c2n1 ; CC(C)(C)OC(=O)n1cccc1B(O)O ; C[C@@H]1COCCN1c1cc(-c2ccnn2C)n2ncc(-c3cccn3C(=O)OC(C)(C)C)c2n1 ; C[C@@H]1COCCN1c1cc(-c2ccnn2C)n2ncc(-c3ccc[nH]3)c2n1]

### Step 1. tert-butyl (R)-2-(7-(1-methyl-1H-pyrazol-5-yl)-5-(3-methylmorpholino) pyrazolo[1,5-a]pyrimidin-3-yl)-1H-pyrrole-1-carboxylate

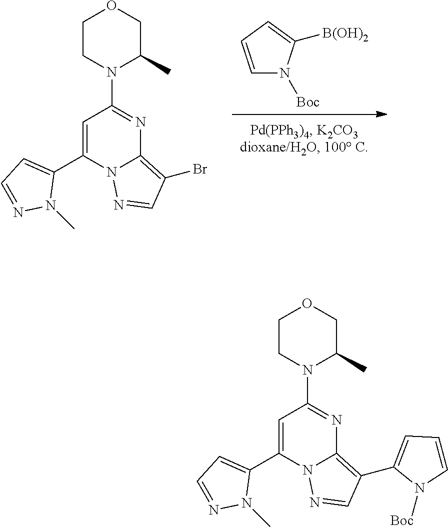 [SMILES: C[C@@H]1COCCN1c1cc(-c2ccnn2C)n2ncc(Br)c2n1 ; CC(C)(C)OC(=O)n1cccc1B(O)O ; C[C@@H]1COCCN1c1cc(-c2ccnn2C)n2ncc(-c3cccn3C(=O)OC(C)(C)C)c2n1]

A mixture of (3R)-4-[3-bromo-7-(1-methyl-1H-pyrazol-5-yl)pyrazolo[1,5-a]pyrimidin-5-yl]-3-methylmorpholine (120 mg, 0.31 mmol), (1-(tert-butoxycarbonyl)-1H-pyrrol-2-yl)boronic acid (134 mg, 0.64 mmol), Pd(PPh₃)₄ (36 mg, 0.03 mmol) and K₂CO₃ (109 mg, 0.79 mmol) in co-solvent of dioxane (4 mL) and H₂O (0.8 mL) was stirred at 100° C. for 16 h under N₂ atmosphere. LC-MS showed the reaction was complete. The reaction mixture was diluted with H₂O (20 mL), then extracted with EA (20 mL×3). The combined organic layer was washed with brine, dried over anhydrous Na₂SO₄, filtered and concentrated. The residue was purified by column chromatography on silica gel (PE:EA=1:2, V/V) to give the desired product (79 mg, yield: 53%). LC/MS (ESI): m/z 464 [M+H]⁺.

### Step 2. (R)-3-methyl-4-(7-(1-methyl-1H-pyrazol-5-yl)-3-(1H-pyrrol-2-yl)pyrazolo [1,5-a]pyrimidin-5-yl)morpholine

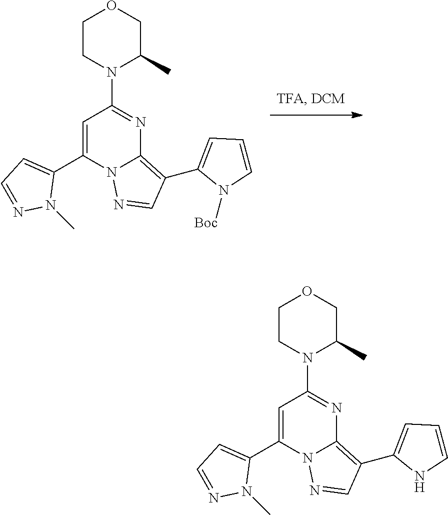 [SMILES: C[C@@H]1COCCN1c1cc(-c2ccnn2C)n2ncc(-c3cccn3C(=O)OC(C)(C)C)c2n1 ; C[C@@H]1COCCN1c1cc(-c2ccnn2C)n2ncc(-c3ccc[nH]3)c2n1]

To a solution of tert-butyl 2-[7-(1-methyl-1H-pyrazol-5-yl)-5-[(3R)-3-methyl morpholin-4-yl]pyrazolo[1,5-a]pyrimidin-3-yl]-1H-pyrrole-1-carboxylate (40 mg, 0.08 mmol) in DCM (3 mL) was added TFA (0.6 mL). The mixture was stirred at room temperature for 2 h. LC-MS showed the reaction was complete. The reaction mixture was concentrated under vacuo. The residue was purified by Prep-HPLC (C₁₈, 10-95%, MeOH in H₂O with 0.1% HCOOH) to give the desired product (13.2 mg, yield: 42%). LC/MS (ESI): m/z 364 [M+H]⁺. ¹H NMR (400 MHz, DMSO) δ 10.80 (s, 1H), 8.25 (s, 1H), 7.63 (d, J=1.9 Hz, 1H), 6.92-6.71 (m, 3H), 6.52 (t, J=3.5 Hz, 1H), 6.09 (dd, J=5.6, 2.6 Hz, 1H), 4.59 (d, J=5.0 Hz, 1H), 4.26 (d, J=13.2 Hz, 1H), 4.01 (dd, J=11.2, 3.1 Hz, 1H), 3.85 (s, 3H), 3.78 (d, J=11.4 Hz, 1H), 3.67 (dd, J=11.5, 2.8 Hz, 1H), 3.52 (td, J=11.9, 2.8 Hz, 1H), 3.30-3.21 (m, 1H), 1.28 (d, J=6.7 Hz, 3H).

### Example 5

### Synthesis of (R)-3-methyl-4-(7-(1-(methylsulfonyl)cyclopropyl)-3-(1H-pyrazol-5-yl)pyrazolo[1,5-a]pyrimidin-5-yl)morpholine

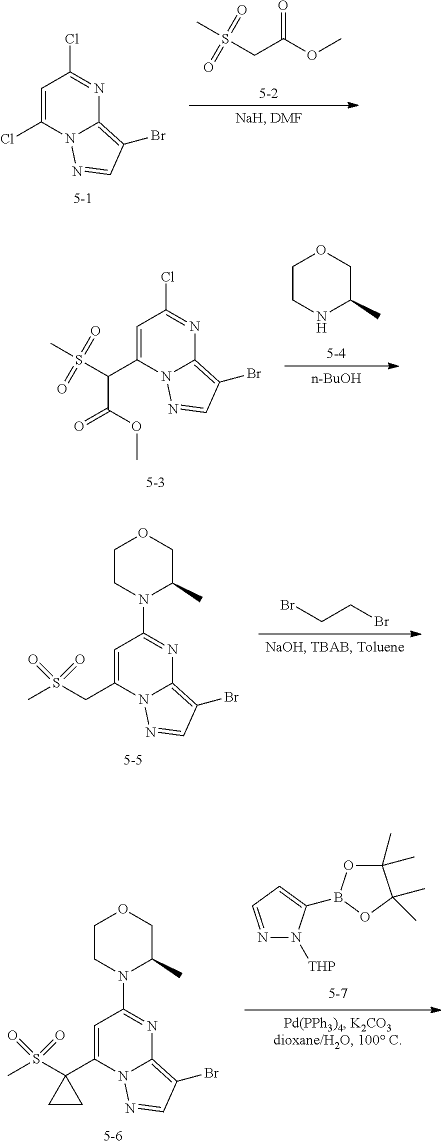

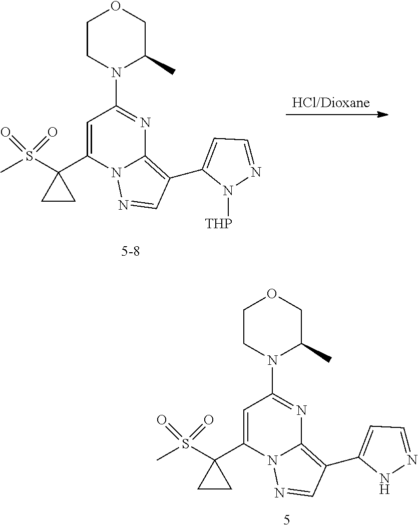

### Step 1. methyl 2-(3-bromo-5-chloropyrazolo[1,5-a]pyrimidin-7-yl)-2-(methylsulfonyl) acetate

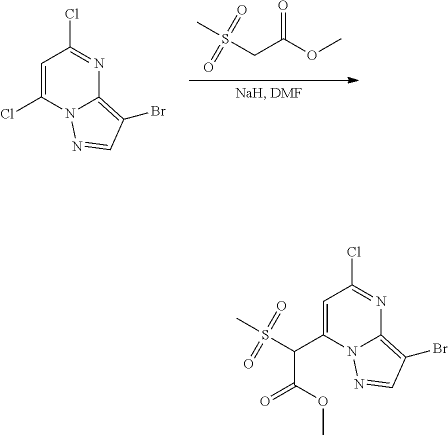

To a solution of methyl 2-methanesulfonylacetate (0.60 g, 3.93 mmol) in DMF (20 mL) at 0° C. was added NaH (0.22 g, 5.62 mmol) portion wise. The mixture was stirred at 0° C. for 30 min, then a solution of 3-bromo-5,7-dichloropyrazolo [1,5-a]pyrimidine (1 g, 3.75 mmol) in DMF (2 mL) was added drop wise. The resulting mixture was stirred at room temperature for 1 h. LC-MS showed the reaction was complete. The reaction mixture was quenched with saturated NH₄Cl aqueous solution and extracted with EA (30 mL×2). The combined organic layer was washed with brine, dried over anhydrous Na₂SO₄, filtered and concentrated. The residue was purified by column chromatography on silica gel (PE:EA=3:1, V/V) to give the desired product (1 g, yield: 69%). LC/MS (ESI) m/z: 382/384 [M+H]⁺. ¹H NMR (400 MHz, DMSO) δ 8.55 (s, 1H), 7.48 (s, 1H), 6.78 (s, 1H), 3.78 (s, 3H), 3.41 (s, 4H).

### Step 2. (R)-4-(3-bromo-7-((methylsulfonyl)methyl)pyrazolo[1,5-a]pyrimidin-5-yl)-3-methylmorpholine

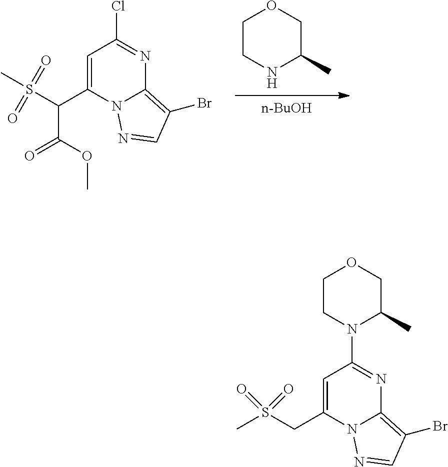

To a solution of methyl 2-(3-bromo-5-chloropyrazolo[1,5-a]pyrimidin-7-yl)-2-(methylsulfonyl)acetate (500 mg, 1.31 mmol) in n-BuOH (15 mL) was added (3R)-3-methylmorpholine (1.19 g, 11.76 mmol). The mixture was stirred at 145° C. for 1 h under microwave irradiation. The reaction mixture was diluted with EA (50 mL), then washed with water and brine, dried over anhydrous Na₂SO₄, filtered and concentrated. The residue was purified by column chromatography on silica gel (PE:EA=2:1, V/V) to give the desired product (280 mg, yield: 77%). LC/MS (ESI) m/z: 389/391 [M+H]⁺.

### Step 3. (R)-4-(3-bromo-7-(1-(methylsulfonyl)cyclopropyl)pyrazolo[1,5-a]pyrimidin-5-yl)-3-methylmorpholine

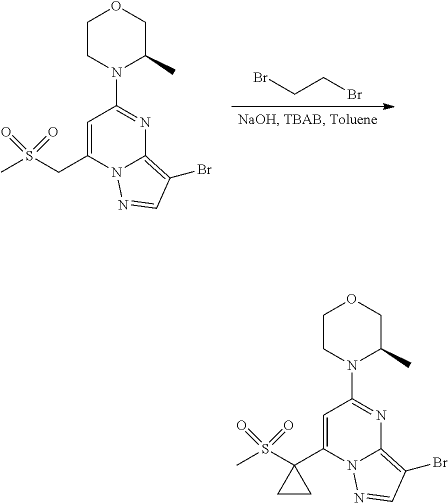

To a solution of (R)-4-(3-bromo-7-((methylsulfonyl)methyl)pyrazolo[1,5-a]pyrimidin-5-yl)-3-methylmorpholine (200 mg, 0.51 mmol) in Toluene (10 mL) were added 1,2-dibromoethane (0.11 mL, 1.28 mmol), NaOH (10 M in H₂O, 0.51 mL, 5.14 mmol) and TBAB (32 mg, 0.10 mmol) successively. The mixture was stirred at 60° C. for 3 h. LC-MS showed the reaction was complete. The reaction mixture was diluted with DCM (40 mL), then washed with water and brine, dried over anhydrous Na₂SO₄, filtered and concentrated. The residue was purified by column chromatography on silica gel (PE:EA=2:1, V/V) to give the desired product (170 mg, yield: 79%). LC/MS (ESI) m/z: 415/417 [M+H]⁺.

### Step 4. (3R)-3-methyl-4-(7-(1-(methylsulfonyl)cyclopropyl)-3-(1-(tetrahydro-2H-pyran-2-yl)-1H-pyrazol-5-yl)pyrazolo[1,5-a]pyrimidin-5-yl)morpholine

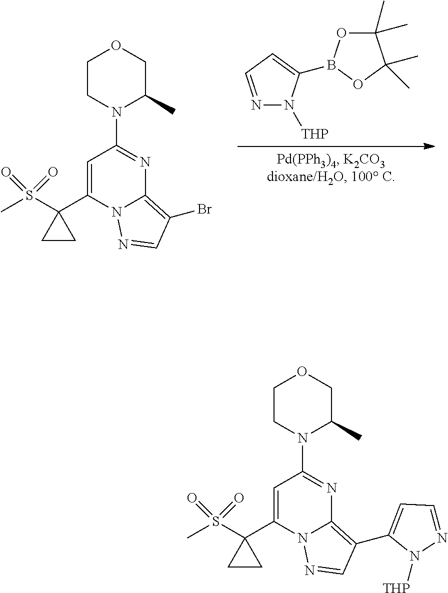

To a solution of (R)-4-(3-bromo-7-(1-(methylsulfonyl)cyclopropyl)pyrazolo[1,5-a] pyrimidin-5-yl)-3-methylmorpholine (170 mg, 0.41 mmol) and 1-(oxan-2-yl)-5-(tetramethyl-1,3,2-dioxaborolan-2-yl)-1H-pyrazole (227.7 mg, 0.82 mmol) in co-solvent of dioxane (10 mL) and H₂O (2 mL) were added K₂CO₃ (141.4 mg, 1.02 mmol) and Pd(PPh₃)₄ (47.28 mg, 0.041 mmol). The mixture was stirred at 100° C. for 6 h under nitrogen atmosphere. LC-MS showed the reaction was complete. The reaction mixture was diluted with EA (50 mL), then washed with water and brine, dried over anhydrous Na₂SO₄, filtered and concentrated. The residue was purified by column chromatography on silica gel (PE:EA=1:1, V/V) to give the desired product (150 mg, yield: 75%). LC/MS (ESI) m/z: 487 [M+H]⁺.

### Step 5. (R)-3-methyl-4-(7-(1-(methylsulfonyl)cyclopropyl)-3-(1H-pyrazol-5-yl) pyrazolo[1,5-a]pyrimidin-5-yl)morpholine

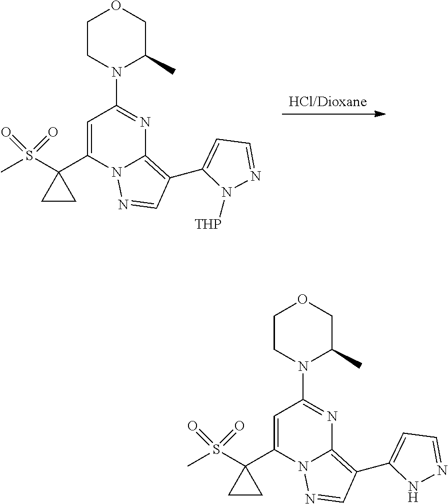

To a solution of (3R)-3-methyl-4-(7-(1-(methylsulfonyl)cyclopropyl)-3-(1-(tetrahydro-2H-pyran-2-yl)-1H-pyrazol-5-yl)pyrazolo[1,5-a]pyrimidin-5-yl)morpholine (120 mg, 0.25 mmol) in DCM (3 mL) was added HCl solution (4M in dioxane, 3 mL). The mixture was stirred at room temperature for 1 h. LC-MS showed the reaction was complete. The reaction mixture was concentrated under vacuo. The residue was purified by Pre-HPLC (C₁₈, 10-95%, MeOH in H₂O with 0.1% HCOOH) to afford the desired product (35 mg, yield: 35%). LC/MS (ESI) m/z: 403 [M+H]+. ¹H NMR (400 MHz, DMSO) δ 12.74 (d, J=87.9 Hz, 1H), 8.32 (s, 1H), 7.52 (s, 1H), 6.98 (s, 1H), 6.71 (s, 1H), 4.58 (s, 1H), 4.22 (s, 1H), 4.00 (dd, J=11.4, 3.1 Hz, 1H), 3.79 (d, J=11.5 Hz, 1H), 3.66 (dd, J=11.4, 2.8 Hz, 1H), 3.51 (td, J=11.7, 2.7 Hz, 1H), 3.29-3.20 (m, 1H), 3.16 (s, 3H), 1.93-1.83 (m, 2H), 1.65 (q, J=5.7 Hz, 2H), 1.25 (t, J=11.2 Hz, 3H).

### Example 6

### Synthesis of (R)-4-(7-(2-fluoropyridin-3-yl)-3-(1H-pyrazol-5-yl)pyrazolo[1,5-a]pyrimidin-5-yl)-3-methylmorpholine

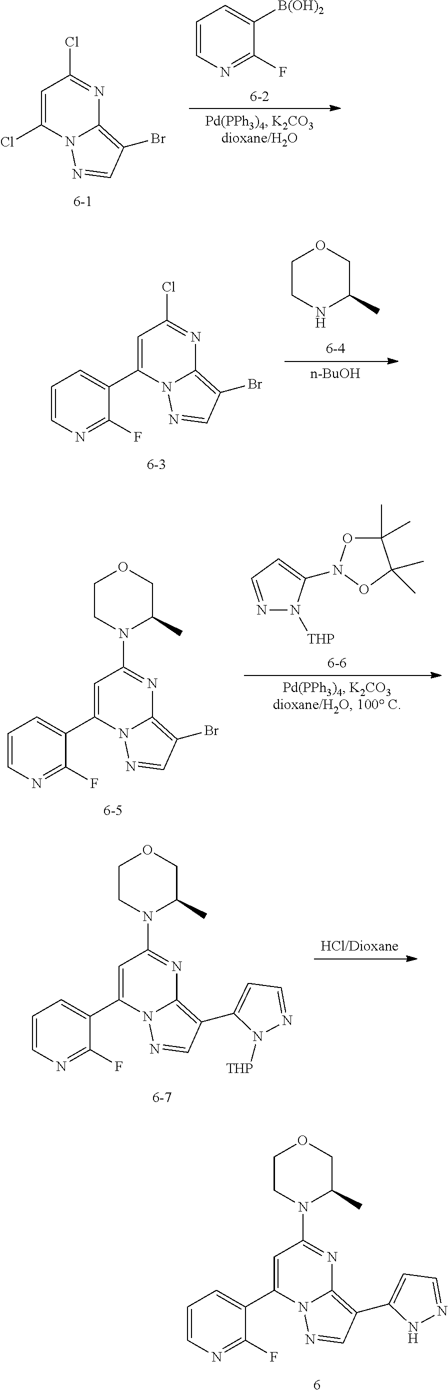

### Step 1. 3-bromo-5-chloro-7-(2-fluoropyridin-3-yl)pyrazolo[1,5-a]pyrimidine

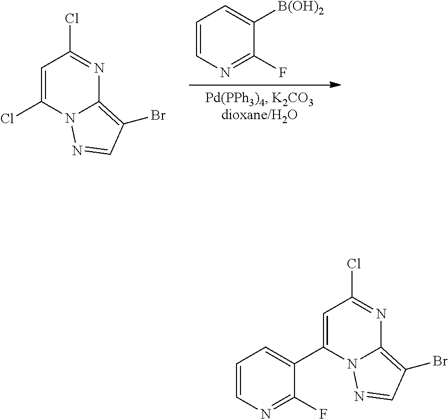

To a solution of 3-bromo-5,7-dichloropyrazolo[1,5-a]pyrimidine (0.46 mL, 3.75 mmol) and (2-fluoropyridin-3-yl)boronic acid (2.20 g, 7.49 mmol) in co-solvent of dioxane (50 mL) and H₂O (10 mL) were added K₂CO₃ (1.29 g, 9.37 mmol) and Pd(PPh₃)₄ (0.43 g, 0.38 mmol). The mixture was stirred at 90° C. overnight under nitrogen atmosphere. The reaction was diluted with EA (60 mL), then washed with water and brine, dried over anhydrous Na₂SO₄, filtered and concentrated. The residue was purified by column chromatography on silica gel (PE:EA=3:1, V/V) to afford the desired product (650 mg, yield: 53%). LC/MS (ESI) m/z: 327/329 [M+H]⁺. ¹H NMR (400 MHz, DMSO) δ 8.54 (dd, J=4.9, 0.9 Hz, 1H), 8.47 (s, 1H), 8.43 (ddd, J=9.4, 7.5, 1.9 Hz, 1H), 7.69-7.63 (m, 1H), 7.61 (s, 1H).

### Step 2. (R)-4-(3-bromo-7-(2-fluoropyridin-3-yl)pyrazolo[1,5-a]pyrimidin-5-yl)-3-methylmorpholine

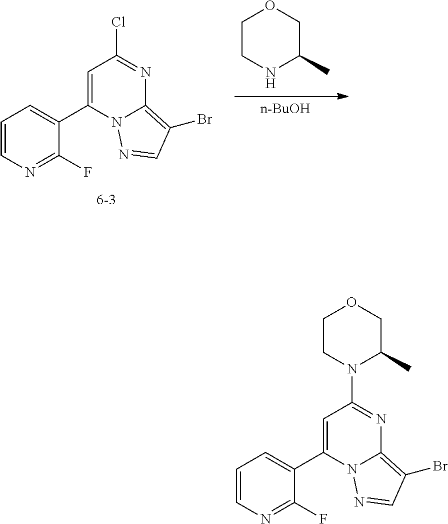

To a solution of 3-bromo-5-chloro-7-(2-fluoropyridin-3-yl)pyrazolo[1,5-a]pyrimidine (300 mg, 0.92 mmol) in n-BuOH (10 mL) was added (3R)-3-methylmorpholine (833.8 mg, 8.24 mmol). The reaction was stirred at 145° C. for 1 h under microwave irradiation. LC-MS showed the reaction was complete. The mixture was diluted with EA (60 mL), then washed with water and brine, dried over anhydrous Na₂SO₄, filtered and concentrated. The residue was purified by column chromatography on silica gel (PE:EA=2:1, V/V) to afford the desired product (280 mg, yield: 78%). LC/MS (ESI) m/z: 392/394 [M+H]⁺. ¹H NMR (400 MHz, DMSO) δ 8.47 (dt, J=20.7, 10.4 Hz, 1H), 8.33 (ddd, J=9.4, 7.4, 1.9 Hz, 1H), 7.98 (s, 1H), 7.60 (ddd, J=7.1, 4.9, 1.9 Hz, 1H), 7.05 (s, 1H), 4.54 (d, J=6.2 Hz, 1H), 4.21 (d, J=14.8 Hz, 1H), 4.02-3.92 (m, 1H), 3.76 (d, J=11.5 Hz, 1H), 3.64 (dd, J=11.5, 3.0 Hz, 1H), 3.49 (td, J=11.9, 2.9 Hz, 1H), 3.30-3.20 (m, 1H), 1.26 (d, J=6.7 Hz, 3H).

### Step 3. (3R)-4-(7-(2-fluoropyridin-3-yl)-3-(1-(tetrahydro-2H-pyran-2-yl)-1H-pyrazol-5-yl)pyrazolo[1,5-a]pyrimidin-5-yl)-3-methylmorpholine

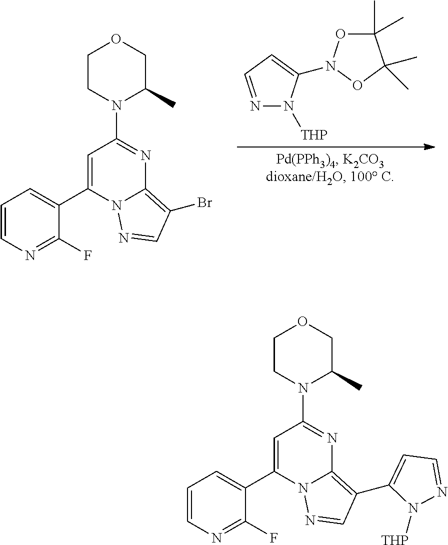

To a solution of (R)-4-(3-bromo-7-(2-fluoropyridin-3-yl)pyrazolo[1,5-a]pyrimidin-5-yl)-3-methylmorpholine (140 mg, 0.36 mmol) and 1-(oxan-2-yl)-5-(tetramethyl-1,3,2-dioxaborolan-2-yl)-1H-pyrazole (198.6 mg, 0.71 mmol) in co-solvent of dioxane (10 mL) and H₂O (2 mL) were added K₂CO₃ (123.3 mg, 0.89 mmol) and Pd(Phh₃)₄ (41.2 mg, 0.04 mmol). The mixture was stirred at 100° C. overnight under nitrogen atmosphere. LC-MS showed the reaction was complete.

The reaction was diluted with EA (50 mL), then washed with water and brine, dried over anhydrous Na₂SO₄, filtered and concentrated. The residue was purified by column chromatography on silica gel (PE:EA=1:1, V/V) to afford the desired product (120 mg, yield: 72%). LC/MS (ESI) m/z: 464 [M+H]⁺.

### Step 4. (R)-4-(7-(2-fluoropyridin-3-yl)-3-(1H-pyrazol-5-yl)pyrazolo[1,5-a]pyrimidin-5-yl)-3-methylmorpholine

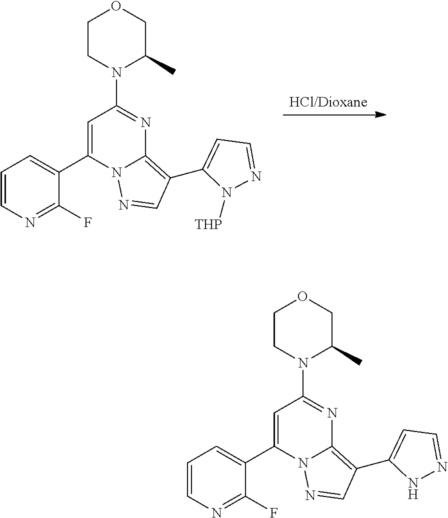

A mixture of (3R)-4-(7-(2-fluoropyridin-3-yl)-3-(1-(tetrahydro-2H-pyran-2-yl)-1H-pyrazol-5-yl)pyrazolo[1,5-a]pyrimidin-5-yl)-3-methylmorpholine (120 mg, 0.26 mmol) in HCl solution (4M in dioxane, 3 mL) was stirred at room temperature for 2 h. LC-MS showed the reaction was complete. The reaction mixture was concentrated under vacuo. The residue was purified by Pre-HPLC (C₁₈, 10-95%, MeOH in H₂O with 0.1% HCOOH) to afford the desired product (20 mg, yield: 20%). LC/MS (ESI) m/z: 380 [M+H]⁺. ¹H NMR (400 MHz, DMSO) δ 8.49 (dd, J=4.9, 1.1 Hz, 1H), 8.37 (ddd, J=9.4, 7.4, 1.9 Hz, 1H), 8.25 (d, J=6.6 Hz, 1H), 7.62 (ddd, J=7.1, 4.9, 1.8 Hz, 2H), 7.02 (s, 1H), 6.76 (s, 1H), 4.58 (s, 1H), 4.26 (d, J=12.7 Hz, 1H), 4.01 (dd, J=11.4, 3.4 Hz, 1H), 3.79 (d, J=11.4 Hz, 1H), 3.67 (dd, J=11.4, 2.9 Hz, 1H), 3.53 (td, J=11.8, 2.8 Hz, 1H), 3.26 (s, 1H), 1.29 (d, J=6.7 Hz, 3H). ¹H NMR (400 MHz, MeOD) S 8.42 (dd, J=4.9, 1.0 Hz, 1H), 8.28 (ddd, J=9.3, 7.5, 1.9 Hz, 1H), 8.23 (d, J=4.6 Hz, 1H), 7.60 (dd, J=11.3, 2.3 Hz, 1H), 7.55-7.47 (m, 1H), 6.85 (d, J=2.0 Hz, 1H), 6.81 (d, J=11.1 Hz, 1H), 4.59 (d, J=4.2 Hz, 1H), 4.24 (d, J=13.4 Hz, 1H), 4.05 (dd, J=11.4, 3.6 Hz, 1H), 3.84 (d, J=11.5 Hz, 1H), 3.78 (dd, J=11.6, 2.9 Hz, 1H), 3.64 (td, J=12.0, 3.0 Hz, 1H), 3.40 (td, J=12.9, 3.8 Hz, 1H), 1.39 (d, J=6.8 Hz, 1H).

### Example 7

### Synthesis of imino(methyl)(l-(5-((R)-3-methylmorpholino)-3-(1H-pyrazol-5-yl)pyrazolo[1,5-a]pyrimidin-7-yl)cyclopropyl)-λ6-sulfanone

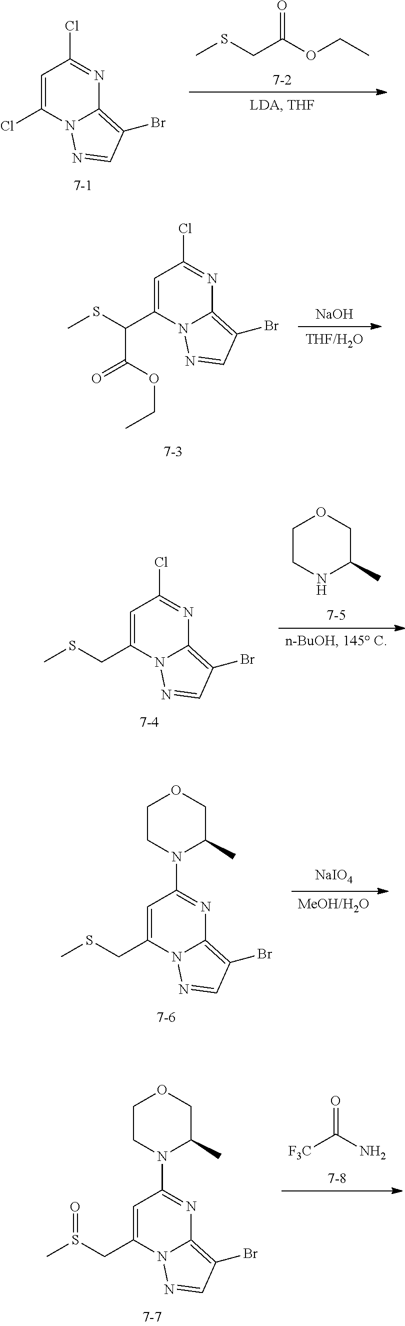

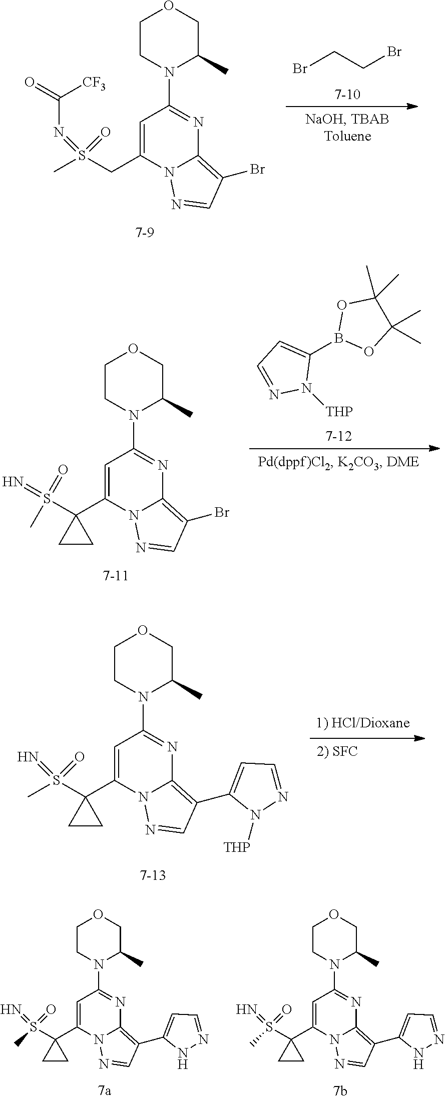

### Step 1. ethyl 2-(3-bromo-5-chloropyrazolo[1,5-a]pyrimidin-7-yl)-2-(methylthio) acetate

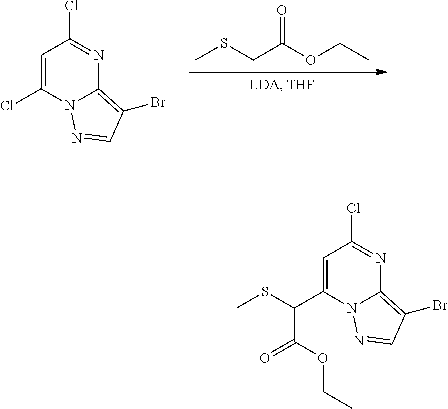

To a solution of ethyl 2-(methylsulfanyl)acetate (1 g, 7.49 mmol) in THF (30 mL) at −60° C. was added LDA (2 M in THF, 4.68 mL, 9.37 mmol) drop wise. The mixture was stirred at −60° C. for 1 h, then a solution of 3-bromo-5,7-dichloropyrazolo [1,5-a]pyrimidine (1 g, 3.75 mmol) in THF (2 mL) was added drop wise. The resulting mixture was stirred at −60° C. for an additional 1 h. LC-MS showed the reaction was complete. The reaction mixture was quenched with saturated NH₄Cl aqueous solution, then extracted with EA (30 mL×3). The combined organic layer was washed with brine, dried over anhydrous Na₂SO₄, filtered and concentrated. The residue was purified by column chromatography on silica gel (PE:EA=50:1, V/V) to afford the desired product (1.2 g, yield: 87%). LC/MS (ESI) m/z: 364/396 [M+H]⁺.

### Step 2. 3-bromo-5-chloro-7-((methylthio)methyl)pyrazolo[1,5-a]pyrimidine

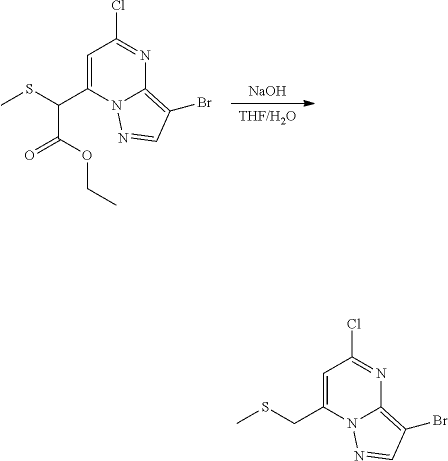 [SMILES: CCOC(=O)C(SC)c1cc(Cl)nc2c(Br)cnn12 ; "NaOH" ; "THF/H2O" ; CSCc1cc(Cl)nc2c(Br)cnn12]

To a solution of ethyl 2-(3-bromo-5-chloropyrazolo[1,5-a]pyrimidin-7-yl)-2-(methylthio)acetate (1.2 g, 3.29 mmol) in co-solvent of THF (40 mL) and H₂O (12 mL) was added NaOH (0.39 g, 9.87 mmol). The mixture was stirred at 60° C. for 30 min. LC-MS showed the reaction was complete. The reaction was diluted with EA (50 mL), then washed with water and brine, dried over anhydrous Na₂SO₄, filtered and concentrated. The residue was purified by column chromatography on silica gel (PE:EA=50:1, V/V) to afford the desired product (670 mg, yield: 69%). LC/MS (ESI) m/z: 292/294 [M+H]⁺.

### Step 3. (R)-4-(3-bromo-7-((methylthio)methyl)pyrazolo[1,5-a]pyrimidin-5-yl)-3-methylmorpholine

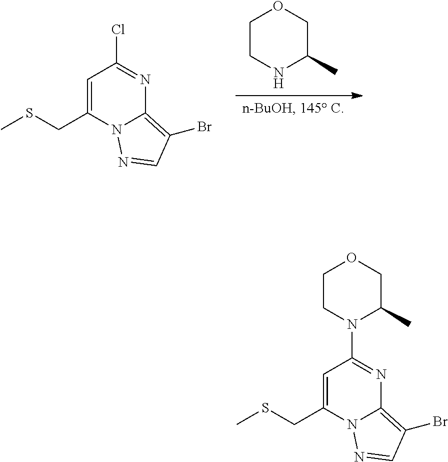

To a solution of 3-bromo-5-chloro-7-((methylthio)methyl)pyrazolo[1,5-a]pyrimidine (670 mg, 2.29 mmol) in n-BuOH (10 mL) were added (3R)-3-methylmorpholine (2.08 g, 20.61 mmol). The mixture was stirred at 145° C. for 1 h under microwave irradiation. LC-MS showed reaction was complete. The mixture was diluted with EA (50 mL), then washed with water and brine, dried over anhydrous Na₂SO₄, filtered and concentrated. The residue was purified by column chromatography on silica gel (PE:EA=5:1, V/V) to afford the desired product (730 mg, yield: 89%). LC/MS (ESI) m/z: 357/359 [M+H]⁺.

### Step 4. (3R)-4-(3-bromo-7-((methylsulfinyl)methyl)pyrazolo[1,5-a]pyrimidin-5-yl)-3-methylmorpholine

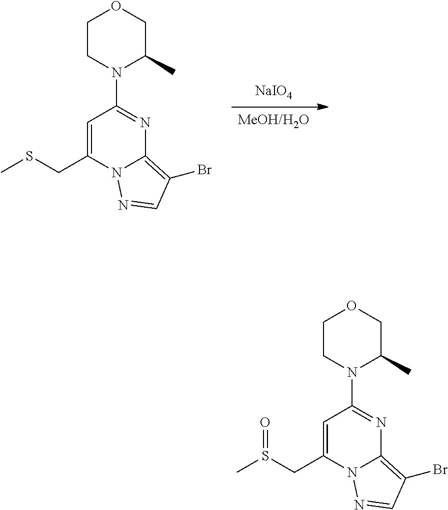 [SMILES: CSCc1cc(N2CCOC[C@H]2C)nc2c(Br)cnn12 ; "NaIO4" ; "MeOH/H2O" ; C[C@@H]1COCCN1c1cc(CS(C)=O)n2ncc(Br)c2n1]

To a solution of (R)-4-(3-bromo-7-((methylthio)methyl)pyrazolo[1,5-a]pyrimidin-5-yl)-3-methylmorpholine (730 mg, 2.04 mmol) in co-solvent of MeOH (25 mL) and H₂O (5 mL) was added sodium periodate (437.0 mg, 2.04 mmol). The mixture was stirred at room temperature overnight. LC-MS showed reaction was complete. The reaction was diluted with DCM (50 mL), then washed with water and brine, dried over anhydrous Na₂SO₄, filtered and concentrated. The residue was purified by column chromatography on silica gel (DCM:MeOH=50:1, V/V) to afford the desired product (680 mg, yield: 89%). LC/MS (ESI) m/z: 373/375 [M+H]⁺.

### Step 5. ((3-bromo-5-((R)-3-methylmorpholino)pyrazolo[1,5-a]pyrimidin-7-yl)methyl)(methyl)((2,2,2-trifluoroethyl)imino)-16-sulfanone

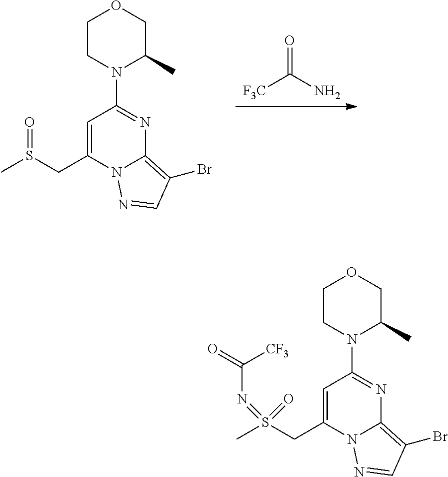 [SMILES: C[C@@H]1COCCN1c1cc(CS(C)=O)n2ncc(Br)c2n1 ; NC(=O)C(F)(F)F ; C[C@@H]1COCCN1c1cc(CS(C)(=O)=NC(=O)C(F)(F)F)n2ncc(Br)c2n1]

To a solution of (3R)-4-(3-bromo-7-((methylsulfinyl)methyl)pyrazolo[1,5-a]pyrimidin-5-yl)-3-methylmorpholine (680 mg, 1.82 mmol) and trifluoroacetamide (411.8 mg, 3.64 mmol) in DCM (30 mL) were added MgO (293.6 mg, 7.28 mmol), (Diacetoxyiodo)benzene (880.1 mg, 2.73 mmol) and Rhodium acetate (12.7 mg, 0.046 mmol). The mixture was stirred at room temperature overnight under nitrogen atmosphere. LC-MS showed reaction was complete. The reaction mixture was diluted with DCM (50 mL), then washed with water and brine, dried over anhydrous Na₂SO₄, filtered and concentrated. The residue was purified by column chromatography on silica gel (PE:EA=3:1, V/V) to afford the desired product (500 mg, yield: 56%). LC/MS (ESI) m/z: 484/486 [M+H]⁺.

### Step 6. (1-(3-bromo-5-((R)-3-methylmorpholino)pyrazolo[1,5-a]pyrimidin-7-yl)cyclopropyl)(imino)(methyl)-λ6-sulfanone

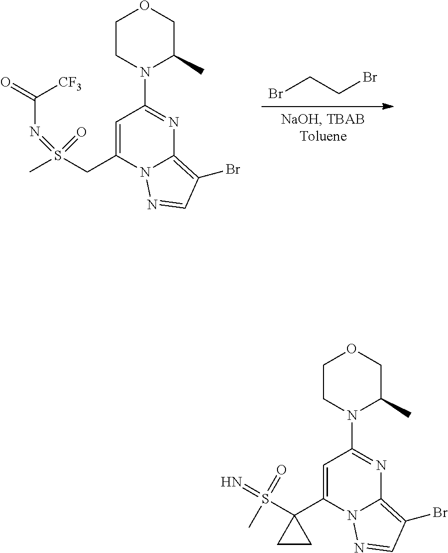

To a solution of N-[({3-bromo-5-[(3R)-3-methylmorpholin-4-yl]pyrazolo[1,5-a] pyrimidin-7-yl}methyl)(methyl)oxo-λ6-sulfanylidene]-2,2,2-trifluoroacetamide (400 mg, 0.83 mmol) in Toluene (20 mL) were added 1,2-dibromoethane (388 mg, 2.07 mmol), NaOH (10 M in H₂O, 0.83 mL, 8.26 mmol) and TBAB (54 mg, 0.17 mmol). The mixture was stirred at 60° C. overnight. LC-MS showed reaction was complete. The reaction was diluted with EA (50 mL), then washed with water and brine, dried over anhydrous Na₂SO₄, filtered and concentrated. The residue was purified by column chromatography on silica gel (DCM:MeOH=30:1, V/V) to afford the desired product (140 mg, yield: 40%). LC/MS (ESI) m/z: 414/416 [M+H]⁺.

### Step 7. Imino(methyl)(l-(5-((R)-3-methylmorpholino)-3-(1-(tetrahydro-2H-pyran-2-yl)-1H-pyrazol-5-yl)pyrazolo[1,5-a]pyrimidin-7-yl)cyclopropyl)-λ6-sulfanone

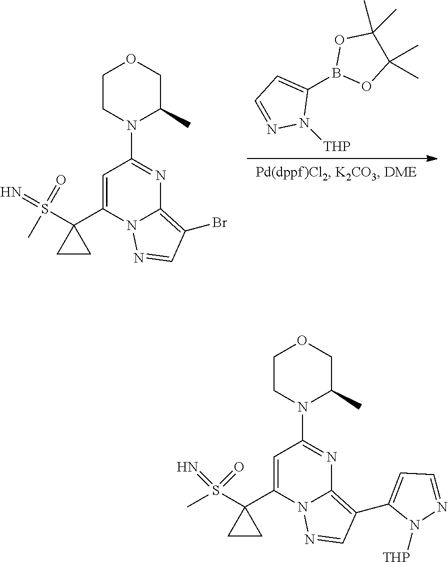

To a solution of (1-(3-bromo-5-((R)-3-methylmorpholino)pyrazolo[1,5-a]pyrimidin-7-yl)cyclopropyl)(imino)(methyl)-λ6-sulfanone (130 mg, 0.31 mmol) and 1-(oxan-2-yl)-5-(tetramethyl-1,3,2-dioxaborolan-2-yl)-1H-pyrazole (174.6 mg, 0.62 mmol) in DME (5 mL) were added K₂CO₃ (107.8 mg, 0.78 mmol) and Pd(dppf)Cl₂ (22.96 mg, 0.031 mmol). The mixture was stirred at 90° C. overnight under nitrogen atmosphere. LC-MS showed reaction was complete. The reaction was diluted with EA (50 mL), then washed with water and brine, dried over anhydrous Na₂SO₄, filtered and concentrated. The residue was purified by column chromatography on silica gel (PE:EA=1:1, V/V) to afford the desired product (45 mg, yield: 29%). LC/MS (ESI) m/z: 486 [M+H]⁺.

### Step 8. Imino(methyl)(l-(5-((R)-3-methylmorpholino)-3-(1H-pyrazol-5-yl)pyrazolo [1,5-a]pyrimidin-7-yl)cyclopropyl)-λ6-sulfanone

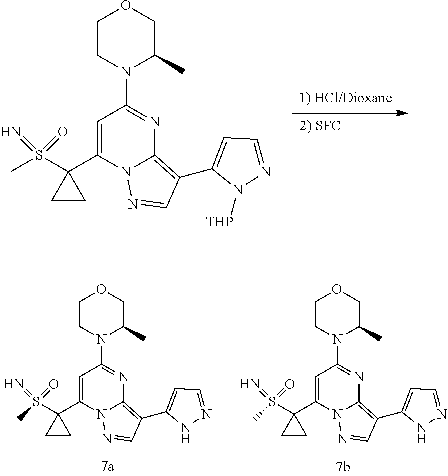

A solution of imino(methyl)(1-(5-((R)-3-methylmorpholino)-3-(1-(tetrahydro-2H-pyran-2-yl)-1H-pyrazol-5-yl)pyrazolo[1,5-a]pyrimidin-7-yl)cyclopropyl)-λ6-sulfanone (40 mg, 0.08 mmol) in DCM (2 mL) was added HCl solution (4M in dioxane, 2 mL). The mixture was stirred at room temperature for 30 min. LC-MS showed the reaction was complete. The reaction mixture was concentrated under vacuo. The residue was purified by Prep-HPLC (C₁₈, 10-95%, MeOH in H₂O with 0.1% HCOOH) to obtain the diastereomer (20 mg), which was further separated by SFC (Chiral column OJ-H 4.6×250 mm, 5 μm; pump A: SF CO₂, pump B: MeOH+0.05% DEA, 5%-40%, 8.5 min) to afford the (R)-imino(methyl)(1-(5-((R)-3-methylmorpholino)-3-(1H-pyrazol-5-yl)pyrazolo[1,5-a]pyrimidin-7-yl)cyclopropyl)-λ6-sulfanone (0.8 mg, yield: 2.4%) and (S)-imino(methyl)(1-(5-((R)-3-methylmorpholino)-3-(1H-pyrazol-5-yl)pyrazolo[1,5-a] pyrimidin-7-yl)cyclopropyl)-λ6-sulfanone (2.5 mg, yield: 7.5%). LC/MS (ESI) m/z: 402 [M+H]⁺. ¹H NMR (400 MHz, DMSO) δ 8.31 (s, 1H), 7.59 (s, 1H), 6.97 (s, 1H), 6.72 (s, 1H), 4.57 (d, J=5.8 Hz, 1H), 4.22 (d, J=12.9 Hz, 1H), 4.01 (dd, J=11.3, 3.2 Hz, 1H), 3.87-3.76 (m, 2H), 3.66 (dd, J=11.4, 2.8 Hz, 1H), 3.52 (dd, J=11.9, 2.8 Hz, 1H), 3.01 (s, 3H), 1.79 (dd, J=14.9, 10.4, 4.2 Hz, 2H), 1.59-1.45 (m, 2H), 1.27 (d, J=6.7 Hz, 3H).

### Example 8

### Synthesis of (R)-3-methyl-4-(7-(1-(methylsulfonyl)cyclopropyl)-3-(1H-pyrrol-2-yl)pyrazolo[1,5-a]pyrimidin-5-yl)morpholine

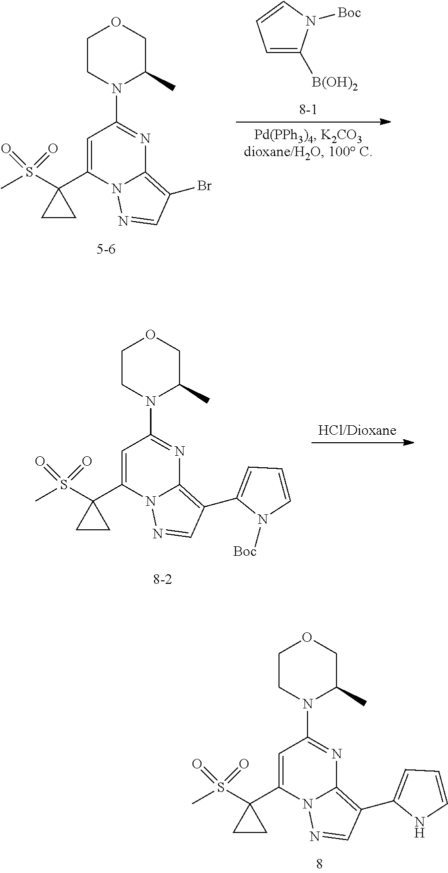

### Step 1. Tert-butyl(R)-2-(5-(3-methylmorpholino)-7-(1-(methylsulfonyl)cyclopropyl) pyrazolo[1,5-a]pyrimidin-3-yl)-1H-pyrrole-1-carboxylate

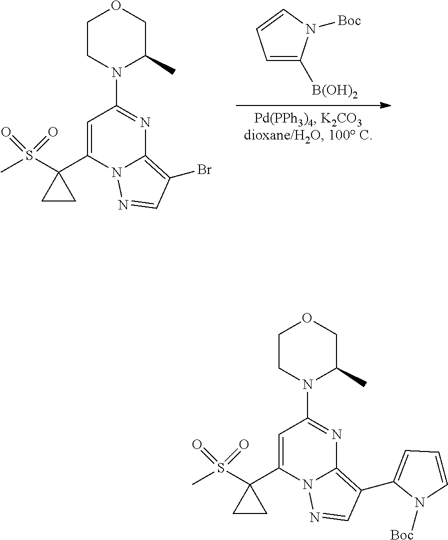

A mixture of (3R)-4-[3-bromo-7-(1-(methanesulfonyl)cyclopropyl)pyrazolo[1,5-a] pyrimidin-5-yl]-3-methylmorpholine (128 mg, 0.30 mmol), {1-[(tert-butoxy) carbonyl]-1H-pyrrol-2-yl}boronic acid (130 mg, 0.62 mmol), Pd(PPh₃)₄ (35.6 mg, 0.03 mmol) and K₂CO₃ (107 mg, 0.77 mmol) in co-solvent of dioxane (5 mL) and H₂O (1 mL) was stirred at 100° C. for 16 h under N₂ atmosphere. LC-MS showed the reaction was complete. The reaction mixture was diluted with EA (40 mL), then washed with water and brine, dried over anhydrous Na₂SO₄, filtered and concentrated. The residue was purified by flash chromatography on silica gel (PE:EA=5:1, V/V) to give the desired product (71 mg, yield: 45%). LC/MS (ESI): m/z 502 [M+H]⁺.

### Step 2. (R)-3-methyl-4-(7-(1-(methylsulfonyl)cyclopropyl)-3-(1H-pyrrol-2-yl)pyrazolo[1,5-a]pyrimidin-5-yl)morpholine

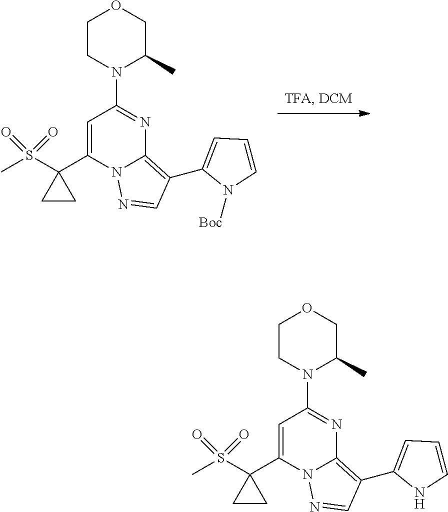

To a solution of tert-butyl 2-[7-(1-methanesulfonylcyclopropyl)-5-[(3R)-3-methyl morpholin-4-yl]pyrazolo[1,5-a]pyrimidin-3-yl]-1H-pyrrole-1-carboxylate (71 mg, 0.14 mmol) in DCM (5 mL) was added TFA (2 mL). The mixture was stirred at room temperature for 4 h. LC-MS showed the reaction was complete. The reaction mixture was concentrated under vacuo to dryness. The residue was purified by Prep-HPLC (C₁₈, 10-95%, MeOH in H₂O with 0.1% HCOOH) to give the desired product (32 mg, yield: 56%). LC/MS (ESI): m/z 402 [M+H]⁺. ¹H NMR (400 MHz, DMSO) δ 10.78 (s, 1H), 8.26 (s, 1H), 6.93 (s, 1H), 6.77-6.71 (m, 1H), 6.49-6.47 (m, 1H), 6.08 (m, 6.09-6.07, 2.6 Hz, 1H), 4.58-4.56 (m, 1H), 4.22 (d, J=12.9 Hz, 1H), 4.00 (dd, J=11.3, 3.1 Hz, 1H), 3.79 (d, J=11.4 Hz, 1H), 3.66 (dd, J=11.4, 2.8 Hz, 1H), 3.57-3.46 (m, 1H), 3.28-3.20 (m, 1H), 3.15 (s, 3H), 2.08 (s, 1H), 1.88 (q, J=5.4 Hz, 2H), 1.63 (q, J=5.7 Hz, 2H), 1.26 (d, J=6.7 Hz, 3H).

### Example 9

### Synthesis of (R)-3-methyl-4-(7-(1-(methylsulfonyl)cyclopropyl)-3-(1H-pyrrol-3-yl)pyrazolo[1,5-a]pyrimidin-5-yl)morpholine

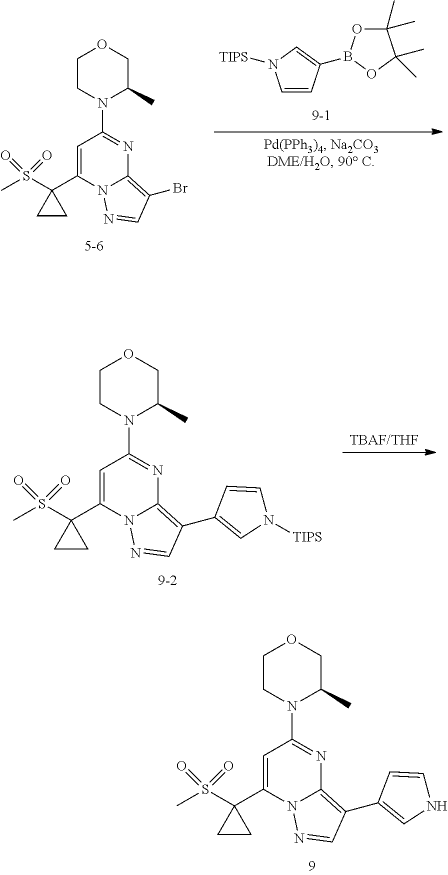

### Step 1. (R)-3-methyl-4-(7-(1-(methylsulfonyl)cyclopropyl)-3-(1-(triisopropylsilyl)-1H-pyrrol-3-yl)pyrazolo[1,5-a]pyrimidin-5-yl)morpholine

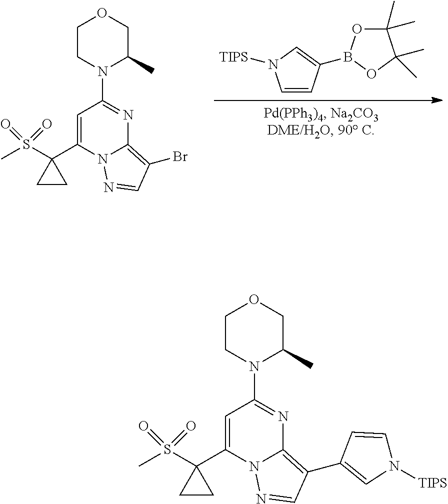

A mixture of (3R)-4-[3-bromo-7-(1-methanesulfonylcyclopropyl)pyrazolo[1,5-a] pyrimidin-5-yl]-3-methylmorpholine (100 mg, 0.24 mmol), 3-(tetramethyl-1,3,2-dioxaborolan-2-yl)-1-[tris(propan-2-yl)silyl]-1H-pyrrole (168 mg, 0.48 mmol), Pd(PPh₃)₄ (27.8 mg, 0.024 mmol) and Na₂CO₃ (76 mg, 0.72 mmol) in co-solvent of DME (3 mL) and H₂O (0.6 mL) was stirred at 90° C. for 16 h under N₂ atmosphere. LC-MS showed the reaction was complete. The reaction mixture was diluted with EA (40 mL), then washed with water and brine, dried over anhydrous Na₂SO₄, filtered and concentrated. The residue was purified by flash chromatography on silica gel (PE:EA=2:1, V/V) to give the desired product (49 mg, yield: 36%). LC/MS (ESI): m/z 558 [M+H]⁺.

### Step 2. (R)-3-methyl-4-(7-(1-(methylsulfonyl)cyclopropyl)-3-(1H-pyrrol-3-yl)pyrazolo[1,5-a]pyrimidin-5-yl)morpholine

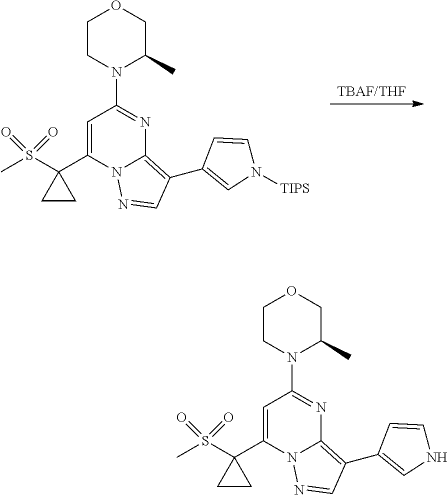

A mixture of (3R)-4-[7-(1-methanesulfonylcyclopropyl)-3-{1-[tris(propan-2-yl)silyl]-1H-pyrrol-3-yl}pyrazolo[1,5-a]pyrimidin-5-yl]-3-methylmorpholine (44 mg, 0.07 mmol) and TBAF (1.0 M in THF, 0.15 mL) in THF (5 mL) was stirred at room temperature for 0.5 h. LC-MS showed the reaction was complete. The reaction mixture was diluted with EA (40 mL), then washed with water and brine, dried over anhydrous Na₂SO₄, filtered and concentrated. The residue was purified by Prep-HPLC (C₁₈, 10-95%, MeOH in H₂O with 0.1% HCOOH) to give the desired product (14.7 mg, yield: 46%). LC/MS (ESI): m/z 402 [M+H]⁺. ¹H NMR (400 MHz, DMSO) δ 10.69 (s, 1H), 8.15 (s, 1H), 7.22 (s, 1H), 6.89 (s, 1H), 6.76 (d, J=2.1 Hz, 1H), 6.52 (d, J=1.4 Hz, 1H), 4.52 (d, J=5.3 Hz, 1H), 4.14 (d, J=12.5 Hz, 1H), 4.03-3.95 (m, 1H), 3.78 (d, J=11.4 Hz, 1H), 3.66 (dd, J=11.3, 2.6 Hz, 1H), 3.51 (t, J=10.6 Hz, 1H), 3.28-3.19 (m, 1H), 3.16 (s, 3H), 1.87 (q, J=5.5 Hz, 2H), 1.62 (q, J=5.8 Hz, 2H), 1.25 (d, J=6.7 Hz, 3H).

### Example 10

### Synthesis of (R)-4-(3,7-di(1H-pyrazol-5-yl)pyrazolo[1,5-a]pyrimidin-5-yl)-3-methylmorpholine

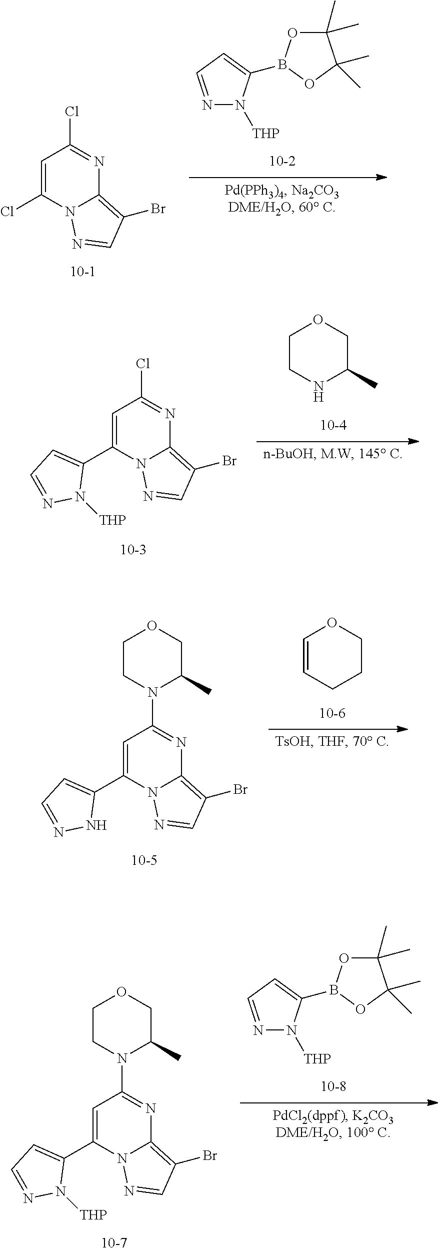

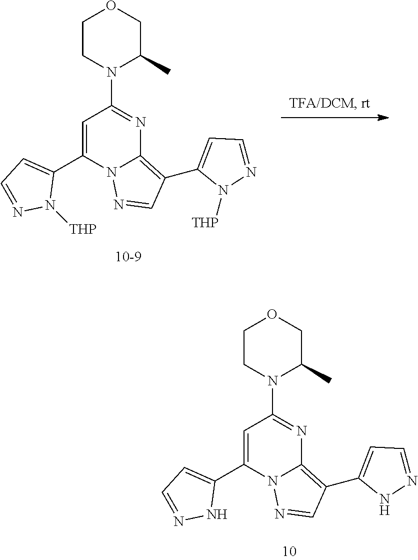

### Step 1. 3-bromo-5-chloro-7-(1-(tetrahydro-2H-pyran-2-yl)-1H-pyrazol-5-yl)pyrazolo [1,5-a]pyrimidine

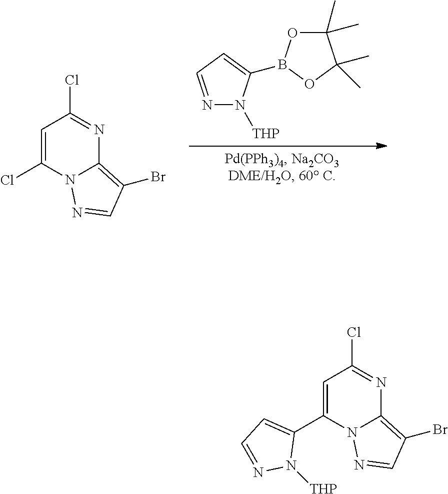

A mixture of 3-bromo-5,7-dichloropyrazolo[1,5-a]pyrimidine (400 mg, 1.49 mmol), 1-(oxan-2-yl)-5-(tetraMethyl-1,3,2-dioxaborolan-2-yl)-1H-pyrazole (420 mg, 1.51 mmol), Pd(PPh₃)₄ (87 mg, 0.075 mmol) and Na₂CO₃ (320 mg, 3.01 mmol) in co-solvent of DME (20 mL) and H₂O (4 mL) was stirred at 60° C. for 4 h under N₂ atmosphere. LC-MS showed the reaction was complete. The mixture was diluted with EA (40 mL), then washed with water and brine, dried over anhydrous Na₂SO₄, filtered and concentrated. The residue was purified by flash chromatography on silica gel (PE:EA=10:1, V/V) to afford the desired product (376 mg, yield: 65%). LC/MS (ESI): m/z 382/384 [M+H]⁺.

### Step 2. (R)-4-(3-bromo-7-(1H-pyrazol-5-yl)pyrazolo[1,5-a]pyrimidin-5-yl)-3-methylmorpholine

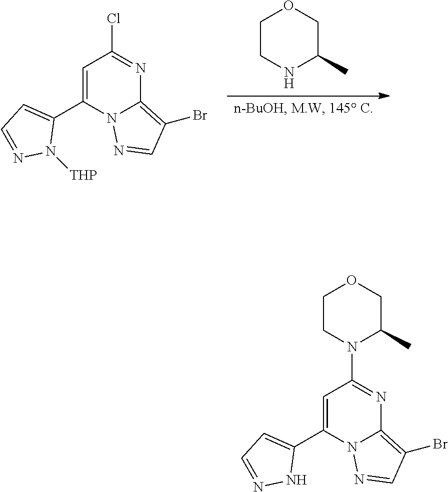

A mixture of 5-{3-bromo-5-chloropyrazolo[1,5-a]pyrimidin-7-yl}-1-(oxan-2-yl)-1H-pyrazole (100 mg, 0.26 mmol) and (3R)-3-methylmorpholine (238 mg, 2.35 mmol) in n-BuOH (3 mL) was stirred at 145° C. for 1 h under microwave irradiation. LC-MS showed the reaction was complete. The reaction mixture was diluted with EA (40 mL), then washed with water and brine, dried over anhydrous Na₂SO₄, filtered and concentrated. The residue was purified by flash chromatography on silica gel (PE:EA=3:1, V/V) to afford the desired product (66 mg, yield: 69%). LC/MS (ESI): m/z 363/365 [M+H]⁺.

### Step 3. (3R)-4-(3-bromo-7-(1-(tetrahydro-2H-pyran-2-yl)-1H-pyrazol-5-yl)pyrazolo[1,5-a]pyrimidin-5-yl)-3-methylmorpholine

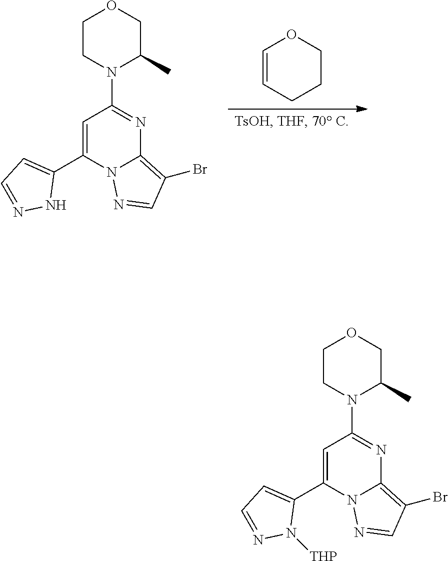

A mixture of (3R)-4-[3-bromo-7-(1H-pyrazol-5-yl)pyrazolo[1,5-a]pyrimidin-5-yl]-3-methylmorpholine (60 mg, 0.16 mmol), 3,4-dihydro-2H-pyran (64 mg, 0.76 mmol) and 4-methylbenzenesulfonic acid (6 mg, 0.03 mmol) in THF (5 mL) was stirred at 70° C. for 5 h. LC-MS showed the reaction was complete. The reaction mixture was diluted with EA (40 mL), then washed with water and brine, dried over anhydrous Na₂SO₄, filtered and concentrated. The residue was purified by flash chromatography on silica gel (PE:EA=3:1, V/V) to afford the desired product (72 mg, yield: 97%). LC/MS (ESI): m/z 447 [M+H]⁺.

### Step 4. (3R)-4-(3,7-bis(1-(tetrahydro-2H-pyran-2-yl)-1H-pyrazol-5-yl)pyrazolo[1,5-a]pyrimidin-5-yl)-3-methylmorpholine

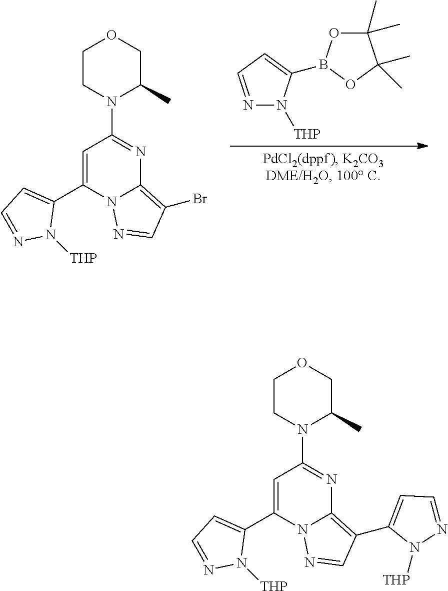

A mixture of (3R)-4-{3-bromo-7-[1-(oxan-2-yl)-1H-pyrazol-5-yl]pyrazolo[1,5-a] pyrimidin-5-yl}-3-methylmorpholine (72 mg, 0.16 mmol), 1-(oxan-2-yl)-5-(tetra methyl-1,3,2-dioxaborolan-2-yl)-1H-pyrazole (89.7 mg, 0.32 mmol), Pd(dppf)Cl₂ (11.7 mg, 0.016 mmol) and K₂CO₃ (55.5 mg, 0.40 mmol) in co-solvent of DME (3 mL) and H₂O (0.6 mL) was stirred at 100° C. for 5 h under N₂ atmosphere. LC-MS showed the reaction was complete. The reaction mixture was diluted with EA (40 mL), then washed with water and brine, dried over anhydrous Na₂SO₄, filtered and concentrated. The residue was purified by flash chromatography on silica gel (PE:EA=1:1, V/V) to afford the desired product (47 mg, yield: 67%). LC/MS (ESI): m/z 519 [M+H]⁺.

### Step 5. (R)-4-(3,7-di(1H-pyrazol-5-yl)pyrazolo[1,5-a]pyrimidin-5-yl)-3-methylmorpholine

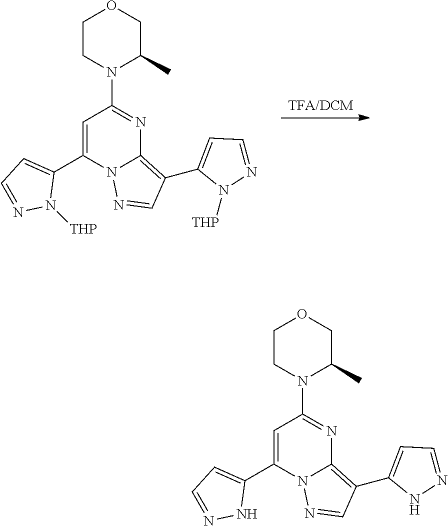

A mixture of (3R)-4-{3,7-bis[1-(oxan-2-yl)-1H-pyrazol-5-yl]pyrazolo[1,5-a]pyrimidin-5-yl}-3-methylmorpholine (38 mg, 0.07 mmol) and TFA (1.0 mL) in DCM (3 mL) was stirred at room temperature for 16 h. LC-MS showed the reaction was complete. The reaction was concentrated under vacuo. The residue was purified by Prep-HPLC (C₁₈, 10-95%, MeOH in H₂O with 0.1% HCOOH) to give the desired product (10 mg, yield: 38%). LC/MS (ESI): m/z 351 [M+H]⁺. ¹H NMR (400 MHz, DMSO) δ 13.62 (s, 1H), 8.40 (s, 1H), 7.99 (s, 1H), 7.68-7.54 (m, 2H), 7.22 (s, 1H), 6.78 (s, 1H), 4.57 (d, J=5.1 Hz, 1H), 4.24 (d, J=12.8 Hz, 1H), 4.02 (dd, J=11.4, 3.0 Hz, 1H), 3.80 (d, J=11.4 Hz, 1H), 3.70 (dd, J=11.4, 2.8 Hz, 1H), 3.57-3.53 (m, 1H), 3.26 (s, 1H), 1.29 (d, J=6.7 Hz, 3H).

### Example 11

### Synthesis of (R)-3-methyl-4-(3-(3-methyl-1H-pyrazol-5-yl)-7-(1-(methylsulfonyl)cyclopropyl)pyrazolo[1,5-a]pyrimidin-5-yl)morpholine

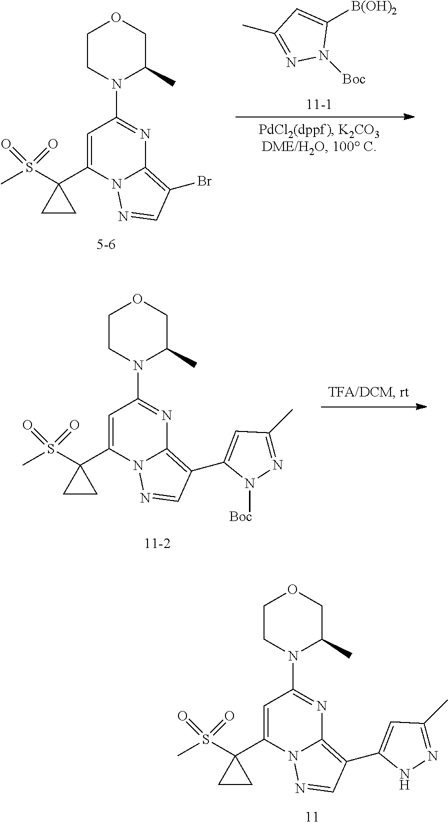

### Step 1. (R)-tert-butyl3-methyl-5-(5-(3-methylmorpholino)-7-(1-(methylsulfonyl)cyclopropyl)pyrazolo[1,5-a]pyrimidin-3-yl)-1H-pyrazole-1-carboxylate

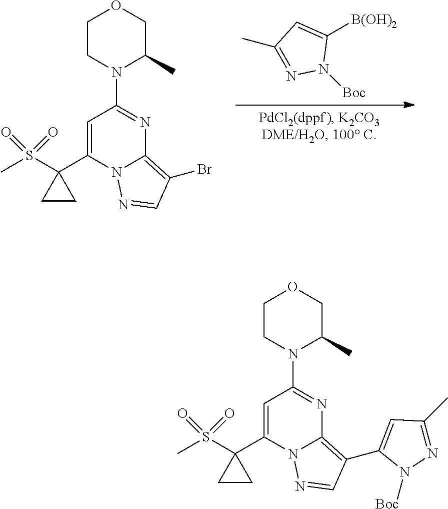

A mixture of (3R)-4-[3-bromo-7-(1-methanesulfonylcyclopropyl)pyrazolo[1,5-a] pyrimidin-5-yl]-3-methylmorpholine (81 mg, 0.19 mmol), {1-[(tert-butoxy)carbonyl]-3-methyl-1H-pyrazol-5-yl}boronic acid (88 mg, 0.38 mmol), PdCl₂(dppf) (14 mg, 0.02 mmol) and K₂CO₃ (67 mg, 0.48 mmol) in co-solvent of DME (3 mL) and H₂O (0.6 mL) was stirred at 100° C. for 16 h under N₂ atmosphere. LC-MS showed the reaction was complete. The mixture was diluted with EA (40 mL), then washed with water and brine, dried over anhydrous Na₂SO₄, filtered and concentrated. The residue was purified by flash chromatography on silica gel (PE:EA=1:1, V/V) to afford the desired product (41 mg, yield: 40%/). LC/MS (ESI): m/z 517 [M+H]⁺.

### Step 2. (R)-3-methyl-4-(3-(3-methyl-1H-pyrazol-5-yl)-7-(1-(methylsulfonyl)cyclopropyl)pyrazolo[1,5-a]pyrimidin-5-yl)morpholine

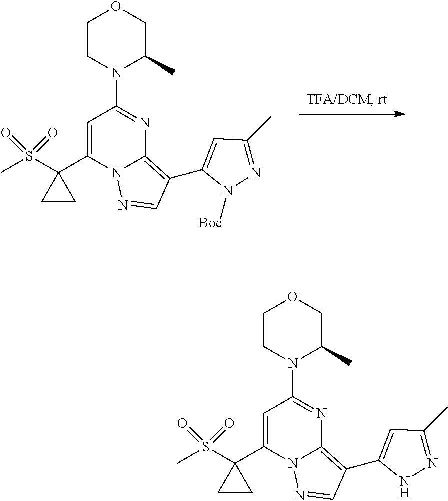

A mixture of (R)-tert-butyl5-[7-(1-methanesulfonylcyclopropyl)-5-[(3R)-3-methyl morpholin-4-yl]pyrazolo[1,5-a]pyrimidin-3-yl]-3-methyl-1H-pyrazole-1-carboxylate (37 mg, 0.07 mmol) and TFA (0.6 mL) in DCM (3 mL) was stirred at room temperature for 1 h. LC-MS showed the reaction was complete. The reaction mixture was concentrated under vacuo. The residue was purified by Prep-HPLC (C₁₈, 10-95%, MeOH in H₂O with 0.1% HCOOH) to give the desired product (10 mg, yield: 33%). LC/MS (ESI): m/z 417 [M+H]⁺. ¹H NMR (400 MHz, DMSO) 12.32 (d, J=53.9 Hz, 1H), 8.27 (d, J=36.6 Hz, 1H), 6.96 (d, J=16.8 Hz, 1H), 6.47 (d, J=40.5 Hz, 1H), 4.56 (dd, J=14.7, 13.4 Hz, 1H), 4.32-4.12 (m, 1H), 4.00 (dd, J=11.5, 3.3 Hz, 1H), 3.79 (d, J=11.5 Hz, 1H), 3.65 (dd, J=11.5, 2.3 Hz, 1H), 3.55-3.45 (m, 1H), 3.27-3.20 (m, 1H), 3.15 (s, 3H), 2.23 (d, J=27.4 Hz, 3H), 1.87 (q, J=5.5 Hz, 2H), 1.63 (q, J=5.7 Hz, 2H), 1.26 (d, J=6.7 Hz, 3H).

### Example 12

### Synthesis of (R)-3-methyl-4-(7-(1-(methylsulfonyl)cyclopropyl)-3-(3-(trifluoromethyl)-1H-pyrazol-5-yl)pyrazolo[1,5-a]pyrimidin-5-yl)morpholine

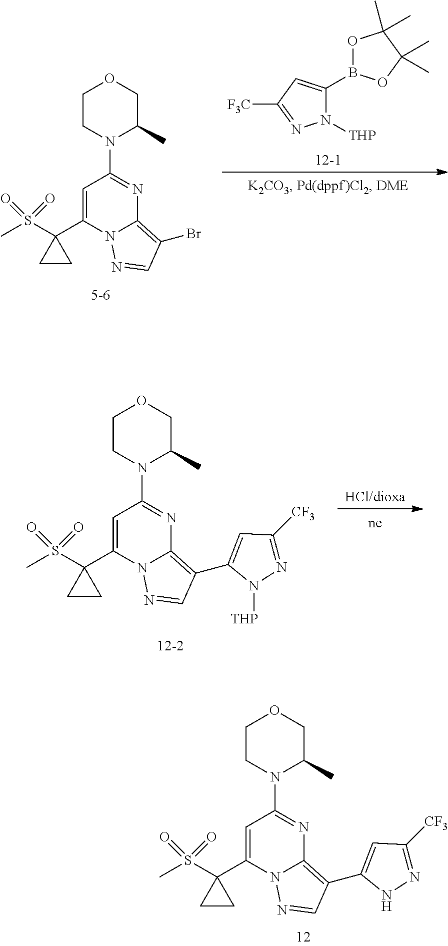

### Step 1. (3R)-3-methyl-4-(7-(1-(methylsulfonyl)cyclopropyl)-3-(1-(tetrahydro-2H-pyran-2-yl)-3-(trifluoromethyl)-1H-pyrazol-5-yl)pyrazolo[1,5-a]pyrimidin-5-yl)morpholine

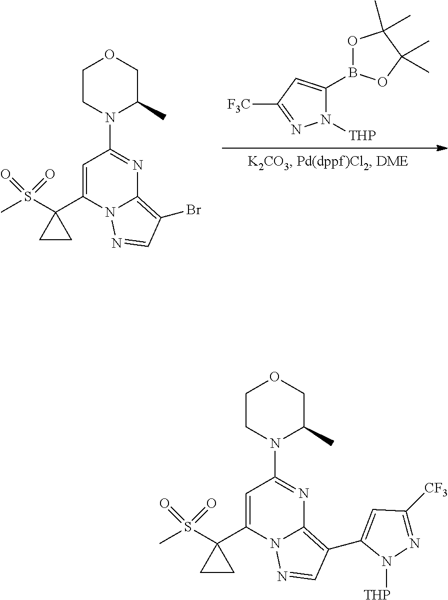

To a solution of (R)-4-(3-bromo-7-(1-(methylsulfonyl)cyclopropyl)pyrazolo[1,5-a]pyrimidin-5-yl)-3-methylmorpholine (100 mg, 0.24 mmol) in DME (5 mL) were added [1-(oxan-2-yl)-3-(trifluoromethyl)-1H-pyrazol-5-yl]boronic acid (127.3 mg, 0.48 mmol), K₂CO₃ (0.36 mL, 0.72 mmol) and Pd(dppf)Cl₂ (17.63 mg, 0.024 mmol). The mixture was stirred at 100° C. overnight under nitrogen atmosphere. LC-MS showed the reaction was complete. The reaction was diluted with EA (40 mL), then washed with water and brine, dried over anhydrous Na₂SO₄, filtered and concentrated. The residue was purified by Prep-TLC (PE:EA=3:1, V/V) to afford the desired product (45 mg, yield: 33%). LC/MS (ESI) m/z: 555 [M+H]⁺.

### Step 2. (R)-3-methyl-4-(7-(1-(methylsulfonyl)cyclopropyl)-3-(3-(trifluoromethyl)-1H-pyrazol-5-yl)pyrazolo[1,5-a]pyrimidin-5-yl)morpholine

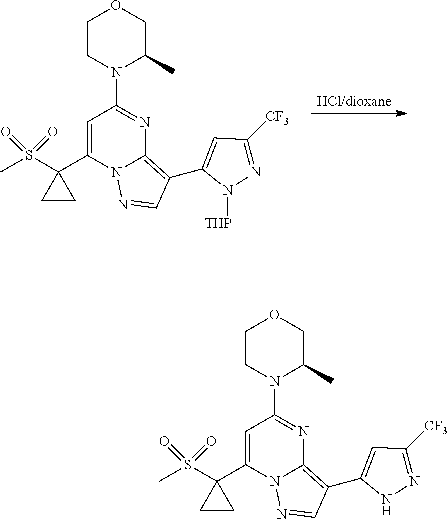

A solution of (3R)-3-methyl-4-(7-(1-(methylsulfonyl)cyclopropyl)-3-(1-(tetrahydro-2H-pyran-2-yl)-3-(trifluoromethyl)-1H-pyrazol-5-yl)pyrazolo[1,5-a]pyrimidin-5-yl)morpholine (45 mg, 0.08 mmol) in DCM (2 mL) was added HCl solution (4M in dioxane, 2 mL). The mixture was stirred at room temperature for 30 min. LC-MS showed the reaction was complete. The reaction mixture was concentrated under vacuo. The residue was purified by Prep-HPLC (C₁₈, 10-95%, MeOH in H₂O with 0.1% HCOOH) to afford the desired product (20 mg, yield: 52%). LC/MS (ESI) m/z: 471 [M+H]⁺. ¹H NMR (400 MHz, DMSO) δ 13.61 (s, 1H), 8.43 (s, 1H), 7.06 (s, 1H), 6.96 (s, 1H), 4.61 (s, 1H), 4.29 (d, J=13.4 Hz, 1H), 4.01 (dd, J=11.3, 2.9 Hz, 1H), 3.80 (d, J=11.4 Hz, 1H), 3.66 (dd, J=11.4, 2.8 Hz, 1H), 3.51 (td, J=11.9, 2.8 Hz, 1H), 3.30-3.22 (m, 1H), 3.16 (s, 3H), 1.89 (dd, J=7.6, 5.4 Hz, 2H), 1.65 (q, J=5.7 Hz, 2H), 1.27 (d, J=6.7 Hz, 3H).

### Example 13

### Synthesis of (R)-4-(3-(3-chloro-1H-pyrazol-5-yl)-7-(1-(methylsulfonyl)cyclopropyl)pyrazolo[1,5-a]pyrimidin-5-yl)-3-methylmorpholine

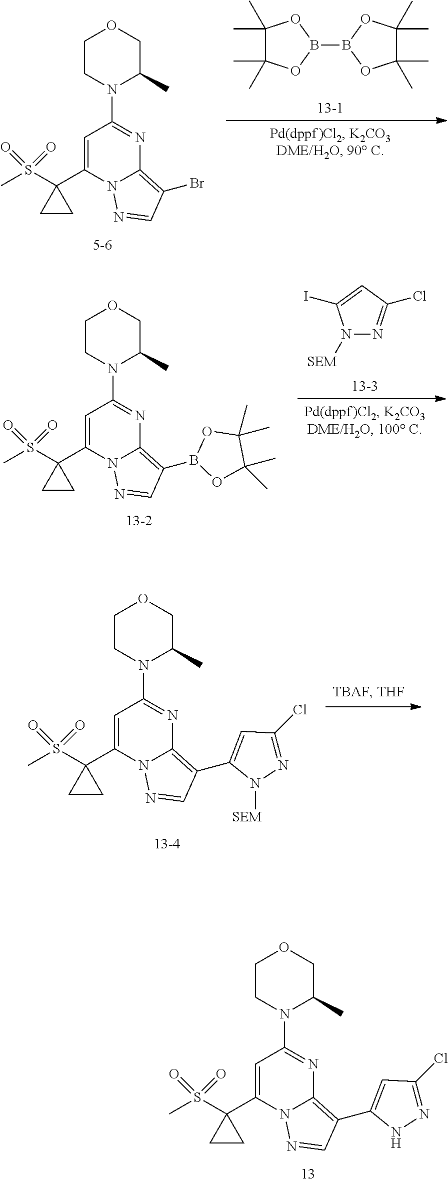

### Step 1. (R)-3-methyl-4-(7-(1-(methylsulfonyl)cyclopropyl)-3-(4,4,5,5-tetramethyl-1,3,2-dioxaborolan-2-yl)pyrazolo[1,5-a]pyrimidin-5-yl)morpholine

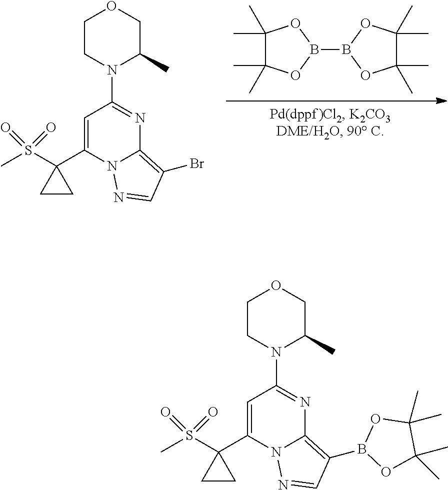

A mixture of (3S)-4-[3-bromo-7-(1-methanesulfonylcyclopropyl)pyrazolo[1,5-a] pyrimidin-5-yl]-3-methylmorpholine (100 mg, 0.25 mmol), 4,4,5,5-tetramethyl-2-(tetramethyl-1,3,2-dioxaborolan-2-yl)-1,3,2-dioxaborolane (250 mg, 1.0 mmol), Pd(dppf)Cl₂ (17.5 mg, 0.025 mmol) and K₂CO₃ (165 mg, 1.2 mmol) in co-solvent of DME (5 mL) and H₂O (0.5 mL) was stirred at 90° C. for 6 h under N₂ atmosphere. LC-MS showed the reaction was complete. The reaction mixture was diluted with EA (40 mL), then washed with water and brine, dried over anhydrous Na₂SO₄, filtered and concentrated. The residue was purified by column chromatography on silica gel (PE:EA=3:1, V/V) to afford the desired product (50 mg, yield: 45%). LC/MS (ESI): m/z 437 [M+H]⁺.

### Step 2. (R)-4-(3-(3-chloro-1-((2-(trimethylsilyl)ethoxy)methyl)-1H-pyrazol-5-yl)-7-(1-(methylsulfonyl)cyclopropyl)pyrazolo[1,5-a]pyrimidin-5-yl)-3-methylmorpholine

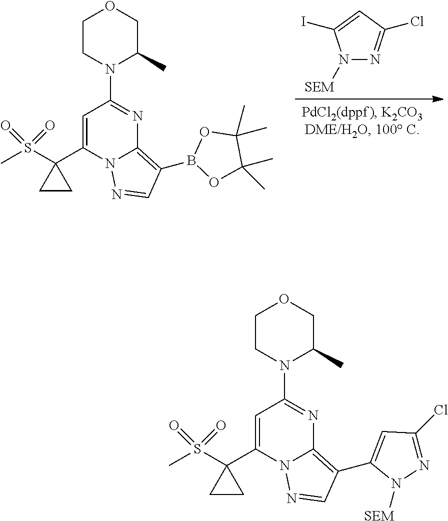

A mixture of (R)-3-methyl-4-(7-(1-(methylsulfonyl)cyclopropyl)-3-(4,4,5,5-tetra methyl-1,3,2-dioxaborolan-2-yl)pyrazolo[1,5-a]pyrimidin-5-yl)morpholine (97 mg, 0.21 mmol), 3-chloro-5-iodo-1-{[2-(trimethylsilyl)ethoxy]methyl}-1H-pyrazole (150 mg, 0.42 mmol), Pd(dppf)Cl₂ (15 mg, 0.02 mmol) and K₂CO₃ (2.0 M in H₂O, 0.26 mL, 0.52 mmol) in DME (4 mL) was stirred at 100° C. for 10 h under N₂ atmosphere. LC-MS showed the reaction was complete. The reaction mixture was diluted with EA (40 mL), then washed with water and brine, dried over anhydrous Na₂SO₄, filtered and concentrated. The residue was purified by column chromatography on silica gel (PE:EA=2:1, V/V) to afford the desired product (63 mg, yield: 52%). LC/MS (ESI): m/z 567 [M+H]⁺.

### Step 3. (R)-4-(3-(3-chloro-1H-pyrazol-5-yl)-7-(1-(methylsulfonyl)cyclopropyl)pyrazolo[1,5-a]pyrimidin-5-yl)-3-methylmorpholine

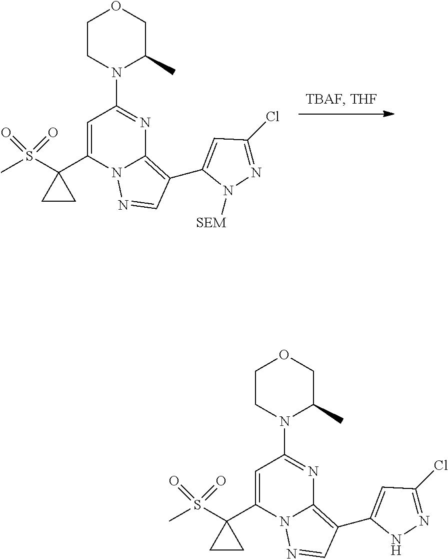

A mixture of (3S)-4-[3-(3-chloro-1-{[2-(trimethylsilyl)ethoxy]methyl}-1H-pyrazol-5-yl)-7-(1-methanesulfonylcyclopropyl)pyrazolo[1,5-a]pyrimidin-5-yl]-3-methylmorpholine (34 mg, 0.06 mmol) in TBAF (1.0 M in THF, 3 mL) was stirred at 70° C. for 2 h. LC-MS showed the reaction was complete. The reaction mixture was diluted with EA (40 mL), then washed with water and brine, dried over anhydrous Na₂SO₄, filtered and concentrated. The residue was purified by column chromatography on silica gel (PE:EA=1:2, V/V) to obtain a crude (45 mg), which was further purified by Pre-HPLC (C₁₈, 10-95%, MeOH in H₂O with 0.1% HCOOH) to give the desired product (20 mg, yield: 76%). LC/MS (ESI): m/z 437 [M+H]. ¹H NMR (400 MHz, DMSO) δ 13.10 (s, 1H), 8.42 (s, 1H), 7.09 (s, 1H), 6.67 (s, 1H), 4.65 (s, 1H), 4.33 (d, J=12.7 Hz, 1H), 4.07 (dd, J=11.5, 3.3 Hz, 1H), 3.85 (d, J=11.5 Hz, 1H), 3.71 (dd, J=11.5, 2.9 Hz, 1H), 3.55 (dt, J=11.8, 6.1 Hz, 1H), 3.33-3.26 (m, 1H), 3.21 (s, 3H), 1.94 (dd, J=7.7, 5.4 Hz, 2H), 1.70 (q, J=5.7 Hz, 2H), 1.32 (d, J=6.7 Hz, 3H).

### Example 14

### Synthesis of (R)-3-methyl-4-(3-(4-methyl-1H-pyrazol-5-yl)-7-(1-(methylsulfonyl)cyclopropyl)pyrazolo[1,5-a]pyrimidin-5-yl)morpholine

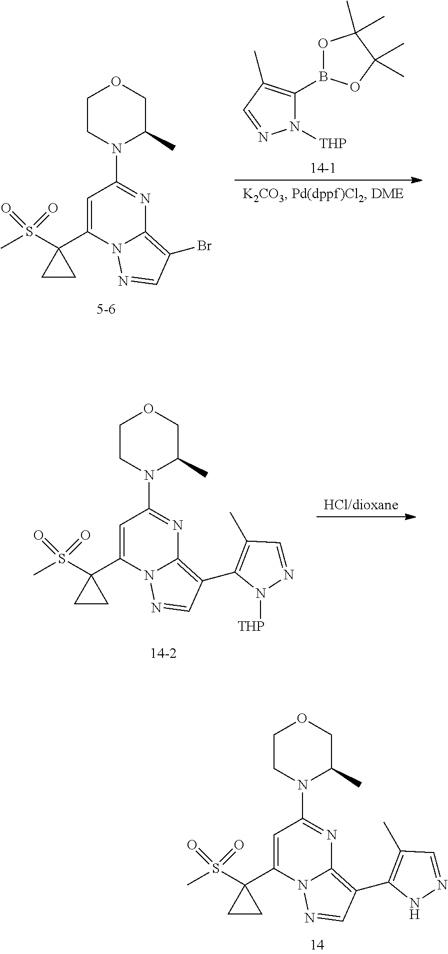

### Step 1. Ethyl (3R)-3-methyl-4-(3-(4-methyl-1-(tetrahydro-2H-pyran-2-yl)-1H-pyrazol-5-yl)-7-(1-(methylsulfonyl)cyclopropyl)pyrazolo[1,5-a]pyrimidin-5-yl)morpholine

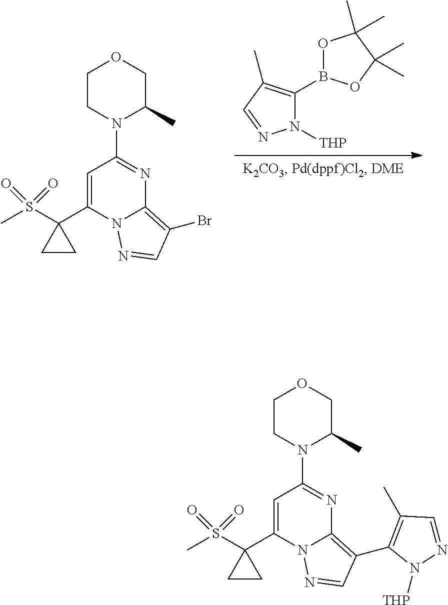

To a solution of (R)-4-(3-bromo-7-(1-(methylsulfonyl)cyclopropyl)pyrazolo[1,5-a] pyrimidin-5-yl)-3-methylmorpholine (100 mg, 0.24 mmol) in DME (10 mL) were added 4-methyl-1-(oxan-2-yl)-5-(tetramethyl-1,3,2-dioxaborolan-2-yl)-1H-pyrazole (140.7 mg, 0.48 mmol), K₂CO₃ (2M in H₂O, 0.36 mL, 0.72 mmol) and Pd(dppf)Cl₂ (17.6 mg, 0.02 mmol). The mixture was stirred at 100° C. overnight under nitrogen atmosphere. LC-MS showed the reaction was complete. The reaction mixture was diluted with EA (40 mL), then washed with water and brine, dried over anhydrous Na₂SO₄, filtered and concentrated.

The residue was purified by column chromatography on silica gel (PE:EA=2:1, V/V) to afford the desired product (50 mg, yield: 41%). LC/MS (ESI) m/z: 501 [M+H]⁺.

### Step 2. (R)-3-methyl-4-(3-(4-methyl-1H-pyrazol-5-yl)-7-(1-(methylsulfonyl)cyclopropyl)pyrazolo[1,5-a]pyrimidin-5-yl)morpholine

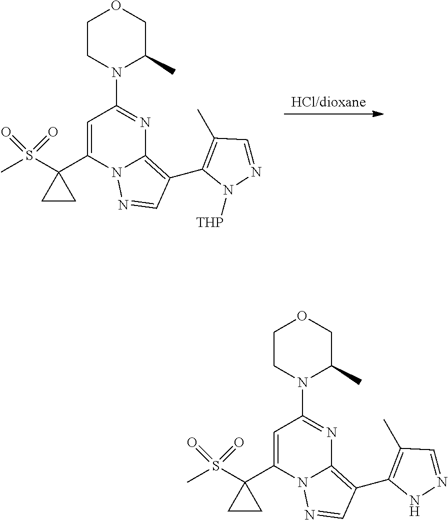

A mixture of ethyl (3R)-3-methyl-4-(3-(4-methyl-1-(tetrahydro-2H-pyran-2-yl)-1H-pyrazol-5-yl)-7-(1-(methylsulfonyl)cyclopropyl)pyrazolo[1,5-a]pyrimidin-5-yl)morpholine (50 mg, 0.1 mmol) in DCM (1 mL) was added HCl solution (4M in dioxane, 1 mL). The mixture was stirred at room temperature for 30 min. LC-MS showed the reaction was complete. The reaction mixture was concentrated under vacuo. The residue was purified by Prep-HPLC (C₁₈, 10-95%, MeOH in H₂O with 0.1% HCOOH) to afford the desired product (15 mg, yield: 36%). LC-MS (ESI) m/z: 417 [M+H]⁺. ¹H NMR (400 MHz, DMSO) δ 12.34 (s, 1H), 8.15 (s, 1H), 7.33 (s, 1H), 6.99 (s, 1H), 4.57 (s, 1H), 4.20 (s, 1H), 4.01-3.90 (m, 1H), 3.75 (d, J=11.3 Hz, 1H), 3.62 (dd, J=11.6, 2.9 Hz, 1H), 3.46 (dt, J=11.8, 5.9 Hz, 1H), 3.22 (dd, J=13.1, 3.4 Hz, 1H), 3.18 (s, 3H), 2.17 (s, 3H), 1.89 (dd, J=7.7, 5.4 Hz, 2H), 1.65 (q, J=5.7 Hz, 2H), 1.22 (d, J=6.7 Hz, 3H).

### Example 15

### Synthesis of (3R)-3-methyl-4-[7-(1-methyl-1H-pyrazol-4-yl)-3-(1H-pyrazol-5-yl)pyrazolo[1,5-a]pyrimidin-5-yl]morpholine

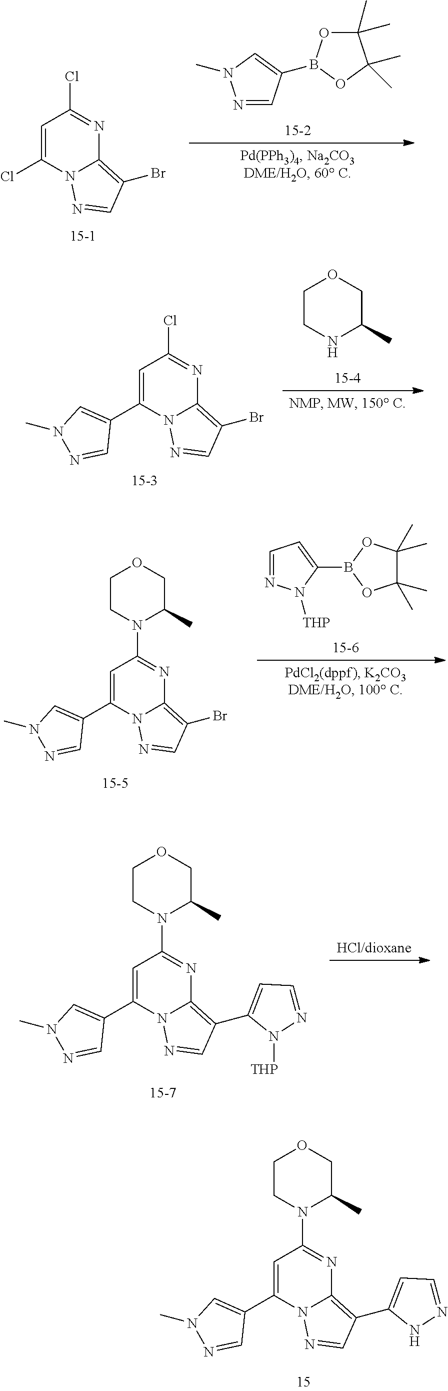

### Step 1. 4-{3-bromo-5-chloropyrazolo[1,5-a]pyrimidin-7-yl}-1-methyl-1H-pyrazole

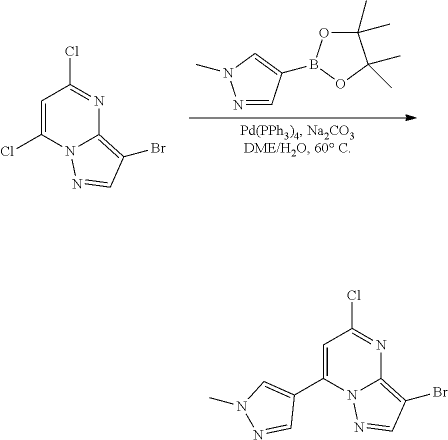

A mixture of 3-bromo-5,7-dichloropyrazolo[1,5-a]pyrimidine (400 mg, 1.50 mmol), 1-methyl-4-(tetramethyl-1,3,2-dioxaborolan-2-yl)-1H-pyrazole (311.8 mg, 1.50 mmol), Pd(PPh₃)₄ (173.2 mg, 0.15 mmol) and Na₂CO₃ (2M in H₂O, 1.5 mL, 2.99 mmol) in DME (15 mL) was stirred at 60° C. for 3 h under N₂ atmosphere. LC-MS showed the reaction was complete. The reaction mixture was diluted with EA (40 mL), then washed with water and brine, dried over anhydrous Na₂SO₄, filtered and concentrated. The residue was purified by column chromatography on silica gel (PE:EA=10:1, V/V) to afford the desired product (400 mg, yield: 85%). LC/MS (ESI): m/z 312/314 [M+H]⁺.

### Step 2. (3R)-4-[3-bromo-7-(1-methyl-1H-pyrazol-4-yl)pyrazolo[1,5-a]pyrimidin-5-yl]-3-methylmorpholine

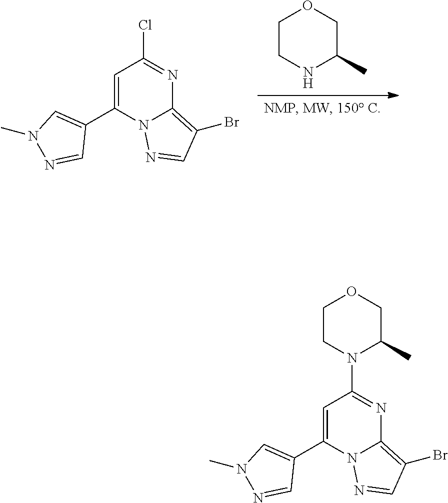

A mixture of 4-{3-bromo-5-chloropyrazolo[1,5-a]pyrimidin-7-yl}-1-methyl-1H-pyrazole (200 mg, 0.64 mmol) and (3R)-3-methylmorpholine (194.2 mg, 1.92 mmol) in NMP (3 mL) was stirred at 150° C. for 1 h under microwave irradiation.

LC-MS showed the reaction was complete. The reaction mixture was diluted with EA (40 mL), then washed with water and brine, dried over anhydrous Na₂SO₄, filtered and concentrated. The residue was purified by column chromatography on silica gel (PE:EA=3:1, V/V) to afford the desired product (200 mg, yield: 82%). LC/MS (ESI): m/z 377/379 [M+H]⁺.

### Step 3. (3R)-3-methyl-4-[7-(1-methyl-1H-pyrazol-4-yl)-3-[1-(oxan-2-yl)-1H-pyrazol-5-yl]pyrazolo[1,5-a]pyrimidin-5-yl]morpholine

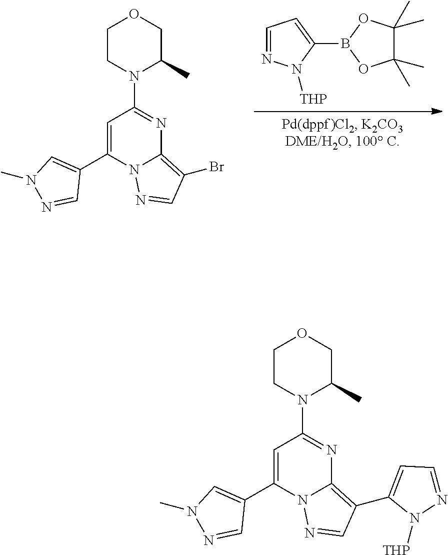

A mixture of (3R)-4-[3-bromo-7-(1-methyl-1H-pyrazol-4-yl)pyrazolo[1,5-a]pyrimidin-5-yl]-3-methylmorpholine (200 mg, 0.53 mmol), 1-(oxan-2-yl)-5-(tetramethyl-1,3,2-dioxaborolan-2-yl)-1H-pyrazole (221.2 mg, 0.80 mmol), K₂CO₃ (183.2 mg, 1.33 mmol) and Pd(dppf)Cl₂ (38.8 mg, 0.05 mmol) in co-solvent of DME (5 mL) and H₂O (1 mL) was stirred at 100° C. for 5 h under N₂ atmosphere. LC-MS showed the reaction was complete. The reaction mixture was diluted with EA (40 mL), then washed with water and brine, dried over anhydrous Na₂SO₄, filtered and concentrated. The residue was purified by column chromatography on silica gel (PE:EA=1:1, V/V) to afford the desired product (120 mg, yield: 50%). LC/MS (ESI): m/z 449 [M+H]⁺.

### Step 4. (3R)-3-methyl-4-[7-(1-methyl-1H-pyrazol-4-yl)-3-(1H-pyrazol-5-yl)pyrazolo[1,5-a]pyrimidin-5-yl]morpholine

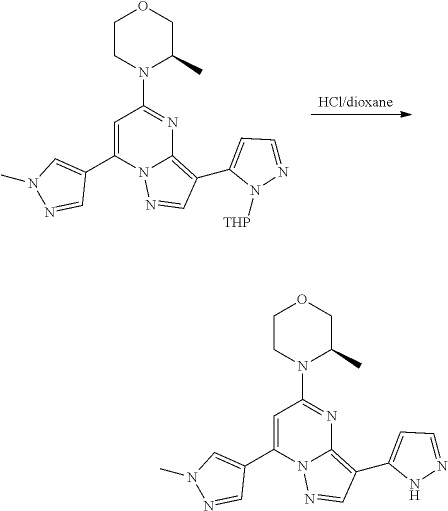

To a solution of (3R)-3-methyl-4-[7-(1-methyl-1H-pyrazol-4-yl)-3-[1-(oxan-2-yl)-1H-pyrazol-5-yl]pyrazolo[1,5-a]pyrimidin-5-yl]morpholine (100 mg, 0.22 mmol) in DCM (4 mL) was added HCl solution (4M in dioxane, 1.5 mL). The mixture was stirred at room temperature for 0.5 h. LC-MS showed the reaction was complete. The reaction mixture was concentrated under reduced pressure. The residue was purified by Prep-HPLC (C₁₈, 10-95%, MeOH in H₂O with 0.1% HCOOH) to give the desired product (40 mg, yield: 49%). LC/MS (ESI): m/z 365 [M+H]⁺. ¹H NMR (400 MHz, DMSO) δ 12.74 (s, 1H), 8.97 (s, 1H), 8.58 (s, 1H), 8.38 (s, 1H), 7.59 (s, 1H), 7.08 (s, 1H), 6.76 (s, 1H), 4.66 (d, J=5.3 Hz, 1H), 4.28 (d, J=13.7 Hz, 1H), 4.02 (dd, J=11.4, 3.3 Hz, 1H), 3.81 (d, J=11.3 Hz, 1H), 3.69 (dd, J=11.4, 2.8 Hz, 1H), 3.53 (td, J=12.0, 2.9 Hz, 1H), 3.25 (dd, J=12.9, 3.5 Hz, 1H), 1.27 (d, J=6.7 Hz, 3H).

### Example 16

### Synthesis of (R)-3-methyl-4-(7-(4-(methylsulfonyl)phenyl)-3-(1H-pyrazol-5-yl)pyrazolo[1,5-a]pyrimidin-5-yl)morpholine

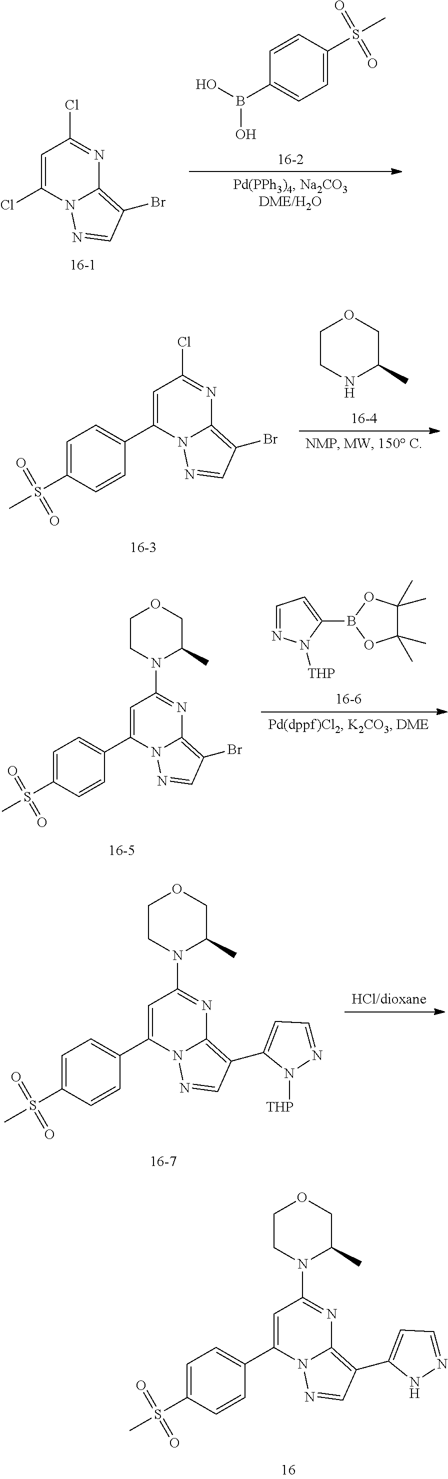

### Step 1. 3-bromo-5-chloro-7-(4-methanesulfonylphenyl)pyrazolo[1,5-a]pyrimidine

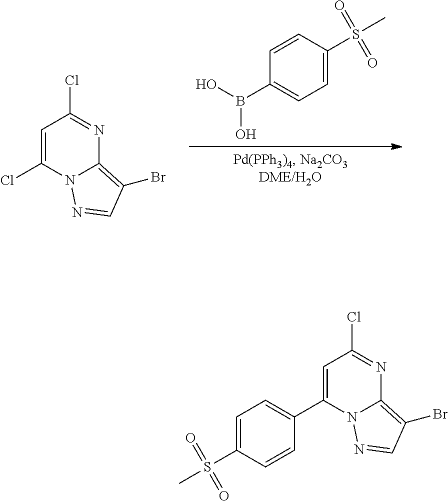 [SMILES: Clc1cc(Cl)n2ncc(Br)c2n1 ; CS(=O)(=O)c1ccc(B(O)O)cc1 ; "Pd(PPh3)4, Na2CO3DME/H2O" ; CS(=O)(=O)c1ccc(-c2cc(Cl)nc3c(Br)cnn23)cc1]

A suspension of 3-bromo-5,7-dichloropyrazolo[1,5-a]pyrimidine (400 mg, 1.50 mmol), (4-methanesulfonylphenyl)boronic acid (300 mg, 1.50 mmol), Pd(PPh₃)₄ (173.2 mg, 0.15 mmol) and Na₂CO₃₁ (2M in H₂O, 1.50 mL, 2.99 mmol) in DME (15 mL) was stirred at 60° C. for 3 h under N₂ atmosphere. LC-MS showed the reaction was complete. The reaction mixture was diluted with EA (40 mL), then washed with water and brine, dried over anhydrous Na₂SO₄, filtered and concentrated. The residue was purified by column chromatography on silica gel (PE:EA=10:1, V/V) to afford the desired product (350 mg, yield: 60%/). LC/MS (ESI): m/z 386/388 [M+H]⁺.

### Step 2. (3R)-4-[3-bromo-7-(4-methanesulfonylphenyl)pyrazolo[1,5-a]pyrimidin-5-yl]-3-methylmorpholine

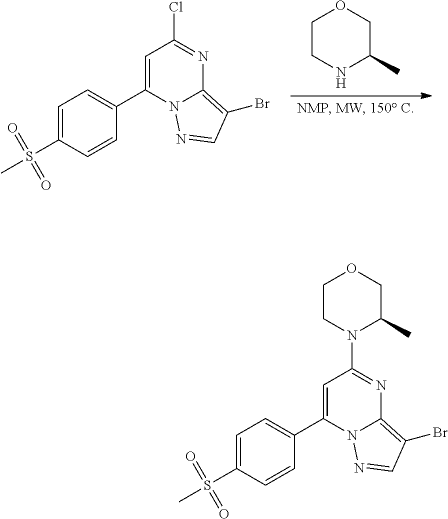

A mixture of 3-bromo-5-chloro-7-(4-methanesulfonylphenyl)pyrazolo[1,5a]pyrimidine (200 mg, 0.52 mmol) and (3R)-3-methylmorpholine (157 mg, 1.55 mmol) in NMP (3 mL) was stirred at 150° C. for 1 h under microwave irradiation. LC-MS showed the reaction was complete. The reaction mixture was diluted with EA (40 mL), then washed with water and brine, dried over anhydrous Na₂SO₄, filtered and concentrated. The residue was purified by column chromatography on silica gel (PE:EA=3:1, V/V) to afford the desired product (180 mg, yield: 77%). LC/MS (ESI): m/z 451/453 [M+H]⁺.

### Step 3. (3R)-3-methyl-4-(7-(4-(methylsulfonyl)phenyl)-3-(1-(tetrahydro-2H-pyran-2-yl)-1H-pyrazol-5-yl)pyrazolo[1,5-a]pyrimidin-5-yl)morpholine

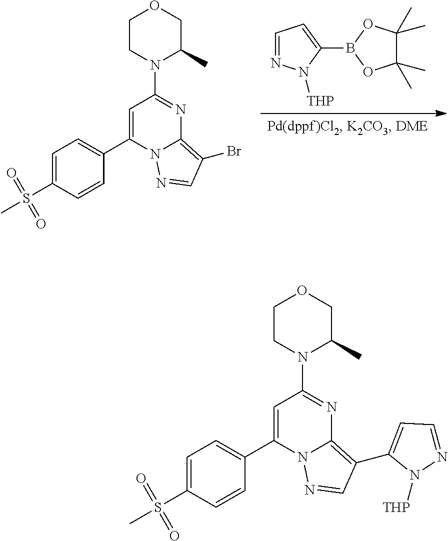

To a solution of (R)-4-(3-bromo-7-(4-(methylsulfonyl)phenyl)pyrazolo[1,5-a]pyrimidin-5-yl)-3-methylmorpholine (60 mg, 0.15 mmol) and 1-(oxan-2-yl)-5-(tetra methyl-1,3,2-dioxaborolan-2-yl)-1H-pyrazole (86.3 mg, 0.31 mmol) in DME (5 mL) were added K₂CO₃ (54 mg, 0.39 mmol) and Pd(PPh₃)₄ (18 mg, 0.02 mmol). The mixture was stirred at 90° C. for 3 h under N₂ atmosphere. LC-MS showed the reaction was complete. The reaction mixture was diluted with EA (40 mL), then washed with water and brine, dried over anhydrous Na₂SO₄, filtered and concentrated. The residue was purified by column chromatography on silica gel (PE:EA=1:1, V/V) to afford the desired product (25 mg, yield: 35%). LC/MS (ESI) m/z: 523 [M+H]⁺.

### Step 4. (R)-3-methyl-4-(7-(4-(methylsulfonyl)phenyl)-3-(1H-pyrazol-5-yl)pyrazolo[1,5-a]pyrimidin-5-yl)morpholine

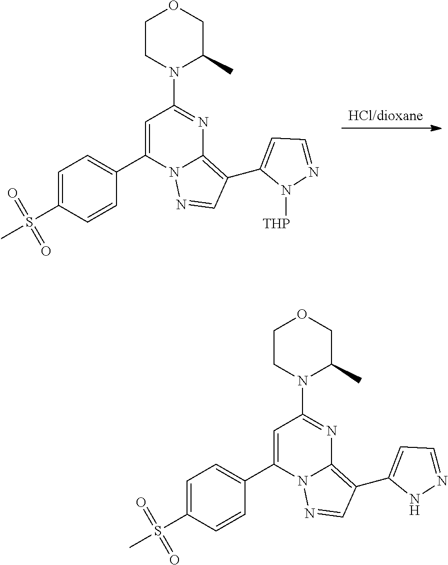

A mixture of (3R)-3-methyl-4-(7-(4-(methylsulfonyl)phenyl)-3-(1-(tetrahydro-2H-pyran-2-yl)-1H-pyrazol-5-yl)pyrazolo[1,5-a]pyrimidin-5-yl)morpholine (40 mg, 0.08 mmol) in DCM (2 mL) was added HCl (4M in dioxane, 2 mL). The mixture was stirred at room temperature for 30 min. LC-MS showed the reaction was complete. The reaction mixture was concentrated under vacuo. The residue was purified by Pre-HPLC (C₁₈, 10-95%, MeOH in H₂O with 0.1% HCOOH) to afford the desired product (20 mg, yield: 59%). LC/MS (ESI) m/z: 439 [M+H]⁺. ¹H NMR (400 MHz, CDCl₃) δ 8.18 (s, 1H), 8.16-8.09 (m, 4H), 7.63 (d, J=1.7 Hz, 1H), 6.52 (s, 1H), 6.43 (s, 1H), 4.46 (d, J=4.5 Hz, 1H), 4.18-4.06 (m, 2H), 3.89 (d, J=11.5 Hz, 1H), 3.81 (dd, J=11.6, 2.9 Hz, 1H), 3.66 (td, J=12.0, 3.1 Hz, 1H), 3.47 (td, J=12.8, 3.8 Hz, 1H), 3.12 (s, 3H), 1.44 (d, J=6.8 Hz, 3H).

### Example 17

### Synthesis of (R)-3-methyl-4-(7-(4-(methylsulfonyl)piperazin-1-yl)-3-(1H-pyrazol-5-yl)pyrazolo[1,5-a]pyrimidin-5-yl)morpholine

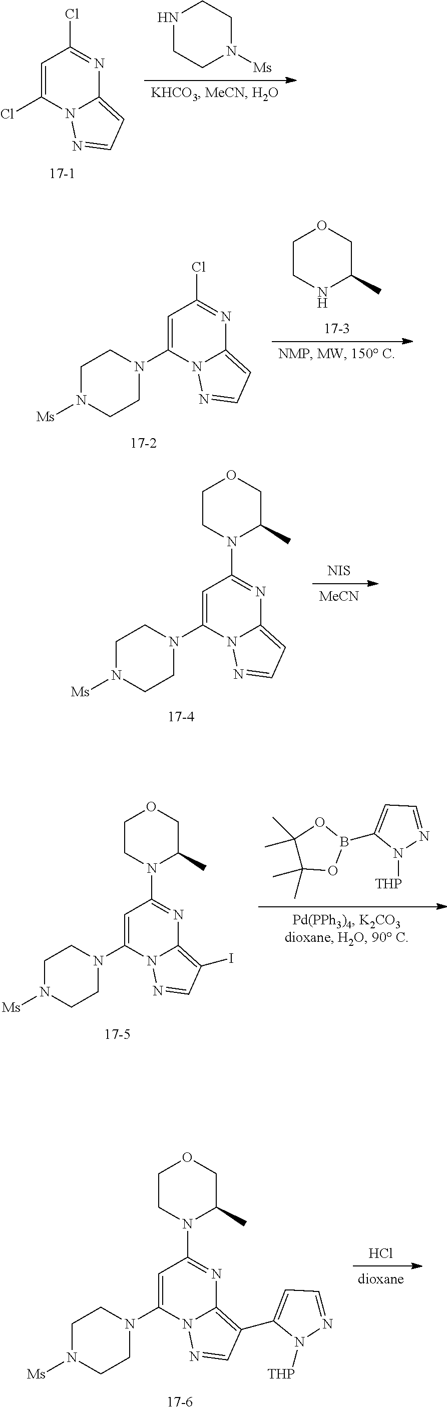

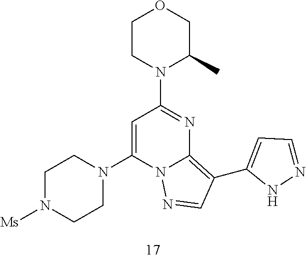 [SMILES: C[C@@H]1COCCN1c1cc(N2CCN(S(C)(=O)=O)CC2)n2ncc(-c3ccn[nH]3)c2n1]

### Step 1. 5-chloro-7-(4-(methylsulfonyl)piperazin-1-yl)pyrazolo[1,5-a]pyrimidine

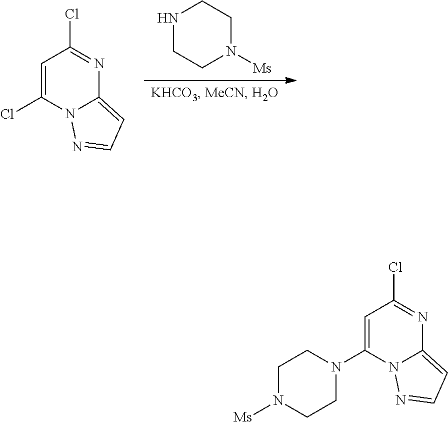

To a solution of 5,7-dichloropyrazolo[1,5-a]pyrimidine (940 mg, 5.0 mmol) and 1-methanesulfonylpiperazine (821 mg, 5.0 mmol) in CH₃CN (12 mL) and H₂O (12 mL) was added KHCO₃ (1.0 g, 10.0 mmol). The mixture was stirred at room temperature overnight. LC-MS showed the reaction was complete. The reaction mixture was diluted with EA (40 mL), then washed with water and brine, dried over anhydrous Na₂SO₄, filtered and concentrated. The residue was purified by column chromatography on silica gel (PE:EA=10:1, V/V) to afford the desired product (1.45 g, yield: 92%). LC/MS (ESI): m/z 316 [M+H]⁺. ¹H NMR (400 MHz, CDCl₃) δ 8.04 (d, J=2.3 Hz, 1H), 6.54 (d, J=2.3 Hz, 1H), 6.13 (s, 1H), 3.89-3.85 (m, 4H), 3.53-3.49 (m, 4H), 2.86 (s, 3H).

### Step 2. (R)-3-methyl-4-(7-(4-(methylsulfonyl)piperazin-1-yl)pyrazolo[1,5-a]pyrimidin-5-yl)morpholine

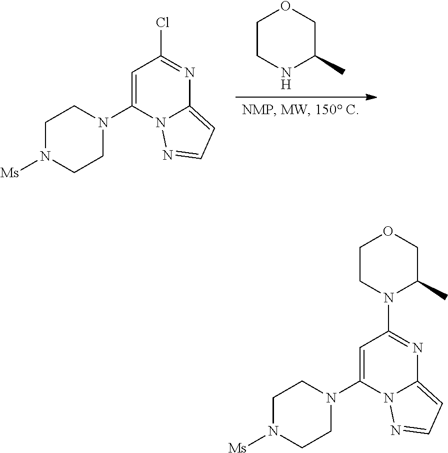

To a solution of 1-{5-chloropyrazolo[1,5-a]pyrimidin-7-yl}-4-methanesulfonylpiperazine (205 mg, 0.65 mmol) and (3R)-3-methylmorpholine (197 mg, 1.95 mmol) in NMP (3 mL) was added KHCO₃ (292 mg, 2.92 mmol). The mixture was stirred at 150° C. for 1 h under microwave irradiation. LC-MS showed the reaction was complete. The reaction mixture was diluted with EA (40 mL), then washed with water and brine, dried over anhydrous Na₂SO₄, filtered and concentrated.

The residue was purified by column chromatography on silica gel (PE:EA=6:1, V/V) to afford the desired product (150 mg, yield: 61%). LC/MS (ESI): m/z 381 [M+H]⁺. ¹H NMR (400 MHz, CDCl₃) δ 7.86 (d, J=2.2 Hz, 1H), 6.12 (d, J=2.1 Hz, 1H), 5.55 (s, 1H), 4.32 (d, J=4.8 Hz, 1H), 4.04-3.96 (m, 2H), 3.78 (dd, J=18.3, 7.2 Hz, 2H), 3.72-3.65 (m, 4H), 3.57 (dd, J=11.8, 3.1 Hz, 1H), 3.51 (t, J=4.9 Hz, 4H), 3.30 (t, J=4.6 Hz, 1H), 2.85 (s, 3H), 1.31 (d, J=6.8 Hz, 3H).

### Step 3. (R)-4-(3-iodo-7-(4-(methylsulfonyl)piperazin-1-yl)pyrazolo[1,5-a]pyrimidin-5-yl)-3-methylmorpholine

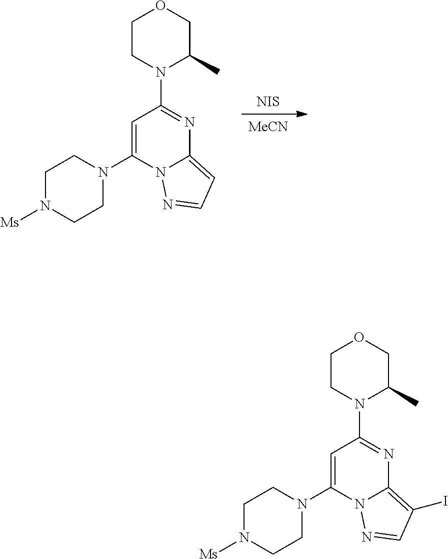

To a solution of (3R)-4-[7-(4-methanesulfonylpiperazin-1-yl)pyrazolo[1,5-a]pyrimidin-5-yl]-3-methylmorpholine (140 mg, 0.37 mmol) in CH₃CN (10 mL) was added NIS (91 mg, 0.41 mmol). The mixture was stirred at room temperature for 30 min. LC-MS showed the reaction was complete. The reaction mixture was diluted with EA (40 mL), then washed with saturated Na₂S₂O₃ solution and brine, dried over anhydrous Na₂SO₄, filtered and concentrated. The residue was purified by column chromatography on silica gel (PE:EA=6:1, V/V) to afford the desired product (160 mg, yield: 86%). LC/MS (ESI): m/z 507 [M+H]⁺. ¹H NMR (400 MHz, CDCl₃) δ 7.83 (s, 1H), 5.55 (s, 1H), 4.35 (d, J=6.8 Hz, 1H), 4.15 (d, J=11.6 Hz, 1H), 4.04 (dd, J=11.4, 3.7 Hz, 1H), 3.82 (d, J=11.4 Hz, 1H), 3.76 (d, J=3.0 Hz, 1H), 3.63 (dd, J=8.0, 3.9 Hz, 4H), 3.58 (dd, J=11.7, 3.0 Hz, 1H), 3.50 (t, J=4.8 Hz, 4H), 3.32 (dd, J=12.9, 9.0 Hz, 1H), 2.85 (s, 3H), 1.34 (d, J=6.8 Hz, 3H).

### Step 4. (3R)-3-methyl-4-(7-(4-(methylsulfonyl)piperazin-1-yl)-3-(1-(tetrahydro-2H-pyran-2-yl)-1H-pyrazol-5-yl)pyrazolo[1,5-a]pyrimidin-5-yl)morpholine

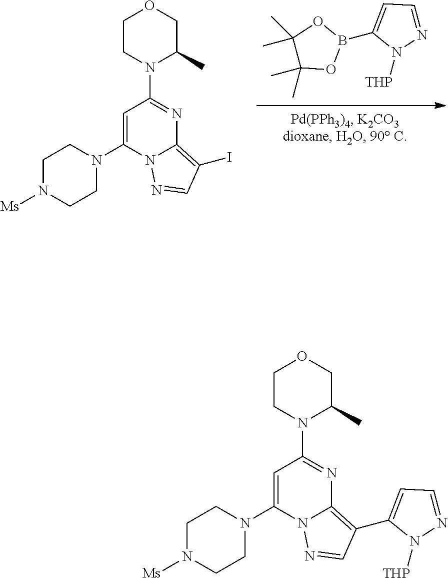

A mixture of (3R)-4-[3-iodo-7-(4-methanesulfonylpiperazin-1-yl)pyrazolo[1,5-a]pyrimidin-5-yl]-3-methylmorpholine (163 mg, 0.32 mmol), 1-(oxan-2-yl)-3-(tetramethyl-1,3,2-dioxaborolan-2-yl)-1H-pyrazole (134 mg, 0.48 mmol), Pd(dppf)Cl₂ (24 mg, 0.03 mmol) and K₂CO₃ (111 mg, 0.81 mmol) in co-solvent of dioxane (10 mL) and H₂O (2 mL) was stirred at 100° C. overnight under N₂ atmosphere. LC-MS showed the reaction was complete. The reaction mixture was diluted with EA (40 mL), then washed with water and brine, dried over anhydrous Na₂SO₄, filtered and concentrated. The residue was purified by column chromatography on silica gel (PE:EA=3:1, V/V) to afford the desired product (120 mg, yield: 70%). LC/MS (ESI): m/z 531 [M+H]⁺.

### Step 5. (R)-3-methyl-4-(7-(4-(methylsulfonyl)piperazin-1-yl)-3-(1H-pyrazol-5-yl)pyrazolo[1,5-a]pyrimidin-5-yl)morpholine

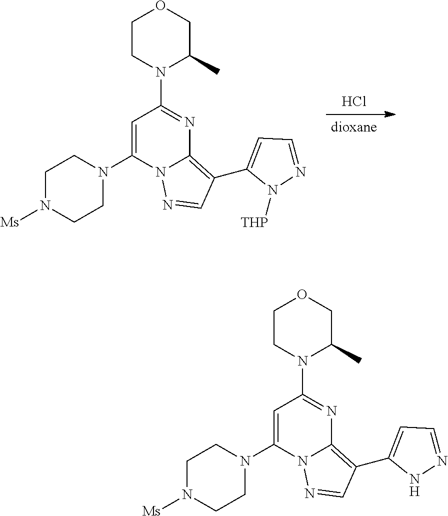

A mixture of (3R)-3-methyl-4-(7-(4-(methylsulfonyl)piperazin-1-yl)-3-(1-(tetrahydro-2H-pyran-2-yl)-1H-pyrazol-5-yl)pyrazolo[1,5-a]pyrimidin-5-yl)morpholine (120 mg, 0.23 mmol) in DCM (4 mL) was added HCl solution (4 M in dioxane, 4 mL). The mixture was stirred at room temperature for 2 h. LC-MS showed the reaction was complete. The reaction mixture was concentrated under vacuo. The residue was purified by Prep-HPLC (C18, 20-95%, acetonitrile in H₂O with 0.1% HCOOH) to give the desired product (25.2 mg, yield: 25%). LC/MS (ESI): m/z 447 [M+H]⁺. ¹H NMR (400 MHz, DMSO) δ 12.81-12.47 (m, 1H), 8.43-8.05 (m, 1H), 7.81-7.36 (m, 1H), 6.89-6.56 (m, 1H), 5.91 (s, 1H), 4.56 (s, 1H), 4.15 (s, 1H), 3.99 (dd, J=11.3, 3.4 Hz, 1H), 3.79-3.62 (m, 6H), 3.52-3.45 (m, 1H), 3.36-3.33 (m, 4H), 3.21 (td, J=12.8, 3.6 Hz, 1H), 2.97 (s, 3H), 1.24 (d, J=6.7 Hz, 3H).

### Example 18

### Synthesis of (R)-3-methyl-4-(7-(1-methyl-1H-pyrazol-5-yl)-3-(3-methyl-1H-pyrazol-5-yl)pyrazolo[1,5-a]pyrimidin-5-yl)morpholine

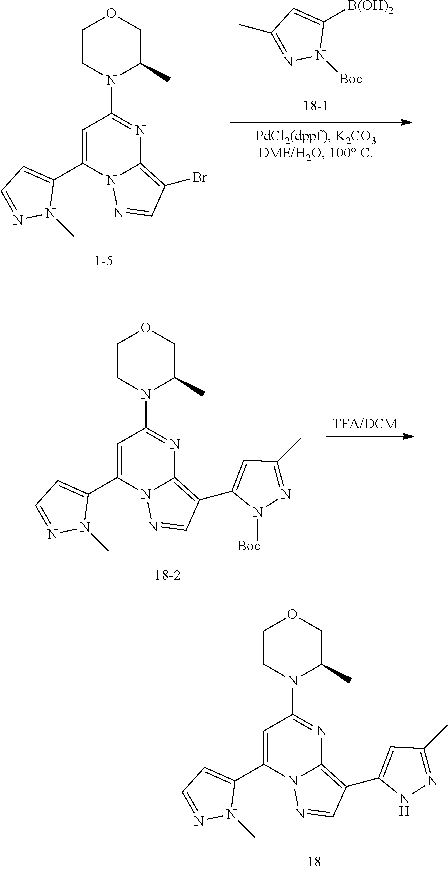 [SMILES: C[C@@H]1COCCN1c1cc(-c2ccnn2C)n2ncc(Br)c2n1 ; Cc1cc(B(O)O)n(C(=O)OC(C)(C)C)n1 ; "PdCl2(dppf), K2CO3DME/H2O, 100° C." ; Cc1cc(-c2cnn3c(-c4ccnn4C)cc(N4CCOC[C@H]4C)nc23)n(C(=O)OC(C)(C)C)n1 ; "TFA/DCM" ; Cc1cc(-c2cnn3c(-c4ccnn4C)cc(N4CCOC[C@H]4C)nc23)[nH]n1]

### Step 1. tert-butyl(R)-3-methyl-5-(7-(1-methyl-1H-pyrazol-5-yl)-5-(3-methylmorpholino)pyrazolo[1,5-a]pyrimidin-3-yl)-1H-pyrazole-1-carboxylate

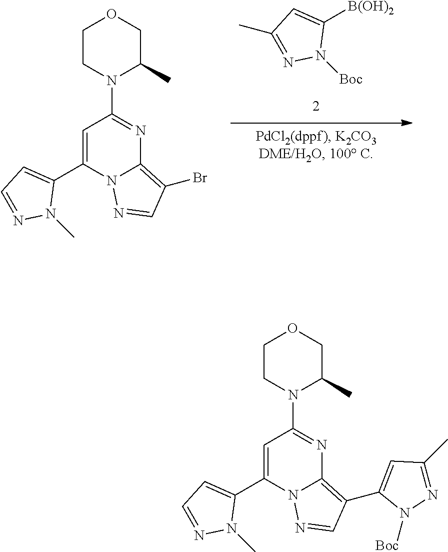 [SMILES: C[C@@H]1COCCN1c1cc(-c2ccnn2C)n2ncc(Br)c2n1 ; Cc1cc(B(O)O)n(C(=O)OC(C)(C)C)n1 ; "PdCl2(dppf), K2CO3DME/H2O, 100° C." ; Cc1cc(-c2cnn3c(-c4ccnn4C)cc(N4CCOC[C@H]4C)nc23)n(C(=O)OC(C)(C)C)n1]

A mixture of (3R)-4-[3-bromo-7-(1-methyl-1H-pyrazol-5-yl)pyrazolo[1,5-a]pyrimidin-5-yl]-3-methylmorpholine (102 mg, 0.27 mmol), {1-[(tert-butoxy)carbonyl]-3-methyl-1H-pyrazol-5-yl}boronic acid (79 mg, 0.35 mmol), Pd(dppf)Cl₂ (20 mg, 0.027 mmol) and K₂CO₃ (93 mg, 0.68 mmol) in co-solvent of dioxane (5 mL) and H₂O (1 mL) was stirred at 90° C. overnight under N₂ atmosphere. LC-MS showed the reaction was complete. The reaction mixture was diluted with EA (40 mL), then washed with water and brine, dried over anhydrous Na₂SO₄, filtered and concentrated. The residue was purified by column chromatography on silica gel (PE:EA=10:1, V/V) to afford the desired product (102 mg, yield: 78%). LC/MS (ESI): m/z 479 [M+H]⁺.

### Step 2. (R)-3-methyl-4-(7-(1-methyl-1H-pyrazol-5-yl)-3-(3-methyl-1H-pyrazol-5-yl)pyrazolo[1,5-a]pyrimidin-5-yl)morpholine

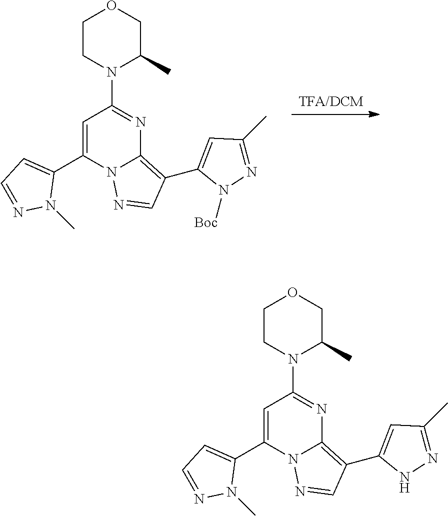 [SMILES: Cc1cc(-c2cnn3c(-c4ccnn4C)cc(N4CCOC[C@H]4C)nc23)n(C(=O)OC(C)(C)C)n1 ; "TFA/DCM" ; Cc1cc(-c2cnn3c(-c4ccnn4C)cc(N4CCOC[C@H]4C)nc23)[nH]n1]

A mixture of tert-butyl3-methyl-5-[7-(1-methyl-1H-pyrazol-5-yl)-5-[(3R)-3-methylmorpholin-4-yl]pyrazolo[1,5-a]pyrimidin-3-yl]-1H-pyrazole-1-carboxylate (102 mg, 0.21 mmol) in DCM (3 mL) was added HCl solution (4 M in dioxane, 3 mL). The mixture was stirred at room temperature for 2 h. LC-MS showed the reaction was complete. The reaction mixture was concentrated under vacuo. The residue was purified by Prep-HPLC (C18, 20-95%, acetonitrile in H₂O with 0.1% HCOOH) to give the desired product (18.2 mg, yield: 23%). LC/MS (ESI): m/z 379 [M+H]⁺. ¹H NMR (400 MHz, DMSO) δ 12.25 (br, 1H), 8.26 (s, 1H), 7.63 (d, J=1.9 Hz, 1H), 6.88 (s, 1H), 6.80 (d, J=1.9 Hz, 1H), 6.51 (s, 1H), 4.63-4.52 (m, 1H), 4.31-4.20 (m, 1H), 4.00 (dd, J=11.3, 3.3 Hz, 1H), 3.85 (s, 3H), 3.80-3.76 (m, 1H), 3.69-3.64 (m, 1H), 3.54-3.49 (m, 1H), 3.26-3.23 (m, 1H), 2.24 (s, 3H), 1.28 (d, J=6.7 Hz, 3H).

### Example 19

### Synthesis of (R)-4-(3-(3-cyclopropyl-1H-pyrazol-5-yl)-7-(1-(methylsulfonyl)cyclopropyl)pyrazolo[1,5-a]pyrimidin-5-yl)-3-methylmorpholine

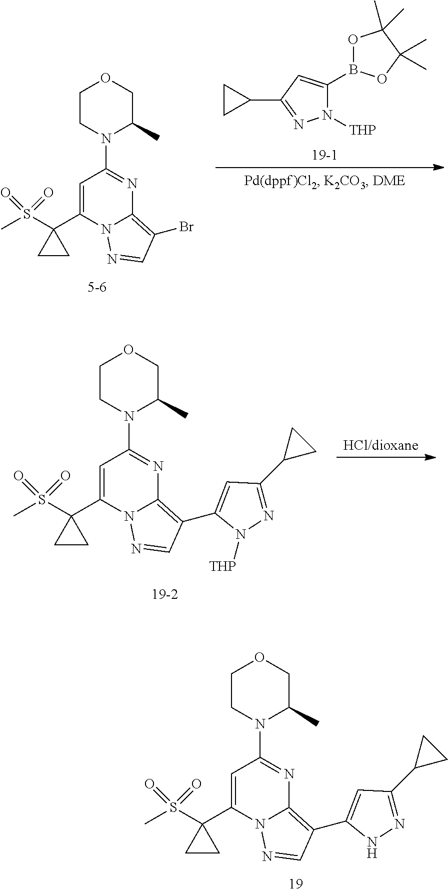

### Step 1. (3R)-4-(3-(3-cyclopropyl-1-(tetrahydro-2H-pyran-2-yl)-1H-pyrazol-5-yl)-7-(1-(methylsulfonyl)cyclopropyl)pyrazolo[1,5-a]pyrimidin-5-yl)-3-methylmorpholine

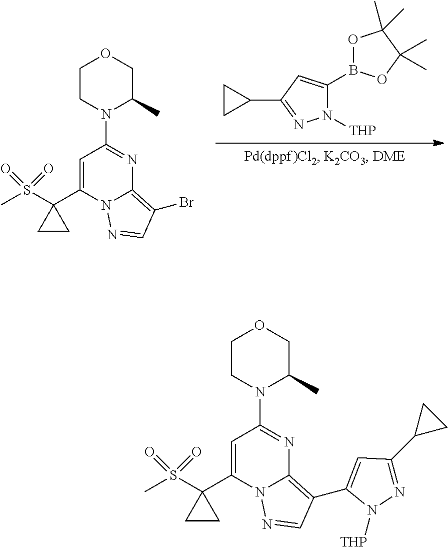

To a solution of (3R)-4-[3-bromo-7-(1-methanesulfonylcyclopropyl)pyrazolo[1,5-a] pyrimidin-5-yl]-3-methylmorpholine (100 mg, 0.24 mmol) and [3-cyclopropyl-1-(oxan-2-yl)-1H-pyrazol-5-yl]boronic acid (142.1 mg, 0.60 mmol) in DME (5 mL) was added K₂CO₃ (2M in H₂O, 0.36 mL, 0.72 mmol) and Pd(dppf)Cl₂ (17.62 mg, 0.024 mmol). The mixture was stirred at 100° C. for 4 h under nitrogen atmosphere. LC-MS showed the reaction was complete. The reaction mixture was diluted with EA (40 mL), then washed with water and brine, dried over anhydrous Na₂SO₄, filtered and concentrated. The residue was purified by column chromatography on silica gel (PE:EA=3:1, V/V) to afford the desired product (60 mg, yield: 47%). LC/MS (ESI): m/z 527 [M+H]⁺.

### Step 2. (R)-4-(3-(3-cyclopropyl-1H-pyrazol-5-yl)-7-(1-(methylsulfonyl)cyclopropyl) pyrazolo[1,5-a]pyrimidin-5-yl)-3-methylmorpholine

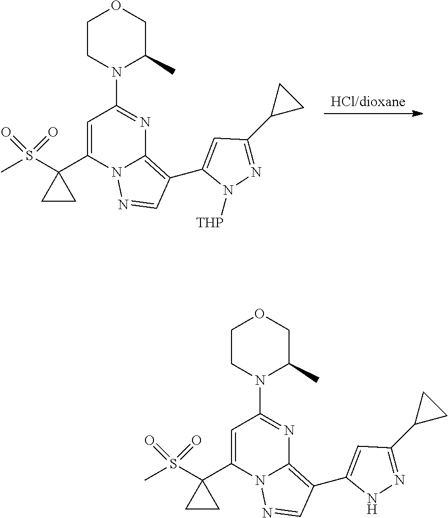

To a solution of (3R)-4-(3-(3-cyclopropyl-1-(tetrahydro-2H-pyran-2-yl)-1H-pyrazol-5-yl)-7-(1-(methylsulfonyl)cyclopropyl)pyrazolo[1,5-a]pyrimidin-5-yl)-3-methylmorpholine (60 mg, 0.11 mmol) in DCM (2 mL) was added HCl solution (4M in dioxane, 2 mL). The mixture was stirred at room temperature for 1 h. LC-MS showed the reaction was complete. The reaction mixture was concentrated under vacuo. The residue was purified by Pre-HPLC (C18, 20-95%, acetonitrile in H₂O with 0.1% HCOOH) to afford the desired product (30 mg, yield: 59%). LC/MS (ESI) m/z: 443 [M+H]⁺. ¹H NMR (400 MHz, DMSO) δ 12.29 (s, 1H), 8.29 (s, 1H), 6.97 (s, 1H), 6.39 (d, J=39.4 Hz, 1H), 4.57 (s, 1H), 4.24 (s, 1H), 4.05-3.95 (m, 1H), 3.79 (d, J=11.5 Hz, 1H), 3.66 (dd, J=11.5, 2.9 Hz, 1H), 3.55-3.46 (m, 1H), 3.29-3.20 (m, 1H), 3.15 (s, 3H), 1.90 (s, 1H), 1.88 (dd, J=7.6, 5.4 Hz, 2H), 1.64 (q, J=5.7 Hz, 2H), 1.26 (d, J=6.7 Hz, 3H), 0.87 (s, 2H), 0.69 (s, 2H).

### Example 20

### Synthesis of (R)—N-methyl-N-(5-(3-methylmorpholino)-3-(1H-pyrazol-5-yl)pyrazolo[1,5-a]pyrimidin-7-yl)methanesulfonamide

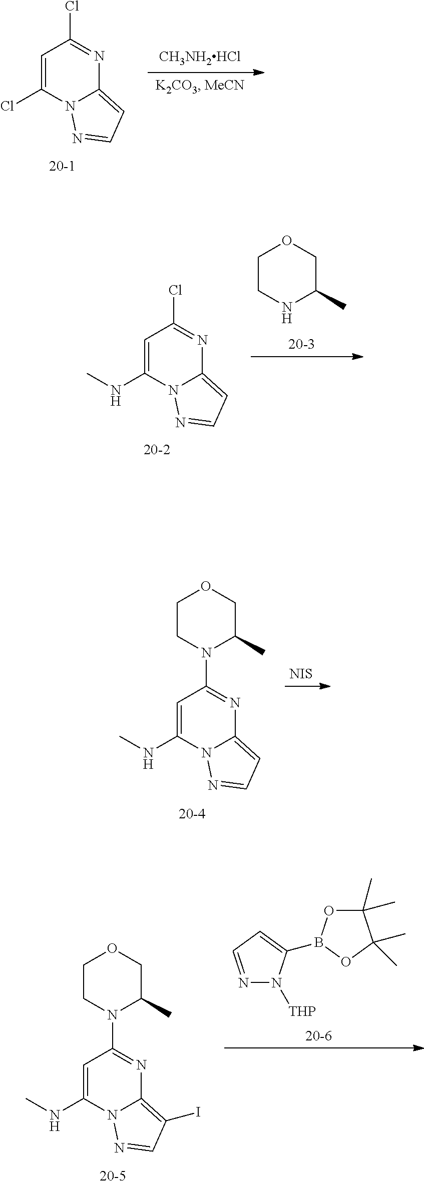

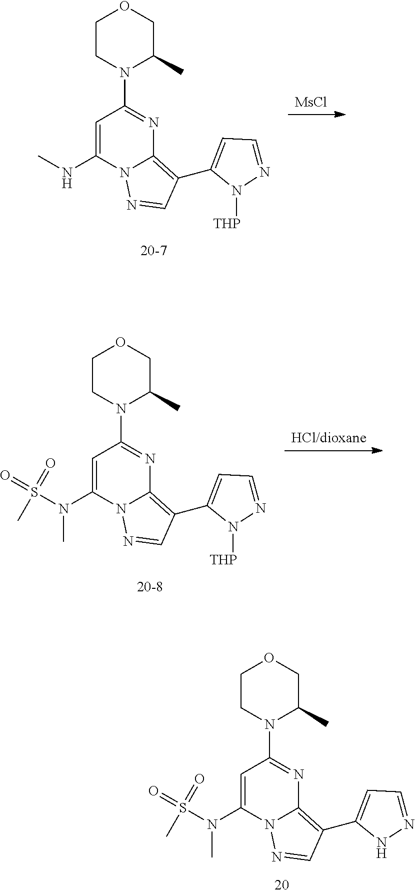

### Step 1. 5-chloro-N-methylpyrazolo[1,5-a]pyrimidin-7-amine

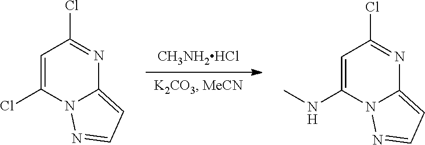

To a solution of 5,7-dichloropyrazolo[1,5-a]pyrimidine (400 mg, 2.13 mmol) in MeCN (4 mL) were added CH₃NH₂·HCl (215.5 mg, 3.19 mmol) and K₂CO₃ (882.1 mg, 6.38 mmol). The mixture was stirred at 80° C. overnight. LC-MS showed the reaction was complete. The reaction mixture was diluted with EA (40 mL), then washed with water and brine, dried over anhydrous Na₂SO₄, filtered and concentrated. The residue was purified by flash chromatography on silica gel (PE:EA=3:1, V/V) to afford the desired product (380 mg, yield: 98%). LC/MS (ESI) m/z: 183 [M+H]⁺.

### Step 2. (R)—N-methyl-5-(3-methylmorpholino)pyrazolo[1,5-a]pyrimidin-7-amine

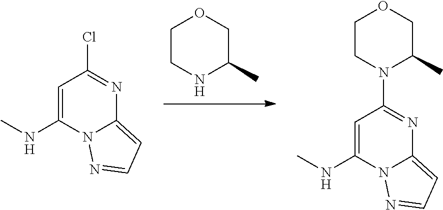

To a solution of 5-chloro-N-methylpyrazolo[1,5-a]pyrimidin-7-amine (150 mg, 0.82 mmol) in NMP (3 mL) were added (3R)-3-methylmorpholine (249.3 mg, 2.46 mmol) and K₂CO₃ (227.1 mg, 1.64 mmol). The mixture was stirred at 200° C. for 1 h under microwave irradiation. LC-MS showed the reaction was complete. The reaction mixture was diluted with EA (40 mL), then washed with water and brine, dried over anhydrous Na₂SO₄, filtered and concentrated. The residue was purified by flash chromatography on silica gel (PE:EA=3:1, V/V) to afford the desired product (125 mg, yield: 55%). LCMS m/z 248 [M+H]⁺.

### Step 3. (R)-3-iodo-N-methyl-5-(3-methylmorpholino)pyrazolo[1,5-a]pyrimidin-7-amine

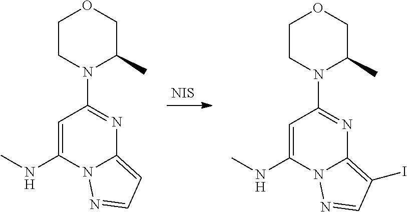

To a solution of N-methyl-5-[(3R)-3-methylmorpholin-4-yl]pyrazolo[1,5-a]pyrimidin-7-amine (175 mg, 0.71 mmol) in MeCN (6 mL) were added 1-Iodpyrrolidin-2,5-dionamine (122.4 mg, 0.71 mmol). The mixture was stirred at room temperature for 1 h. LC-MS showed the reaction was complete. The reaction mixture was diluted with EA (40 mL), then washed with saturated Na₂S₂O₃ aqueous solution and brine, dried over anhydrous Na₂SO₄, filtered and concentrated. The residue was purified by flash chromatography on silica gel (PE:EA=3:1, V/V) to afford the desired product (200 mg, yield: 75%). LC/MS (ESI) m/z: 374 [M+H]⁺.

### Step 4. N-methyl-5-((R)-3-methylmorpholino)-3-(1-(tetrahydro-2H-pyran-2-yl)-1H-pyrazol-5-yl)pyrazolo[1,5-a]pyrimidin-7-amine

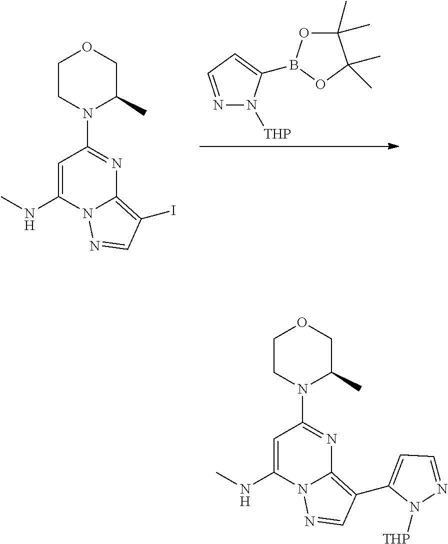

To a solution of (R)-3-iodo-N-methyl-5-(3-methyl morpholino)pyrazolo[1,5-a]pyrimidin-7-amine (180 mg, 0.48 mmol) in co-solvent of dioxane (5 mL) and H₂O (1 mL) were added 1-(oxan-2-yl)-5-(tetramethyl-1,3,2-dioxaborolan-2-yl)-1H-pyrazole (335.4 mg, 1.21 mmol), K₂CO₃ (133.3 mg, 0.97 mmol) and Pd(dppf)Cl₂ (70.6 mg, 0.10 mmol). The mixture was stirred at 100° C. overnight under nitrogen atmosphere. LC-MS showed the reaction was complete. The reaction mixture was diluted with EA (40 mL), then washed with water and brine, dried over anhydrous Na₂SO₄, filtered and concentrated. The residue was purified by flash chromatography on silica gel (PE:EA=2:1, V/V) to afford the desired product (70 mg, yield: 36%). LC/MS (ESI) m/z: 398 [M+H]⁺.

### Step 5. N-methyl-N-(5-((R)-3-methylmorpholino)-3-(1-(tetrahydro-2H-pyran-2-yl)-1H-pyrazol-5-yl)pyrazolo[1,5-a]pyrimidin-7-yl)methanesulfonamide

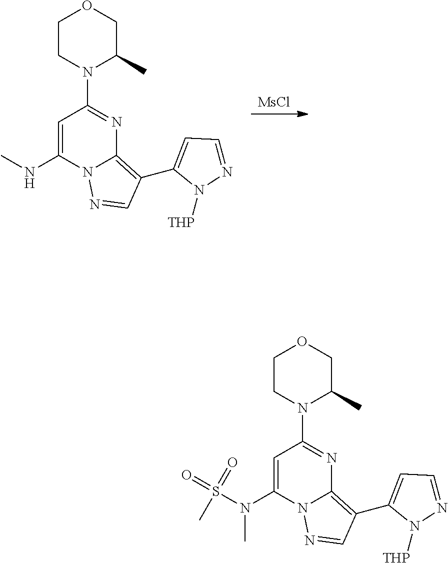

To solution of N-methyl-5-((R)-3-methylmorpholino)-3-(1-(tetrahydro-2H-pyran-2-yl)-1H-pyrazol-5-yl)pyrazolo[1,5-a]pyrimidin-7-amine (53 mg, 0.13 mmol) in THF (2 mL) at −78° C. was added LDA (2 M in THF, 0.2 mL, 0.40 mmol) drop wise. The mixture was stirred at −78° C. for 30 min, then a solution of methanesulfonyl chloride (0.03 mL, 0.33 mmol) in THF (0.5 mL) was added drop wise. The resulting mixture was stirred at room temperature for 1 h. LC-MS showed the reaction was complete. The reaction mixture was quenched with saturated NH₄Cl aqueous solution and extracted with EA (30 mL×2). The combined organic layer was washed with brine, dried over anhydrous Na₂SO₄, filtered and concentrated. The residue was purified by flash chromatography on silica gel (DCM:MeOH=30:1, V/V) to afford the desired product (38 mg, yield: 59%). LC/MS (ESI) (m/z): 476 [M+H]⁺.

### Step 6. (R)—N-methyl-N-(5-(3-methylmorpholino)-3-(1H-pyrazol-5-yl)pyrazolo[1,5-a]pyrimidin-7-yl)methanesulfonamide

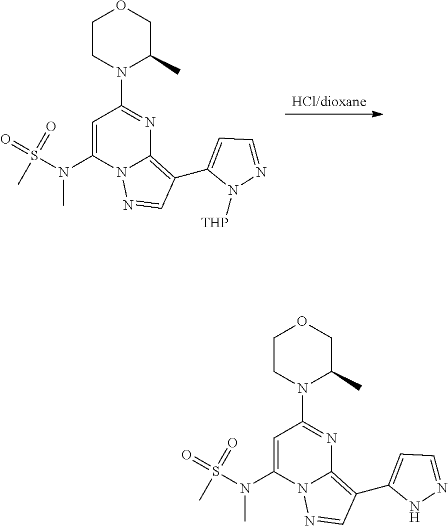

A mixture of N-methyl-N-(5-((R)-3-methylmorpholino)-3-(1-(tetrahydro-2H-pyran-2-yl)-1H-pyrazol-5-yl)pyrazolo[1,5-a]pyrimidin-7-yl)methanesulfonamide (50 mg, 0.11 mmol) in HCl solution (4 M in dioxane, 2 mL) was stirred at room temperature for 2 h. LC-MS showed the reaction was complete. The reaction mixture was concentrated under vacuo. The residue was purified by Pre-HPLC (Cis, 10-95%, MeOH in H₂O with 0.1% HCOOH) to afford the desired product (10.6 mg, yield: 25%). LC/MS (ESI) m/z: 392 [M+H]⁺. ¹H NMR (400 MHz, DMSO) δ 12.75 (d, J=81.4 Hz, 1H), 8.31 (s, 1H), 7.56 (s, 1H), 6.84 (s, 1H), 6.73 (s, 1H), 4.58 (s, 1H), 4.21 (d, J=12.2 Hz, 1H), 4.00 (dd, J=11.4, 3.3 Hz, 1H), 3.78 (d, J=11.4 Hz, 1H), 3.66 (dd, J=11.5, 2.9 Hz, 1H), 3.51 (td, J=11.9, 2.9 Hz, 1H), 3.44 (d, J=4.2 Hz, 3H), 3.40 (s, 3H), 3.31-3.21 (m, 1H), 1.27 (d, J=6.7 Hz, 3H).

### Example 21

### Synthesis of (R)-3-methyl-4-(8-(1-methyl-1H-pyrazol-5-yl)-3-(1H-pyrazol-5-yl)imidazo[1,2-b]pyridazin-6-yl)morpholine

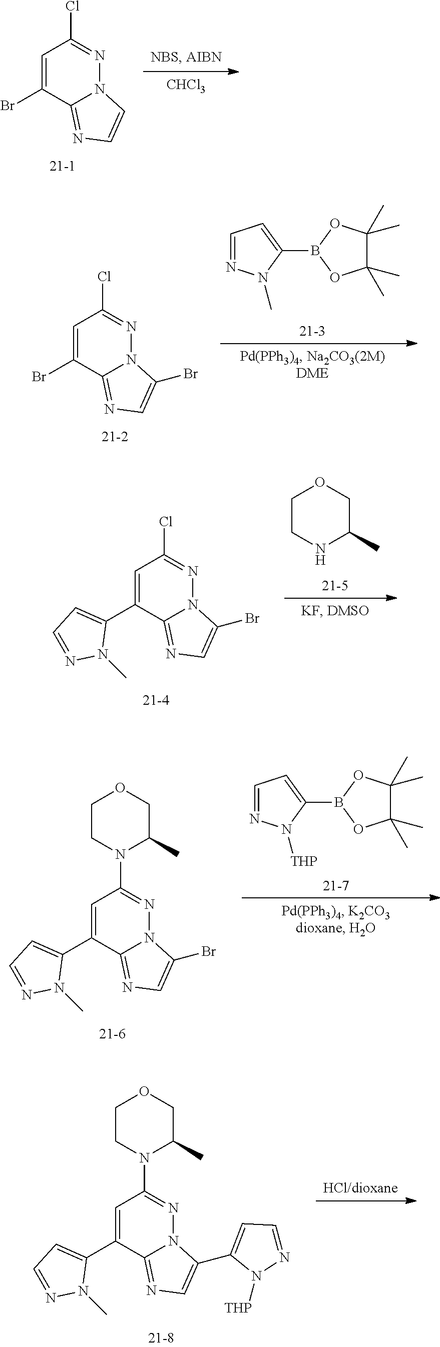

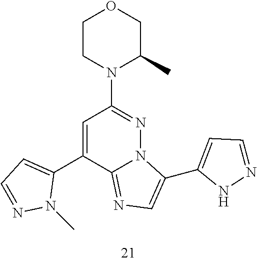 [SMILES: C[C@@H]1COCCN1c1cc(-c2ccnn2C)c2ncc(-c3ccn[nH]3)n2n1]

### Step 1. 3,8-dibromo-6-chloroimidazo[1,2-b]pyridazine

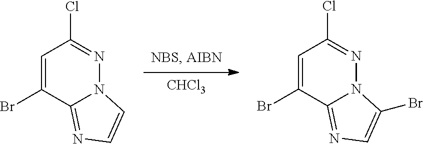

To a solution of 8-bromo-6-chloroimidazo[1,2-b]pyridazine (1.1 g, 4.73 mmol) and AIBN (80 mg, 0.47 mmol) in CHCl₃ (50 mL) was added NBS (1.68 g, 9.46 mmol) portion wise. The mixture was stirred at 80° C. for 3 h. LC-MS showed the reaction was complete. The reaction mixture was concentrated under vacuo to dryness. The residue was purified by flash chromatography on silica gel (PE:EA=10:1, V/V) to give the desired product (580 mg, yield: 39%/). LC/MS (ESI): m/z 310/312/314 [M+H]⁺.

### Step 2. 3-bromo-6-chloro-8-(1-methyl-1H-pyrazol-5-yl)imidazo[1,2-b]pyridazine

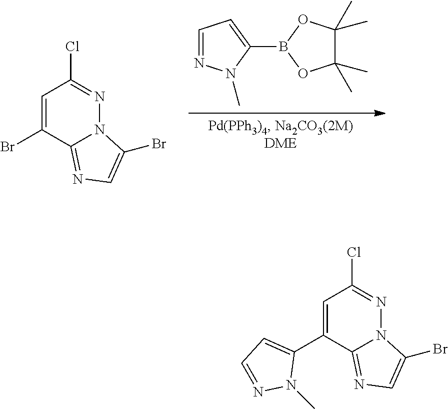 [SMILES: Clc1cc(Br)c2ncc(Br)n2n1 ; Cn1nccc1B1OC(C)(C)C(C)(C)O1 ; "Pd(PPh3)4, Na2CO3(2M)DME" ; Cn1nccc1-c1cc(Cl)nn2c(Br)cnc12]

To a solution of 3,8-dibromo-6-chloroimidazo[1,2-b]pyridazine (350 mg, 1.12 mmol), 1-methyl-5-(tetramethyl-1,3,2-dioxaborolan-2-yl)-1H-pyrazole (468 mg, 2.25 mmol) and Na₂CO₃ (2M in H₂O, 1.7 mL, 3.38 mmol) in DME (10 mL) was added Pd(PPh₃)₄ (130 mg, 0.11 mmol). The mixture was stirred at 90° C. for 16 h under N₂ atmosphere. LC-MS showed the reaction was complete. The reaction mixture was diluted with EA (50 mL), then washed with water and brine, dried over anhydrous Na₂SO₄, filtered and concentrated. The residue was purified by flash chromatography on silica gel (PE:EA=5:1, V/V) to give the desired product (250 mg, yield: 71%). LC/MS (ESI): m/z 312/314 [M+H]⁺.

### Step 3. (R)-4-(3-bromo-8-(1-methyl-1H-pyrazol-5-yl)imidazo[1,2-b]pyridazin-6-yl)-3-methylmorpholine

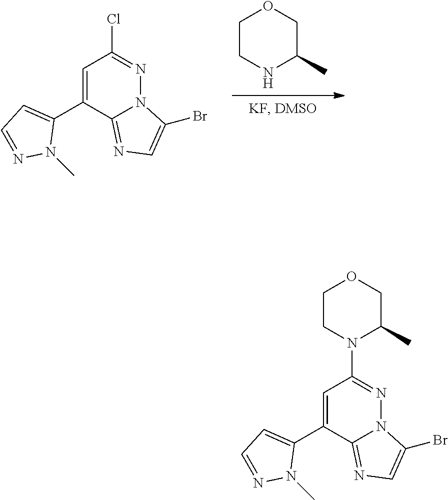

To a solution of 3-bromo-6-chloro-8-(1-methyl-1H-pyrazol-5-yl)imidazo[1,2-b]pyridazine (170 mg, 0.54 mmol) and KF (158 mg, 2.72 mmol) in DMSO (17 mL) was added (3R)-3-methylmorpholine (550 mg, 5.44 mmol). The mixture was stirred at 180° C. for 1 h under microwave irradiation. LC-MS showed the reaction was complete. The reaction mixture was diluted with EA (60 mL), then washed with water (20 mL×3) and brine, dried over anhydrous Na₂SO₄, filtered and concentrated. The residue was purified by flash chromatography on silica gel (PE:EA=3:1, V/V) to give the desired product (65 mg, yield: 32%). LC/MS (ESI): m/z 377/379 [M+H]⁺.

### Step 4. (3R)-3-methyl-4-(8-(1-methyl-1H-pyrazol-5-yl)-3-(1-(tetrahydro-2H-pyran-2-yl)-1H-pyrazol-5-yl)imidazo[1,2-b]pyridazin-6-yl)morpholine

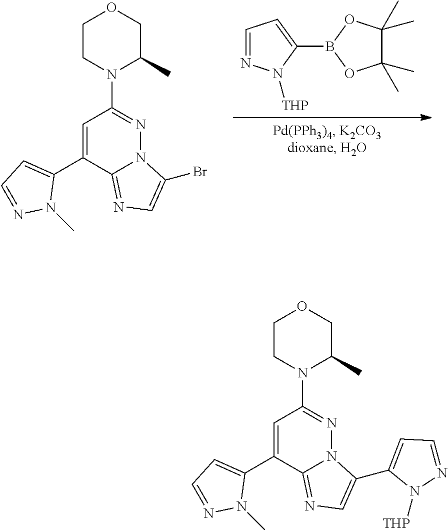

To a solution of (R)-4-(3-bromo-8-(1-methyl-1H-pyrazol-5-yl)imidazo[1,2-b]pyridazin-6-yl)-3-methylmorpholine (65 mg, 0.17 mmol), 1-(oxan-2-yl)-5-(tetra methyl-1,3,2-dioxaborolan-2-yl)-1H-pyrazole (96 mg, 0.35 mmol), K₂CO₃ (71 mg, 0.52 mmol) in co-solvent of dioxane (3 mL) and H₂O (0.6 mL) was added Pd(PPh₃)₄ (20 mg, 0.02 mmol). The mixture was stirred at 100° C. for 16 h under N₂ atmosphere. LC-MS showed the reaction was complete. The reaction mixture was diluted with EA (60 mL), then washed with water and brine, dried over anhydrous Na₂SO₄, filtered and concentrated. The residue was purified by flash chromatography on silica gel (PE:EA=2:1, V/V) to give the desired product (55 mg, yield: 71%). LC/MS (ESI): m/z 449 [M+H]⁺.

### Step 5. (R)-3-methyl-4-(8-(1-methyl-1H-pyrazol-5-yl)-3-(1H-pyrazol-5-yl)imidazo [1,2-b]pyridazin-6-yl)morpholine

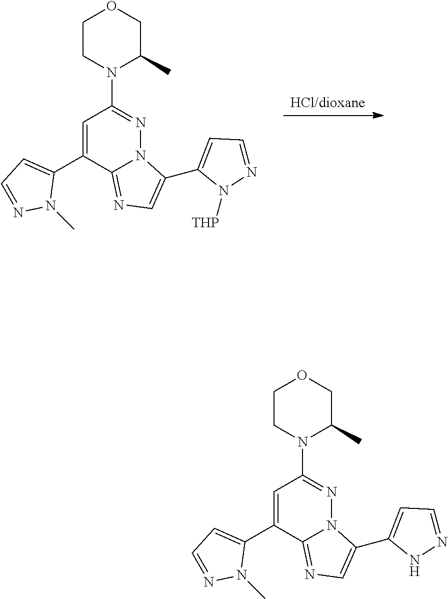

A mixture of (3R)-3-methyl-4-(8-(1-methyl-1H-pyrazol-5-yl)-3-(1-(tetrahydro-2H-pyran-2-yl)-1H-pyrazol-5-yl)imidazo[1,2-b]pyridazin-6-yl)morpholine (55 mg, 0.12 mmol) in HCl solution (4M in dioxane, 2 mL) was stirred at room temperature for 1 h. LC-MS showed the reaction was complete. The reaction mixture was concentrated under vacuo to dryness. The residue was purified by Prep-HPLC (C₁₈, 10-95%, MeOH in H₂O with 0.1% HCOOH) to give the desired product (6 mg, yield: 13%). LC/MS (ESI): m/z 365 [M+H]⁺. ¹H NMR (400 MHz, DMSO) δ 13.31 (d, J=114.0 Hz, 1H), 8.15-7.64 (m, 3H), 7.32 (d, J=22.6 Hz, 1H), 7.14 (d, J=26.1 Hz, 1H), 6.88 (d, J=1.9 Hz, 1H), 4.43 (dd, J=10.0, 4.4 Hz, 1H), 4.08 (dd, J=11.4, 3.0 Hz, 1H), 4.03 (s, 3H), 3.94 (d, J=12.6 Hz, 1H), 3.82 (dt, J=11.6, 7.0 Hz, 2H), 3.69-3.61 (m, 1H), 3.34 (dd, J=12.3, 3.7 Hz, 1H), 1.32 (d, J=6.7 Hz, 3H).

### Example 22

### Synthesis of (R)-3-methyl-4-(8-(1-(methylsulfonyl)cyclopropyl)-3-(1H-pyrazol-5-yl)imidazo[1,2-b]pyridazin-6-yl)morpholine

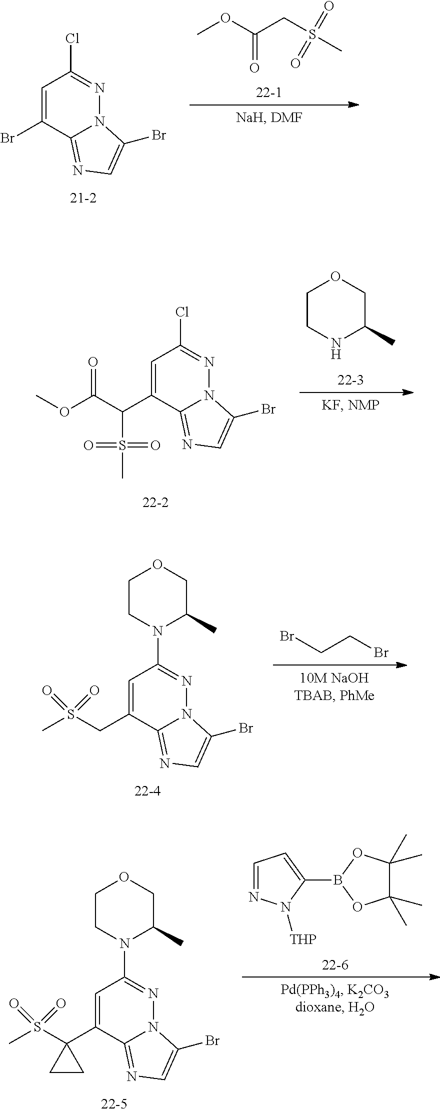

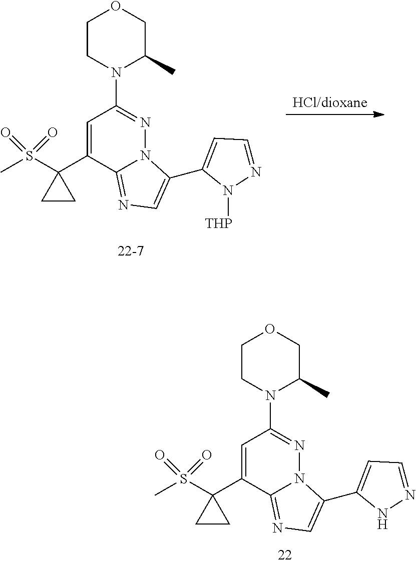

### Step 1. Methyl 2-(3-bromo-6-chloroimidazo[1,2-b]pyridazin-8-yl)-2-(methylsulfonyl) acetate

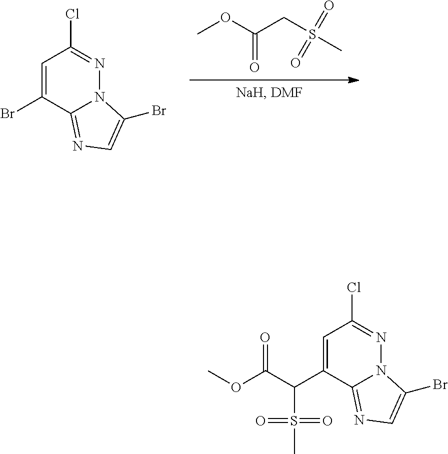

To a solution of methyl 2-methanesulfonylacetate (340 mg, 2.24 mmol) in DMF (10 mL) at 0° C. was added NaH (60%, 149 mg, 3.73 mmol) portion wise. The mixture was stirred at 0° C. for 20 min, then a solution of 3,8-dibromo-6-chloroimidazo[1,2-b]pyridazine (580 mg, 1.86 mmol) in DMF (1 mL) was added. The resulting mixture was stirred at 0° C. for 2 h. LC-MS showed the reaction was complete. The reaction mixture was quenched with saturated NH₄Cl aqueous solution, then extracted with EA (30 mL×2). The combined organic layer was washed with brine, dried over anhydrous Na₂SO₄, filtered and concentrated. The residue was purified by flash chromatography on silica gel (PE:EA=10:1, V/V) to afford the desired product (680 mg, yield: 95%). LC/MS (ESI): m/z 382/384 [M+H]⁺.

### Step 2. (R)-4-(3-bromo-8-((methylsulfonyl)methyl)imidazo[1,2-b]pyridazin-6-yl)-3-methylmorpholine

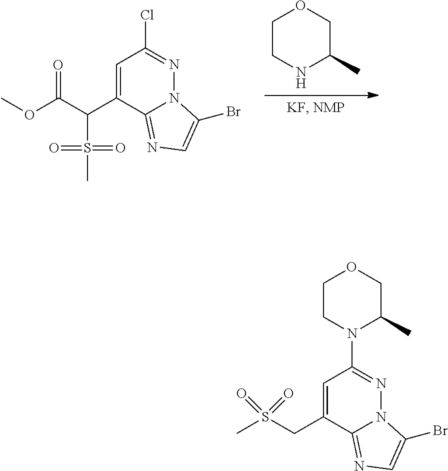

To a solution of methyl 2-{3-bromo-6-chloroimidazo[1,2-b]pyridazin-8-yl}-2-methanesulfonylacetate (300 mg, 0.784 mmol) and (3R)-3-methylmorpholine (397 mg, 3.92 mmol) in NMP (5 mL) was added KF (91 mg, 1.57 mmol). The mixture was stirred at 180° C. for 1 h under microwave irradiation. LC-MS showed the reaction was complete. The mixture was diluted with EA (40 mL), then washed with water and brine, dried over anhydrous Na₂SO₄, filtered and concentrated. The residue was purified by flash chromatography on silica gel (PE:EA=5:1, V/V) to afford the desired product (110 mg, yield: 36%). LC/MS (ESI): m/z 389/391[M+H]⁺.

### Step 3. (R)-4-(3-bromo-8-(1-(methylsulfonyl)cyclopropyl)imidazo[1,2-b]pyridazin-6-yl)-3-methylmorpholine

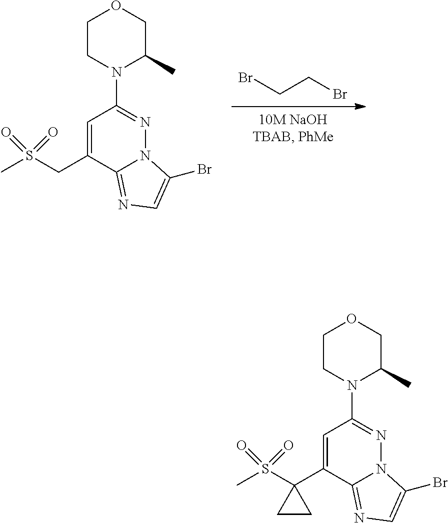

To a solution of (R)-4-(3-bromo-8-((methylsulfonyl)methyl)imidazo[1,2-b]pyridazin-6-yl)-3-methylmorpholine (110 mg, 0.28 mmol), 1,2-dibromoethane (0.06 mL, 0.71 mmol) and TBAB (18 mg, 0.06 mmol) in Toluene (8 mL) was added NaOH (10 M in H₂O, 0.28 mL, 2.83 mmol). The mixture was stirred at 60° C. for 2 h. LC-MS showed the reaction was complete. The mixture was diluted with EA (40 mL), then washed with water and brine, dried over anhydrous Na₂SO₄, filtered and concentrated. The residue was purified by flash chromatography on silica gel (PE:EA=3:1, V/V) to afford the desired product (50 mg, yield: 43%). LC/MS (ESI): m/z 415/417 [M+H]⁺.

### Step 4. (3R)-3-methyl-4-(8-(1-(methylsulfonyl)cyclopropyl)-3-(1-(tetrahydro-2H-pyran-2-yl)-1H-pyrazol-5-yl)imidazo[1,2-b]pyridazin-6-yl)morpholine

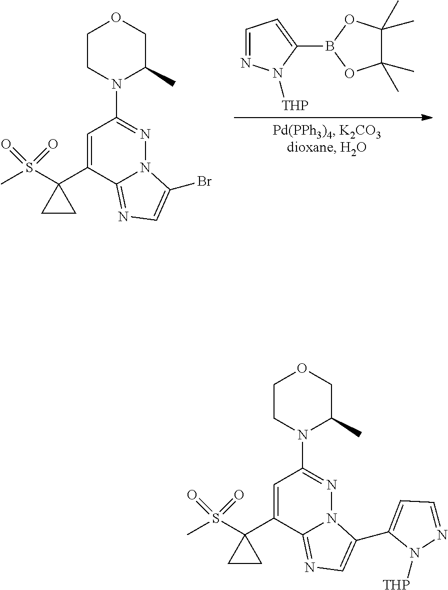

To a solution of (R)-4-(3-bromo-8-(1-(methylsulfonyl)cyclopropyl)imidazo[1,2-b]pyridazin-6-yl)-3-methylmorpholine (50 mg, 0.12 mmol), 1-(oxan-2-yl)-5-(tetra methyl-1,3,2-dioxaborolan-2-yl)-1H-pyrazole (67 mg, 0.24 mmol), K₂CO₃ (50 mg, 0.36 mmol) in co-solvent of dioxane (3 mL) and H₂O (0.6 mL) was added Pd(PPh₃)₄ (14 mg, 0.012 mmol). The mixture was stirred at 100° C. for 16 h under N₂ atmosphere. LC-MS showed the reaction was complete. The mixture was diluted with EA (40 mL), then washed with water and brine, dried over anhydrous Na₂SO₄, filtered and concentrated. The residue was purified by flash chromatography on silica gel (PE:EA=2:1, V/V) to afford the desired product (55 mg, yield: 94%). LC/MS (ESI): m/z 487 [M+H]⁺.

### Step 5. (R)-3-methyl-4-(8-(1-(methylsulfonyl)cyclopropyl)-3-(1H-pyrazol-5-yl)imidazo[1,2-b]pyridazin-6-yl)morpholine

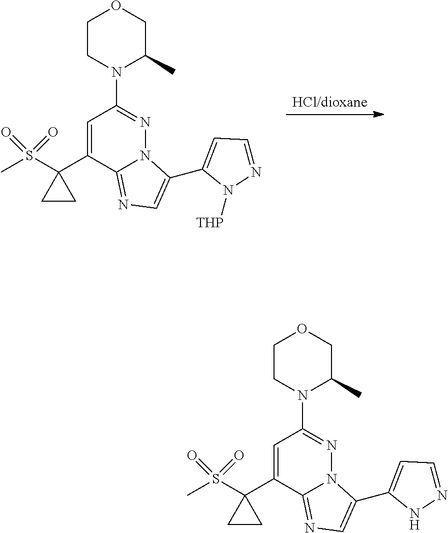

A mixture of (3R)-3-methyl-4-(8-(1-(methylsulfonyl)cyclopropyl)-3-(1-(tetrahydro-2H-pyran-2-yl)-1H-pyrazol-5-yl)imidazo[1,2-b]pyridazin-6-yl)morpholine (55 mg, 0.11 mmol) in HCl solution (4M in dioxane, 3 mL) was stirred at room temperature for 1 h. LC-MS showed the reaction was complete. The reaction mixture was concentrated under vacuo. The residue was purified by Prep-HPLC (C₁₈, 10-95%, MeOH in H₂O with 0.1% HCOOH) to give the desired product (4.8 mg, yield: 11%). LC/MS (ESI): m/z 403 [M+H]⁺. ¹H NMR (400 MHz, DMSO) δ 13.09 (s, 1H), 7.99 (t, J=71.7 Hz, 2H), 7.33 (s, 1H), 7.05 (s, 1H), 4.39-4.27 (m, 1H), 4.01 (dd, J=11.4, 3.0 Hz, 1H), 3.77 (ddd, J=19.6, 13.7, 7.6 Hz, 3H), 3.58 (td, J=11.7, 2.7 Hz, 1H), 3.26-3.22 (m, 1H), 3.14 (s, 3H), 1.81 (q, J=5.0 Hz, 2H), 1.57 (q, J=5.4 Hz, 2H), 1.23 (d, J=6.7 Hz, 3H).

### Example 23

### Synthesis of (R)-3-methyl-4-(8-(1-methyl-1H-pyrazol-5-yl)-3-(3-methyl-1H-pyrazol-5-yl)imidazo[1,2-b]pyridazin-6-yl)morpholine

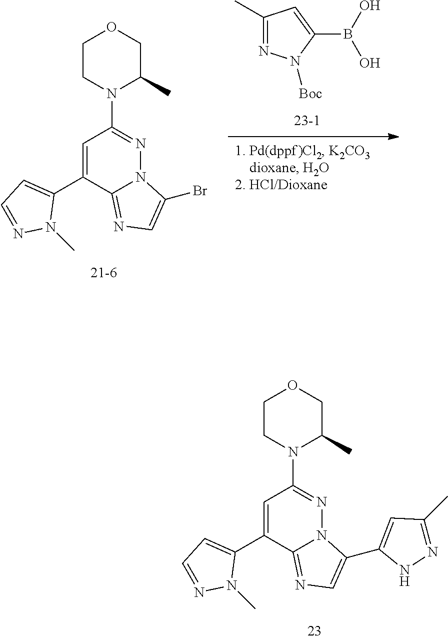 [SMILES: C[C@@H]1COCCN1c1cc(-c2ccnn2C)c2ncc(Br)n2n1 ; Cc1cc(B(O)O)n(C(=O)OC(C)(C)C)n1 ; "1. Pd(dppf)Cl2, K2CO3    dioxane, H2O2. HCl/Dioxane" ; Cc1cc(-c2cnc3c(-c4ccnn4C)cc(N4CCOC[C@H]4C)nn23)[nH]n1]

### Step 1. (R)-3-methyl-4-(8-(1-methyl-1H-pyrazol-5-yl)-3-(3-methyl-1H-pyrazol-5-yl)imidazo[1,2-b]pyridazin-6-yl)morpholine

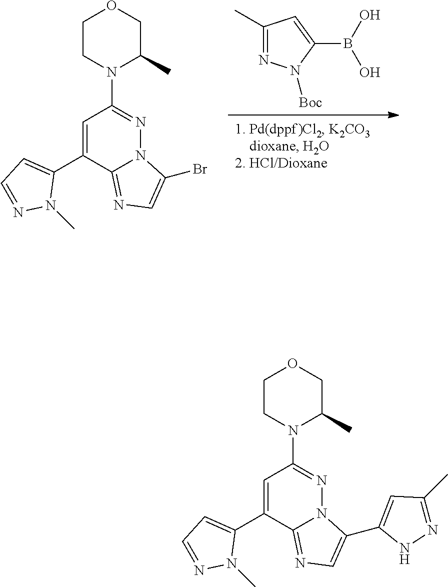 [SMILES: C[C@@H]1COCCN1c1cc(-c2ccnn2C)c2ncc(Br)n2n1 ; Cc1cc(B(O)O)n(C(=O)OC(C)(C)C)n1 ; "1. Pd(dppf)Cl2, K2CO3    dioxane, H2O2. HCl/Dioxane" ; Cc1cc(-c2cnc3c(-c4ccnn4C)cc(N4CCOC[C@H]4C)nn23)[nH]n1]

To a mixture of (R)-4-(3-bromo-8-(1-methyl-1H-pyrazol-5-yl)imidazo[1,2-b]pyridazin-6-yl)-3-methylmorpholine (50 mg, 0.13 mmol), {1-[(tert-butoxy) carbonyl]-3-methyl-1H-pyrazol-5-yl}boronic acid (60 mg, 0.27 mmol), K₂CO₃ (55 mg, 0.40 mmol) in co-solvent of dioxane (2.5 mL) and H₂O (0.5 mL) was added Pd(dppf)Cl₂ (5 mg, 0.01 mmol). The mixture was stirred at 100° C. for 16 h under N₂ atmosphere. LC-MS showed the reaction was complete. The reaction mixture was diluted with EA (40 mL), then washed with water and brine, dried over anhydrous Na₂SO₄, filtered and concentrated. The residue was dissolved in DCM (2 mL), then HCl solution (4M in dioxane, 1 mL) was added. The mixture was stirred at room temperature for 1 h. LC-MS showed the reaction was complete. The reaction mixture was concentrated under vacuo. The residue was purified by Prep-HPLC (C₁₈, 10-95%, MeOH in H₂O with 0.1% HCOOH) to give the desired product (6 mg, yield: 12%).

LC/MS (ESI): m/z 379 [M+H]⁺. ¹H NMR (400 MHz, DMSO) δ 12.79 (s, 1H), 7.93 (s, 1H), 7.60 (d, J=1.9 Hz, 1H), 7.23 (s, 1H), 6.83 (s, 1H), 6.81 (d, J=1.9 Hz, 1H), 4.36 (q, J=6.7 Hz, 1H), 4.02 (dd, J=11.3, 3.3 Hz, 1H), 3.97 (s, 3H), 3.91-3.86 (m, 1H), 3.80-3.73 (m, 2H), 3.62-3.57 (m, 1H), 3.30-3.28 (m, 1H), 2.32 (s, 3H), 1.25 (d, J=6.6 Hz, 3H).

### Example 24

### Synthesis of (R)-3-methyl-4-(4-(1-(methylsulfonyl)cyclopropyl)-8-(1H-pyrazol-5-yl)imidazo[1,5-a]pyrimidin-2-yl)morpholine

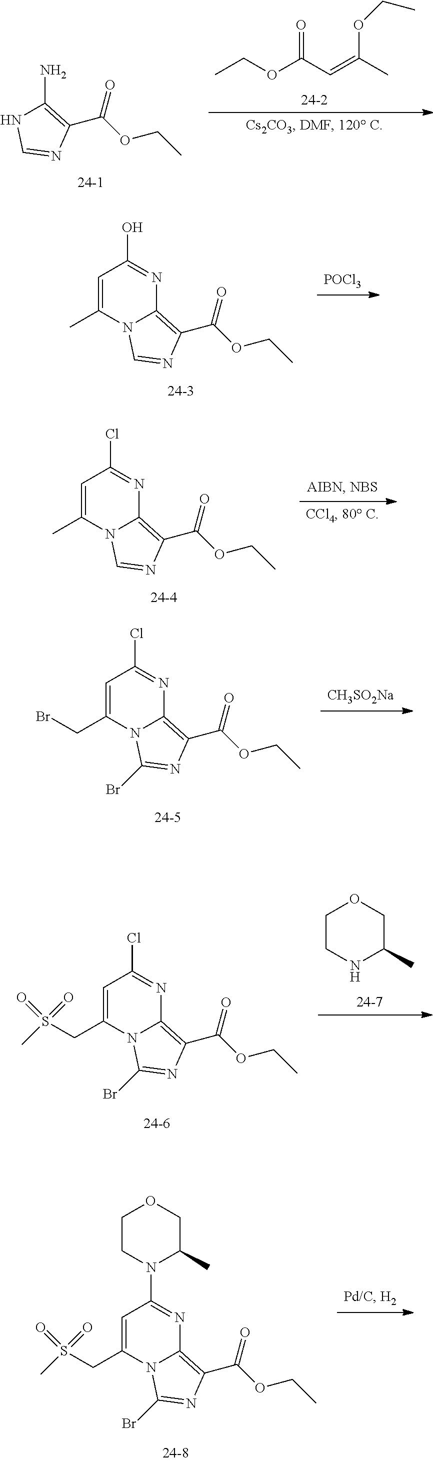

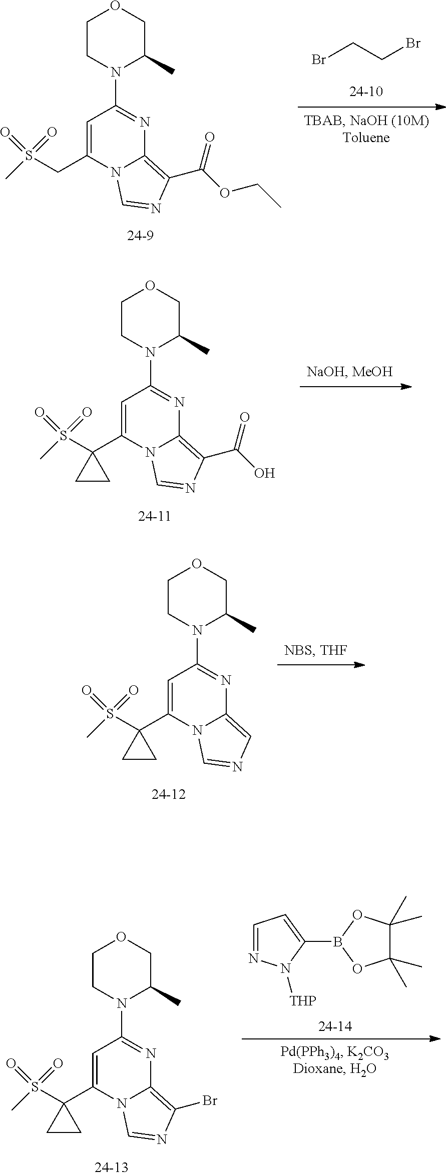

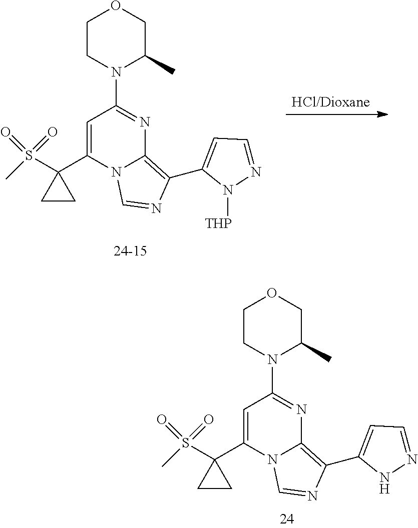

### Step 1. Ethyl 2-hydroxy-4-methylimidazo[1,5-a]pyrimidine-8-carboxylate

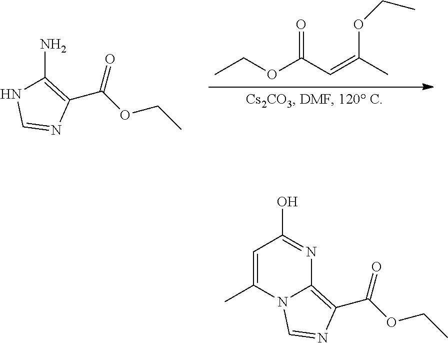

To a suspension of ethyl 5-amino-1H-imidazole-4-carboxylate (2.5 g, 16.11 mmol) and Cs₂CO₃ (10.5 g, 32.22 mmol) in DMF (20 mL) was added ethyl (2Z)-3-ethoxybut-2-enoate (3.06 g, 19.34 mmol). The mixture was stirred at 120° C. for 16 h. LC-MS showed the reaction was complete. The reaction mixture was diluted with DCM (40 mL), then filtered. Then filter cake was washed with DCM and MeOH (4:1, 40 mL). The filtrate was concentrated to give the crude product (3.17 g), which was used in the next step without further purification. LC/MS (ESI): m/z 222 [M+H]⁺.

### Step 2. Ethyl 2-chloro-4-methylimidazo[1,5-a]pyrimidine-8-carboxylate

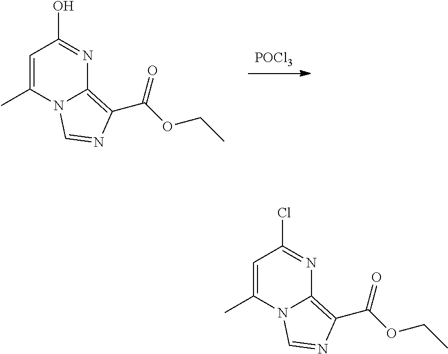 [SMILES: CCOC(=O)c1ncn2c(C)cc(O)nc12 ; "POCl3" ; CCOC(=O)c1ncn2c(C)cc(Cl)nc12]

A mixture of ethyl 2-hydroxy-4-methylimidazo[1,5-a]pyrimidine-8-carboxylate (3.1 g, 14.01 mmol) in POCl₃ (30 mL) was stirred at 100° C. for 2 h.

LC-MS showed the reaction was complete. The reaction mixture was concentrated under reduced pressure. The mixture was diluted with DCM (40 mL), then washed with saturated NaHCO₃ aqueous solution and brine, dried over anhydrous Na₂SO₄, filtered and concentrated. The residue was purified by flash chromatography on silica gel (PE:EA=5:1, V/V) to afford the desired product (2.52 g, yield: 65%). LC/MS (ESI): m/z 240 [M+H]⁺.

### Step 3. Ethyl 6-bromo-4-(bromomethyl)-2-chloroimidazo[1,5-a]pyrimidine-8-carboxylate

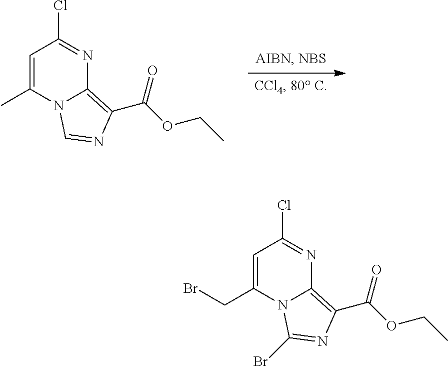

To a solution of ethyl 2-chloro-4-methylimidazo[1,5-a]pyrimidine-8-carboxylate (2.5 g 10.43 mmol) and AIBN (170 mg, 1.04 mmol) in CCl₄ (50 mL) was added NBS (4.3 g, 24.0 mmol). The mixture was stirred at 90° C. for 8 h. LC-MS showed the reaction was complete. The mixture was diluted with DCM (40 mL), then washed with water and brine, dried over anhydrous Na₂SO₄, filtered and concentrated. The residue was purified by flash chromatography on silica gel (PE:EA=5:1, V/V) to afford the desired product (2.75 g, yield: 66%). LC/MS(ESI): m/z 396/398/400 [M+H]⁺.

### Step 4. Ethyl 6-bromo-2-chloro-4-((methylsulfonyl)methyl)imidazo[1,5-a]pyrimidine-8-carboxylate

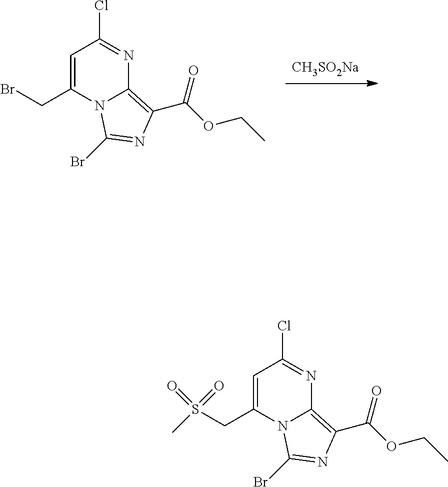 [SMILES: CCOC(=O)c1nc(Br)n2c(CBr)cc(Cl)nc12 ; "CH3SO2Na" ; CCOC(=O)c1nc(Br)n2c(CS(C)(=O)=O)cc(Cl)nc12]

To a solution of ethyl 6-bromo-4-(bromomethyl)-2-chloroimidazo[1,5-a]pyrimidine-8-carboxylate (1 g, 2.52 mmol) in DMF (15 mL) at −60° C. was added methanesulfonylsodium (0.26 g, 2.52 mmol). The mixture was stirred at −60° C. for 1 h. LC-MS showed the reaction was complete. The mixture was diluted with DCM (40 mL), then washed with water and brine, dried over anhydrous Na₂SO₄, filtered and concentrated. The residue was purified by flash chromatography on silica gel (PE:EA=3:1, V/V) to afford the desired product (850 mg, yield: 85%). LC/MS (ESI): m/z 396/398 [M+H]⁺.

### Step 5. Ethyl (R)-6-bromo-2-(3-methylmorpholino)-4-((methylsulfonyl)methyl) imidazo[1,5-a]pyrimidine-8-carboxylate

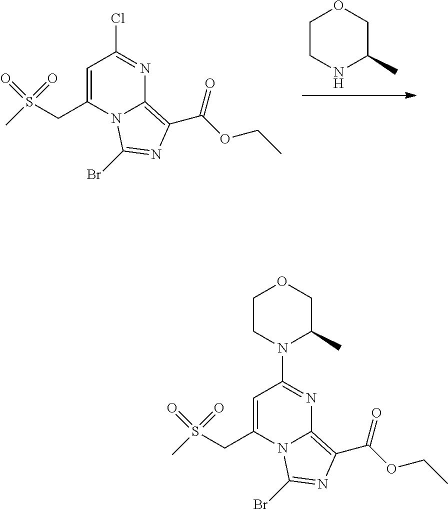

To a solution of ethyl 6-bromo-2-chloro-4-(methanesulfonylmethyl)imidazo[1,5-a]pyrimidine-8-carboxylate (850 mg, 2.14 mmol) in MeCN (15 mL) was added (3R)-3-methylmorpholine (650 mg, 6.43 mmol). The mixture was stirred at 80° C. for 1.5 h. LC-MS showed the reaction was complete. The mixture was concentrated to dryness. The residue was purified by flash chromatography on silica gel (PE:EA=1:1, V/V) to afford the desired product (827 mg, yield: 84%). LC/MS (ESI): m/z 461/463 [M+H]⁺.

### Step 6. Ethyl (R)-2-(3-methylmorpholino)-4-((methylsulfonyl)methyl)imidazo[1,5-a]pyrimidine-8-carboxylate

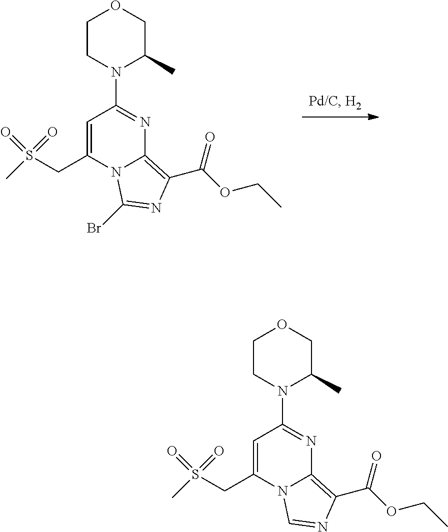

To a solution of ethyl (R)-6-bromo-2-(3-methylmorpholino)-4-((methylsulfonyl)methyl)imidazo[1,5-a]pyrimidine-8-carboxylate (820 mg, 1.78 mmol) in THF (8 mL) was added Pd/C (10%, 200 mg). The mixture was stirred at room temperature for 2 h under H₂ atmosphere. LC-MS showed the reaction was complete. The reaction mixture was filtered, the filtrate was concentrated under vacuo. The residue was purified by flash chromatography on silica gel (PE:EA=1:1, V/V) to afford the desired product (570 mg, yield: 84%). LC/MS (ESI): m/z 383 [M+H]⁺.

### Step 7. (R)-2-(3-methylmorpholino)-4-(1-(methylsulfonyl)cyclopropyl)imidazo[1,5-a]pyrimidine-8-carboxylic acid

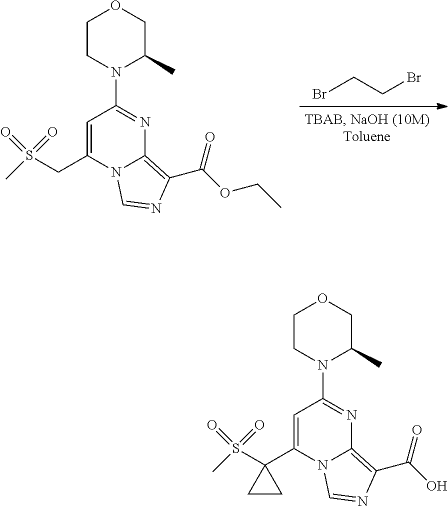

To a solution of ethyl (R)-2-(3-methylmorpholino)-4-((methylsulfonyl)methyl) imidazo[1,5-a]pyrimidine-8-carboxylate (300 mg, 0.78 mmol), 1,2-dibromoethane (0.17 mL, 1.96 mmol) and TBAB (51 mg, 0.16 mmol) in Toluene (10 mL) was added NaOH (10 M in H₂O, 0.78 mL, 7.84 mmol). The mixture was stirred at 60° C. for 16 h. LC-MS showed the reaction was complete. The mixture was concentrated under reduced pressure. The residue was diluted with DCM (50 mL), then adjusted to PH=5 by the addition of HCl solution (1M). The aqueous layer was separated, then extracted with DCM (30 mL×2) twice. The combined organic layer was washed with brine, dried over anhydrous Na₂SO₄, filtered and concentrated. The residue was purified by flash chromatography on silica gel (DCM:MeOH=10:1, V/V) to afford the desired product (298 mg, yield: 99%). LC/MS (ESI): m/z 381 [M+H]⁺.

### Step 8. (R)-3-methyl-4-(4-(1-(methylsulfonyl)cyclopropyl)imidazo[1,5-a]pyrimidin-2-yl)morpholine

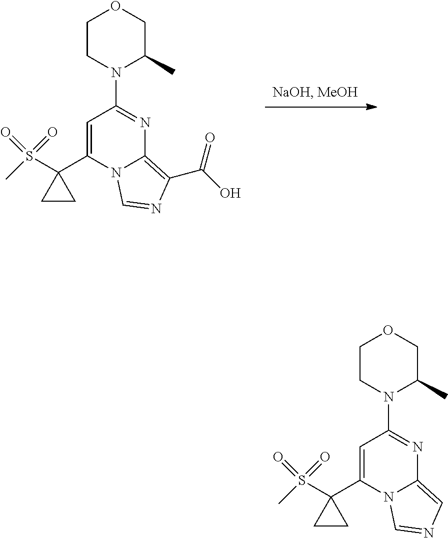

To a solution of (R)-2-(3-methylmorpholino)-4-(1-(methylsulfonyl)cyclopropyl) imidazo[1,5-a]pyrimidine-8-carboxylic acid (298 mg, 0.78 mmol) in co-solvent of MeOH (8 mL) and H₂O (2 mL) was added NaOH (94 mg, 2.35 mmol). The mixture was stirred at 60° C. for 16 h. LC-MS showed the reaction was complete. The mixture was diluted with DCM (40 mL), then washed with water and brine, dried over anhydrous Na₂SO₄, filtered and concentrated. The residue was purified by flash chromatography on silica gel (PE:EA=1:1, V/V) to afford the desired product (130 mg, yield: 49%). LC/MS (ESI): m/z 337 [M+H]⁺.

### Step 9. (R)-4-(8-bromo-4-(1-(methylsulfonyl)cyclopropyl)imidazo[1,5-a]pyrimidin-2-yl)-3-methylmorpholine

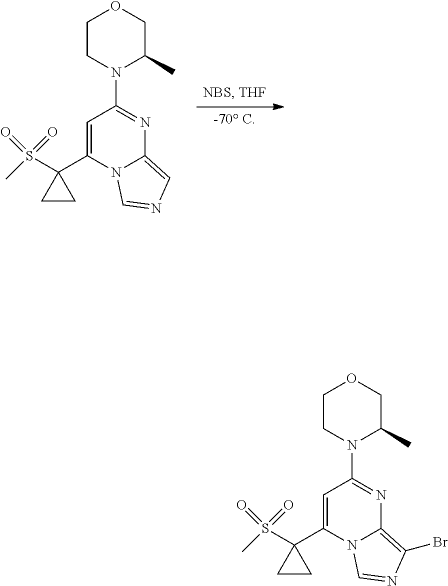

To a solution of (R)-3-methyl-4-(4-(1-(methylsulfonyl)cyclopropyl)imidazo[1,5-a]pyrimidin-2-yl)morpholine (130 mg, 0.386 mmol) in THF (8 mL) at −70° C. was added NBS (69 mg, 0.386 mmol). The mixture was stirred at −70° C. for 30 min. LC-MS showed the reaction was complete. The mixture was quenched with saturated Na₂S₂O₃ aqueous solution, then extracted with DCM (20 mL×3). The combined organic phase was washed brine, dried over anhydrous Na₂SO₄, filtered and concentrated to dryness. The residue was purified by flash chromatography on silica gel (PE:EA=1:1, V/V) to afford the desired product (100 mg, yield: 62%). LC/MS (ESI): m/z 415/417 [M+H]⁺.

### Step 10. (3R)-3-methyl-4-(4-(1-(methylsulfonyl)cyclopropyl)-8-(1-(tetrahydro-2H-pyran-2-yl)-1H-pyrazol-5-yl)imidazo[1,5-a]pyrimidin-2-yl)morpholine

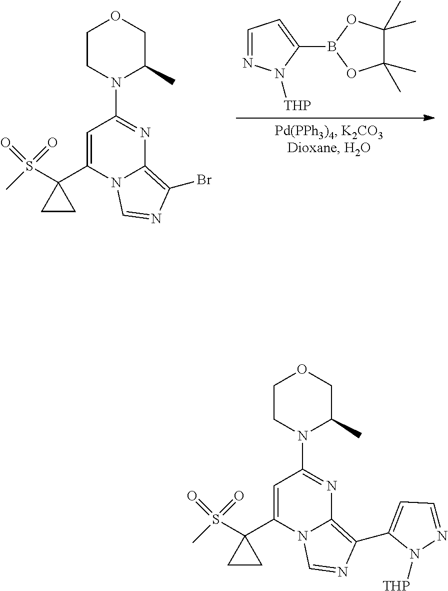

To a solution of (R)-4-(8-bromo-4-(1-(methylsulfonyl)cyclopropyl)imidazo[1,5-a]pyrimidin-2-yl)-3-methylmorpholine (100 mg, 0.24 mmol), 1-(oxan-2-yl)-5-(tetra methyl-1,3,2-dioxaborolan-2-yl)-1H-pyrazole (134 mg, 0.48 mmol) and K₂CO₃ (100 mg, 0.72 mmol) in co-solvent of dioxane (10 mL) and H₂O (2 mL) was added Pd(PPh₃)₄ (56 mg, 0.05 mmol). The mixture was stirred at 100° C. for 15 h under N₂ atmosphere. LC-MS showed the reaction was complete. The mixture was diluted with DCM (40 mL), then washed with water and brine, dried over anhydrous Na₂SO₄, filtered and concentrated. The residue was purified by flash chromatography on silica gel (PE:EA=1:1, V/V) to afford the desired product (37 mg, yield: 32%). LC/MS (ESI): m/z 488 [M+H]⁺.

### Step 11. (R)-3-methyl-4-(4-(1-(methylsulfonyl)cyclopropyl)-8-(1H-pyrazol-5-yl)imidazo[1,5-a]pyrimidin-2-yl)morpholine

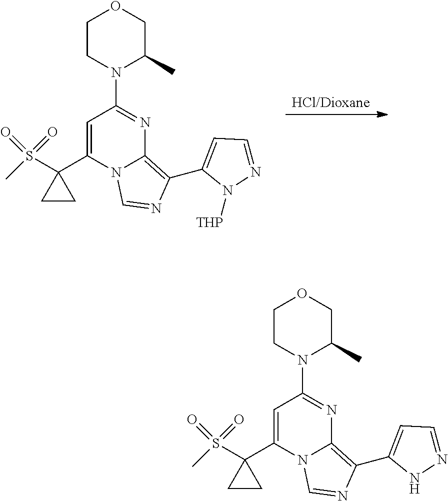

A mixture of (3R)-3-methyl-4-(4-(1-(methylsulfonyl)cyclopropyl)-8-(1-(tetrahydro-2H-pyran-2-yl)-1H-pyrazol-5-yl)imidazo[1,5-a]pyrimidin-2-yl)morpholine (35 mg, 0.07 mmol) in HCl solution (4 M in dioxane, 3 mL) was stirred at room temperature for 1 h. LC-MS showed the reaction was complete. The reaction mixture was concentrated under vacuo. The residue was purified by Prep-HPLC (C₁₈, 10-95%, MeOH in H₂O with 0.1% HCOOH) to give the desired product (6 mg, yield: 21%). LC/MS (ESI): m/z 403 [M+H]⁺. ¹H NMR (400 MHz, DMSO) δ 12.81 (s, 1H), 8.18 (s, 1H), 8.05 (s, 1H), 7.48 (d, J=1.1 Hz, 1H), 7.07 (s, 1H), 6.63 (d, J=1.5 Hz, 1H), 4.54 (d, J=5.3 Hz, 1H), 4.20 (d, J=13.0 Hz, 1H), 3.99 (dd, J=11.4, 3.4 Hz, 1H), 3.78 (d, J=11.4 Hz, 1H), 3.66 (dd, J=11.4, 2.8 Hz, 1H), 3.50 (td, J=11.9, 2.8 Hz, 1H), 3.25 (d, J=9.5 Hz, 1H), 3.20 (s, 3H), 1.26 (d, J=6.7 Hz, 3H).

### Example 25

### Synthesis of (R)-3-methyl-4-(4-(1-methyl-1H-pyrazol-5-yl)-8-(1H-pyrazol-5-yl)imidazo[1,5-a]pyrimidin-2-yl)morpholine

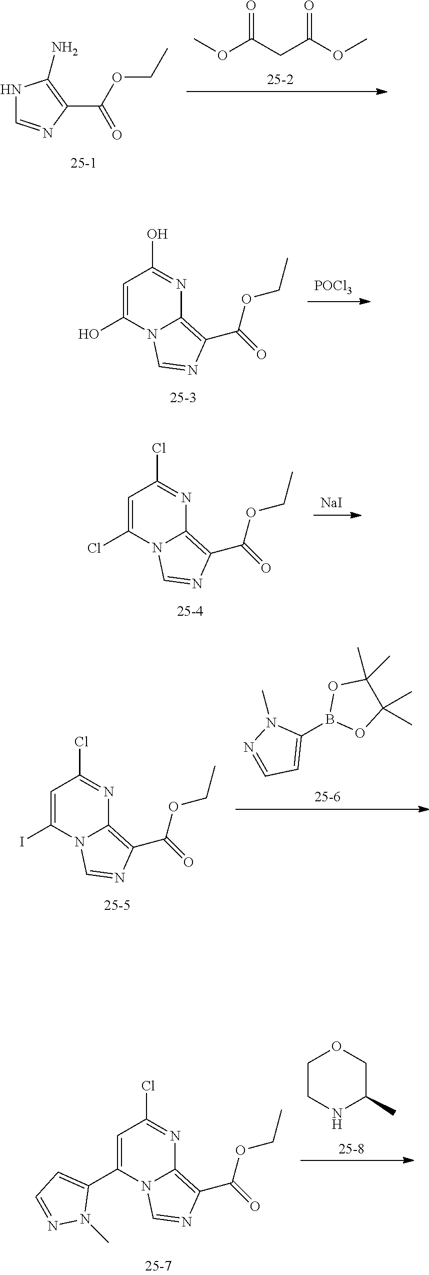

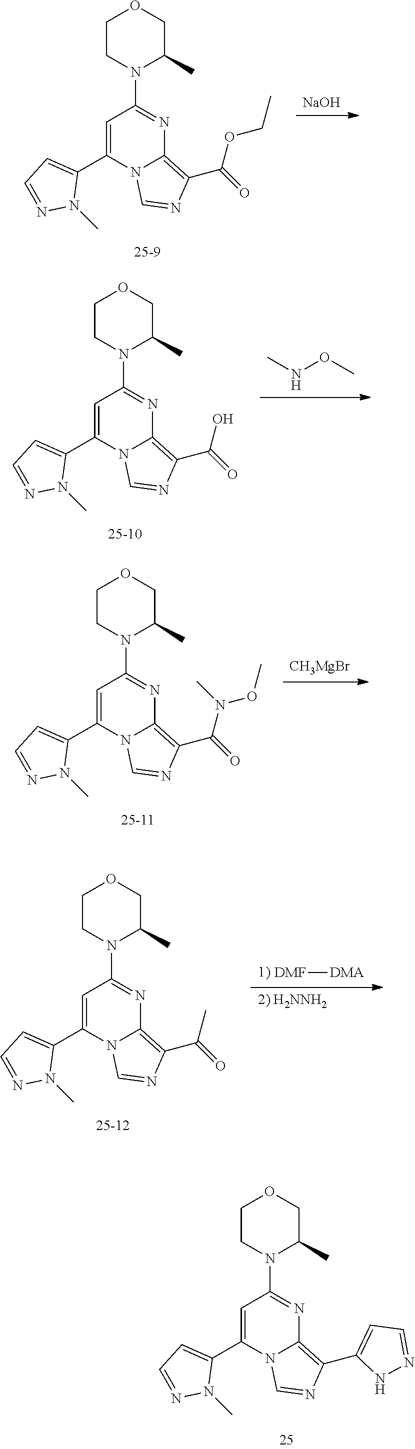

### Step 1. Ethyl 2,4-dihydroxyimidazo[1,5-a]pyrimidine-8-carboxylate

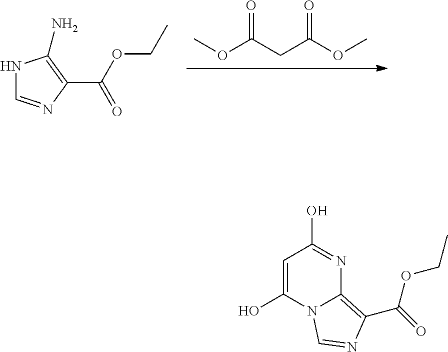

To a suspension of ethyl 5-amino-1H-imidazole-4-carboxylate (2.4 g, 15.47 mmol) and Cs₂CO₃ (15.1 g, 46.40 mmol) in DMF (100 mL) was added 1,3-diethyl propanedioate (4.95 g, 30.94 mmol). The mixture was stirred at 120° C. for 16 h. LC-MS showed the reaction was complete. After cooling to room temperature, the mixture was diluted with DCM (100 mL), then filtered. Then filter cake was washed with DCM and MeOH (4:1, 40 mL). The filtrate was concentrated to give the crude product (3.45 g), which was used in the next step without further purification. LC/MS (ESI): m/z 224 [M+H]⁺.

### Step 2. Ethyl 2,4-dichloroimidazo[1,5-a]pyrimidine-8-carboxylate

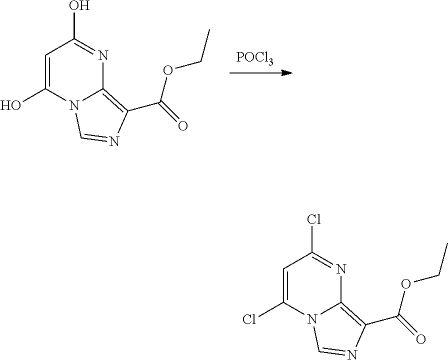

A mixture of ethyl 2,4-dihydroxyimidazo[1,5-a]pyrimidine-8-carboxylate (3.45 g) in POCl₃ (40 mL) was stirred at 100° C. for 2 h. LC-MS showed the reaction was complete. The mixture was concentrated under reduced pressure. The residue was diluted with DCM (100 mL), then washed with saturated NaHCO₃ aqueous solution and brine, dried over anhydrous Na₂SO₄, filtered and concentrated. The residue was purified by flash chromatography on silica gel (PE:EA=5:1, V/V) to give the desired product (1.05 g, yield: 26%). LC/MS (ESI): m/z 260/262 [M+H]⁺.

### Step 3. Ethyl 2-chloro-4-iodoimidazo[1,5-a]pyrimidine-8-carboxylate

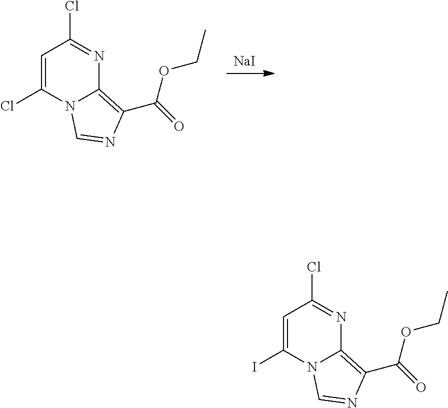

To a solution of ethyl 2,4-dichloroimidazo[1,5-a]pyrimidine-8-carboxylate (1.05 g, 4.04 mmol) in MeCN (30 mL) was added NaI (3.03 g, 20.19 mmol). The mixture was stirred at 80° C. for 8 h. LC-MS showed the reaction was complete. The mixture was diluted with EA (60 mL), then washed with saturated Na₂S₂O₃ aqueous solution and brine, dried over anhydrous Na₂SO₄, filtered and concentrated. The residue was purified by column chromatography on silica gel (PE:EA=5:1, V/V) to afford the desired product (1.4 g, yield: 98%). LC/MS (ESI): m/z 352 [M+H]⁺.

### Step 4. Ethyl 2-chloro-4-(1-methyl-1H-pyrazol-5-yl)imidazo[1,5-a]pyrimidine-8-carboxylate

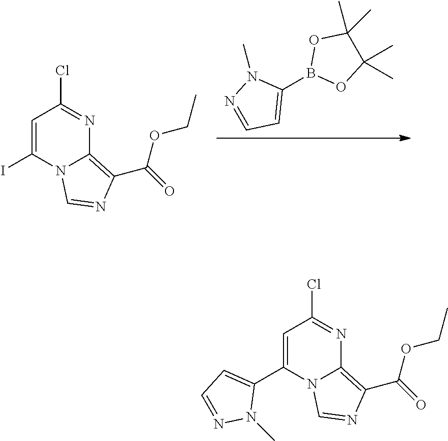

To a solution of ethyl 2-chloro-4-iodoimidazo[1,5-a]pyrimidine-8-carboxylate (1.4 g, 3.98 mmol), 1-methyl-5-(tetramethyl-1,3,2-dioxaborolan-2-yl)-1H-pyrazole (1.24 g, 5.97 mmol) and Na₂CO₃ (2M in H₂O, 6 mL, 11.95 mmol) in DME (30 mL) was added Pd(PPh₃)₄ (0.23 g, 0.199 mmol). The mixture was stirred at 40° C. for 16 h under N₂ atmosphere. LC-MS showed the reaction was complete. The mixture was diluted with EA (60 mL), then washed with water and brine, dried over anhydrous Na₂SO₄, filtered and concentrated. The residue was purified by column chromatography on silica gel (PE:EA=3:1, V/V) to afford the desired product (415 mg, yield: 34%). LC/MS (ESI): m/z 306 [M+H]⁺.

### Step 5. Ethyl (R)-4-(1-methyl-1H-pyrazol-5-yl)-2-(3-methylmorpholino)imidazo[1,5-a]pyrimidine-8-carboxylate

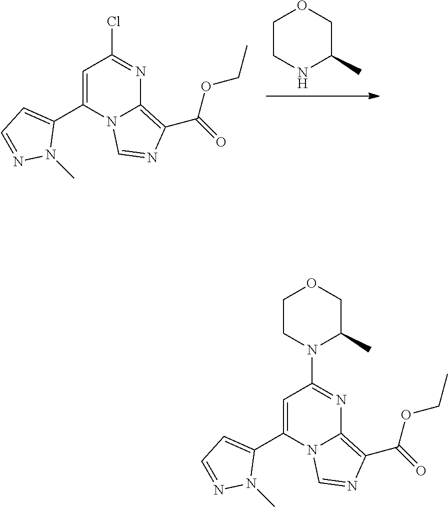

To a solution of ethyl 2-chloro-4-(1-methyl-1H-pyrazol-5-yl)imidazo[1,5-a]pyrimidine-8-carboxylate (415 mg, 1.36 mmol) in MeCN (10 mL) was added (3R)-3-methylmorpholine (412 mg, 4.07 mmol). The mixture was stirred at 80° C. for 16 h. LC-MS showed the reaction was complete. The mixture was concentrated to dryness. The residue was purified by flash chromatography on silica gel (PE:EA=1:1, V/V) to afford the desired product (447 mg, yield: 89%). LC/MS (ESI): m/z 371 [M+H]⁺.

### Step 6. (R)-4-(1-methyl-1H-pyrazol-5-yl)-2-(3-methylmorpholino)imidazo[1,5-a]pyrimidine-8-carboxylic acid

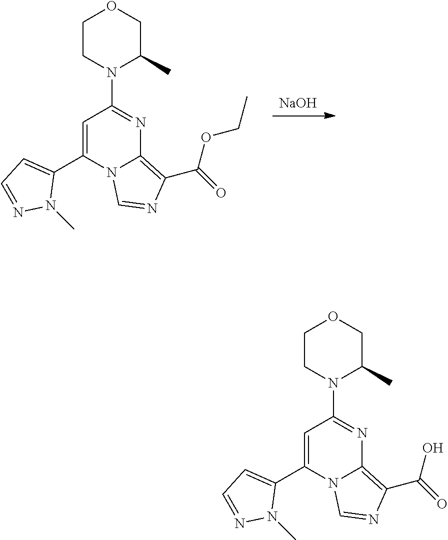

To a solution of ethyl (R)-4-(1-methyl-1H-pyrazol-5-yl)-2-(3-methylmorpholino) imidazo[1,5-a]pyrimidine-8-carboxylate (447 mg, 1.21 mmol) in co-solvent of MeOH (9 mL) and H₂O (3 mL) was added NaOH (145 mg, 3.62 mmol). The mixture was stirred at 50° C. for 16 h. LC-MS showed the reaction was complete. The reaction mixture was adjusted to PH=5 by the addition of HCl solution (1N), then extracted with DCM (30 mL×3). The combined organic layer was washed with brine, dried over anhydrous Na₂SO₄, filtered and concentrated to give the desired product (387 mg, yield: 94%). LC/MS (ESI): m/z 343 [M+H]⁺.

### Step 7. (R)—N-methoxy-N-methyl-4-(1-methyl-1H-pyrazol-5-yl)-2-(3-methylmorpholino)imidazo[1,5-a]pyrimidine-8-carboxamide

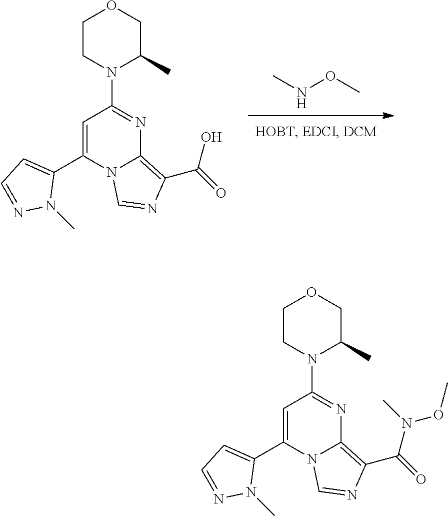

To a solution of (R)-4-(1-methyl-1H-pyrazol-5-yl)-2-(3-methylmorpholino)imidazo [1,5-a]pyrimidine-8-carboxylic acid (380 mg, 1.11 mmol), HOBT (225 mg, 1.67 mmol), EDCI (319 mg, 1.66 mmol) and TEA (0.62 mL, 4.44 mmol) in DCM (10 mL) was added methoxy(methyl)amine (0.111 mL, 1.44 mmol).

The mixture was stirred at room temperature for 3 h. LC-MS showed the reaction was complete. The mixture was diluted with EA (60 mL), then washed with water and brine, dried over anhydrous Na₂SO₄, filtered and concentrated. The residue was purified by column chromatography on silica gel (PE:EA=3:1, V/V) to afford the desired product (427 mg, yield: 99%). LC/MS (ESI): m/z 386 [M+H]⁺.

### Step 8. (R)-1-(4-(1-methyl-1H-pyrazol-5-yl)-2-(3-methylmorpholino)imidazo[1,5-a]pyrimidin-8-yl)ethan-1-one

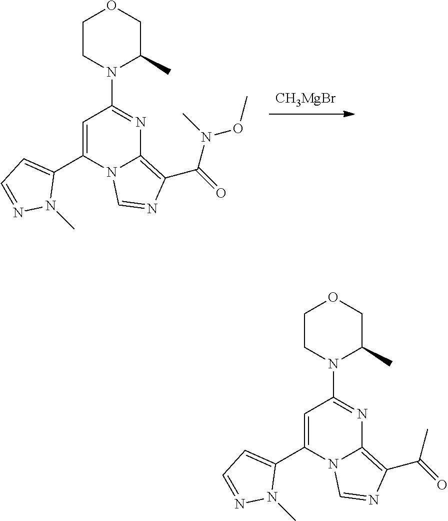

To a solution of (R)—N-methoxy-N-methyl-4-(1-methyl-1H-pyrazol-5-yl)-2-(3-methyl morpholino)imidazo[1,5-a]pyrimidine-8-carboxamide (427 mg, 1.11 mmol) in THF (10 mL) at 0° C. was added CH₃MgBr (2.5 M, 0.9 mL, 2.22 mmol) drop wise.

The mixture was stirred at 0° C. for 2 h. LC-MS showed the reaction was complete.

The mixture was quenched with saturated NH₄Cl aqueous solution, then extracted with EA (30 mL×3). The combined organic phase was washed with brine, dried over anhydrous Na₂SO₄, filtered and concentrated to dryness. The residue was purified by flash chromatography on silica gel (PE:EA=1:1, V/V) to afford the desired product (335 mg, yield: 89%). LC/MS (ESI): m/z 341 [M+H]⁺.

### Step 9. (R)-3-methyl-4-(4-(1-methyl-1H-pyrazol-5-yl)-8-(1H-pyrazol-5-yl)imidazo[1,5-a]pyrimidin-2-yl)morpholine

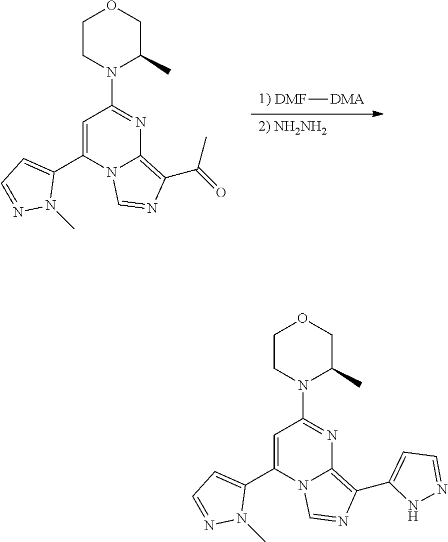

A mixture of (R)-1-(4-(1-methyl-1H-pyrazol-5-yl)-2-(3-methylmorpholino)imidazo [1,5-a]pyrimidin-8-yl)ethan-1-one (150 mg, 0.441 mmol) in DMF-DMA (3 mL, 22.41 mmol) was stirred at 120° C. for 48 h. LC-MS showed the reaction was complete. The mixture was concentrated to obtain a yellow oil (180 mg), which was used in the next step without further purification. The yellow oil was dissolved in EtOH (3 mL), then hydrazine hydrate (1 mL) was added. The mixture was stirred at 75° C. for 2 h. LC-MS showed the reaction was complete.

The reaction mixture was diluted with EA (60 mL), then washed with water and brine, dried over anhydrous Na₂SO₄, filtered and concentrated. The residue was purified by Prep-HPLC (C₁₈, 10-95%, MeOH in H₂O with 0.1% HCOOH) to give the desired product (11 mg, yield: 7%). LC/MS (ESI): m/z 365 [M+H]⁺. ¹H NMR (400 MHz, DMSO) δ 7.86 (s, 1H), 7.73 (d, J=2.0 Hz, 1H), 7.54 (s, 1H), 6.97-6.90 (m, 2H), 6.70 (d, J=1.2 Hz, 1H), 4.57 (dd, J=14.3, 6.9 Hz, 1H), 4.24 (d, J=13.2 Hz, 1H), 3.99 (dd, J=11.6, 3.3 Hz, 1H), 3.95 (s, 3H), 3.77 (d, J=11.4 Hz, 1H), 3.68-3.64 (m, 1H), 3.52-3.49 (m, 1H), 3.26-3.23 (m, 1H), 1.27 (d, J=6.7 Hz, 3H).

### Example 27

### Synthesis of (R)-3-methyl-4-(4-(1-methyl-1H-pyrazol-5-yl)-7-(1H-pyrazol-5-yl)imidazo[1,5-b]pyridazin-2-yl)morpholine

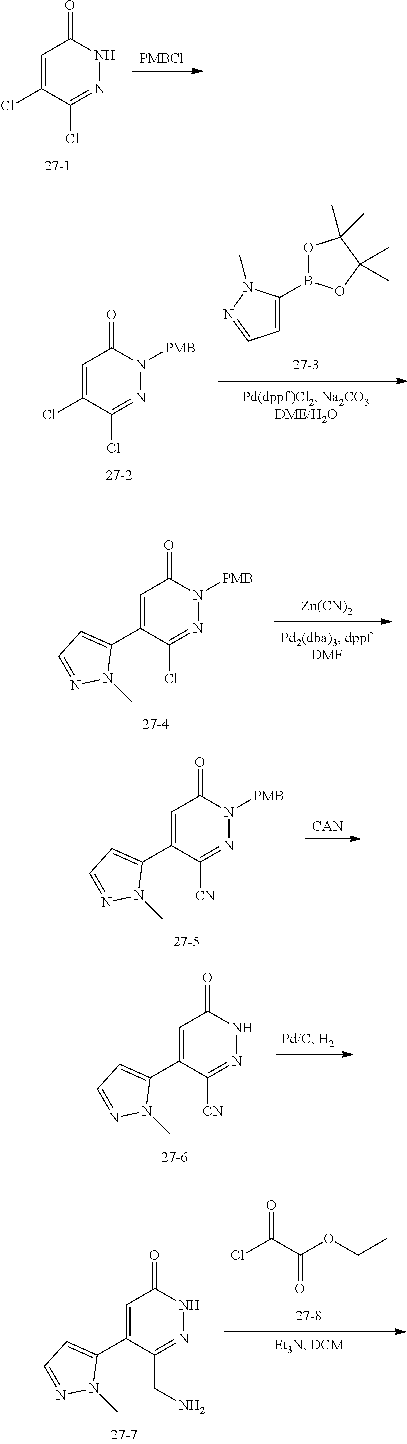

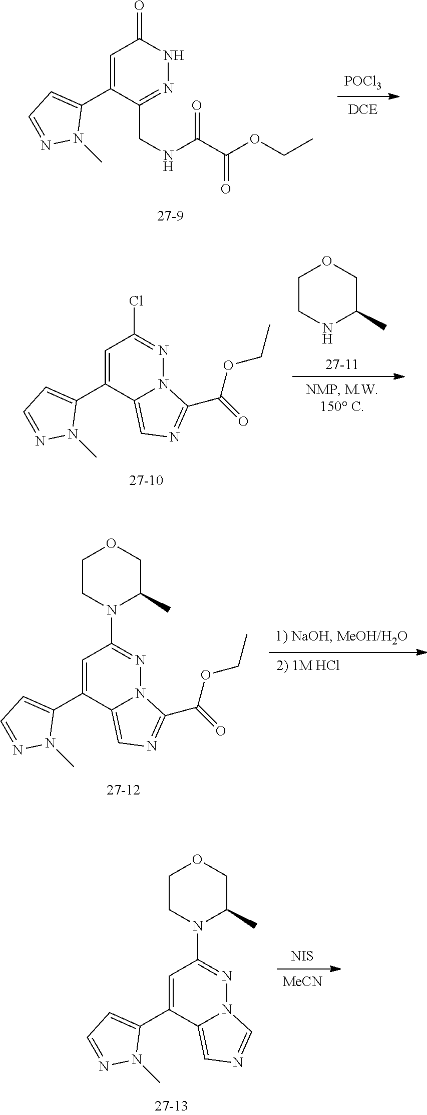

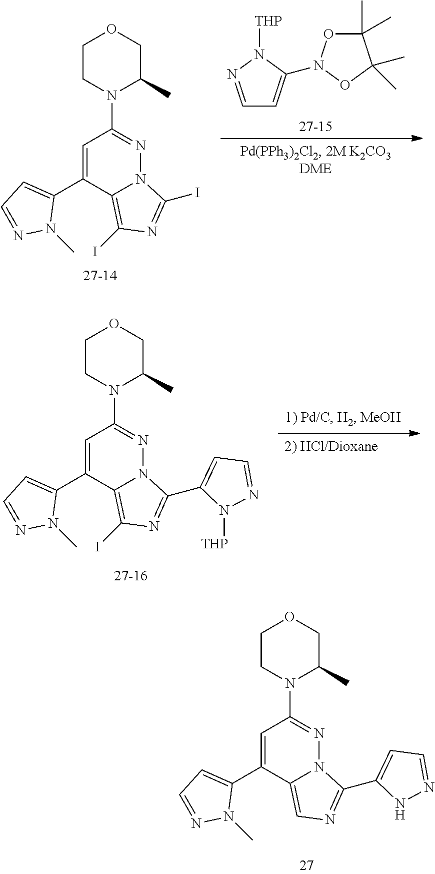

### Step 1. 5,6-dichloro-2-(4-methoxybenzyl)pyridazin-3 (2H)-one

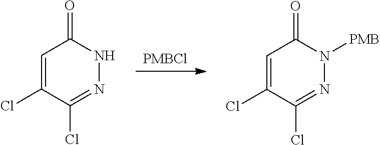

To a solution of 5,6-dichloropyridazin-3 (2H)-one (300 mg, 1.82 mmol) in DMF (5 mL) was added K₂CO₃ (754.0 mg, 5.46 mmol) and 1-(chloromethyl)-4-methoxy benzene (0.50 mL, 3.64 mmol). The reaction was stirred at room temperature overnight. LC-MS showed the reaction was complete. The mixture was diluted with EA (40 mL), then washed with water and brine, dried over anhydrous Na₂SO₄, filtered and concentrated. The residue was purified by column chromatography on silica gel (PE:EA=20:1, V/V) to afford the desired product (400 mg, yield: 77%). LC/MS (ESI): m/z 285 [M+H]⁺. ¹H NMR (400 MHz, CDCl₃) δ 7.41-7.36 (m, 1H), 7.07 (s, 1H), 6.88-6.83 (m, 1H), 5.18 (s, 1H), 3.79 (s, 2H).

### Step 2. 6-chloro-2-(4-methoxybenzyl)-5-(1-methyl-1H-pyrazol-5-yl)pyridazin-3 (2H)-one

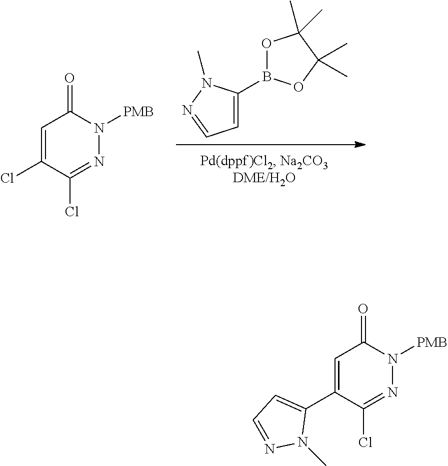

To a solution of 5,6-dichloro-2-(4-methoxybenzyl)pyridazin-3 (2H)-one (200 mg, 0.70 mmol) and 1-methyl-5-(4,4,5,5-tetramethyl-1,3,2-dioxaborolan-2-yl)-1H-pyrazole (291.9 mg, 1.40 mmol) in DME (10 mL) were added Na₂CO₃ (2M in H₂O, 0.88 mL, 1.75 mmol) and Pd(dppf)Cl₂ (51.3 mg, 0.07 mmol). The mixture was stirred at 100° C. overnight under nitrogen atmosphere. LC-MS showed the reaction was complete. The mixture was diluted with EA (40 mL), then washed with water and brine, dried over anhydrous Na₂SO₄, filtered and concentrated. The residue was purified by column chromatography on silica gel (PE:EA=1:1, V/V) to afford the desired product (110 mg, yield: 47%). LC/MS (ESI) m/z: 331 [M+H]. ¹H NMR (400 MHz, CDCl₃) δ 7.60 (d, J=1.7 Hz, 1H), 7.47 (d, J=8.7 Hz, 2H), 6.95-6.86 (m, 3H), 6.41 (d, J=1.7 Hz, 1H), 5.25 (s, 2H), 3.84 (s, 3H), 3.80 (s, 3H).

### Step 3. 1-(4-methoxybenzyl)-4-(1-methyl-1H-pyrazol-5-yl)-6-oxo-1,6-dihydropyridazine-3-carbonitrile

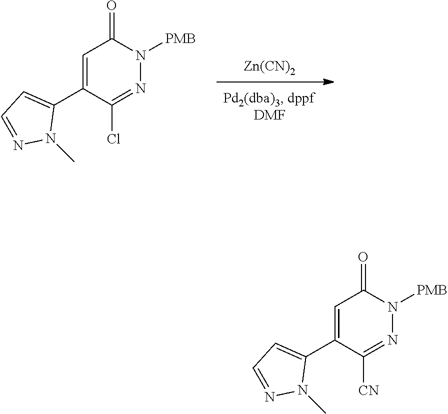

To a solution of 6-chloro-2-(4-methoxybenzyl)-5-(1-methyl-1H-pyrazol-5-yl) pyridazin-3 (2H)-one (450 mg, 1.36 mmol) in DMF (8 mL) were added Zn(CN)₂ (319.6 mg, 2.72 mmol), dppf (150.8 mg, 0.27 mmol) and Pd₂(dba)₃ (124.6 mg, 0.14 mmol). The reaction was stirred at 120° C. overnight under nitrogen atmosphere. LC-MS showed the reaction was complete. The mixture was diluted with EA (40 mL), then washed with water and brine, dried over anhydrous Na₂SO₄, filtered and concentrated. The residue was purified by column chromatography on silica gel (PE:EA=2:1, V/V) to afford the desired product (200 mg, yield: 46%). LC/MS (ESI) m/z: 322 [M+H]⁺.

### Step 4. 4-(1-methyl-1H-pyrazol-5-yl)-6-oxo-1,6-dihydropyridazine-3-carbonitrile

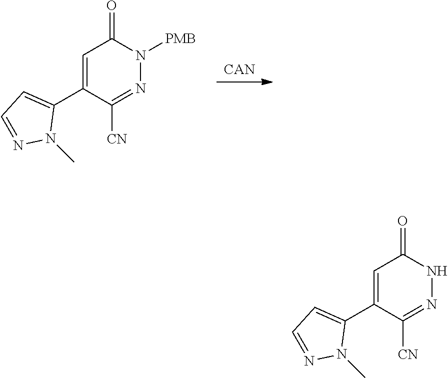

To a solution of 1-(4-methoxybenzyl)-4-(1-methyl-1H-pyrazol-5-yl)-6-oxo-1,6-dihydropyridazine-3-carbonitrile (660 mg, 2.05 mmol) in CH₃CN (30 mL) and H₂O (6 mL) was added Ceric ammonium nitrate (4.1 mL, 8.22 mmol). The mixture was stirred at room temperature overnight. LC-MS showed the reaction was complete. The mixture was diluted with DCM (40 mL), then washed with water and brine, dried over anhydrous Na₂SO₄, filtered and concentrated. The residue was purified by column chromatography on silica gel (PE:EA=1:1, V/V) to afford the desired product (350 mg, yield: 85%). LC/MS (ESI) m/z: 202 [M+H]⁺.

### Step 5. 6-(aminomethyl)-5-(1-methyl-1H-pyrazol-5-yl)pyridazin-3 (2H)-one

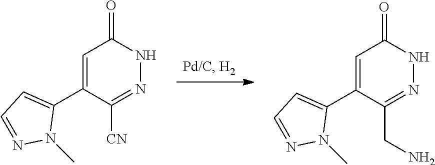

To a solution of 4-(1-methyl-1H-pyrazol-5-yl)-6-oxo-1,6-dihydro pyridazine-3-carbonitrile (350 mg, 1.74 mmol) in MeOH (20 mL) was added Pd/C (10%, 35 mg) and one drop of conc. HCl. The mixture was stirred at room temperature for 4 h under H₂ atmosphere. LC-MS showed the reaction was complete. The reaction mixture was filtered and the filtrate was concentrated to give the desired product (350 mg, yield: 98%). LC/MS (ESI) (m/z): 206 [M+H]⁺.

### Step 6. ethyl 2-(((4-(1-methyl-1H-pyrazol-5-yl)-6-oxo-1,6-dihydropyridazin-3-yl)methyl)amino)-2-oxoacetate

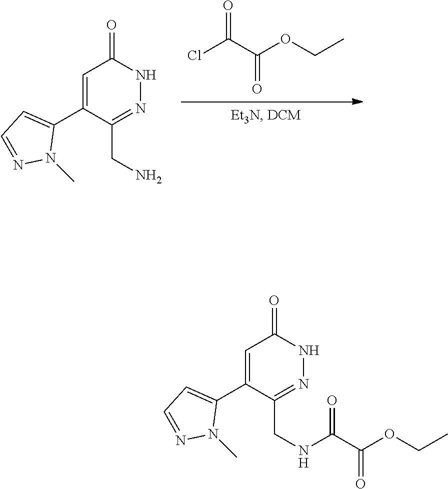

To a solution of 6-(aminomethyl)-5-(1-methyl-1H-pyrazol-5-yl)pyridazin-3 (2H)-one (350 mg, 1.71 mmol) and TEA (0.95 mL, 6.82 mmol) in DCM (30 mL) was added ethyl 2-chloro-2-oxoacetate (0.286 mL, 2.558 mmol). The mixture was stirred at room temperature for 3 h. LC-MS showed the reaction was complete. The mixture was diluted with DCM (40 mL), then washed with water and brine, dried over anhydrous Na₂SO₄, filtered and concentrated. The residue was purified by column chromatography on silica gel (DCM:MeOH=30:1, V/V) to afford the desired product (400 mg, yield: 77%). LC/MS (ESI) m/z: 306 [M+H]⁺.

### Step 7. ethyl 2-chloro-4-(1-methyl-1H-pyrazol-5-yl)imidazo[1,5-b]pyridazine-7-carboxylate

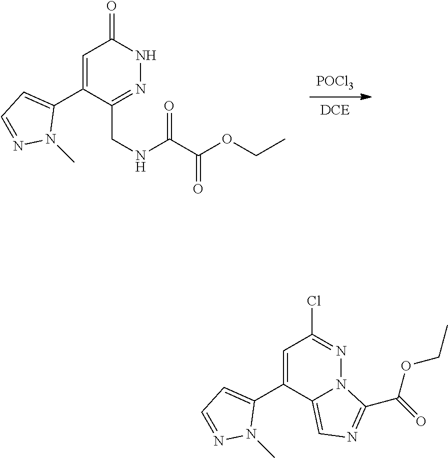 [SMILES: CCOC(=O)C(=O)NCc1n[nH]c(=O)cc1-c1ccnn1C ; "POCl3" ; "DCE" ; CCOC(=O)c1ncc2c(-c3ccnn3C)cc(Cl)nn12]

To a solution of ethyl 2-(((4-(1-methyl-1H-pyrazol-5-yl)-6-oxo-1,6-dihydropyridazin-3-yl)methyl)amino)-2-oxoacetate (250 mg, 0.82 mmol) in 1,2-dichloroethane (5 mL) was added POCl₃ (0.46 mL, 4.91 mmol) dropwise. The mixture was stirred at 80° C. overnight. LC-MS showed the reaction was complete. The reaction mixture was concentrated under vacuo. The residue was diluted with DCM (40 mL), then washed with saturated NaHCO₃ aqueous solution and brine, dried over anhydrous Na₂SO₄, filtered and concentrated. The residue was purified by column chromatography on silica gel (DCM:MeOH=50:1, V/V) to afford the desired product (200 mg, yield: 79%). LC/MS (ESI) m/z: 306 [M+H]⁺. ¹H NMR (400 MHz, DMSO) δ 7.81 (s, 1H), 7.71 (d, J=2.0 Hz, 1H), 7.46 (s, 1H), 6.93 (d, J=2.0 Hz, 1H), 4.41 (q, J=7.1 Hz, 2H), 4.00 (s, 3H), 1.36 (t, J=7.1 Hz, 3H).

### Step 8. ethyl (R)-4-(1-methyl-1H-pyrazol-5-yl)-2-(3-methylmorpholino)imidazo[1,5-b]pyridazine-7-carboxylate

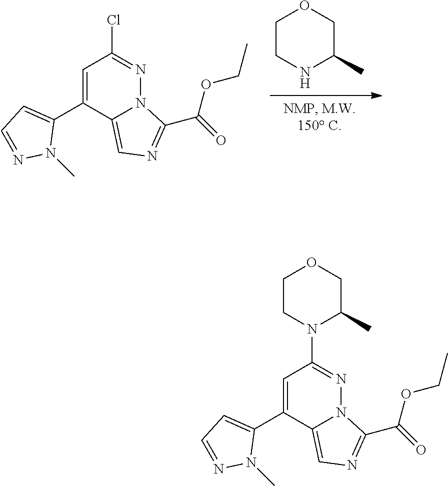

To a solution of ethyl 2-chloro-4-(1-methyl-1H-pyrazol-5-yl)imidazo[1,5-b]pyridazine-7-carboxylate (200 mg, 0.65 mmol) in NMP (10 mL) was added (3R)-3-methylmorpholine (264.7 mg, 2.62 mmol). The mixture was stirred at 150° C. for 1 h under microwave irradiation. LC-MS showed the reaction was complete. The mixture was diluted with DCM (40 mL), then washed with water and brine, dried over anhydrous Na₂SO₄, filtered and concentrated. The residue was purified by column chromatography on silica gel (DCM:MeOH=20:1, V/V) to afford the desired product (80 mg, yield: 33%). LC/MS (ESI) m/z: 371 [M+H]⁺.

### Step 9. (R)-3-methyl-4-(4-(1-methyl-1H-pyrazol-5-yl)imidazo[1,5-b]pyridazin-2-yl)morpholine

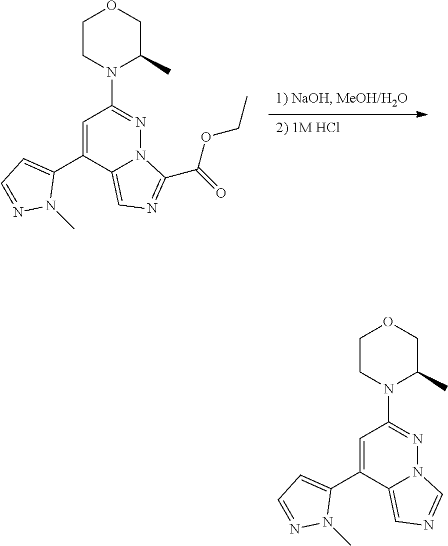

To a solution of ethyl (R)-4-(1-methyl-1H-pyrazol-5-yl)-2-(3-methylmorpholino) imidazo[1,5-b]pyridazine-7-carboxylate (200 mg, 0.54 mmol) in co-solvent of MeOH (3 mL) and H₂O (1 mL) was added NaOH (64.8 mg, 1.62 mmol). The mixture was stirred at 50° C. for 1 h. After cooling to room temperature, the reaction mixture was adjusted to pH=3 by the addition of HCl solution (1 M), then extracted with EA (20 mL×3). The combined organic layer was washed with brine, dried over anhydrous Na₂SO₄, filtered and concentrated. The residue was purified by column chromatography on silica gel (DCM:MeOH=30:1, V/V) to afford the desired product (100 mg, yield: 62%). LC/MS (ESI) m/z: 299 [M+H]⁺.

### Step 10. (R)-4-(5,7-diiodo-4-(1-methyl-1H-pyrazol-5-yl)imidazo[1,5-b]pyridazin-2-yl)-3-methylmorpholine

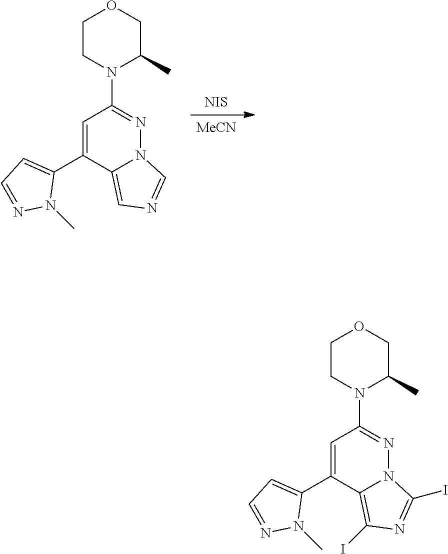

To a solution of (R)-3-methyl-4-(4-(1-methyl-1H-pyrazol-5-yl)imidazo[1,5-b]pyridazin-2-yl)morpholine (60 mg, 0.20 mmol) in CH₃CN (2 mL) was added NIS (135.7 mg, 0.60 mmol). The mixture was stirred at room temperature for 2 h. LC-MS showed the reaction was complete. The mixture was diluted with DCM (40 mL), then washed with water and brine, dried over anhydrous Na₂SO₄, filtered and concentrated. The residue was purified by column chromatography on silica gel (PE:EA=1:1, V/V) to afford the desired product (100 mg, yield: 90%). LC/MS (ESI) m/z: 551 [M+H]⁺.

### Step 11. (3R)-4-(5-iodo-4-(1-methyl-1H-pyrazol-5-yl)-7-(1-(tetrahydro-2H-pyran-2-yl)-1H-pyrazol-5-yl)imidazo[1,5-b]pyridazin-2-yl)-3-methylmorpholine

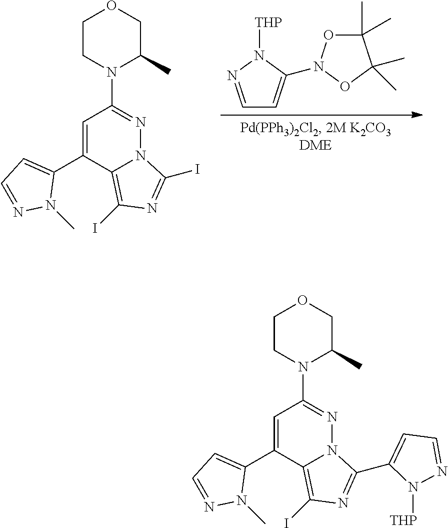

To a solution of (R)-4-(5,7-diiodo-4-(1-methyl-1H-pyrazol-5-yl)imidazo[1,5-b] pyridazin-2-yl)-3-methylmorpholine (100 mg, 0.18 mmol) and 1-(tetrahydro-2H-pyran-2-yl)-5-(4,4,5,5-tetramethyl-1,3,2-dioxaborolan-2-yl)-1H-pyrazole (75.8 mg, 0.27 mmol) in DME (5 mL) were added K₂CO₃ (2M in H₂O, 0.27 mL, 0.55 mmol) and Bis(triphenylphosphine)palladium(II) chloride (141.4 mg, 0.18 mmol). The mixture was stirred at 80° C. overnight under nitrogen atmosphere. LC-MS showed the reaction was complete. The mixture was diluted with EA (40 mL), then washed with water and brine, dried over anhydrous Na₂SO₄, filtered and concentrated. The residue was purified by column chromatography on silica gel (DCM:MeOH=30:1, V/V) to afford the desired product (60 mg, yield: 57%). LC/MS (ESI) m/z: 575 [M+H]⁺.

### Step 12. (R)-3-methyl-4-(4-(1-methyl-1H-pyrazol-5-yl)-7-(1H-pyrazol-5-yl)imidazo[1,5-b]pyridazin-2-yl)morpholine

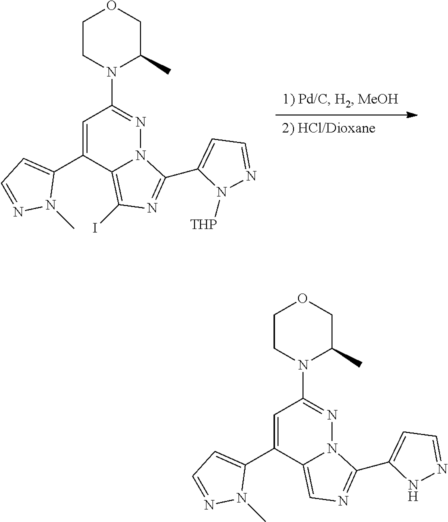

To a solution of (3R)-4-(5-iodo-4-(1-methyl-1H-pyrazol-5-yl)-7-(1-(tetrahydro-2H-pyran-2-yl)-1H-pyrazol-5-yl)imidazo[1,5-b]pyridazin-2-yl)-3-methylmorpholine (50 mg, 0.09 mmol) in MeOH (5 mL) was added Pd/C (10%, 10 mg). The mixture was stirred at room temperature for 2 h under N₂ atmosphere. LC-MS showed the reaction was complete. The mixture was filtered and the filtrate was concentrated to dryness. The residue was dissolved in DCM (2 mL), then HCl solution (4M in dioxane, 1 mL) was added. The mixture was stirred at room temperature for 2 h. LC-MS showed the reaction was complete. The reaction mixture was concentrated under vacuo. The residue was purified by Pre-HPLC (Cis, 10-95%, MeOH in H₂O with 0.1% HCOOH) to afford the desired product (3 mg, yield: 9%). LC/MS (ESI) m/z: 365 [M+H]⁺. ¹H NMR (400 MHz, DMSO) δ 7.73 (s, 1H), 7.66 (d, J=1.9 Hz, 1H), 7.43 (s, 1H), 7.14 (d, J=1.9 Hz, 1H), 6.98 (s, 1H), 6.81 (d, J=1.9 Hz, 1H), 4.40 (d, J=6.5 Hz, 1H), 4.01 (dd, J=11.6, 3.5 Hz, 1H), 3.98 (s, 3H), 3.95 (s, 1H), 3.91 (s, 1H), 3.78 (d, J=11.3 Hz, 1H), 3.73 (dd, J=11.4, 2.7 Hz, 1H), 3.61-3.54 (m, 1H), 3.28 (dd, J=12.7, 3.7 Hz, 2H), 1.26 (d, J=6.7 Hz, 3H).

The title compound can also been synthesized following the procedure as shown below.

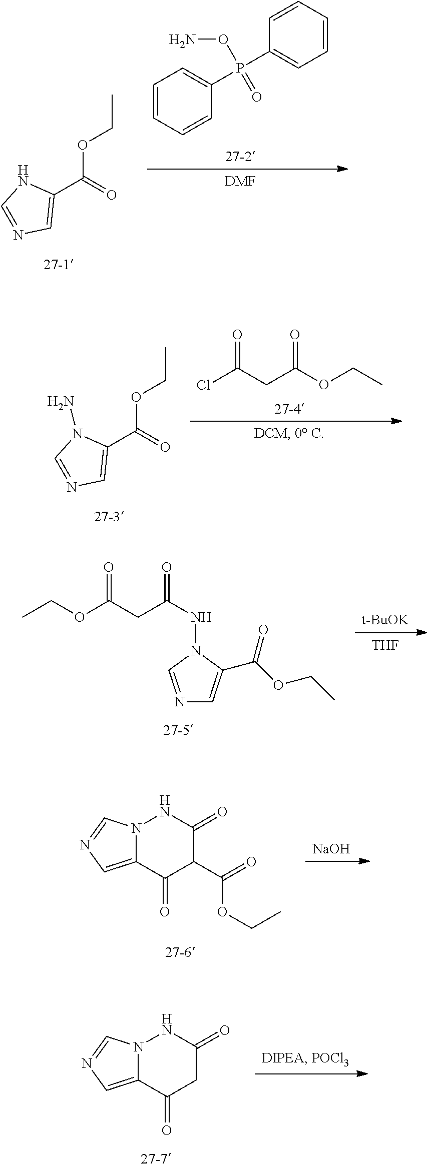

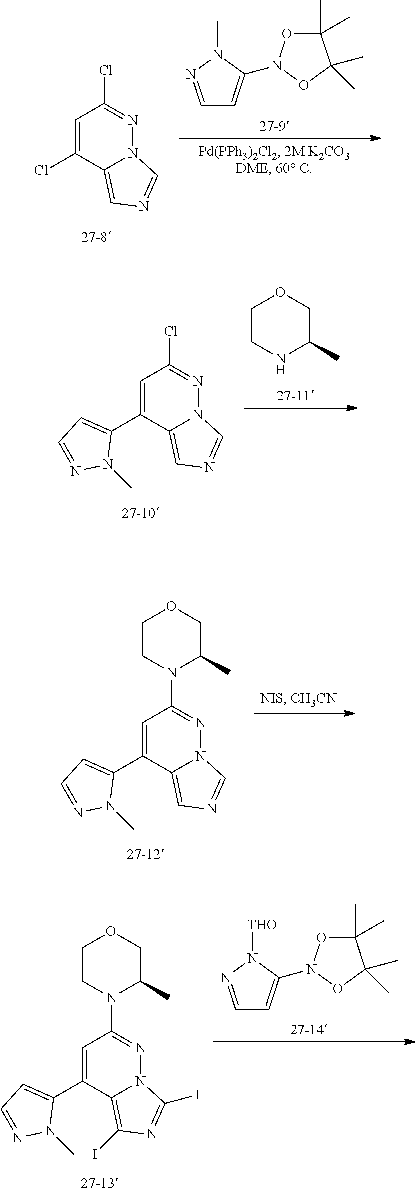

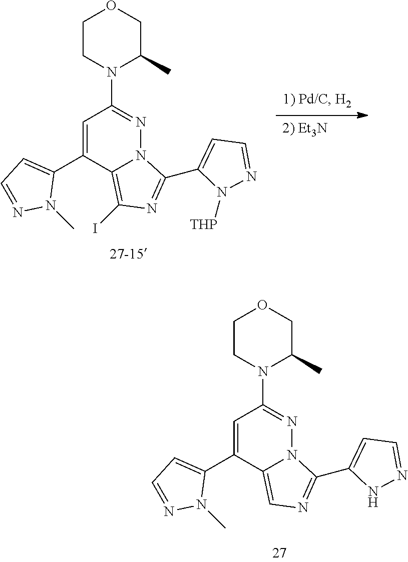

### Step 1. ethyl 1-amino-1H-imidazole-5-carboxylate

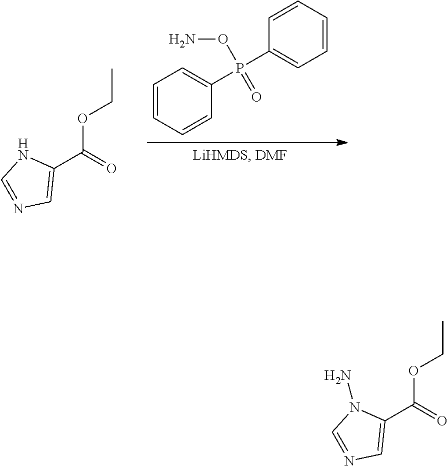

To a solution of ethyl 1H-imidazole-5-carboxylate (2 g, 178 mmol) in DMF (200 mmol) at 0° C. LiHMDS (1M in THF, 196 mL, 196 mmol) dropwise. The mixture was stirred at 0° C. for 1 h, them amino diphenylphosphinate (50 g, 214 mmol) was added portion wise. After the addition, the resulting mixture was stirred at 0° C. for an additional 2 h. LC-MS showed the reaction was complete. The reaction mixture was quenched with H₂O (200 mL), then concentrated to dryness. The residue was diluted with EA (500 mL), then filtered. The filter cake was washed with EA (200 mL). The combined organic phase was dried over anhydrous Na₂SO₄, filtered and concentrated. The residue was purified by column chromatography (DCM:MeOH=10:1, V/V) to give the desired product (14 g, yield: 50.6%). LC/MS (ESI): m/z 156.2 [M+H]⁺.

### Step 2. ethyl 1-(3-ethoxy-3-oxopropanamido)-1H-imidazole-5-carboxylate

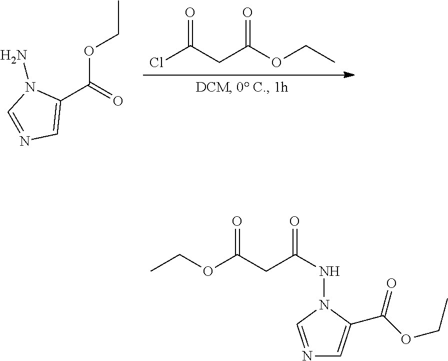

To a solution of ethyl 1-amino-1H-imidazole-5-carboxylate (14 g, 90.2 mmol) in DCM (200 mL) at 0° C. was added ethyl 3-chloro-3-oxopropanoate (15.1 mL, 117 mmol) drop wise. The mixture was stirred at room temperature for 16 h. LC-MS showed the reaction was complete. The reaction mixture was quenched with saturated NaHCO₃ aqueous solution, then extracted with DCM (100 mL×3). The combined organic phase was washed with brine, dried over anhydrous Na₂SO₄, filtered and concentrated to dryness. The residue was purified by column chromatography (DCM:MeOH=10:1, V/V) to give the desired product (24 g, yield: 98%). LC/MS (ESI): m/z 270.3 [M+H]⁺.

### Step 3. ethyl 2-hydroxy-4-oxo-3,4-dihydroimidazo[1,5-b]pyridazine-3-carboxylate

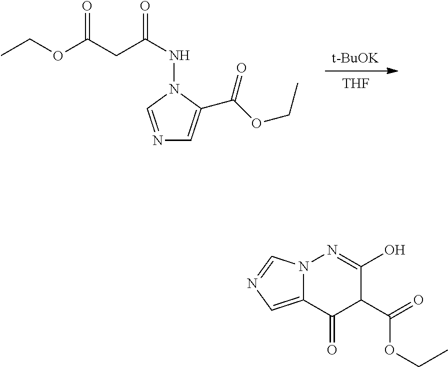 [SMILES: "t-BuOK" ; CCOC(=O)CC(=O)Nn1cncc1C(=O)OCC ; "THF" ; CCOC(=O)C1C(=O)c2cncn2N=C1O]

To a suspension of ethyl 1-(3-ethoxy-3-oxopropanamido)-1H-imidazole-5-carboxylate (24 g, 89.1 mmol) in THF (300 mL) at 0° C. was added t-BuOK (30 g, 267.0 mmol) portion wise. After the addition, the mixture was stirred at room temperature for 5 h. LC-MS showed the reaction was complete. The reaction mixture was adjusted to PH=2 by the addition of 6M HCl aqueous solution, then concentrated to dryness. The residue was suspended in co-solvent of DCM and MeOH (2:1, V:V, 200 mL), then stirred at room temperature for 0.5 h. The resulting mixture was filtered, the filter cake was washed with DCM and MeOH (2:1, V/V, 100 mL). The filtrate was concentrated under reduced pressure to give the crude product, which was used in the next step without further purification (16 g). LC/MS (ESI): m/z 224.2 [M+H]⁺.

### Step 4. imidazo[1,5-b]pyridazine-2,4 (1H,3H)-dione

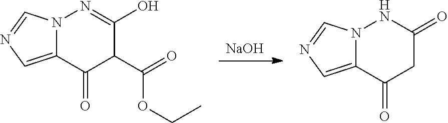 [SMILES: CCOC(=O)C1C(=O)c2cncn2N=C1O ; "NaOH" ; O=C1CC(=O)c2cncn2N1]

A mixture of ethyl 2-hydroxy-4-oxo-3,4-dihydroimidazo[1,5-b]pyridazine-3-carboxylate (16 g, 71.7 mmol) in NaOH aqueous solution (4M, 120 mL) was stirred at 100° C. for 16 h. LC-MS showed the reaction was complete. After cooling to room temperature, the mixture was adjusted to PH=2 by the addition of 6M HCl aqueous solution, then filtered. The filter cake was washed with ice-water twice (50 mL×2), then concentrated under vacuo to give the desired product (8 g, yield: 59%).

LC/MS (ESI): m/z 152 [M+H]⁺.

### Step 5. 2,4-dichloroimidazo[1,5-b]pyridazine

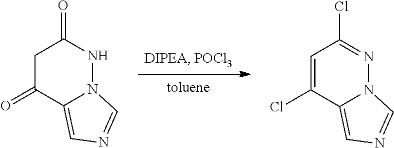 [SMILES: O=C1CC(=O)c2cncn2N1 ; "DIPEA, POCl3" ; "toluene" ; Clc1cc(Cl)c2cncn2n1]

To a solution of imidazo[1,5-b]pyridazine-2,4 (1H,3H)-dione (8 g, 52.9 mmol) and DIPEA (13.66 g, 106 mmol) in toluene (80 mL) at 0° C. was added POCl₃ (19.7 mL, 212 mmol) drop wise. After the addition, the mixture was stirred at 120° C. for 16 h. LC-MS showed the reaction was complete. The reaction mixture was concentrated, then diluted with EA (200 mL). The organic phase was washed with saturated NaHCO₃ aqueous solution and brine, dried over anhydrous Na₂SO₄, filtered and concentrated. The residue was purified by column chromatography (PE:EA=3:1, V/V) to give the desired product (7.2 g, yield: 72%). LC/MS (ESI): m/z 188/190 [M+H]⁺.

### Step 6. 2-chloro-4-(1-methyl-1H-pyrazol-5-yl)imidazo[1,5-b]pyridazine

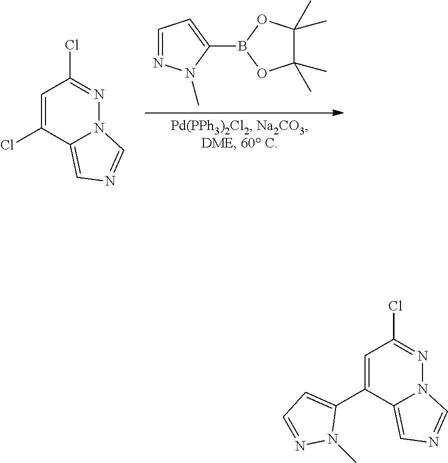 [SMILES: Clc1cc(Cl)c2cncn2n1 ; Cn1nccc1B1OC(C)(C)C(C)(C)O1 ; "Pd(PPh3)2Cl2, Na2CO3,DME, 60° C." ; Cn1nccc1-c1cc(Cl)nn2cncc12]

To a solution of 2,4-dichloroimidazo[1,5-b]pyridazine (1 g, 5.32 mmol) and 1-methyl-5-(4,4,5,5-tetramethyl-1,3,2-dioxaborolan-2-yl)-1H-pyrazole (1.44 g, 6.91 mmol) in DME (20 mL) were added bis(triphenylphosphine)palladium(II) chloride (0.83 g, 1.06 mmol) and Na₂CO₃ (2M in H₂O, 5.32 mL, 10.64 mmol). The reaction was charged with N₂ twice, then stirred at 60° C. overnight. LC-MS showed the reaction was complete. The mixture was diluted with EA (50 mL), then washed with water and brine, dried over anhydrous Na₂SO₄, filtered and concentrated. The residue was purified by column chromatography on silica gel (PE:EA=3:1, V/V) to give the desired product (500 mg, yield: 40%). LC/MS ESI (m/z): 234 [M+H]⁺.

### Step 7. (R)-3-methyl-4-(4-(1-methyl-1H-pyrazol-5-yl)imidazo[1,5-b]pyridazin-2-yl)morpholine

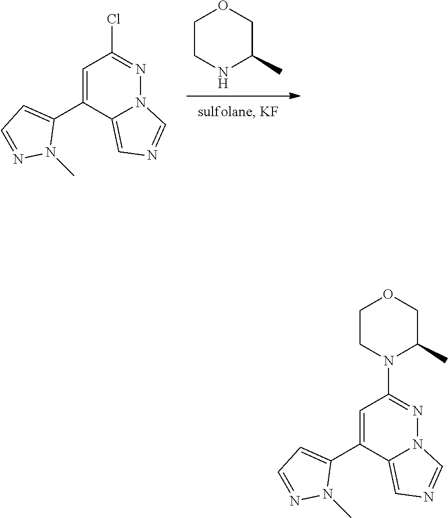

To a solution of 2-chloro-4-(1-methyl-1H-pyrazol-5-yl)imidazo[1,5-b]pyridazine (1 g, 4.28 mmol) in sulfolane (20 mL) was added (R)-3-methylmorpholine (1.30 g, 12.839 mmol) and KF (0.75 g, 12.839 mmol). The mixture was stirred at 180° C. for 8 h. LC-MS showed the reaction was complete. The mixture was diluted with EA (50 mL), then washed with water and brine, dried over anhydrous Na₂SO₄, filtered and concentrated. The residue was purified by column chromatography on silica gel (DCM:MeOH=20:1, V/V) to give the desired product (330 mg, yield: 26%). LC/MS ESI (m/z): 299 [M+H]⁺.

### Step 8. (3R)-4-[5,7-diiodo-4-(1-methyl-1H-pyrazol-5-yl)imidazo[1,5-b]pyridazin-2-yl]-3-methylmorpholine

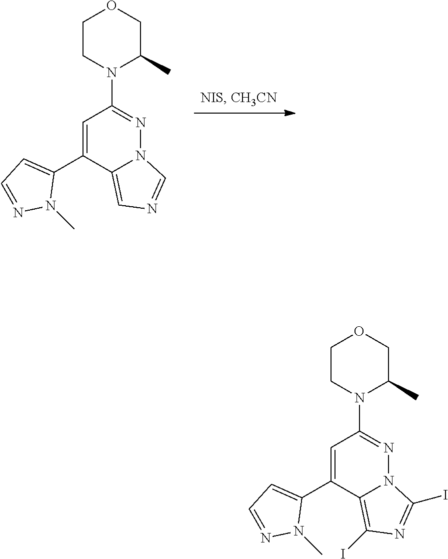

To a solution of (3R)-3-methyl-4-[4-(1-methyl-1H-pyrazol-5-yl)imidazo[1,5-b]pyridazin-2-yl]morpholine (230 mg, 0.77 mmol) in MeCN (15 mL) was added NIS (520.3 mg, 2.31 mmol). The mixture was stirred at room temperature for 2 h. LC-MS showed the reaction was complete. The mixture was diluted with EA (50 mL), then washed with water and brine, dried over anhydrous Na₂SO₄, filtered and concentrated. The residue was purified by column chromatography on silica gel (DCM:MeOH=20:1, V/V) to give the desired product (340 mg, yield: 80%). LC/MS ESI (m/z): 551 [M+H]⁺.

### Step 9. (3R)-4-[5-iodo-4-(1-methyl-1H-pyrazol-5-yl)-7-[1-(oxan-2-yl)-1H-pyrazol-5-yl]imidazo[1,5-b]pyridazin-2-yl]-3-methylmorpholine

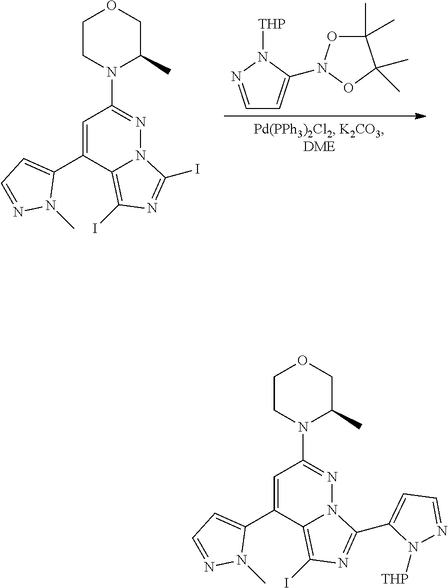

To a solution of (3R)-4-[5,7-diiodo-4-(1-methyl-1H-pyrazol-5-yl)imidazo[1,5-b] pyridazin-2-yl]-3-methylmorpholine (170 mg, 0.31 mmol) and 1-(oxan-2-yl)-5-(tetramethyl-1,3,2-dioxaborolan-2-yl)-1H-pyrazole (128.9 mg, 0.46 mmol) in co-solvent of DME (5 mL) and H₂O (1 mL) were added K₂CO₃ (42.7 mg, 0.31 mmol) and Pd(PPh₃)₂Cl₂ (43.4 mg, 0.06 mmol). The mixture was stirred at 80° C. overnight under nitrogen atmosphere. LC-MS showed the reaction was complete. The mixture was diluted with EA (50 mL), then washed with water and brine, dried over anhydrous Na₂SO₄, filtered and concentrated. The residue was purified by column chromatography on silica gel (PE:EA=1:1, V/V) to give the desired product (80 mg, yield: 45%). LC/MS ESI (m/z): 575 [M+H]⁺.

### Step 10. (3R)-3-methyl-4-[4-(1-methyl-1H-pyrazol-5-yl)-7-(1H-pyrazol-5-yl)imidazo [1,5-b]pyridazin-2-yl]morpholine

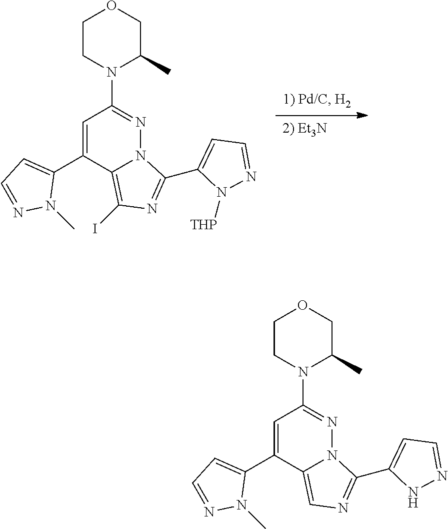

To a solution of (3R)-4-[5-iodo-4-(1-methyl-1H-pyrazol-5-yl)-7-[1-(oxan-2-yl)-1H-pyrazol-5-yl]imidazo[1,5-b]pyridazin-2-yl]-3-methylmorpholine (80 mg, 0.14 mmol) in MeOH (4 mL) was added Pd/C (10%, 20 mg). The mixture was stirred at room temperature for 12 h under H₂ atmosphere. A drop of Et₃N was added to the above solution, then the resulting mixture was continued to stir at room temperature for an additional 2 h under H₂ atmosphere. LC-MS showed the reaction was complete. The reaction mixture was filtered and concentrated. The residue was purified by Prep-HPLC (C18, 10-95%, MeOH in H₂O with 0.1% HCOOH) to give the desired product (12.4 mg, yield: 24%). LC/MS (ESI): m/z 365 [M+H]⁺. ¹H NMR (400 MHz, DMSO) δ 7.72 (s, 1H), 7.65 (d, J=1.9 Hz, 1H), 7.43 (s, 1H), 7.13 (d, J=1.9 Hz, 1H), 6.98 (s, 1H), 6.81 (d, J=1.9 Hz, 1H), 4.40 (d, J=6.4 Hz, 1H), 4.01 (d, J=8.2 Hz, 1H), 3.98 (s, 3H), 3.93 (d, J=12.7 Hz, 1H), 3.76 (dd, J=15.8, 7.0 Hz, 2H), 3.58 (dd, J=12.1, 9.3 Hz, 1H), 3.26 (s, 1H), 1.26 (d, J=6.7 Hz, 3H).

### Example 28

### Synthesis of (R)-3-methyl-4-(4-(1-methyl-1H-pyrazol-5-yl)-8-(1H-pyrazol-5-yl)pyrrolo[1,2-a]pyrimidin-2-yl)morpholine

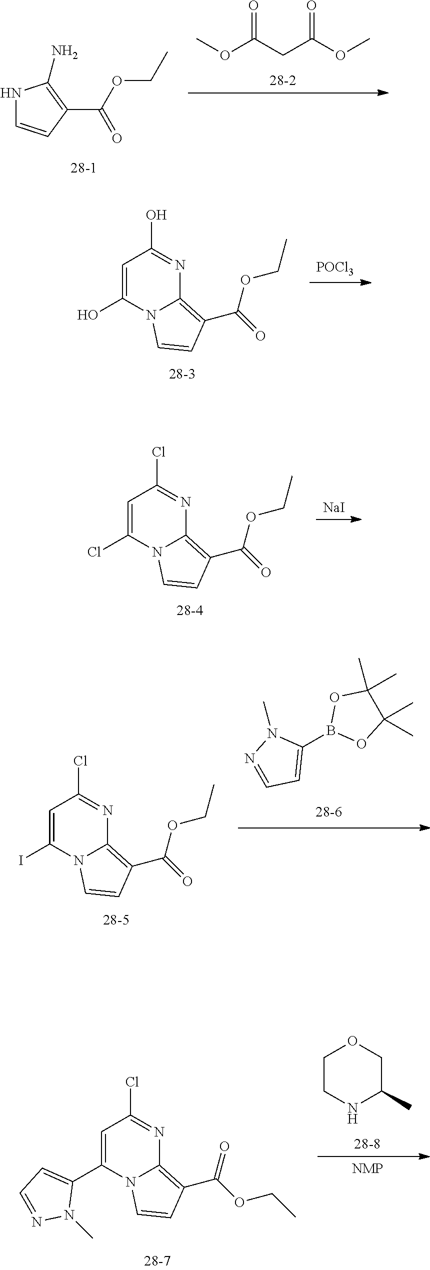

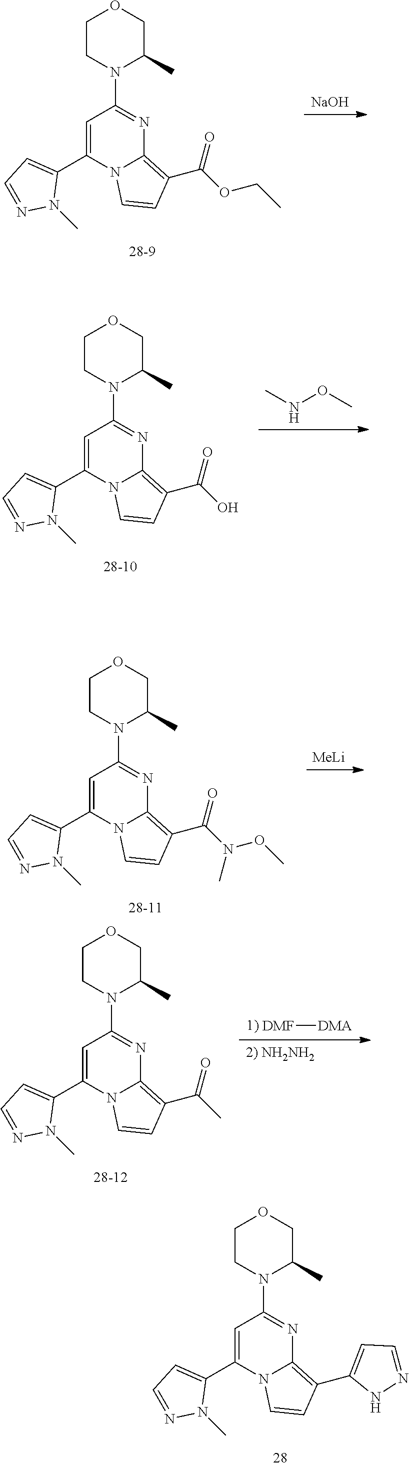

### Step 1. ethyl 2,4-dihydroxypyrrolo[1,2-a]pyrimidine-8-carboxylate

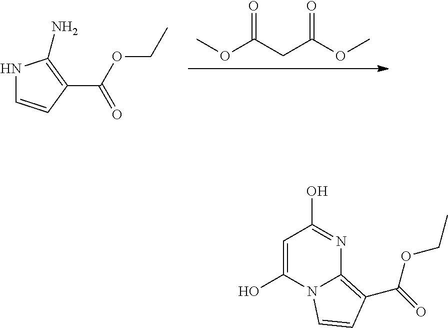

To a suspension of ethyl 2-amino-1H-pyrrole-3-carboxylate (2 g, 13.0 mmol) and Cs₂CO₃ (12.7 g, 38.9 mmol) in DMF (80 mL) was added 1,3-dimethyl propanedioate (3.7 mL, 32.4 mmol). The mixture was stirred at 120° C. for 6 h. LC-MS showed the reaction was complete. The reaction mixture was filtered, the filtrate was concentrated under reduced pressure to dryness. The residue was suspended in co-solvent of DCM (160 mL) and MeOH (40 mL), then stirred at room temperature for 0.5 h. The resulting mixture was filtered, the filtrate was concentrated under vacuo to give the crude product (2.8 g). LC/MS (ESI): m/z 223 [M+H]⁺.

### Step 2. ethyl 2,4-dichloropyrrolo[1,2-a]pyrimidine-8-carboxylate

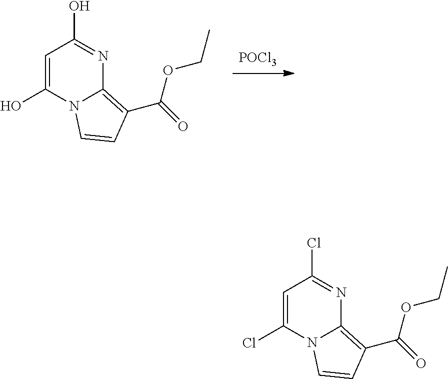

To a mixture of ethyl 2,4-dihydroxypyrrolo[1,2-a]pyrimidine-8-carboxylate (2.8 g, 12.6 mmol) in POCl₃ (40 mL) was stirred at 100° C. for 2 h. LC-MS showed the reaction was complete. The mixture was concentrated under reduced pressure to dryness, then diluted with DCM (80 mL). The resulting mixture was washed with saturated NaHCO₃ aqueous solution and brine, dried over anhydrous Na₂SO₄, filtered and concentrated. The residue was purified by column chromatography on silica gel (PE:EA=2:1, V/V) to afford the desired product (1.25 g, yield: 37%). LC/MS (ESI): m/z 259/261 [M+H]⁺.

### Step 3. ethyl 2-chloro-4-iodopyrrolo[1,2-a]pyrimidine-8-carboxylate

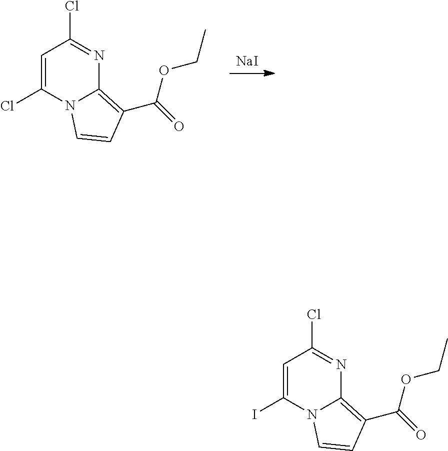

To a mixture of ethyl 2,4-dichloropyrrolo[1,2-a]pyrimidine-8-carboxylate (1.25 g, 4.82 mmol) in NMP (30 mL) was added NaI (3.62 g 24.1 mmol). The mixture was stirred at 120° C. for 4 h. LC-MS showed the reaction was complete. LC-MS showed the reaction was complete. The reaction mixture was diluted with DCM (20 mL), then washed with saturated Na₂S₂O₃ aqueous solution and brine, dried over anhydrous Na₂SO₄, filtered and concentrated. The residue was purified by column chromatography on silica gel (PE:EA=2:1, V/V) to afford the desired product (1.27 g, yield: 75%). LC/MS (ESI): m/z 351/353 [M+H]⁺.

### Step 4. ethyl 2-chloro-4-(1-methyl-1H-pyrazol-5-yl)pyrrolo[1,2-a]pyrimidine-8-carboxylate

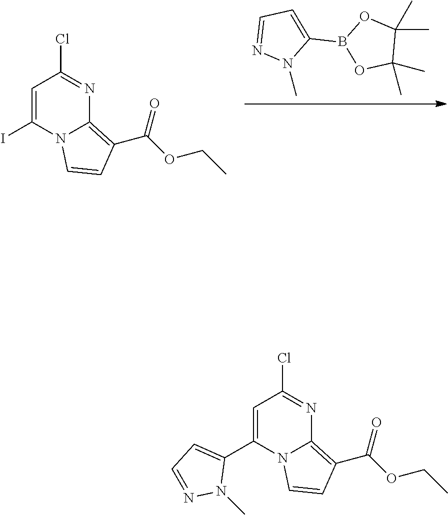 [SMILES: CCOC(=O)c1ccn2c(I)cc(Cl)nc12 ; Cn1nccc1B1OC(C)(C)C(C)(C)O1 ; CCOC(=O)c1ccn2c(-c3ccnn3C)cc(Cl)nc12]

To a solution of ethyl 2-chloro-4-iodopyrrolo[1,2-a]pyrimidine-8-carboxylate (600 mg, 1.71 mmol) and 1-methyl-5-(4,4,5,5-tetramethyl-1,3,2-dioxaborolan-2-yl)-1H-pyrazole (427 mg, 2.05 mmol) in DME (15 mL) were added Na₂CO₃ (2M in H₂O, 1.7 mL, 3.42 mmol) and Pd(PPh₃)₄ (198 mg, 0.17 mmol). The reaction was stirred at 40° C. overnight under nitrogen atmosphere. LC-MS showed the reaction was complete. The reaction mixture was diluted with DCM (30 mL), then washed with water and brine, dried over anhydrous Na₂SO₄, filtered and concentrated. The residue was purified by column chromatography on silica gel (PE:EA=2:1, V/V) to afford the desired product (400 mg, yield: 76%). LC/MS (ESI): m/z 305 [M+H]⁺.

### Step 5. ethyl (R)-4-(1-methyl-1H-pyrazol-5-yl)-2-(3-methylmorpholino)pyrrolo[1,2-a] pyrimidine-8-carboxylate

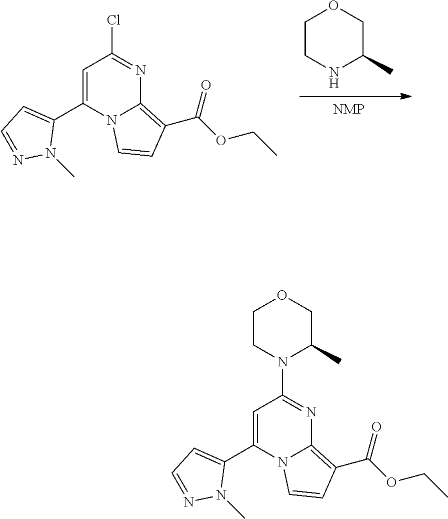

To a solution of ethyl 2-chloro-4-(1-methyl-1H-pyrazol-5-yl)pyrrolo[1,2-a]pyrimidine-8-carboxylate (400 mg, 1.31 mmol) in NMP (10 mL) was added (3R)-3-methylmorpholine (398 mg, 3.94 mmol). The reaction was stirred at 120° C. for 1 h under microwave irradiation. LC-MS showed the reaction was complete. The reaction mixture was diluted with DCM (20 mL), then washed with water and brine, dried over anhydrous Na₂SO₄, filtered and concentrated. The residue was purified by column chromatography on silica gel (PE:EA=2:1, V/V) to afford the desired product (300 mg, yield: 62%). LC/MS (ESI): m/z 370 [M+H]⁺.

### Step 6. (R)-4-(1-methyl-1H-pyrazol-5-yl)-2-(3-methylmorpholino)pyrrolo[1,2-a]pyrimidine-8-carboxylic acid

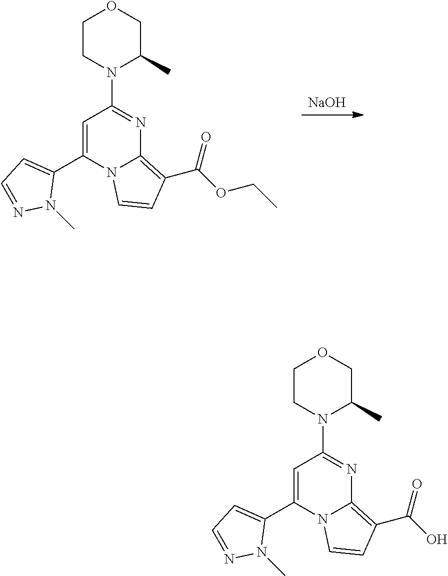 [SMILES: CCOC(=O)c1ccn2c(-c3ccnn3C)cc(N3CCOC[C@H]3C)nc12 ; "NaOH" ; C[C@@H]1COCCN1c1cc(-c2ccnn2C)n2ccc(C(=O)O)c2n1]

To a solution of ethyl (R)-4-(1-methyl-1H-pyrazol-5-yl)-2-(3-methylmorpholino) pyrrolo[1,2-a]pyrimidine-8-carboxylate (300 mg, 0.81 mmol) in co-solvent of MeOH (9 mL) and H₂O (3 mL) was added sodium hydroxide (162 mg, 4.06 mmol). The reaction was stirred at 70° C. overnight. LC-MS showed the reaction was complete. The reaction mixture was concentrated under vacuo to afford the crude product (250 mg). LC/MS (ESI): m/z 342 [M+H]⁺.

### Step 7. (R)—N-methoxy-N-methyl-4-(1-methyl-1H-pyrazol-5-yl)-2-(3-methyl morpholino)pyrrolo[1,2-a]pyrimidine-8-carboxamide

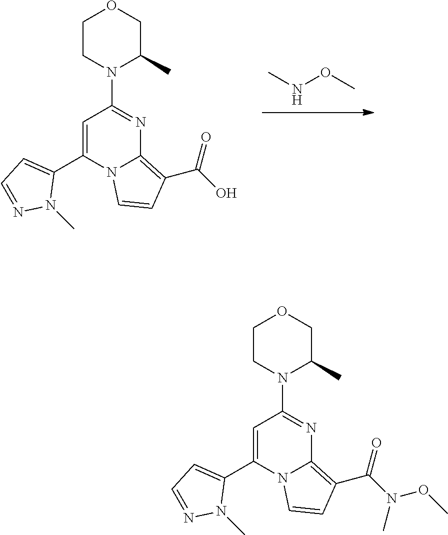

To a solution of (R)-4-(1-methyl-1H-pyrazol-5-yl)-2-(3-methylmorpholino)pyrrolo [1,2-a]pyrimidine-8-carboxylic acid (150 mg, 0.44 mmol) in DCM (20 mL) was added N,O-dimethylhydroxylamine (86 mg, 0.88 mmol), EDCI (126 mg, 0.66 mmol), HOBT (89 mg, 0.66 mmol) and TEA (0.31 mL, 2.20 mmol). The mixture was stirred at room temperature overnight. LC-MS showed the reaction was complete. The reaction mixture was diluted with DCM (20 mL), then washed with water and brine, dried over anhydrous Na₂SO₄, filtered and concentrated. The residue was purified by column chromatography on silica gel (PE:EA=1:1, V/V) to afford the desired product (85 mg, yield: 45%). LC/MS (ESI): m/z 385 [M+H]⁺.

### Step 8. (R)—N-methoxy-N-methyl-4-(1-methyl-1H-pyrazol-5-yl)-2-(3-methyl morpholino)pyrrolo[1,2-a]pyrimidine-8-carboxamide

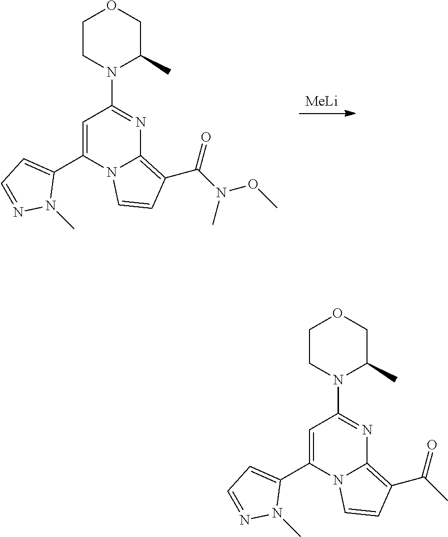 [SMILES: "MeLi" ; CON(C)C(=O)c1ccn2c(-c3ccnn3C)cc(N3CCOC[C@H]3C)nc12 ; CC(=O)c1ccn2c(-c3ccnn3C)cc(N3CCOC[C@H]3C)nc12]

To a solution of (R)—N-methoxy-N-methyl-4-(1-methyl-1H-pyrazol-5-yl)-2-(3-methylmorpholino)pyrrolo[1,2-a]pyrimidine-8-carboxamide (85 mg, 0.22 mmol) in THF (10 mL) at 0° C. was added methyllithium (1.3 M in THF, 1.7 mL, 2.21 mmol) drop wise. After the addition, the mixture was stirred at room temperature overnight. LC-MS showed the reaction was complete. The reaction mixture was quenched with saturated NH₄Cl aqueous solution, then extracted with EA twice (40 mL×2). The combined organic phase was washed with brine, dried over anhydrous Na₂SO₄, filtered and concentrated. The residue was purified by column chromatography on silica gel (PE:EA=2:1, V/V) to afford the desired product (30 mg, yield: 39%). LC/MS (ESI): m/z 340 [M+H]⁺.

### Step 9. (R)-3-methyl-4-(4-(1-methyl-1H-pyrazol-5-yl)-4-(1H-pyrazol-5-yl)pyrrolo [1,2-a]pyrimidin-2-yl)morpholine

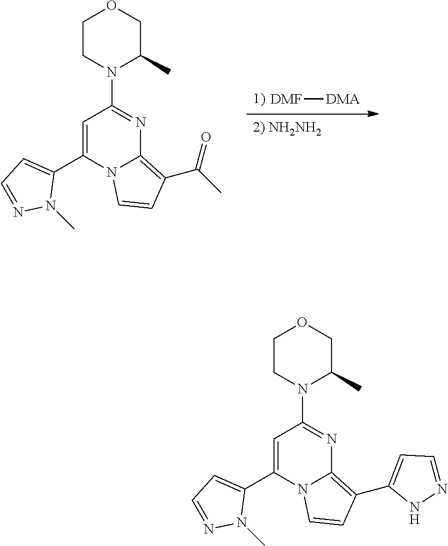

A mixture of (R)-1-(4-(1-methyl-1H-pyrazol-5-yl)-2-(3-methylmorpholino) pyrrolo [1,2-a]pyrimidin-8-yl)ethan-1-one (100 mg, 0.30 mmol) and N,N-Dimethylformamide dimethyl acetal (175 mg, 1.47 mmol) was stirred at 120° C. overnight. The reaction mixture was concentrated under vacuo. The residue was dissolved in EtOH (0.25 mL) and hydrazine hydrate (0.75 mL), then heated to 75° C. for 1 h. LC-MS showed the reaction was complete. The reaction mixture was diluted with DCM (20 mL), then washed with water and brine, dried over anhydrous Na₂SO₄, filtered and concentrated. The residue was purified by Prep-HPLC (C18, 10-95%, MeOH in H₂O with 0.1% HCOOH) to give the desired product (11 mg, yield: 10%). LC/MS (ESI): m/z 364 [M+H]⁺. ¹H NMR (400 MHz, DMSO) δ 12.56 (s, 1H), 7.70 (d, J=2.0 Hz, 1H), 7.51 (s, 1H), 7.09 (d, J=3.3 Hz, 1H), 6.81 (d, J=2.0 Hz, 1H), 6.79 (d, J=3.3 Hz, 1H), 6.76 (s, 1H), 6.73 (s, 1H), 4.49 (d, J=6.6 Hz, 1H), 4.13 (d, J=12.8 Hz, 1H), 3.99 (dd, J=11.3, 3.4 Hz, 1H), 3.85 (d, J=4.2 Hz, 3H), 3.77 (d, J=11.3 Hz, 1H), 3.67 (dd, J=11.4, 3.0 Hz, 1H), 3.52 (td, J=11.9, 2.9 Hz, 1H), 3.25-3.18 (m, 1H), 1.25 (d, J=6.7 Hz, 3H).

### Example 29

### Synthesis of (3R)-3-methyl-4-[7-(1-methyl-1H-pyrazol-5-yl)-3-(1H-pyrazol-5-yl)-[1,2]thiazolo[4,5-b]pyridin-5-yl]morpholine

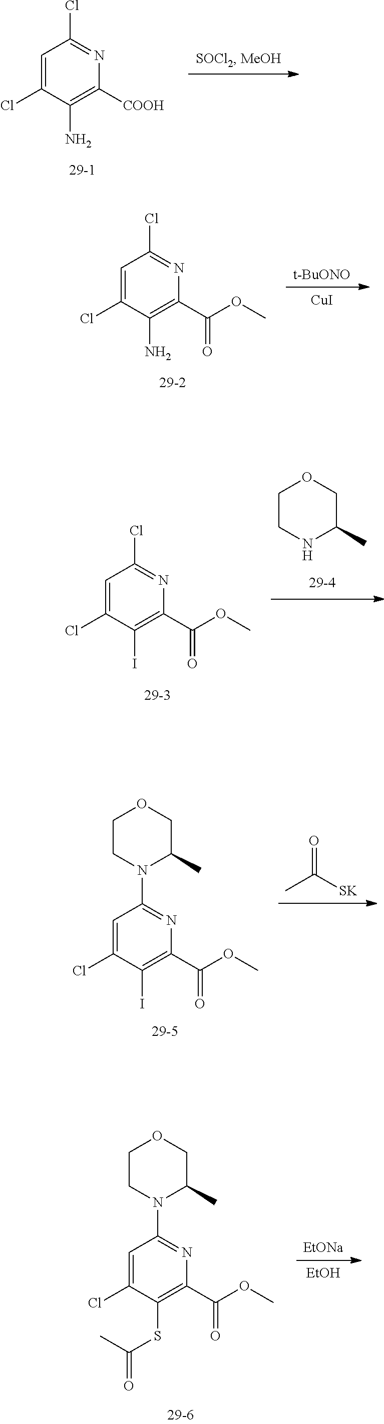

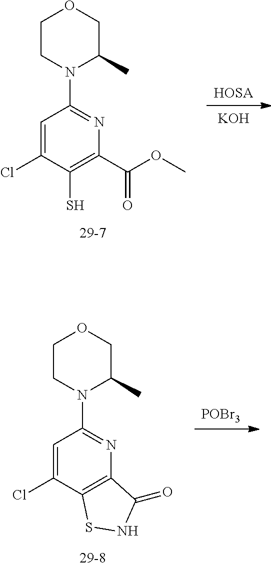 [SMILES: COC(=O)c1nc(N2CCOC[C@H]2C)cc(Cl)c1S ; "HOSA" ; "KOH" ; C[C@@H]1COCCN1c1cc(Cl)c2s[nH]c(=O)c2n1 ; "POBr3"]

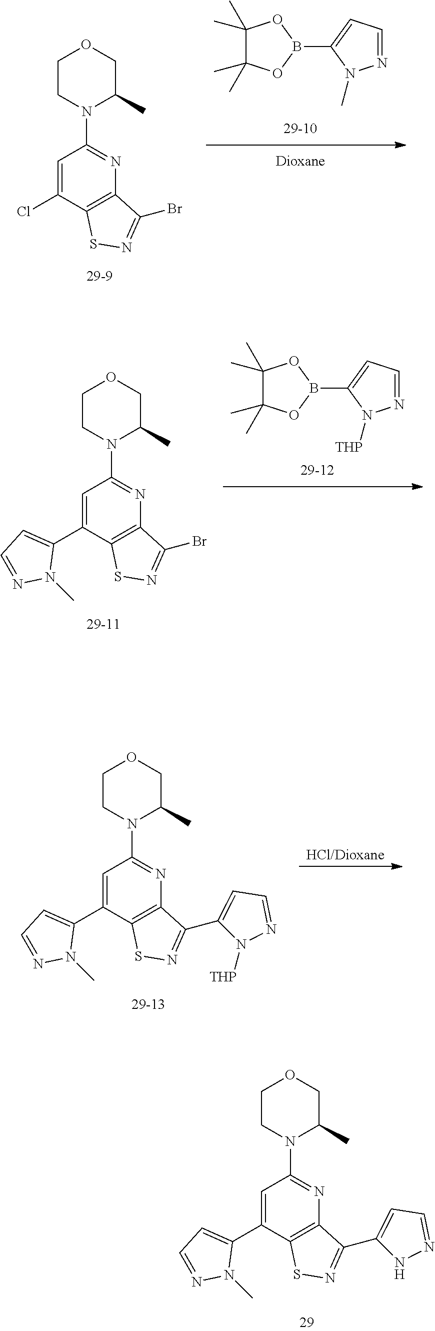

### Step 1. methyl 3-amino-4,6-dichloropyridine-2-carboxylate

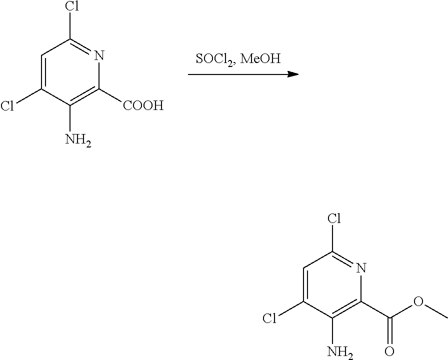

To a solution of 3-amino-4,6-dichloropyridine-2-carboxylic acid (10.0 g 48.30 mmol) in MeOH (150 mL) was added SOCl₂ (21.0 mL, 289.83 mmol) drop wise. After the addition, the mixture was stirred at 60° C. for 16 h. LCMS showed the reaction was completed. The reaction mixture was concentrated under reduced pressure, then diluted with DCM (100 mL). The organic phase was washed with saturated NaHCO₃ aqueous solution and brine, dried over anhydrous Na₂SO₄, filtered and concentrated. The residue was purified by column chromatography on silica gel (PE:EA=10:1) to give the desired product (10 g 94%). LC/MS (ESI): m/z 222 [M+H]⁺.

### Step 2. methyl 4,6-dichloro-3-iodopicolinate

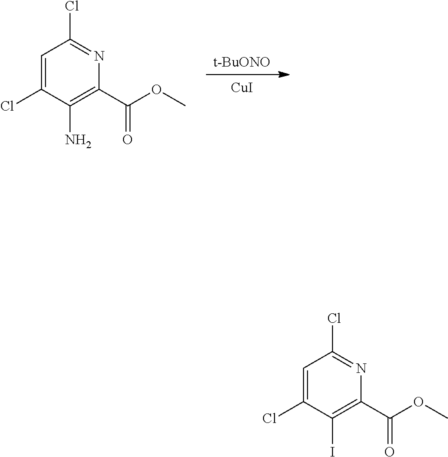

To a solution of methyl 3-amino-4,6-dichloropyridine-2-carboxylate (3.0 g, 13.57 mmol) and CuI (3.1 g, 16.29 mmol) in CH₃CN (130 mL) was added a solution of t-BuONO (2.1 g, 20.36 mmol) in CH₃CN (20 mL). After the addition, the mixture was stirred at 65° C. for 3 h. LC-MS showed the reaction was complete. The mixture was diluted with EA (50 mL), then washed with water and brine, dried over anhydrous Na₂SO₄, filtered and concentrated. The residue was purified by column chromatography on silica gel (PE:EA=10:1) to give the desired product (3 g, yield: 67%). LC/MS ESI (m/z): 332 [M+H]⁺.

### Step 3. methyl (R)-4-chloro-3-iodo-6-(3-methylmorpholino)picolinate

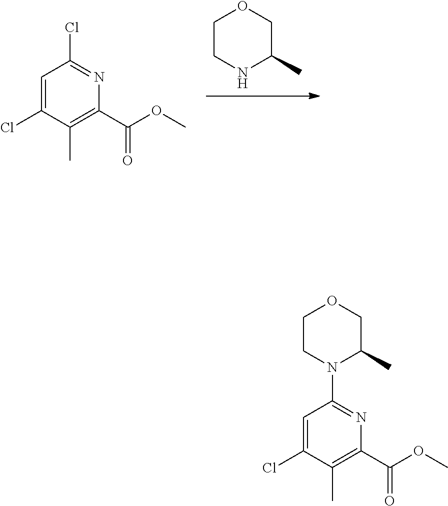

To a solution of methyl 4,6-dichloro-3-iodopyridine-2-carboxylate (1.0 g, 3.01 mmol) in NMP (15.0 mL) was added (3R)-3-methylmorpholine (0.9 g, 9.04 mmol). The mixture was stirred at 120° C. for 12 h. LC-MS showed the reaction was complete. The mixture was diluted with EA (50 mL), then washed with water and brine, dried over anhydrous Na₂SO₄, filtered and concentrated. The residue was purified by column chromatography on silica gel (PE:EA=10:1) to give the desired product (130 mg. yield: 11%). LC/MS ESI (m/z): 397 [M+H]⁺.

### Step 4. methyl (R)-3-(acetylthio)-4-chloro-6-(3-methylmorpholino)picolinate

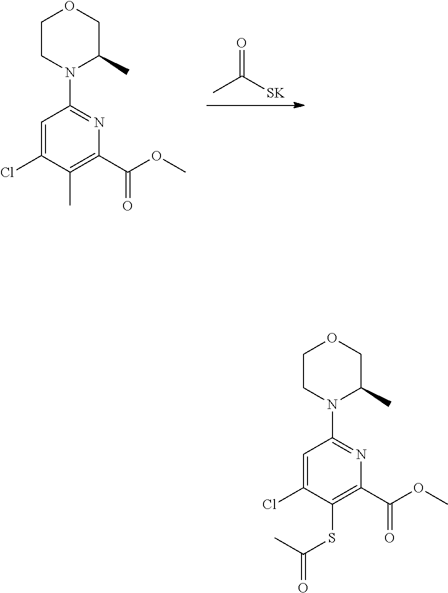

To a solution of 4-chloro-3-iodo-N-methoxy-N-methyl-6-[(3R)-3-methylmorpholin-4-yl]pyridine-2-carboxamide (160.0 mg, 0.38 mmol) and potassium thioacetate (128.8 mg, 1.13 mmol) in toluene (15.0 mL) were added CuI (38.4 mg, 0.20 mmol) and o-phenanthroline (72.7 mg, 0.40 mmol). The mixture was charged with N₂ twice, then stirred at 110° C. for 6 h. LC-MS showed the reaction was complete. The mixture was diluted with EA (50 mL), then washed with water and brine, dried over anhydrous Na₂SO₄, filtered and concentrated. The residue was purified by column chromatography on silica gel (PE:EA=3:1) to give the desired product (110 mg, yield: 79%). LC/MS ESI (m/z): 345 [M+H]⁺.

### Step 5. methyl (R)-4-chloro-3-mercapto-6-(3-methylmorpholino)picolinate

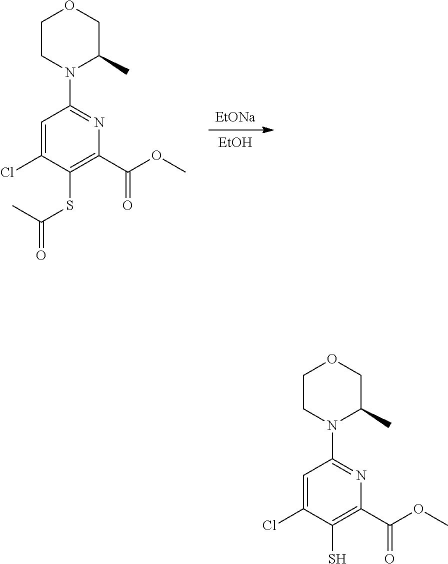 [SMILES: "EtONa" ; "EtOH" ; COC(=O)c1nc(N2CCOC[C@H]2C)cc(Cl)c1SC(C)=O ; COC(=O)c1nc(N2CCOC[C@H]2C)cc(Cl)c1S]

To a solution of methyl 3-(acetylsulfanyl)-4-chloro-6-[(3R)-3-methylmorpholin-4-yl] pyridine-2-carboxylate (70.0 mg, 0.20 mmol) in EtOH (4.0 mL) was added EtONa (20% in EtOH, 103.6 mg, 0.31 mmol). After the addition, the mixture was stirred at room temperature for 10 min. LC-MS showed the reaction was complete. The mixture was diluted with DCM (30 mL), then washed with water and brine, dried over anhydrous Na₂SO₄, filtered and concentrated. The residue was purified by column chromatography on silica gel (PE:EA=3:1) to give the desired product (50 mg, yield: 81%). LC/MS ESI (m/z): 303 [M+H]⁺.

### Step 6. (R)-7-chloro-5-(3-methylmorpholino)isothiazolo[4,5-b]pyridin-3 (2H)-one

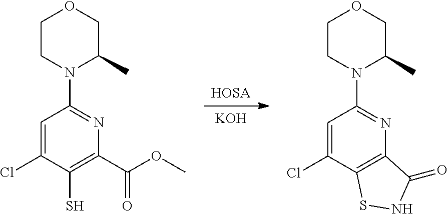 [SMILES: COC(=O)c1nc(N2CCOC[C@H]2C)cc(Cl)c1S ; "HOSA" ; "KOH" ; C[C@@H]1COCCN1c1cc(Cl)c2s[nH]c(=O)c2n1]

To a solvent of methyl 4-chloro-6-[(3R)-3-methylmorpholin-4-yl]-3-sulfanylpyridine-2-carboxylate (50 mg, 0.16 mmol) and KOH (18.5 mg, 0.34 mmol) in co-solvent of H₂O (2 mL) and THF (2 mL) was added a solution of HOSA (64.5 mg, 0.25 mmol) and KOH (27.8 mg, 0.51 mmol) in H₂O (1 mL) drop wise. After the addition, the mixture was stirred at room temperature for 12 h. LC-MS showed the reaction was complete. The mixture was diluted with DCM (30 mL), then washed with water and brine, dried over anhydrous Na₂SO₄, filtered and concentrated. The residue was purified by column chromatography on silica gel (PE:EA=1:1) to give the desired product (40 mg, yield: 84%). LC/MS ESI (m/z): 286 [M+H]⁺.

### Step 7. (R)-4-(3-bromo-7-chloroisothiazolo[4,5-b]pyridin-5-yl)-3-methylmorpholine

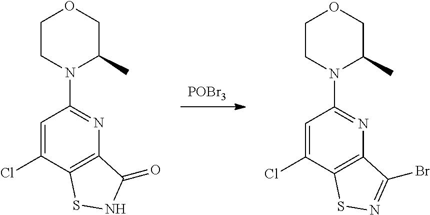 [SMILES: C[C@@H]1COCCN1c1cc(Cl)c2s[nH]c(=O)c2n1 ; "POBr3" ; C[C@@H]1COCCN1c1cc(Cl)c2snc(Br)c2n1]

A mixture of 7-chloro-5-[(3R)-3-methylmorpholin-4-yl]-2H,3H-[1,2]thiazolo[4,5-b] pyridin-3-one (40 mg, 0.14 mmol) and POBr₃ (1.2 g, 4.20 mmol) was stirred at 100° C. for 12 h. LC-MS showed the reaction was complete. After cooling to room temperature, the mixture was diluted with DCM (30 mL), then poured into ice-water. The organic layer was separated, then washed with brine, dried over anhydrous Na₂SO₄, filtered and concentrated. The residue was purified by column chromatography on silica gel (PE:EA=10:1) to give the desired product (20 mg, yield: 41%). LC/MS ESI (m/z): 348/350 [M+H]⁺.

### Step 8. (R)-4-(3-bromo-7-(1-methyl-1H-pyrazol-5-yl)isothiazolo[4,5-b]pyridin-5-yl)-3-methylmorpholine

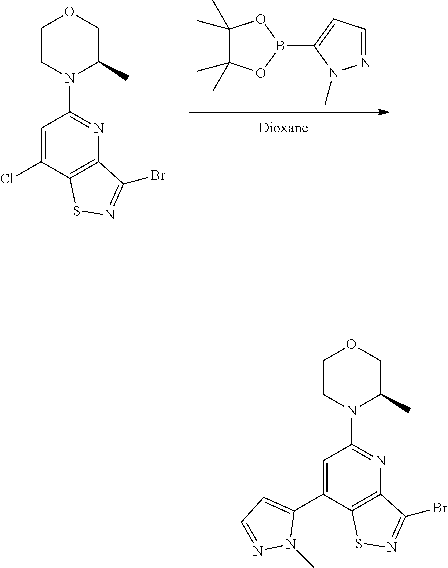 [SMILES: C[C@@H]1COCCN1c1cc(Cl)c2snc(Br)c2n1 ; Cn1nccc1B1OC(C)(C)C(C)(C)O1 ; C[C@@H]1COCCN1c1cc(-c2ccnn2C)c2snc(Br)c2n1]

To a solution of (3R)-4-{3-bromo-7-chloro-[1,2]thiazolo[4,5-b]pyridin-5-yl}-3-methylmorpholine (10.0 mg, 0.03 mmol) and 1-methyl-5-(tetramethyl-1,3,2-dioxaborolan-2-yl)-1H-pyrazole (9.0 mg, 0.04 mmol) in dioxane (1 mL) were added Pd(PPh₃)₄ (3.3 mg, 0.003 mmol) and Na₂CO₃ (2M in H₂O, 0.03 mL, 0.06 mmol). The mixture was charged with N₂ twice, then stirred at 100° C. for 12 h. LC-MS showed the reaction was complete. After cooling to room temperature, the mixture was diluted with DCM (30 mL), then washed with water and brine, dried over anhydrous Na₂SO₄, filtered and concentrated. The residue was purified by column chromatography on silica gel (PE:EA=10:1) to give the desired product (3 mg, yield: 27%). LC/MS ESI (m/z): 394/396 [M+H]⁺.

### Step 9. (3R)-3-methyl-4-(7-(1-methyl-1H-pyrazol-5-yl)-3-(1-(tetrahydro-2H-pyran-2-yl)-1H-pyrazol-5-yl)isothiazolo[4,5-b]pyridin-5-yl)morpholine

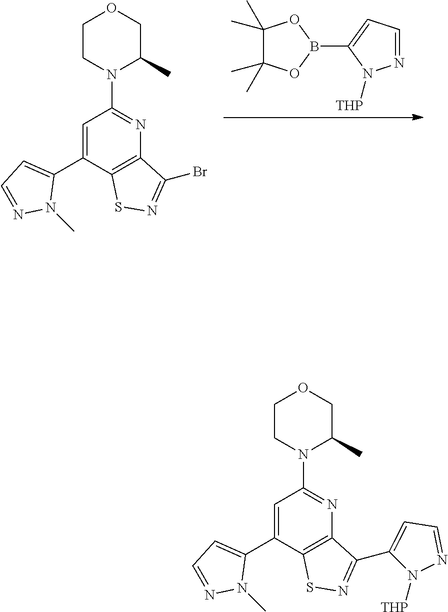

To a solution of (3R)-4-[3-bromo-7-(1-methyl-1H-pyrazol-5-yl)-[1,2]thiazolo[4,5-b] pyridin-5-yl]-3-methylmorpholine (3.0 mg, 0.01 mmol) and 1-(oxan-2-yl)-5-(tetramethyl-1,3,2-dioxaborolan-2-yl)-1H-pyrazole (4.2 mg, 0.02 mmol) in dioxane (1 mL) were added Pd(PPh₃)₄ (0.88 mg, 0.001 mmol) and K₂CO₃ (2M in H₂O, 0.01 mL, 0.02 mmol). The mixture was charged with N₂ twice, then stirred at 100° C. for 12 h. LC-MS showed the reaction was complete. After cooling to room temperature, the mixture was diluted with DCM (30 mL), then washed with water and brine, dried over anhydrous Na₂SO₄, filtered and concentrated. The residue was purified by column chromatography on silica gel (PE:EA=10:1) to give the desired product (1 mg, yield: 28%). LC/MS ESI (m/z): 466 [M+H]⁺.

### Step 10. (R)-3-methyl-4-(7-(1-methyl-1H-pyrazol-5-yl)-3-(1H-pyrazol-5-yl)isothiazolo[4,5-b]pyridin-5-yl)morpholine

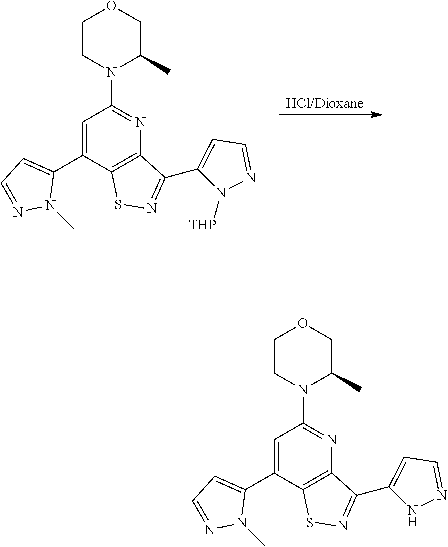

To a solution of (3R)-3-methyl-4-[7-(1-methyl-1H-pyrazol-5-yl)-3-[1-(oxan-2-yl)-1H-pyrazol-5-yl]-[1,2]thiazolo[4,5-b]pyridin-5-yl]morpholine (7.0 mg, 0.02 mmol) in DCM (1 mL) was added HCl solution (4M in dioxane, 1 mL). The resulting mixture was stirred at room temperature for 2 h. LC-MS showed the reaction was complete. The mixture was concentrated under reduced pressure, the residue was purified by prep-HPLC (C₁₈, 10-95%, MeOH in H₂O with 0.1% HCOOH) to give the desired product (3 mg, yield: 52%). LC/MS ESI (m/z): 382 [M+H]⁺. ¹H NMR (400 MHz, DMSO) δ 13.72 (s, 1H), 7.81 (d, J=77.1 Hz, 1H), 7.68 (d, J=2.0 Hz, 1H), 7.42 (d, J=1.9 Hz, 1H), 7.41 (s, 1H), 6.78 (d, J=1.9 Hz, 1H), 4.59 (d, J=4.6 Hz, 1H), 4.19 (d, J=13.4 Hz, 1H), 4.04 (dd, J=11.3, 3.0 Hz, 1H), 3.99 (s, 3H), 3.82 (d, J=11.3 Hz, 1H), 3.73 (dd, J=11.4, 2.9 Hz, 1H), 3.58 (td, J=11.9, 2.9 Hz, 1H), 3.31-3.24 (m, 1H), 1.26 (d, J=6.6 Hz, 3H).

### Example 30

### Synthesis of (R)-3-methyl-4-(4-(1-(methylsulfonyl)cyclopropyl)-7-(1H-pyrazol-5-yl) imidazo[1,5-b]pyridazin-2-yl)morpholine

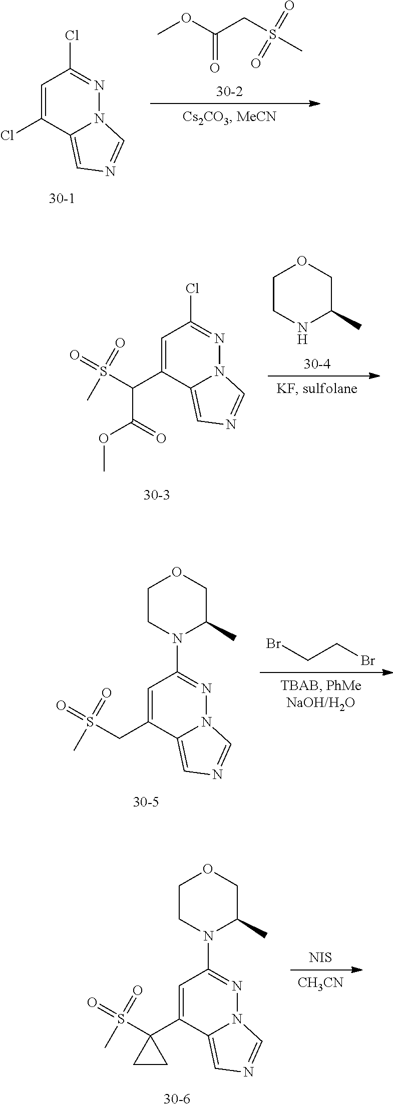

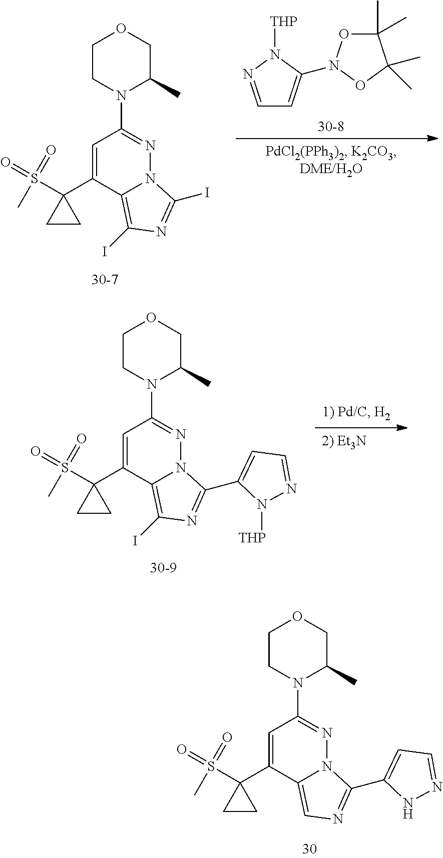

### Step 1. methyl 2-(2-chloroimidazo[1,5-b]pyridazin-4-yl)-2-(methylsulfonyl)acetate

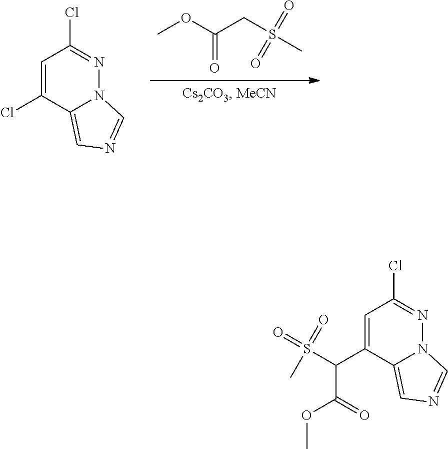

A mixture of 2,4-dichloroimidazo[1,5-b]pyridazine (500 mg, 2.65 mmol), methyl 2-methanesulfonylacetate (609 mg, 4.0 mmol) and Cs₂CO₃ (1.74 g, 5.34 mmol) in MeCN (10 mL) was stirred at 60° C. for 5 h. LC-MS showed the reaction was complete. The reaction mixture was diluted with DCM (20 mL), then washed with water and brine, dried over anhydrous Na₂SO₄, filtered and concentrated. The residue was purified by column chromatography on silica gel (EA) to afford the desired product (484 mg, yield: 60%). LC/MS(ESI): m/z 304 [M+H]⁺.

### Step 2. (R)-3-methyl-4-(4-((methylsulfonyl)methyl)imidazo[1,5-b]pyridazin-2-yl)morpholine

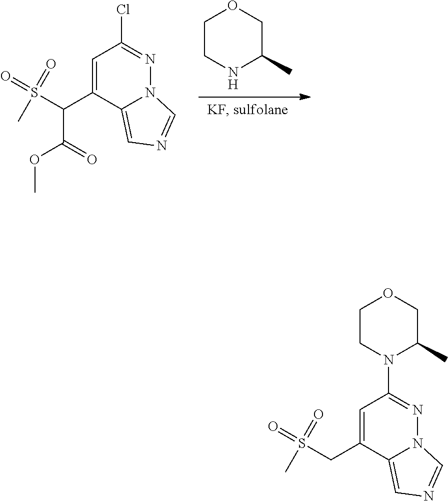

A mixture of methyl 2-{2-chloroimidazo[1,5-b]pyridazin-4-yl}-2-methanesulfonyl acetate (300 mg, 0.98 mmol), (3R)-3-methylmorpholine (400 mg, 3.95 mmol) and KF (170 mg, 58.0 mmol) in sulfolane (7 mL) was stirred at 180° C. for 7 h. LC-MS showed the reaction was complete. The reaction mixture was diluted with EA (40 mL), then washed with water and brine, dried over anhydrous Na₂SO₄, filtered and concentrated. The residue was purified by column chromatography on silica gel (DCM:MeOH=20:1, V/V) to afford the desired product (150 mg, yield: 49%). LC/MS(ESI): m/z 311 [M+H]⁺.

### Step 3. (R)-3-methyl-4-(4-(1-(methylsulfonyl)cyclopropyl)imidazo[1,5-b]pyridazin-2-yl)morpholine

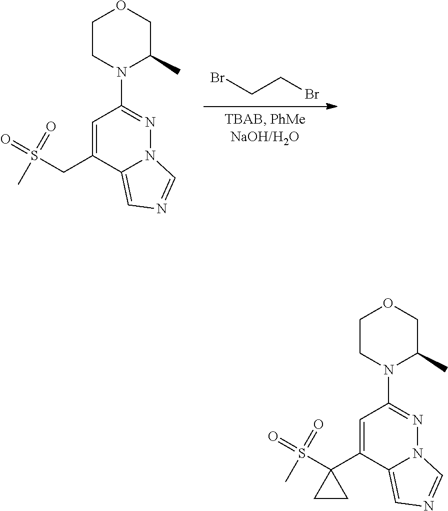

A mixture of (3R)-4-[4-(methanesulfonylmethyl)imidazo[1,5-b]pyridazin-2-yl]-3-methylmorpholine (274 mg, 0.88 mmol), 1,2-dibromoethane (657 mg, 3.49 mmol), TBAB (57 mg, 0.17 mmol) and NaOH (10 M in H₂O, 1.7 mL, 17.0 mmol) in toluene (10 mL) was stirred at 60° C. for 16 h. LC-MS showed the reaction was complete. The reaction mixture was diluted with EA (40 mL), then washed with water and brine, dried over anhydrous Na₂SO₄, filtered and concentrated. The residue was purified by column chromatography on silica gel (EA) to afford the desired product (106 mg, yield: 35%). LC/MS(ESI): m/z 337 [M+H]⁺.

### Step 4. (R)-4-(5,7-diiodo-4-(1-(methylsulfonyl)cyclopropyl)imidazo[1,5-b]pyridazin-2-yl)-3-methylmorpholine

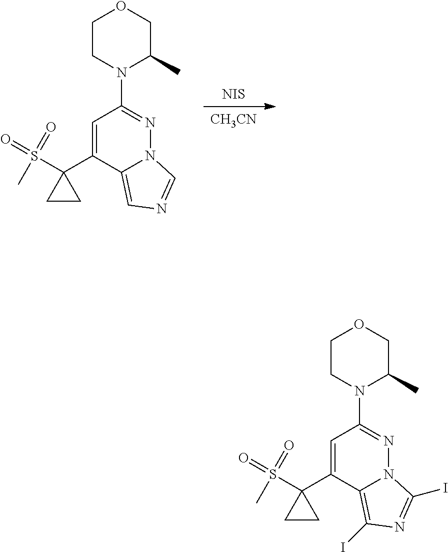

A mixture of (3R)-4-[4-(1-methanesulfonylcyclopropyl)imidazo[1,5-b]pyridazin-2-yl]-3-methylmorpholine (100 mg, 0.29 mmol) and NIS (267 mg, 1.18 mmol) in MeCN (5 mL) was stirred at room temperature for 16 h. LC-MS showed the reaction was complete. The reaction mixture was diluted with EA (40 mL), then washed with water and brine, dried over anhydrous Na₂SO₄, filtered and concentrated. The residue was purified by column chromatography on silica gel (PE:EA=1:1, V/V) to afford the desired product (171 mg, yield: 97%). LC/MS(ESI): m/z 589 [M+H]⁺.

### Step 5. (3R)-4-(5-iodo-4-(1-(methylsulfonyl)cyclopropyl)-7-(1-(tetrahydro-2H-pyran-2-yl)-1H-pyrazol-5-yl)imidazo[1,5-b]pyridazin-2-yl)-3-methylmorpholine

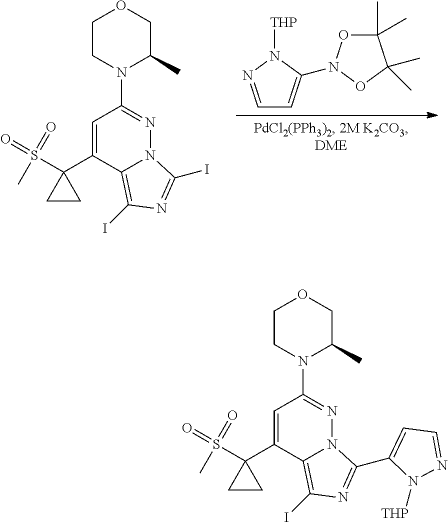

A mixture of (3R)-4-[5,7-diiodo-4-(1-methanesulfonylcyclopropyl)imidazo[1,5-b]pyridazin-2-yl]-3-methylmorpholine (160 mg, 0.27 mmol), 1-(oxan-2-yl)-5-(tetramethyl-1,3,2-dioxaborolan-2-yl)-1H-pyrazole (151 mg, 0.54 mmol), PdCl₂(PPh₃)₂ (38 mg, 0.05 mmol) and K₂CO₃ (2.0 M in H₂O, 0.4 mL, 0.80 mmol) in DME (5 mL) was stirred at 100° C. for 16 h under N₂ atmosphere. LC-MS showed the reaction was complete. The reaction mixture was diluted with EA (40 mL), then washed with water and brine, dried over anhydrous Na₂SO₄, filtered and concentrated. The residue was purified by column chromatography on silica gel (DCM:MeOH=20:1, V/V) to afford the desired product (60 mg, yield: 36%). LC/MS(ESI): m/z 613 [M+H]⁺.

### Step 6. (R)-3-methyl-4-(4-(1-(methylsulfonyl)cyclopropyl)-7-(1H-pyrazol-5-yl)imidazo[1,5-b]pyridazin-2-yl)morpholine

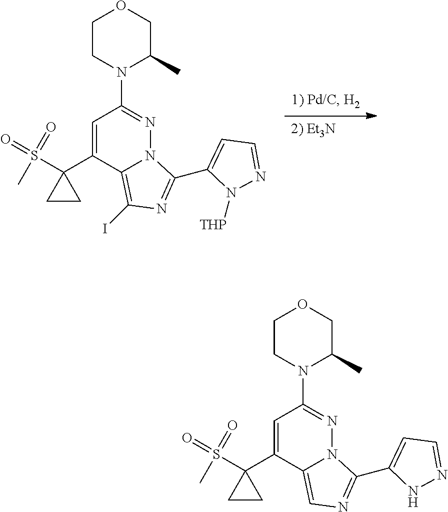

To a solution of (3R)-4-[5-iodo-4-(1-methanesulfonylcyclopropyl)-7-[1-(oxan-2-yl)-1H-pyrazol-5-yl]imidazo[1,5-b]pyridazin-2-yl]-3-methylmorpholine (70 mg, 0.11 mmol) in MeOH (5 mL) was added Pd/C (10%, 10 mg). The mixture was stirred at room temperature for 12 h under H₂ atmosphere. A drop of Et₃N was added to the above solution, then the resulting mixture was continued to stir at room temperature for an additional 2 h under H₂ atmosphere. LC-MS showed the reaction was complete. The reaction mixture was filtered and concentrated. The residue was purified by Prep-HPLC (C18, 10-95%, MeOH in H₂O with 0.1% HCOOH) to give the desired product (19 mg, yield: 41%). LC/MS (ESI): m/z 403 [M+H]⁺. ¹H NMR (400 MHz, DMSO) δ 13.27 (s, 1H), 7.70 (s, 1H), 7.59 (s, 1H), 7.11 (s, 1H), 7.09 (d, J=1.9 Hz, 1H), 4.34 (d, J=6.8 Hz, 1H), 4.00 (dd, J=11.4, 3.3 Hz, 1H), 3.89 (d, J=11.8 Hz, 1H), 3.78 (d, J=11.4 Hz, 1H), 3.71 (dd, J=11.4, 2.8 Hz, 1H), 3.56 (td, J=11.8, 2.9 Hz, 1H), 3.30-3.20 (m, 1H), 3.09 (s, 3H), 1.76 (dd, J=6.0, 4.3 Hz, 2H), 1.48 (t, J=5.2 Hz, 2H), 1.24 (d, J=6.7 Hz, 3H).

### Example 31

### Synthesis of (R)-3-methyl-4-(7-(3-methyl-1H-pyrazol-5-yl)-4-(1-(methylsulfonyl)cyclopropyl)imidazo[1,5-b]pyridazin-2-yl)morpholine

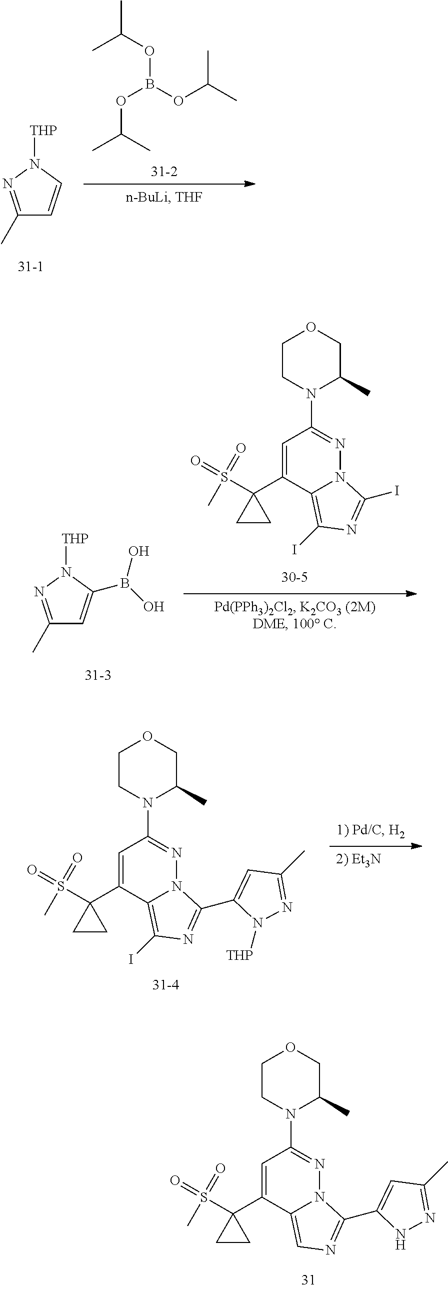

### Step 1. (3-methyl-1-(tetrahydro-2H-pyran-2-yl)-1H-pyrazol-5-yl)boronic acid

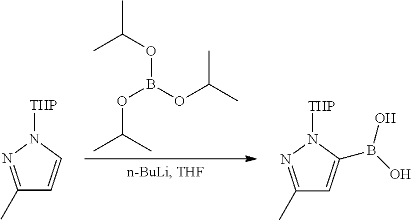

To a solution of 3-methyl-1-(oxan-2-yl)-1H-pyrazole (3 g, 18.1 mmol) in THF (40 mL) at −78° C. was added n-BuLi (2.5M in THF, 8 mL, 19.9 mmol) drop wise. The solution was stirred at −78° C. for 30 min, then tris(propan-2-yl) borate (5.01 mL, 21.7 mmol) was added slowly. The mixture was stirred at −78° C. for an additional 1 h, then HCl solution (2M, 18 mL, 36.1 mmol) was added. The resulting mixture was stirred at room temperature for 0.5 h. LC-MS showed the reaction was complete. The mixture was diluted with DCM (50 mL), then washed with water and brine, dried over anhydrous Na₂SO₄, filtered and concentrated. The residue was recrystallized from PE/EA (10:1, V/V) to give the desired product (1.3 g, yield: 34%). LC/MS ESI (m/z): 211 [M+H]⁺.

### Step 2. (3R)-4-(5-iodo-7-(3-methyl-1-(tetrahydro-2H-pyran-2-yl)-1H-pyrazol-5-yl)-4-(1-(methylsulfonyl)cyclopropyl)imidazo[1,5-b]pyridazin-2-yl)-3-methylmorpholine

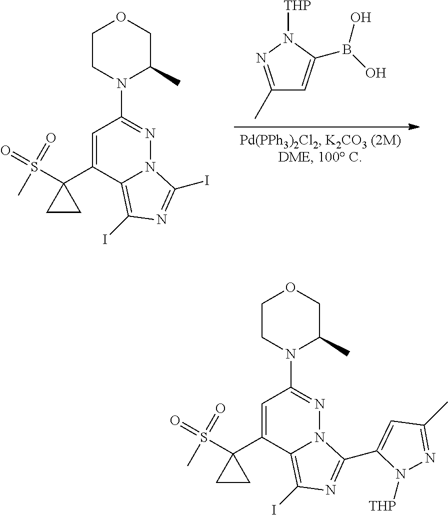

To a solution of (3R)-4-[5,7-diiodo-4-(1-methanesulfonylcyclopropyl)imidazo[1,5-b]pyridazin-2-yl]-3-methylmorpholine (150 mg, 0.26 mmol) and (3-methyl-1-(tetrahydro-2H-pyran-2-yl)-1H-pyrazol-5-yl)boronic acid (161 mg, 0.77 mmol) in DME (5 mL) were added K₂CO₃ (2M in H₂O, 0.38 mL, 0.765 mmol) and Pd(PPh₃)₂Cl₂ (18 mg, 0.026 mmol). The mixture was stirred at 100° C. for 16 h under N₂ atmosphere. LC-MS showed the reaction was complete. The mixture was diluted with DCM (50 mL), then washed with water and brine, dried over anhydrous Na₂SO₄, filtered and concentrated. The residue was purified by column chromatography on silica gel (PE:EA=1:1, V/V) to give the desired product (116 mg, yield: 73%). LC/MS ESI (m/z): 627 [M+H]⁺.

### Step 3. (R)-3-methyl-4-(7-(3-methyl-1H-pyrazol-5-yl)-4-(1-(methylsulfonyl)cyclopropyl)imidazo[1,5-b]pyridazin-2-yl)morpholine

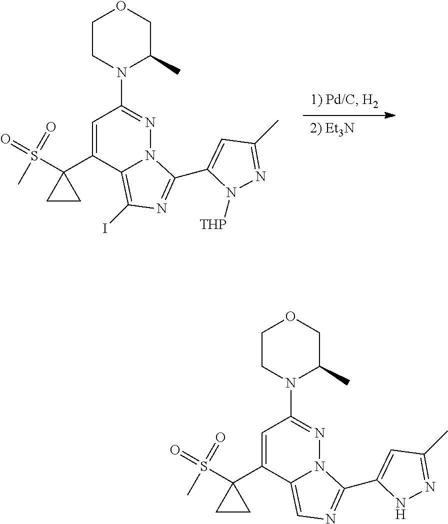

To a solution of (3R)-4-(5-iodo-7-(3-methyl-1-(tetrahydro-2H-pyran-2-yl)-1H-pyrazol-5-yl)-4-(1-(methylsulfonyl)cyclopropyl)imidazo[1,5-b]pyridazin-2-yl)-3-methylmorpholine (116 mg, 0.185 mmol) in MeOH (6 mL) was added Pd/C (10%, 20 mg). The mixture was stirred at room temperature for 12 h under H₂ atmosphere. A drop of Et₃N was added to the above solution, then the resulting mixture was continued to stir at room temperature for an additional 2 h under H₂ atmosphere. LC-MS showed the reaction was complete. The reaction mixture was filtered and concentrated. The residue was purified by Prep-HPLC (C18, 10-95%, MeOH in H₂O with 0.1% HCOOH) to give the desired product (12.2 mg, yield: 16%). LC/MS (ESI): m/z 417 [M+H]⁺. ¹H NMR (400 MHz, DMSO) δ 12.91 (s, 1H), 7.56 (s, 1H), 7.08 (s, 1H), 6.83 (s, 1H), 4.33 (dd, J=13.0, 6.6 Hz, 1H), 4.00 (dd, J=11.4, 3.3 Hz, 1H), 3.88 (dd, J=13.4, 1.1 Hz, 1H), 3.74 (dt, J=11.5, 7.1 Hz, 2H), 3.56 (td, J=11.7, 2.7 Hz, 1H), 3.25-3.21 (m, 1H), 3.08 (s, 3H), 2.28 (s, 3H), 1.79-1.70 (m, 2H), 1.52-1.43 (m, 2H), 1.24 (d, J=6.7 Hz, 3H).

### Example 32

### Synthesis of (1R,5S)-3-(4-(1-(methylsulfonyl)cyclopropyl)-7-(1H-pyrazol-5-yl)imidazo[1,5-b]pyridazin-2-yl)-8-oxa-3-azabicyclo[3.2.1]octane

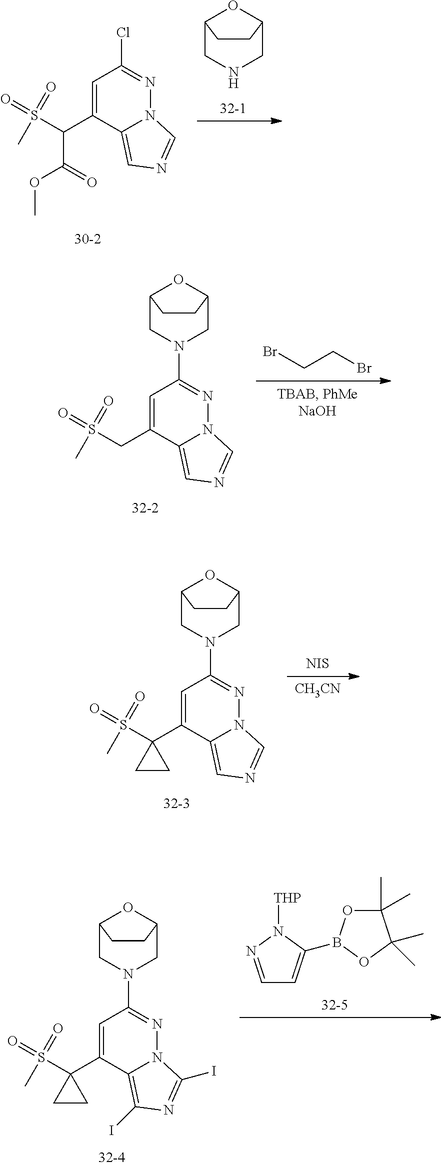

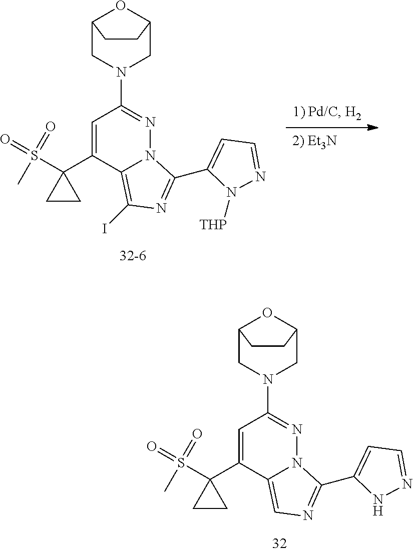

### Step 1. (1R,5S)-3-(4-((methylsulfonyl)methyl)imidazo[1,5-b]pyridazin-2-yl)-8 oxa-3-azabicyclo[3.2.1]octane

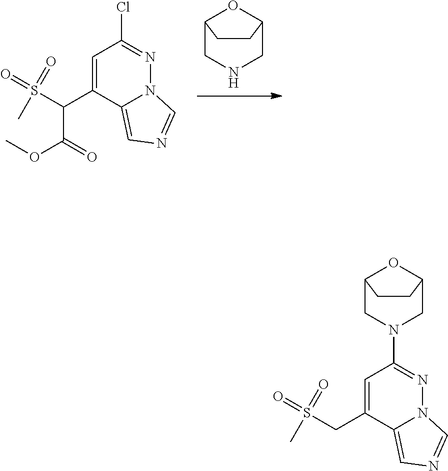 [SMILES: COC(=O)C(c1cc(Cl)nn2cncc12)S(C)(=O)=O ; C1CC2CNCC1O2 ; CS(=O)(=O)Cc1cc(N2CC3CCC(C2)O3)nn2cncc12]

To a suspension of methyl 2-{2-chloroimidazo[1,5-b]pyridazin-4-yl}-2-methane sulfonylacetate (600 mg, 1.98 mmol) and KF (573 mg, 9.88 mmol) in sulfolane (10 mL) was added 8-oxa-3-azabicyclo[3.2.1]octane (671 mg, 5.93 mmol). The mixture was stirred at 180° C. for 5 h. LC-MS showed the reaction was complete. The mixture was diluted with DCM (50 mL), then washed with water and brine, dried over anhydrous Na₂SO₄, filtered and concentrated. The residue was purified by column chromatography on silica gel (PE:EA=1:1, V/V) to give the desired product (181 mg, yield: 28%). LC/MS ESI (m/z): 323 [M+H]⁺.

### Step 2. (1R,5S)-3-(4-(1-(methylsulfonyl)cyclopropyl)imidazo[1,5-b]pyridazin-2-yl)-8-oxa-3-azabicyclo[3.2.1]octane

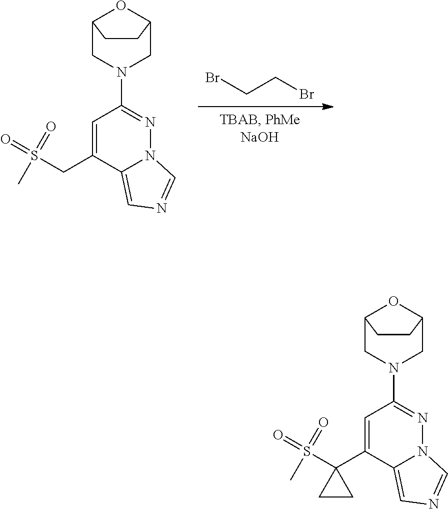

To a solution of (1R,5S)-3-(4-((methylsulfonyl)methyl)imidazo[1,5-b]pyridazin-2-yl)-8-oxa-3-azabicyclo[3.2.1]octane (181 mg, 0.561 mmol), 1,2-dibromoethane (1.05 g, 5.61 mmol) and TBAB (36 mg, 0.112 mmol) in toluene (8 mL) was added NaOH solution (10M in H₂O, 1.1 mL, 11.2 mmol). The mixture was stirred at 60° C. for 3 h. LC-MS showed the reaction was complete. The reaction mixture was poured into H₂O (40 mL) and extracted with DCM (30 mL×3). The combined organic phase was washed with brine, dried over anhydrous Na₂SO₄, filtered and concentrated. The residue was purified by column chromatography on silica gel (PE:EA=1:1, V/V) to give the desired product (153 mg, yield: 78%). LC/MS ESI (m/z): 349 [M+H]⁺.

### Step 3. (1R,5S)-3-(5,7-diiodo-4-(1-(methylsulfonyl)cyclopropyl)imidazo[1,5-b]pyridazin-2-yl)-8-oxa-3-azabicyclo[3.2.1]octane

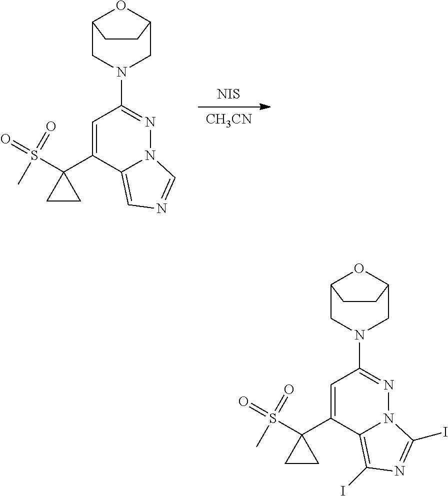

To a solution of (1R,5S)-3-(4-(1-(methylsulfonyl)cyclopropyl)imidazo[1,5-b]pyridazin-2-yl)-8-oxa-3-azabicyclo[3.2.1]octane (153 mg, 0.44 mmol) in MeCN (8 mL) was added NIS (395 mg, 1.76 mmol) portion wise. The mixture was stirred at 80° C. for 4 h. LC-MS showed the reaction was complete. The reaction mixture was quenched with saturated Na₂S₂O₃ aqueous solution and extracted with DCM (30 mL×3). The combined organic phase was washed with brine, dried over anhydrous Na₂SO₄, filtered and concentrated. The residue was purified by column chromatography on silica gel (PE:EA=1:1, V/V) to give the desired product (260 mg, yield: 98%). LC/MS ESI (m/z): 601 [M+H]⁺.

### Step 4. 3-[5-iodo-4-(1-methanesulfonylcyclopropyl)-7-[1-(oxan-2-yl)-1H-pyrazol-5-yl]imidazo[1,5-b]pyridazin-2-yl]-8-oxa-3-azabicyclo[3.2.1]octane

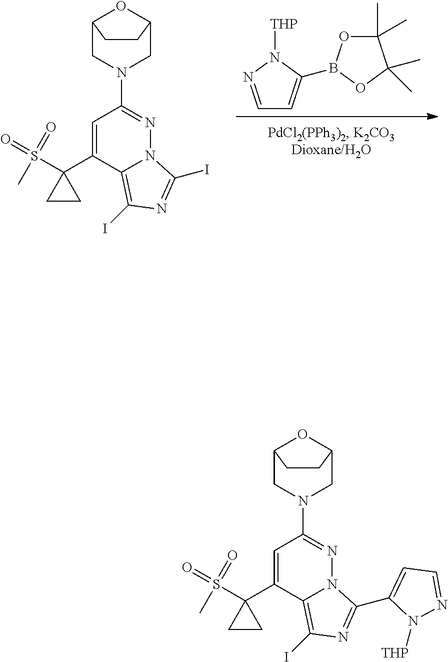

To a solution of 3-[5,7-diiodo-4-(1-methanesulfonylcyclopropyl)imidazo[1,5-b] pyridazin-2-yl]-8-oxa-3-azabicyclo[3.2.1]octane (145 mg, 0.24 mmol) and 1-(oxan-2-yl)-5-(tetramethyl-1,3,2-dioxaborolan-2-yl)-1H-pyrazole (87.4 mg, 0.31 mmol) in co-solvent of Dioxane (7 mL) and H₂O (0.7 mL) were added PdCl₂(PPh₃)₂ (17.0 mg, 0.02 mmol) and K₂CO₃ (100.0 mg, 0.73 mmol). The mixture was stirred at 100° C. overnight under N₂ atmosphere. LC-MS showed the reaction was complete. The reaction mixture was poured into H₂O (30 mL) and extracted with DCM twice (30 mL×2). The combined organic phase was washed with brine, dried over anhydrous Na₂SO₄, filtered and concentrated. The residue was purified by column chromatography on silica gel (DCM:MeOH=20:1, V/V) to give the desired product (60 mg, yield: 40%). LC/MS ESI (m/z): 625 [M+H]⁺.

### Step 5. 3-[4-(1-methanesulfonylcyclopropyl)-7-(1H-pyrazol-5-yl)imidazo[1,5-b]pyridazin-2-yl]-8-oxa-3-azabicyclo[3.2.1]octane

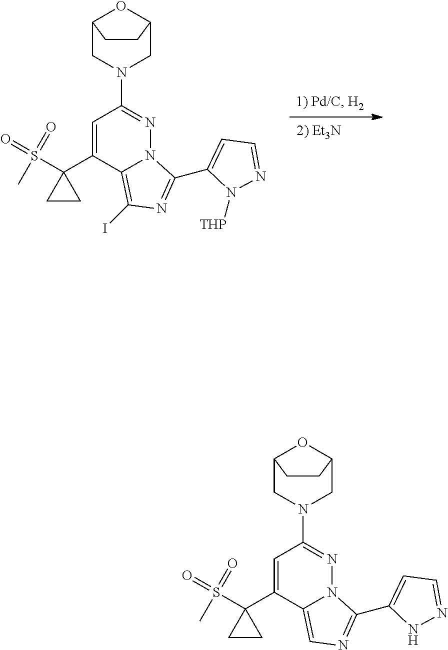

To a solution of (1R,5S)-3-(5-iodo-4-(1-(methylsulfonyl)cyclopropyl)-7-(1-(tetrahydro-2H-pyran-2-yl)-1H-pyrazol-5-yl)imidazo[1,5-b]pyridazin-2-yl)-8-oxa-3-azabicyclo[3.2.1]octane (60 mg, 0.1 mmol) in MeOH (6 mL) was added Pd/C (10%, 10 mg). The mixture was stirred at room temperature for 12 h under H₂ atmosphere. A drop of Et₃N was added to the above solution, then the resulting mixture was continued to stir at room temperature for an additional 2 h under H₂ atmosphere. LC-MS showed the reaction was complete. The reaction mixture was filtered and concentrated. The residue was purified by Prep-HPLC (C18, 10-95%, MeOH in H₂O with 0.1% HCOOH) to give the desired product (9.1 mg, yield: 23%). LC/MS (ESI): m/z 415 [M+H]⁺. ¹H NMR (400 MHz, DMSO) δ 13.31 (s, 1H), 7.69 (s, 1H), 7.57 (s, 1H), 7.09 (d, J=1.8 Hz, 1H), 7.05 (s, 1H), 4.50 (s, 2H), 3.87 (d, J=12.3 Hz, 2H), 3.17-3.13 (m, 2H), 3.09 (s, 3H), 1.91-1.81 (m, 4H), 1.75 (q, J=5.0 Hz, 2H), 1.48 (q, J=5.4 Hz, 2H).

### Example 33

### Synthesis of (3R)-4-[4-(dimethyl-1H-1,2,3-triazol-5-yl)-7-(1H-pyrazol-5-yl)imidazo[1,5-b]pyridazin-2-yl]-3-methylmorpholine

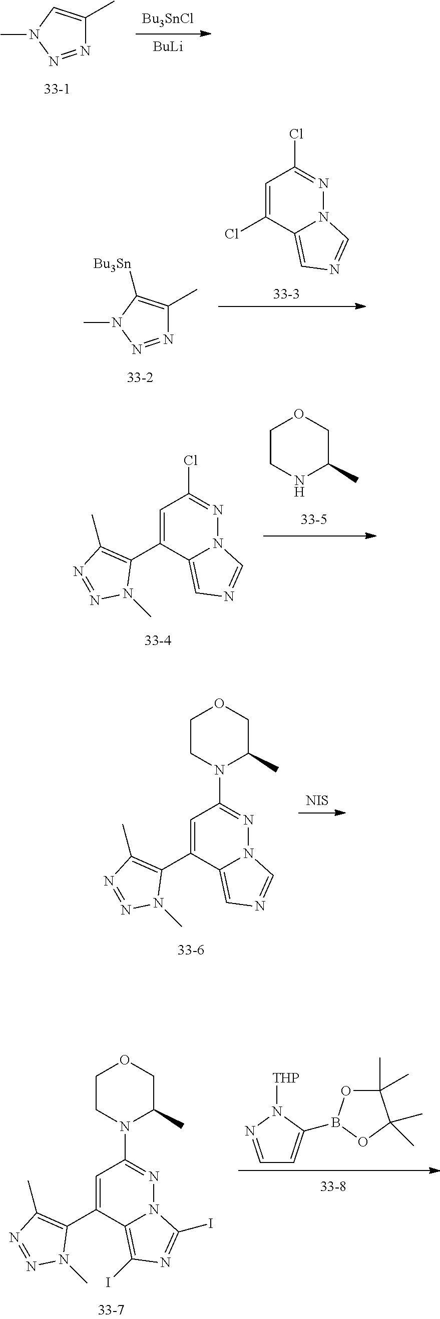

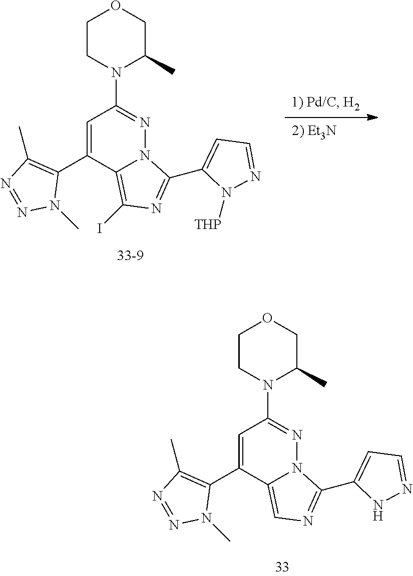

### Step 1. 1,4-dimethyl-5-(tributylstannyl)-1H-1,2,3-triazole

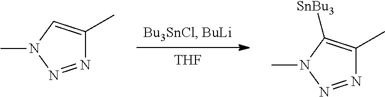

To a solution of n-BuLi (2.5M in THF, 27.7 mL, 69.19 mmol) in THF (300 mL) at −78° C. was added a solution of 1,4-dimethyl-1H-1,2,3-triazole (5.60 g, 57.66 mmol) in THF (50 mL) drop wise under nitrogen atmosphere. The mixture was stirred at −78° C. for 1 h, then tributyltin chloride (17.2 mL, 63.43 mmol) was added drop wise. The resulting mixture was stirred at −78° C. for 30 min, then gradually warmed to room temperature for an additional 1 h. LC-MS showed the reaction was complete. The reaction mixture was quenched with saturated NH₄Cl aqueous solution (200 mL), then extracted with EA twice (100 mL×2). The combined organic phase was washed with brine, dried over anhydrous Na₂SO₄, filtered and concentrated. The residue was purified by flash column chromatography (PE:EA=10:1) to give the desired product (17.0 g, yield: 76%). LC/MS (ESI): m/z 388 [M+H]⁺.

### Step 2. 5-{2-chloroimidazo[1,5-b]pyridazin-4-yl}-1,4-dimethyl-1H-1,2,3-triazole

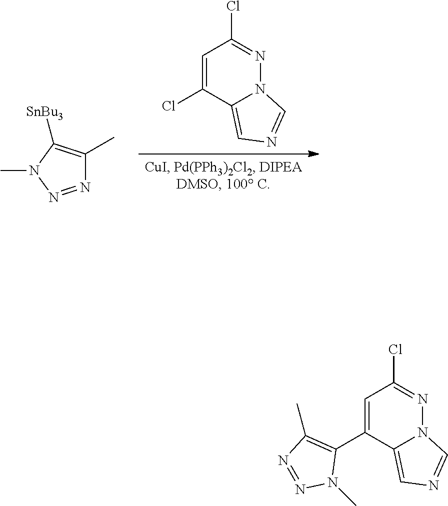

To a solution of 2,4-dichloroimidazo[1,5-b]pyridazine (1 g, 5.32 mmol) and 1,4-dimethyl-5-(tributylstannyl)-1H-1,2,3-triazole (3.1 g, 7.98 mmol) in DMSO (40 mL) were added CuI (0.1 g, 0.53 mmol), PdCl₂(PPh₃)₂ (0.37 g, 0.53 mmol) and DIPEA (2.2 mL, 13.30 mmol). The mixture was stirred at 100° C. overnight under nitrogen atmosphere. LC-MS showed the reaction was complete. The reaction mixture was diluted with EA (50 mL), then washed with water and brine, dried over anhydrous Na₂SO₄, filtered and concentrated. The residue was purified by flash column chromatography (PE:EA=3:1) to give the desired product (320 mg, yield: 24%). LC/MS (ESI): m/z 249 [M+H]⁺.

### Step 3. (3R)-4-[4-(dimethyl-1H-1,2,3-triazol-5-yl)imidazo[1,5-b]pyridazin-2-yl]-3-methylmorpholine

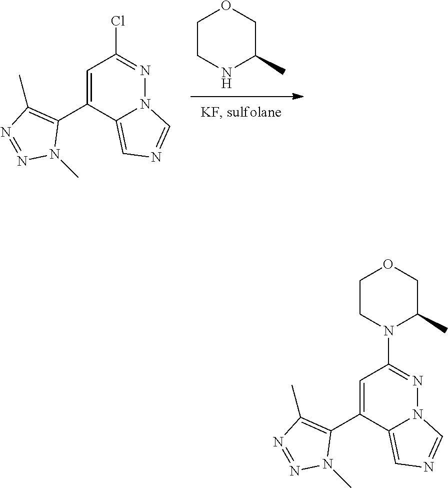

To a solution of 5-{2-chloroimidazo[1,5-b]pyridazin-4-yl}-1,4-dimethyl-1H-1,2,3-triazole (320 mg, 1.29 mmol) and (3R)-3-methylmorpholine (520.6 mg, 5.15 mmol) in sulfolane (3 mL) was added KF (224.2 mg, 3.86 mmol). The mixture was stirred at 180° C. for 8 h in a sealed tube. LC-MS showed the reaction was complete. The reaction mixture was diluted with EA (50 mL), then washed with water and brine, dried over anhydrous Na₂SO₄, filtered and concentrated. The residue was purified by Prep-HPLC (C18, 10-95%, MeCN in H₂O with 0.1% HCOOH) to give the desired product (134 mg, yield: 33%). LC/MS (ESI): m/z 314 [M+H]⁺.

### Step 4. (3R)-4-[4-(dimethyl-1H-1,2,3-triazol-5-yl)-5,7-diiodoimidazo[1,5-b]pyridazin-2-yl]-3-methylmorpholine

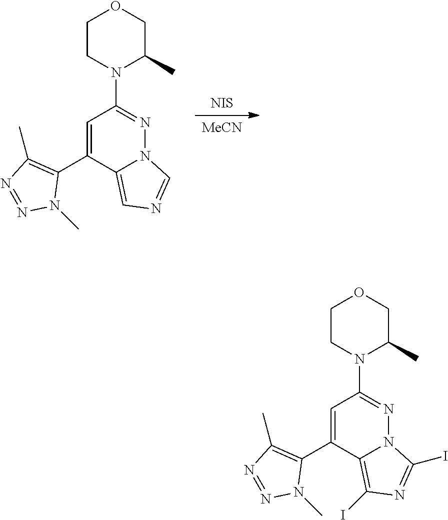

To a solution of (3R)-4-[4-(dimethyl-1H-1,2,3-triazol-5-yl)imidazo[1,5-b]pyridazin-2-yl]-3-methylmorpholine (134 mg, 0.43 mmol) in CH₃CN (10 mL) was added NIS (384.8 mg, 1.71 mmol). The resulting mixture was stirred at room temperature for 1 h. LC-MS showed the reaction was complete. The mixture was quenched with saturated Na₂S₂O₃ aqueous solution, then extracted with EA (50 mL). The organic layer was washed with brine, dried over Na₂SO₄, filtered and concentrated. The residue was purified by flash column chromatography (DCM:MeOH=20:1) to give the desired product (209 mg, yield: 86%). LC/MS (ESI): m/z 566 [M+H]⁺.

### Step 5. (3R)-4-[4-(dimethyl-1H-1,2,3-triazol-5-yl)-5-iodo-7-[1-(oxan-2-yl)-1H-pyrazol-5-yl]imidazo[1,5-b]pyridazin-2-yl]-3-methylmorpholine

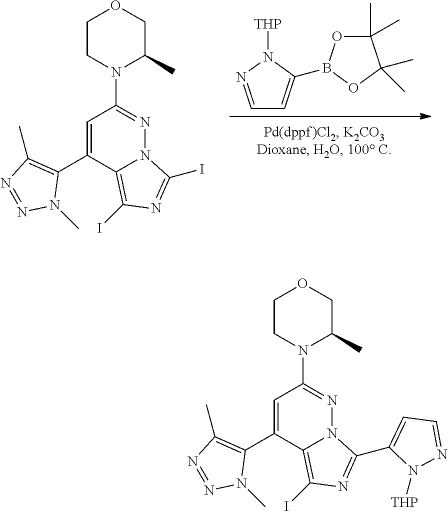

To a solution of (3R)-4-[4-(dimethyl-1H-1,2,3-triazol-5-yl)-5,7-diiodoimidazo[1,5-b]pyridazin-2-yl]-3-methylmorpholine (195.0 mg, 0.35 mmol) and 1-(oxan-2-yl)-5-(tetramethyl-1,3,2-dioxaborolan-2-yl)-1H-pyrazole (144.0 mg, 0.52 mmol) in co-solvent of dioxane (20 mL) and H₂O (2 mL) were added PdCl₂(PPh₃)₂ (48.4 mg, 0.07 mmol) and Cs₂CO₃ (337.3 mg, 1.04 mmol). The mixture was stirred at 100° C. overnight under N₂ atmosphere. LC-MS showed the reaction was complete. The reaction mixture was diluted with EA (50 mL), then washed with water and brine, dried over anhydrous Na₂SO₄, filtered and concentrated. The residue was purified by column chromatography on silica gel (DCM:MeOH=10:1, V/V) to give the desired product (66 mg, yield: 32%). LC/MS (ESI): m/z 590 [M+H]⁺.

### Step 6. (3R)-4-[4-(dimethyl-1H-1,2,3-triazol-5-yl)-7-(1H-pyrazol-5-yl)imidazo[1,5-b]pyridazin-2-yl]-3-methylmorpholine

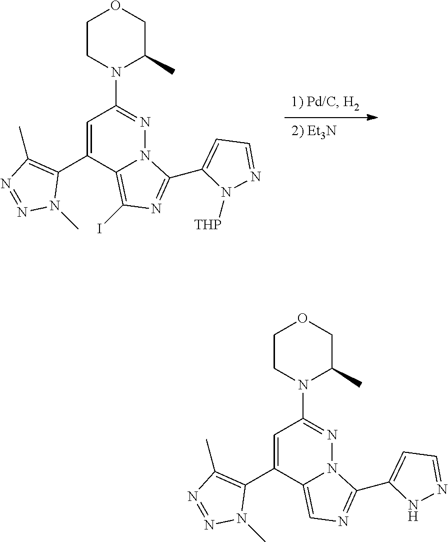

To a solution of (3R)-4-[4-(dimethyl-1H-1,2,3-triazol-5-yl)-5-iodo-7-[1-(oxan-2-yl)-1H-pyrazol-5-yl]imidazo[1,5-b]pyridazin-2-yl]-3-methylmorpholine (66 mg, 0.11 mmol) in MeOH (8 mL) was added Pd/C (10%, 10 mg). The mixture was stirred at room temperature overnight under hydrogen atmosphere. A drop of Et₃N was added to the above solution, then the resulting mixture was continued to stir at room temperature for an additional 2 h under H₂ atmosphere. LC-MS showed the reaction was complete. The reaction mixture was filtered, then concentrated. The residue was purified by prep-HPLC (C₁₈, 10-95%, MeOH in H₂O with 0.1% HCOOH) to give the desired product (5.8 mg, yield: 13%). LC/MS ESI (m/z): 380 [M+H]⁺. ¹H NMR (400 MHz, DMSO) δ 13.41 (br, 1H), 7.73 (s, 1H), 7.32 (s, 1H), 7.14 (d, J=1.9 Hz, 1H), 7.06 (s, 1H), 4.37 (d, J=6.4 Hz, 1H), 4.04-3.99 (m, 4H), 3.93 (d, J=12.1 Hz, 1H), 3.78 (d, J=11.4 Hz, 1H), 3.72 (dd, J=11.4, 2.6 Hz, 1H), 3.56 (dd, J=11.8, 2.8 Hz, 1H), 3.28-3.25 (m, 1H), 2.27 (s, 3H), 1.26 (d, J=6.7 Hz, 3H).

### Example 34

### Synthesis of (R)-3-methyl-4-(4-(1-methyl-1H-pyrazol-5-yl)-7-(3-methyl-1H-pyrazol-5-yl)imidazo[1,5-b]pyridazin-2-yl)morpholine

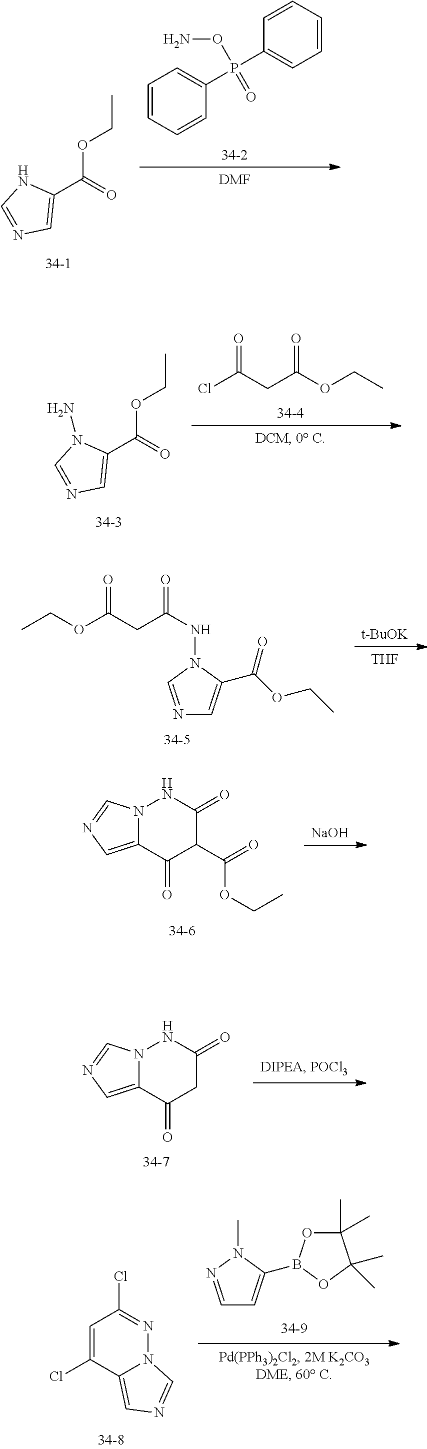

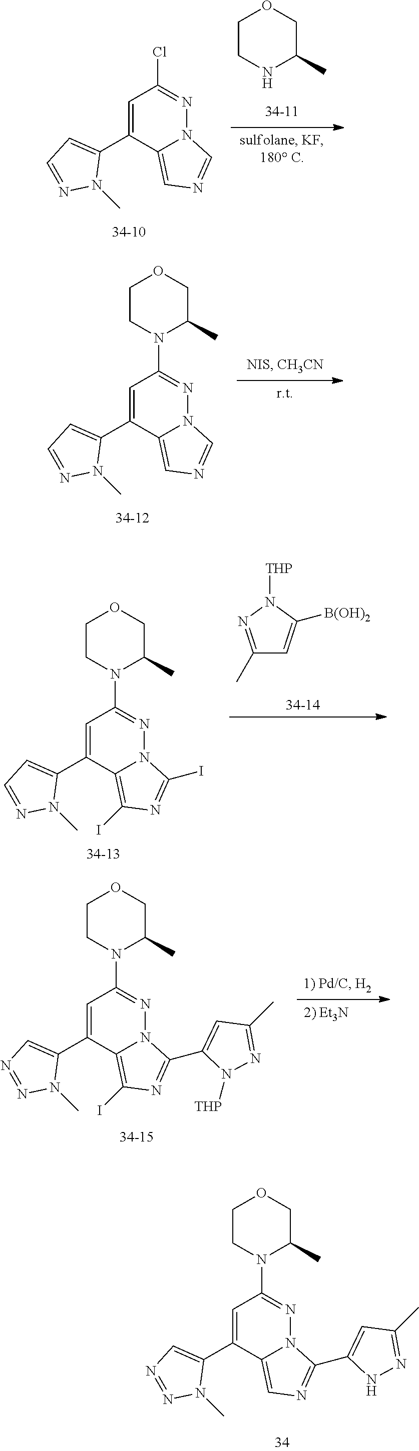

### Step 1. ethyl 1-amino-1H-imidazole-5-carboxylate

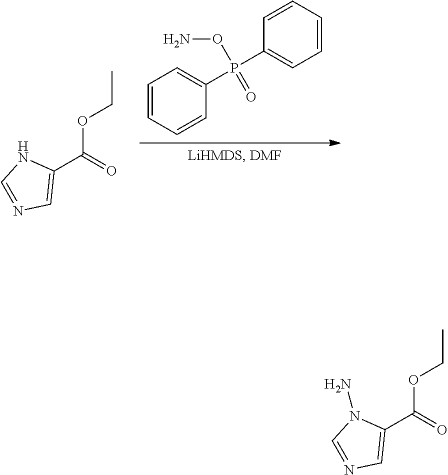

To a solution of ethyl 1H-imidazole-5-carboxylate (25 g, 178 mmol) in DMF (200 mL) at 0° C. was added LiHMDS (1M in THF, 196 mL, 196 mmol) drop wise. The mixture was stirred at 0° C. for 1 h, then amino diphenylphosphinate (50 g, 214 mmol) was added portion wise. After the addition, the resulting mixture was stirred at 0° C. for an additional 2 h. LC-MS showed the reaction was complete. The reaction mixture was quenched with H₂O (200 mL), then concentrated to dryness. The residue was diluted with EA (500 mL), then filtered. The filter cake was washed with EA (200 mL). The combined organic phase was dried over anhydrous Na₂SO₄, filtered and concentrated. The residue was purified by column chromatography (DCM:MeOH=10:1, V/V) to give the desired product (14 g, yield: 50.6%). LC/MS (ESI): m/z 156.2 [M+H]⁺.

### Step 2. ethyl 1-(3-ethoxy-3-oxopropanamido)-1H-imidazole-5-carboxylate

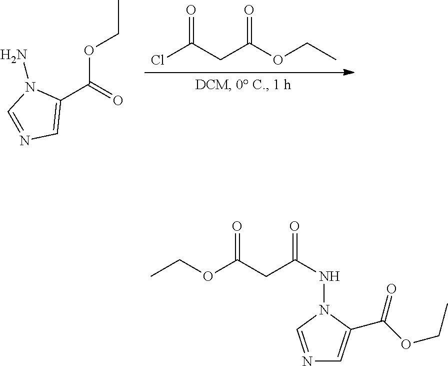

To a solution of ethyl 1-amino-1H-imidazole-5-carboxylate (14 g, 90.2 mmol) in DCM (200 mL) at 0° C. was added ethyl 3-chloro-3-oxopropanoate (15.1 mL, 117 mmol) drop wise. The mixture was stirred at room temperature for 16 h. LC-MS showed the reaction was complete. The reaction mixture was quenched with saturated NaHCO₃ aqueous solution, then extracted with DCM (100 mL×3). The combined organic phase was washed with brine, dried over anhydrous Na₂SO₄, filtered and concentrated to dryness. The residue was purified by column chromatography (DCM:MeOH=10:1, V/V) to give the desired product (24 g, yield: 98%). LC/MS (ESI): m/z 270.3 [M+H]⁺.

### Step 3. ethyl 2-hydroxy-4-oxo-3,4-dihydroimidazo[1,5-b]pyridazine-3-carboxylate

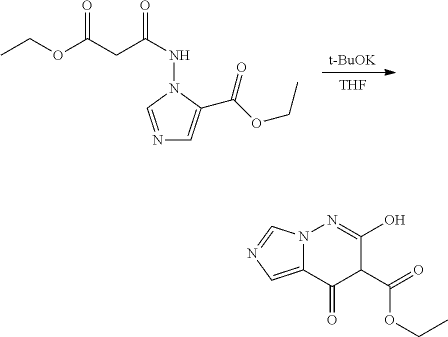 [SMILES: "t-BuOK" ; CCOC(=O)CC(=O)Nn1cncc1C(=O)OCC ; "THF" ; CCOC(=O)C1C(=O)c2cncn2N=C1O]

To a suspension of ethyl 1-(3-ethoxy-3-oxopropanamido)-1H-imidazole-5-carboxylate (24 g, 89.1 mmol) in THF (300 mL) at 0° C. was added t-BuOK (30 g, 267.0 mmol) portion wise. After the addition, the mixture was stirred at room temperature for 5 h. LC-MS showed the reaction was complete. The reaction mixture was adjusted to PH=2 by the addition of 6M HCl aqueous solution, then concentrated to dryness. The residue was suspended in co-solvent of DCM and MeOH (2:1, V:V, 200 mL), then stirred at room temperature for 0.5 h. The resulting mixture was filtered, the filter cake was washed with DCM and MeOH (2:1, V/V, 100 mL). The filtrate was concentrated under reduced pressure to give the crude product, which was used in the next step without further purification (16 g). LC/MS (ESI): m/z 224.2 [M+H]⁺.

### Step 4. imidazo[1,5-b]pyridazine-2,4 (1H,3H)-dione

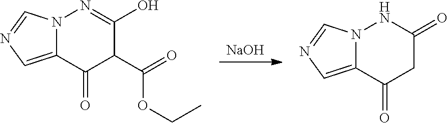 [SMILES: CCOC(=O)C1C(=O)c2cncn2N=C1O ; "NaOH" ; O=C1CC(=O)c2cncn2N1]

A mixture of ethyl 2-hydroxy-4-oxo-3,4-dihydroimidazo[1,5-b]pyridazine-3-carboxylate (16 g, 71.7 mmol) in NaOH aqueous solution (4M, 120 mL) was stirred at 100° C. for 16 h. LC-MS showed the reaction was complete. After cooling to room temperature, the mixture was adjusted to PH=2 by the addition of 6M HCl aqueous solution, then filtered. The filter cake was washed with ice-water twice (50 mL×2), then concentrated under vacuo to give the desired product (8 g, yield: 59%). LC/MS (ESI): m/z 152 [M+H]⁺.

### Step 5. 2,4-dichloroimidazo[1,5-b]pyridazine

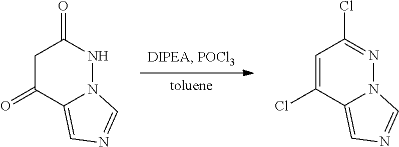 [SMILES: O=C1CC(=O)c2cncn2N1 ; "DIPEA, POCl3" ; "toluene" ; Clc1cc(Cl)c2cncn2n1]

To a solution of imidazo[1,5-b]pyridazine-2,4 (1H,3H)-dione (8 g, 52.9 mmol) and DIPEA (13.66 g, 106 mmol) in toluene (80 mL) at 0° C. was added POCl₃ (19.7 mL, 212 mmol) drop wise. After the addition, the mixture was stirred at 120° C. for 16 h. LC-MS showed the reaction was complete. The reaction mixture was concentrated, then diluted with EA (200 mL). The organic phase was washed with saturated NaHCO₃ aqueous solution and brine, dried over anhydrous Na₂SO₄, filtered and concentrated. The residue was purified by column chromatography (PE:EA=3:1, V/V) to give the desired product (7.2 g, yield: 72%). LC/MS (ESI): m/z 188/190 [M+H]⁺.

### Step 6. 2-chloro-4-(1-methyl-1H-pyrazol-5-yl)imidazo[1,5-b]pyridazine

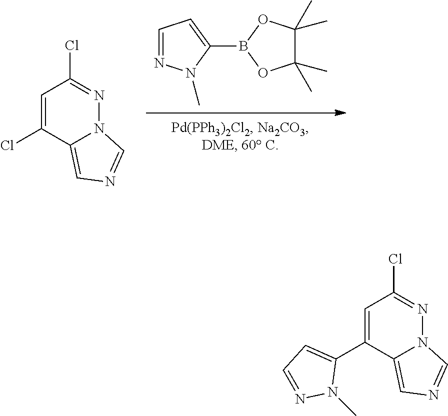 [SMILES: Clc1cc(Cl)c2cncn2n1 ; Cn1nccc1B1OC(C)(C)C(C)(C)O1 ; Cn1nccc1-c1cc(Cl)nn2cncc12]

To a solution of 2,4-dichloroimidazo[1,5-b]pyridazine (1 g, 5.32 mmol) and 1-methyl-5-(4,4,5,5-tetramethyl-1,3,2-dioxaborolan-2-yl)-1H-pyrazole (1.44 g, 6.91 mmol) in DME (20 mL) were added bis(triphenylphosphine)palladium(II) chloride (0.83 g, 1.06 mmol) and Na₂CO₃ (2M in H₂O, 5.32 mL, 10.64 mmol). The reaction was charged with N₂ twice, then stirred at 60° C. overnight. LC-MS showed the reaction was complete. The mixture was diluted with EA (50 mL), then washed with water and brine, dried over anhydrous Na₂SO₄, filtered and concentrated. The residue was purified by column chromatography on silica gel (PE:EA=3:1, V/V) to give the desired product (500 mg, yield: 40%). LC/MS ESI (m/z): 234 [M+H]⁺.

### Step 7. (R)-3-methyl-4-(4-(1-methyl-1H-pyrazol-5-yl)imidazo[1,5-b]pyridazin-2-yl)morpholine

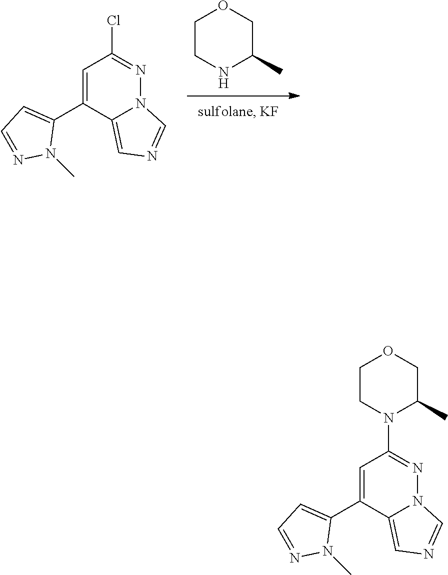

To a solution of 2-chloro-4-(1-methyl-1H-pyrazol-5-yl)imidazo[1,5-b]pyridazine (1 g, 4.28 mmol) in sulfolane (20 mL) was added (R)-3-methylmorpholine (1.30 g, 12.839 mmol) and KF (0.75 g, 12.839 mmol). The mixture was stirred at 180° C. for 8 h. LC-MS showed the reaction was complete. The mixture was diluted with EA (50 mL), then washed with water and brine, dried over anhydrous Na₂SO₄, filtered and concentrated. The residue was purified by column chromatography on silica gel (DCM:MeOH=20:1, V/V) to give the desired product (330 mg, yield: 26%). LC/MS ESI (m/z): 299 [M+H]⁺.

### Step 8. (3R)-4-[5,7-diiodo-4-(1-methyl-1H-pyrazol-5-yl)imidazo[1,5-b]pyridazin-2-yl]-3-methylmorpholine

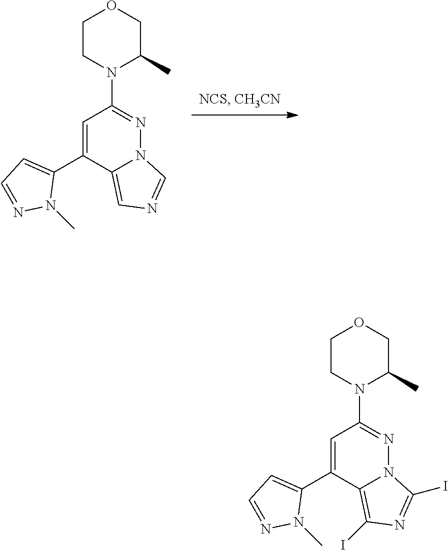

To a solution of (3R)-3-methyl-4-[4-(1-methyl-1H-pyrazol-5-yl)imidazo[1,5-b]pyridazin-2-yl]morpholine (230 mg, 0.77 mmol) in MeCN (15 mL) was added NIS (520.3 mg, 2.31 mmol). The mixture was stirred at room temperature for 2 h. LC-MS showed the reaction was complete. The mixture was diluted with EA (50 mL), then washed with water and brine, dried over anhydrous Na₂SO₄, filtered and concentrated. The residue was purified by column chromatography on silica gel (DCM:MeOH=20:1, V/V) to give the desired product (340 mg, yield: 80%). LC/MS ESI (m/z): 551 [M+H]⁺.

### Step 10. (3R)-4-(5-iodo-7-(3-methyl-1-(tetrahydro-2H-pyran-2-yl)-1H-pyrazol-5-yl)-4-(1-methyl-1H-pyrazol-5-yl)imidazo[1,5-b]pyridazin-2-yl)-3-methylmorpholine

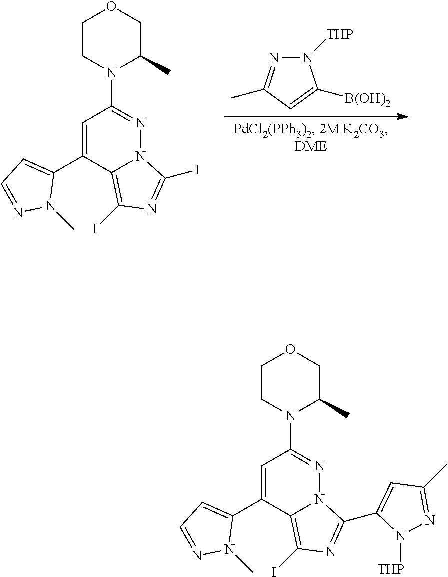

To a solution of (3R)-4-[5,7-diiodo-4-(1-methyl-1H-pyrazol-5-yl)imidazo[1,5-b] pyridazin-2-yl]-3-methylmorpholine (200 mg, 0.36 mmol) and [3-methyl-1-(oxan-2-yl)-1H-pyrazol-5-yl]boronic acid (152 mg, 0.72 mmol) in DME (6 mL) were added PdCl₂(PPh₃)₂ (51 mg, 0.07 mmol) and K₂CO₃ (2.0 M in H₂O, 0.45 mL, 0.90 mmol). The mixture was stirred at 100° C. for 16 h under N₂ atmosphere.

LC-MS showed the reaction was complete. The reaction mixture was diluted with EA (40 mL), then washed with water and brine, dried over anhydrous Na₂SO₄, filtered and concentrated. The residue was purified by column chromatography on silica gel (DCM:MeOH=20:1, V/V) to afford the desired product (134 mg, yield: 62%).

LC/MS(ESI): m/z 589 [M+H]⁺.

### Step 11. (R)-3-methyl-4-(4-(1-methyl-1H-pyrazol-5-yl)-7-(3-methyl-1H-pyrazol-5-yl)imidazo[1,5-b]pyridazin-2-yl)morpholine

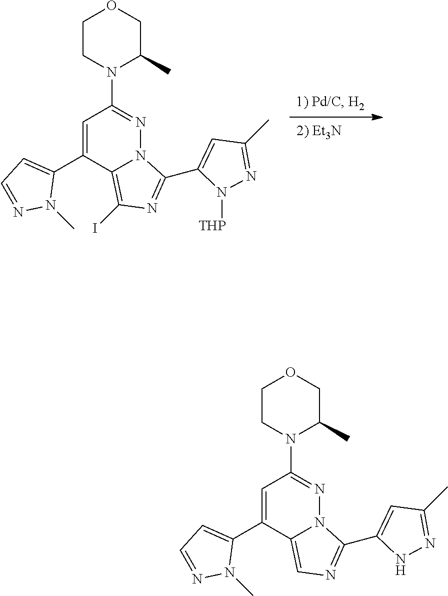

To a solution of (3R)-4-{5-iodo-7-[3-methyl-1-(oxan-2-yl)-1H-pyrazol-5-yl]-4-(1-methyl-1H-pyrazol-5-yl)imidazo[1,5-b]pyridazin-2-yl}-3-methylmorpholine (134 mg, 0.22 mmol) in MeOH (3 mL) was Pd/C (10%, 20 mg). The mixture was stirred at room temperature for 12 h under H₂ atmosphere. A drop of Et₃N was added to the above solution, then resulting mixture was continued to stir at room temperature for an additional 2 h under H₂ atmosphere. LC-MS showed the reaction was complete. The reaction mixture was filtered, then concentrated. The residue was purified by Prep-HPLC (C18, 10-95%, MeOH in H₂O with 0.1% HCOOH) to give the desired product (10 mg, yield: 11%). LC/MS (ESI): m/z 379 [M+H]⁺. ¹H NMR (400 MHz, DMSO) δ 12.95 (s, 1H), 7.65 (d, J=1.9 Hz, 1H), 7.41 (s, 1H), 6.96 (s, 1H), 6.88 (s, 1H), 6.80 (d, J=1.9 Hz, 1H), 4.39 (d, J=6.6 Hz, 1H), 4.04-4.00 (m, 1H), 3.98 (s, 3H), 3.92 (d, J=12.0 Hz, 1H), 3.75 (dt, J=11.5, 7.0 Hz, 2H), 3.58 (td, J=11.8, 2.8 Hz, 1H), 3.30-3.22 (m, 1H), 2.29 (s, 3H), 1.26 (d, J=6.7 Hz, 3H).

### Example 35

### Synthesis of (3R)-4-(4-(1,4-dimethyl-1H-1,2,3-triazol-5-yl)-7-(3-methyl-1H-pyrazol-5-yl)imidazo[1,5-b]pyridazin-2-yl)-3-methylmorpholine

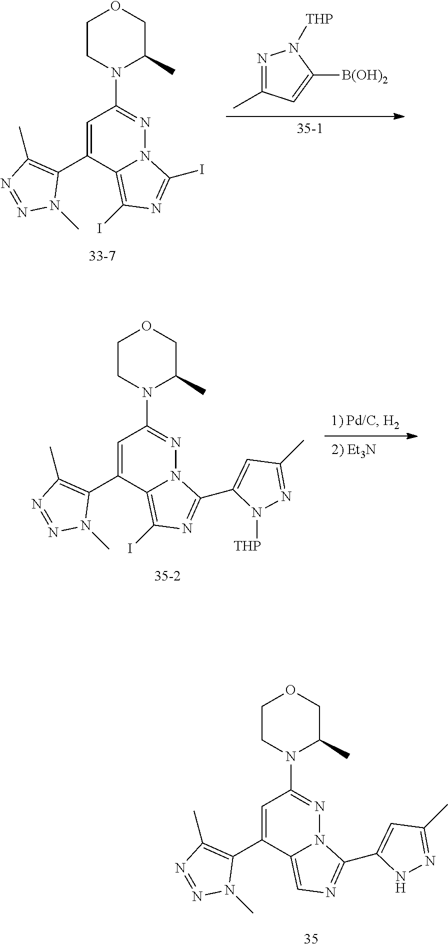

### Step 1. (3R)-4-[4-(dimethyl-1H-1,2,3-triazol-5-yl)-5-iodo-7-[3-methyl-1-(oxan-2-yl)-1H-pyrazol-5-yl]imidazo[1,5-b]pyridazin-2-yl]-3-methylmorpholine

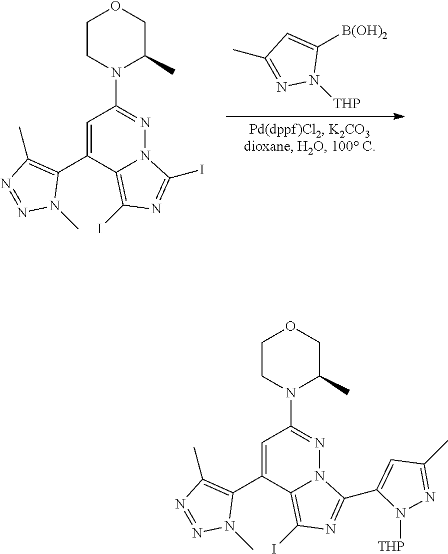

To a solution of (3R)-4-[4-(dimethyl-1H-1,2,3-triazol-5-yl)-5,7-diiodoimidazo[1,5-b] pyridazin-2-yl]-3-methylmorpholine (276 mg, 0.49 mmol) and [3-methyl-1-(oxan-2-yl)-1H-pyrazol-5-yl]boronic acid (307.7 mg, 1.47 mmol) in co-solvent of dioxane (20 mL) and H₂O (2 mL) were added PdCl₂(PPh₃)₂ (68.56 mg, 0.10 mmol) and Cs₂CO₃ (636.5 mg, 1.95 mmol). The mixture was stirred at 100° C. overnight under nitrogen atmosphere. LC-MS showed the reaction was complete.

The mixture was diluted with EA (50 mL), then washed with water and brine, dried over anhydrous Na₂SO₄, filtered and concentrated. The residue was purified by column chromatography on silica gel (DCM:MeOH=20:1, V/V) to give the desired product (82 mg, yield: 28%). LC/MS ESI (m/z): 604 [M+H]⁺.

### Step 2. (3R)-4-(4-(1,4-dimethyl-1H-1,2,3-triazol-5-yl)-7-(3-methyl-1-(tetrahydro-2H-pyran-2-yl)-1H-pyrazol-5-yl)imidazo[1,5-b]pyridazin-2-yl)-3-methylmorpholine

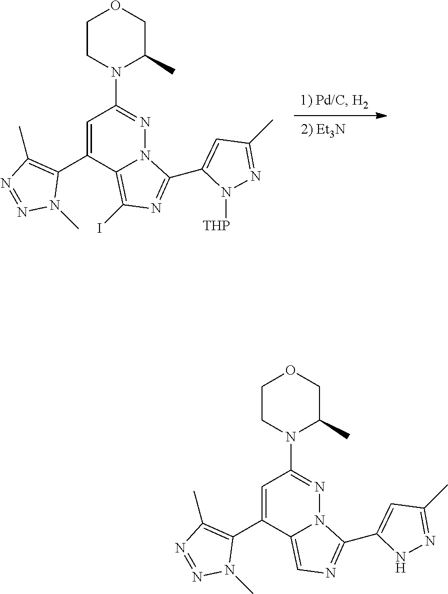

To a solution of (3R)-4-(4-(1,4-dimethyl-1H-1,2,3-triazol-5-yl)-5-iodo-7-(3-methyl-1-(tetrahydro-2H-pyran-2-yl)-1H-pyrazol-5-yl)imidazo[1,5-b]pyridazin-2-yl)-3-methylmorpholine (60 mg, 0.1 mmol) in MeOH (8 mL) was added Pd/C (10%, 6 mg). The mixture was stirred at room temperature for 12 h under H₂ atmosphere. A drop of Et₃N was added to the above solution, then the resulting mixture was continued to stir at room temperature for an additional 2 h under H₂ atmosphere. LC-MS showed the reaction was complete. The reaction mixture was filtered, then concentrated. The residue was purified by prep-HPLC (C₁₈, 10-95%, MeOH in H₂O with 0.1% HCOOH) to give the desired product (10 mg, yield: 25%). LC/MS ESI (m/z): 394 [M+H]⁺. ¹H NMR (400 MHz, DMSO) δ 7.29 (s, 1H), 7.04 (s, 1H), 6.88 (s, 1H), 4.35 (d, J=6.6 Hz, 1H), 4.05-3.98 (m, 4H), 3.93 (d, J=12.7 Hz, 1H), 3.74 (dt, J=11.6, 7.0 Hz, 2H), 3.57 (td, J=11.9, 2.8 Hz, 1H), 3.27 (dd, J=12.9, 3.6 Hz, 1H), 2.30 (s, 3H), 2.26 (s, 3H), 1.26 (d, J=6.7 Hz, 3H).

### Example 36

### Synthesis of (R)-3-methyl-4-(5-methyl-4-(1-methyl-1H-pyrazol-5-yl)-7-(1H-pyrazol-5-yl)imidazo[1,5-b]pyridazin-2-yl)morpholine

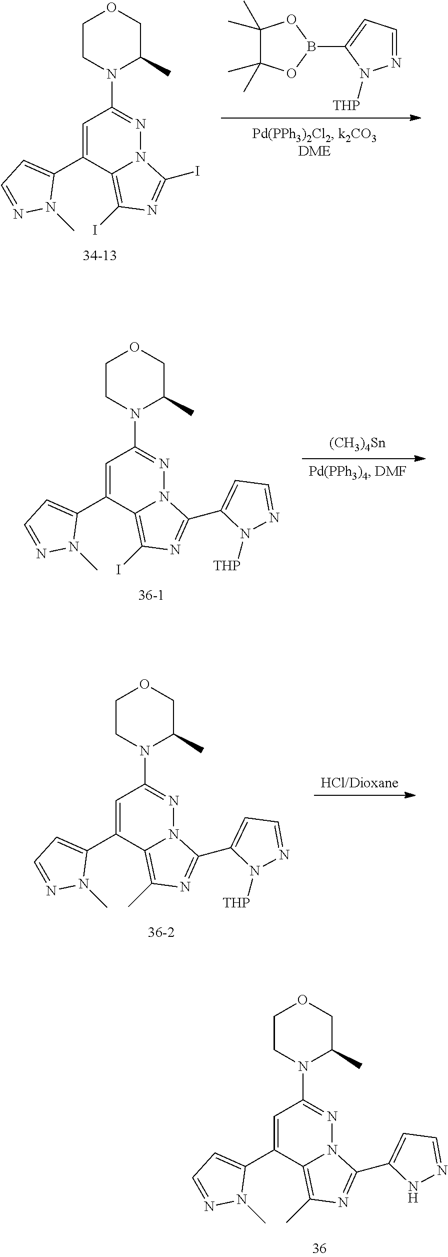

### Step 1. (3R)-4-(5-iodo-4-(1-methyl-1H-pyrazol-5-yl)-7-(1-(tetrahydro-2H-pyran-2-yl)-1H-pyrazol-5-yl)imidazo[1,5-b]pyridazin-2-yl)-3-methylmorpholine

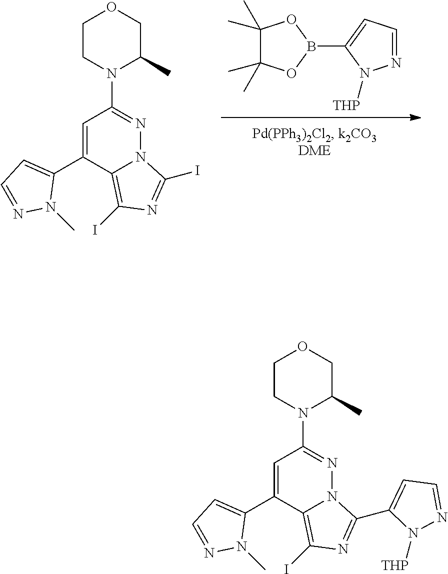

To a solution of (R)-4-(5,7-diiodo-4-(1-methyl-1H-pyrazol-5-yl)imidazo[1,5-b] pyridazin-2-yl)-3-methylmorpholine (300 mg, 0.55 mmol) and 1-(tetrahydro-2H-pyran-2-yl)-5-(4,4,5,5-tetramethyl-1,3,2-dioxaborolan-2-yl)-1H-pyrazole (303.4 mg, 1.09 mmol) in DME (8 mL) were added K₂CO₃ (2M in H₂O, 0.82 mL, 1.64 mmol) and Bis(triphenylphosphine)palladium(II) chloride (42.4 mg, 0.06 mmol). The mixture was stirred at 80° C. overnight under nitrogen atmosphere. LC-MS showed the reaction was complete. The mixture was diluted with DCM (50 mL), then washed with water and brine, dried over anhydrous Na₂SO₄, filtered and concentrated. The residue was purified by column chromatography on silica gel (PE:EA=3:1, V/V) to give the desired product (120 mg, yield: 38%). LC/MS ESI (m/z): 575 [M+H]⁺.

### Step 2. (3R)-3-methyl-4-(5-methyl-4-(1-methyl-1H-pyrazol-5-yl)-7-(1-(tetrahydro-2H-pyran-2-yl)-1H-pyrazol-5-yl)imidazo[1,5-b]pyridazin-2-yl)morpholine

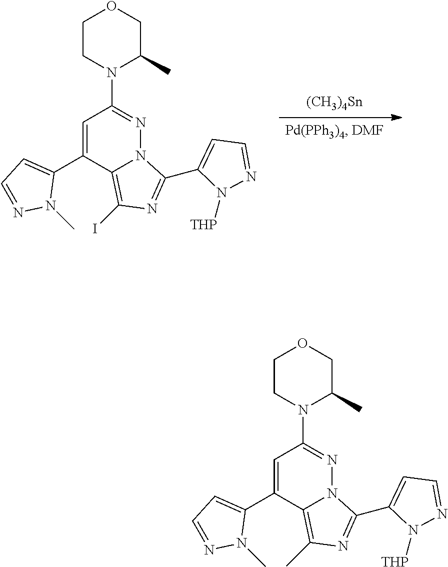

To a solution of (3R)-4-(5-iodo-4-(1-methyl-1H-pyrazol-5-yl)-7-(1-(tetrahydro-2H-pyran-2-yl)-1H-pyrazol-5-yl)imidazo[1,5-b]pyridazin-2-yl)-3-methylmorpholine (120 mg, 0.21 mmol) in DMF (3 mL) were added tetramethyltin (0.15 mL, 1.05 mmol) and Pd(PPh₃)₄ (24.1 mg, 0.02 mmol). The mixture was stirred at 100° C. overnight under nitrogen atmosphere. LC-MS showed the reaction was complete. The mixture was diluted with DCM (50 mL), then washed with water and brine, dried over anhydrous Na₂SO₄, filtered and concentrated. The residue was purified by column chromatography on silica gel (PE:EA=3:1, V/V) to give the desired product (80 mg, yield: 83%). LC/MS ESI (m/z): 463 [M+H]⁺.

### Step 3. (R)-3-methyl-4-(5-methyl-4-(1-methyl-1H-pyrazol-5-yl)-7-(1H-pyrazol-5-yl)imidazo[1,5-b]pyridazin-2-yl)morpholine

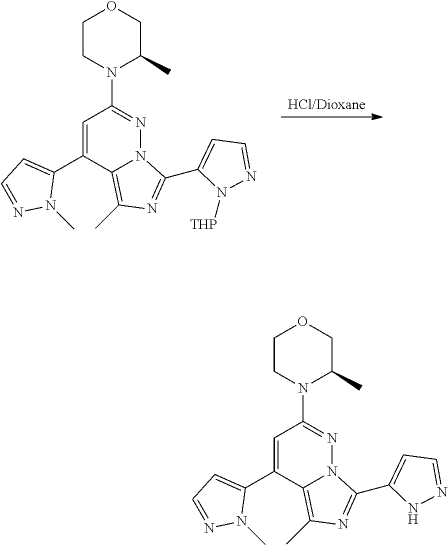

To a solution of (3R)-3-methyl-4-(5-methyl-4-(1-methyl-1H-pyrazol-5-yl)-7-(1-(tetra hydro-2H-pyran-2-yl)-1H-pyrazol-5-yl)imidazo[1,5-b]pyridazin-2-yl)morpholine (80 mg, 0.17 mmol) in DCM (2 mL) was added HCl solution (4M in Dioxane, 1 mL). The mixture was stirred at room temperature for 1 h. LC-MS showed the reaction was complete. The reaction mixture was concentrated under reduced pressure. The residue was purified by Prep-HPLC (C18, 10-95%, MeOH in H₂O with 0.1% HCOOH) to give the desired product (20 mg, yield: 31%). LC/MS (ESI) m/z: 379 [M+H]⁺. ¹H NMR (400 MHz, DMSO) δ 13.46 (s, 1H), 7.67 (s, 1H), 7.62 (d, J=1.9 Hz, 1H), 7.10 (d, J=1.7 Hz, 1H), 6.77 (s, 1H), 6.55 (d, J=1.9 Hz, 1H), 4.34 (d, J=6.6 Hz, 1H), 3.99 (dd, J=11.2, 3.2 Hz, 1H), 3.89 (d, J=13.2 Hz, 1H), 3.75 (d, J=9.7 Hz, 4H), 3.69 (dd, J=11.4, 2.7 Hz, 1H), 3.55 (td, J=11.8, 2.8 Hz, 1H), 3.25 (d, J=12.4 Hz, 1H), 1.93 (s, 3H), 1.24 (d, J=6.7 Hz, 3H).

### Example 37

### Synthesis of (R)-3-methyl-4-(7-(1-methyl-1H-pyrazol-5-yl)-3-(3-methyl-1H-pyrazol-5-yl)isothiazolo[4,5-b]pyridin-5-yl)morpholine

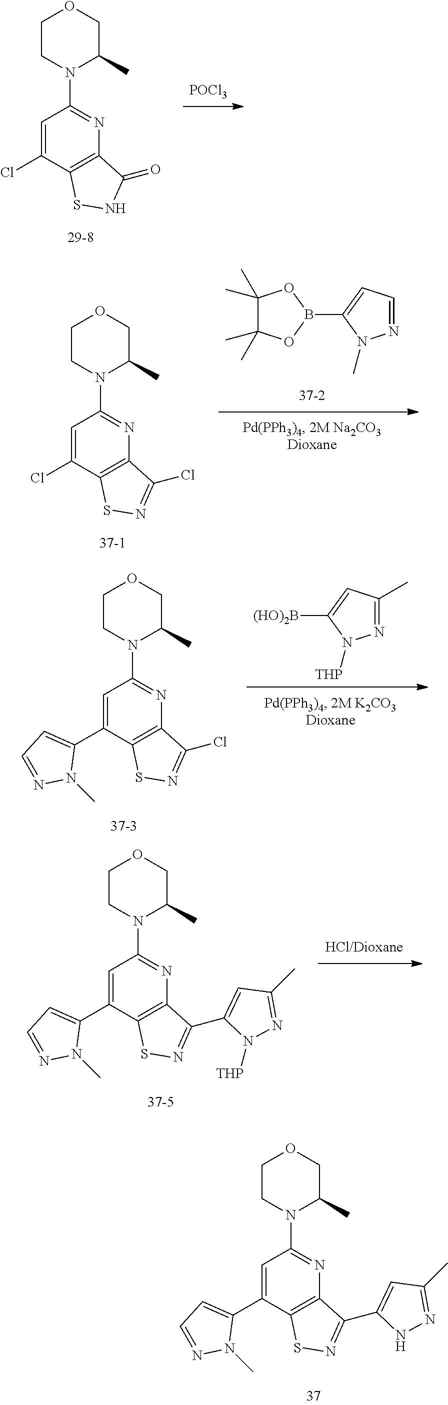

### Step 1. (R)-4-(3,7-dichloroisothiazolo[4,5-b]pyridin-5-yl)-3-methylmorpholine

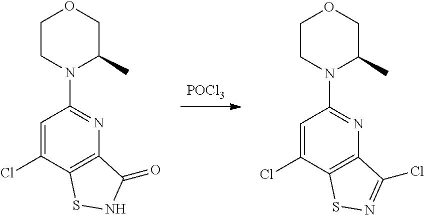 [SMILES: C[C@@H]1COCCN1c1cc(Cl)c2s[nH]c(=O)c2n1 ; "POCl3" ; C[C@@H]1COCCN1c1cc(Cl)c2snc(Cl)c2n1]

A mixture of 7-chloro-5-[(3R)-3-methylmorpholin-4-yl]-2H,3H-[1,2]thiazolo[4,5-b] pyridin-3-one (90 mg, 0.32 mmol) and POCl₃ (0.88 mL, 9.45 mmol) was stirred at 100° C. for 12 h. LC-MS showed the reaction was complete.

After cooling to room temperature, the mixture was diluted with DCM (30 mL), then poured into ice-water. The organic layer was separated, then washed with saturated NaHCO₃ aqueous solution and brine, dried over anhydrous Na₂SO₄, filtered and concentrated. The residue was purified by column chromatography on silica gel (PE:EA=10:1) to give the desired product (60 mg, yield: 63%). LC/MS ESI (m/z): 304/306 [M+H]⁺.

### Step 2. (R)-4-(3-chloro-7-(1-methyl-1H-pyrazol-5-yl)isothiazolo[4,5-b]pyridin-5-yl)-3-methylmorpholine

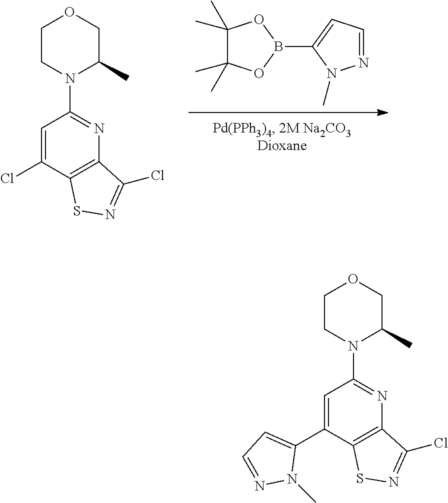 [SMILES: C[C@@H]1COCCN1c1cc(Cl)c2snc(Cl)c2n1 ; Cn1nccc1B1OC(C)(C)C(C)(C)O1 ; C[C@@H]1COCCN1c1cc(-c2ccnn2C)c2snc(Cl)c2n1]

To a solution of (3R)-4-{3,7-dichloro-[1,2]thiazolo[4,5-b]pyridin-5-yl}-3-methyl morpholine (50 mg, 0.16 mmol) and 1-methyl-5-(tetramethyl-1,3,2-dioxaborolan-2-yl)-1H-pyrazole (68.4 mg, 0.33 mmol) in dioxane (2 mL) were added Pd(PPh₃)₄ (38.0 mg, 0.03 mmol) and Na₂CO₃ (2M in H₂O, 0.16 mL, 0.33 mmol). The mixture was charged with N₂ twice, then stirred at room temperature for 12 h. LC-MS showed the reaction was complete. After cooling to room temperature, the mixture was diluted with DCM (30 mL), then washed with water and brine, dried over anhydrous Na₂SO₄, filtered and concentrated. The residue was purified by column chromatography on silica gel (PE:EA=10:1) to give the desired product (10 mg, yield: 17%). LC/MS ESI (m/z): 350 [M+H]⁺.

### Step 3. (3R)-3-methyl-4-(3-(3-methyl-1-(tetrahydro-2H-pyran-2-yl)-1H-pyrazol-5-yl)-7-(1-methyl-1H-pyrazol-5-yl)isothiazolo[4,5-b]pyridin-5-yl)morpholine

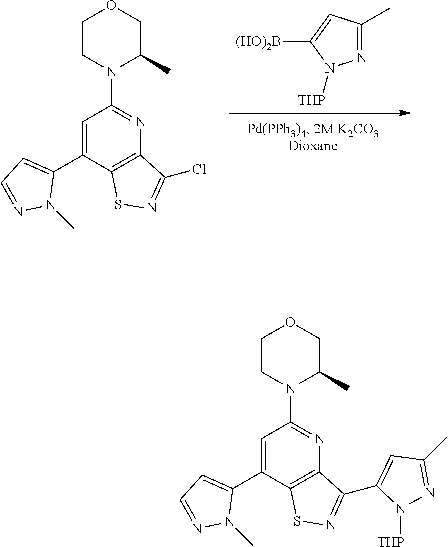

To a solution of (3R)-4-[3-chloro-7-(1-methyl-1H-pyrazol-5-yl)-[1,2]thiazolo[4,5-b] pyridin-5-yl]-3-methylmorpholine (10 mg, 0.03 mmol) and 3-methyl-1-(oxan-2-yl)-5-(tetramethyl-1,3,2-dioxaborolan-2-yl)-1H-pyrazole (16.7 mg, 0.06 mmol) in dioxane (2 mL) were added Pd(PPh₃)₄ (3.30 mg, 0.003 mmol) and K₂CO₃ (2M in H₂O, 0.03 mL, 0.06 mmol). The mixture was charged with N₂ twice, then stirred at 100° C. for 12 h. LC-MS showed the reaction was complete. After cooling to room temperature, the mixture was diluted with DCM (30 mL), then washed with water and brine, dried over anhydrous Na₂SO₄, filtered and concentrated. The residue was purified by column chromatography on silica gel (PE:EA=10:1, V/V) to give the desired product (3 mg, yield: 22%). LC/MS ESI (m/z): 480 [M+H]⁺.

### Step 4. (R)-3-methyl-4-(7-(1-methyl-1H-pyrazol-5-yl)-3-(3-methyl-1H-pyrazol-5-yl)isothiazolo[4,5-b]pyridin-5-yl)morpholine

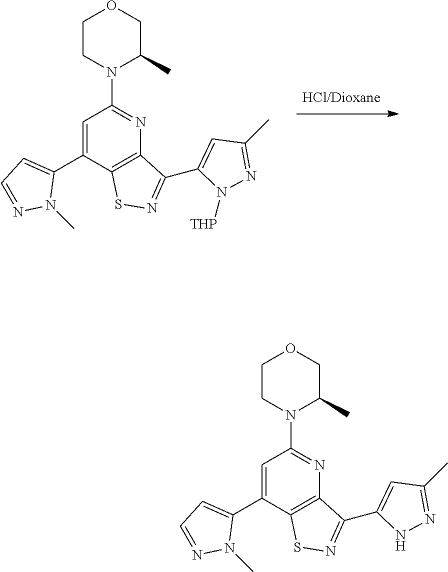

To a solution of (3R)-3-methyl-4-{3-[3-methyl-1-(oxan-2-yl)-1H-pyrazol-5-yl]-7-(1-methyl-1H-pyrazol-5-yl)-[1,2]thiazolo[4,5-b]pyridin-5-yl}morpholine (3 mg, 0.006 mmol) in DCM (1 mL) was added HCl solution (4M in dioxane, 1 mL). The resulting mixture was stirred at room temperature for 2 h. LC-MS showed the reaction was complete. The mixture was concentrated under reduced pressure, the residue was purified by prep-HPLC (C₁₈, 10-95%, MeOH in H₂O with 0.1% HCOOH) to give the desired product (1 mg, yield: 40%). LC/MS ESI (m/z): 396 [M+H]⁺. ¹H NMR (400 MHz, DMSO) δ 7.67 (d, J=2.0 Hz, 1H), 7.40 (s, 1H), 7.15 (s, 1H), 6.77 (d, J=2.0 Hz, 1H), 4.57 (d, J=6.3 Hz, 1H), 4.19 (d, J=12.6 Hz, 1H), 4.05 (d, J=8.0 Hz, 1H), 3.99 (s, 3H), 3.82 (d, J=11.3 Hz, 1H), 3.73 (dd, J=11.5, 2.8 Hz, 1H), 3.59 (dd, J=11.6, 8.9 Hz, 2H), 3.24 (d, J=3.5 Hz, 1H), 2.32 (s, 3H), 1.26 (d, J=6.6 Hz, 4H).

### Example 38

### Synthesis of (R)-4-(7-(1,4-dimethyl-1H-1,2,3-triazol-5-yl)-3-(1H-pyrazol-5-yl)isothiazolo[4,5-b]pyridin-5-yl)-3-methylmorpholine

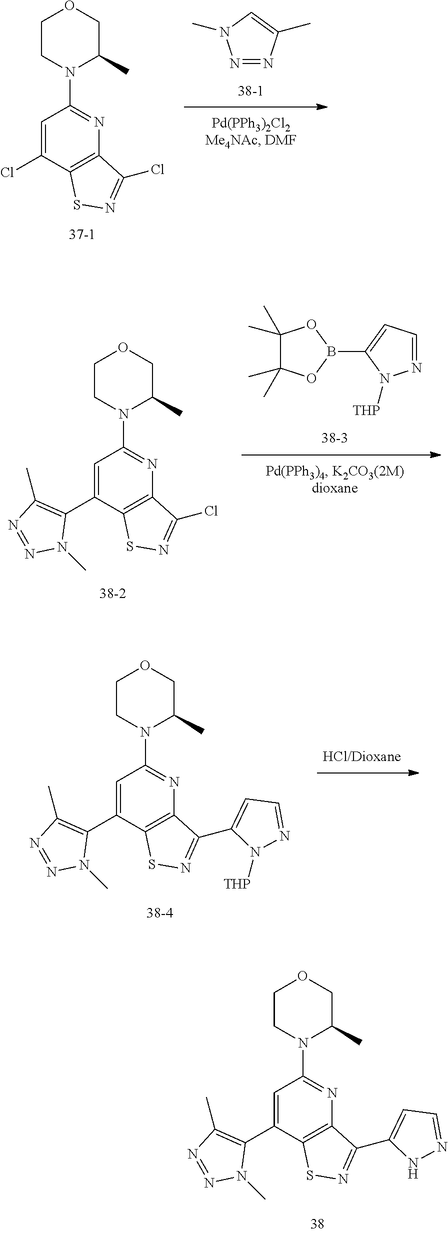

### Step 1. (R)-4-(3-chloro-7-(1,4-dimethyl-1H-1,2,3-triazol-5-yl)isothiazolo[4,5-b]pyridin-5-yl)-3-methylmorpholine

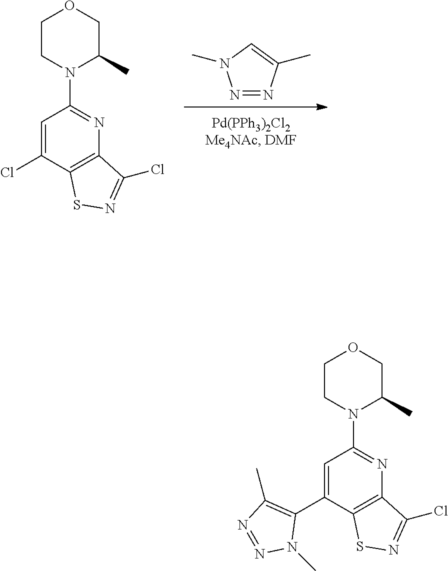

To a solution of (3R)-4-{3,7-dichloro-[1,2]thiazolo[4,5-b]pyridin-5-yl}-3-methyl morpholine (70 mg, 0.23 mmol), 1,4-dimethyl-1H-1,2,3-triazole (112 mg, 1.15 mmol) and Me₄NAc (81 mg, 0.69 mmol) in DMF (3 mL) was added Pd(PPh₃)₂Cl₂ (32 mg, 0.05 mmol). The mixture was stirred at 140° C. for 6 h. LC-MS showed the reaction was complete. The mixture was diluted with DCM (50 mL), then washed with water and brine, dried over anhydrous Na₂SO₄, filtered and concentrated. The residue was purified by column chromatography on silica gel (PE:EA=3:1, V/V) to give the desired product (45 mg, yield: 54%). LC/MS ESI (m/z): 365 [M+H]⁺.

### Step 2. (3R)-4-(7-(1,4-dimethyl-1H-1,2,3-triazol-5-yl)-3-(1-(tetrahydro-2H-pyran-2-yl)-1H-pyrazol-5-yl)isothiazolo[4,5-b]pyridin-5-yl)-3-methylmorpholine

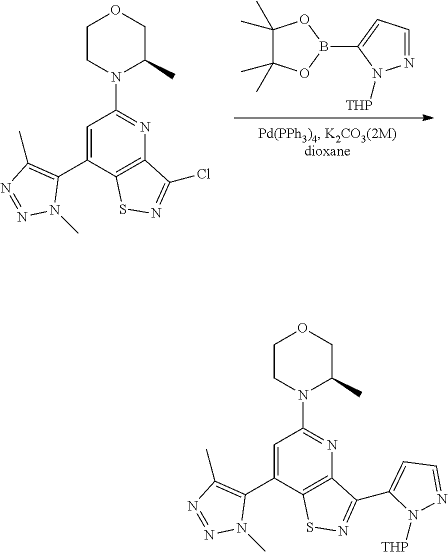

To a solution of (R)-4-(3-chloro-7-(1,4-dimethyl-1H-1,2,3-triazol-5-yl)isothiazolo[4,5-b]pyridin-5-yl)-3-methylmorpholine (45 mg, 0.12 mmol), 1-(oxan-2-yl)-5-(tetra methyl-1,3,2-dioxaborolan-2-yl)-1H-pyrazole (69 mg, 0.25 mmol) and K₂CO₃ (2M in H₂O, 0.19 mL, 0.37 mmol) in dioxane (3 mL) was added Pd(PPh₃)₄ (14 mg, 0.01 mmol). The mixture was stirred at 100° C. for 16 h. LC-MS showed the reaction was complete. The mixture was diluted with DCM (50 mL), then washed with water and brine, dried over anhydrous Na₂SO₄, filtered and concentrated. The residue was purified by column chromatography on silica gel (DCM:MeOH=20:1, V/V) to give the desired product (22 mg, yield: 37%). LC/MS ESI (m/z): 481 [M+H]⁺.

### Step 3. (R)-4-(7-(1,4-dimethyl-1H-1,2,3-triazol-5-yl)-3-(1H-pyrazol-5-yl)isothiazolo [4,5-b]pyridin-5-yl)-3-methylmorpholine

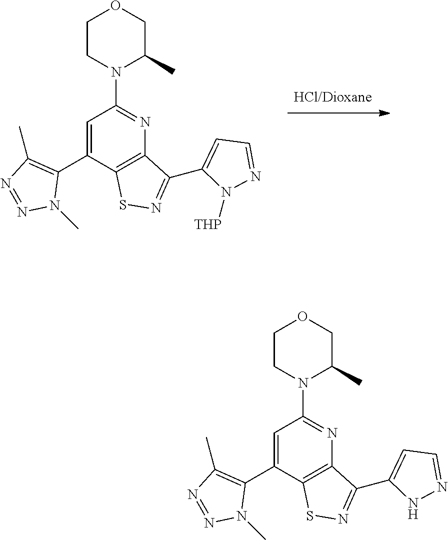

To a solution of (3R)-4-(7-(1,4-dimethyl-1H-1,2,3-triazol-5-yl)-3-(1-(tetrahydro-2H-pyran-2-yl)-1H-pyrazol-5-yl)isothiazolo[4,5-b]pyridin-5-yl)-3-methylmorpholine (22 mg, 0.05 mmol) in DCM (2 mL) was added HCl solution (4M in dioxane, 1 mL). The mixture was stirred at room temperature for 1 h. LC-MS showed the reaction was complete. The reaction mixture was concentrated under reduced pressure. The residue was purified by Prep-HPLC (C18, 10-95%, MeOH in H₂O with 0.1% HCOOH) to give the desired product (2.5 mg, yield: 14%). LC/MS (ESI) m/z: 397.5 [M+H]⁺. ¹H NMR (400 MHz, DMSO) δ 13.53 (d, J=176.0 Hz, 1H), 7.82 (d, J=88.5 Hz, 1H), 7.48 (s, 1H), 7.44 (d, J=1.3 Hz, 1H), 4.56 (dd, J=11.6, 6.3 Hz, 1H), 4.20 (dt, J=13.4, 5.9 Hz, 1H), 4.05 (dd, J=12.1, 2.6 Hz, 1H), 3.99 (s, 3H), 3.82 (d, J=11.5 Hz, 1H), 3.72 (dd, J=11.5, 2.7 Hz, 1H), 3.62-3.54 (m, 1H), 3.28-3.23 (m, 1H), 2.25 (s, 3H), 1.27 (d, J=6.7 Hz, 3H).

### Example 39

### Synthesis of (3R)-4-[4-(diethylphosphoryl)-7-(1H-pyrazol-5-yl)imidazo[1,5-b]pyridazin-2-yl]-3-methylmorpholine

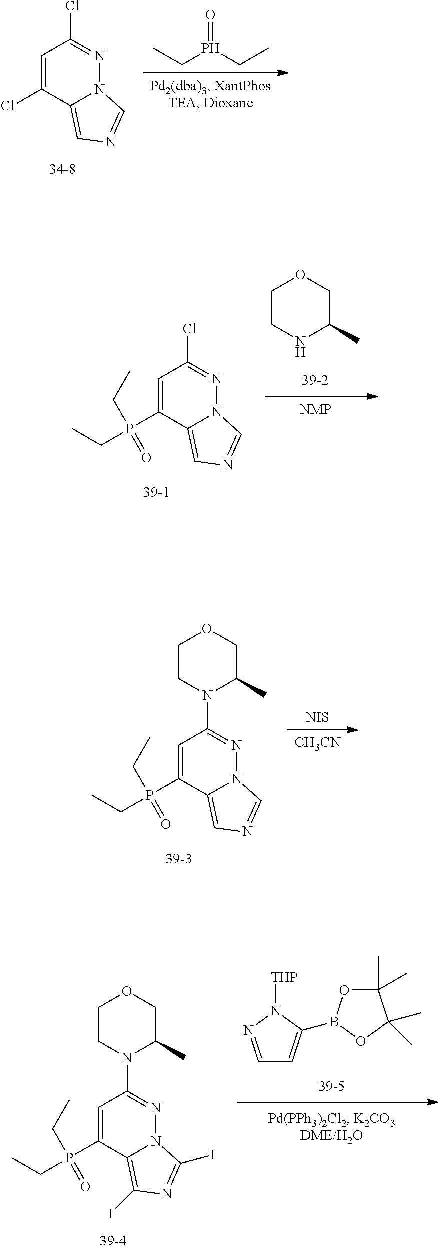

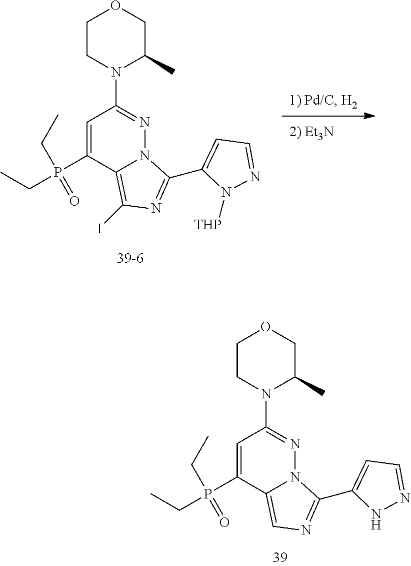

### Step 1. 2-chloro-4-(diethylphosphoryl)imidazo[1,5-b]pyridazine

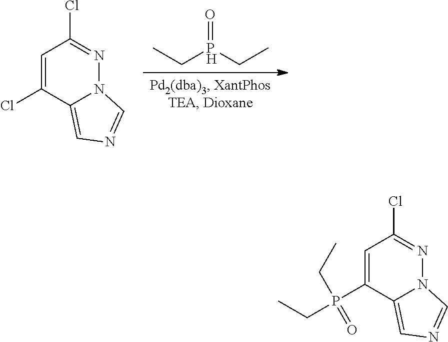

To a solution of 2,4-dichloroimidazo[1,5-b]pyridazine (500 mg, 2.66 mmol) and (ethylphosphonoyl)ethane (338.6 mg, 3.19 mmol) in dioxane (15 mL) were added Pd(dba)₃ (243.5 mg, 0.27 mmol), XantPhos (153.9 mg, 0.27 mmol) and TEA (0.74 mL, 5.34 mmol). The mixture was stirred at 70° C. overnight under nitrogen atmosphere. LC-MS showed the reaction was complete. The reaction mixture was diluted with DCM (20 mL), then washed with water and brine, dried over anhydrous Na₂SO₄, filtered and concentrated. The residue was purified by column chromatography on silica gel (DCM:MeOH=20:1, V/V) to afford the desired product (560 mg, yield: 82%). LC/MS(ESI): m/z 258 [M+H]⁺.

### Step 2. (3R)-4-[4-(diethylphosphoryl)imidazo[1,5-b]pyridazin-2-yl]-3-methylmorpholine

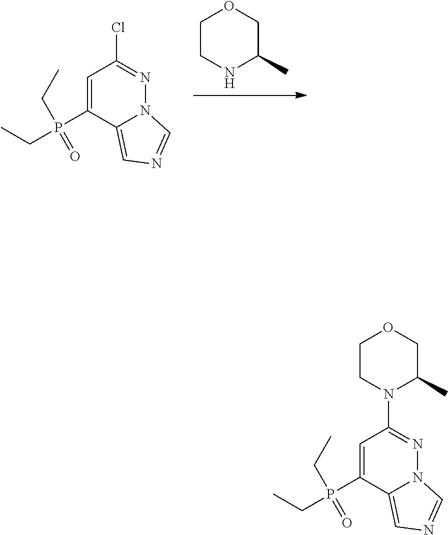

To a solution of 2-chloro-4-(diethylphosphoryl)imidazo[1,5-b]pyridazine (560 mg, 2.17 mmol) in NMP (15 mL) was added (3R)-3-methylmorpholine (659.5 mg, 6.52 mmol). The mixture was stirred at 120° C. overnight. LC-MS showed the reaction was complete. The reaction mixture was diluted with DCM (20 mL), then washed with water and brine, dried over anhydrous Na₂SO₄, filtered and concentrated. The residue was purified by column chromatography on silica gel (DCM:MeOH=20:1, V/V) to afford the desired product (150 mg, yield: 21%). LC/MS(ESI): m/z 323 [M+H]⁺.

### Step 3. (3R)-4-[4-(diethylphosphoryl)-5,7-diiodoimidazo[1,5-b]pyridazin-2-yl]-3-methylmorpholine

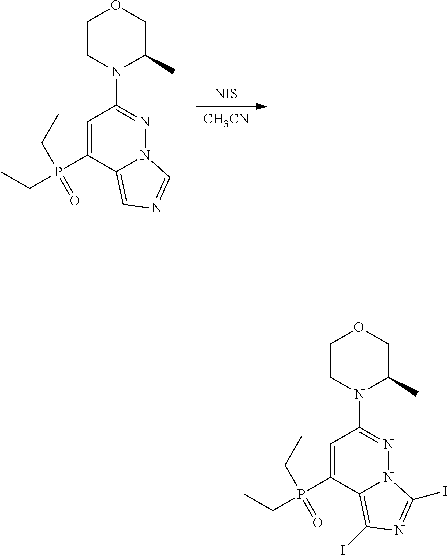

To a solution of (3R)-4-[4-(diethylphosphoryl)imidazo[1,5-b]pyridazin-2-yl]-3-methylmorpholine (150 mg, 0.47 mmol) in MeCN (15 mL) were added NIS (523.5 mg, 2.33 mmol) portion wise. The mixture was stirred at room temperature overnight. LC-MS showed the reaction was complete. The reaction mixture was diluted with EA (20 mL), then washed with water and brine, dried over anhydrous Na₂SO₄, filtered and concentrated. The residue was purified by column chromatography on silica gel (DCM:MeOH=20:1, V/V) to afford the desired product (180 mg, yield: 67%). LC/MS(ESI): m/z 575 [M+H]⁺.

### Step 4. (3R)-4-[4-(diethylphosphoryl)-5-iodo-7-[1-(oxan-2-yl)-1H-pyrazol-5-yl]imidazo[1,5-b]pyridazin-2-yl]-3-methylmorpholine

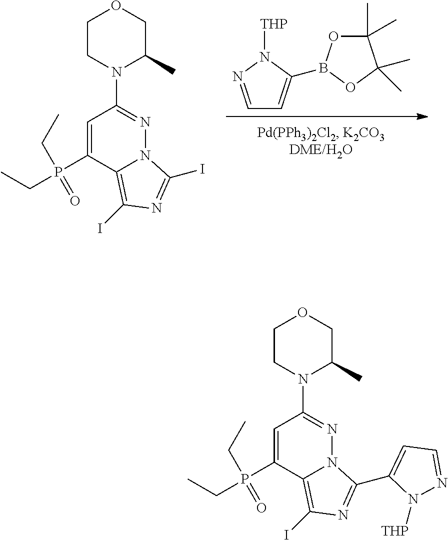

To a solution of (3R)-4-[4-(diethylphosphoryl)-5,7-diiodoimidazo[1,5-b]pyridazin-2-yl]-3-methylmorpholine (180 mg, 0.31 mmol) and 1-(oxan-2-yl)-5-(tetramethyl-1,3,2-dioxaborolan-2-yl)-1H-pyrazole (130.8 mg, 0.47 mmol) in co-solvent of DME (10 mL) and H₂O (2 mL) were added K₂CO₃ (130.0 mg, 0.94 mmol) and Pd(PPh₃)₂Cl₂ (22.0 mg, 0.03 mmol). The mixture was stirred at 80° C. overnight under nitrogen atmosphere. The reaction mixture was diluted with EA (20 mL), then washed with water and brine, dried over anhydrous Na₂SO₄, filtered and concentrated. The residue was purified by column chromatography on silica gel (DCM:MeOH=20:1, V/V) to afford the desired product (120 mg, yield: 64%). LC/MS(ESI): m/z 599 [M+H]⁺.

### Step 5. (3R)-4-[4-(diethylphosphoryl)-7-(1H-pyrazol-5-yl)imidazo[1,5-b]pyridazin-2-yl]-3-methylmorpholine

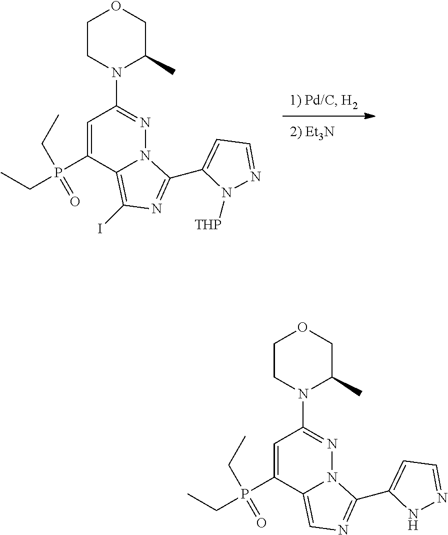

To a solution of (3R)-4-[4-(diethylphosphoryl)-5-iodo-7-[1-(oxan-2-yl)-1H-pyrazol-5-yl]imidazo[1,5-b]pyridazin-2-yl]-3-methylmorpholine (120 mg, 0.20 mmol) in MeOH (6 mL) was added Pd/C (10%, 20 mg). The mixture was stirred at room temperature overnight under H₂ atmosphere. A drop of Et₃N was added to the above solution, then the resulting mixture was continued to stir at room temperature for an additional 2 h under H₂ atmosphere. LC-MS showed the reaction was complete. The reaction mixture was filtered and concentrated. The residue was purified by Prep-HPLC (C18, 10-95%, MeOH in H₂O with 0.1% HCOOH) to give the desired product (20 mg, yield: 25%). LC/MS (ESI): m/z 389 [M+H]⁺. ¹H NMR (400 MHz, DMSO) δ 7.83 (s, 1H), 7.74 (s, 1H), 7.11 (d, J=1.6 Hz, 1H), 7.04 (d, J=13.9 Hz, 1H), 4.35 (d, J=6.3 Hz, 1H), 4.01 (dd, J=11.2, 2.9 Hz, 1H), 3.88 (d, J=12.6 Hz, 1H), 3.75 (dt, J=11.6, 6.9 Hz, 2H), 3.56 (dt, J=13.2, 9.9 Hz, 1H), 3.28 (d, J=12.8 Hz, 1H), 2.34-1.95 (m, 4H), 1.24 (d, J=6.7 Hz, 3H), 1.03 (dt, J=17.3, 7.6 Hz, 6H).

### Example 40

### Synthesis of (R)-2-methyl-2-(2-(3-methylmorpholino)-7-(1H-pyrazol-5-yl)imidazo[1,5-b]pyridazin-4-yl)propanenitrile

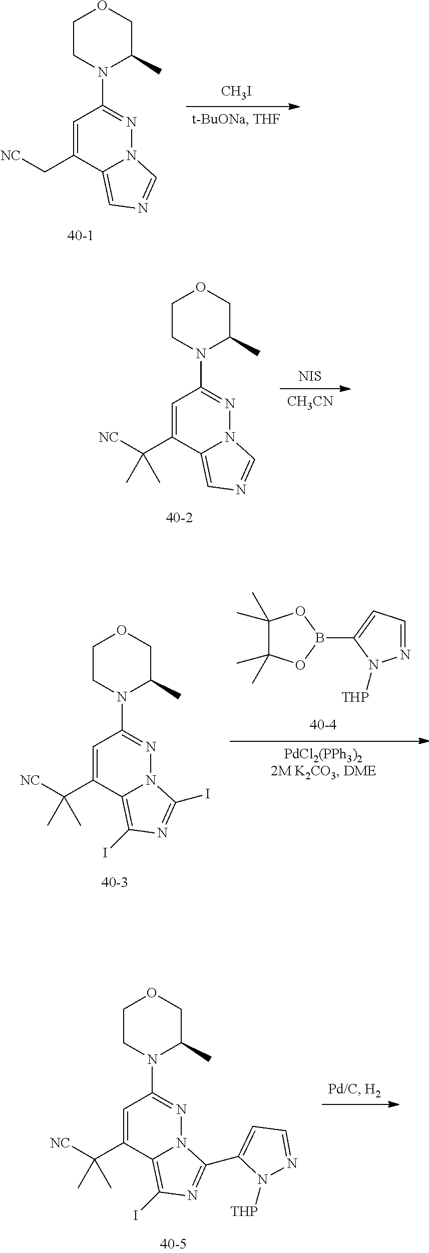

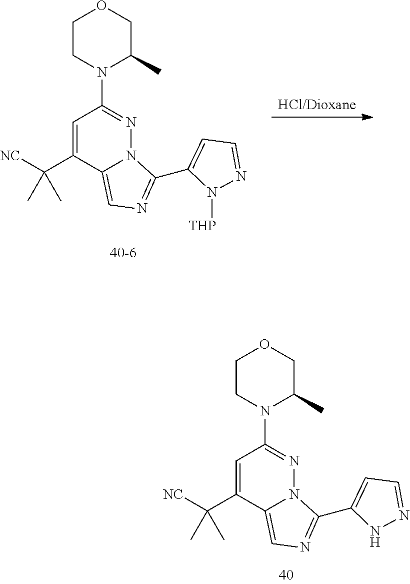

### Step 1. (R)-2-methyl-2-(2-(3-methylmorpholino)imidazo[1,5-b]pyridazin-4-yl) propanenitrile

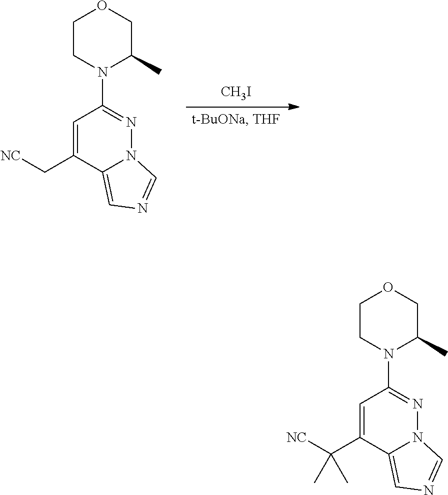

To a solution of 2-{2-[(3R)-3-methylmorpholin-4-yl]imidazo[1,5-b]pyridazin-4-yl}acetonitrile (100 mg, 0.39 mmol) and t-BuONa (96 mg, 0.77 mmol) in anhydrous THF (5 mL) at 0° C. was added a solution of CH₃I (110 mg, 0.77 mmol) in anhydrous THF (1 mL) drop wise. After the addition, the resulting mixture was stirred at 0° C. for 1 h. LC-MS showed the reaction was complete. The reaction mixture was diluted with EA (40 mL), then washed with water and brine, dried over anhydrous Na₂SO₄, filtered and concentrated. The residue was purified by column chromatography on silica gel (DCM:MeOH=20:1, V/V) to afford the desired product (110 mg, yield: 76%). LC/MS (ESI): m/z 286 [M+H]⁺.

### Step 2. (R)-2-(5,7-diiodo-2-(3-methylmorpholino)imidazo[1,5-b]pyridazin-4-yl)-2-methylpropanenitrile

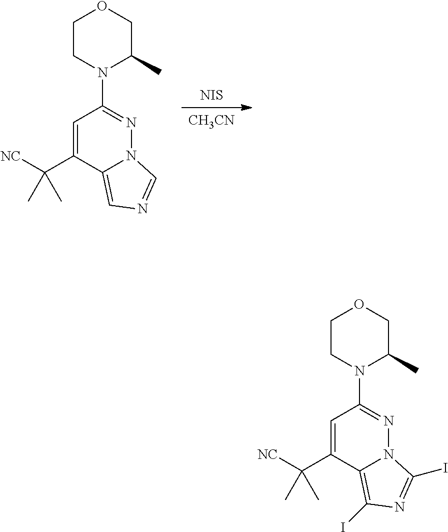

A mixture of 2-methyl-2-{2-[(3R)-3-methylmorpholin-4-yl]imidazo[1,5-b]pyridazin-4-yl}propanenitrile (85 mg, 0.29 mmol) and NIS (268 mg, 1.19 mmol) in MeCN (4 mL) was stirred at 80° C. for 16 h. LC-MS showed the reaction was complete. The reaction mixture was diluted with DCM (20 mL), then washed with saturated Na₂S₂O₃ aqueous solution and brine, dried over anhydrous Na₂SO₄, filtered and concentrated. The residue was purified by column chromatography on silica gel (PE:EA=1:1, V/V) to afford the desired product (91 mg, yield: 56%). LC/MS (ESI): m/z 538 [M+H]⁺.

### Step 3. 2-(5-iodo-2-((R)-3-methylmorpholino)-7-(1-(tetrahydro-2H-pyran-2-yl)-1H-pyrazol-5-yl)imidazo[1,5-b]pyridazin-4-yl)-2-methylpropanenitrile

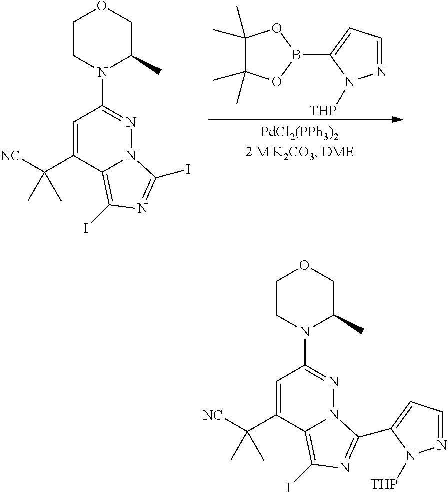

To a solution of 2-{5,7-diiodo-2-[(3R)-3-methylmorpholin-4-yl]imidazo[1,5-b] pyridazin-4-yl}-2-methylpropanenitrile (45 mg, 0.08 mmol) and 1-(oxan-2-yl)-5-(tetramethyl-1,3,2-dioxaborolan-2-yl)-1H-pyrazole (35 mg, 0.12 mmol) in DME (3 mL) were added PdCl₂(PPh₃)₂ (11 mg, 0.02 mmol) and K₂CO₃ (2.0 M in H₂O, 0.12 mL, 0.24 mmol). The mixture was stirred at 100° C. for 16 h under N₂ atmosphere. LC-MS showed the reaction was complete. The reaction mixture was diluted with EA (40 mL), then washed with water and brine, dried over anhydrous Na₂SO₄, filtered and concentrated. The residue was purified by column chromatography on silica gel (PE:EA=1:1, V/V) to afford the desired product (15 mg, yield: 31%). LC/MS (ESI): m/z 562 [M+H]⁺.

### Step 4. 2-methyl-2-(2-((R)-3-methylmorpholino)-7-(1-(tetrahydro-2H-pyran-2-yl)-1H-pyrazol-5-yl)imidazo[1,5-b]pyridazin-4-yl)propanenitrile

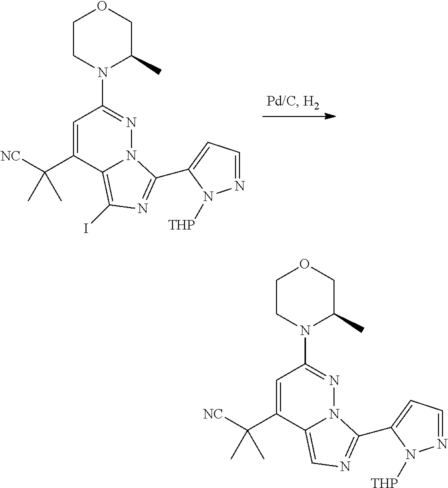

A mixture of 2-{5-iodo-2-[(3R)-3-methylmorpholin-4-yl]-7-[1-(oxan-2-yl)-1H-pyrazol-5-yl]imidazo[1,5-b]pyridazin-4-yl}-2-methylpropanenitrile (85 mg, 0.15 mmol) and Pd/C (10%, 40 mg) in MeOH (3 mL) was stirred at room temperature for 5 h under H₂ atmosphere. LC-MS showed the reaction was complete. The reaction mixture was filtered, then the filtrate was concentrated under reduced pressure. The residue was purified by column chromatography on silica gel (DCM:MeOH=20:1, V/V) to afford the desired product (40 mg, yield: 60%). LC/MS (ESI): m/z 436 [M+H]⁺.

### Step 5. (R)-2-methyl-2-(2-(3-methylmorpholino)-7-(1H-pyrazol-5-yl)imidazo[1,5-b]pyridazin-4-yl)propanenitrile

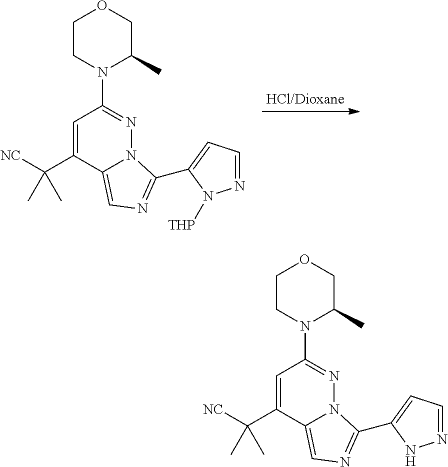

A mixture of 2-methyl-2-{2-[(3R)-3-methylmorpholin-4-yl]-7-[1-(oxan-2-yl)-1H-pyrazol-5-yl]imidazo[1,5-b]pyridazin-4-yl}propanenitrile (40 mg, 0.09 mmol) in HCl solution (4.0 M in dioxane, 3.0 mL) was stirred at room temperature for 1 h. LC-MS showed the reaction was complete. The reaction mixture was concentrated under reduced pressure. The residue was purified by Prep-HPLC (C18, 10-95%, MeOH in H₂O with 0.1% HCOOH) to give the desired product (20 mg, yield: 61%). LC/MS (ESI): m/z 352 [M+H]⁺. ¹H NMR (400 MHz, DMSO) δ 13.21 (s, 1H), 8.14 (s, 1H), 7.77 (s, 1H), 7.72 (d, J=1.2 Hz, 1H), 7.10 (d, J=1.9 Hz, 1H), 6.73 (s, 1H), 4.35 (d, J=6.6 Hz, 1H), 4.01 (dd, J=11.3, 3.1 Hz, 1H), 3.86 (d, J=13.1 Hz, 1H), 3.78 (d, J=11.4 Hz, 1H), 3.71 (dd, J=11.5, 2.7 Hz, 1H), 3.56 (td, J=11.8, 2.9 Hz, 1H), 3.29 (d, J=3.6 Hz, 1H), 1.88 (d, J=1.2 Hz, 6H), 1.25 (d, J=6.7 Hz, 3H).

### Example 41

### Synthesis of (3R)-4-[4-(2-methanesulfonylpropan-2-yl)-7-(1H-pyrazol-5-yl)imidazo[1,5-b]pyridazin-2-yl]-3-methylmorpholine

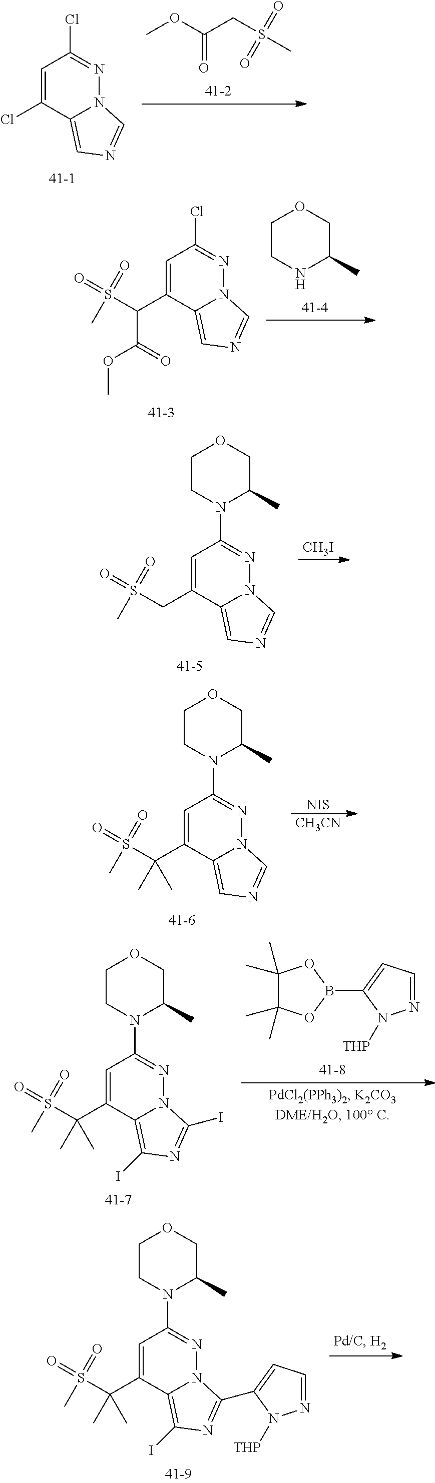

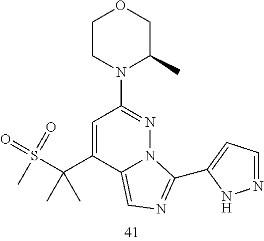 [SMILES: C[C@@H]1COCCN1c1cc(C(C)(C)S(C)(=O)=O)c2cnc(-c3ccn[nH]3)n2n1]

### Step 1. methyl 2-{2-chloroimidazo[1,5-b]pyridazin-4-yl}-2-methanesulfonylacetate

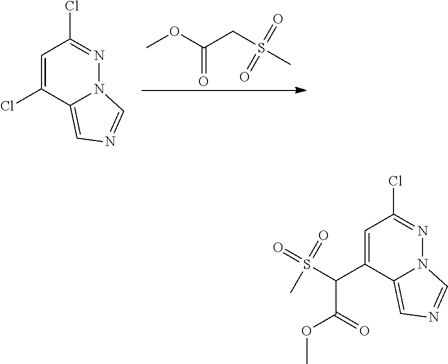

To a solution of 2,4-dichloroimidazo[1,5-b]pyridazine (1 g, 5.32 mmol) in CH₃CN (20 mL) were added methyl 2-methanesulfonylacetate (1.21 g, 7.98 mmol) and Cs₂CO₃ (3.47 g, 10.64 mmol). The reaction was stirred at 60° C. for 6 h. LC-MS showed the reaction was complete. The reaction mixture was diluted with DCM (20 mL), then washed with water and brine, dried over anhydrous Na₂SO₄, filtered and concentrated. The residue was purified by column chromatography on silica gel (PE:EA=2:1, V/V) to afford the desired product (755 mg, yield: 47%). LC/MS (ESI): m/z 304 [M+H]⁺.

### Step 2. (3R)-4-[4-(methanesulfonylmethyl)imidazo[1,5-b]pyridazin-2-yl]-3-methyl morpholine

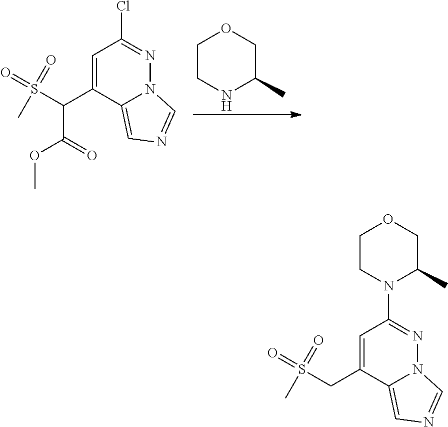

To a solution of methyl 2-{2-chloroimidazo[1,5-b]pyridazin-4-yl}-2-methane sulfonylacetate (755 mg, 2.49 mmol) in sulfolane (10 mL) were added (3R)-3-methyl morpholine (754 mg, 7.46 mmol) and KF (432 mg, 7.46 mmol). The mixture was stirred at 180° C. for 5 h. LC-MS showed the reaction was complete. The reaction mixture was diluted with DCM (20 mL), then washed with water and brine, dried over anhydrous Na₂SO₄, filtered and concentrated. The residue was purified by column chromatography on silica gel (PE:EA=1:1, V/V) to afford the desired product (490 mg, yield: 64%). LC/MS (ESI): m/z 311 [M+H]⁺.

### Step 3. (3R)-4-[4-(2-methanesulfonylpropan-2-yl)imidazo[1,5-b]pyridazin-2-yl]-3-methylmorpholine

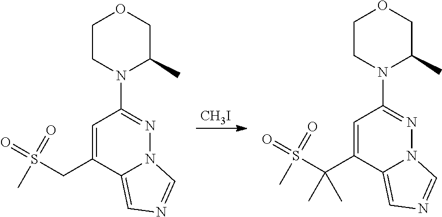

To a solution of (3R)-4-[4-(methanesulfonylmethyl)imidazo[1,5-b]pyridazin-2-yl]-3-methylmorpholine (300 mg, 0.97 mmol) in THF (9 mL) were added iodomethane (0.24 mL, 3.87 mmol) and Sodium tert-butoxide (371.5 mg, 3.87 mmol). LC-MS showed the reaction was complete. The reaction mixture was diluted with DCM (20 mL), then washed with water and brine, dried over anhydrous Na₂SO₄, filtered and concentrated. The residue was purified by column chromatography on silica gel (PE:EA=1:1, V/V) to afford the desired product (210 mg, yield: 64%). LC/MS (ESI): m/z 339 [M+H]⁺.

### Step 4. (3R)-4-[5,7-diiodo-4-(2-methanesulfonylpropan-2-yl)imidazo[1,5-b]pyridazin-2-yl]-3-methylmorpholine

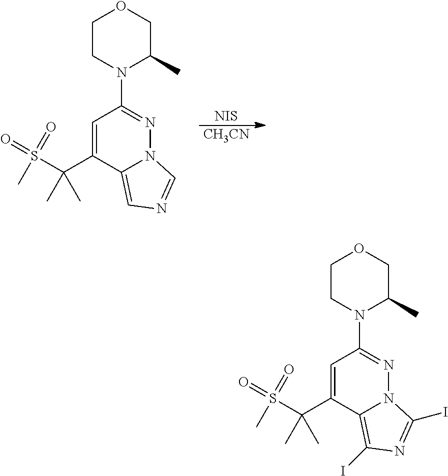

To a solution of (3R)-4-[4-(2-methanesulfonylpropan-2-yl)imidazo[1,5-b]pyridazin-2-yl]-3-methylmorpholine (210 mg, 0.62 mmol) in CH₃CN (20 mL) was added NIS (107 mg, 0.62 mmol) portion wise. The reaction was stirred at 80° C. overnight. LC-MS showed the reaction was complete. The reaction mixture was diluted with DCM (20 mL), then washed with saturated Na₂S₂O₃ aqueous solution and brine, dried over anhydrous Na₂SO₄, filtered and concentrated. The residue was purified by column chromatography on silica gel (PE:EA=1:1, V/V) to afford the desired product (50 mg, yield: 14%). LC/MS (ESI): m/z 591 [M+H]⁺.

### Step 5. (3R)-4-[5-iodo-4-(2-methanesulfonylpropan-2-yl)-7-[1-(oxan-2-yl)-1H-pyrazol-5-yl]imidazo[1,5-b]pyridazin-2-yl]-3-methylmorpholine

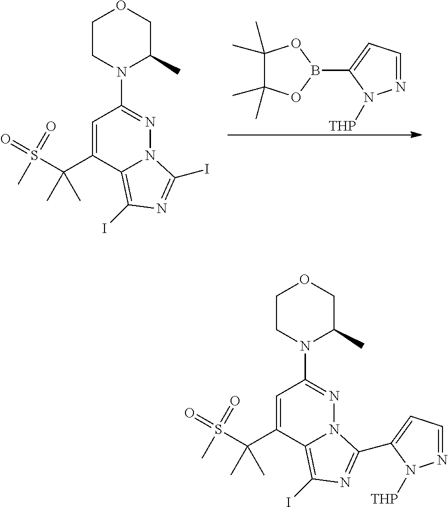

To a solution of (3R)-4-[5,7-diiodo-4-(2-methanesulfonylpropan-2-yl)imidazo [1,5-b] pyridazin-2-yl]-3-methylmorpholine (80 mg, 0.14 mmol) in DME (5 mL) were added 1-(oxan-2-yl)-5-(tetramethyl-1,3,2-dioxaborolan-2-yl)-1H-pyrazole (76 mg, 0.27 mmol), Pd(PPh₃)₂Cl₂ (19 mg, 0.03 mmol) and K₂CO₃ (56 mg, 0.41 mmol). The mixture was stirred at 100° C. overnight under nitrogen atmosphere. LC-MS showed the reaction was complete. The reaction mixture was diluted with DCM (20 mL), then washed with water and brine, dried over anhydrous Na₂SO₄, filtered and concentrated. The residue was purified by column chromatography on silica gel (PE:EA=1:1, V/V) to afford the desired product (42 mg, yield: 51%). LC/MS (ESI): m/z 615 [M+H]⁺.

### Step 6. (3R)-4-[4-(2-methanesulfonylpropan-2-yl)-7-(1H-pyrazol-5-yl)imidazo[1,5-b] pyridazin-2-yl]-3-methylmorpholine

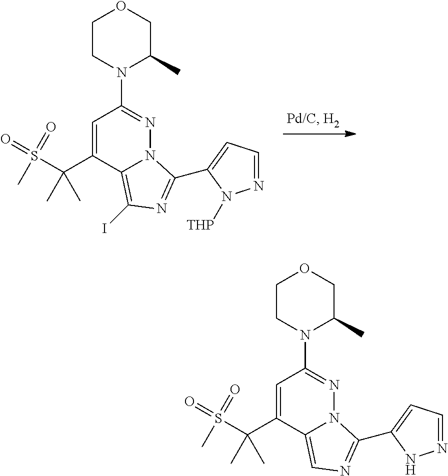

To a solution of (3R)-4-[5-iodo-4-(2-methanesulfonylpropan-2-yl)-7-[1-(oxan-2-yl)-1H-pyrazol-5-yl]imidazo[1,5-b]pyridazin-2-yl]-3-methylmorpholine (42 mg, 0.07 mmol) in MeOH (4 mL) was added Pd/C (10%, 40 mg). The mixture was stirred at room temperature for 12 h under H₂ atmosphere. LC-MS showed the reaction was completed. The reaction mixture was filtered, the filtrate was concentrated under reduced pressure. The residue was purified by Prep-HPLC (C18, 10-95%, MeOH in H₂O with 0.1% HCOOH) to afford the desired product (2.5 mg, yield: 9%). LC/MS (ESI): m/z 405 [M+H]⁺. ¹H NMR (400 MHz, DMSO) δ 13.29 (d, J=159.7 Hz, 1H), 7.68 (d, J=29.9 Hz, 2H), 7.09 (s, 1H), 6.82 (s, 1H), 4.37 (s, 1H), 4.02 (d, J=8.8 Hz, 1H), 3.91-3.59 (m, 3H), 3.57 (dt, J=11.7, 5.9 Hz, 1H), 3.30-3.17 (m, 1H), 2.94 (s, 3H), 1.92 (t, J=7.6 Hz, 6H), 1.23 (d, J=6.7 Hz, 3H).

### Example 42

### Synthesis of (R)-3-methyl-4-(7-(3-methyl-1H-pyrazol-5-yl)-4-(2-(methyl sulfonyl)propan-2-yl)imidazo[1,5-b]pyridazin-2-yl)morpholine

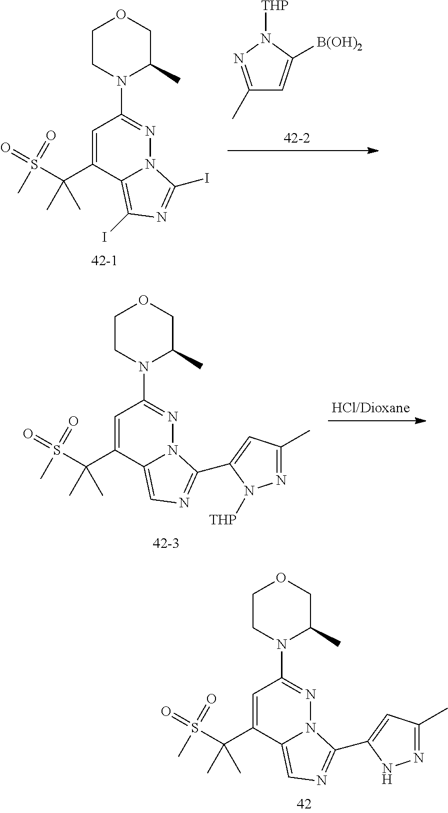

### Step 1. (3R)-3-methyl-4-(7-(3-methyl-1-(tetrahydro-2H-pyran-2-yl)-1H-pyrazol-5-yl)-4-(2-(methylsulfonyl)propan-2-yl)imidazo[1,5-b]pyridazin-2-yl)morpholine

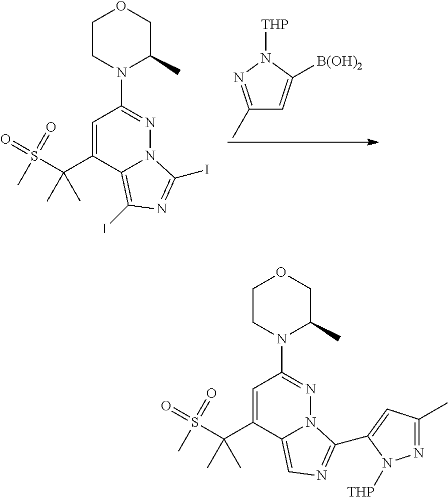

To a solution of (R)-4-(5,7-diiodo-4-(2-(methylsulfonyl)propan-2-yl)imidazo[1,5-b] pyridazin-2-yl)-3-methylmorpholine (100 mg, 0.17 mmol) in DME (20 mL) was added (3-methyl-1-(tetrahydro-2H-pyran-2-yl)-1H-pyrazol-5-yl)boronic acid (71 mg, 0.34 mmol), K₂CO₃ (2M in H₂O, 0.25 mL, 0.51 mmol) and Bis(triphenyl-phosphine)palladium(II) chloride (13 mg, 0.02 mmol). The reaction was stirred at 100° C. overnight under nitrogen atmosphere. LC-MS showed the reaction was complete. The reaction mixture was diluted with DCM (20 mL), then washed with water and brine, dried over anhydrous Na₂SO₄, filtered and concentrated. The residue was purified by column chromatography on silica gel (DCM:MeOH=30:1, V/V) to afford the desired product (30 mg, yield: 35%). LC/MS (ESI): m/z 503 [M+H]⁺.

### Step 2. (R)-3-methyl-4-(7-(3-methyl-1H-pyrazol-5-yl)-4-(2-(methylsulfonyl)propan-2-yl)imidazo[1,5-b]pyridazin-2-yl)morpholine

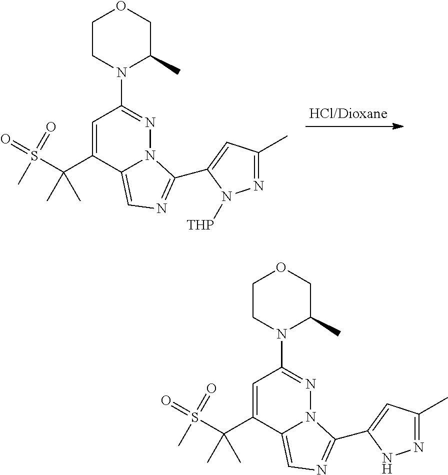

A solution of (3R)-3-methyl-4-(7-(3-methyl-1-(tetrahydro-2H-pyran-2-yl)-1H-pyrazol-5-yl)-4-(2-(methylsulfonyl)propan-2-yl)imidazo[1,5-b]pyridazin-2-yl)morpholine (30 mg, 0.06 mmol) in HCl solution (4M in dioxane, 2 mL) was stirred at room temperature for 1 h. LC-MS showed the reaction was complete. The reaction mixture was concentrated in vacuo. The residue was purified by Pre-HPLC (C₁₈, 10-95%, MeOH in H₂O with 0.1% HCOOH) to afford the desired product (7 mg, yield: 28%). LC/MS (ESI) m/z: 419 [M+H]⁺. ¹H NMR (400 MHz, DMSO) δ 7.68 (s, 1H), 6.84 (s, 1H), 6.79 (s, 1H), 4.34 (q, J=6.8 Hz, 1H), 4.02 (dd, J=11.4, 3.1 Hz, 1H), 3.85 (d, J=13.1 Hz, 1H), 3.81-3.72 (m, 2H), 3.58 (dd, J=11.8, 8.9 Hz, 1H), 3.24 (d, J=3.8 Hz, 1H), 2.94 (s, 3H), 2.28 (s, 3H), 1.92 (d, J=1.1 Hz, 6H), 1.23 (d, J=6.7 Hz, 3H).

### Example 44

### Synthesis of (R)-1-(2-(3-methylmorpholino)-7-(1H-pyrazol-5-yl)imidazo[1,5-b]pyridazin-4-yl)cyclopropane-1-carbonitrile

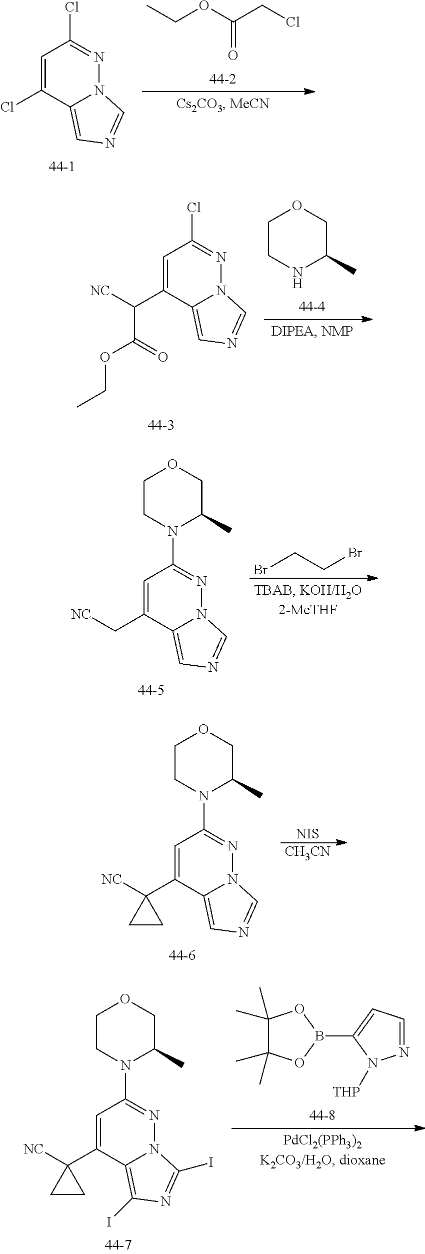

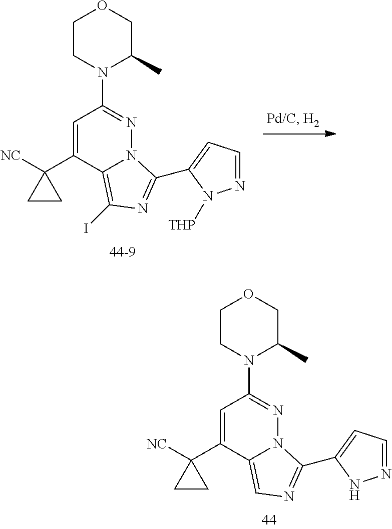

### Step 1. ethyl 2-(2-chloroimidazo[1,5-b]pyridazin-4-yl)-2-cyanoacetate

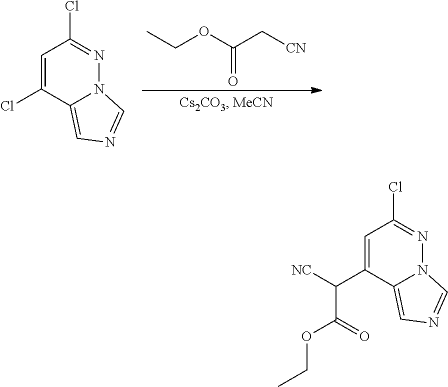

A mixture of 2,4-dichloroimidazo[1,5-b]pyridazine (500 mg, 2.65 mmol), ethyl 2-cyanoacetate (453 mg, 40 mmol) and Cs₂CO₃ (1.74 g, 5.34 mmol) in MeCN (10 mL) was stirred at 60° C. for 3 h. LC-MS showed the reaction was complete. The reaction mixture was diluted with DCM (20 mL), then washed with water and brine, dried over anhydrous Na₂SO₄, filtered and concentrated. The residue was purified by column chromatography on silica gel (DCM:MeOH=5:1, V/V) to afford the desired product (600 mg, yield: 85%). LC/MS (ESI): m/z 265 [M+H]⁺.

### Step 2. (R)-2-(2-(3-methylmorpholino)imidazo[1,5-b]pyridazin-4-yl)acetonitrile

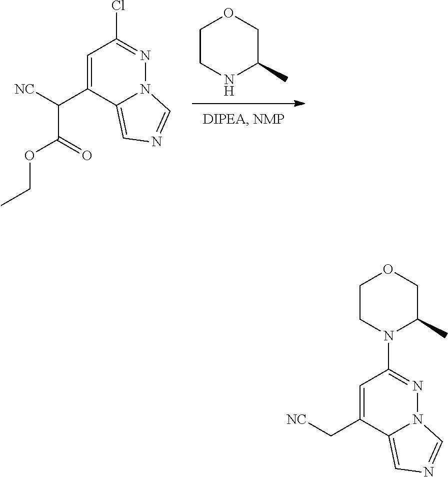

A mixture of ethyl 2-{2-chloroimidazo[1,5-b]pyridazin-4-yl}-2-cyanoacetate (200 mg, 0.75 mmol), (3R)-3-methylmorpholine (306 mg, 3.02 mmol) and DIPEA (390 mg, 3.02 mmol) in NMP (5 mL) was stirred at 200° C. for 5 h. LC-MS showed the reaction was complete. The reaction mixture was diluted with DCM (20 mL), then washed with water and brine, dried over anhydrous Na₂SO₄, filtered and concentrated. The residue was purified by column chromatography on silica gel (DCM:MeOH=15:1, V/V) to afford the desired product (50 mg, yield: 25%). LC/MS (ESI): m/z 258 [M+H]⁺.

### Step 3. (R)-1-(2-(3-methylmorpholino)imidazo[1,5-b]pyridazin-4-yl)cyclopropane-1-carbonitrile

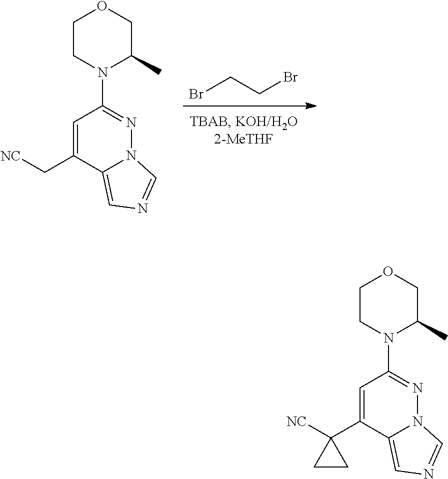

A mixture of 2-{2-[(3R)-3-methylmorpholin-4-yl]imidazo[1,5-b]pyridazin-4-yl} acetonitrile (200 mg, 0.77 mmol), 1,2-dibromoethane (580 mg, 3.08 mmol), TBAB (50 mg, 0.15 mmol) and KOH (10.0 M in H₂O, 1.5 mL, 15 mmol) in 2-Methyltetrahydrofuran (20 mL) was stirred at 80° C. for 4 h. LC-MS showed the reaction was complete. The reaction mixture was diluted with EA (40 mL), then washed with water and brine, dried over anhydrous Na₂SO₄, filtered and concentrated. The residue was purified by column chromatography on silica gel (PE:EA=1:1, V/V) to afford the desired product (180 mg, yield: 81%). LC/MS (ESI): m/z 284 [M+H]⁺.

### Step 4. (R)-1-(5,7-diiodo-2-(3-methylmorpholino)imidazo[1,5-b]pyridazin-4-yl)cyclopropane-1-carbonitrile

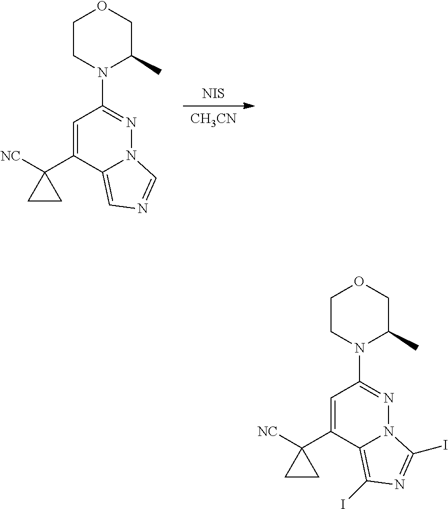

A mixture of 1-{2-[(3R)-3-methylmorpholin-4-yl]imidazo[1,5-b]pyridazin-4-yl} cyclopropane-1-carbonitrile (200 mg, 0.70 mmol) and NIS (640 mg, 2.84 mmol) in MeCN (8 mL) was stirred at room temperature for 4 h. LC-MS showed the reaction was complete. The reaction mixture was diluted with EA (40 mL), then washed with water and brine, dried over anhydrous Na₂SO₄, filtered and concentrated. The residue was purified by column chromatography on silica gel (PE:EA=1:1, V/V) to afford the desired product (200 mg, yield: 52%). LC/MS (ESI): m/z 536 [M+H]⁺.

### Step 5. 1-(5-iodo-2-((R)-3-methylmorpholino)-7-(1-(tetrahydro-2H-pyran-2-yl)-1H-pyrazol-5-yl)imidazo[1,5-b]pyridazin-4-yl)cyclopropane-1-carbonitrile

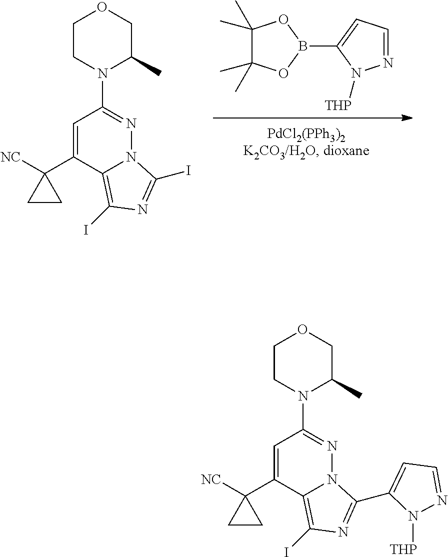

To a solution of 1-{5,7-diiodo-2-[(3R)-3-methylmorpholin-4-yl]imidazo[1,5-b]pyridazin-4-yl}cyclopropane-1-carbonitrile (100 mg, 0.18 mmol) and 1-(oxan-2-yl)-5-(tetramethyl-1,3,2-dioxaborolan-2-yl)-1H-pyrazole (104 mg, 0.37 mmol) in DME (3 mL) was added PdCl₂(PPh₃)₂ (26 mg, 0.18 mmol) and K₂CO₃ (2.0 M in H₂O, 0.28 mL, 0.56 mmol). The mixture was stirred at 100° C. for 16 h under N₂ atmosphere. LC-MS showed the reaction was complete. The reaction mixture was diluted with EA (40 mL), then washed with water and brine, dried over anhydrous Na₂SO₄, filtered and concentrated. The residue was purified by column chromatography on silica gel (DCM:MeOH=1:1, V/V) to afford the desired product (50 mg, yield: 47%). LC/MS (ESI): m/z 560 [M+H]⁺.

### Step 6. (R)-1-(2-(3-methylmorpholino)-7-(1H-pyrazol-5-yl)imidazo[1,5-b]pyridazin-4-yl)cyclopropane-1-carbonitrile

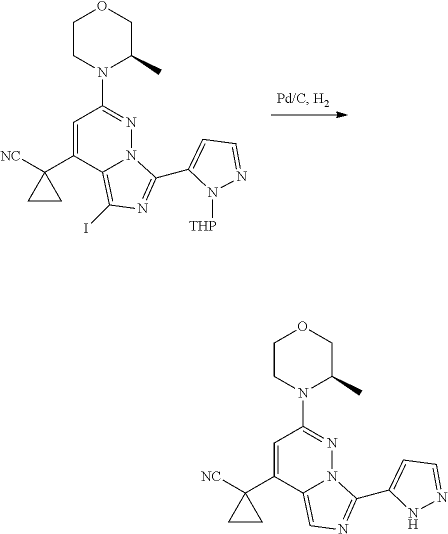

A mixture of 1-{5-iodo-2-[(3R)-3-methylmorpholin-4-yl]-7-[1-(oxan-2-yl)-1H-pyrazol-5-yl]imidazo[1,5-b]pyridazin-4-yl}cyclopropane-1-carbonitrile (24 mg, 0.04 mmol) and Pd/C (10%, 10 mg) in MeOH (3 mL) was stirred at room temperature for 16 h under H₂ atmosphere. LC-MS showed the reaction was complete. The reaction mixture was filtered, then the filtrate was concentrated under reduced pressure. The residue was purified by Prep-HPLC (C18, 10-95%, MeOH in H₂O with 0.1% HCOOH) to afford the desired product (8 mg, yield: 53%). LC/MS (ESI): m/z 350 [M+H]⁺. ¹H NMR (400 MHz, DMSO) δ 13.54 (s, 1H), 8.33 (s, 1H), 7.71 (s, 2H), 7.10 (d, J=1.9 Hz, 1H), 6.79 (s, 1H), 4.37 (d, J=6.4 Hz, 1H), 3.99 (dd, J=11.3, 3.2 Hz, 1H), 3.88 (d, J=12.9 Hz, 1H), 3.77 (d, J=11.3 Hz, 1H), 3.69 (dd, J=11.4, 2.7 Hz, 1H), 3.55 (dd, J=11.9, 2.8 Hz, 1H), 3.26-3.22 (m, 1H), 1.88-1.78 (m, 3H), 1.74 (dd, J=8.4, 4.5 Hz, 1H), 1.23 (d, J=6.7 Hz, 3H).

### Example 45

### Synthesis of (3R)-4-[4-(dimethyl-1H-1,2,3-triazol-5-yl)-5-methyl-7-(1H-pyrazol-5-yl)imidazo[1,5-b]pyridazin-2-yl]-3-methylmorpholine

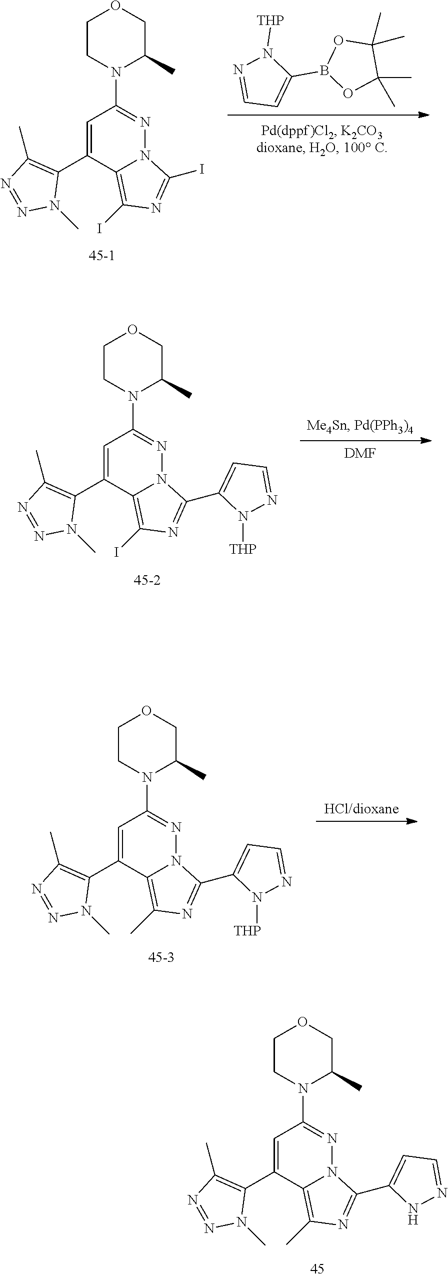

### Step 1. (3R)-4-[4-(dimethyl-1H-1,2,3-triazol-5-yl)-5-iodo-7-[1-(oxan-2-yl)-1H-pyrazol-5-yl]imidazo[1,5-b]pyridazin-2-yl]-3-methylmorpholine

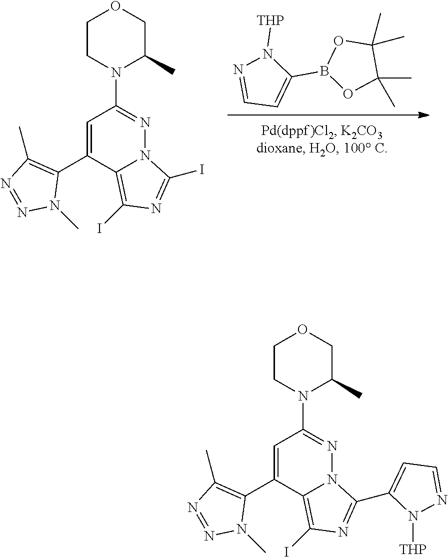

To a solution of (3R)-4-[4-(dimethyl-1H-1,2,3-triazol-5-yl)-5,7-diiodoimidazo[1,5-b] pyridazin-2-yl]-3-methylmorpholine (330 mg, 0.58 mmol) and 1-(oxan-2-yl)-5-(tetra methyl-1,3,2-dioxaborolan-2-yl)-1H-pyrazole (487 mg, 1.75 mmol) in co-solvent of dioxane (10 mL) and H₂O (1 mL) were added Pd(dppf)Cl₂ (43 mg, 0.06 mmol) and Cs₂CO₃ (571 mg, 1.75 mmol). The mixture was stirred at 100° C. overnight under nitrogen atmosphere. LC-MS showed the reaction was complete. The mixture was diluted with EA (50 mL), then washed with water and brine, dried over anhydrous Na₂SO₄, filtered and concentrated. The residue was purified by column chromatography on silica gel (PE:EA=3:1, V/V) to give the desired product (255 mg, yield: 74%). LC/MS ESI (m/z): 590 [M+H]⁺.

### Step 2. (3R)-4-[4-(dimethyl-1H-1,2,3-triazol-5-yl)-5-methyl-7-[1-(oxan-2-yl)-1H-pyrazol-5-yl]imidazo[1,5-b]pyridazin-2-yl]-3-methylmorpholine

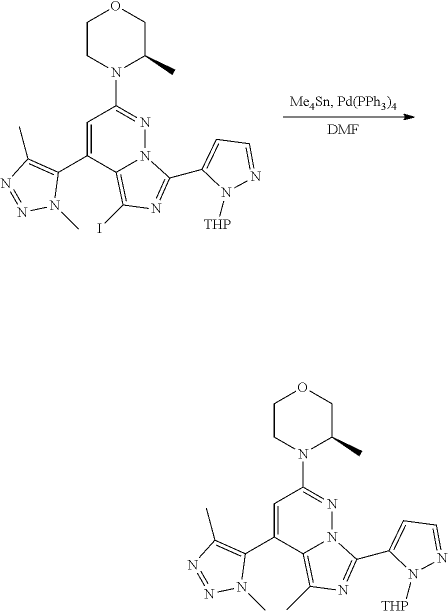

To a solution of (3R)-4-[4-(dimethyl-1H-1,2,3-triazol-5-yl)-5-iodo-7-[1-(oxan-2-yl)-1H-pyrazol-5-yl]imidazo[1,5-b]pyridazin-2-yl]-3-methylmorpholine (120 mg, 0.20 mmol) and tetramethyltin (0.14 mL, 1.02 mmol) in DMF (6 mL) was added Pd(PPh₃)₄ (46 mg, 0.04 mmol). The mixture was stirred at 100° C. overnight under nitrogen atmosphere. LC-MS showed the reaction was complete. The mixture was diluted with EA (50 mL), then washed with water and brine, dried over anhydrous Na₂SO₄, filtered and concentrated. The residue was purified by column chromatography on silica gel (PE:EA=1:1, V/V) to give the desired product (84 mg, yield: 87%). LC/MS ESI (m/z): 478 [M+H]⁺.

### Step 3. (3R)-4-[4-(dimethyl-1H-1,2,3-triazol-5-yl)-5-methyl-7-(1H-pyrazol-5-yl) imidazo[1,5-b]pyridazin-2-yl]-3-methylmorpholine

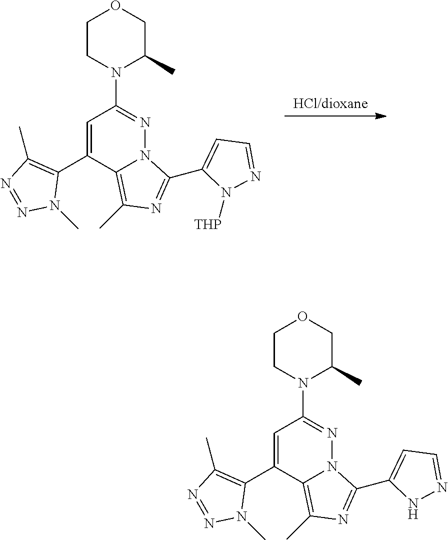

To a solution of (3R)-4-[4-(dimethyl-1H-1,2,3-triazol-5-yl)-5-methyl-7-[1-(oxan-2-yl)-1H-pyrazol-5-yl]imidazo[1,5-b]pyridazin-2-yl]-3-methylmorpholine (84 mg, 0.18 mmol) in DCM (2 mL) was added HCl solution (4M in dioxane, 2 mL). The mixture was stirred at ambient temperature for 1 h. LC-MS showed the reaction was complete. The mixture was concentrated under reduced pressure, the residue was purified by prep-HPLC (C₁₈, 10-95%, MeOH in H₂O with 0.1% HCOOH) to give the desired product (16.5 mg, yield: 24%). LC/MS ESI (m/z): 394 [M+H]⁺. ¹H NMR (400 MHz, DMSO) δ 13.44 (s, 1H), 7.70 (s, 1H), 7.11 (d, J=1.8 Hz, 1H), 6.87 (s, 1H), 4.32 (d, J=5.7 Hz, 1H), 4.00 (dd, J=11.9, 3.8 Hz, 1H), 3.89 (t, J=4.6 Hz, 4H), 3.76 (d, J=11.2 Hz, 1H), 3.70 (d, J=11.5 Hz, 1H), 3.56 (td, J=11.8, 2.8 Hz, 1H), 3.25 (dd, J=12.8, 3.6 Hz, 1H), 2.19 (s, 3H), 1.87 (s, 3H), 1.25 (d, J=6.4 Hz, 3H).

### Example 46

### Synthesis of (3R)-4-[4-(dimethyl-1H-1,2,3-triazol-5-yl)-5-methyl-7-(3-methyl-1H-pyrazol-5-yl)imidazo[1,5-b]pyridazin-2-yl]-3-methylmorpholine

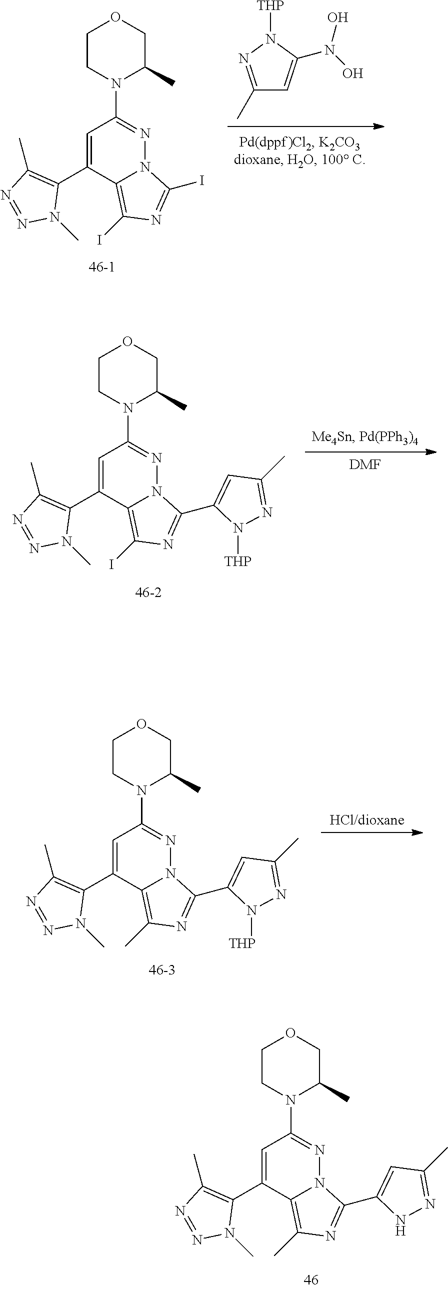

### Step 1. (3R)-4-[4-(dimethyl-1H-1,2,3-triazol-5-yl)-5-iodo-7-[3-methyl-1-(oxan-2-yl)-1H-pyrazol-5-yl]imidazo[1,5-b]pyridazin-2-yl]-3-methylmorpholine

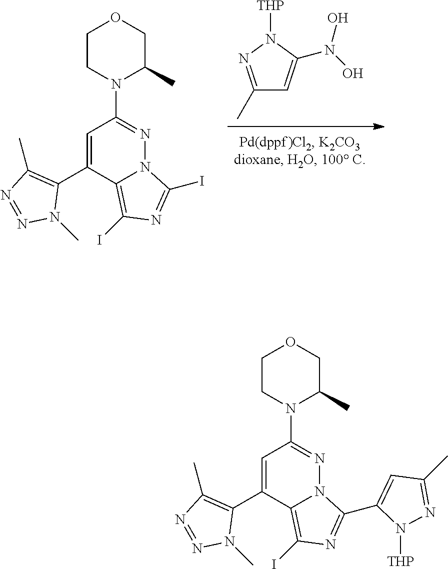

A mixture of (3R)-4-[4-(dimethyl-1H-1,2,3-triazol-5-yl)-5,7-diiodoimidazo[1,5-b] pyridazin-2-yl]-3-methylmorpholine (276 mg, 0.49 mmol), [3-methyl-1-(oxan-2-yl)-1H-pyrazol-5-yl]boronic acid (308 mg, 1.47 mmol), PdCl₂(PPh₃)₂ (69 mg, 0.10 mmol) and Cs₂CO₃ (637 mg, 1.95 mmol) in co-solvent of dioxane (20 mL) and H₂O (2 mL) was stirred at 100° C. overnight under nitrogen atmosphere. LC-MS showed the reaction was complete. The mixture was diluted with EA (50 mL), then washed with water and brine, dried over anhydrous Na₂SO₄, filtered and concentrated. The residue was purified by column chromatography on silica gel (PE:EA=3:1, V/V) to give the desired product (82 mg, yield: 28%). LC/MS ESI (m/z): 604 [M+H]⁺.

### Step 2. (3R)-4-[4-(dimethyl-1H-1,2,3-triazol-5-yl)-5-methyl-7-[3-methyl-1-(oxan-2-yl)-1H-pyrazol-5-yl]imidazo[1,5-b]pyridazin-2-yl]-3-methylmorpholine

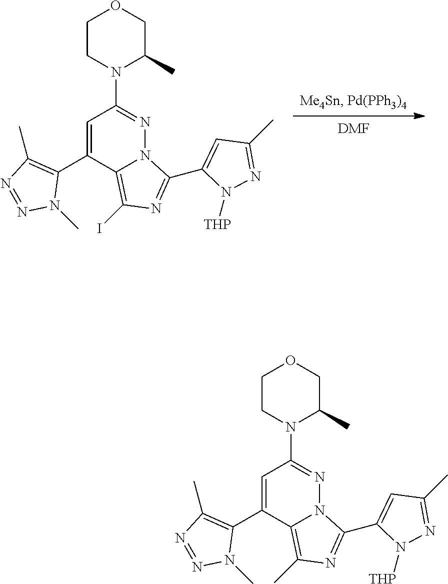

A mixture of (3R)-4-[4-(dimethyl-1H-1,2,3-triazol-5-yl)-5-iodo-7-[3-methyl-1-(oxan-2-yl)-1H-pyrazol-5-yl]imidazo[1,5-b]pyridazin-2-yl]-3-methylmorpholine (25 mg, 0.04 mmol), tetramethyltin (0.03 mL, 0.21 mmol) and Pd(PPh₃)₄ (9.6 mg, 0.01 mmol) in DMF (2 mL) was stirred at 100° C. overnight under nitrogen atmosphere. LC-MS showed the reaction was complete. The mixture was diluted with EA (50 mL), then washed with water and brine, dried over anhydrous Na₂SO₄, filtered and concentrated. The residue was purified by column chromatography on silica gel (PE:EA=1:1, V/V) to give the desired product (18 mg, yield: 88%). LC/MS ESI (m/z): 492 [M+H]⁺.

### Step 3. (3R)-4-[4-(dimethyl-1H-1,2,3-triazol-5-yl)-5-methyl-7-(3-methyl-1H-pyrazol-5-yl)imidazo[1,5-b]pyridazin-2-yl]-3-methylmorpholine

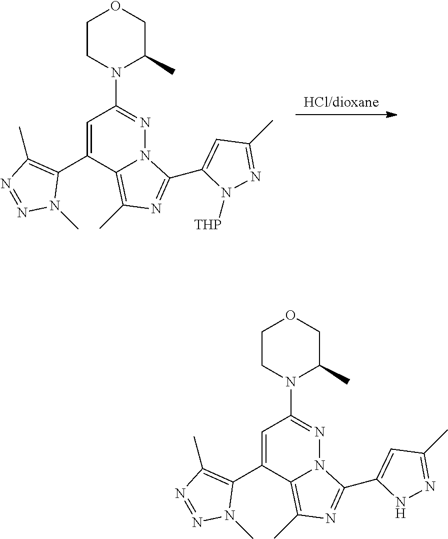

To a solution of (3R)-4-[4-(dimethyl-1H-1,2,3-triazol-5-yl)-5-methyl-7-[3-methyl-1-(oxan-2-yl)-1H-pyrazol-5-yl]imidazo[1,5-b]pyridazin-2-yl]-3-methylmorpholine (18 mg, 0.04 mmol) in DCM (2 mL) was added HCl solution (4M in dioxane, 2 mL). The mixture was stirred at ambient temperature for 1 h. LC-MS showed the reaction was complete. The mixture was concentrated under reduced pressure, the residue was purified by prep-HPLC (C₁₈, 10-95%, MeOH in H₂O with 0.1% HCOOH) to give the desired product (6.7 mg, yield: 45%). LC/MS ESI (m/z): 408 [M+H]⁺. ¹H NMR (400 MHz, DMSO) δ 12.86 (br, 1H), 6.85 (s, 2H), 4.30 (d, J=6.2 Hz, 1H), 4.03-3.97 (m, 1H), 3.93-3.85 (m, 4H), 3.76 (d, J=11.4 Hz, 1H), 3.70 (d, J=11.3 Hz, 1H), 3.56 (dd, J=11.9, 9.3 Hz, 1H), 3.29-3.22 (m, 1H), 2.28 (s, 3H), 2.19 (s, 3H), 1.85 (s, 3H), 1.25 (d, J=6.5 Hz, 3H).

### Example 47

### Synthesis of (R)-3-methyl-4-(5-methyl-4-(1-methyl-1H-pyrazol-5-yl)-7-(3-methyl-1H-pyrazol-5-yl)imidazo[1,5-b]pyridazin-2-yl)morpholine

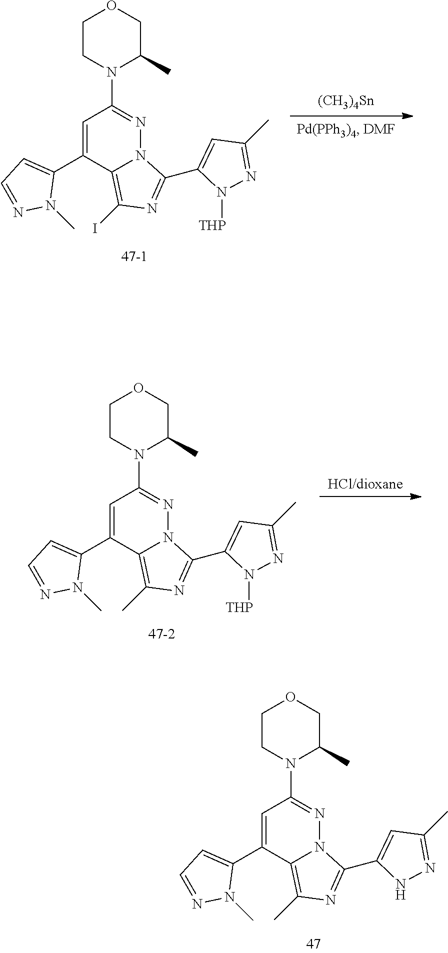

### Step 1. (3R)-3-methyl-4-(5-methyl-7-(3-methyl-1-(tetrahydro-2H-pyran-2-yl)-1H-pyrazol-5-yl)-4-(1-methyl-1H-pyrazol-5-yl)imidazo[1,5-b]pyridazin-2-yl)morpholine

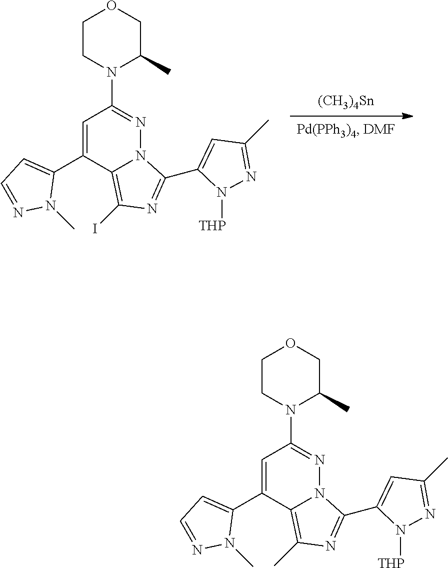

To a solution of (3R)-4-(5-iodo-7-(3-methyl-1-(tetrahydro-2H-pyran-2-yl)-1H-pyrazol-5-yl)-4-(1-methyl-1H-pyrazol-5-yl)imidazo[1,5-b]pyridazin-2-yl)-3-methylmorpholine (100 mg, 0.17 mmol) in DMF (2 mL) was added tetramethylstannane (0.12 mL, 0.85 mmol) and Pd(PPh₃)₄ (39 mg, 0.04 mmol). The reaction was stirred at 100° C. overnight under nitrogen atmosphere. LC-MS showed the reaction was complete. The reaction mixture was diluted with DCM (20 mL), then washed with water and brine, dried over anhydrous Na₂SO₄, filtered and concentrated. The residue was purified by column chromatography on silica gel (DCM:MeOH=30:1, V/V) to afford the desired product (50 mg, yield: 61%). LC/MS (ESI): m/z 477 [M+H]⁺.

### Step 2. (R)-3-methyl-4-(5-methyl-4-(1-methyl-1H-pyrazol-5-yl)-7-(3-methyl-1H-pyrazol-5-yl)imidazo[1,5-b]pyridazin-2-yl)morpholine

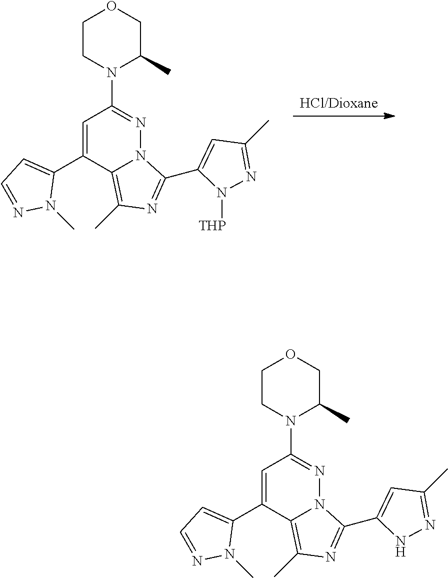

A solution of (3R)-3-methyl-4-(5-methyl-7-(3-methyl-1-(tetrahydro-2H-pyran-2-yl)-1H-pyrazol-5-yl)-4-(1-methyl-1H-pyrazol-5-yl)imidazo[1,5-b]pyridazin-2-yl)morpholine (50 mg, 0.11 mmol) in HCl solution (4M in dioxane, 2 mL) was stirred at room temperature for 1 h. LC-MS showed the reaction was complete. The reaction mixture was concentrated in vacuo. The residue was purified by Pre-HPLC (C₁₈, 10-95%, MeOH in H₂O with 0.1% TFA) to afford the desired product (12 mg, yield: 29%). LC/MS (ESI) m/z: 393 [M+H]⁺. ¹H NMR (400 MHz, DMSO) δ 7.66 (d, J=1.9 Hz, 1H), 7.22 (s, 1H), 7.04 (s, 1H), 6.61 (d, J=1.9 Hz, 1H), 4.44 (d, J=6.5 Hz, 1H), 4.05-3.99 (m, 2H), 3.80 (s, 3H), 3.78 (s, 1H), 3.71 (dd, J=11.7, 2.8 Hz, 1H), 3.56 (dd, J=12.0, 9.2 Hz, 1H), 3.39-3.30 (m, 1H), 2.40 (s, 3H), 2.02 (s, 3H), 1.30 (d, J=6.7 Hz, 3H).

### Example 48

### Synthesis of (R)-4-(7-(1,4-dimethyl-1H-1,2,3-triazol-5-yl)-3-(3-methyl-1H-pyrazol-5-yl)isothiazolo[4,5-b]pyridin-5-yl)-3-methylmorpholine

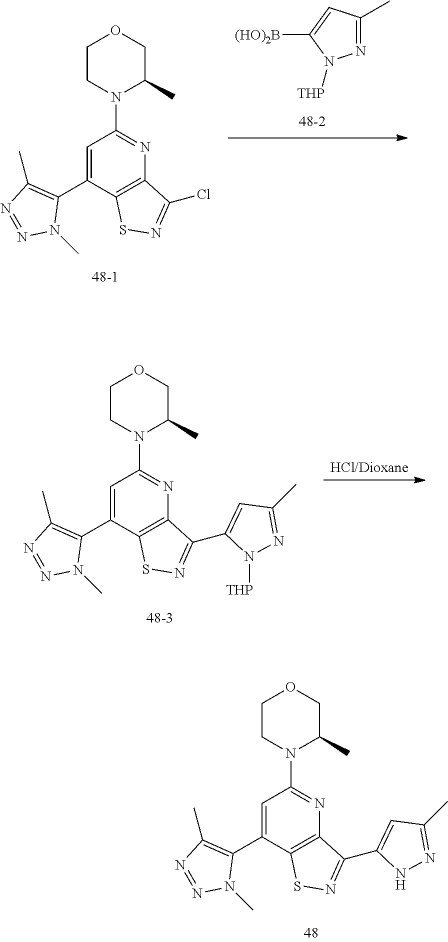

### Step 1. (3R)-4-(7-(1,4-dimethyl-1H-1,2,3-triazol-5-yl)-3-(3-methyl-1-(tetrahydro-2H-pyran-2-yl)-1H-pyrazol-5-yl)isothiazolo[4,5-b]pyridin-5-yl)-3-methylmorpholine

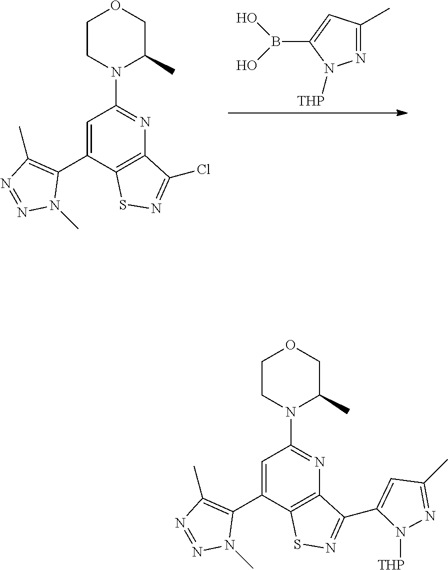

To a mixture of (3R)-4-[3-chloro-7-(dimethyl-1H-1,2,3-triazol-5-yl)-[1,2]thiazolo [4,5-b]pyridin-5-yl]-3-methylmorpholine (15 mg, 0.04 mmol) and [3-methyl-1-(oxan-2-yl)-1H-pyrazol-5-yl]boronic acid (18 mg, 0.08 mmol) in dioxane (3 mL) were added K₂CO₃ (2M in H₂O, 0.06 mL, 0.12 mmol) and Pd(PPh₃)₄ (10 mg, 0.01 mmol). The mixture was stirred at 100° C. for 16 h under N₂ atmosphere. LC-MS showed the reaction was complete. The reaction mixture was diluted with DCM (20 mL), then washed with water and brine, dried over anhydrous Na₂SO₄, filtered and concentrated. The residue was purified by column chromatography on silica gel (PE:EA=1:1, V/V) to afford the desired product (10 mg, yield: 49%). LC/MS (ESI): m/z 495 [M+H]⁺.

### Step 2. (R)-4-(7-(1,4-dimethyl-1H-1,2,3-triazol-5-yl)-3-(3-methyl-1H-pyrazol-5-yl) isothiazolo[4,5-b]pyridin-5-yl)-3-methylmorpholine

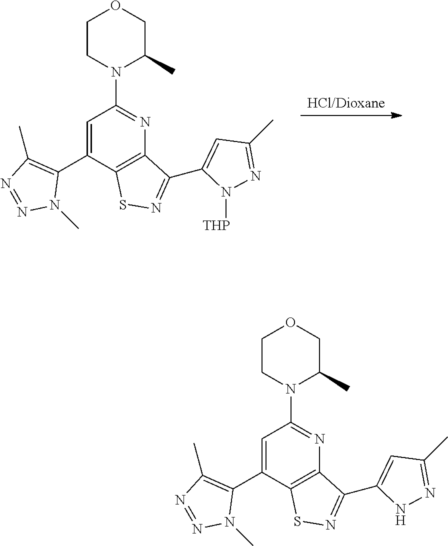

To a mixture of (3R)-4-(7-(1,4-dimethyl-1H-1,2,3-triazol-5-yl)-3-(3-methyl-1-(tetrahydro-2H-pyran-2-yl)-1H-pyrazol-5-yl)isothiazolo[4,5-b]pyridin-5-yl)-3-methyl morpholine (10 mg, 0.02 mmol) in DCM (2 mL) was added HCl solution (4M in dioxane, 1 mL). The mixture was stirred at room temperature for 1 h. LC-MS showed the reaction was complete. The reaction mixture was concentrated under reduced pressure. The residue was purified by Prep-HPLC (C18, 10-95%, MeOH in H₂O with 0.1% HCOOH) to give the desired product (3.5 mg, yield: 42%). LC/MS (ESI): m/z 411 [M+H]⁺. ¹H NMR (400 MHz, DMSO) δ 13.27 (s, 1H), 7.45 (s, 1H), 7.16 (s, 1H), 4.54 (q, J=7.0 Hz, 1H), 4.22-4.16 (m, 1H), 4.05 (dd, J=11.3, 3.0 Hz, 1H), 3.99 (s, 3H), 3.82 (d, J=11.3 Hz, 1H), 3.73 (dd, J=11.4, 2.8 Hz, 1H), 3.58 (dd, J=11.7, 9.1 Hz, 1H), 3.28 (d, J=3.6 Hz, 1H), 2.33 (s, 3H), 2.25 (s, 3H), 1.26 (d, J=6.6 Hz, 3H).

### Example 49

### Synthesis of (R)-3-methyl-4-(7-(1-(methylsulfonyl)cyclopropyl)-3-(1H-pyrazol-5-yl)isothiazolo[4,5-b]pyridin-5-yl)morpholine

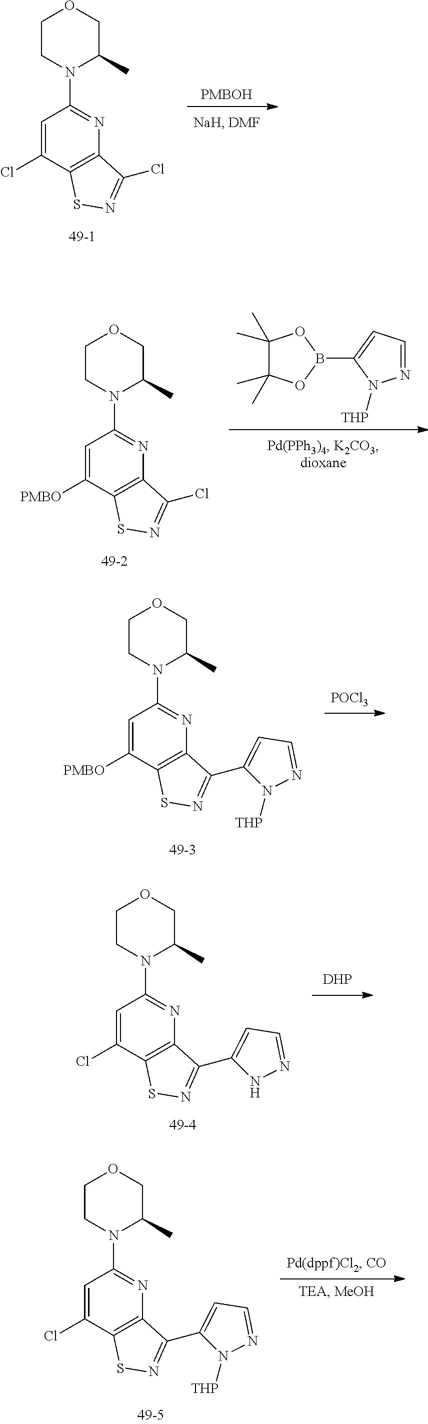

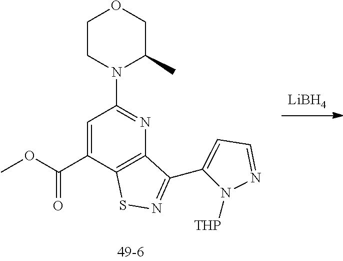

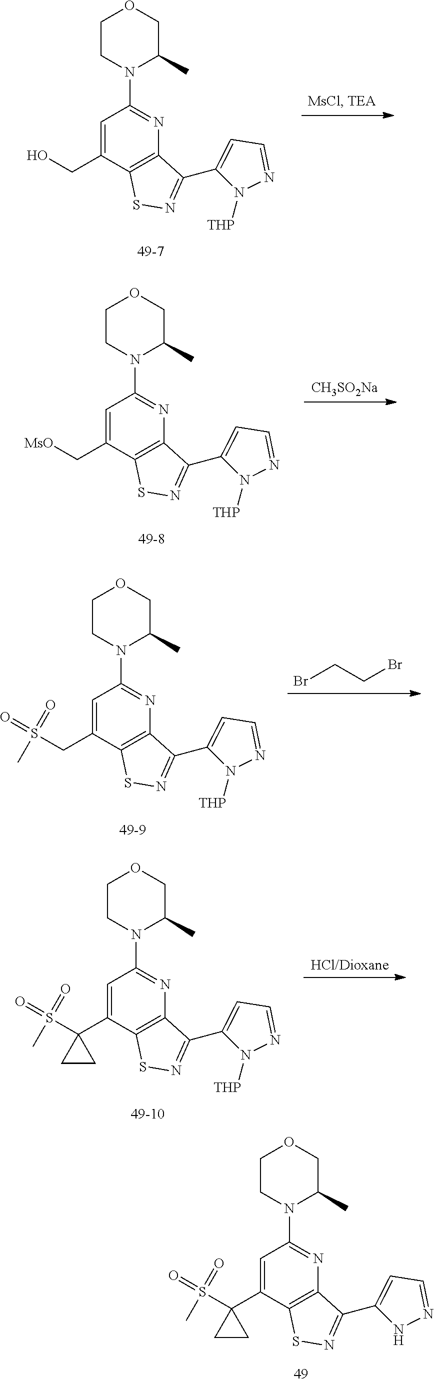

### Step 1. (R)-4-(3-chloro-7-((4-methoxybenzyl)oxy)isothiazolo[4,5-b]pyridin-5-yl)-3-methylmorpholine

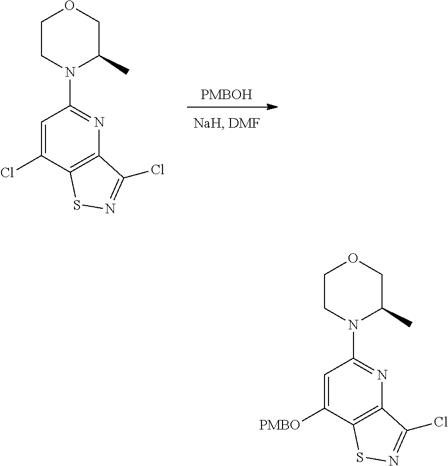

To a solution of 4-Methoxybenzyl alcohol (250 mg, 1.81 mmol) in DMF (10 mL) at 0° C. was added NaH (60% dispersion in mineral oil, 99 mg, 2.47 mmol) portion wise. The mixture was stirred at 0° C. for 15 min, then (3R)-4-{3,7-dichloro-[1,2]thiazolo [4,5-b]pyridin-5-yl}-3-methylmorpholine (500 mg, 1.64 mmol) was added portion wise. The resulting mixture was stirred at 0° C. for 1 h. LC-MS showed the reaction was complete. The reaction mixture was quenched with saturated NH₄Cl aqueous solution, then extracted with EA (50 mL×3). The combined organic phase was washed with brine, dried over anhydrous Na₂SO₄, filtered and concentrated to dryness. The residue was purified by column chromatography on silica gel (PE:EA=5:1, V/V) to afford the desired product (385 mg, yield: 58%). LC/MS (ESI): m/z 406 [M+H]⁺.

### Step 2. (3R)-4-(7-((4-methoxybenzyl)oxy)-3-(1-(tetrahydro-2H-pyran-2-yl)-1H-pyrazol-5-yl)isothiazolo[4,5-b]pyridin-5-yl)-3-methylmorpholine

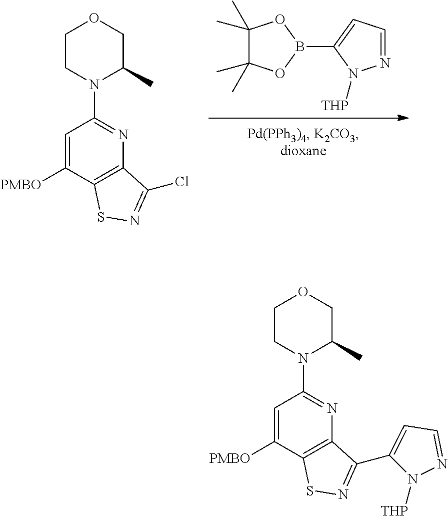

To a solution of (R)-4-(3-chloro-7-((4-methoxybenzyl)oxy)isothiazolo[4,5-b]pyridin-5-yl)-3-methylmorpholine (385 mg, 0.95 mmol) and 1-(oxan-2-yl)-5-(tetramethyl-1,3,2-dioxaborolan-2-yl)-1H-pyrazole (791 mg, 2.84 mmol) in dioxane (12 mL) were added K₂CO₃ (2M in H₂O, 2.4 mL, 4.74 mmol) and Pd(PPh₃)₄ (219 mg, 0.19 mmol). The mixture was stirred at 100° C. for 16 h under nitrogen atmosphere. LC-MS showed the reaction was complete. The reaction mixture was diluted with DCM (20 mL), then washed with water and brine, dried over anhydrous Na₂SO₄, filtered and concentrated. The residue was purified by column chromatography on silica gel (PE:EA=3:1, V/V) to afford the desired product (356 mg, yield: 72%). LC/MS (ESI): m/z 522 [M+H]⁺.

### Step 3. (R)-4-(7-chloro-3-(1H-pyrazol-5-yl)isothiazolo[4,5-b]pyridin-5-yl)-3-methyl morpholine

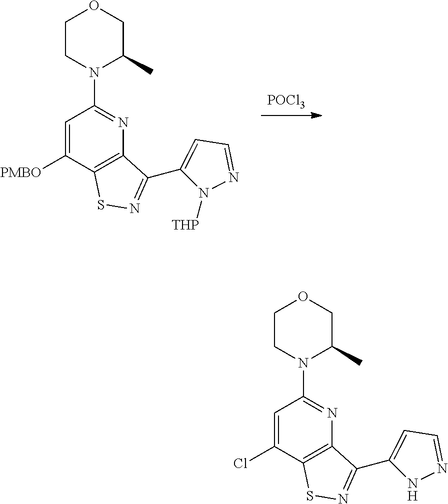

A mixture of (3R)-4-(7-((4-methoxybenzyl)oxy)-3-(1-(tetrahydro-2H-pyran-2-yl)-1H-pyrazol-5-yl)isothiazolo[4,5-b]pyridin-5-yl)-3-methylmorpholine (356 mg, 0.68 mmol) in POCl₃ (6 mL) was stirred at 100° C. for 3 h. LC-MS showed the reaction was complete. The reaction mixture was concentrated in vacuo to dryness, then diluted with DCM (40 mL). The resulting mixture was washed with saturated NaHCO₃ aqueous solution and brine, dried over anhydrous Na₂SO₄, filtered and concentrated. The residue was purified by column chromatography on silica gel (PE:EA=5:1, V/V) to afford the desired product (150 mg, yield: 65%). LC/MS (ESI): m/z 336 [M+H]⁺.

### Step 4. (3R)-4-(7-chloro-3-(1-(tetrahydro-2H-pyran-2-yl)-1H-pyrazol-5-yl) isothiazolo[4,5-b]pyridin-5-yl)-3-methylmorpholine

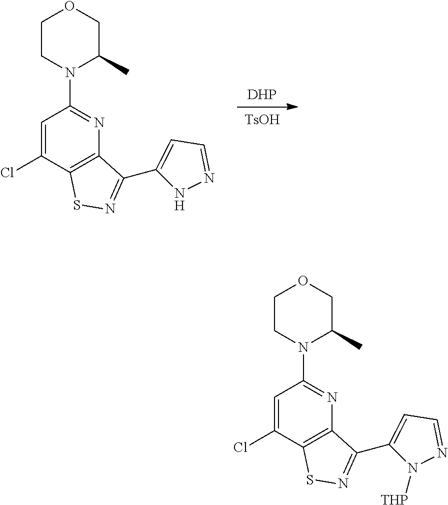

To a solution of (R)-4-(7-chloro-3-(1H-pyrazol-5-yl)isothiazolo[4,5-b]pyridin-5-yl)-3-methylmorpholine (150 mg, 0.45 mmol) and TsOH (15.4 mg, 0.09 mmol) in THF (6 mL) was added DHP (225 mg, 2.68 mmol). The mixture was stirred at 60° C. for 16 h. LC-MS showed the reaction was complete. The reaction mixture was diluted with DCM (30 mL), then washed with water and brine, dried over anhydrous Na₂SO₄, filtered and concentrated. The residue was purified by column chromatography on silica gel (PE:EA=3:1, V/V) to afford the desired product (90 mg, yield: 48%). LC/MS (ESI): m/z 420 [M+H]⁺.

### Step 5. methyl 5-((R)-3-methylmorpholino)-3-(1-(tetrahydro-2H-pyran-2-yl)-1H-pyrazol-5-yl)isothiazolo[4,5-b]pyridine-7-carboxylate

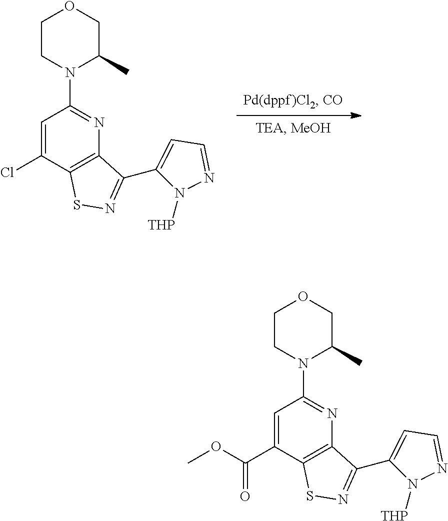

To a solution of (3R)-4-(7-chloro-3-(1-(tetrahydro-2H-pyran-2-yl)-1H-pyrazol-5-yl) isothiazolo[4,5-b]pyridin-5-yl)-3-methylmorpholine (90 mg, 0.22 mmol) and TEA (0.15 mL, 1.07 mmol) in MeOH (10 mL) was added Pd(dppf)Cl₂ (31 mg, 0.04 mmol). The mixture was stirred at 60° C. for 16 h under CO atmosphere. LC-MS showed the reaction was complete. The mixture was filtered, the filtrate was concentrated in vacuo to dryness. The residue was purified by column chromatography on silica gel (PE:EA=3:1, V/V) to afford the desired product (45 mg, yield: 47%). LC/MS (ESI): m/z 444 [M+H]⁺.

### Step 6. (5-((R)-3-methylmorpholino)-3-(1-(tetrahydro-2H-pyran-2-yl)-1H-pyrazol-5-yl)isothiazolo[4,5-b]pyridin-7-yl)methanol

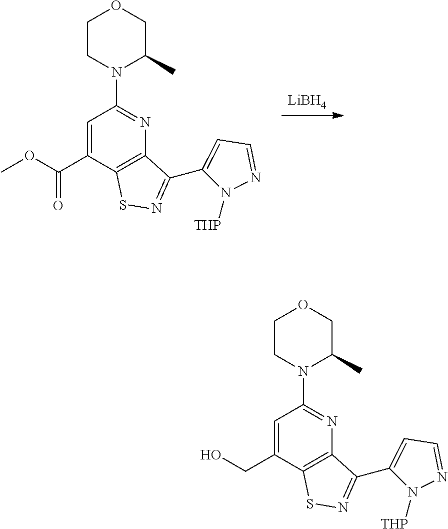

To a mixture of methyl methyl 5-((R)-3-methylmorpholino)-3-(1-(tetrahydro-2H-pyran-2-yl)-1H-pyrazol-5-yl)isothiazolo[4,5-b]pyridine-7-carboxylate (45 mg, 0.10 mmol) in THF (2 mL) at 0° C. was added LiBH₄ (2M in THF, 0.25 mL, 0.50 mmol) drop wise. The mixture was stirred at room temperature for 2 h. LC-MS showed the reaction was complete. The reaction mixture was quenched with saturated NH₄Cl aqueous solution, then extracted with EA (30 mL×2). The combined organic phase was washed with brine, dried over anhydrous Na₂SO₄, filtered and concentrated to dryness. The residue was purified by column chromatography on silica gel (PE:EA=1:1, V/V) to afford the desired product (32 mg, yield: 76%). LC/MS (ESI): m/z 416 [M+H]⁺.

### Step 7. (5-((R)-3-methylmorpholino)-3-(1-(tetrahydro-2H-pyran-2-yl)-1H-pyrazol-5-yl)isothiazolo[4,5-b]pyridin-7-yl)methyl methanesulfonate

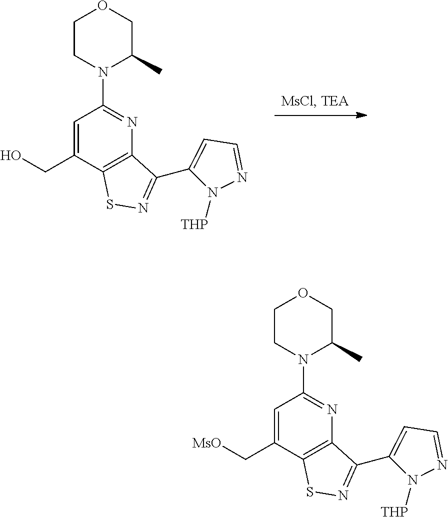

To a solution of (5-((R)-3-methylmorpholino)-3-(1-(tetrahydro-2H-pyran-2-yl)-1H-pyrazol-5-yl)isothiazolo[4,5-b]pyridin-7-yl)methanol (32 mg, 0.08 mmol) and TEA (0.03 mL, 0.23 mmol) in DCM (2 mL) at 0° C. was added MsCl (0.012 mL, 0.154 mmol) drop wise. The mixture was stirred at room temperature for 16 h. LC-MS showed the reaction was complete. The reaction mixture was diluted with DCM (30 mL), then washed with water and brine, dried over anhydrous Na₂SO₄, filtered and concentrated. The residue was purified by column chromatography on silica gel (PE:EA=3:1, V/V) to afford the desired product (25 mg, yield: 66%). LC/MS (ESI): m/z 494 [M+H]⁺.

### Step 8. (3R)-3-methyl-4-(7-((methylsulfonyl)methyl)-3-(1-(tetrahydro-2H-pyran-2-yl)-1H-pyrazol-5-yl)isothiazolo[4,5-b]pyridin-5-yl)morpholine

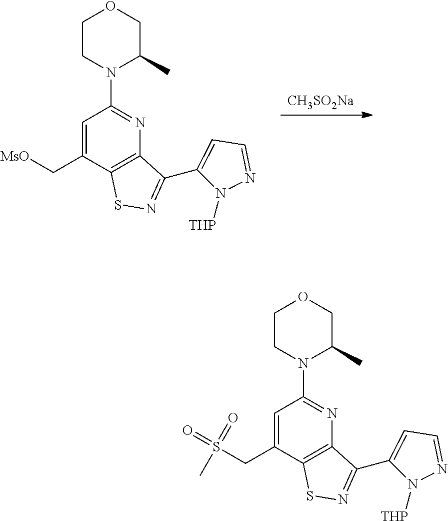

To a solution of (5-((R)-3-methylmorpholino)-3-(1-(tetrahydro-2H-pyran-2-yl)-1H-pyrazol-5-yl)isothiazolo[4,5-b]pyridin-7-yl)methyl methanesulfonate (25 mg, 0.05 mmol) in DMF (3 mL) was added CH₃SO₂Na (15.5 mg, 0.15 mmol). The mixture was stirred at room temperature for 16 h. LC-MS showed the reaction was complete. The reaction mixture was diluted with DCM (30 mL), then washed with water and brine, dried over anhydrous Na₂SO₄, filtered and concentrated. The residue was purified by column chromatography on silica gel (PE:EA=3:1, V/V) to afford the desired product (22 mg, yield: 91%). LC/MS (ESI): m/z 478 [M+H]⁺.

### Step 9. (3R)-3-methyl-4-(7-(1-(methylsulfonyl)cyclopropyl)-3-(1-(tetrahydro-2H-pyran-2-yl)-1H-pyrazol-5-yl)isothiazolo[4,5-b]pyridin-5-yl)morpholine

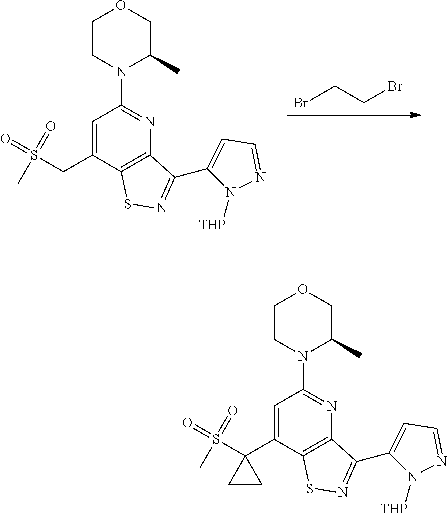

To a solution of (3R)-3-methyl-4-(7-((methylsulfonyl)methyl)-3-(1-(tetrahydro-2H-pyran-2-yl)-1H-pyrazol-5-yl)isothiazolo[4,5-b]pyridin-5-yl)morpholine (22 mg, 0.05 mmol), 1,2-dibromoethane (0.02 mL, 0.23 mmol) and TBAB (3 mg, 0.01 mmol) in Toluene (5 mL) was added NaOH (10M in H₂O, 0.05 mL, 0.46 mmol). The mixture was stirred at 60° C. for 3 h. LC-MS showed the reaction was complete. The reaction mixture was diluted with DCM (30 mL), then washed with water and brine, dried over anhydrous Na₂SO₄, filtered and concentrated. The residue was purified by column chromatography on silica gel (PE:EA=1:1, V/V) to afford the desired product (20 mg, yield: 86%). LC/MS (ESI): m/z 504 [M+H]⁺.

### Step 10. (R)-3-methyl-4-(7-(1-(methylsulfonyl)cyclopropyl)-3-(1H-pyrazol-5-yl) isothiazolo[4,5-b]pyridin-5-yl)morpholine

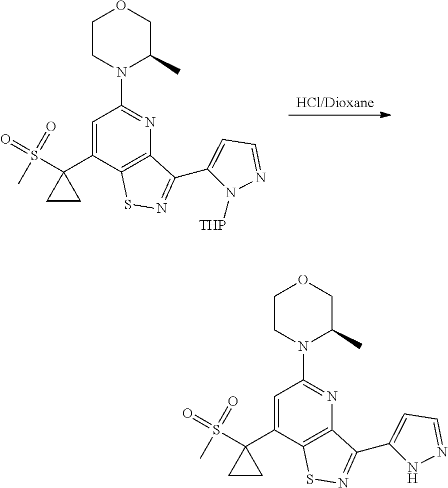

To a solution of (3R)-3-methyl-4-(7-(1-(methylsulfonyl)cyclopropyl)-3-(1-(tetrahydro-2H-pyran-2-yl)-1H-pyrazol-5-yl)isothiazolo[4,5-b]pyridin-5-yl) morpholine (20 mg, 0.04 mmol) in DCM (1 mL) was added HCl solution (4M in dioxane, 1 mL). The mixture was stirred at room temperature for 1 h. LC-MS showed the reaction was complete. The reaction mixture was concentrated in vacuo. The residue was purified by Pre-HPLC (C₁₈, 10-95%, MeOH in H₂O with 0.1% TFA) to afford the desired product (3.4 mg, yield: 20%). LC/MS (ESI) m/z: 420 [M+H]⁺. ¹H NMR (400 MHz, DMSO) δ 13.57 (d, J=169.7 Hz, 1H), 7.87 (d, J=83.3 Hz, 1H), 7.59 (s, 1H), 7.47 (d, J=1.8 Hz, 1H), 4.63 (dd, J=12.9, 6.8 Hz, 1H), 4.24 (d, J=13.2 Hz, 1H), 4.14 (dd, J=11.6, 3.0 Hz, 1H), 3.92 (d, J=11.2 Hz, 1H), 3.81 (dd, J=11.4, 2.7 Hz, 1H), 3.66 (td, J=11.8, 2.8 Hz, 1H), 3.38-3.30 (m, 1H), 3.17 (s, 3H), 1.91-1.83 (m, 2H), 1.67-1.58 (m, 2H), 1.34 (d, J=6.6 Hz, 3H).

### Example 50

### Synthesis of (R)-3-methyl-4-(3-(3-methyl-1H-pyrazol-5-yl)-7-(1-(methylsulfonyl)cyclopropyl)isothiazolo[4,5-b]pyridin-5-yl)morpholine

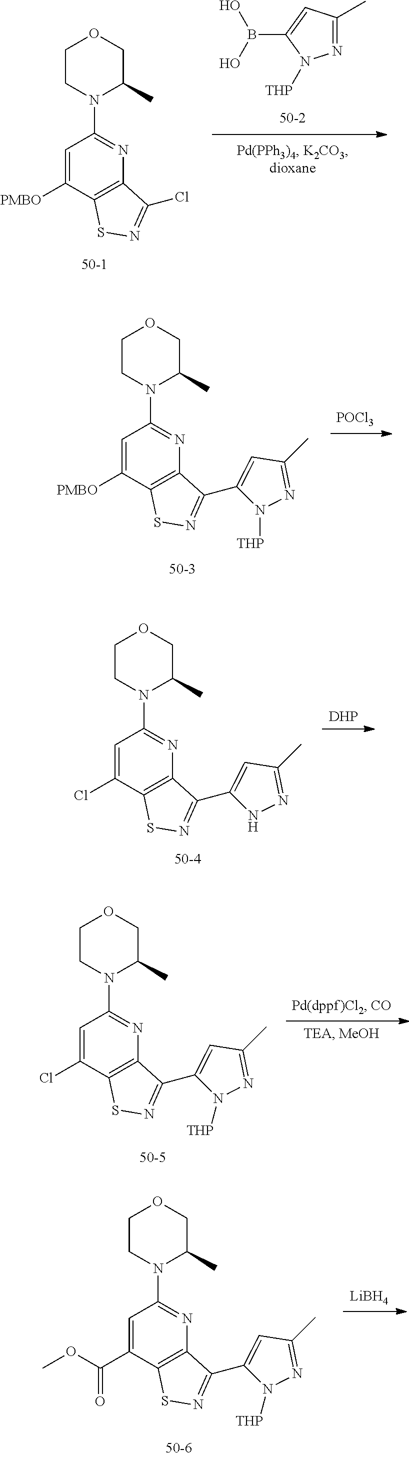

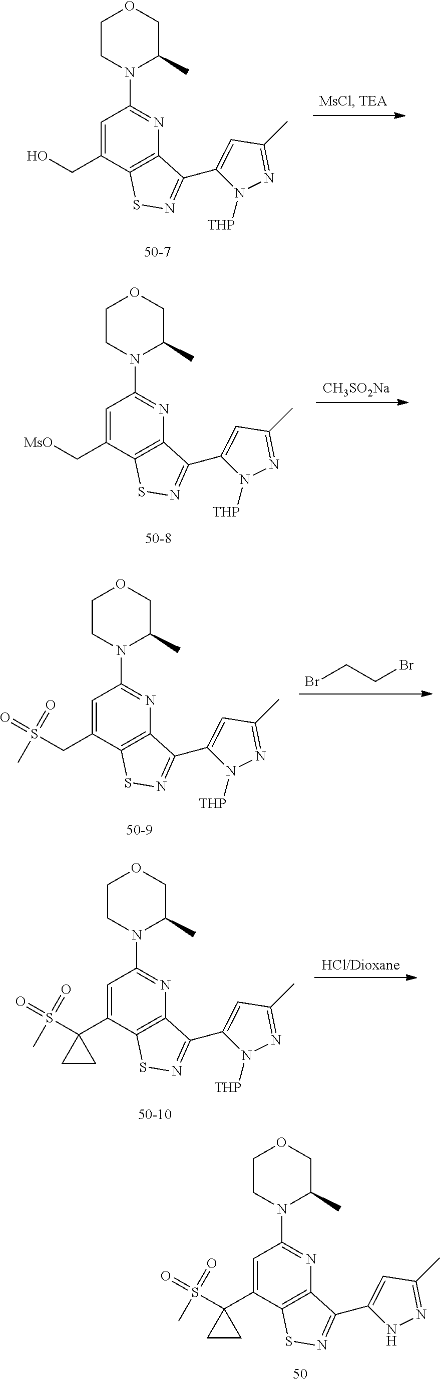

### Step 1. (3R)-4-(7-((4-methoxybenzyl)oxy)-3-(3-methyl-1-(tetrahydro-2H-pyran-2-yl)-1H-pyrazol-5-yl)isothiazolo[4,5-b]pyridin-5-yl)-3-methylmorpholine

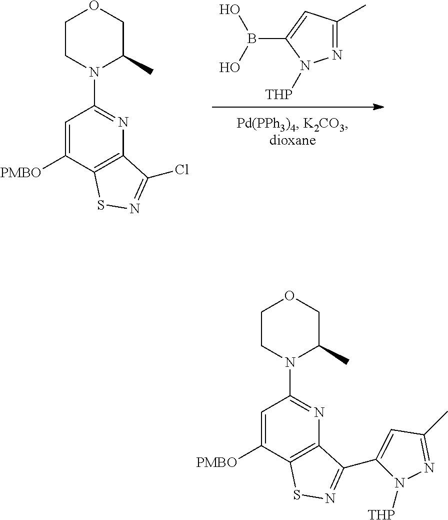

To a mixture of (R)-4-(3-chloro-7-((4-methoxybenzyl)oxy)isothiazolo[4,5-b]pyridin-5-yl)-3-methylmorpholine (500 mg, 1.23 mmol), [3-methyl-1-(oxan-2-yl)-1H-pyrazol-5-yl]boronic acid (776 mg, 3.70 mmol) and K₂CO₃ (2M in H₂O, 3.1 mL, 6.16 mmol) in dioxane (15 mL) was added Pd(PPh₃)₄ (285 mg, 0.25 mmol). The mixture was stirred at 100° C. for 16 h under N₂ atmosphere. LC-MS showed the reaction was complete. The mixture was diluted with EA (50 mL), then washed with water and brine, dried over anhydrous Na₂SO₄, filtered and concentrated. The residue was purified by flash chromatography on silica gel (PE:EA=3:1, V/V) to afford the desired product (484 mg, yield: 73%). LC/MS (ESI): m/z 536 [M+H]⁺.

### Step 2. (R)-4-(7-chloro-3-(3-methyl-1H-pyrazol-5-yl)isothiazolo[4,5-b]pyridin-5-yl)-3-methylmorpholine

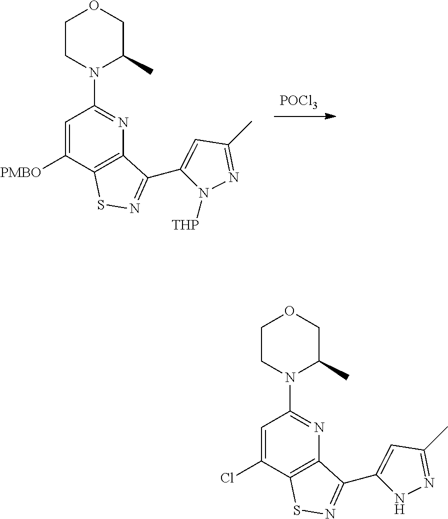

To a mixture of (3R)-4-(7-((4-methoxybenzyl)oxy)-3-(3-methyl-1-(tetrahydro-2H-pyran-2-yl)-1H-pyrazol-5-yl)isothiazolo[4,5-b]pyridin-5-yl)-3-methylmorpholine (484 mg, 0.90 mmol) in POCl₃ (10 mL) was stirred at 100° C. for 3 h. LC-MS showed the reaction was complete. The mixture was concentrated under reduced pressure to dryness. The residue was diluted with DCM (40 mL), then washed with saturated NaHCO₃ aqueous solution and brine, dried over anhydrous Na₂SO₄, filtered and concentrated. The residue was purified by column chromatography on silica gel (PE:EA=5:1, V/V) to afford the desired product (282 mg, yield: 89%). LC/MS (ESI): m/z 350 [M+H]⁺.

### Step 3. (3R)-4-(7-chloro-3-(3-methyl-1-(tetrahydro-2H-pyran-2-yl)-1H-pyrazol-5-yl)isothiazolo[4,5-b]pyridin-5-yl)-3-methylmorpholine

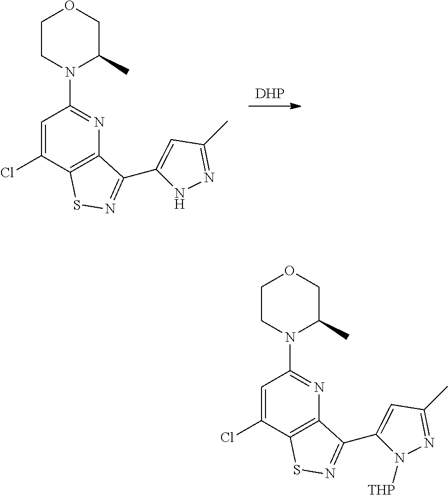

To a solution of (R)-4-(7-chloro-3-(3-methyl-1H-pyrazol-5-yl)isothiazolo[4,5-b] pyridin-5-yl)-3-methylmorpholine (282 mg, 0.81 mmol) and TsOH (28 mg, 0.16 mmol) in THF (10 mL) was added DHP (406 mg, 4.84 mmol). The mixture was stirred at 60° C. for 16 h. LC-MS showed the reaction was complete. The mixture was diluted with EA (50 mL), then washed with water and brine, dried over anhydrous Na₂SO₄, filtered and concentrated. The residue was purified by column chromatography on silica gel (PE:EA=3:1, V/V) to afford the desired product (200 mg, yield: 57%). LC/MS (ESI): m/z 434 [M+H]⁺.

### Step 4. methyl 3-(3-methyl-1-(tetrahydro-2H-pyran-2-yl)-1H-pyrazol-5-yl)-5-((R)-3-methylmorpholino)isothiazolo[4,5-b]pyridine-7-carboxylate

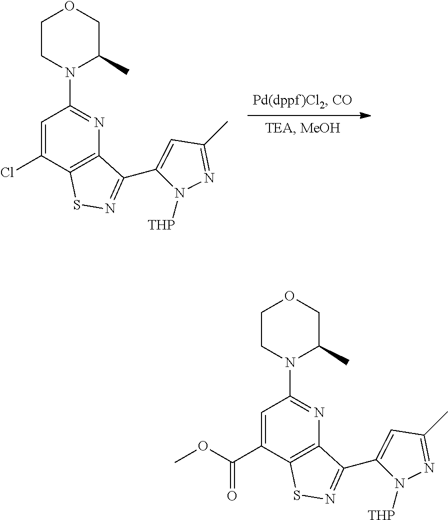

To a mixture of (3R)-4-(7-chloro-3-(3-methyl-1-(tetrahydro-2H-pyran-2-yl)-1H-pyrazol-5-yl)isothiazolo[4,5-b]pyridin-5-yl)-3-methylmorpholine (200 mg, 0.46 mmol) and TEA (0.64 mL, 4.61 mmol) in MeOH (10 mL) was added Pd(dppf)Cl₂ (67 mg, 0.09 mmol). The mixture was stirred at 60° C. for 16 h under CO atmosphere. LC-MS showed the reaction was complete. The mixture was filtered and concentrated to dryness. The residue was purified by column chromatography on silica gel (PE:EA=3:1, V/V) to afford the desired product (110 mg, yield: 52%). LC/MS (ESI): m/z 458 [M+H]⁺.

### Step 5. (3-(3-methyl-1-(tetrahydro-2H-pyran-2-yl)-1H-pyrazol-5-yl)-5-((R)-3-methylmorpholino)isothiazolo[4,5-b]pyridin-7-yl)methanol

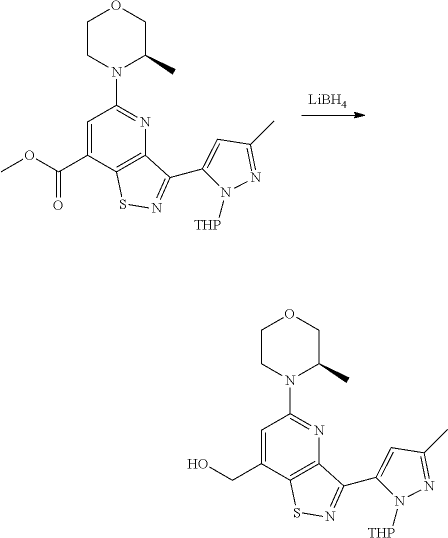

To a solution of methyl 3-(3-methyl-1-(tetrahydro-2H-pyran-2-yl)-1H-pyrazol-5-yl)-5-((R)-3-methylmorpholino)isothiazolo[4,5-b]pyridine-7-carboxylate (110 mg, 0.24 mmol) in THF (5 mL) at 0° C. was added LiBH₄ (2M in THF, 0.6 mL, 1.20 mmol). The mixture was stirred at room temperature for 2 h. LC-MS showed the reaction was complete. The mixture was diluted with EA (50 mL), then washed with water and brine, dried over anhydrous Na₂SO₄, filtered and concentrated. The residue was purified by column chromatography on silica gel (PE:EA=3:1, V/V) to afford the desired product (82 mg, yield: 79%). LC/MS (ESI): m/z 430 [M+H]⁺.

### Step 6. (3-(3-methyl-1-(tetrahydro-2H-pyran-2-yl)-1H-pyrazol-5-yl)-5-((R)-3-methyl morpholino)isothiazolo[4,5-b]pyridin-7-yl)methyl methanesulfonate

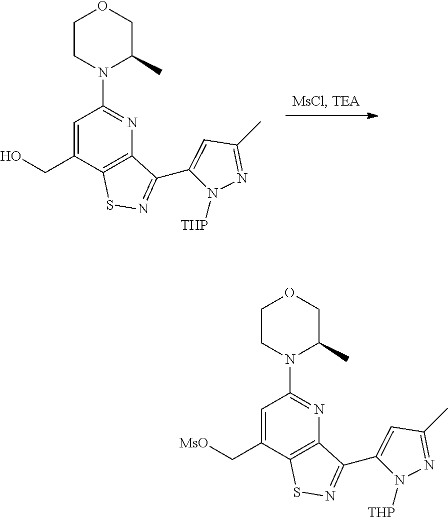

To a mixture of (3-(3-methyl-1-(tetrahydro-2H-pyran-2-yl)-1H-pyrazol-5-yl)-5-((R)-3-methylmorpholino)isothiazolo[4,5-b]pyridin-7-yl)methanol (82 mg, 0.19 mmol) and TEA (0.08 mL, 0.57 mmol) in DCM (5 mL) at 0° C. was added MsCl (0.03 mL, 0.38 mmol). The mixture was stirred at room temperature for 6 h. LC-MS showed the reaction was complete. LC-MS showed the reaction was complete. The mixture was diluted with DCM (30 mL), then washed with water and brine, dried over anhydrous Na₂SO₄, filtered and concentrated. The residue was purified by column chromatography on silica gel (PE:EA=5:1, V/V) to afford the desired product (70 mg, yield: 72%). LC/MS (ESI): m/z 508 [M+H]⁺.

### Step 7. (3R)-3-methyl-4-(3-(3-methyl-1-(tetrahydro-2H-pyran-2-yl)-1H-pyrazol-5-yl)-7-((methylsulfonyl)methyl)isothiazolo[4,5-b]pyridin-5-yl)morpholine

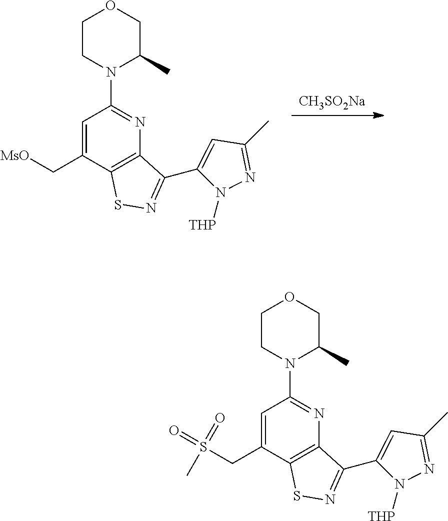

To a mixture of (3-(3-methyl-1-(tetrahydro-2H-pyran-2-yl)-1H-pyrazol-5-yl)-5-((R)-3-methylmorpholino)isothiazolo[4,5-b]pyridin-7-yl)methyl methanesulfonate (70 mg, 0.14 mmol) in DMF (3 mL) was added CH₃SO₂Na (42 mg, 0.41 mmol). The mixture was stirred at 40° C. for 16 h. LC-MS showed the reaction was complete. The mixture was diluted with EA (40 mL), then washed with water and brine, dried over anhydrous Na₂SO₄, filtered and concentrated. The residue was purified by column chromatography on silica gel (PE:EA=3:1, V/V) to afford the desired product (54 mg, yield: 80%). LC/MS (ESI): m/z 492 [M+H]⁺.

### Step 8. (3R)-3-methyl-4-(3-(3-methyl-1-(tetrahydro-2H-pyran-2-yl)-1H-pyrazol-5-yl)-7-(1-(methylsulfonyl)cyclopropyl)isothiazolo[4,5-b]pyridin-5-yl)morpholine

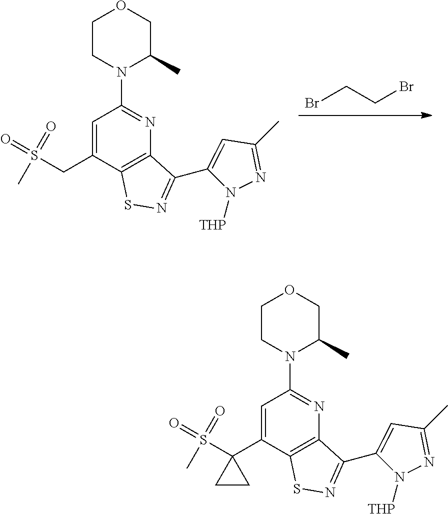

To a solution of (3R)-3-methyl-4-(3-(3-methyl-1-(tetrahydro-2H-pyran-2-yl)-1H-pyrazol-5-yl)-7-((methylsulfonyl)methyl)isothiazolo[4,5-b]pyridin-5-yl)morpholine (24 mg, 0.05 mmol), 1,2-dibromoethane (0.02 mL, 0.25 mmol) and TBAB (3.15 mg, 0.01 mmol) in Toluene (3 mL) was added NaOH (10 M in H₂O, 0.05 mL, 0.5 mmol). The mixture was stirred at 60° C. for 3 h. LC-MS showed the reaction was complete. LC-MS showed the reaction was complete. The mixture was diluted with EA (40 mL), then washed with water and brine, dried over anhydrous Na₂SO₄, filtered and concentrated. The residue was purified by column chromatography on silica gel (PE:EA=3:1, V/V) to afford the desired product (21 mg, yield: 83%). LC/MS (ESI): m/z 518 [M+H]⁺.

### Step 9. (R)-3-methyl-4-(3-(3-methyl-1H-pyrazol-5-yl)-7-(1-(methylsulfonyl)cyclo propyl)isothiazolo[4,5-b]pyridin-5-yl)morpholine

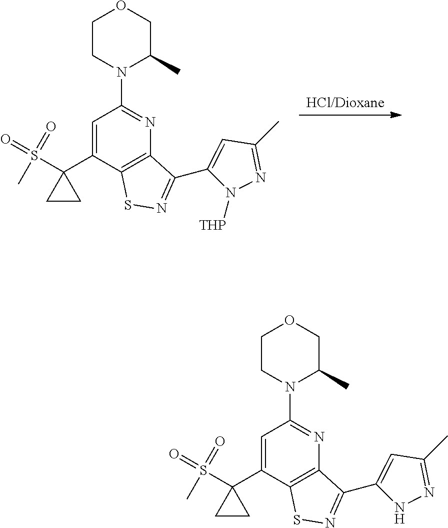

To a solution of (3R)-3-methyl-4-(3-(3-methyl-1-(tetrahydro-2H-pyran-2-yl)-1H-pyrazol-5-yl)-7-(1-(methylsulfonyl)cyclopropyl)isothiazolo[4,5-b]pyridin-5-yl) morpholine (21 mg, 0.04 mmol) in DCM (1.0 mL) was added HCl solution (4M in dioxane, 1.0 mL). The mixture was stirred at room temperature for 1 h. LC-MS showed the reaction was complete. The mixture was concentrated in vacuo to dryness. The residue was purified by Prep-HPLC (C₁₈, 10-95%, MeCN in H₂O with 0.1% HCOOH) to give the desired product (6 mg, yield: 34%). LC/MS (ESI): m/z 434 [M+H]⁺. ¹H NMR (400 MHz, DMSO) δ 13.07 (d, J=118.7 Hz, 1H), 7.49 (s, 1H), 7.10 (s, 1H), 4.53 (dd, J=15.0, 6.6 Hz, 1H), 4.14 (d, J=13.5 Hz, 1H), 4.04 (dd, J=11.3, 2.9 Hz, 1H), 3.82 (d, J=11.3 Hz, 1H), 3.72 (dd, J=11.4, 2.8 Hz, 1H), 3.57 (td, J=11.8, 2.7 Hz, 1H), 3.24 (dd, J=12.7, 3.5 Hz, 1H), 3.07 (s, 3H), 2.30 (s, 3H), 1.77 (q, J=4.3 Hz, 2H), 1.56-1.49 (m, 2H), 1.24 (d, J=6.6 Hz, 3H).

### Example 51

### Synthesis of (R)-1-(5-(3-methylmorpholino)-3-(1H-pyrazol-5-yl)isothiazolo[4,5-b]pyridin-7-yl)cyclopropane-1-carbonitrile

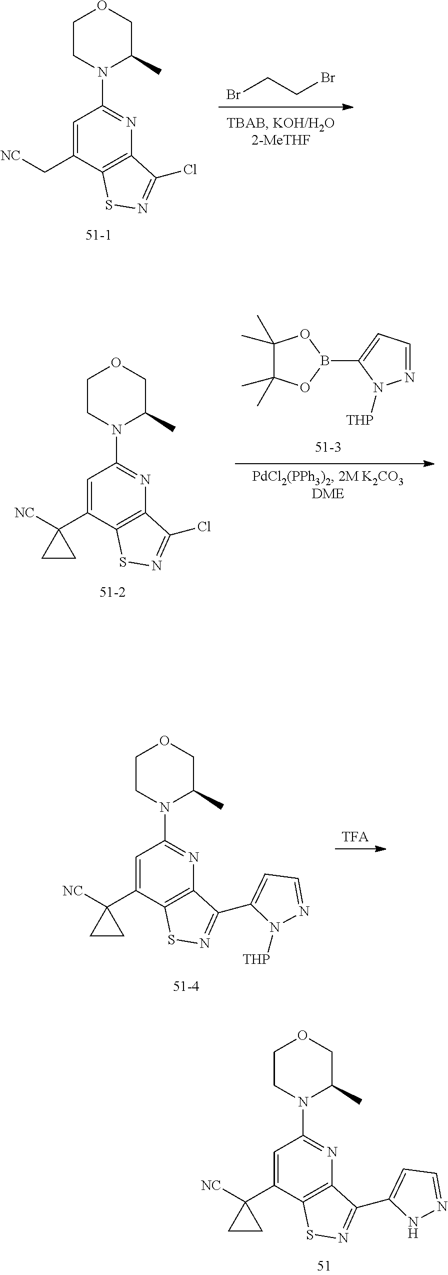

### Step 1. (R)-1-(3-chloro-5-(3-methylmorpholino)isothiazolo[4,5-b]pyridin-7-yl)cyclopropane-1-carbonitrile

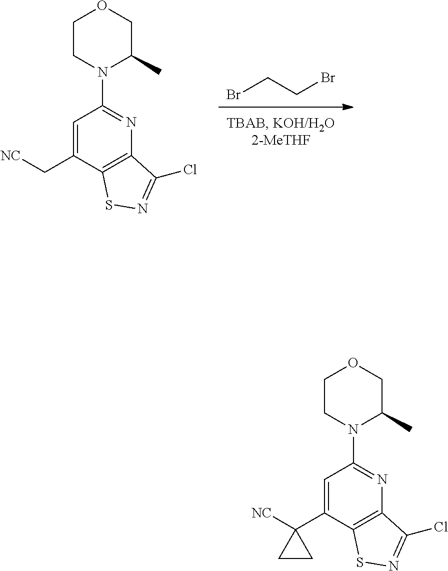

A mixture of 2-{3-chloro-5-[(3R)-3-methylmorpholin-4-yl]-[1,2]thiazolo[4,5-b] pyridin-7-yl}acetonitrile (30 mg, 0.09 mmol), 1,2-dibromoethane (73 mg, 0.38 mmol), TBAB (6 mg, 0.02 mmol) and KOH (10.0 M in H₂O, 0.2 mL, 1.9 mmol) in 2-Methyltetrahydrofuran (3 mL) was stirred at 70° C. for 4 h. LC-MS showed the reaction was complete. The reaction mixture was diluted with EA (40 mL), then washed with water and brine, dried over anhydrous Na₂SO₄, filtered and concentrated. The residue was purified by column chromatography on silica gel (PE:EA=2:1, V/V) to afford the desired product (26 mg, yield: 81%). LC/MS (ESI): m/z 335 [M+H]⁺.

### Step 2. 1-(5-((R)-3-methylmorpholino)-3-(1-(tetrahydro-2H-pyran-2-yl)-1H-pyrazol-5-yl)isothiazolo[4,5-b]pyridin-7-yl)cyclopropane-1-carbonitrile

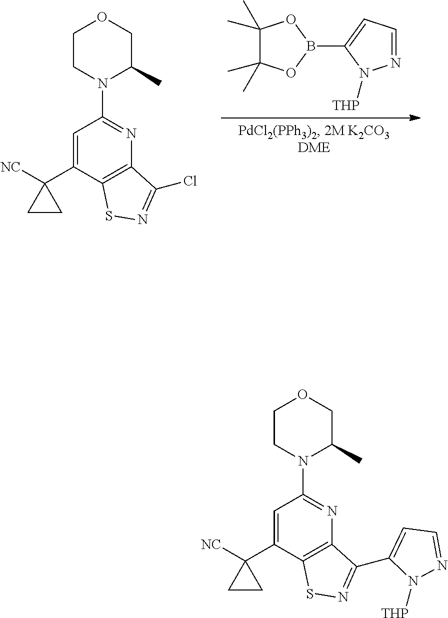

A mixture of 1-{3-chloro-5-[(3R)-3-methylmorpholin-4-yl]-[1,2]thiazolo[4,5-b] pyridin-7-yl}cyclopropane-1-carbonitrile (30 mg, 0.09 mmol), 1-(oxan-2-yl)-5-(tetramethyl-1,3,2-dioxaborolan-2-yl)-1H-pyrazole (50 mg, 0.18 mmol), Pd(dppf)Cl₂ (13 mg, 0.02 mmol) and K₂CO₃ (2.0 M in H₂O, 0.13 mL, 0.26 mmol) in dioxane (1 mL) was stirred at 100° C. for 16 h under N₂ atmosphere. LC-MS showed the reaction was complete. The mixture was diluted with EA (10 mL), then washed with water and brine, dried over anhydrous Na₂SO₄, filtered and concentrated. The residue was purified by flash chromatography on silica gel (DCM:MeOH=20:1, V/V) to afford the desired product (15 mg, yield: 37%). LC/MS (ESI): m/z 451 [M+H]⁺.

### Step 3. (R)-1-(5-(3-methylmorpholino)-3-(1H-pyrazol-5-yl)isothiazolo[4,5-b]pyridin-7-yl)cyclopropane-1-carbonitrile

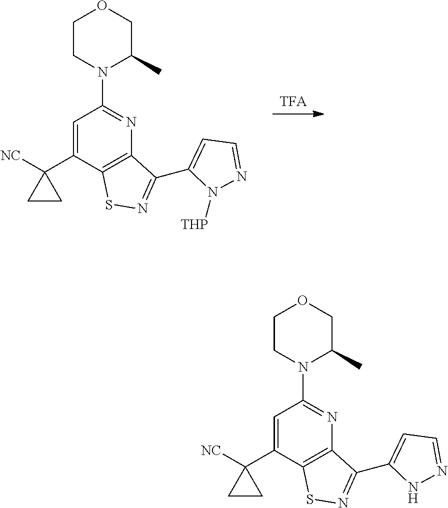

A mixture of 1-{5-[(3R)-3-methylmorpholin-4-yl]-3-[1-(oxan-2-yl)-1H-pyrazol-5-yl]-[1,2]thiazolo[4,5-b]pyridin-7-yl}cyclopropane-1-carbonitrile (15 mg, 0.03 mmol) in TFA (2.0 mL) was stirred at room temperature for 2 h. LC-MS showed the reaction was complete. The reaction mixture was concentrated under reduced pressure. The residue was purified by Prep-HPLC (C18, 10-95%, MeOH in H₂O with 0.1% HCOOH) to give the desired product (5 mg, yield: 40%). LC/MS (ESI): m/z 367 [M+H]⁺. ¹H NMR (400 MHz, DMSO) δ 13.51 (d, J=175.8 Hz, 1H), 7.79 (d, J=88.0 Hz, 1H), 7.38 (d, J=1.8 Hz, 1H), 7.14 (s, 1H), 4.58 (s, 1H), 4.07 (dd, J=42.5, 10.4 Hz, 2H), 3.80 (d, J=11.3 Hz, 1H), 3.68 (dd, J=11.4, 2.7 Hz, 1H), 3.53 (td, J=11.8, 2.7 Hz, 1H), 3.28-3.17 (m, 1H), 1.93-1.72 (m, 4H), 1.22 (d, J=6.6 Hz, 3H).

### Example 52

### Synthesis of (R)-1-(3-(3-methyl-1H-pyrazol-5-yl)-5-(3-methylmorpholino)isothiazolo [4,5-b]pyridin-7-yl)cyclopropane-1-carbonitrile

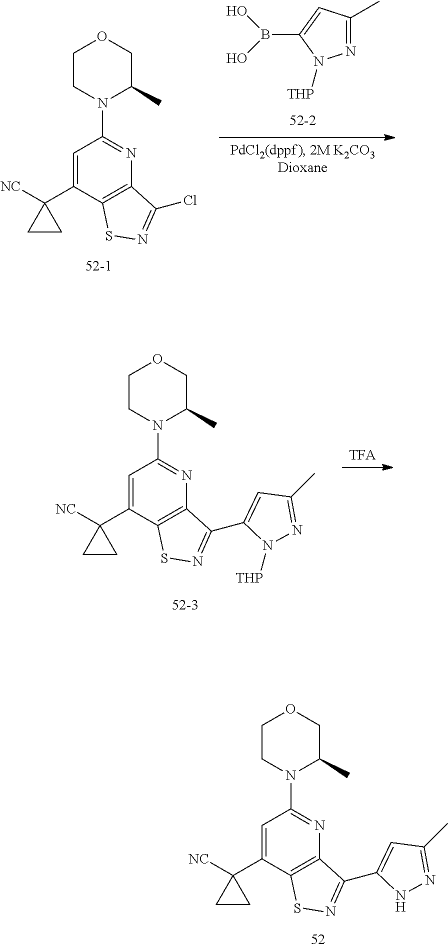

### Step 1. 1-(3-(3-methyl-1-(tetrahydro-2H-pyran-2-yl)-1H-pyrazol-5-yl)-5-((R)-3-methylmorpholino)isothiazolo[4,5-b]pyridin-7-yl)cyclopropane-1-carbonitrile

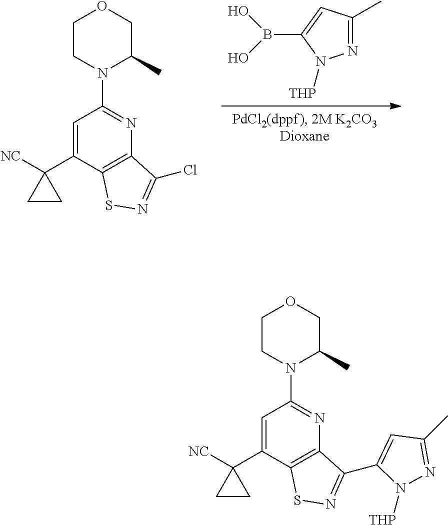

A mixture of 1-{3-chloro-5-[(3R)-3-methylmorpholin-4-yl]-[1,2]thiazolo[4,5-b] pyridin-7-yl}cyclopropane-1-carbonitrile (55 mg, 0.16 mmol), [3-methyl-1-(oxan-2-yl)-1H-pyrazol-5-yl]boronic acid (103 mg, 0.49 mmol), Pd(dppf)Cl₂ (24 mg, 0.03 mmol) and K₂CO₃ (2.0 M in H₂O, 0.25 mL, 0.50 mmol) in dioxane (3 mL) was stirred at 100° C. for 16 h under N₂ atmosphere. LC-MS showed the reaction was complete. The mixture was diluted with EA (30 mL), then washed with water and brine, dried over anhydrous Na₂SO₄, filtered and concentrated. The residue was purified by flash chromatography on silica gel (DCM:MeOH=20:1, V/V) to afford the desired product (40 mg, yield: 52%). LC/MS (ESI): m/z 465 [M+H]⁺.

### Step 2. (R)-1-(3-(3-methyl-1H-pyrazol-5-yl)-5-(3-methylmorpholino) isothiazolo[4,5-b]pyridin-7-yl)cyclopropane-1-carbonitrile

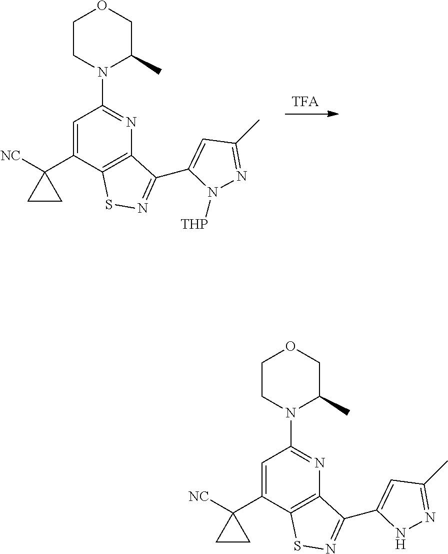

A mixture of 1-{3-[3-methyl-1-(oxan-2-yl)-1H-pyrazol-5-yl]-5-[(3S)-3-methyl morpholin-4-yl]-[1,2]thiazolo[4,5-b]pyridin-7-yl}cyclopropane-1-carbonitrile (40 mg, 0.08 mmol) in TFA (4.0 mL) was stirred at 25° C. for 2 h. LC-MS showed the reaction was complete. The reaction mixture was concentrated under reduced pressure. The residue was purified by Prep-HPLC (C18, 10-95%, MeOH in H₂O with 0.1% HCOOH) to give the desired product (10 mg, yield: 30%). LC/MS (ESI): m/z 381 [M+H]⁺. ¹H NMR (400 MHz, DMSO) δ 13.10 (d, J=125.6 Hz, 1H), 7.13 (s, 2H), 4.56 (s, 1H), 4.13 (d, J=12.6 Hz, 1H), 4.02 (d, J=11.1 Hz, 1H), 3.81 (d, J=11.4 Hz, 1H), 3.69 (dd, J=11.4, 2.8 Hz, 1H), 3.54 (dt, J=11.8, 6.0 Hz, 1H), 3.26 (d, J=11.8 Hz, 1H), 2.32 (d, J=19.7 Hz, 3H), 1.83 (dd, J=29.1, 8.6 Hz, 4H), 1.23 (d, J=6.7 Hz, 3H).

### Example 53

### Synthesis of (R)-2-methyl-2-(5-(3-methylmorpholino)-3-(1H-pyrazol-5-yl) isothiazolo [4,5-b]pyridin-7-yl)propanenitrile

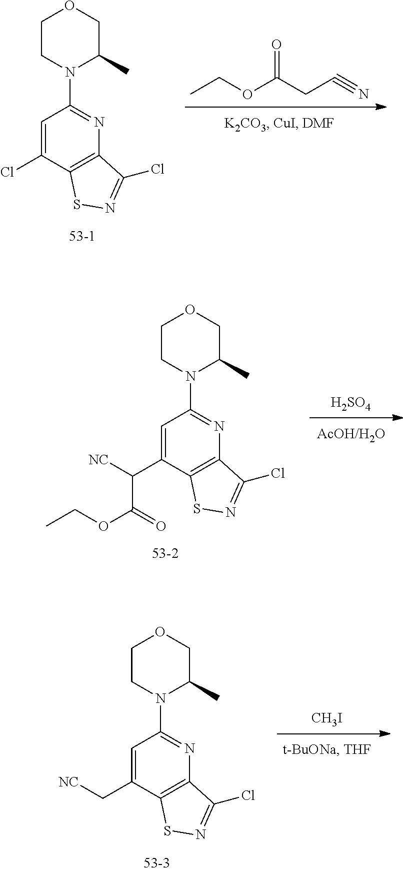

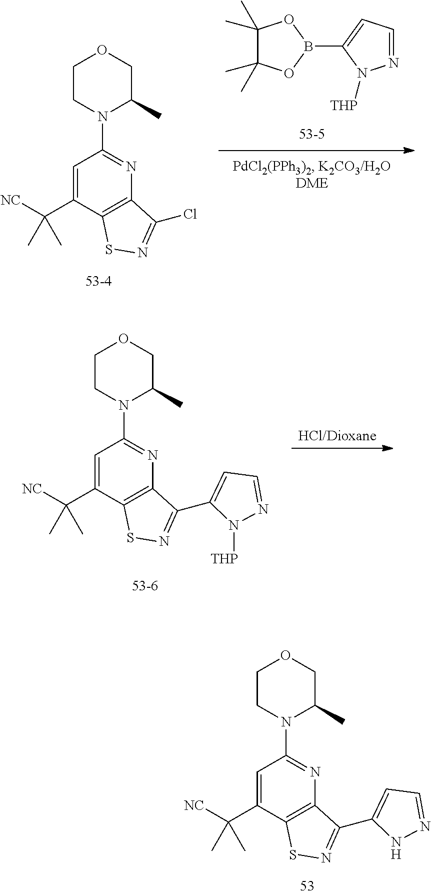

### Step 1. ethyl 2-(3-chloro-5-((R)-3-methylmorpholino)isothiazolo[4,5-b]pyridin-7-yl)-2-cyanoacetate

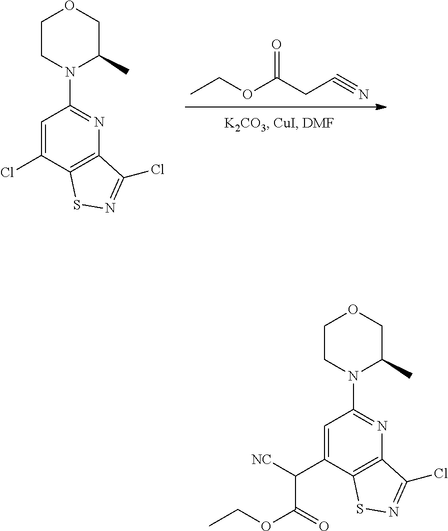

A mixture of (3R)-4-{3,7-dichloro-[1,2]thiazolo[4,5-b]pyridin-5-yl}-3-methyl morpholine (100 mg, 0.33 mmol), ethyl 2-cyanoacetate (74 mg, 0.65 mmol), K₂CO₃ (136 mg, 0.98 mmol) and CuI (12 mg, 0.06 mmol) in anhydrous DMF (2 mL) was stirred at 100° C. for 16 h under N₂ atmosphere. LC-MS showed the reaction was complete. The reaction mixture was diluted with DCM (20 mL), then washed with water and brine, dried over anhydrous Na₂SO₄, filtered and concentrated. The residue was purified by column chromatography on silica gel (DCM:MeOH=40:1, V/V) to afford the desired product (100 mg, yield: 79%). LC/MS (ESI): m/z 381 [M+H]⁺. Step 2. (R)-2-(3-chloro-5-(3-methylmorpholino)isothiazolo[4,5-b]pyridin-7-yl) acetonitrile

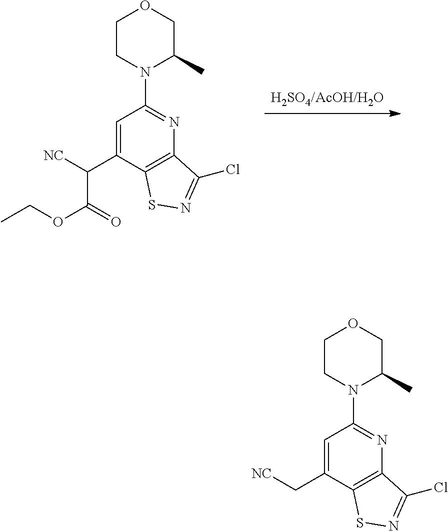 [SMILES: CCOC(=O)C(C#N)c1cc(N2CCOC[C@H]2C)nc2c(Cl)nsc12 ; "H2SO4/AcOH/H2O" ; C[C@@H]1COCCN1c1cc(CC#N)c2snc(Cl)c2n1]

To a solution of ethyl 2-{3-chloro-5-[(3R)-3-methylmorpholin-4-yl]-[1,2]thiazolo [4,5-b]pyridin-7-yl}-2-cyanoacetate (100 mg, 0.26 mmol) in co-solvent of AcOH (2 mL) and H₂O (2 mL) was added H₂SO₄ (0.2 mL). The resulting mixture was stirred at 120° C. for 2 h. LC-MS showed the reaction was complete. The reaction mixture was diluted with DCM (30 mL), then washed with saturated NaHCO₃ aqueous solution and brine, dried over anhydrous Na₂SO₄, filtered and concentrated.

The residue was purified by column chromatography on silica gel (PE:EA=2:1, V/V) to afford the desired product (67 mg, yield: 82%). LC/MS (ESI): m/z 309 [M+H]⁺.

### Step 3. (R)-2-(3-chloro-5-(3-methylmorpholino)isothiazolo[4,5-b]pyridin-7-yl)-2-methylpropanenitrile

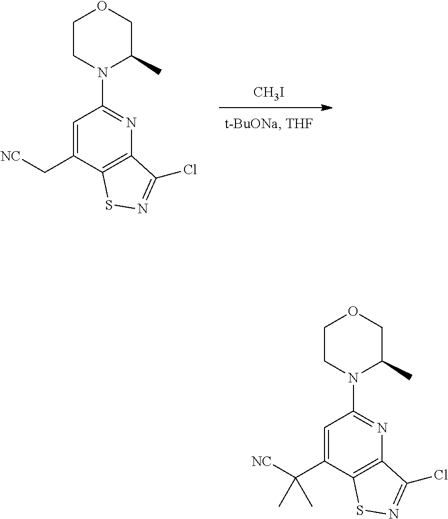 [SMILES: C[C@@H]1COCCN1c1cc(CC#N)c2snc(Cl)c2n1 ; "CH3I" ; C[C@@H]1COCCN1c1cc(C(C)(C)C#N)c2snc(Cl)c2n1]

To a solution of 2-{3-chloro-5-[(3R)-3-methylmorpholin-4-yl]-[1,2]thiazolo[4,5-b]pyridin-7-yl}acetonitrile (18 mg, 0.05 mmol) and t-BuONa (11 mg, 0.11 mmol) in anhydrous DMF (1 mL) at 0° C. was added a solution of CH₃I (16 mg, 0.11 mmol) in anhydrous DMF (0.5 mL) drop wise. After the addition, the resulting mixture was stirred at room temperature for 1 h. LC-MS showed the reaction was complete. The reaction mixture was diluted with EA (40 mL), then washed with water and brine, dried over anhydrous Na₂SO₄, filtered and concentrated.

The residue was purified by column chromatography on silica gel (PE:EA=2:1, V/V) to afford the desired product (10 mg, yield: 50%). LC/MS (ESI): m/z 337 [M+H]⁺.

### Step 4. 2-methyl-2-(5-((R)-3-methylmorpholino)-3-(1-(tetrahydro-2H-pyran-2-yl)-1H-pyrazol-5-yl)isothiazolo[4,5-b]pyridin-7-yl)propanenitrile

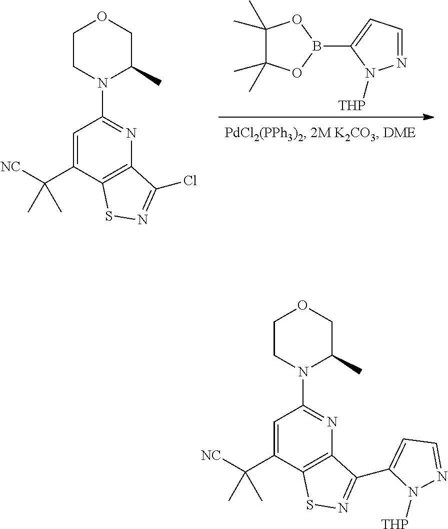

A mixture of 2-{3-chloro-5-[(3R)-3-methylmorpholin-4-yl]-[1,2]thiazolo[4,5-b] pyridin-7-yl}-2-methylpropanenitrile (38 mg, 0.11 mmol), 1-(oxan-2-yl)-5-(tetra methyl-1,3,2-dioxaborolan-2-yl)-1H-pyrazole (63 mg, 0.22 mmol), Pd(dppf)Cl₂ (16 mg, 0.02 mmol) and K₂CO₃ (2.0 M in H₂O, 0.17 mL, 0.34 mmol) in Dioxane (1.5 mL) was stirred at 100° C. for 16 h under N₂ atmosphere. LC-MS showed the reaction was complete. The reaction mixture was diluted with H₂O (20 mL), then extracted with EA (20 mL×3). The combined organic layer was washed with brine, dried over anhydrous Na₂SO₄, filtered and concentrated. The residue was purified by column chromatography on silica gel (DCM:MeOH=20:1, V/V) to give the desired product (30 mg, yield: 58%). LC/MS (ESI): m/z 453 [M+H]⁺.

### Step 5. (R)-2-methyl-2-(5-(3-methylmorpholino)-3-(1H-pyrazol-5-yl)isothiazolo[4,5-b]pyridin-7-yl)propanenitrile

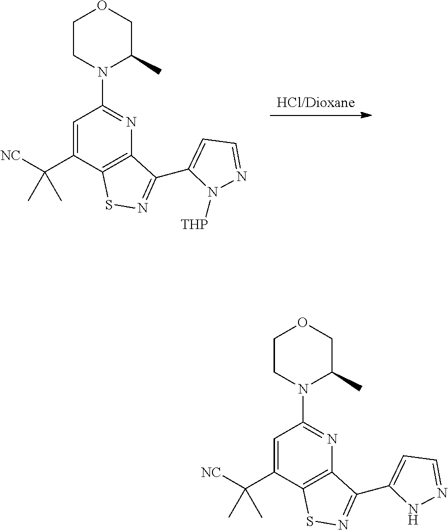

A mixture of 2-methyl-2-{5-[(3S)-3-methylmorpholin-4-yl]-3-[1-(oxan-2-yl)-1H-pyrazol-5-yl]-[1,2]thiazolo[4,5-b]pyridin-7-yl}propanenitrile (80 mg, 0.17 mmol) in HCl solution (4.0 M in dioxane, 2.0 mL) was stirred at room temperature for 1 h. LC-MS showed the reaction was complete. The reaction mixture was concentrated under reduced pressure. The residue was purified by Prep-HPLC (C18, 10-95%, MeOH in H₂O with 0.1% HCOOH) to give the desired product (10 mg, yield: 15%). LC/MS (ESI): m/z 369 [M+H]⁺. ¹H NMR (400 MHz, DMSO) δ 13.51 (d, J=174.9 Hz, 1H), 7.70 (s, 1H), 7.40 (d, J=1.9 Hz, 1H), 7.16 (s, 1H), 4.57 (d, J=4.9 Hz, 1H), 4.12 (d, J=12.3 Hz, 1H), 4.04 (dd, J=11.1, 3.2 Hz, 1H), 3.83 (d, J=11.3 Hz, 1H), 3.71 (dd, J=11.4, 2.8 Hz, 1H), 3.56 (td, J=11.8, 3.0 Hz, 1H), 3.30-3.22 (m, 1H), 1.89 (d, J=1.2 Hz, 6H), 1.25 (d, J=6.7 Hz, 3H).

### Example 54

### Synthesis of (R)-2-methyl-2-(3-(3-methyl-1H-pyrazol-5-yl)-5-(3-methylmorpholino)isothiazolo[4,5-b]pyridin-7-yl)propanenitrile

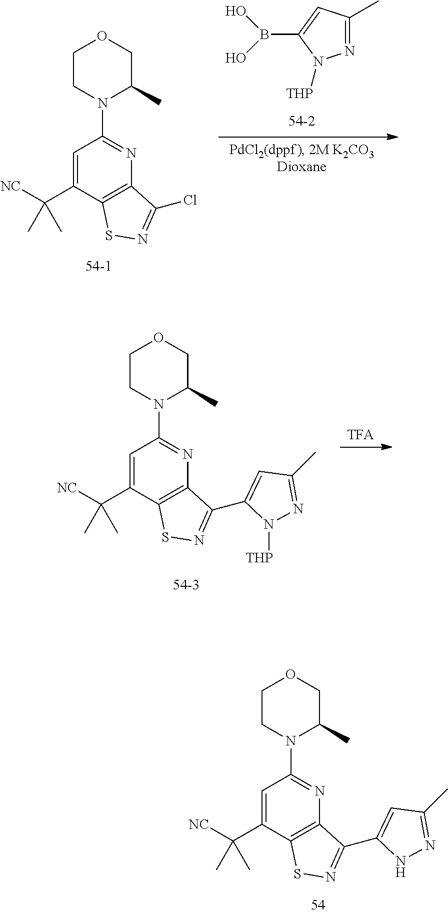

### Step 1. 2-methyl-2-(3-(3-methyl-1-(tetrahydro-2H-pyran-2-yl)-1H-pyrazol-5-yl)-5-((R)-3-methylmorpholino)isothiazolo[4,5-b]pyridin-7-yl)propanenitrile

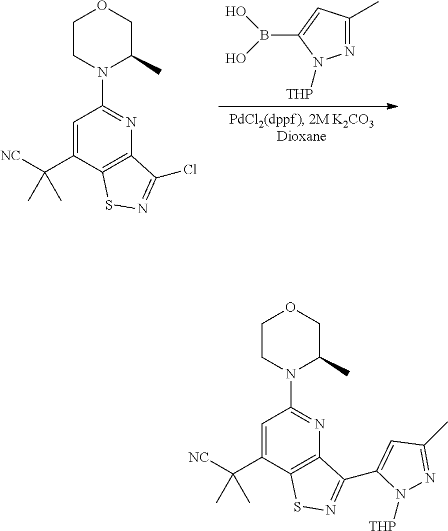

A mixture of 2-{3-chloro-5-[(3R)-3-methylmorpholin-4-yl]-[1,2]thiazolo[4,5-b]pyridin-7-yl}-2-methylpropanenitrile (100 mg, 0.29 mmol), [3-methyl-1-(oxan-2-yl)-1H-pyrazol-5-yl]boronic acid (187 mg, 0.89 mmol), Pd(dppf)Cl₂ (45 mg, 0.06 mmol) and K₂CO₃ (2.0 M in H₂O, 0.45 mL, 0.90 mmol) in Dioxane (6 mL) was stirred at 100° C. for 16 h under N₂ atmosphere. LC-MS showed the reaction was complete. The mixture was diluted with EA (30 mL), then washed with water and brine, dried over anhydrous Na₂SO₄, filtered and concentrated. The residue was purified by flash chromatography on silica gel (DCM:MeOH=20:1, V/V) to afford the desired product (80 mg, yield: 57%). LC/MS (ESI): m/z 467 [M+H]⁺.

### Step 2. (R)-2-methyl-2-(3-(3-methyl-1H-pyrazol-5-yl)-5-(3-methylmorpholino) isothiazolo[4,5-b]pyridin-7-yl)propanenitrile

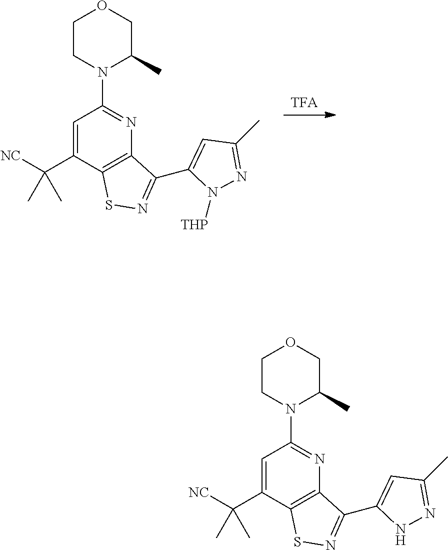

A mixture of 2-methyl-2-{3-[3-methyl-1-(oxan-2-yl)-1H-pyrazol-5-yl]-5-[(3S)-3-methylmorpholin-4-yl]-[1,2]thiazolo[4,5-b]pyridin-7-yl}propanenitrile (100 mg, 0.21 mmol) in TFA (4.0 mL) was stirred at 25° C. for 2 h. LC-MS showed the reaction was complete. The reaction mixture was concentrated under reduced pressure. The residue was purified by Prep-HPLC (C18, 10-95%, MeOH in H₂O with 0.1% HCOOH) to give the desired product (20 mg, yield: 16%). LC/MS (ESI): m/z 383 [M+H]⁺. ¹H NMR (400 MHz, DMSO) δ 13.26-12.95 (m, 1H), 7.13 (t, J=13.3 Hz, 2H), 4.55 (s, 1H), 4.08 (dd, J=31.5, 11.5 Hz, 2H), 3.83 (d, J=11.4 Hz, 1H), 3.71 (d, J=9.1 Hz, 1H), 3.57 (t, J=10.5 Hz, 1H), 3.28 (s, 1H), 2.32 (d, J=21.5 Hz, 3H), 1.89 (d, J=1.2 Hz, 6H), 1.25 (d, J=6.6 Hz, 3H).

### Example 55

### Synthesis of (R)-3-methyl-4-(7-(2-(methylsulfonyl)propan-2-yl)-3-(1H-pyrazol-5-yl)isothiazolo[4,5-b]pyridin-5-yl)morpholine

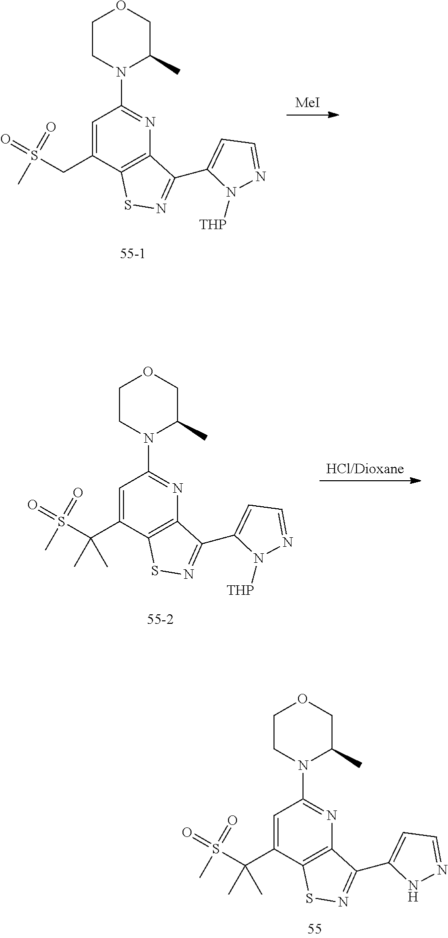

### Step 1. (3R)-3-methyl-4-(7-(2-(methylsulfonyl)propan-2-yl)-3-(1-(tetrahydro-2H-pyran-2-yl)-1H-pyrazol-5-yl)isothiazolo[4,5-b]pyridin-5-yl)morpholine

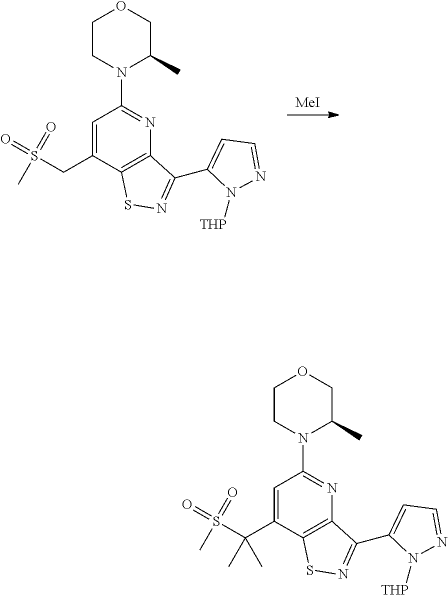

To a solution of (3R)-3-methyl-4-(7-((methylsulfonyl)methyl)-3-(1-(tetrahydro-2H-pyran-2-yl)-1H-pyrazol-5-yl)isothiazolo[4,5-b]pyridin-5-yl)morpholine (30 mg, 0.06 mmol) and t-BuONa (18 mg, 0.19 mmol) in THF (6 mL) was added MeI (27 mg, 0.19 mmol). The mixture was stirred at room temperature for 16 h. LC-MS showed the reaction was complete. The mixture was diluted with EA (40 mL), then washed with water and brine, dried over anhydrous Na₂SO₄, filtered and concentrated. The residue was purified by column chromatography on silica gel (PE:EA=5:1, V/V) to afford the desired product (27 mg, yield: 85%). LC/MS (ESI): m/z 506 [M+H]⁺.

### Step 2. (R)-3-methyl-4-(7-(2-(methylsulfonyl)propan-2-yl)-3-(1H-pyrazol-5-yl)isothiazolo[4,5-b]pyridin-5-yl)morpholine

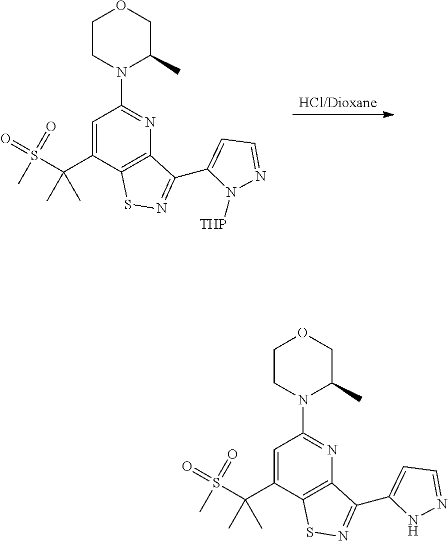

To a mixture of (3R)-3-methyl-4-(7-(2-(methylsulfonyl)propan-2-yl)-3-(1-(tetrahydro-2H-pyran-2-yl)-1H-pyrazol-5-yl)isothiazolo[4,5-b]pyridin-5-yl)morpholine (27 mg, 0.05 mmol) in DCM (0.5 mL) was added HCl solution (4M in dioxane, 1.5 mL). The mixture was stirred at room temperature for 1 h. LC-MS showed the reaction was complete. The reaction mixture was concentrated under vacuo. The residue was purified by Prep-HPLC (C₁₈, 10-95%, MeOH in H₂O with 0.1% HCOOH) to give the desired product (5.8 mg, yield: 25.8%). LC/MS (ESI): m/z 422 [M+H]⁺. ¹H NMR (400 MHz, DMSO) δ 7.75 (s, 1H), 7.37 (d, J=1.9 Hz, 1H), 7.27 (s, 1H), 4.59-4.51 (m, 1H), 4.16-4.09 (m, 1H), 4.05 (dd, J=11.5, 3.4 Hz, 1H), 3.83 (d, J=11.2 Hz, 1H), 3.74 (dd, J=11.4, 2.8 Hz, 1H), 3.62-3.55 (m, 1H), 3.27-3.25 (m, 1H), 2.92 (s, 3H), 1.98 (d, J=4.0 Hz, 6H), 1.23 (d, J=6.7 Hz, 3H).

### Example 56

### Synthesis of (R)-3-methyl-4-(3-(3-methyl-1H-pyrazol-5-yl)-7-(2-(methylsulfonyl)propan-2-yl)isothiazolo[4,5-b]pyridin-5-yl)morpholine

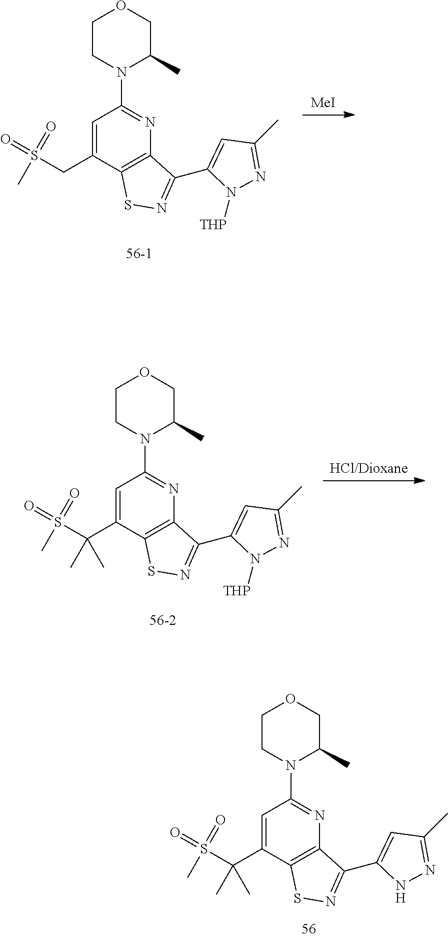

Step 1. (3R)-3-methyl-4-(3-(3-methyl-1-(tetrahydro-2H-pyran-2-yl)-1H-pyrazol-5-yl)-7-(2-(methylsulfonyl)propan-2-yl)isothiazolo[4,5-b]pyridin-5-yl)morpholine

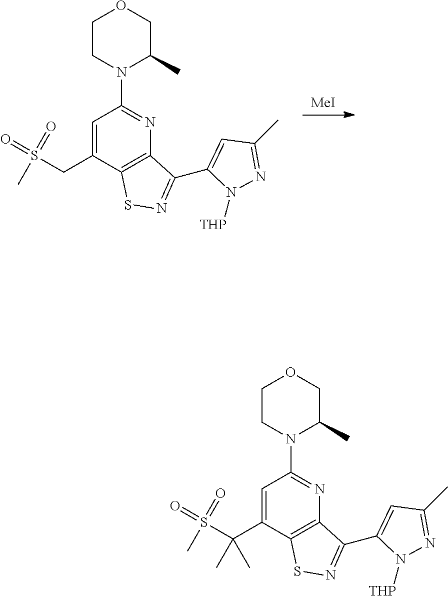

To a solution of (3R)-3-methyl-4-(3-(3-methyl-1-(tetrahydro-2H-pyran-2-yl)-1H-pyrazol-5-yl)-7-((methylsulfonyl)methyl)isothiazolo[4,5-b]pyridin-5-yl)morpholine (30 mg, 0.06 mmol) and t-BuONa (18 mg, 0.18 mmol) in THF (3 mL) was added MeI (26 mg, 0.18 mmol). The mixture was stirred at room temperature for 16 h. LC-MS showed the reaction was complete. LC-MS showed the reaction was complete. The mixture was diluted with EA (40 mL), then washed with water and brine, dried over anhydrous Na₂SO₄, filtered and concentrated. The residue was purified by column chromatography on silica gel (PE:EA=3:1, V/V) to afford the desired product (24 mg, yield: 76%). LC/MS (ESI): m/z 520 [M+H]⁺.

### Step 2. (R)-3-methyl-4-(3-(3-methyl-1H-pyrazol-5-yl)-7-(2-(methylsulfonyl)propan-2-yl)isothiazolo[4,5-b]pyridin-5-yl)morpholine

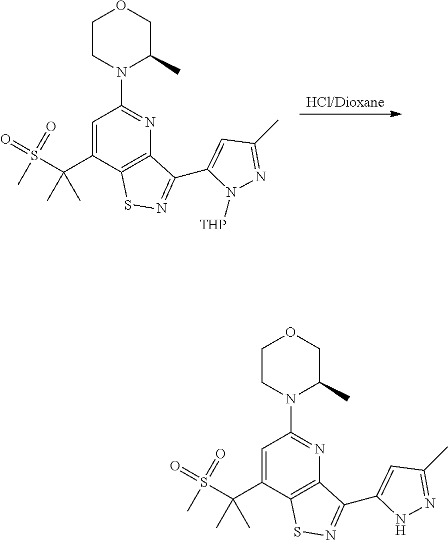

To a mixture of (3R)-3-methyl-4-(3-(3-methyl-1-(tetrahydro-2H-pyran-2-yl)-1H-pyrazol-5-yl)-7-(2-(methylsulfonyl)propan-2-yl)isothiazolo[4,5-b]pyridin-5-yl) morpholine (24 mg, 0.05 mmol) in DCM (1.0 mL) was added HCl solution (4M in dioxane, 1.0 mL). The mixture was stirred at room temperature for 1 h. LC-MS showed the reaction was complete. The mixture was concentrated in vacuo to dryness. The residue was purified by Prep-HPLC (C₁₈, 10-95%, MeCN in H₂O with 0.1% HCOOH) to give the desired product (6.4 mg, yield: 32%). LC/MS (ESI): m/z 436 [M+H]⁺. ¹H NMR (400 MHz, DMSO) δ 13.11 (s, 1H), 7.26 (s, 1H), 7.10 (s, 1H), 4.59-4.47 (m, 1H), 4.12 (dd, J=12.8, 1.6 Hz, 1H), 4.05 (dd, J=11.4, 3.3 Hz, 1H), 3.83 (d, J=11.2 Hz, 1H), 3.74 (dd, J=11.3, 2.8 Hz, 1H), 3.59 (td, J=11.8, 2.8 Hz, 1H), 3.28-3.24 (m, 1H), 2.91 (s, 3H), 2.31 (s, 3H), 1.98 (d, J=4.2 Hz, 6H), 1.23 (d, J=6.6 Hz, 3H).

### Example 57

### Synthesis of (R)-1-(5-(3-methylmorpholino)-3-(1H-pyrazol-5-yl)isothiazolo[4,5-b]pyridin-7-yl)cyclopentane-1-carbonitrile

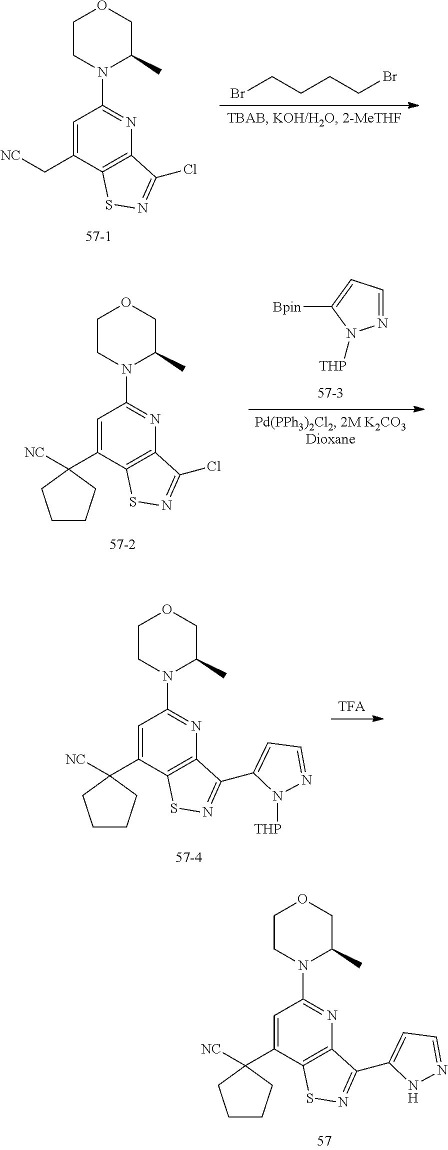

### Step 1. (R)-1-(3-chloro-5-(3-methylmorpholino)isothiazolo[4,5-b]pyridin-7-yl)cyclopentane-1-carbonitrile

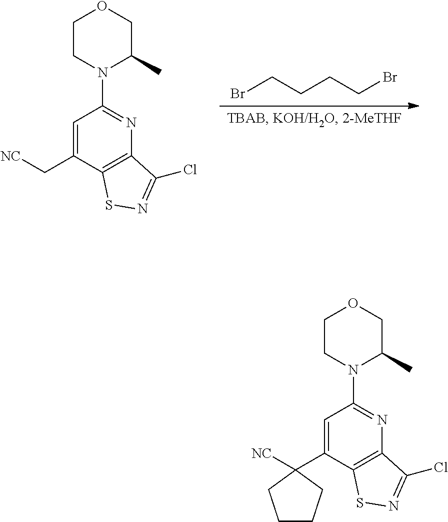

A mixture of 2-{3-chloro-5-[(3R)-3-methylmorpholin-4-yl]-[1,2]thiazolo[4,5-b] pyridin-7-yl}acetonitrile (158 mg, 0.51 mmol), 1,4-dibromobutane (443 mg, 2.05 mmol), TBAB (33 mg, 0.10 mmol) and KOH (10.0 M in H₂O, 1.0 mL, 10.0 mmol) in 2-Methyltetrahydrofuran (10 mL) was stirred at 80° C. for 3 h. LC-MS showed the reaction was complete. The reaction mixture was diluted with EA (50 mL), then washed with water and brine, dried over anhydrous Na₂SO₄, filtered and concentrated. The residue was purified by column chromatography on silica gel (DCM:MOH=40:1, V/V) to afford the desired product (125 mg, yield: 67%). LC/MS (ESI): m/z 363 [M+H]⁺.

### Step 2. 1-(5-((R)-3-methylmorpholino)-3-(1-(tetrahydro-2H-pyran-2-yl)-1H-pyrazol-5-yl)isothiazolo[4,5-b]pyridin-7-yl)cyclopentane-1-carbonitrile

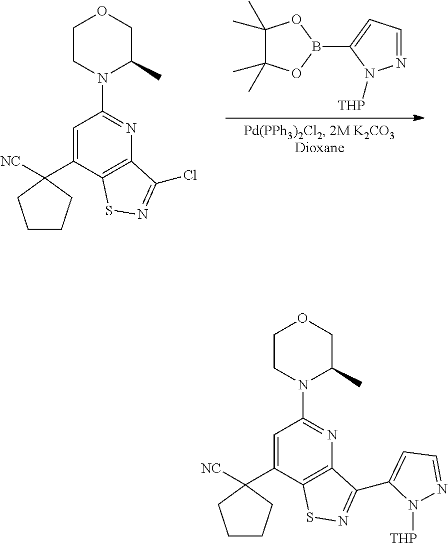

A mixture of 1-{3-chloro-5-[(3R)-3-methylmorpholin-4-yl]-[1,2]thiazolo[4,5-b]pyridin-7-yl}cyclopentane-1-carbonitrile (113 mg, 0.31 mmol), 1-(oxan-2-yl)-5-(tetramethyl-1,3,2-dioxaborolan-2-yl)-1H-pyrazole (217 mg, 0.78 mmol), Pd(dppf)Cl₂ (45 mg, 0.06 mmol) and K₂CO₃ (2.0 M in H₂O, 0.46 mL, 0.92 mmol) in DME (5 mL) was stirred at 100° C. for 16 h under N₂ atmosphere. LC-MS showed the reaction was complete. The reaction mixture was diluted with H₂O (20 mL), then extracted with EA (50 mL×3). The combined organic layer was washed with brine, dried over anhydrous Na₂SO₄, filtered and concentrated. The residue was purified by column chromatography on silica gel (DCM:MeOH=40:1, V/V) to give the desired product (80 mg, yield: 53%). LC/MS (ESI): m/z 479 [M+H]⁺.

### Step 3. (R)-1-(5-(3-methylmorpholino)-3-(1H-pyrazol-5-yl)isothiazolo[4,5-b]pyridin-7-yl)cyclopentane-1-carbonitrile

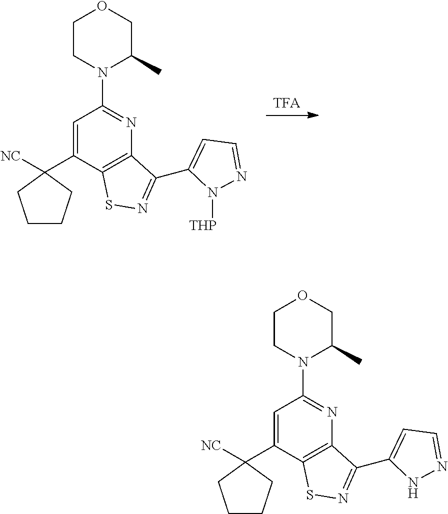

A mixture of 1-{5-[(3S)-3-methylmorpholin-4-yl]-3-[1-(oxan-2-yl)-1H-pyrazol-5-yl]-[1,2]thiazolo[4,5-b]pyridin-7-yl}cyclopentane-1-carbonitrile (130 mg, 0.27 mmol) in TFA (6.0 mL) was stirred at room temperature for 1 h. LC-MS showed the reaction was complete. The reaction mixture was concentrated under reduced pressure. The residue was purified by Prep-HPLC (C18, 10-95%, MeOH in H₂O with 0.1% HCOOH) to give the desired product (20 mg, yield: 18%). LC/MS (ESI): m/z 395 [M+H]⁺. ¹H NMR (400 MHz, DMSO) δ 13.51 (d, J=175.6 Hz, 1H), 7.80 (d, J=90.8 Hz, 1H), 7.40 (s, 1H), 7.17 (d, J=13.3 Hz, 1H), 4.58 (s, 1H), 4.21-3.97 (m, 2H), 3.83 (d, J=11.4 Hz, 1H), 3.71 (dd, J=11.4, 2.7 Hz, 1H), 3.56 (t, J=10.5 Hz, 1H), 3.28 (s, 1H), 2.65-2.56 (m, 2H), 2.40-2.31 (m, 2H), 1.97 (t, J=6.1 Hz, 4H), 1.25 (d, J=6.5 Hz, 3H).

### Example 58

### Synthesis of (R)-1-(5-(3-methylmorpholino)-3-(1H-pyrazol-5-yl)isothiazolo[4,5-b]pyridin-7-yl)cyclohexane-1-carbonitrile

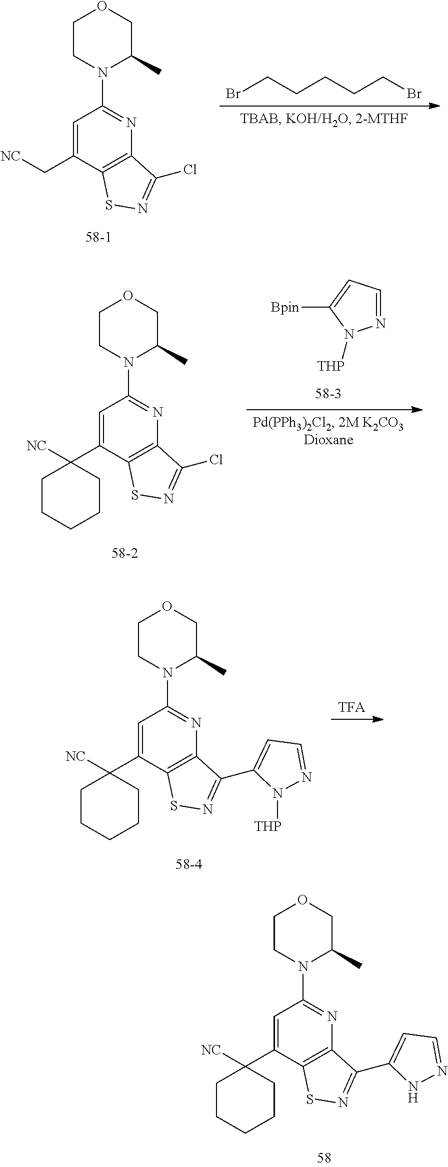

### Step 1. (R)-1-(3-chloro-5-(3-methylmorpholino)isothiazolo[4,5-b]pyridin-7-yl) cyclohexane-1-carbonitrile

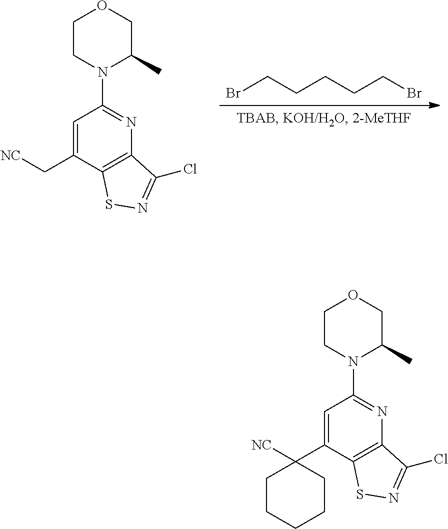

A mixture of 2-{3-chloro-5-[(3R)-3-methylmorpholin-4-yl]-[1,2]thiazolo[4,5-b]pyridin-7-yl}acetonitrile (158 mg, 0.51 mmol), 1,5-dibromopentane (470 mg, 2.05 mmol), TBAB (33 mg, 0.10 mmol) and KOH (10.0 M in H₂O, 1.0 mL, 10.0 mmol) in 2-Methyltetrahydrofuran (10 mL) was stirred at 80° C. for 3 h. LC-MS showed the reaction was complete. The reaction mixture was diluted with EA (200 mL), then washed with water and brine, dried over anhydrous Na₂SO₄, filtered and concentrated. The residue was purified by column chromatography on silica gel (DCM:MeOH=40:1, V/V) to afford the desired product (161 mg, yield: 83%). LC/MS (ESI): m/z 377 [M+H]⁺.

### Step 2. 1-(5-((R)-3-methylmorpholino)-3-(1-(tetrahydro-2H-pyran-2-yl)-1H-pyrazol-5-yl)isothiazolo[4,5-b]pyridin-7-yl)cyclohexane-1-carbonitrile

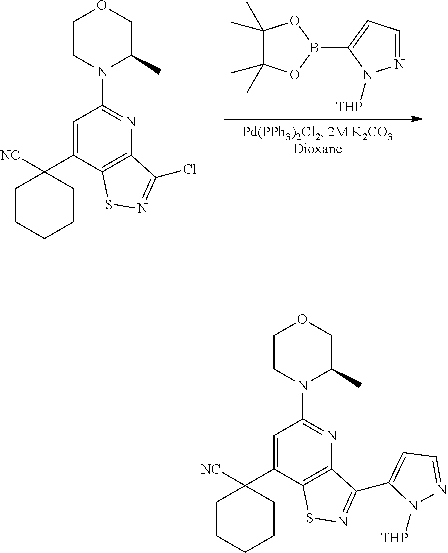

A mixture of 1-{3-chloro-5-[(3R)-3-methylmorpholin-4-yl]-[1,2]thiazolo[4,5-b]pyridin-7-yl}cyclohexane-1-carbonitrile (145 mg, 0.38 mmol), 1-(oxan-2-yl)-5-(tetramethyl-1,3,2-dioxaborolan-2-yl)-1H-pyrazole (168 mg, 0.96 mmol), Pd(dppf)Cl₂ (56 mg, 0.07 mmol) and K₂CO₃ (2.0 M in H₂O, 0.58 mL, 1.16 mmol) in DME (5 mL) was stirred at 100° C. for 16 h under N₂ atmosphere. LC-MS showed the reaction was complete. The reaction mixture was diluted with H₂O (20 mL), then extracted with EA (50 mL×3). The combined organic layer was washed with brine, dried over anhydrous Na₂SO₄, filtered and concentrated. The residue was purified by column chromatography on silica gel (DCM:MeOH=40:1, V/V) to give the desired product (100 mg, yield: 52%). LC/MS (ESI): m/z 493 [M+H]⁺.

### Step 3. (R)-1-(5-(3-methylmorpholino)-3-(1H-pyrazol-5-yl)isothiazolo[4,5-b]pyridin-7-yl)cyclohexane-1-carbonitrile

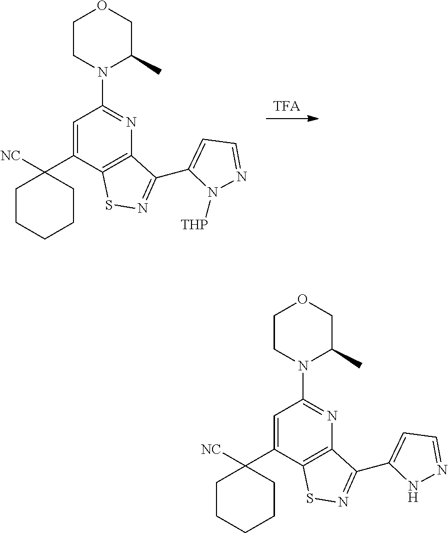

A mixture of 1-{5-[(3S)-3-methylmorpholin-4-yl]-3-[1-(oxan-2-yl)-1H-pyrazol-5-yl]-[1,2]thiazolo[4,5-b]pyridin-7-yl}cyclohexane-1-carbonitrile (100 mg, 0.20 mmol) in TFA (6.0 mL) was stirred at room temperature for 2 h. LC-MS showed the reaction was complete. The reaction mixture was concentrated under reduced pressure. The residue was purified by Prep-HPLC (C18, 10-95%, MeOH in H₂O with 0.1% HCOOH) to give the desired product (20 mg, yield: 24%). LC/MS (ESI): m/z 409 [M+H]⁺. ¹H NMR (400 MHz, DMSO) δ 13.51 (d, J=173.9 Hz, 1H), 7.80 (d, J=87.1 Hz, 1H), 7.39 (d, J=1.6 Hz, 1H), 7.20 (s, 1H), 4.57 (s, 1H), 4.12 (d, J=12.6 Hz, 1H), 4.04 (d, J=8.5 Hz, 1H), 3.83 (d, J=11.3 Hz, 1H), 3.71 (dd, J=11.3, 2.6 Hz, 1H), 3.56 (dd, J=11.7, 9.1 Hz, 1H), 3.27 (d, J=12.7 Hz, 1H), 2.35 (d, J=13.0 Hz, 2H), 2.07 (dd, J=17.1, 8.9 Hz, 2H), 1.93 (d, J=13.9 Hz, 2H), 1.75 (dt, J=39.1, 13.2 Hz, 3H), 1.42-1.33 (m, 1H), 1.25 (d, J=6.6 Hz, 3H).

### Example 59

### Synthesis of 1-{2-[(3R)-3-methylmorpholin-4-yl]-7-(1H-pyrazol-5-yl)imidazo[1,5-b]pyridazin-4-yl}cyclopentane-1-carbonitrile

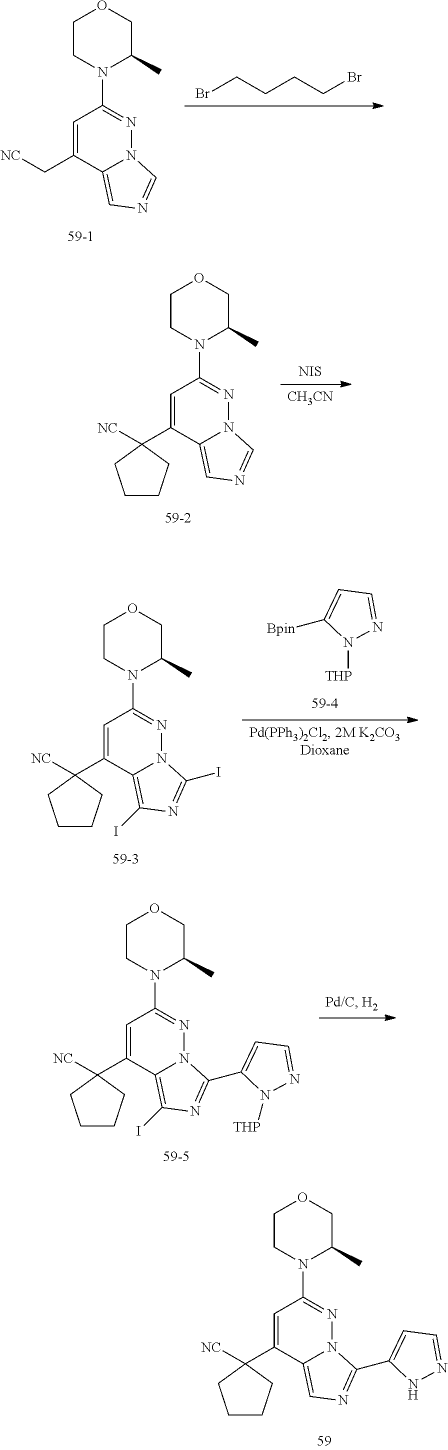

### Step 1. 1-{2-[(3R)-3-methylmorpholin-4-yl]imidazo[1,5-b]pyridazin-4-yl}cyclopentane-1-carbonitrile

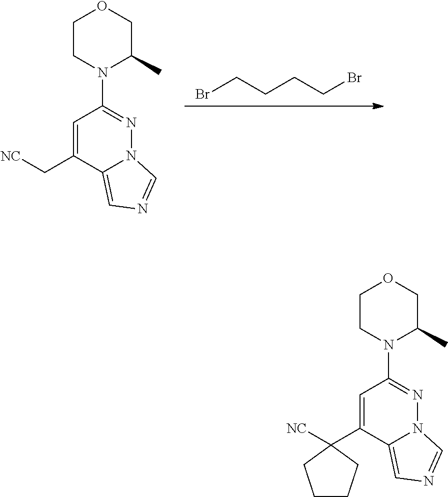 [SMILES: C[C@@H]1COCCN1c1cc(CC#N)c2cncn2n1 ; BrCCCCBr ; C[C@@H]1COCCN1c1cc(C2(C#N)CCCC2)c2cncn2n1]

To a solution of 2-{2-[(3R)-3-methylmorpholin-4-yl]imidazo[1,5-b]pyridazin-4-yl}acetonitrile (250 mg, 0.97 mmol) in 2-Methyltetrahydrofuran (15 mL) were added 1,4-dibromobutane (1.16 mL, 9.72 mmol), TBAB (42 mg, 0.19 mmol) and KOH (10M in H₂O, 6.8 mL, 68.01 mmol). The reaction was stirred at 70° C. overnight. LC-MS showed the reaction was complete. The mixture was diluted with EA (40 mL), then washed with water and brine, dried over anhydrous Na₂SO₄, filtered and concentrated. The residue was purified by column chromatography on silica gel (PE:EA=5:1, V/V) to afford the desired product (250 mg, yield: 82.63%). LC/MS (ESI): m/z 312 [M+H]⁺.

### Step 2.1-{5,7-diiodo-2-[(3R)-3-methylmorpholin-4-yl]imidazo[1,5-b]pyridazin-4-yl}cyclopentane-1-carbonitrile

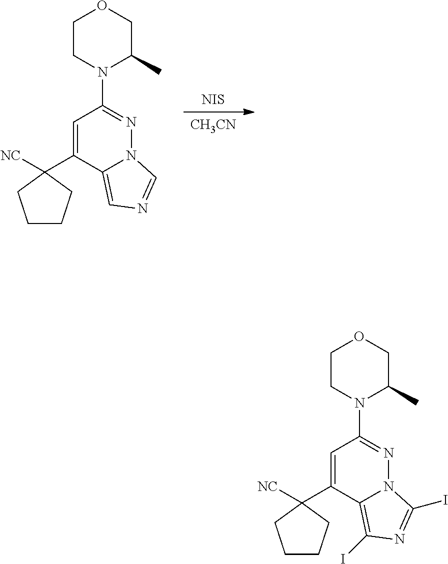

To a solution of 1-{2-[(3R)-3-methylmorpholin-4-yl]imidazo[1,5-b]pyridazin-4-yl}cyclopentane-1-carbonitrile (250 mg, 0.80 mmol) in CH₃CN (15 mL) was added NIS (180.6 mg, 0.80 mmol). The mixture was stirred at 80° C. overnight. LC-MS showed the reaction was complete. The mixture was diluted with EA (40 mL), then washed with saturated Na₂S₂O₃ aqueous solution and brine, dried over anhydrous Na₂SO₄, filtered and concentrated. The residue was purified by column chromatography on silica gel (PE:EA=5:1, V/V) to afford the desired product (150 mg, yield: 33.17%). LC/MS (ESI): m/z 564 [M+H]⁺.

### Step 3. 1-{5-iodo-2-[(3R)-3-methylmorpholin-4-yl]-7-[1-(oxan-2-yl)-1H-pyrazol-5-yl]imidazo[1,5-b]pyridazin-4-yl}cyclopentane-1-carbonitrile

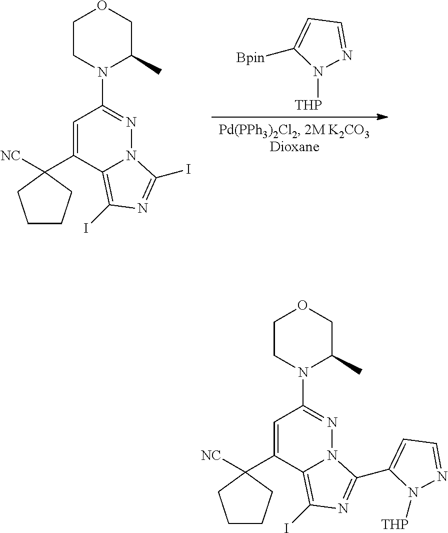

To a solution of 1-{5,7-diiodo-2-[(3R)-3-methylmorpholin-4-yl]imidazo[1,5-b]pyridazin-4-yl}cyclopentane-1-carbonitrile (130 mg, 0.23 mmol) in dioxane (8 mL) were added 1-(oxan-2-yl)-5-(tetramethyl-1,3,2-dioxaborolan-2-yl)-1H-pyrazole (128 mg, 0.46 mmol), Pd(PPh₃)₂Cl₂ (33 mg, 0.05 mmol) and K₂CO₃ (95.71 mg, 0.69 mmol). The reaction was stirred at 80° C. overnight under nitrogen atmosphere. LC-MS showed the reaction was complete. The mixture was diluted with EA (40 mL), then washed with water and brine, dried over anhydrous Na₂SO₄, filtered and concentrated. The residue was purified by column chromatography on silica gel (PE:EA=5:1, V/V) to afford the desired product (66 mg, yield: 48.67%). LC/MS (ESI): m/z 588 [M+H]⁺.

### Step 4. 1-{2-[(3R)-3-methylmorpholin-4-yl]-7-(1H-pyrazol-5-yl)imidazo[1,5-b]pyridazine-4-yl}cyclopentane-1-carbonitrile

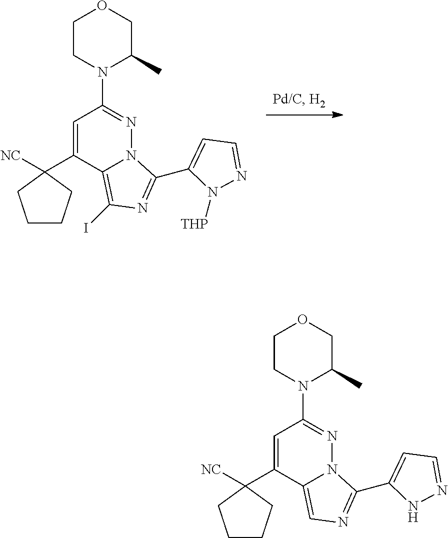

To a solution of 1-{5-iodo-2-[(3R)-3-methylmorpholin-4-yl]-7-[1-(oxan-2-yl)-1H-pyrazol-5-yl]imidazo[1,5-b]pyridazin-4-yl}cyclopentane-1-carbonitrile (66 mg, 0.11 mmol) in MeOH (3 ml) was added Pd/C (10%, 35.87 mg). The mixture was stirred at room temperature overnight under H₂ atmosphere. LC-MS showed the reaction was complete. The mixture was filtered, then concentrated in vacuo. The residue was purified by Prep-HPLC (C₁₈, 10-95%, MeOH in H₂O with 0.1% HCOOH) to give the desired product (1 mg, yield: 2.36%). LC/MS (ESI): m/z 378 [M+H]⁺. ¹H NMR (400 MHz, DMSO) δ 8.34 (s, 1H), 8.08 (s, 1H), 7.28 (d, J=2.2 Hz, 1H), 7.06 (s, 1H), 4.48 (d, J=4.5 Hz, 1H), 4.19-3.91 (m, 2H), 3.81 (d, J=11.7 Hz, 1H), 3.70 (d, J=9.1 Hz, 1H), 3.56 (dd, J=11.8, 9.2 Hz, 1H), 3.36 (dd, J=17.5, 8.1 Hz, 1H), 2.72-2.59 (m, 2H), 2.39-2.25 (m, 2H), 1.94 (s, 4H), 1.30 (d, J=6.7 Hz, 3H).

### Example 60

### Synthesis of (R)-1-(2-(3-methylmorpholino)-7-(1H-pyrazol-5-yl)imidazo[1,5-b]pyridazin-4-yl)cyclohexane-1-carbonitrile

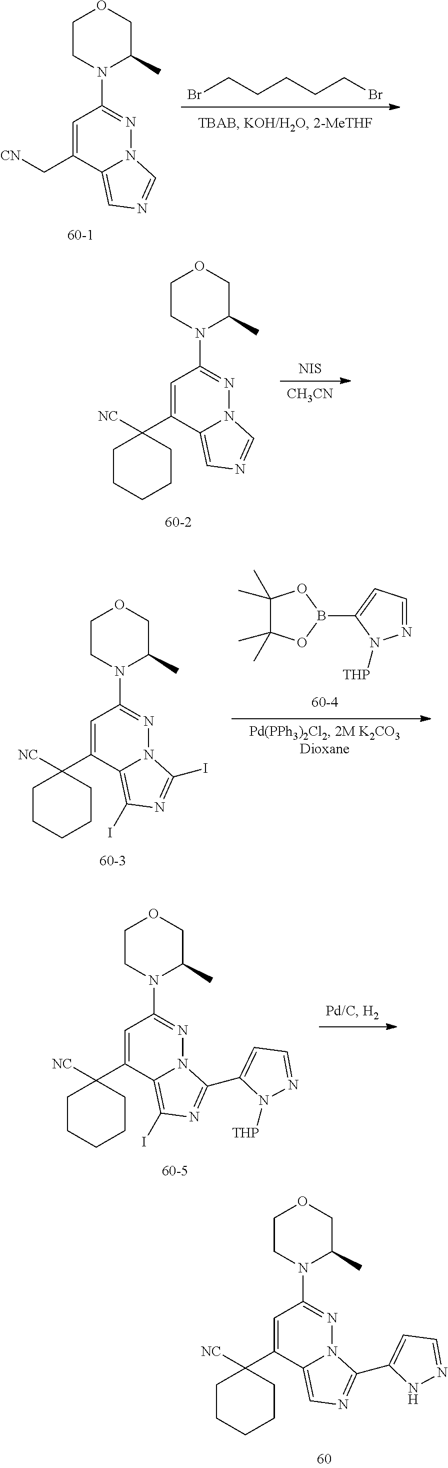

### Step 1. (R)-1-(2-(3-methylmorpholino)imidazo[1,5-b]pyridazin-4-yl)cyclohexane-1-carbonitrile

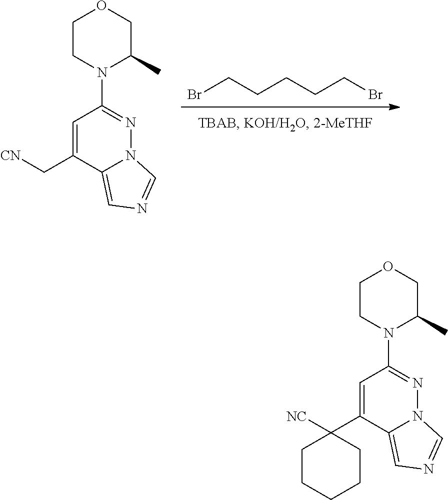

A mixture of 2-{2-[(3R)-3-methylmorpholin-4-yl]imidazo[1,5-b]pyridazin-4-yl} acetonitrile (500 mg, 1.94 mmol), 1,2-dibromoethane (1.78 g, 7.77 mmol), TBAB (125 mg, 0.38 mmol) and KOH (10.0 M in H₂O, 3.8 mL, 38.8 mmol) in 2-Methyltetrahydrofuran (40 mL) was stirred at 80° C. for 4 h. LC-MS showed the reaction was complete. The reaction mixture was diluted with EA (200 mL), then washed with water and brine, dried over anhydrous Na₂SO₄, filtered and concentrated. The residue was purified by column chromatography on silica gel (DCM:MeOH=15:1, V/V) to afford the desired product (430 mg, yield: 68%). LC/MS (ESI): m/z 326 [M+H]⁺.

### Step 2. (R)-1-(5,7-diiodo-2-(3-methylmorpholino)imidazo[1,5-b]pyridazin-4-yl)cyclohexane-1-carbonitrile

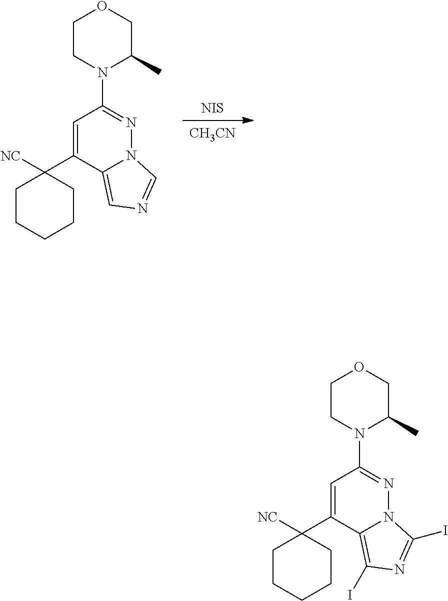

A mixture of 1-{2-[(3R)-3-methylmorpholin-4-yl]imidazo[1,5-b]pyridazin-4-yl} cyclohexane-1-carbonitrile (430 mg, 1.32 mmol) and NIS (1.19 g, 5.28 mmol) in MeCN (10 mL) was stirred at 80° C. for 4 h. LC-MS showed the reaction was complete. The reaction mixture was diluted with DCM (40 mL), then washed with water and brine, dried over anhydrous Na₂SO₄, filtered and concentrated. The residue was purified by column chromatography on silica gel (PE:EA=2:1, V/V) to afford the desired product (356 mg, yield: 46%). LC/MS (ESI): m/z 578 [M+H]⁺.

### Step 3. 1-(5-iodo-2-((R)-3-methylmorpholino)-7-(1-(tetrahydro-2H-pyran-2-yl)-1H-pyrazol-5-yl)imidazo[1,5-b]pyridazin-4-yl)cyclohexane-1-carbonitrile

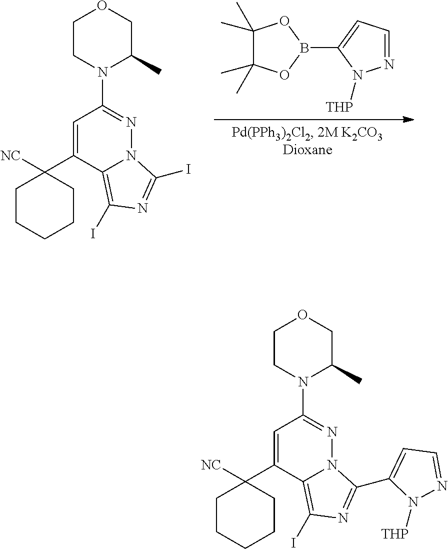

A mixture of 1-{5,7-diiodo-2-[(3R)-3-methylmorpholin-4-yl]imidazo[1,5-b]pyridazin-4-yl}cyclohexane-1-carbonitrile (195 mg, 0.34 mmol), 1-(oxan-2-yl)-5-(tetramethyl-1,3,2-dioxaborolan-2-yl)-1H-pyrazole (122 mg, 0.44 mmol), PdCl₂(PPh₃)₂ (25 mg, 0.03 mmol) and K₂CO₃ (2.0 M in H₂O, 0.34 mL, 0.68 mmol) in DME (20 mL) was stirred at 100° C. for 16 h under N₂ atmosphere. LC-MS showed the reaction was complete. The reaction mixture was diluted with H₂O (40 mL), then extracted with EA (50 mL×3). The combined organic layer was washed with brine, dried over anhydrous Na₂SO₄, filtered and concentrated. The residue was purified by column chromatography on silica gel (DCM:MeOH=20:1, V/V) to give the desired product (50 mg, yield: 24%). LC/MS (ESI): m/z 602 [M+H]⁺.

### Step 4. (R)-1-(2-(3-methylmorpholino)-7-(1H-pyrazol-5-yl)imidazo[1,5-b]pyridazin-4-yl)cyclohexane-1-carbonitrile

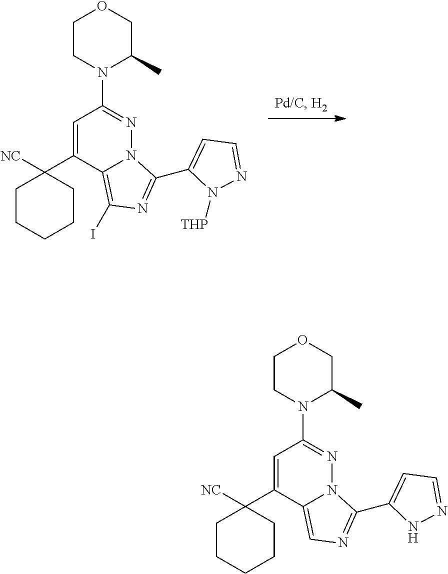

A mixture of 1-{5-iodo-2-[(3R)-3-methylmorpholin-4-yl]-7-[1-(oxan-2-yl)-1H-pyrazol-5-yl]imidazo[1,5-b]pyridazin-4-yl}cyclohexane-1-carbonitrile (50 mg, 0.08 mmol) and Pd/C (10%, 20 mg) in MeOH (3 mL) was stirred at room temperature for 16 h under H₂ atmosphere. LC-MS showed the reaction was complete. The reaction mixture was filtered, then concentrated under reduced pressure to dryness. The residue was purified by Prep-HPLC (C18, 10-95%, MeOH in H₂O with 0.1% HCOOH) to give the desired product (10 mg, yield: 30%). LC/MS(ESI): m/z 392 [M+H]⁺. ¹H NMR (400 MHz, DMSO) δ 13.34 (d, J=164.8 Hz, 1H), 7.84 (d, J=28.4 Hz, 1H), 7.68 (d, J=34.9 Hz, 1H), 7.10 (s, 1H), 6.76 (d, J=22.2 Hz, 1H), 4.37 (s, 1H), 4.01 (d, J=10.2 Hz, 1H), 3.93-3.67 (m, 3H), 3.56 (t, J=10.6 Hz, 1H), 3.28 (d, J=13.6 Hz, 1H), 2.35 (d, J=13.7 Hz, 2H), 2.03 (dd, J=20.3, 14.4 Hz, 2H), 1.90 (d, J=13.9 Hz, 2H), 1.83-1.64 (m, 3H), 1.45-1.32 (m, 1H), 1.25 (d, J=6.4 Hz, 3H).

### Example 61

### Synthesis of (3R)-4-[5-chloro-4-(1-methyl-1H-pyrazol-5-yl)-7-(1H-pyrazol-5-yl) imidazo[1,5-b]pyridazin-2-yl]-3-methylmorpholine

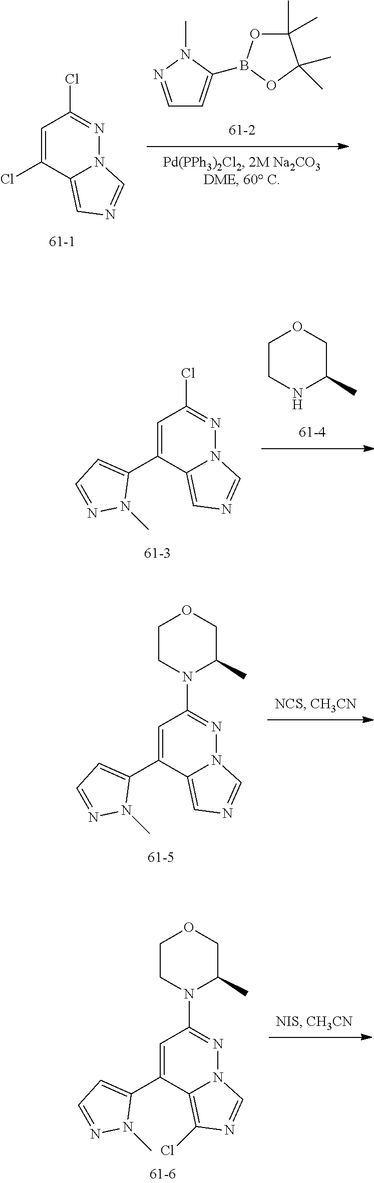

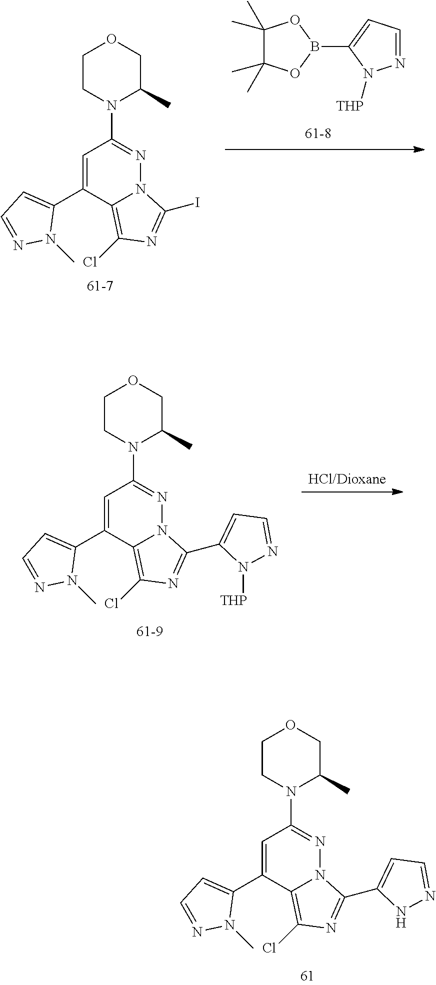

### Step 1. 5-{2-chloroimidazo[1,5-b]pyridazin-4-yl}-1-methyl-1H-pyrazole

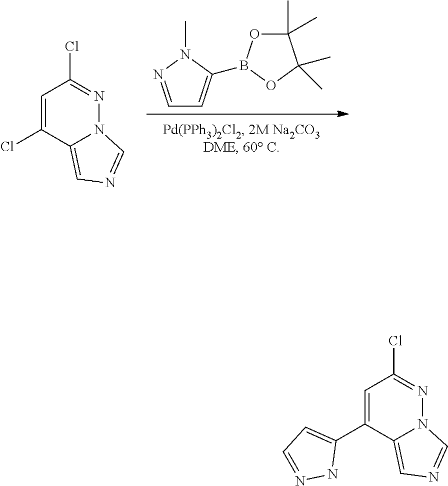

To a solution of 2,4-dichloroimidazo[1,5-b]pyridazine (3 g, 15.96 mmol) and 1-methyl-5-(tetramethyl-1,3,2-dioxaborolan-2-yl)-1H-pyrazole (4.32 g, 20.74 mmol) in DME (90 mL) were added Pd(PPh₃)₂Cl₂ (1.12 g, 1.60 mmol) and Na₂CO₃ (2M in H₂O, 16.0 mL, 31.91 mmol). The reaction was stirred at 60° C. overnight under nitrogen atmosphere. LC-MS showed the reaction was complete. The reaction mixture was diluted with DCM (60 mL), then washed with water and brine, dried over anhydrous Na₂SO₄, filtered and concentrated. The residue was purified by column chromatography on silica gel (PE:EA=3:1, V/V) to afford the desired product (2.25 g, yield: 60%). LC/MS (ESI): m/z 234 [M+H]⁺.

### Step 2. (3R)-3-methyl-4-[4-(1-methyl-1H-pyrazol-5-yl)imidazo[1,5-b]pyridazin-2-yl]morpholine

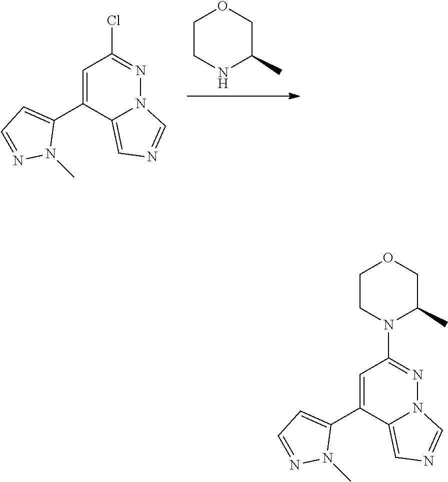

To a solution of 5-{2-chloroimidazo[1,5-b]pyridazin-4-yl}-1-methyl-1H-pyrazole (2.25 g, 9.63 mmol) in sulfolane (50 mL) were added (3R)-3-methylmorpholine (2.92 g, 28.89 mmol) and KF (1.68 g, 28.89 mmol). The reaction was stirred at 180° C. for 8 h. LC-MS showed the reaction was complete. The reaction mixture was diluted with DCM (60 mL), then washed with water and brine, dried over anhydrous Na₂SO₄, filtered and concentrated. The residue was purified by column chromatography on silica gel (PE:EA=1:1, V/V) to afford the desired product (710 mg, yield: 25%). LC/MS (ESI): m/z 299 [M+H]⁺.

### Step 3. (3R)-4-[5-chloro-4-(1-methyl-1H-pyrazol-5-yl)imidazo[1,5-b]pyridazin-2-yl]-3-methylmorpholine

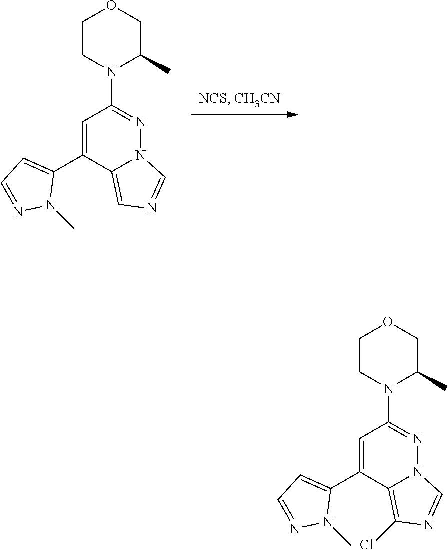

To a solution of (3R)-3-methyl-4-[4-(1-methyl-1H-pyrazol-5-yl)imidazo[1,5-b] pyridazin-2-yl]morpholine (400 mg, 1.34 mmol) in CH₃CN (20 mL) were added NCS (179 mg, 1.34 mmol). The reaction was stirred at 80° C. for 4 h. LC-MS showed the reaction was complete. The reaction mixture was diluted with DCM (60 mL), then washed with water and brine, dried over anhydrous Na₂SO₄, filtered and concentrated. The residue was purified by column chromatography on silica gel (DCM:MeOH=30:1, V/V) to afford the desired product (190 mg, yield: 42%). LC/MS (ESI): m/z 333 [M+H]⁺.

### Step 4. (3R)-4-[5-chloro-7-iodo-4-(1-methyl-1H-pyrazol-5-yl)imidazo[1,5-b]pyridazin-2-yl]-3-methylmorpholine

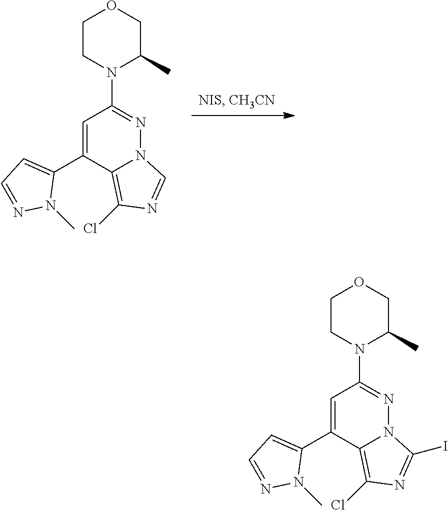

To a solution of (3R)-4-[5-chloro-4-(1-methyl-1H-pyrazol-5-yl)imidazo[1,5-b]pyridazin-2-yl]-3-methylmorpholine (100 mg, 0.30 mmol) in CH₃CN (5 mL) was added NIS (68 mg, 0.30 mmol). The mixture was stirred at room temperature for 2 h. LC-MS showed the reaction was complete. The reaction mixture was diluted with DCM (60 mL), then washed with saturated Na₂S₂O₃ aqueous solution and brine, dried over anhydrous Na₂SO₄, filtered and concentrated. The residue was purified by column chromatography on silica gel (DCM:MeOH=30:1, V/V) to afford the desired product (130 mg, yield: 94%). LC/MS (ESI): m/z 459 [M+H]⁺.

### Step 5. (3R)-4-[5-chloro-4-(1-methyl-1H-pyrazol-5-yl)-7-[1-(oxan-2-yl)-1H-pyrazol-5-yl]imidazo[1,5-b]pyridazin-2-yl]-3-methylmorpholine

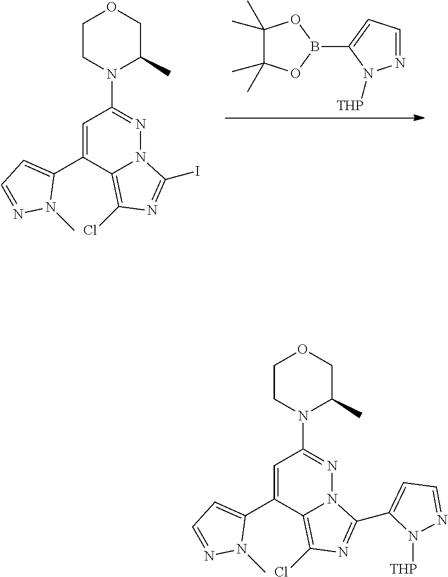

To a solution of (3R)-4-[5-chloro-7-iodo-4-(1-methyl-1H-pyrazol-5-yl)imidazo[1,5-b] pyridazin-2-yl]-3-methylmorpholine (80 mg, 0.17 mmol) in dioxane (5 mL) were added 1-(oxan-2-yl)-5-(tetramethyl-1,3,2-dioxaborolan-2-yl)-1H-pyrazole (121 mg, 0.44 mmol), Pd(PPh₃)₂Cl₂ (25 mg, 0.04 mmol) and K₂CO₃ (2M in H₂O, 0.25 mL, 0.52 mmol). The reaction was stirred at 100° C. overnight under nitrogen atmosphere. LC-MS showed the reaction was complete. The reaction mixture was diluted with DCM (60 mL), then washed with saturated Na₂S₂O₃ aqueous solution and brine, dried over anhydrous Na₂SO₄, filtered and concentrated. The residue was purified by column chromatography on silica gel (DCM:MeOH=20:1, V/V) to afford the desired product (60 mg, yield: 71%). LC/MS (ESI): m/z 483 [M+H]⁺.

### Step 6. (3R)-4-[5-chloro-4-(1-methyl-1H-pyrazol-5-yl)-7-(1H-pyrazol-5-yl)imidazo [1,5-b]pyridazin-2-yl]-3-methylmorpholine

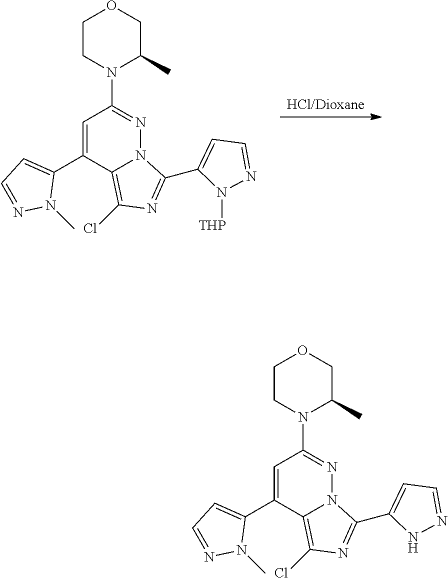

To a solution of (3R)-4-[5-chloro-4-(1-methyl-1H-pyrazol-5-yl)-7-[1-(oxan-2-yl)-1H-pyrazol-5-yl]imidazo[1,5-b]pyridazin-2-yl]-3-methylmorpholine (80 mg, 0.17 mmol) in DCM (2 mL) was added HCl solution (4M in dioxane, 2 mL). The mixture was stirred at room temperature for 1 h. LC-MS showed the reaction was complete. The reaction mixture was concentrated in vacuo. The residue was purified by Pre-HPLC (C₁₈, 10-95%, MeOH in H₂O with 0.1% TFA) to afford the desired product (12 mg, yield: 18%). LC/MS (ESI) m/z: 399 [M+H]⁺. ¹H NMR (400 MHz, DMSO) δ 13.44 (d, J=118.0 Hz, 1H), 7.77 (s, 1H), 7.59 (d, J=1.8 Hz, 1H), 7.14 (d, J=1.9 Hz, 1H), 6.95 (s, 1H), 6.55 (d, J=1.8 Hz, 1H), 4.38 (d, J=5.8 Hz, 1H), 4.06-3.85 (m, 2H), 3.81-3.71 (m, 4H), 3.70 (dd, J=11.5, 2.6 Hz, 1H), 3.63-3.47 (m, 1H), 3.30-3.26 (m, 1H), 1.27 (d, J=6.7 Hz, 3H).

### Example 62

### Synthesis of (3R)-4-[5-chloro-4-(1-methyl-1H-pyrazol-5-yl)-7-(3-methyl-1H-pyrazol-5-yl)imidazo[1,5-b]pyridazin-2-yl]-3-methylmorpholine

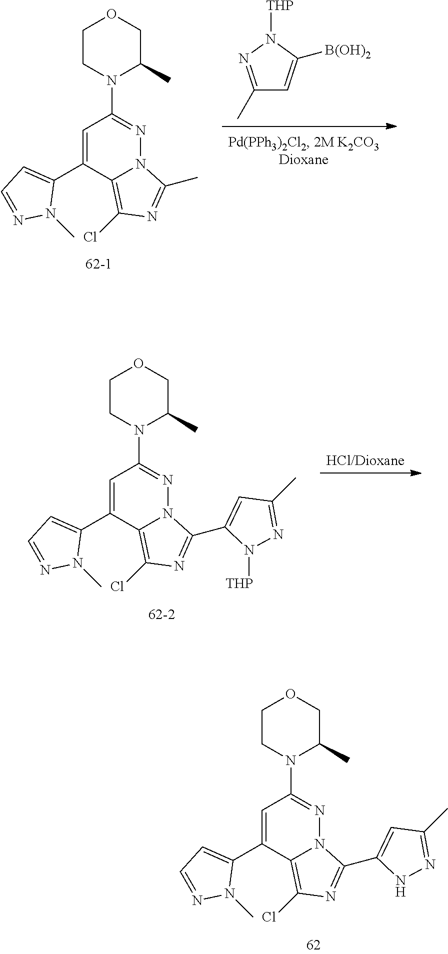

### Step 1. (3R)-4-{5-chloro-7-[3-methyl-1-(oxan-2-yl)-1H-pyrazol-5-yl]-4-(1-methyl-1H-pyrazol-5-yl)imidazo[1,5-b]pyridazin-2-yl}-3-methylmorpholine

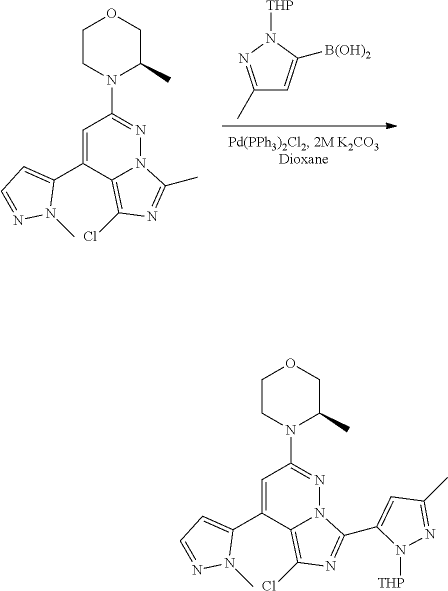

To a solution (3R)-4-[5-chloro-7-iodo-4-(1-methyl-1H-pyrazol-5-yl)imidazo [1,5-b]pyridazin-2-yl]-3-methylmorpholine (60 mg, 0.13 mmol) in dioxane (3 mL) were added [3-methyl-1-(oxan-2-yl)-1H-pyrazol-5-yl]boronic acid (55 mg, 0.26 mmol), Pd(PPh₃)₂Cl₂ (18.4 mg, 0.03 mmol) and K₂CO₃ (54.24 mg, 0.392 mmol). The mixture was stirred at 100° C. overnight under nitrogen atmosphere. LC-MS showed the reaction was complete. The mixture was diluted with EA (40 mL), then washed with water and brine, dried over anhydrous Na₂SO₄, filtered and concentrated. The residue was purified by flash chromatography on silica gel (PE:EA=5:1, V/V) to afford the desired product (40 mg, yield: 61.53%). LC/MS (ESI): m/z 497 [M+H]⁺.

### Step 2. (3R)-4-[5-chloro-4-(1-methyl-1H-pyrazol-5-yl)-7-(3-methyl-1H-pyrazol-5-yl) imidazo[1,5-b]pyridazin-2-yl]-3-methylmorpholine

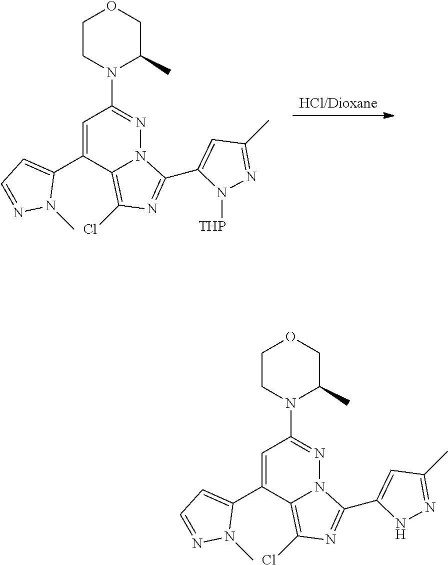

To a solution of (3R)-4-{5-chloro-7-[3-methyl-1-(oxan-2-yl)-1H-pyrazol-5-yl]-4-(1-methyl-1H-pyrazol-5-yl)imidazo[1,5-b]pyridazin-2-yl}-3-methylmorpholine (140 mg, 0.28 mmol) in DCM (5 mL) was added HCl solution (4M in dioxane, 5 mL). The mixture was stirred at room temperature for 1 h. LC-MS showed the reaction was complete. The reaction mixture was concentrated under vacuo. The residue was purified by Prep-HPLC (C₁₈, 10-95%, MeOH in H₂O with 0.1% HCOOH) to give the desired product (33 mg, yield: 28.37%). LC/MS (ESI): m/z 413 [M+H]⁺. ¹H NMR (400 MHz, DMSO) δ 13.03 (d, J=106.2 Hz, 1H), 7.59 (d, J=1.9 Hz, 1H), 6.95 (s, 1H), 6.88 (s, 1H), 6.54 (d, J=1.8 Hz, 1H), 4.36 (s, 1H), 4.07-3.85 (m, 2H), 3.86-3.73 (m, 4H), 3.70 (dd, J=11.5, 2.7 Hz, 1H), 3.64-3.45 (m, 1H), 3.29 (s, 1H), 2.32 (d, J=15.9 Hz, 3H), 1.26 (t, J=6.3 Hz, 3H).

### Example 63

### Synthesis of (R)-1-(7-(3-methyl-1H-pyrazol-5-yl)-2-(3-methylmorpholino)imidazo[1,5-b]pyridazin-4-yl)cyclopropane-1-carbonitrile

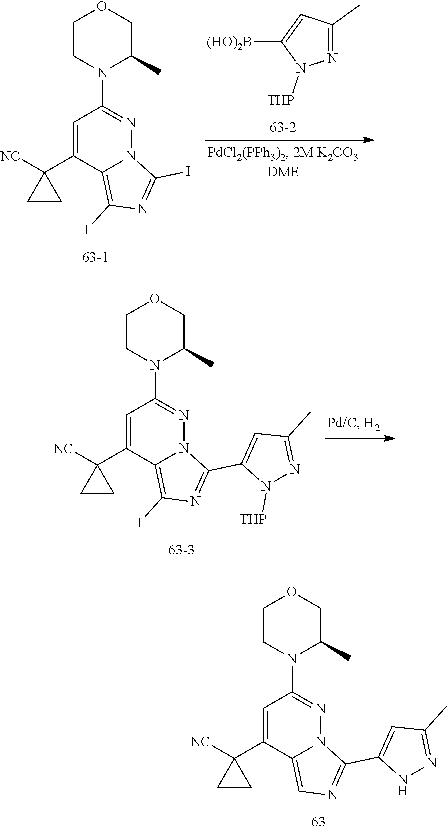

### Step 1. 1-(5-iodo-7-(3-methyl-1-(tetrahydro-2H-pyran-2-yl)-1H-pyrazol-5-yl)-2-((R)-3-methylmorpholino)imidazo[1,5-b]pyridazin-4-yl)cyclopropane-1-carbonitrile

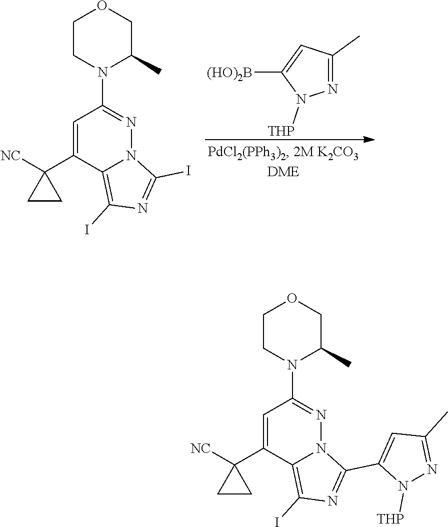

A mixture of 1-{5,7-diiodo-2-[(3R)-3-methylmorpholin-4-yl]imidazo[1,5-b]pyridazin-4-yl}cyclopropane-1-carbonitrile (200 mg, 0.37 mmol), [3-methyl-1-(oxan-2-yl)-1H-pyrazol-5-yl]boronic acid (157 mg, 0.74 mmol), Pd(dppf)Cl₂ (50 mg, 0.07 mmol) and K₂CO₃ (2.0 M in H₂O, 0.5 mL, 1.0 mmol) in DME (5 mL) was stirred at 100° C. for 16 h under N₂ atmosphere. LC-MS showed the reaction was complete. The reaction mixture was diluted with H₂O (20 mL), then extracted with EA (20 mL×3). The combined organic layer was washed with brine, dried over anhydrous Na₂SO₄, filtered and concentrated. The residue was purified by column chromatography on silica gel (DCM:MeOH=20:1, V/V) to give the desired product (60 mg, yield: 28%). LC/MS (ESI): m/z 574 [M+H]⁺.

### Step 2. (R)-1-(7-(3-methyl-1H-pyrazol-5-yl)-2-(3-methylmorpholino)imidazo[1,5-b]pyridazin-4-yl)cyclopropane-1-carbonitrile

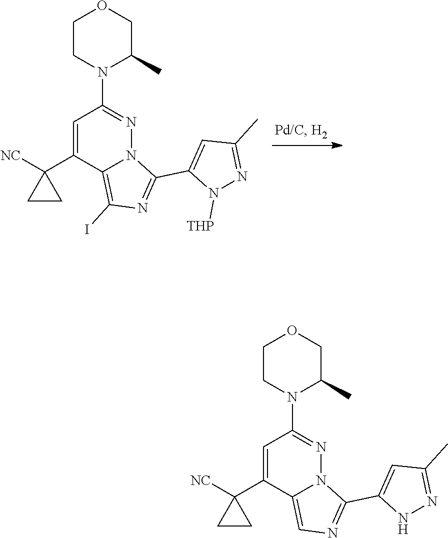

A mixture of 1-{5-iodo-7-[3-methyl-1-(oxan-2-yl)-1H-pyrazol-5-yl]-2-[(3S)-3-methyl morpholin-4-yl]imidazo[1,5-b]pyridazin-4-yl}cyclopropane-1-carbonitrile (92 mg, 0.16 mmol) and Pd/C (10%, 40 mg) in MeOH (5 mL) was stirred at 30° C. for 16 h under H₂ atmosphere. LC-MS showed the reaction was complete. The reaction mixture was filtered, then the filtrate was concentrated under reduced pressure to dryness. The residue was purified by Prep-HPLC (C18, 10-95%, MeOH in H₂O with 0.1% HCOOH) to give the desired product (9 mg, yield: 15%). LC/MS (ESI): m/z 448 [M+H]⁺. ¹H NMR (400 MHz, DMSO) δ 12.97 (s, 1H), 7.68 (s, 1H), 6.84 (s, 1H), 6.78 (s, 1H), 4.36 (d, J=6.0 Hz, 1H), 4.00 (dd, J=11.4, 3.2 Hz, 1H), 3.88 (d, J=12.6 Hz, 1H), 3.77 (d, J=11.3 Hz, 1H), 3.69 (dd, J=11.4, 2.8 Hz, 1H), 3.54 (td, J=11.7, 2.8 Hz, 1H), 3.25 (td, J=12.9, 3.7 Hz, 1H), 2.28 (s, 3H), 1.84-1.71 (m, 4H), 1.23 (d, J=6.7 Hz, 3H).

### Example 64

### Synthesis of (R)-2-methyl-2-(7-(3-methyl-1H-pyrazol-5-yl)-2-(3-methylmorpholino) imidazo[1,5-b]pyridazin-4-yl)propanenitrile

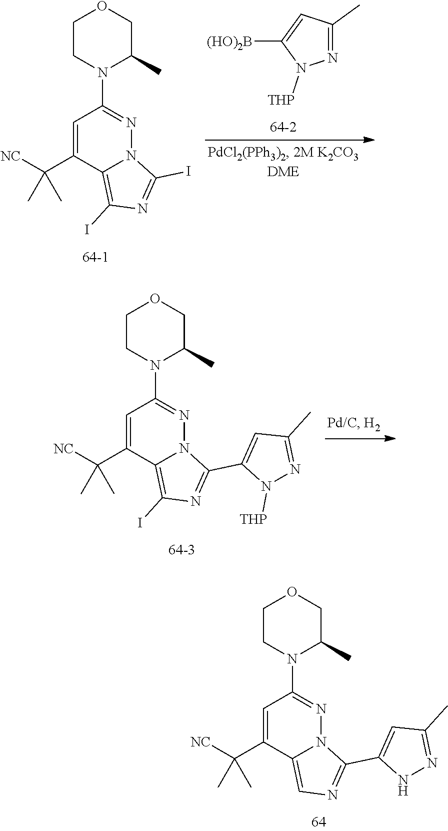

### Step 1. 2-(5-iodo-7-(3-methyl-1-(tetrahydro-2H-pyran-2-yl)-1H-pyrazol-5-yl)-2-((R)-3-methylmorpholino)imidazo[1,5-b]pyridazin-4-yl)-2-methylpropanenitrile

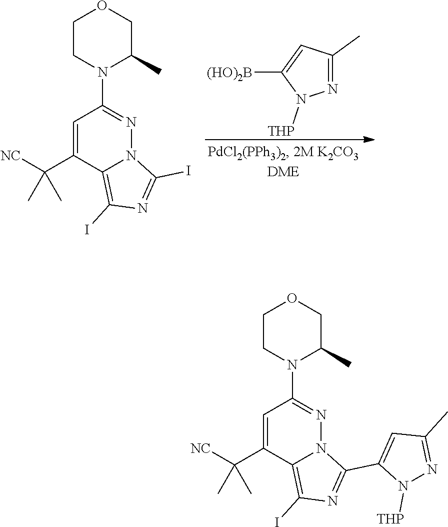

A mixture of 2-{5,7-diiodo-2-[(3R)-3-methylmorpholin-4-yl]imidazo[1,5-b]pyridazin-4-yl}-2-methylpropanenitrile (200 mg, 0.37 mmol), [3-methyl-1-(oxan-2-yl)-1H-pyrazol-5-yl]boronic acid (156 mg, 0.74 mmol), Pd(dppf)Cl₂ (50 mg, 0.07 mmol) and K₂CO₃ (2.0 M in H₂O, 0.5 mL, 1.0 mmol) in DME (5 mL) was stirred at 100° C. for 16 h under N₂ atmosphere. LC-MS showed the reaction was complete. The reaction mixture was diluted with H₂O (20 mL), then extracted with EA (20 mL×3). The combined organic layer was washed with brine, dried over anhydrous Na₂SO₄, filtered and concentrated. The residue was purified by column chromatography on silica gel (DCM:MeOH=20:1, V/V) to give the desired product (60 mg, yield: 28%). LC/MS (ESI): m/z 576 [M+H]⁺.

### Step 2. (R)-2-methyl-2-(7-(3-methyl-1H-pyrazol-5-yl)-2-(3-methylmorpholino) imidazo[1,5-b]pyridazin-4-yl)propanenitrile

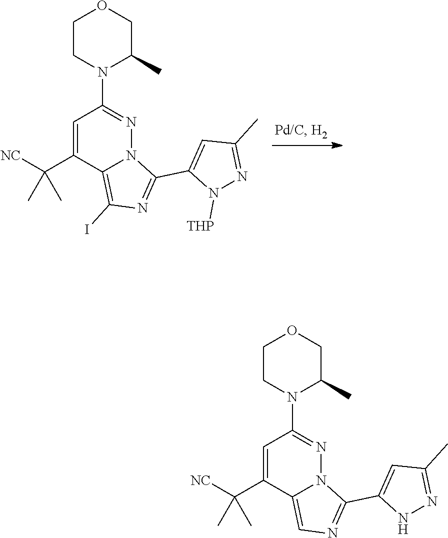

A mixture of 2-{5-iodo-7-[3-methyl-1-(oxan-2-yl)-1H-pyrazol-5-yl]-2-[(3R)-3-methyl morpholin-4-yl]imidazo[1,5-b]pyridazin-4-yl}-2-methylpropanenitrile (120 mg, 0.21 mmol) and Pd/C (10%, 60 mg) in MeOH (6 mL) was stirred at 30° C. for 16 h under H₂ atmosphere. LC-MS showed the reaction was complete. The reaction mixture was filtered, then the filtrate was concentrated under reduced pressure to dryness. The residue was purified by Prep-HPLC (C18, 10-95%, MeOH in H₂O with 0.1% HCOOH) to give the desired product (10 mg, yield: 13%). LC/MS (ESI): m/z 366 [M+H]⁺. ¹H NMR (400 MHz, DMSO) δ 12.93 (d, J=104.0 Hz, 1H), 7.75 (s, 1H), 6.85 (s, 1H), 6.72 (s, 1H), 4.34 (d, J=6.9 Hz, 1H), 4.01 (dd, J=11.2, 2.4 Hz, 1H), 3.91-3.67 (m, 3H), 3.56 (td, J=11.7, 2.7 Hz, 1H), 3.29-3.21 (m, 1H), 2.29 (s, 3H), 1.87 (s, 6H), 1.24 (t, J=8.0 Hz, 3H).

### Example 65

### Synthesis of 3-[5-methyl-4-(1-methyl-1H-pyrazol-5-yl)-7-(1H-pyrazol-5-yl)imidazo [1,5-b]pyridazin-2-yl]-8-oxa-3-azabicyclo[3.2.1]octane

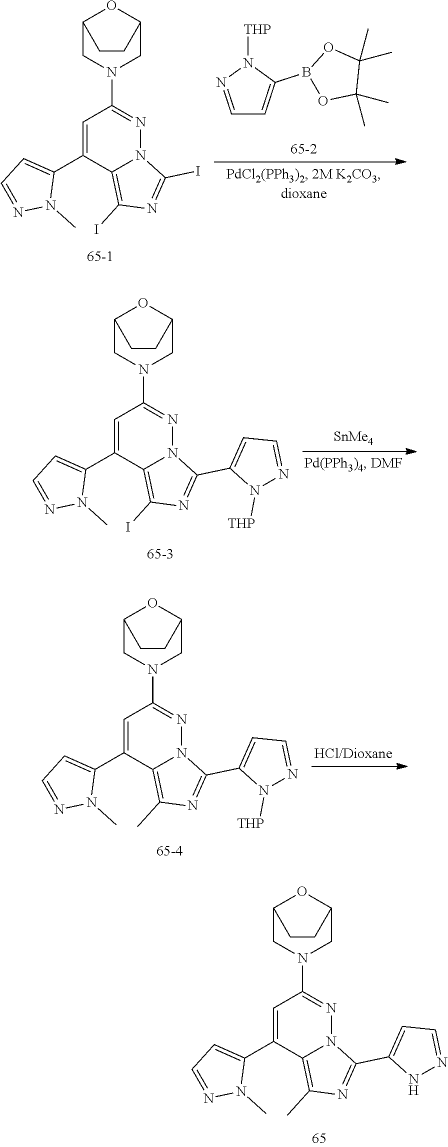

### Step 1.3-[5-iodo-4-(1-methyl-1H-pyrazol-5-yl)-7-[1-(oxan-2-yl)-1H-pyrazol-5-yl]imidazo[1,5-b]pyridazin-2-yl]-8-oxa-3-azabicyclo[3.2.1]octane

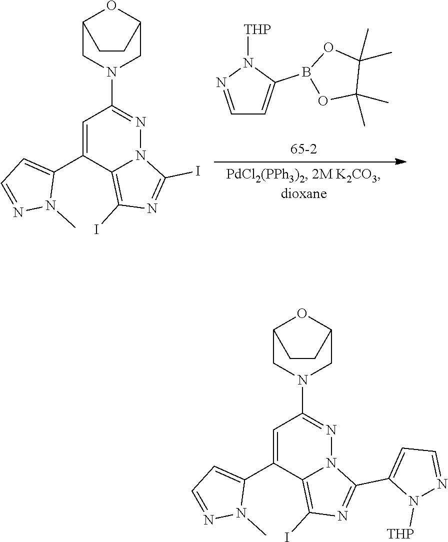

To a solution of 3-[5,7-diiodo-4-(1-methyl-1H-pyrazol-5-yl)imidazo[1,5-b]pyridazin-2-yl]-8-oxa-3-azabicyclo[3.2.1]octane (400 mg, 0.71 mmol) in dioxane (10 mL) was added 1-(oxan-2-yl)-5-(tetramethyl-1,3,2-dioxaborolan-2-yl)-1H-pyrazole (594 mg, 2.14 mmol), Pd(PPh₃)₂Cl₂ (100 mg, 0.14 mmol) and K₂CO₃ (295 mg, 2.14 mmol). The mixture was stirred at 100° C. overnight under nitrogen atmosphere. LC-MS showed the reaction was complete. The reaction mixture was diluted with EA (50 mL), then washed with water and brine, dried over anhydrous Na₂SO₄, filtered and concentrated. The residue was purified by column chromatography on silica gel (DCM:MOH=40:1, V/V) to afford the desired product (340 mg, yield: 81.48%). LC/MS (ESI): m/z 587 [M+H]⁺.

### Step 2. 3-[5-methyl-4-(1-methyl-1H-pyrazol-5-yl)-7-[1-(oxan-2-yl)-1H-pyrazol-5-yl] imidazo[1,5-b]pyridazin-2-yl]-8-oxa-3-azabicyclo[3.2.1]octane

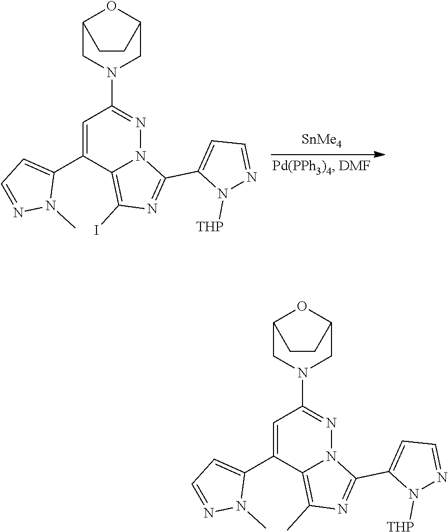

To a solution of 3-[5-iodo-4-(1-methyl-1H-pyrazol-5-yl)-7-[1-(oxan-2-yl)-1H-pyrazol-5-yl]imidazo[1,5-b]pyridazin-2-yl]-8-oxa-3-azabicyclo[3.2.1]octane (200 mg, 0.34 mmol) in DMF (10 ml) were added Sn(CH₃)₄ (0.31 mL, 1.71 mmol) and Pd(PPh₃)₄ (78.8 mg, 0.07 mmol). The mixture was stirred at 100° C. overnight under N₂ atmosphere. LC-MS showed the reaction was complete. The reaction mixture was diluted with EA (50 mL), then washed with water and brine, dried over anhydrous Na₂SO₄, filtered and concentrated. The residue was purified by column chromatography on silica gel (DCM:MOH=40:1, V/V) to afford the desired product (105 mg, yield: 64.88%). LC/MS (ESI): m/z 475 [M+H]⁺.

### Step 3. 3-[5-methyl-4-(1-methyl-1H-pyrazol-5-yl)-7-(1H-pyrazol-5-yl)imidazo[1,5-b] pyridazin-2-yl]-8-oxa-3-azabicyclo[3.2.1]octane

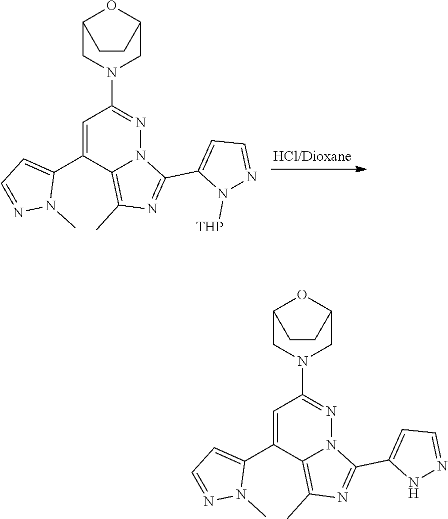

To a solution of 3-[5-methyl-4-(1-methyl-1H-pyrazol-5-yl)-7-[1-(oxan-2-yl)-1H-pyrazol-5-yl]imidazo[1,5-b]pyridazin-2-yl]-8-oxa-3-azabicyclo[3.2.1]octane (100 mg, 0.21 mmol) in DCM (5 mL) was added HCl solution (4M in Dioxane, 5 mL). The mixture was stirred at room temperature for 1 h. LC-MS showed the reaction was complete. The reaction mixture was concentrated under reduced pressure. The residue was purified by Prep-HPLC (C18, 10-95%, MeOH in H₂O with 0.1% HCOOH) to give the desired product (14 mg, yield: 17.02%). LC/MS (ESI): m/z 391 [M+H]⁺. ¹H NMR (400 MHz, DMSO) δ 7.70 (t, J=46.0 Hz, 2H), 7.11 (s, 1H), 6.74 (s, 1H), 6.55 (d, J=1.7 Hz, 1H), 4.48 (s, 2H), 3.88 (d, J=12.1 Hz, 2H), 3.74 (s, 3H), 3.15 (d, J=11.7 Hz, 2H), 1.91 (d, J=15.0 Hz, 3H), 1.84 (d, J=8.1 Hz, 4H).

### Example 66

### Synthesis of 3-[5-methyl-4-(1-methyl-1H-pyrazol-5-yl)-7-(3-methyl-1H-pyrazol-5-yl) imidazo[1,5-b]pyridazin-2-yl]-8-oxa-3-azabicyclo[3.2.1]octane

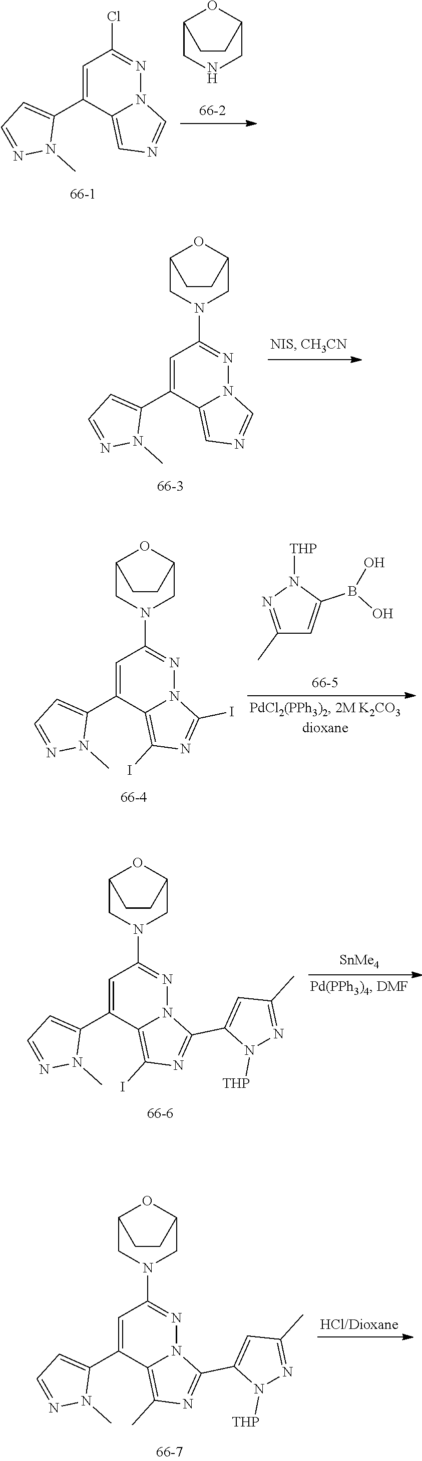

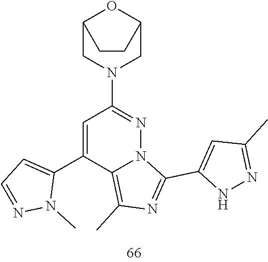 [SMILES: Cc1cc(-c2nc(C)c3c(-c4ccnn4C)cc(N4CC5CCC(C4)O5)nn23)[nH]n1]

### Step 1.3-[4-(1-methyl-1H-pyrazol-5-yl)imidazo[1,5-b]pyridazin-2-yl]-8-oxa-3-azabicyclo[3.2.1]octane

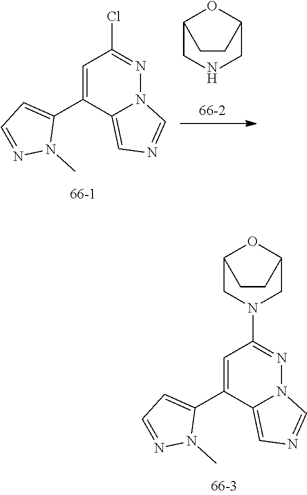

To a solution of 5-{2-chloroimidazo[1,5-b]pyridazin-4-yl}-1-methyl-1H-pyrazole (1 g, 4.28 mmol) in NMP (10 mL) were added 8-oxa-3-azabicyclo[3.2.1]octane (1.45 g, 12.84 mmol) and DIPEA (1.66 g, 12.84 mmol). The mixture was stirred at 180° C. for 8 h. LC-MS showed the reaction was complete. The reaction mixture was diluted with EA (60 mL), then washed with water and brine, dried over anhydrous Na₂SO₄, filtered and concentrated. The residue was purified by column chromatography on silica gel (DCM:MOH=40:1, V/V) to afford the desired product (1.14 g, yield: 85.83%). LC/MS (ESI): m/z 311 [M+H]⁺.

### Step 2.3-[5,7-diiodo-4-(1-methyl-1H-pyrazol-5-yl)imidazo[1,5-b]pyridazin-2-yl]-8-oxa-3-azabicyclo[3.2.1]octane

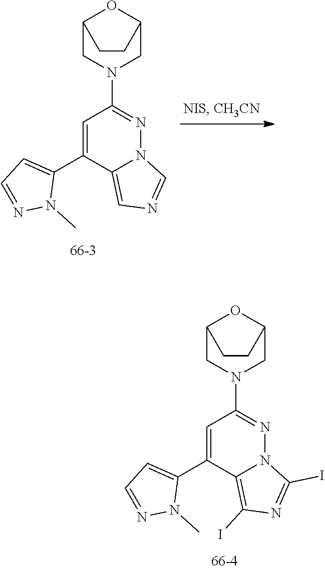 [SMILES: Cn1nccc1-c1cc(N2CC3CCC(C2)O3)nn2cncc12 ; Cn1nccc1-c1cc(N2CC3CCC(C2)O3)nn2c(I)nc(I)c12]

To a solution of 3-[4-(1-methyl-1H-pyrazol-5-yl)imidazo[1,5-b]pyridazin-2-yl]-8-oxa-3-azabicyclo[3.2.1]octane (1.13 g, 3.64 mmol) in CH₃CN (30 ml) was added NIS (1.89 g, 10.923 mmol) portion wise. The mixture was stirred at room temperature for 2 h. LC-MS showed the reaction was complete. The reaction mixture was diluted with EA (60 mL), then washed with saturated Na₂S₂O₃ aqueous solution and brine, dried over anhydrous Na₂SO₄, filtered and concentrated. The residue was purified by column chromatography on silica gel (DCM:MOH=40:1, V/V) to afford the desired product (1.7 g, yield: 83.06%). LC/MS (ESI): m/z 563 [M+H]⁺.

### Step 3. 3-{5-iodo-7-[3-methyl-1-(oxan-2-yl)-1H-pyrazol-5-yl]-4-(1-methyl-1H-pyrazol-5-yl)imidazo[1,5-b]pyridazin-2-yl}-8-oxa-3-azabicyclo[3.2.1]octane

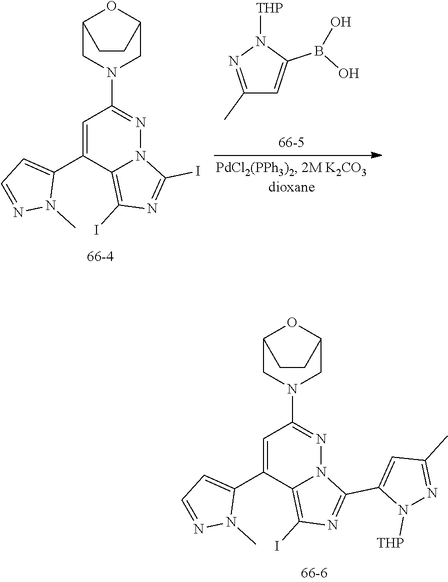

To a solution of 3-[5,7-diiodo-4-(1-methyl-1H-pyrazol-5-yl)imidazo[1,5-b]pyridazin-2-yl]-8-oxa-3-azabicyclo[3.2.1]octane (40 mg, 0.71 mmol) in dioxane (10 mL) were added [3-methyl-1-(oxan-2-yl)-1H-pyrazol-5-yl]boronic acid (449 mg, 2.14 mmol), Pd(PPh₃)₂Cl₂ (100 mg, 0.14 mmol) and K₂CO₃ (295 mg, 2.14 mmol). The mixture was stirred at 100° C. overnight under nitrogen atmosphere. LC-MS showed the reaction was complete. The reaction mixture was diluted with EA (60 mL), then washed with saturated Na₂S₂O₃ aqueous solution and brine, dried over anhydrous Na₂SO₄, filtered and concentrated. The residue was purified by column chromatography on silica gel (DCM:MOH=30:1, V/V) to afford the desired product (315 mg, yield: 73.72%). LC/MS (ESI): m/z 601 [M+H]⁺.

### Step 4. 3-{5-methyl-7-[3-methyl-1-(oxan-2-yl)-1H-pyrazol-5-yl]-4-(1-methyl-1H-pyrazol-5-yl)imidazo[1,5-b]pyridazin-2-yl}-8-oxa-3-azabicyclo[3.2.1]octane

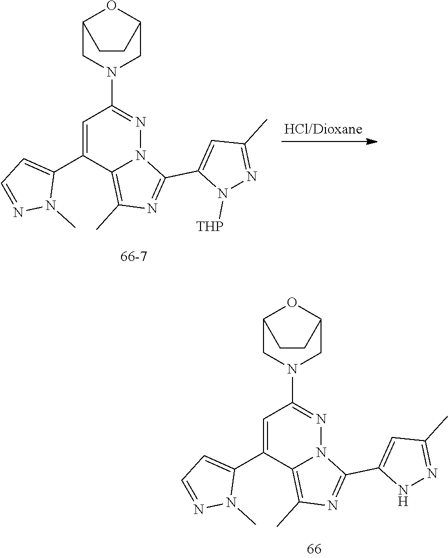

To a solution of 3-{5-iodo-7-[3-methyl-1-(oxan-2-yl)-1H-pyrazol-5-yl]-4-(1-methyl-1H-pyrazol-5-yl)imidazo[1,5-b]pyridazin-2-yl}-8-oxa-3-azabicyclo[3.2.1]octane (200 mg, 0.33 mmol) in DMF (10 mL) were added Sn(CH₃)₄ (0.31 mL, 1.67 mmol) and Pd(PPh₃)₄ (77 mg, 0.07 mmol). The mixture was stirred at 100° C. overnight under N₂ atmosphere. LC-MS showed the reaction was complete. The reaction mixture was diluted with EA (60 mL), then washed with water and brine, dried over anhydrous Na₂SO₄, filtered and concentrated. The residue was purified by column chromatography on silica gel (DCM:MOH=20:1, V/V) to afford the desired product (140 mg, yield: 86.03%). LC/MS (ESI): m/z 489 [M+H]⁺.

### Step 5. 3-[5-methyl-4-(1-methyl-1H-pyrazol-5-yl)-7-(3-methyl-1H-pyrazol-5-yl)imidazo[1,5-b]pyridazin-2-yl]-8-oxa-3-azabicyclo[3.2.1]octane

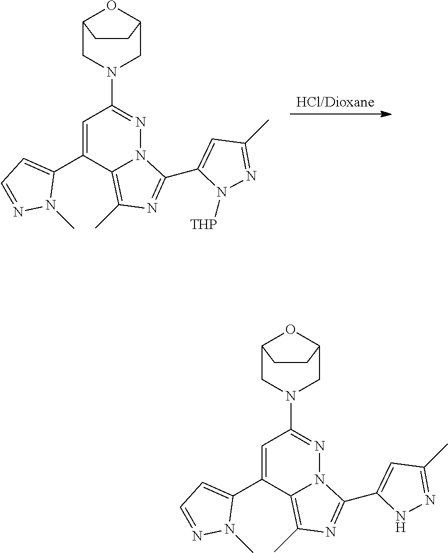

To a solution of 3-{5-methyl-7-[3-methyl-1-(oxan-2-yl)-1H-pyrazol-5-yl]-4-(1-methyl-1H-pyrazol-5-yl)imidazo[1,5-b]pyridazin-2-yl}-8-oxa-3-azabicyclo[3.2.1]octane (140 mg, 0.29 mmol) in DCM (7 mL) was added HCl solution (4M in Dioxane, 7 mL). The mixture was stirred at room temperature for 1 h. LC-MS showed the reaction was complete. The reaction mixture was concentrated under reduced pressure. The residue was purified by Prep-HPLC (C18, 10-95%, MeOH in H₂O with 0.1% HCOOH) to give the desired product (15 mg, yield: 12.94%). LC/MS (ESI): m/z 405 [M+H]⁺. ¹H NMR (400 MHz, DMSO) δ 12.89 (s, 1H), 7.62 (d, J=1.8 Hz, 1H), 6.85 (s, 1H), 6.71 (s, 1H), 6.54 (d, J=1.8 Hz, 1H), 4.48 (s, 2H), 3.87 (d, J=12.4 Hz, 2H), 3.74 (s, 3H), 3.14 (d, J=10.7 Hz, 2H), 2.29 (s, 3H), 1.91 (s, 3H), 1.86 (s, 4H).

### Example 67

### Synthesis of (R)-2-(3-(3-methyl-1H-pyrazol-5-yl)-5-(3-methylmorpholino)isothiazolo[4,5-b]pyridin-7-yl)propan-2-ol

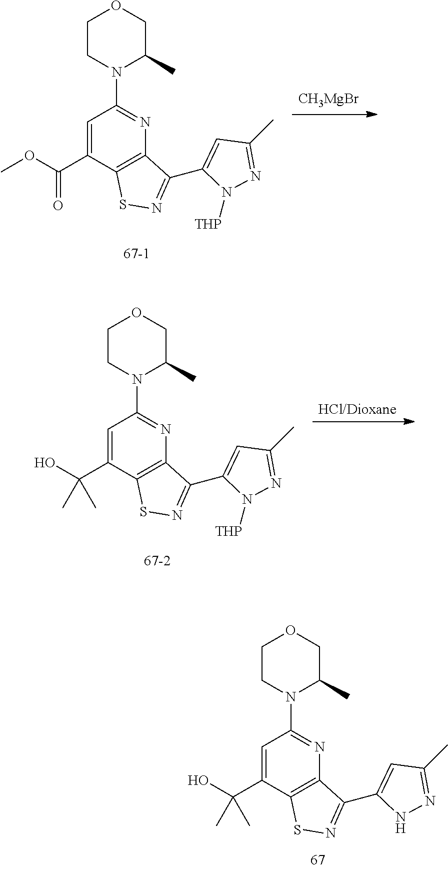

### Step 1. 2-(3-(3-methyl-1-(tetrahydro-2H-pyran-2-yl)-1H-pyrazol-5-yl)-5-((R)-3-methylmorpholino)isothiazolo[4,5-b]pyridin-7-yl)propan-2-ol

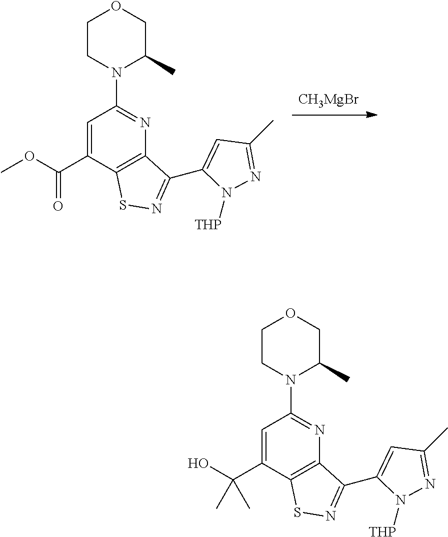

To solution of methyl 3-(3-methyl-1-(tetrahydro-2H-pyran-2-yl)-1H-pyrazol-5-yl)-5-((R)-3-methylmorpholino)isothiazolo[4,5-b]pyridine-7-carboxylate (70 mg, 0.15 mmol) in THF (5 mL) at 0° C. was added Methyl magnesium bromide (3M in ethyl ether, 0.15 mL, 0.46 mmol) drop wise. After stirring at 0° C. for 30 min, the mixture was warmed to room temperature and stirred for an additional 1 h. LC-MS showed the reaction was complete. The reaction mixture was quenched with saturated NH₄Cl aqueous solution and diluted with EA (30 mL). The organic layer was separated, then washed with brine, dried over anhydrous Na₂SO₄, filtered and concentrated. The residue was purified by column chromatography on silica gel (PE:EA=1:1, V/V) to afford the desired product (35 mg, yield: 50%). LC/MS (ESI): m/z 458 [M+H]⁺.

### Step 2. (R)-2-(3-(3-methyl-1H-pyrazol-5-yl)-5-(3-methylmorpholino)isothiazolo[4,5-b]pyridin-7-yl)propan-2-ol

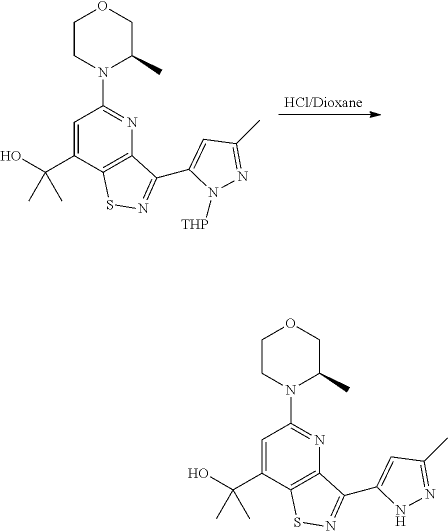

A mixture of 2-(3-(3-methyl-1-(tetrahydro-2H-pyran-2-yl)-1H-pyrazol-5-yl)-5-((R)-3-methylmorpholino)isothiazolo[4,5-b]pyridin-7-yl)propan-2-ol (30 mg, 0.07 mmol) in HCl solution (4M in Dioxane, 2 mL) was stirred at room temperature for 1 h. LC-MS showed the reaction was complete. The reaction mixture was concentrated in vacuo. The residue was purified by Pre-HPLC (C₁₈, 10-95%, MeOH in H₂O with 0.1% HCOOH) to afford the desired product (17 mg, yield: 69.42%). LC/MS (ESI) m/z: 374 [M+H]⁺. ¹H NMR (400 MHz, DMSO) δ 12.95 (d, J=103.8 Hz, 1H), 7.09 (s, 1H), 7.02 (s, 1H), 6.06 (s, 1H), 4.53 (s, 1H), 4.09 (d, J=12.9 Hz, 1H), 4.02 (d, J=9.1 Hz, 1H), 3.81 (d, J=11.3 Hz, 1H), 3.72 (d, J=11.1 Hz, 1H), 3.57 (t, J=10.8 Hz, 1H), 3.22 (t, J=11.1 Hz, 1H), 2.29 (s, 3H), 1.56 (s, 6H), 1.21 (d, J=6.6 Hz, 3H).

### Example 68

### Synthesis of (R)-3-methyl-4-(7-(1-methyl-1H-1,2,3-triazol-5-yl)-3-(3-methyl-1H-pyrazol-5-yl)isothiazolo[4,5-b]pyridin-5-yl)morpholine

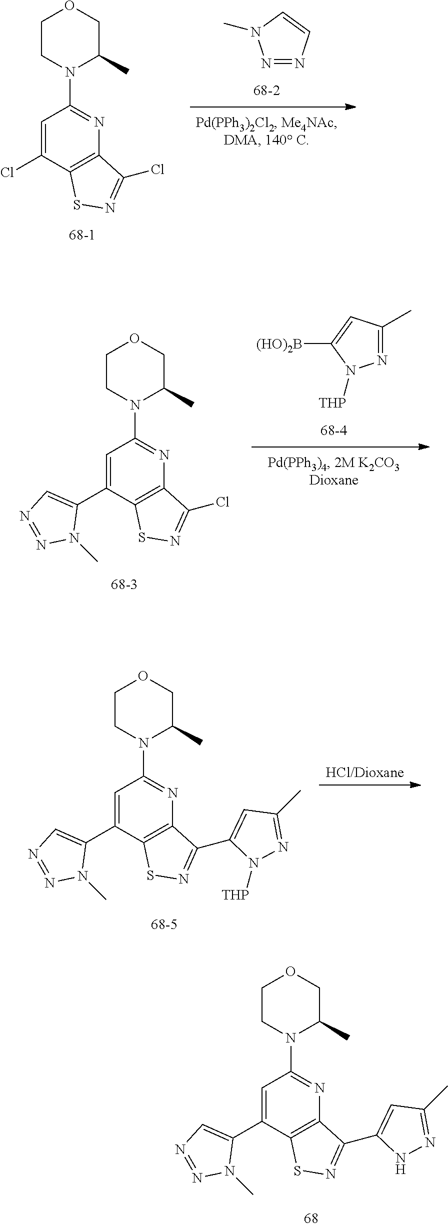

### Step 1. (R)-4-(3-chloro-7-(1-methyl-1H-1,2,3-triazol-5-yl)isothiazolo[4,5-b]pyridin-5-yl)-3-methylmorpholine

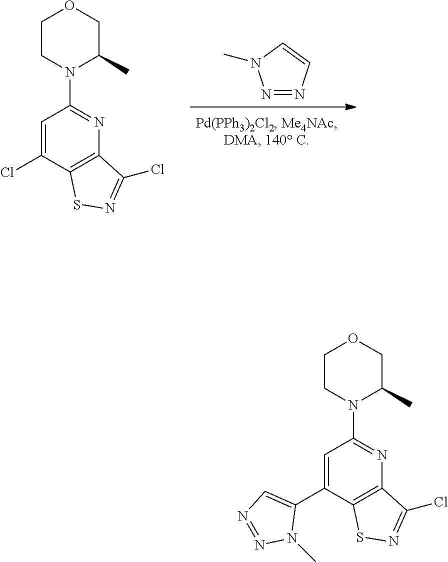

To a mixture of (3R)-4-{3,7-dichloro-[1,2]thiazolo[4,5-b]pyridin-5-yl}-3-methyl morpholine (250 mg, 0.82 mmol), 1-methyl-1H-1,2,3-triazole (410 mg, 4.93 mmol) and Me₄NAc (289 mg, 2.46 mmol) in DMA (10 mL) was added Pd(PPh₃)₂Cl₂ (115 mg, 0.164 mmol). The mixture was stirred at 140° C. for 12 h under N₂ atmosphere. LC-MS showed the reaction was complete. The mixture was poured into H₂O and extracted with EA (30 mL×3). The combined organic phase was washed with brine, dried over anhydrous Na₂SO₄, filtered and concentrated to dryness. The residue was purified by column chromatography on silica gel (PE:EA=1:1, V/V) to give the desired product (200 mg, yield: 69%). LC/MS (ESI): m/z 351 [M+H]⁺.

### Step 2. (3R)-3-methyl-4-(3-(3-methyl-1-(tetrahydro-2H-pyran-2-yl)-1H-pyrazol-5-yl)-7-(1-methyl-1H-1,2,3-triazol-5-yl)isothiazolo[4,5-b]pyridin-5-yl)morpholine

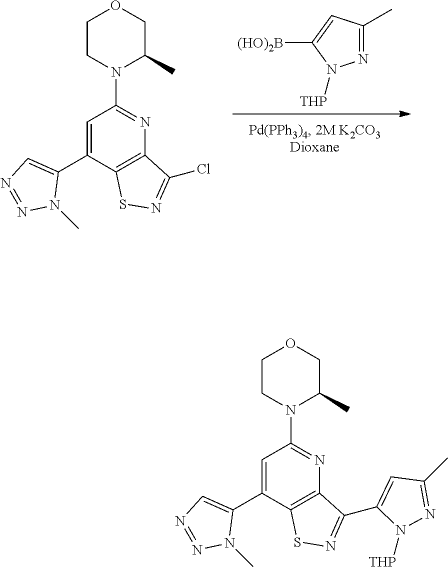

To a mixture of (R)-4-(3-chloro-7-(1-methyl-1H-1,2,3-triazol-5-yl)isothiazolo[4,5-b]pyridin-5-yl)-3-methylmorpholine (100 mg, 0.29 mmol), [3-methyl-1-(oxan-2-yl)-1H-pyrazol-5-yl]boronic acid (180 mg, 0.86 mmol) and K₂CO₃ (2M in H₂O, 0.7 mL, 1.42 mmol) in dioxane (8 mL) was added tetrakis (triphenylphosphane) palladium (66 mg, 0.06 mmol). The mixture was stirred at 100° C. for 16 h under N₂ atmosphere. LC-MS showed the reaction was complete. The reaction mixture was diluted with EA (50 mL), then washed with water and brine, dried over anhydrous Na₂SO₄, filtered and concentrated. The residue was purified by column chromatography on silica gel (PE:EA=1:1, V/V) to give the desired product (60 mg, yield: 44%). LC/MS ESI (m/z): 481 [M+H]⁺.

### Step 3. (R)-3-methyl-4-(7-(1-methyl-1H-1,2,3-triazol-5-yl)-3-(3-methyl-1H-pyrazol-5-yl)isothiazolo[4,5-b]pyridin-5-yl)morpholine

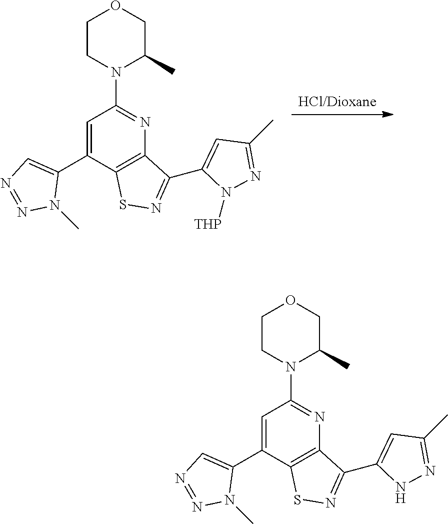

To a mixture of (3R)-3-methyl-4-(3-(3-methyl-1-(tetrahydro-2H-pyran-2-yl)-1H-pyrazol-5-yl)-7-(1-methyl-1H-1,2,3-triazol-5-yl)isothiazolo[4,5-b]pyridin-5-yl)morpholine (60 mg, 0.13 mmol) in DCM (0.5 mL) was added HCl solution (4 M in dioxane, 1.5 mL). The mixture was stirred at room temperature for 1 h. LC-MS showed the reaction was complete. The mixture was concentrated to dryness. The residue was purified by Prep-HPLC (C₁₈, 10-95%, MeCN in H₂O with 0.1% HCOOH) to give the desired product (18 mg, yield: 36%). LC/MS (ESI): m/z 397 [M+H]⁺. ¹H NMR (400 MHz, DMSO) δ 13.12 (d, J=127.1 Hz, 1H), 8.25 (s, 1H), 7.50 (s, 1H), 7.16 (s, 1H), 4.61-4.53 (m, 1H), 4.21 (s, 3H), 4.20-4.14 (m, 1H), 4.06 (d, J=10.3 Hz, 1H), 3.83 (d, J=11.3 Hz, 1H), 3.77-3.71 (m, 1H), 3.63-3.54 (m, 1H), 3.31-3.23 (m, 1H), 2.32 (s, 3H), 1.27 (d, J=6.6 Hz, 3H).

### Example 69

### Synthesis of (R)-3-methyl-4-(7-(1-methyl-1H-1,2,3-triazol-5-yl)-3-(1H-pyrazol-5-yl)isothiazolo[4,5-b]pyridin-5-yl)morpholine

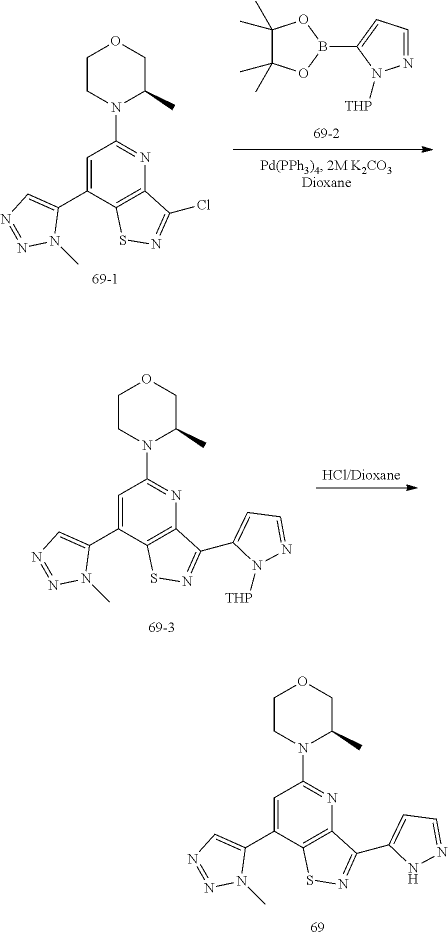

### Step 1. (3R)-3-methyl-4-(7-(1-methyl-1H-1,2,3-triazol-5-yl)-3-(1-(tetrahydro-2H-pyran-2-yl)-1H-pyrazol-5-yl)isothiazolo[4,5-b]pyridin-5-yl)morpholine

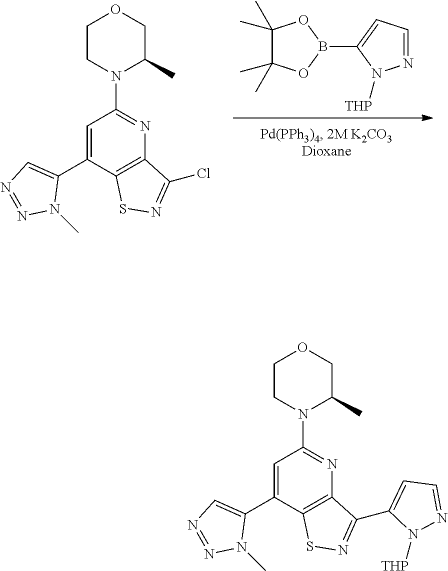

To a mixture of (R)-4-(3-chloro-7-(1-methyl-1H-1,2,3-triazol-5-yl)isothiazolo[4,5-b]pyridin-5-yl)-3-methylmorpholine (95 mg, 0.27 mmol), 1-(oxan-2-yl)-5-(tetra methyl-1,3,2-dioxaborolan-2-yl)-1H-pyrazole (226 mg, 0.81 mmol) and K₂CO₃ (2M in H₂O, 0.68 mL, 1.36 mmol) in dioxane (8 mL) was added Pd(PPh₃)₄ (63 mg, 0.05 mmol). The mixture was stirred at 100° C. for 16 h under N₂ atmosphere. LC-MS showed the reaction was complete. The reaction mixture was diluted with EA (50 mL), then washed with water and brine, dried over anhydrous Na₂SO₄, filtered and concentrated. The residue was purified by column chromatography on silica gel (PE:EA=1:1, V/V) to give the desired product (52 mg, yield: 41%). LC/MS ESI (m/z): 467 [M+H]⁺.

### Step 2. (R)-3-methyl-4-(7-(1-methyl-1H-1,2,3-triazol-5-yl)-3-(1H-pyrazol-5-yl)isothiazolo[4,5-b]pyridin-5-yl)morpholine

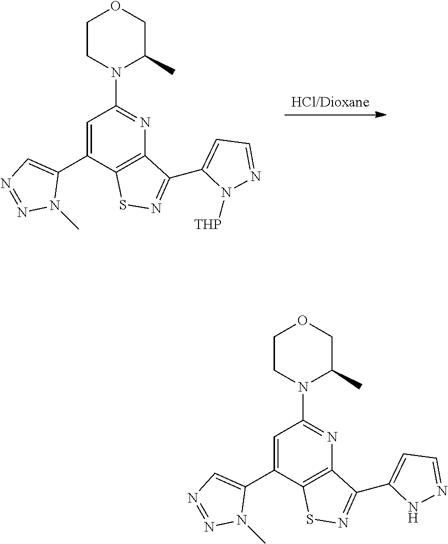

To a mixture of (3R)-3-methyl-4-(7-(1-methyl-1H-1,2,3-triazol-5-yl)-3-(1-(tetrahydro-2H-pyran-2-yl)-1H-pyrazol-5-yl)isothiazolo[4,5-b]pyridin-5-yl)morpholine (52 mg, 0.11 mmol) in DCM (0.5 mL) was added HCl solution (4M in dioxane, 1.5 mL). The mixture was stirred at room temperature for 1 h. LC-MS showed the reaction was complete. The mixture was concentrated to dryness. The residue was purified by Prep-HPLC (C₁₈, 10-95%, MeCN in H₂O with 0.1% HCOOH) to give the desired product (8 mg, yield: 19%). LC/MS (ESI): m/z 383 [M+H]⁺. ¹H NMR (400 MHz, DMSO) δ 13.53 (d, J=193.9 Hz, 1H), 8.26 (s, 1H), 7.77 (s, 1H), 7.49 (s, 1H), 7.43 (s, 1H), 4.61-4.54 (m, 1H), 4.20 (s, 3H), 4.17 (s, 1H), 4.05 (dd, J=10.5, 1.3 Hz, 1H), 3.83 (d, J=11.4 Hz, 1H), 3.76-3.71 (m, 1H), 3.59 (dd, J=12.4, 10.8 Hz, 1H), 3.29-3.25 (m, 1H), 1.27 (d, J=6.6 Hz, 3H).

### Example 70

### Synthesis of (R)-4-(7-(1,4-dimethyl-1H-pyrazol-5-yl)-3-(1H-pyrazol-5-yl)isothiazolo[4,5-b]pyridin-5-yl)-3-methylmorpholine

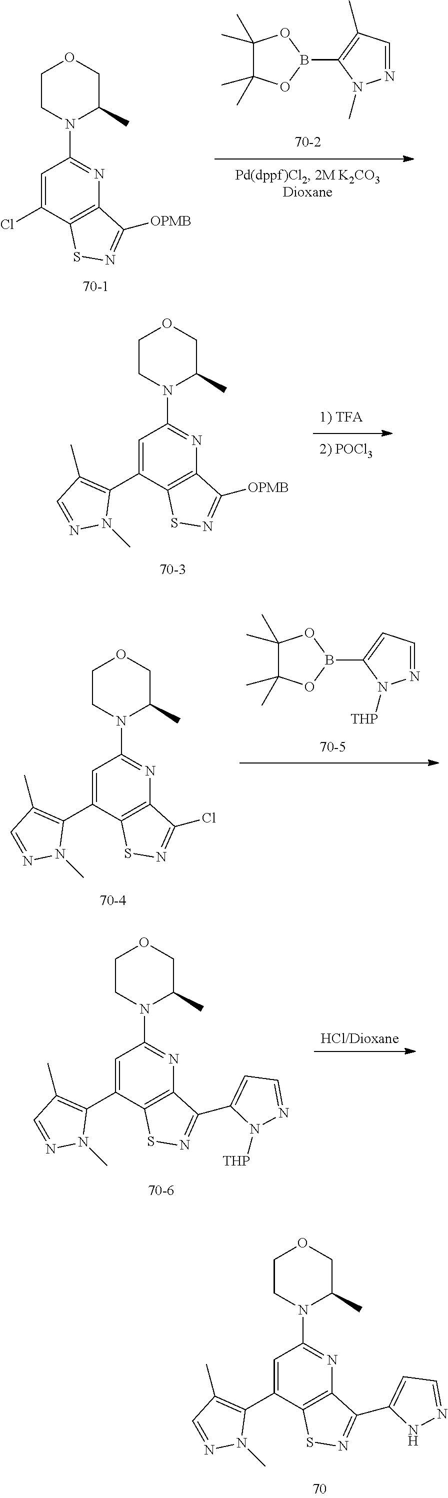

### Step 1. (R)-4-(7-(1,4-dimethyl-1H-pyrazol-5-yl)-3-((4-methoxybenzyl)oxy)isothiazolo[4,5-b]pyridin-5-yl)-3-methylmorpholine

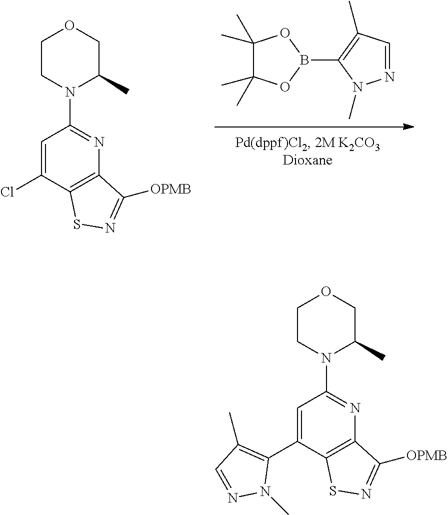

To a solution of (R)-4-(7-chloro-3-((4-methoxybenzyl)oxy)isothiazolo[4,5-b]pyridin-5-yl)-3-methylmorpholine (500 mg, 1.232 mmol) in dioxane (20 mL) was added 1,4-dimethyl-5-(4,4,5,5-tetramethyl-1,3,2-dioxaborolan-2-yl)-1H-pyrazole (547.17 mg, 2.464 mmol), K₂CO₃ (1.848 mL, 3.695 mmol) and Pd(dppf)Cl₂ (90.13 mg, 0.123 mmol), and the reaction was stirred at 100° C. for 4 hr under nitrogen atmosphere. LC-MS showed the reaction was complete. The reaction was diluted with EA (20 mL) and water (20 mL). The organic layer was separated, washed with further saturated NaCl solution, dried over anhydrous Na₂SO₄, filtered and concentrated in vacuo. The residue was purified column chromatography on silica gel (PE:EA=3:1, V/V) to afford the desired product (520 mg, 1.117 mmol, 90.67%). LC/MS (ESI) m/z: 466 (M+H)⁺.

### Step 2. (R)-4-(3-chloro-7-(1,4-dimethyl-1H-pyrazol-5-yl)isothiazolo[4,5-b]pyridin-5-yl)-3-methylmorpholine)

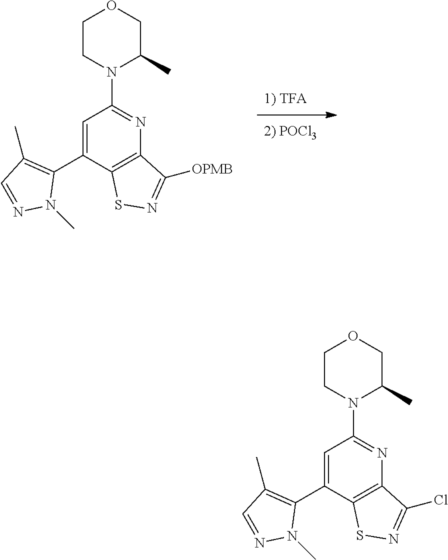

To a solution of (R)-4-(7-(1,4-dimethyl-1H-pyrazol-5-yl)-3-((4-methoxybenzyl)oxy)isothiazolo[4,5-b]pyridin-5-yl)-3-methylmorpholine (520 mg, 1.117 mmol) in TFA (10 mL). The mixture was stirred at 70° C. for 1 h. LC-MS showed the reaction was complete. The reaction mixture was concentrated under vacuo. The residue was dissolved in Toluene (30 mL) and DIEA Ethyldiisopropylamine (0.738 mL, 4.468 mmol) and POCl₃ (0.416 mL, 4.468 mmol) was added to the mixture. Then the reaction was stirred at 120° C. for 3 hr. LC-MS showed the reaction was complete. The mixture was concentrated in vacuo and the residue was purified by column chromatography on silica gel (PE:EA=2:1, V/V) to afford the desired product (130 mg, 0.357 mmol, 31.99%). LC/MS (ESI) m/z: 364 (M+H)⁺.

### Step 3. (3R)-4-(7-(1,4-dimethyl-1H-pyrazol-5-yl)-3-(1-(tetrahydro-2H-pyran-2-yl)-1H-pyrazol-5-yl)isothiazolo[4,5-b]pyridin-5-yl)-3-methylmorpholine

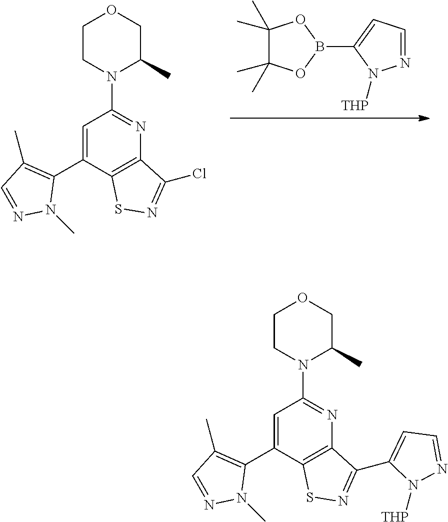

To a solution of (R)-4-(3-chloro-7-(1,4-dimethyl-1H-pyrazol-5-yl)isothiazolo[4,5-b]pyridin-5-yl)-3-methylmorpholine (60 mg, 0.165 mmol) in dioxane (2 mL) was added 1-(tetrahydro-2H-pyran-2-yl)-5-(4,4,5,5-tetramethyl-1,3,2-dioxaborolan-2-yl)-1H-pyrazole (91.73 mg, 0.330 mmol), K₂CO₃ (0.247 mL, 0.495 mmol) and Pd(PPh₃)₄ (19.05 mg, 0.016 mmol), and the reaction was stirred at 100° C. overnight under nitrogen atmosphere. LC-MS showed the reaction was complete. The reaction was diluted with EA (10 mL) and water (10 mL). The organic layer was separated, washed with further saturated NaCl solution, dried over anhydrous Na₂SO₄, filtered and concentrated in vacuo. The residue was purified Pre-TLC (DCM:MeOH=30:1, V/V) to afford the desired product (35 mg, 0.073 mmol, 44.26%). LC/MS (ESI) m/z: 480 (M+H)⁺ 496 (M+H)⁺.

### Step 4. (R)-4-(7-(1,4-dimethyl-1H-pyrazol-5-yl)-3-(1H-pyrazol-5-yl)isothiazolo[4,5-b]pyridin-5-yl)-3-methylmorpholine

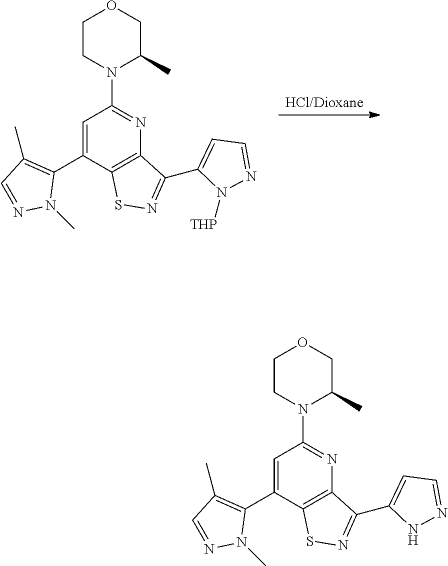

A solution of (3R)-4-(7-(1,4-dimethyl-1H-pyrazol-5-yl)-3-(1-(tetrahydro-2H-pyran-2-yl)-1H-pyrazol-5-yl)isothiazolo[4,5-b]pyridin-5-yl)-3-methylmorpholine (35 mg, 0.073 mmol) in HCl/Dioxane (4M) (2 mL) was stirred at room temperature for 1 hr. The reaction mixture was concentrated in vacuo. The residue was purified by Pre-HPLC (C₁₈, 10-95%, MeOH in H₂O with 0.1% HCOOH) to afford the desired product (10 mg, 0.025 mmol, 34.65%). LC/MS (ESI) m/z: 396 (M+H)⁺. 1H NMR (400 MHz, DMSO-d6) δ 7.72 (m, 1H), 7.48 (s, 1H), 7.44 (d, J=1.9 Hz, 1H), 7.36 (s, 1H), 4.57 (d, J=5.8 Hz, 1H), 4.21 (d, J=12.6 Hz, 1H), 4.04 (d, J=8.6 Hz, 1H), 3.81 (d, J=11.3 Hz, 1H), 3.75 (s, 3H), 3.71 (d, J=2.8 Hz, 1H), 3.62-3.55 (m, 1H), 3.27 (d, J=12.7 Hz, 1H), 1.98 (s, 3H), 1.26 (d, J=6.6 Hz, 3H).

### Example 71

### Synthesis of (R)-4-(7-(1,4-dimethyl-1H-pyrazol-5-yl)-3-(3-methyl-1H-pyrazol-5-yl)isothiazolo[4,5-b]pyridin-5-yl)-3-methylmorpholine

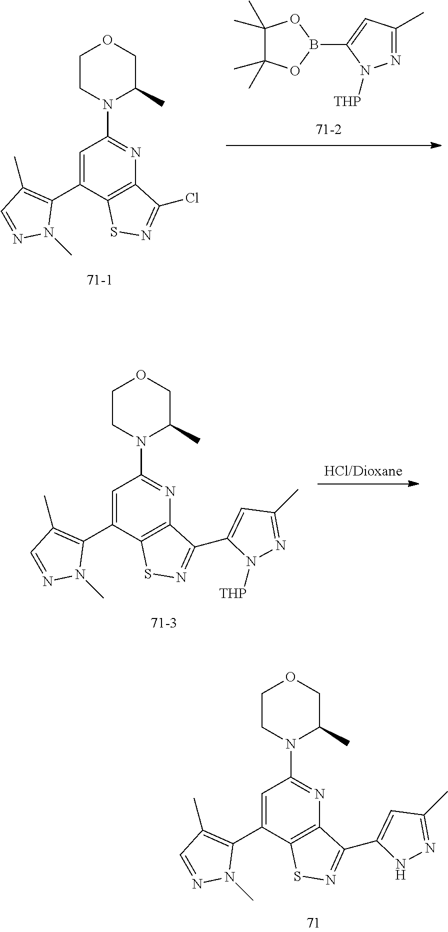

### Step 1. (3R)-4-(7-(1,4-dimethyl-1H-pyrazol-5-yl)-3-(3-methyl-1-(tetrahydro-2H-pyran-2-yl)-1H-pyrazol-5-yl)isothiazolo[4,5-b]pyridin-5-yl)-3-methylmorpholine

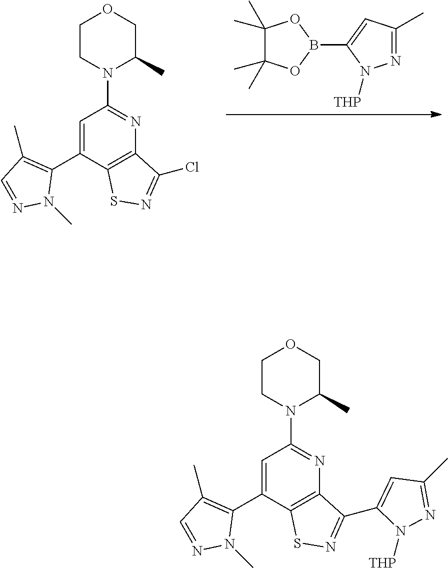

To a solution of (R)-4-(3-chloro-7-(1,4-dimethyl-1H-pyrazol-5-yl)isothiazolo[4,5-b]pyridin-5-yl)-3-methylmorpholine (60 mg, 0.165 mmol) in dioxane (2 mL) was added 3-methyl-1-(tetrahydro-2H-pyran-2-yl)-5-(4,4,5,5-tetramethyl-1,3,2-dioxaborolan-2-yl)-1H-pyrazole (144.54 mg, 0.495 mmol), K₂CO₃ (68.37 mg, 0.495 mmol) and Pd(PPh₃)₄ (19.05 mg, 0.016 mmol), and the reaction was stirred at 100° C. overnight under nitrogen atmosphere. LC-MS showed the reaction was complete. The reaction was diluted with EA (10 mL) and water (10 mL). The organic layer was separated, washed with further saturated NaCl solution, dried over anhydrous Na₂SO₄, filtered and concentrated in vacuo. The residue was purified Pre-TLC (DCM:MeOH=30:1, V/V) to afford the desired product (35 mg, 0.071 mmol, 43.00%). LC/MS (ESI) m/z: 494 (M+H)⁺ 410 (M+H)⁺.

### Step 2. (R)-4-(7-(1,4-dimethyl-1H-pyrazol-5-yl)-3-(3-methyl-1H-pyrazol-5-yl)isothiazolo[4,5-b]pyridin-5-yl)-3-methylmorpholine

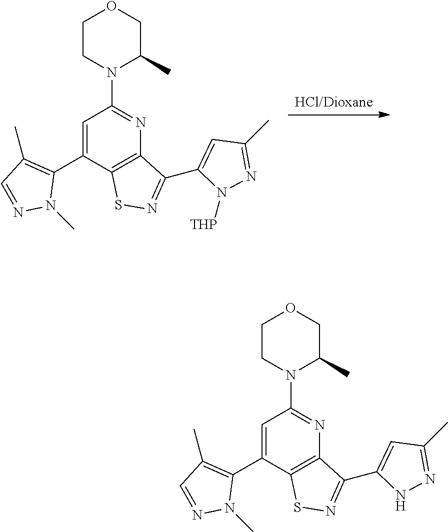

A solution of (3R)-4-(7-(1,4-dimethyl-1H-pyrazol-5-yl)-3-(3-methyl-1-(tetrahydro-2H-pyran-2-yl)-1H-pyrazol-5-yl)isothiazolo[4,5-b]pyridin-5-yl)-3-methylmorpholine (35 mg, 0.071 mmol) in HCl/Dioxane (4M) (2 mL) was stirred at room temperature for 1 hr. The reaction mixture was concentrated in vacuo. The residue was purified by Pre-HPLC (C₁₈, 10-95%, MeOH in H₂O with 0.1% HCOOH) to afford the desired product (15 mg, 0.037 mmol, 51.66%). LC/MS (ESI) m/z: 410 (M+H)⁺. ¹H NMR (400 MHz, DMSO) δ 7.48 (s, 1H), 7.35 (s, 1H), 7.16 (s, 1H), 4.55 (d, J=5.8 Hz, 1H), 4.21 (d, J=12.2 Hz, 1H), 4.04 (d, J=8.0 Hz, 1H), 3.81 (d, J=11.5 Hz, 1H), 3.73 (m, 4H), 3.60 (m, 1H), 3.29-3.23 (m, 1H), 2.33 (s, 3H), 1.98 (s, 3H), 1.26 (d, J=6.6 Hz, 3H).

### Example 72

### Synthesis of (R)-4-(7-(3,5-dimethylisoxazol-4-yl)-3-(1H-pyrazol-5-yl)isothiazolo[4,5-b]pyridin-5-yl)-3-methylmorpholine

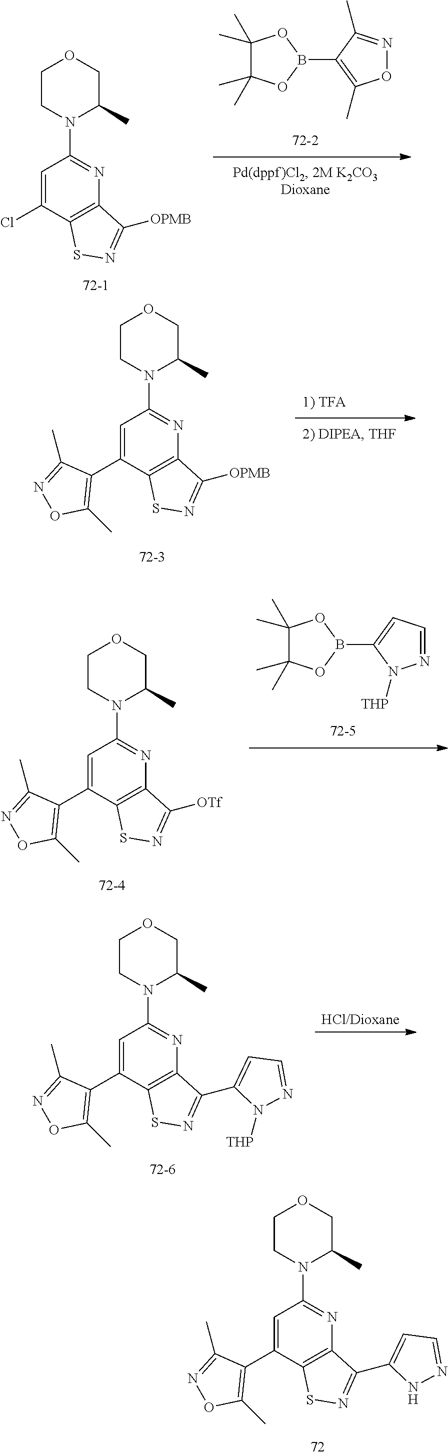

### Step 1. (R)-4-(7-(3,5-dimethylisoxazol-4-yl)-3-((4-methoxybenzyl)oxy)isothiazolo[4,5-b]pyridin-5-yl)-3-methylmorpholine

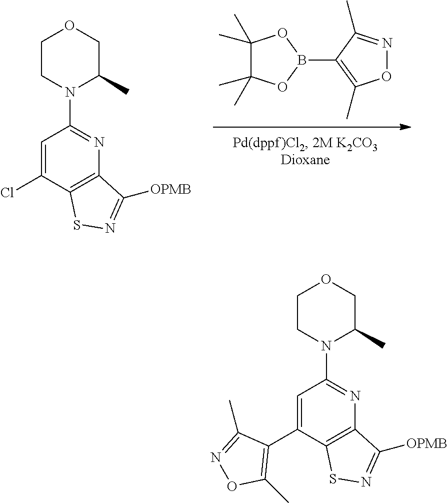

To a solution of (R)-4-(7-chloro-3-((4-methoxybenzyl)oxy)isothiazolo[4,5-b]pyridin-5-yl)-3-methylmorpholine (350 mg, 0.862 mmol) in dioxan e(13 mL) was added 3,5-dimethyl-4-(4,4,5,5-tetramethyl-1,3,2-dioxaborolan-2-yl)isoxazole (121.53 mg, 0.862 mmol), K₂CO₃ (1.293 mL, 2.587 mmol) and Pd(dppf)Cl₂ (63.09 mg, 0.086 mmol), and the reaction was stirred at 100° C. overnight under nitrogen atmosphere. LC-MS showed the reaction was complete. The reaction was diluted with EA (20 mL) and water (20 mL). The organic layer was separated, washed with further saturated NaCl solution, dried over anhydrous Na₂SO₄, filtered and concentrated in vacuo. The residue was purified column chromatography on silica gel (PE:EA=5:1, V/V) to afford the desired product (220 mg, 0.472 mmol, 54.69%). LC/MS (ESI) m/z: 467 (M+H)⁺.

### Step 2. (R)-4-(3-chloro-7-(3,5-dimethylisoxazol-4-yl)isothiazolo[4,5-b]pyridin-5-yl)-3-methylmorpholine

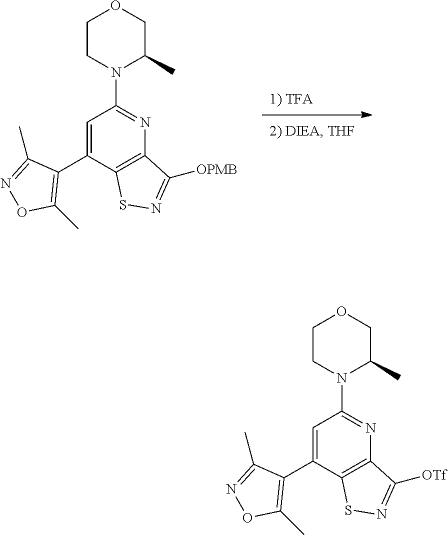

To a solution of (R)-4-(7-(3,5-dimethylisoxazol-4-yl)-3-((4-methoxybenzyl)oxy)isothiazolo[4,5-b]pyridin-5-yl)-3-methylmorpholine (220 mg, 0.472 mmol) in TFA (5 mL). The mixture was stirred at 70° C. for 1 h. LC-MS showed the reaction was complete. The reaction mixture was concentrated under vacuo. The residue was dissolved in THF (10 mL) DIEA (0.390 mL, 2.358 mmol) and N-Phenyl-bis(trifluoromethanesulfonimide) (505.37 mg, 1.415 mmol) was added to the mixture. Then the reaction was stirred at 70′C for 2 hr. LC-MS showed the reaction was complete. The mixture was concentrated in vacuo and the residue was purified by column chromatography on silica gel (PE:EA=4:1, V/V) to afford the desired product (170 mg, 0.355 mmol, 75.35%). LC/MS (ESI) m/z: 478 (M+H)⁺.

### Step 3. (R)-7-(3,5-dimethylisoxazol-4-yl)-5-(3-methylmorpholino)isothiazolo[4,5-b]pyridin-3-yl trifluoromethanesulfonate

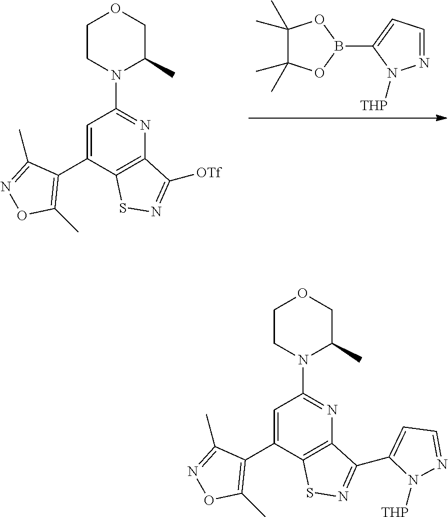

To a solution of (R)-7-(3,5-dimethylisoxazol-4-yl)-5-(3-methylmorpholino)isothiazolo[4,5-b]pyridin-3-yl trifluoromethanesulfonate (85 mg, 0.178 mmol) in DME (3 mL) was added 1-(tetrahydro-2H-pyran-2-yl)-5-(4,4,5,5-tetramethyl-1,3,2-dioxaborolan-2-yl)-1H-pyrazole (98.83 mg, 0.355 mmol), K₂CO (0.266 mL, 0.533 mmol) and Pd(dppf)Cl₂ (13.00 mg, 0.018 mmol), and the reaction was stirred at 100° C. 4 hr under nitrogen atmosphere. LC-MS showed the reaction was complete. The reaction was diluted with EA (10 mL) and water (10 mL). The organic layer was separated, washed with further saturated NaCl solution, dried over anhydrous Na₂SO₄, filtered and concentrated in vacuo. The residue was purified Pre-TLC (DCM:MeOH=30:1, V/V) to afford the desired product (30 mg, 0.062 mmol, 35.14%). LC/MS (ESI) m/z: 480 (M+H)⁺ 396 (M+H)⁺.

### Step 4. (R)-4-(7-(3,5-dimethylisoxazol-4-yl)-3-(1H-pyrazol-5-yl)isothiazolo[4,5-b]pyridin-5-yl)-3-methylmorpholine

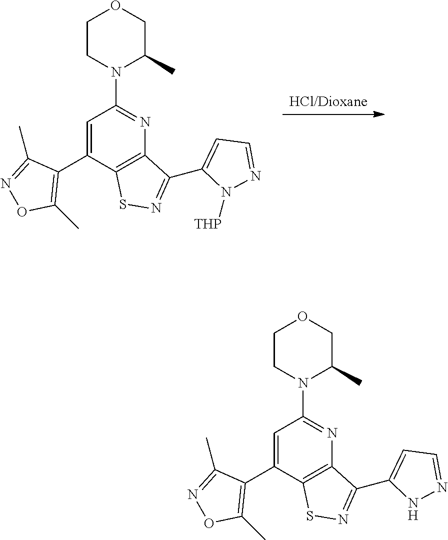

A solution of (3R)-4-(7-(3,5-dimethylisoxazol-4-yl)-3-(1-(tetrahydro-2H-pyran-2-yl)-1H-pyrazol-5-yl)isothiazolo[4,5-b]pyridin-5-yl)-3-methylmorpholine (30 mg, 0.062 mmol) in HCl/Dioxane (4M) (2 mL) was stirred at room temperature for 1 hr. The reaction mixture was concentrated in vacuo. The residue was purified by Pre-HPLC (C₁₈, 10-95%, MeOH in H₂O with 0.1% HCOOH) to afford the desired product (6 mg, 0.015 mmol, 24.24%). LC/MS (ESI) m/z: 396 (M+H)⁺. 1H NMR (400 MHz, DMSO) δ 7.78 (s, 1H), 7.43 (d, J=1.5 Hz, 1H), 7.29 (s, 1H), 4.55 (d, J=6.1 Hz, 1H), 4.18 (d, J=13.6 Hz, 1H), 4.04 (d, J=8.6 Hz, 1H), 3.81 (d, J=11.2 Hz, 1H), 3.72 (d, J=11.3 Hz, 1H), 3.57 m, 1H), 3.27 (m, 1H), 2.43 (s, 3H), 2.24 (s, 3H), 1.25 (d, J=6.7 Hz, 3H).

### Example 73

### Synthesis of (R)-4-(7-(3,5-dimethylisoxazol-4-yl)-3-(1H-pyrazol-5-yl)isothiazolo[4,5-b]pyridin-5-yl)-3-methylmorpholine

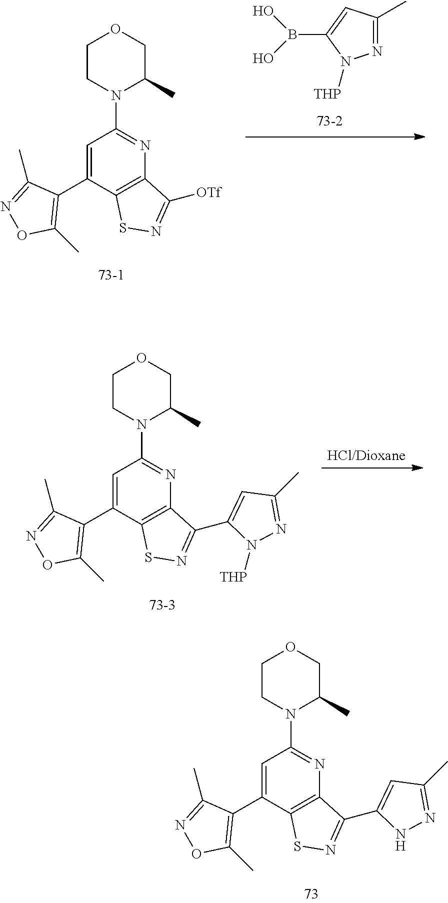

### Step 1. (3R)-4-(7-(3,5-dimethylisoxazol-4-yl)-3-(3-methyl-1-(tetrahydro-2H-pyran-2-yl)-1H-pyrazol-5-yl)isothiazolo[4,5-b]pyridin-5-yl)-3-methylmorpholine

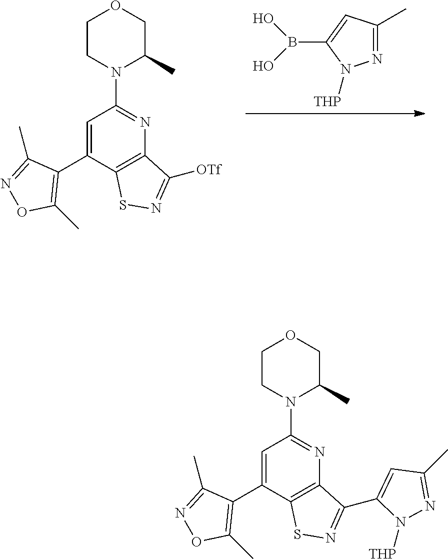

To a solution of (R)-7-(3,5-dimethylisoxazol-4-yl)-5-(3-methylmorpholino)isothiazolo[4,5-b]pyridin-3-yl trifluoromethanesulfonate (85 mg, 0.178 mmol) in DME (3 mL) was added 3-methyl-1-(tetrahydro-2H-pyran-2-yl)-5-(4,4,5,5-tetramethyl-1,3,2-dioxaborolan-2-yl)-1H-pyrazole (74.77 mg, 0.356 mmol), K₂CO₃ (0.266 mL, 0.533 mmol) and Pd(dppf)Cl₂ (13.00 mg, 0.018 mmol), and the reaction was stirred at 100° C. for 4 hr under nitrogen atmosphere. LC-MS showed the reaction was complete. The reaction was diluted with EA (10 mL) and water (10 mL). The organic layer was separated, washed with further saturated NaCl solution, dried over anhydrous Na₂SO₄, filtered and concentrated in vacuo. The residue was purified Pre-TLC (DCM:MeOH=30:1, V/V) to afford the desired product (20 mg, 0.040 mmol, 22.72%). LC/MS (ESI) m/z: 494 (M+H)⁺ 410 (M+H)⁺.

### Step 2. (R)-4-(7-(3,5-dimethylisoxazol-4-yl)-3-(3-methyl-1H-pyrazol-5-yl)isothiazolo[4,5-b]pyridin-5-yl)-3-methylmorpholine e

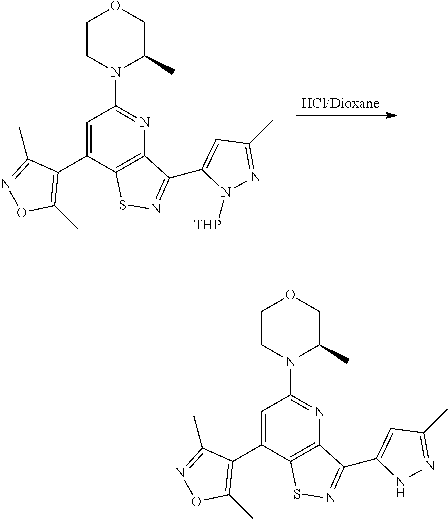

A solution of (3R)-4-(7-(3,5-dimethylisoxazol-4-yl)-3-(3-methyl-1-(tetrahydro-2H-pyran-2-yl)-1H-pyrazol-5-yl)isothiazolo[4,5-b]pyridin-5-yl)-3-methylmorpholine (20 mg, 0.040 mmol) in HCl/Dioxane (4M) (2 mL) was stirred at room temperature for 1 hr. The reaction mixture was concentrated in vacuo. The residue was purified by Pre-HPLC (C₁₈, 10-95%, MeOH in H₂O with 0.1% HCOOH) to afford the desired product (6 mg, 0.015 mmol, 36.14%). LC/MS (ESI) m/z: 411 (M+H)⁺. 1H NMR (400 MHz, DMSO) δ 7.27 (s, 1H), 7.15 (s, 1H), 4.53 (d, J=5.9 Hz, 1H), 4.18 (d, J=12.4 Hz, 1H), 4.04 (d, J=8.6 Hz, 1H), 3.81 (d, J=11.4 Hz, 1H), 3.73 (d, J=11.4 Hz, 1H), 3.58 (t, J=10.3 Hz, 1H), 3.23 (s, 1H), 2.42 (s, 3H), 2.32 (s, 3H), 2.24 (s, 3H), 1.25 (d, J=6.6 Hz, 3H).

### Example 74

### Synthesis of (R)-2-(3-(3-methyl-1H-pyrazol-5-yl)-5-(3-methylmorpholino)isothiazolo[4,5-b]pyridin-7-yl)propan-2-ol

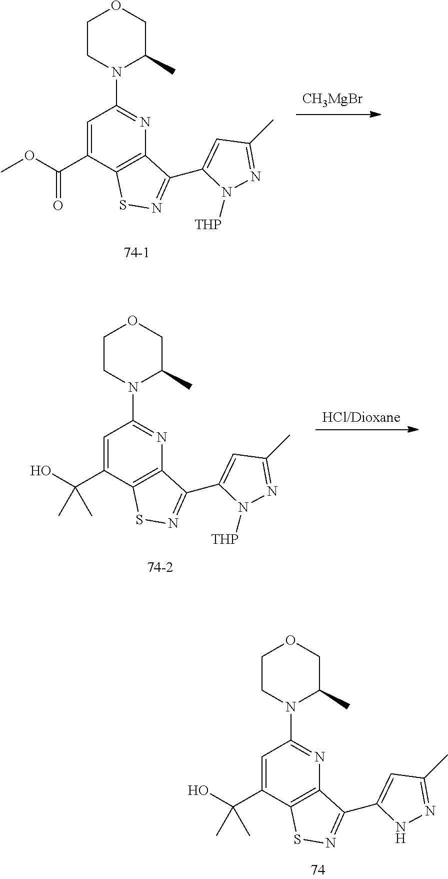

### Step 1. 2-(3-(3-methyl-1-(tetrahydro-2H-pyran-2-yl)-1H-pyrazol-5-yl)-5-((R)-3-methylmorpholino)isothiazolo[4,5-b]pyridin-7-yl)propan-2-ol

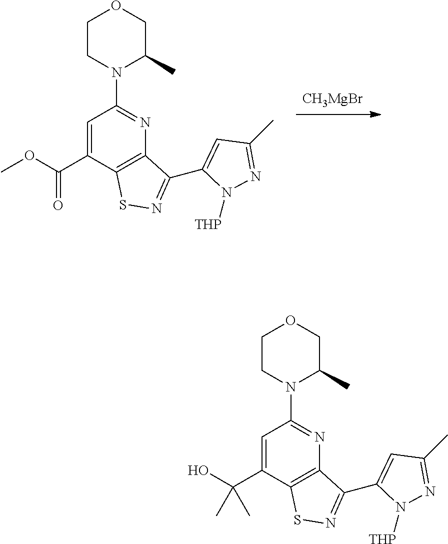

To solution of methyl 3-(3-methyl-1-(tetrahydro-2H-pyran-2-yl)-1H-pyrazol-5-yl)-5-((R)-3-methylmorpholino)isothiazolo[4,5-b]pyridine-7-carboxylate (70 mg, 0.153 mmol) in THF (5 mL) were added Methyl magnesium bromide (in ethyl ether) (0.153 mL, 0.459 mmol) drop wise at 0° C. After stirring at 0° C. for 30 min, the mixture was warmed to room temperature and stirred for another 1 hr. The reaction was quenched with saturated NH₄Cl solution and diluted with EA. The organic layer was separated and concentrated in vacuo. The residue was purified Prep-TLC (PE:EA=1:1, V/V) to give desired product (35 mg, 0.076 mmol, 49.99%). LC/MS (ESI) (m/z): 458 (M+H)⁺.

### Step 2. (R)-2-(3-(3-methyl-1H-pyrazol-5-yl)-5-(3-methylmorpholino)isothiazolo[4,5-b]pyridin-7-yl)propan-2-ol

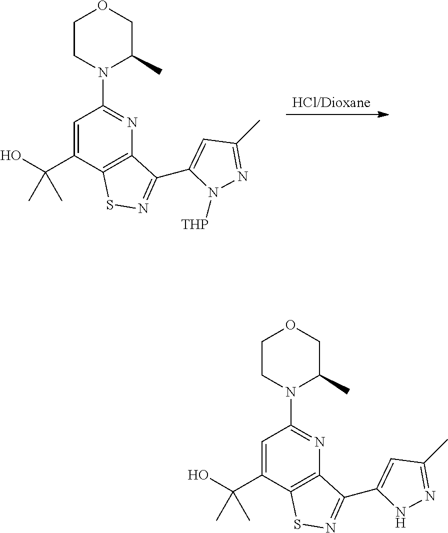

A solution of 2-(3-(3-methyl-1-(tetrahydro-2H-pyran-2-yl)-1H-pyrazol-5-yl)-5-((R)-3-methylmorpholino)isothiazolo[4,5-b]pyridin-7-yl)propan-2-ol (30 mg, 0.066 mmol) in HCl/Dioxane (4M) (2 mL) was stirred at room temperature for 1 hr. The reaction mixture was concentrated in vacuo. The residue was purified by Pre-HPLC (C₁₈, 10-95%, MeOH in H₂O with 0.1% HCOOH) to afford the desired product (17 mg, 0.046 mmol, 69.42%). LC/MS (ESI) m/z: 374 (M+H)⁺. ¹H NMR (400 MHz, DMSO) δ 12.95 (d, J=103.8 Hz, 1H), 7.09 (s, 1H), 7.02 (s, 1H), 6.06 (s, 1H), 4.53 (s, 1H), 4.09 (d, J=12.9 Hz, 1H), 4.02 (d, J=9.1 Hz, 1H), 3.81 (d, J=11.3 Hz, 1H), 3.72 (d, J=11.1 Hz, 1H), 3.57 (t, J=10.8 Hz, 1H), 3.22 (t, J=11.1 Hz, 1H), 2.29 (s, 3H), 1.56 (s, 6H), 1.21 (d, J=6.6 Hz, 3H).

### Example 75

### Synthesis of (R)-4-(7-(cyclopropylsulfonyl)-3-(1H-pyrazol-5-yl)isothiazolo[4,5-b]pyridin-5-yl)-3-methylmorpholine

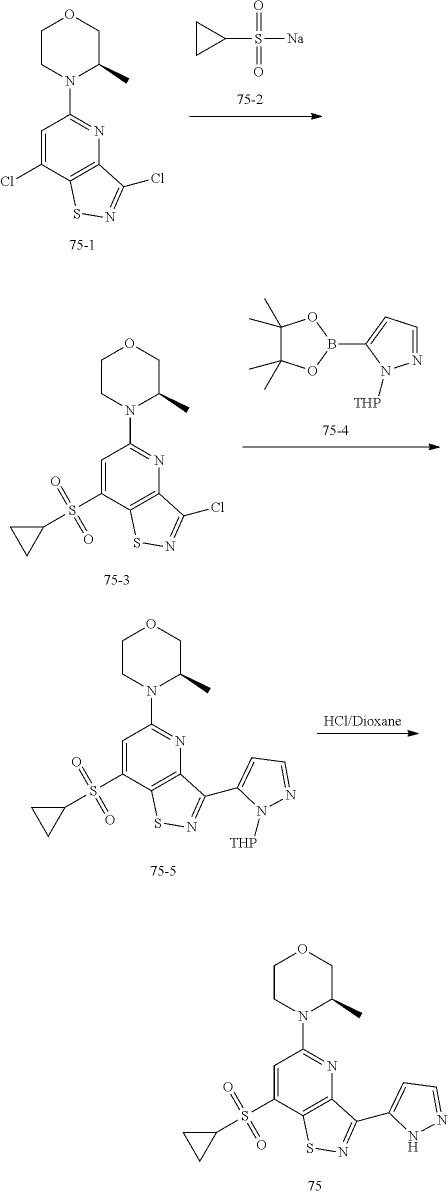

### Step 1. (R)-4-(3-chloro-7-(cyclopropylsulfonyl)isothiazolo[4,5-b]pyridin-5-yl)-3-methylmorpholine

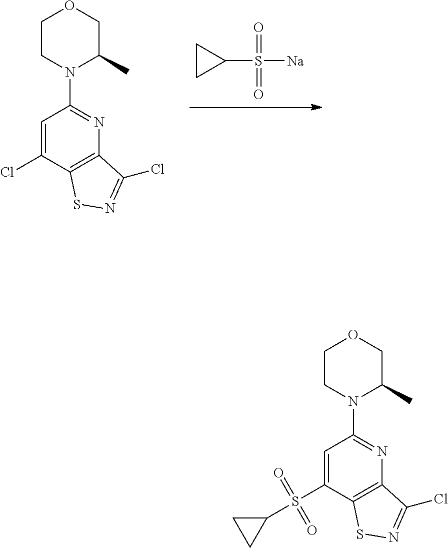

To a solution of (R)-4-(3,7-dichloroisothiazolo[4,5-b]pyridin-5-yl)-3-methylmorpholine (150 mg, 0.493 mmol) in DMF (5 mL) was added sodium cyclopropanesulfinate (94.77 mg, 0.740 mmol) and Cs₂CO₃ (321.32 mg, 0.986 mmol), and the reaction was stirred at 70° C. overnight. LC-MS showed the reaction was complete. The reaction was diluted with EA (10 mL) and water (10 mL). The organic layer was separated, washed with saturated NaCl, dried over anhydrous Na₂SO₄, filtered and concentrated in vacuo. The residue was purified by Prep-TLC (PE:EA=2:1, V/V) to afford the desired product (70 mg, 0.187 mmol, 37.97%). LC/MS (ESI) m/z: 374 (M+H)⁺. ¹H NMR (400 MHz, CDCl3) δ 7.28 (s, 1H), 4.46 (d, J=6.7 Hz, 1H), 4.17 (dd, J=13.4, 2.5 Hz, 1H), 4.10 (dd, J=11.5, 3.7 Hz, 1H), 3.88 (d, J=11.5 Hz, 1H), 3.81 (dd, J=11.6, 3.0 Hz, 1H), 3.66 (td, J=11.9, 3.0 Hz, 1H), 3.41 (td, J=12.7, 3.9 Hz, 1H), 2.61-2.52 (m, 1H), 1.47 (dd, J=4.6, 2.2 Hz, 2H), 1.36 (d, J=6.8 Hz, 3H), 1.12 (dd, J=7.9, 2.0 Hz, 2H).

### Step 2. (3R)-4-(7-(cyclopropylsulfonyl)-3-(1-(tetrahydro-2H-pyran-2-yl)-1H-pyrazol-5-yl)isothiazolo[4,5-b]pyridin-5-yl)-3-methylmorpholine

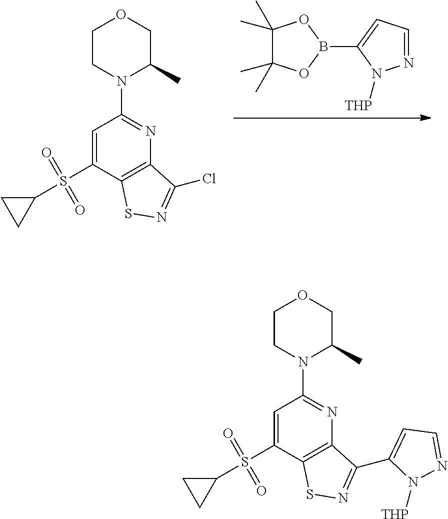

To a solution of (R)-4-(3-chloro-7-(cyclopropylsulfonyl)isothiazolo[4,5-b]pyridin-5-yl)-3-methylmorpholine (60 mg, 0.160 mmol) in dioxane (2.5 mL) was added 1-(tetrahydro-2H-pyran-2-yl)-5-(4,4,5,5-tetramethyl-1,3,2-dioxaborolan-2-yl)-1H-pyrazole (94.36 mg, 0.481 mmol), K₂CO₃ (0.241 mL, 0.481 mmol) and Pd(dppf)Cl₂ (11.74 mg, 0.016 mmol), and the reaction was stirred at 100° C. overnight under nitrogen atmosphere. LC-MS showed the reaction was complete. The reaction was diluted with EA (10 mL) and water (10 mL). The organic layer was separated, washed with further saturated NaCl, dried over anhydrous Na₂SO₄, filtered and concentrated in vacuo. The residue was purified by Pre-TLC (DCM:MeOH=30:1, V/V) to afford the desired product (50 mg, 0.102 mmol, 63.64%). LC/MS (ESI) m/z: 489 (M+H)⁺.

### Step 3. (R)-4-(7-(cyclopropylsulfonyl)-3-(1H-pyrazol-5-yl)isothiazolo[4,5-b]pyridin-5-yl)-3-methylmorpholine

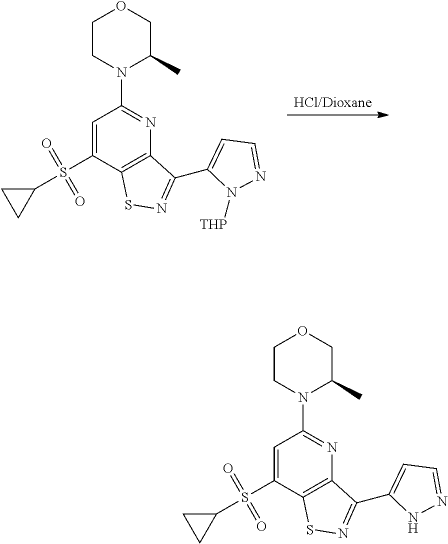

A solution of (R)-4-(7-(cyclopropylsulfonyl)-3-(1H-pyrazol-5-yl)isothiazolo[4,5-b]pyridin-5-yl)-3-methylmorpholine (50 mg, 0.102 mmol) in HCl solution (4M in dioxane, 2 mL) was stirred at room temperature for 1 hr. LC-MS showed the reaction was complete. The reaction mixture was concentrated in vacuo. The residue was purified by prep-HPLC (C₁₈, 10-95%, MeOH in H₂O with 0.1% TFA) to afford the desired product (10 mg, 0.025 mmol, 24.15%). LC/MS (ESI) m/z: 406 (M+H)⁺. ¹H NMR (400 MHz, DMSO) δ 13.55 (d, J=174.5 Hz, 1H), 7.68 (s, 2H), 7.38 (s, 1H), 4.60 (s, 1H), 4.19 (d, J=12.8 Hz, 1H), 4.08-4.02 (m, 1H), 3.83 (d, J=11.4 Hz, 1H), 3.73 (dd, J=11.5, 2.8 Hz, 1H), 3.58 (dd, J=11.6, 9.1 Hz, 1H), 3.29 (s, 1H), 3.22-3.19 (m, 1H), 1.29 (d, J=3.4 Hz, 2H), 1.27 (d, J=6.7 Hz, 3H), 1.16 (dd, J=7.8, 2.3 Hz, 2H).

### Example 76

### Synthesis of (R)-2-(5-(3-methylmorpholino)-3-(1H-pyrazol-5-yl)isothiazolo[4,5-b]pyridin-7-yl)-1,2-thiazinane 1,1-dioxide

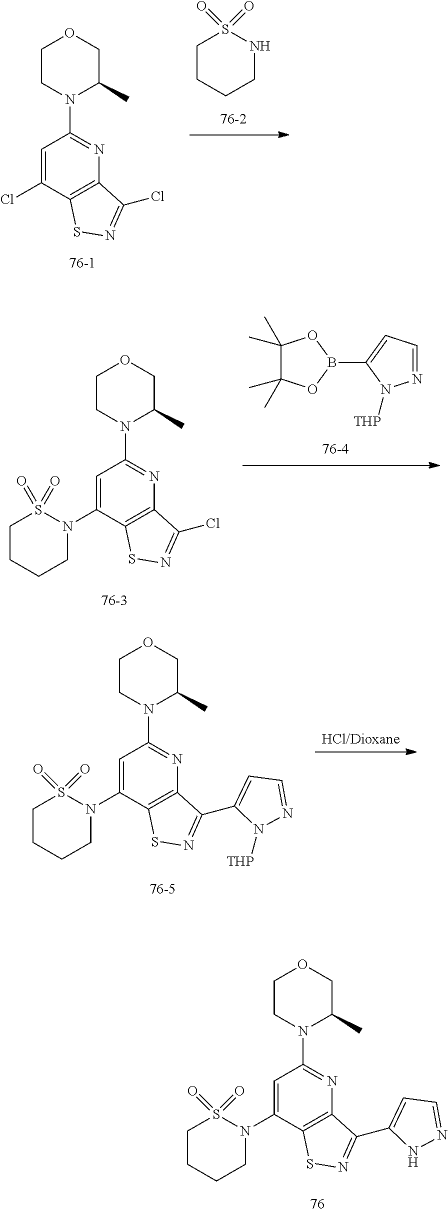

### Step 1. (R)-2-(3-chloro-5-(3-methylmorpholino)isothiazolo[4,5-b]pyridin-7-yl)-1,2-thiazinane 1,1-dioxide

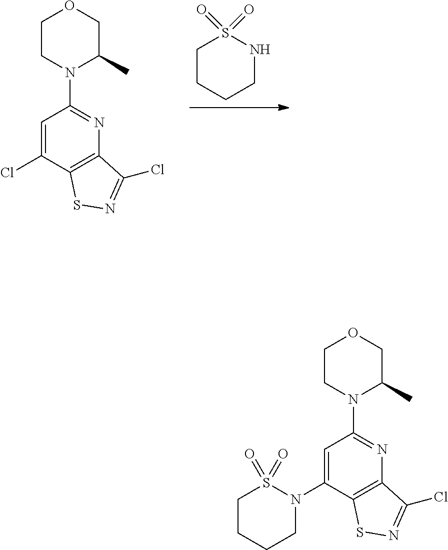 [SMILES: C[C@@H]1COCCN1c1cc(Cl)c2snc(Cl)c2n1 ; O=S1(=O)CCCCN1 ; C[C@@H]1COCCN1c1cc(N2CCCCS2(=O)=O)c2snc(Cl)c2n1]

To a solution of (R)-4-(3,7-dichloroisothiazolo[4,5-b]pyridin-5-yl)-3-methylmorpholine (150 mg, 0.493 mmol) in toluene (5 mL) was added 1,2-thiazinane 1,1-dioxide (99.99 mg, 0.740 mmol), Cs₂CO₃ (321.32 mg, 0.986 mmol) and Pd(OAc)₂ (11.07 mg, 0.049 mmol), and the reaction was stirred at 100° C. overnight under nitrogen atmosphere. LC-MS showed the reaction was complete. The reaction was diluted with EA (20 mL) and water (20 mL). The organic layer was separated, washed with saturated NaCl, dried over anhydrous Na₂SO₄, filtered and concentrated in vacuo. The residue was purified by column chromatography on silica gel (PE:EA=2:1, V/V) to afford the desired product (50 mg, 0.124 mmol, 25.17%). LC/MS (ESI) m/z: 403 (M+H)⁺.

### Step 2. 2-(5-((R)-3-methylmorpholino)-3-(1-(tetrahydro-2H-pyran-2-yl)-1H-pyrazol-5-yl)isothiazolo[4,5-b]pyridin-7-yl)-1,2-thiazinane 1,1-dioxide

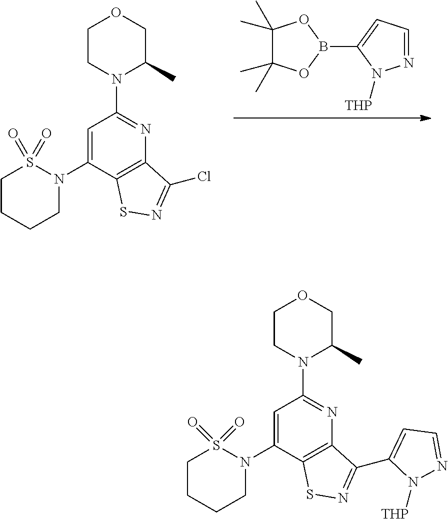

To a solution of (R)-2-(3-chloro-5-(3-methylmorpholino)isothiazolo[4,5-b]pyridin-7-yl)-1,2-thiazinane 1,1-dioxide (50 mg, 0.124 mmol) in dioxane (1.5 mL) was added 1-(tetrahydro-2H-pyran-2-yl)-5-(4,4,5,5-tetramethyl-1,3,2-dioxaborolan-2-yl)-1H-pyrazole (69.03 mg, 0.248 mmol), K₂CO₃ (0.186 mL, 0.372 mmol) and Pd(PPh₃)₄ (143.39 mg, 0.124 mmol). The reaction was stirred at 100° C. overnight under nitrogen atmosphere. LC-MS showed the reaction was complete. The reaction was diluted with EA (10 mL) and water (10 mL). The organic layer was separated, washed with saturated NaCl, dried over anhydrous Na₂SO₄, filtered and concentrated in vacuo. The residue was purified by prep-TLC (DCM:MeOH=30:1, V/V) to afford the desired product (30 mg, 0.058 mmol, 46.61%). LC/MS (ESI) m/z: 519 (M+H)⁺.

### Step 3. (R)-2-(5-(3-methylmorpholino)-3-(1H-pyrazol-5-yl)isothiazolo[4,5-b]pyridin-7-yl)-1,2-thiazinane 1,1-dioxide

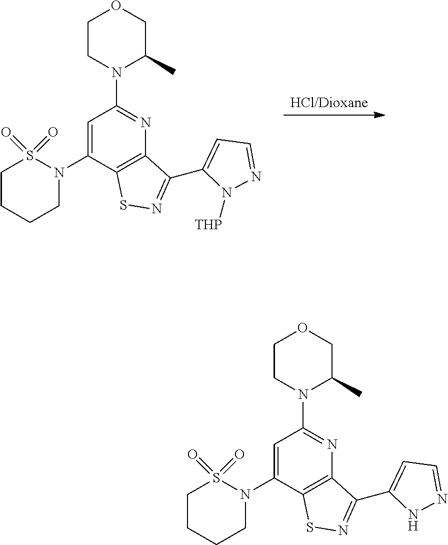

A solution of 2-(5-((R)-3-methylmorpholino)-3-(1-(tetrahydro-2H-pyran-2-yl)-1H-pyrazol-5-yl)isothiazolo[4,5-b]pyridin-7-yl)-1,2-thiazinane 1,1-dioxide (30 mg, 0.058 mmol) in HCl solution (4M in dioxane, 2 mL) was stirred at room temperature for 1 hr. LC-MS showed the reaction was complete. The reaction mixture was concentrated in vacuo. The residue was purified by Pre-HPLC (C₁₈, 10-95%, MeOH in H₂O with 0.1% TFA) to afford the desired product (10 mg, 0.023 mmol, 39.79%). LC/MS (ESI) m/z: 435 (M+H)⁺.1H NMR (400 MHz, DMSO) δ 13.46 (d, J=166.0 Hz, 1H), 7.77 (d, J=88.4 Hz, 1H), 7.35 (d, J=1.9 Hz, 1H), 7.06 (s, 1H), 4.50 (s, 1H), 4.13-3.97 (m, 2H), 3.82 (dd, J=13.8, 8.4 Hz, 3H), 3.69 (dd, J=11.4, 2.8 Hz, 1H), 3.58-3.51 (m, 1H), 3.50-3.46 (m, 2H), 3.25-3.21 (m, 1H), 2.21 (s, 2H), 1.87 (s, 2H), 1.22 (d, J=6.6 Hz, 3H)

### Example 77

### Synthesis of (R)—N-(3-chloro-1H-pyrazol-5-yl)-4-(3-methylmorpholino)-6-(1-(methylsulfonyl)cyclopropyl)pyrimidin-2-amine

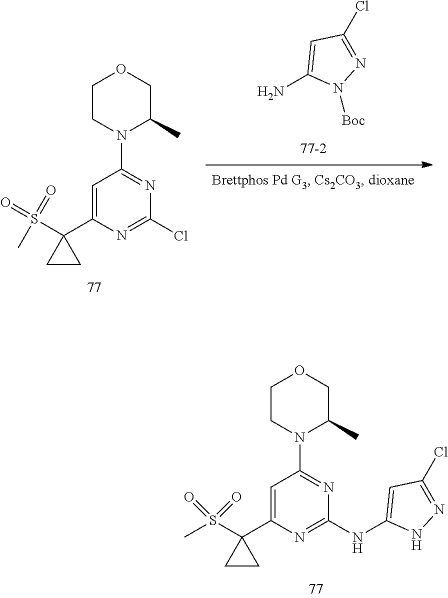 [SMILES: C[C@@H]1COCCN1c1cc(C2(S(C)(=O)=O)CC2)nc(Cl)n1 ; CC(C)(C)OC(=O)n1nc(Cl)cc1N ; C[C@@H]1COCCN1c1cc(C2(S(C)(=O)=O)CC2)nc(Nc2cc(Cl)n[nH]2)n1]

### Step 1. (R)—N-(3-chloro-1H-pyrazol-5-yl)-4-(3-methylmorpholino)-6-(1-(methylsulfonyl)cyclopropyl)pyrimidin-2-amine

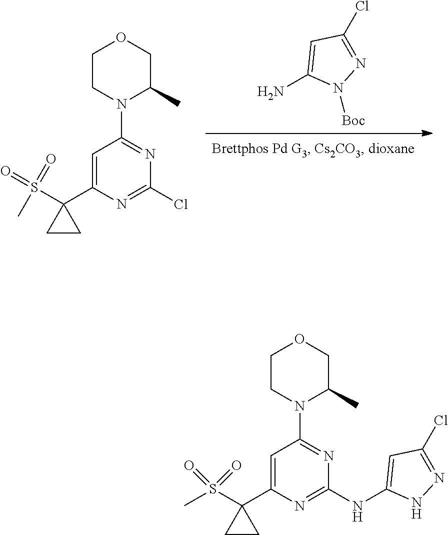 [SMILES: C[C@@H]1COCCN1c1cc(C2(S(C)(=O)=O)CC2)nc(Cl)n1 ; CC(C)(C)OC(=O)n1nc(Cl)cc1N ; C[C@@H]1COCCN1c1cc(C2(S(C)(=O)=O)CC2)nc(Nc2cc(Cl)n[nH]2)n1]

To a solution of (3R)-4-[2-chloro-6-(1-methanesulfonylcyclopropyl)pyrimidin-4-yl]-3-methylmorpholine (87 mg, 0.26 mmol) and tert-butyl 5-amino-3-chloro-1H-pyrazole-1-carboxylate (86 mg, 0.39 mmol) in dioxane (4 mL) were added BrettPhos Pd G3 (24 mg, 0.02 mmol) and Cs₂CO₃ (172 mg, 0.52 mmol). The mixture was stirred at 100° C. for 16 h under N₂ atmosphere. LC-MS showed the reaction was complete. The reaction mixture was diluted with EA (40 mL), then washed with water and brine, dried over anhydrous Na₂SO₄, filtered and concentrated. The residue was purified by prep-HPLC (C18, 10-95%, MeOH in H₂O with 0.1% HCOOH) to give the desired product (43 mg, yield: 39%). 1H NMR (400 MHz, DMSO-d6) δ 12.24 (s, 1H), 9.86 (s, 1H), 6.37 (s, 1H), 5.93 (s, 1H), 4.41 (s, 1H), 4.02 (d, J=13.7 Hz, 1H), 3.93 (dd, J=11.4, 3.4 Hz, 1H), 3.73 (d, J=11.4 Hz, 1H), 3.58 (dd, J=11.5, 2.9 Hz, 1H), 3.43 (td, J=11.9, 2.9 Hz, 1H), 3.19-3.10 (m, 4H), 1.63 (t, J=5.8 Hz, 2H), 1.51 (s, 2H), 1.19 (d, J=6.7 Hz, 3H).

### Example 78

### Synthesis of 3-[4-(1-methyl-1H-pyrazol-5-yl)-7-(1H-pyrazol-5-yl)imidazo[1,5-b]pyridazin-2-yl]-8-oxa-3-azabicyclo[3.2.1]octane

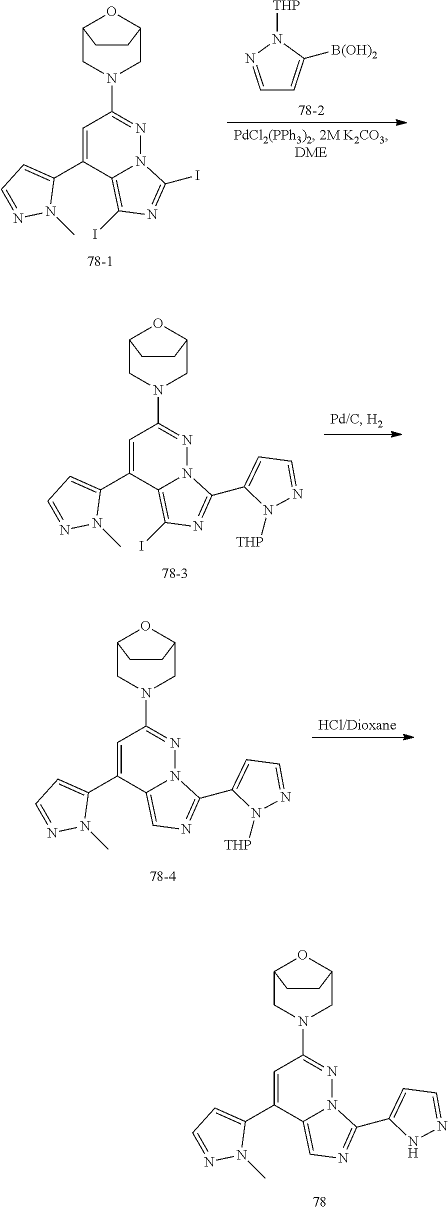

### Step 1. 3-[4-(1-methyl-1H-pyrazol-5-yl)-7-[1-(oxan-2-yl)-1H-pyrazol-5-yl]imidazo[1,5-b]pyridazin-2-yl]-8-oxa-3-azabicyclo[3.2.1]octane

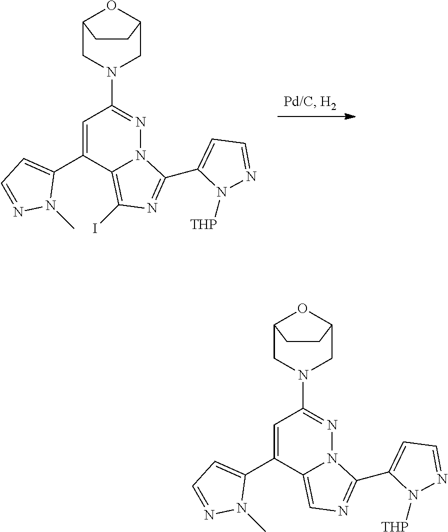

To a solution of 3-[5-iodo-4-(1-methyl-1H-pyrazol-5-yl)-7-[1-(oxan-2-yl)-1H-pyrazol-5-yl]imidazo[1,5-b]pyridazin-2-yl]-8-oxa-3-azabicyclo[3.2.1]octane (250 mg, 0.426 mmol) in MeOH (15 mL) were added Pd/C (0.044 mL, 0.426 mmol) under H₂ atmosphere, and the reaction was stirred at room temperature overnight. Then the reaction was concentrated in vacuo to afford 3-[4-(1-methyl-1H-pyrazol-5-yl)-7-[1-(oxan-2-yl)-1H-pyrazol-5-yl]imidazo[1,5-b]pyridazin-2-yl]-8-oxa-3-azabicyclo[3.2.1]octane (100 mg, 0.217 mmol, 50.93%). LC/MS (ESI) m/z: 461 (M+H)⁺

### Step 2. 3-[4-(1-methyl-1H-pyrazol-5-yl)-7-(1H-pyrazol-5-yl)imidazo[1,5-b]pyridazin-2-yl]-8-oxa-3-azabicyclo[3.2.1]octane

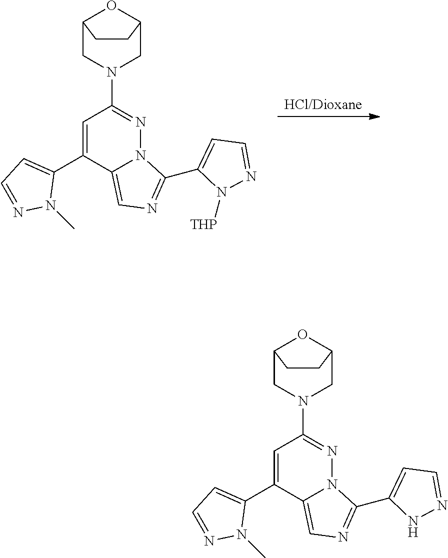

To a solution of 3-[4-(1-methyl-1H-pyrazol-5-yl)-7-[1-(oxan-2-yl)-1H-pyrazol-5-yl]imidazo[1,5-b]pyridazin-2-yl]-8-oxa-3-azabicyclo[3.2.1]octane (100 mg, 0.217 mmol) in DCM (10 mL) were added HCl/dioxane (10 mL), and the reaction was stirred at room temperature for 1 hour. The reaction mixture was extracted with Ethyl Acetate, washed with H₂O and brine, dried over Na₂SO₄, filtered and concentrated in vacuum to give the title product 3-[4-(1-methyl-1H-pyrazol-5-yl)-7-(1H-pyrazol-5-yl)imidazo[1,5-b]pyridazin-2-yl]-8-oxa-3-azabicyclo[3.2.1]octane (7 mg, 0.019 mmol, 8.56%). LC/MS (ESI) m/z:377 (M+H)⁺. ¹H NMR (400 MHz, DMSO) δ 7.73 (s, 1H), 7.66 (d, J=1.9 Hz, 1H), 7.44 (s, 1H), 7.14 (d, J=1.8 Hz, 1H), 6.94 (s, 1H), 6.80 (d, J=1.9 Hz, 1H), 4.51 (s, 2H), 3.96 (d, J=14.5 Hz, 3H), 3.93 (d, J=12.4 Hz, 2H), 3.24-3.10 (m, 2H), 1.87 (s, 4H).

### Example 79

### Synthesis of 3-[4-(1-methyl-1H-pyrazol-5-yl)-7-(3-methyl-1H-pyrazol-5-yl)imidazo[1,5-b]pyridazin-2-yl]-8-oxa-3-azabicyclo[3.2.1]octane

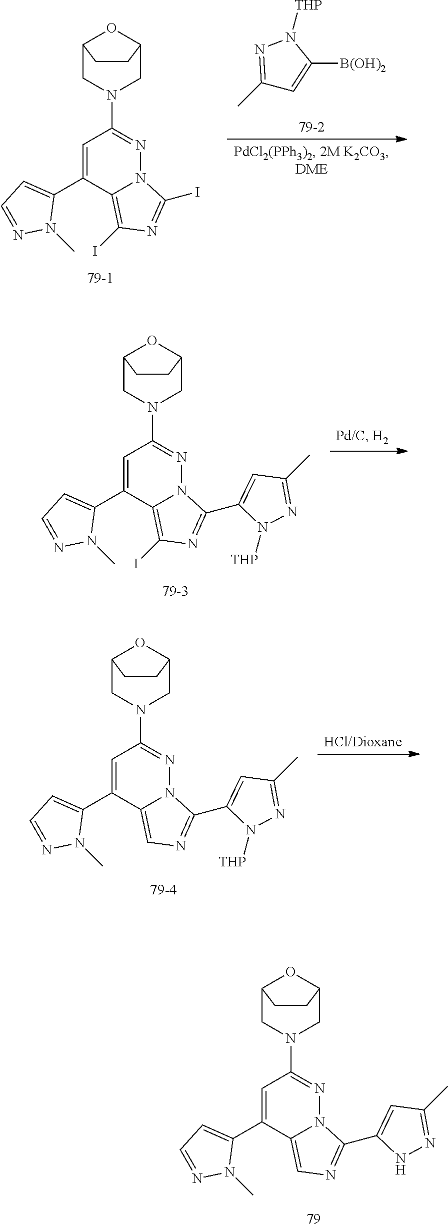

### Step 1. 3-{7-[3-methyl-1-(oxan-2-yl)-1H-pyrazol-5-yl]-4-(1-methyl-1H-pyrazol-5-yl)imidazo[1,5-b]pyridazin-2-yl}-8-oxa-3-azabicyclo[3.2.1]octane

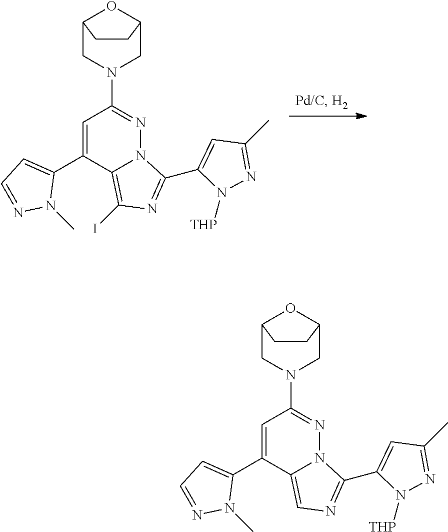

To a solution of 3-{5-iodo-7-[3-methyl-1-(oxan-2-yl)-1H-pyrazol-5-yl]-4-(1-methyl-1H-pyrazol-5-yl)imidazo[1,5-b]pyridazin-2-yl}-8-oxa-3-azabicyclo[3.2.1]octane (150 mg, 0.250 mmol) in MeOH (10 mL) were added Pd/C (0.026 mL, 0.250 mmol) at the H₂ protection, and the reaction was stirred at room temperature overnight. Then the reaction was concentrated in vacuo to afford 3-{7-[3-methyl-1-(oxan-2-yl)-1H-pyrazol-5-yl]-4-(1-methyl-1H-pyrazol-5-yl)imidazo[1,5-b]pyridazin-2-yl}-8-oxa-3-azabicyclo[3.2.1]octane (96 mg, 0.202 mmol, 80.98%). LC/MS (ESI) m/z:475 (M+H)⁺

### Step 2.3-[4-(1-methyl-1H-pyrazol-5-yl)-7-(3-methyl-1H-pyrazol-5-yl)imidazo[1,5-b]pyridazin-2-yl]-8-oxa-3-azabicyclo[3.2.1]octane

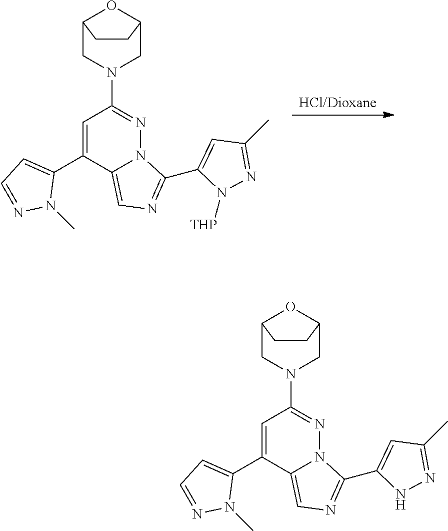

To a solution of 3-{7-[3-methyl-1-(oxan-2-yl)-1H-pyrazol-5-yl]-4-(1-methyl-1H-pyrazol-5-yl)imidazo[1,5-b]pyridazin-2-yl}-8-oxa-3-azabicyclo[3.2.1]octane (96 mg, 0.202 mmol) in DCM (6 mL) were added HCl/Dioxane (6 mL), and the reaction was stirred at room temperature for 1 hour. The reaction mixture was extracted with Ethyl Acetate, washed with H₂O and brine, dried over Na₂SO₄, filtered and concentrated in vacuum to give the title product 3-[4-(1-methyl-1H-pyrazol-5-yl)-7-(3-methyl-1H-pyrazol-5-yl)imidazo[1,5-b]pyridazin-2-yl]-8-oxa-3-azabicyclo[3.2.1]octane (10 mg, 0.026 mmol, 12.66%). LC/MS (ESI) m/z:391 (M+H)⁺.

¹H NMR (400 MHz, DMSO) δ 7.65 (d, J=1.9 Hz, 1H), 7.41 (s, 1H), 6.92 (s, 1H), 6.88 (s, 1H), 6.79 (d, J=1.9 Hz, 1H), 4.51 (s, 2H), 3.98 (s, 3H), 3.92 (d, J=12.4 Hz, 2H), 3.18 (dd, J=12.5, 2.2 Hz, 2H), 2.30 (s, 3H), 1.87 (s, 4H).

### Example 80

### Synthesis of (R)-1-(3-(3-methyl-1H-pyrazol-5-yl)-5-(3-methylmorpholino)isothiazolo[4,5-b]pyridin-7-yl)cyclopentane-1-carbonitrile

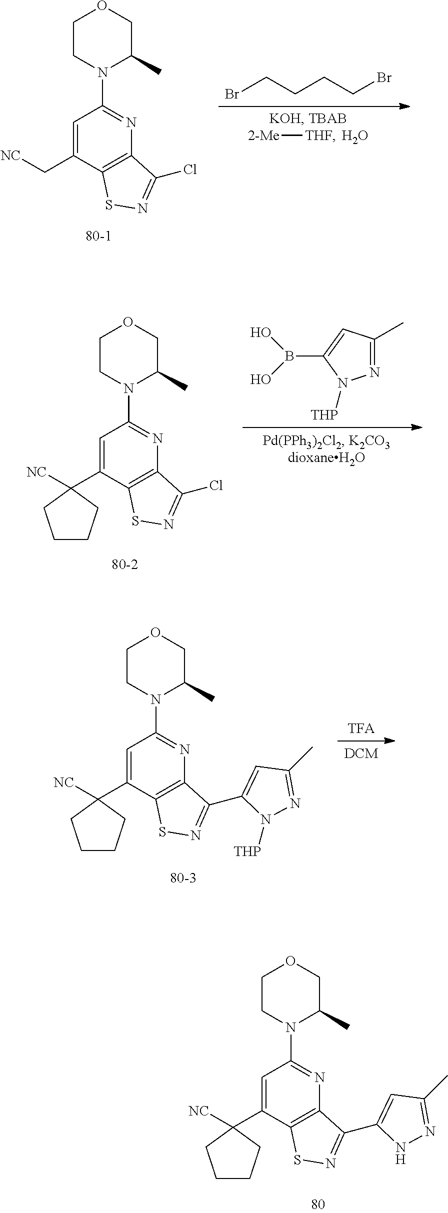

### Step 1. (R)-1-(3-chloro-5-(3-methylmorpholino)isothiazolo[4,5-b]pyridin-7-yl)cyclopentane-1-carbonitrile

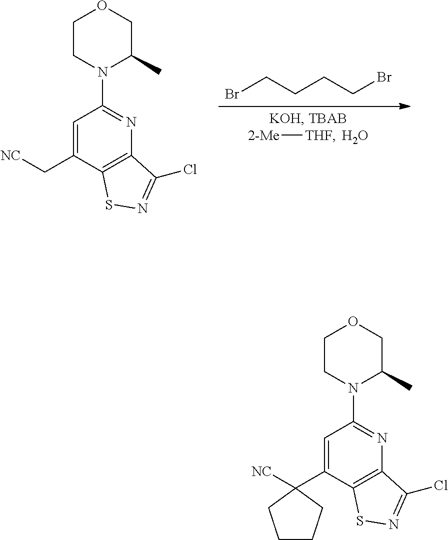

A mixture of 2-{3-chloro-5-[(3R)-3-methylmorpholin-4-yl]-[1,2]thiazolo[4,5-b]pyridin-7-yl}acetonitrile (152 mg, 0.492 mmol), 1,4-dibromobutane (0.235 mL, 1.969 mmol), KOH (552.40 mg, 9.845 mmol) and TBAB (0.031 mL, 0.098 mmol) in 2-methyltetrahydrofuran (10 mL) and water (1 mL) was stirred at 80° C. for 4 hrs under nitrogen atmosphere. The reaction mixture was diluted with water and extracted with ethyl acetate. The combined organic layer was washed with brine, dried over Na₂SO₄, filtered and concentrated in vacuo. The residue was purified on flash column chromatography (Silica, 0˜30% Ethyl Acetate in petroleum ether) to give the title product 1-{3-chloro-5-[(3R)-3-methylmorpholin-4-yl]-[1,2]thiazolo[4,5-b]pyridin-7-yl}cyclopentane-1-carbonitrile (140 mg, 0.386 mmol, 78.37%). LC-MS(ESI+): m/z (M+H)=362.9, 364.8

### Step 2. 1-(3-(3-methyl-1-(tetrahydro-2H-pyran-2-yl)-1H-pyrazol-5-yl)-5-((R)-3-methylmorpholino)isothiazolo[4,5-b]pyridin-7-yl)cyclopentane-1-carbonitrile

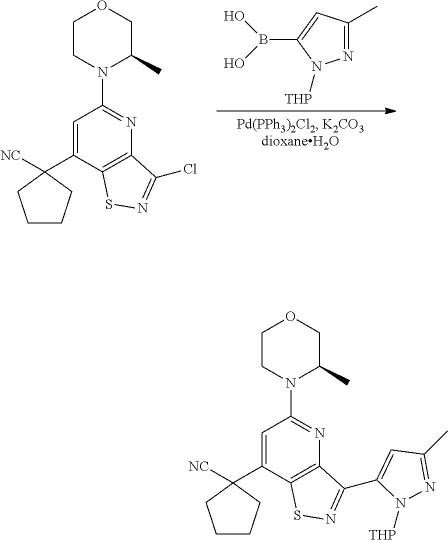

A mixture of 1-{3-chloro-5-[(3R)-3-methylmorpholin-4-yl]-[1,2]thiazolo[4,5-b]pyridin-7-yl}cyclopentane-1-carbonitrile (140 mg, 0.386 mmol), [3-methyl-1-(oxan-2-yl)-1H-pyrazol-5-yl]boronic acid (324.13 mg, 1.543 mmol), Pd(dppf)Cl₂ (56.46 mg, 0.077 mmol) and K₂CO₃ (266.60 mg, 1.929 mmol) in dioxane (10 mL) and water (1 mL) was stirred overnight at 100° C. under nitrogen atmosphere.

The reaction mixture was diluted with water and extracted with ethyl acetate. The combined organic layer was washed with brine, dried over Na₂SO₄, filtered and concentrated in vacuo. The residue was purified on flash column chromatography (Silica, 0˜80% Ethyl Acetate in petroleum ether) to give the title product 1-{3-[3-methyl-1-(oxan-2-yl)-1H-pyrazol-5-yl]-5-[(3R)-3-methylmorpholin-4-yl]-[1,2]thiazolo[4,5-b]pyridin-7-yl}cyclopentane-1-carbonitrile (91 mg, 0.185 mmol, 47.88%). LC-MS(ESI+): m/z (M+H-THP)=408.9

### Step 3. (R)-1-(3-(3-methyl-1H-pyrazol-5-yl)-5-(3-methylmorpholino)isothiazolo[4,5-b]pyridin-7-yl)cyclopentane-1-carbonitrile

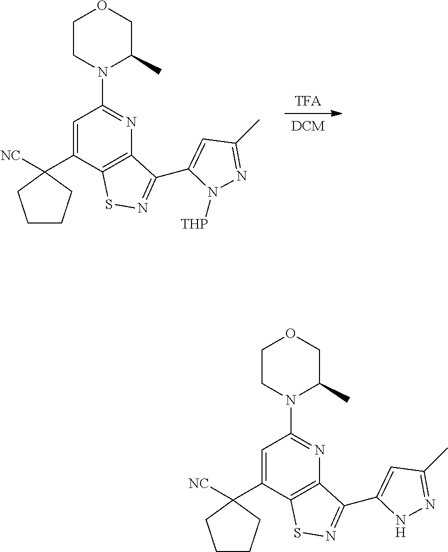

To a solution of 1-{3-[3-methyl-1-(oxan-2-yl)-1H-pyrazol-5-yl]-5-[(3R)-3-methylmorpholin-4-yl]-[1,2]thiazolo[4,5-b]pyridin-7-yl}cyclopentane-1-carbonitrile (91 mg, 0.185 mmol) in DCM (5 mL) was added TFA (5 mL) and the resulting mixture was stirred for 3 hrs at ambient temperature. The mixture was concentrated and basified with saturated ammonium. The mixture was concentrated and the residue was purified on flash column chromatography (Silica, 0˜10% MeOH in DCM) and Prep-HPLC (C18, 10-95%, acetonitrile in water with 0.1% formic acid) to give the title product 1-[3-(3-methyl-1H-pyrazol-5-yl)-5-[(3R)-3-methylmorpholin-4-yl]-[1,2]thiazolo[4,5-b]pyridin-7-yl]cyclopentane-1-carbonitrile (44.2 mg, 0.108 mmol, 58.57%). LC-MS(ESI+): m/z (M+H)=408.9. ¹H NMR (400 MHz, DMSO) δ 13.10 (d, J=123.6 Hz, 1H), 7.15 (d, J=14.6 Hz, 2H), 4.56 (s, 1H), 4.13 (d, J=12.3 Hz, 1H), 4.04 (d, J=9.8 Hz, 1H), 3.82 (d, J=11.2 Hz, 1H), 3.71 (dd, J=11.4, 2.7 Hz, 1H), 3.62-3.51 (m, 1H), 3.30-3.23 (m, 1H), 2.63-2.55 (m, 2H), 2.39-2.27 (m, 5H), 2.01-1.91 (m, 4H), 1.24 (d, J=6.6 Hz, 3H).

### Example 81

### Synthesis of (R)-1-(3-(3-methyl-1H-pyrazol-5-yl)-5-(3-methylmorpholino)isothiazolo[4,5-b]pyridin-7-yl)cyclohexane-1-carbonitrile

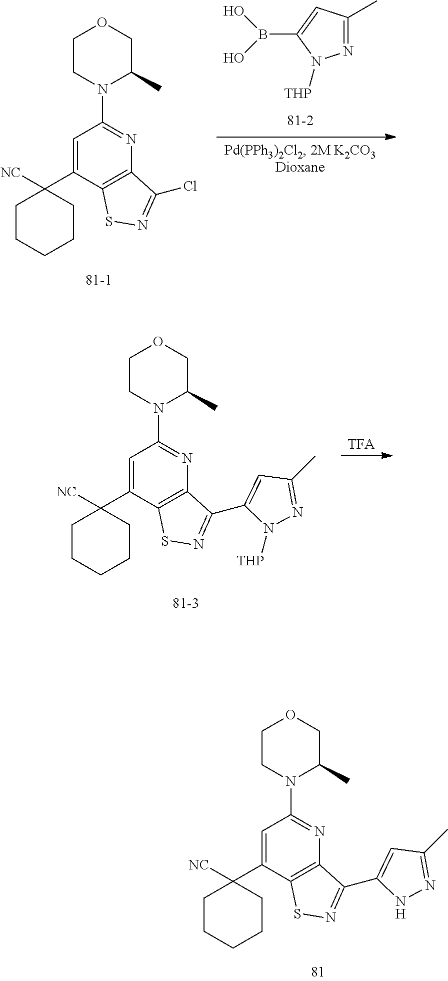

### Step 1. 1-(3-(3-methyl-1-(tetrahydro-2H-pyran-2-yl)-1H-pyrazol-5-yl)-5-((R)-3-methylmorpholino)isothiazolo[4,5-b]pyridin-7-yl)cyclohexane-1-carbonitrile

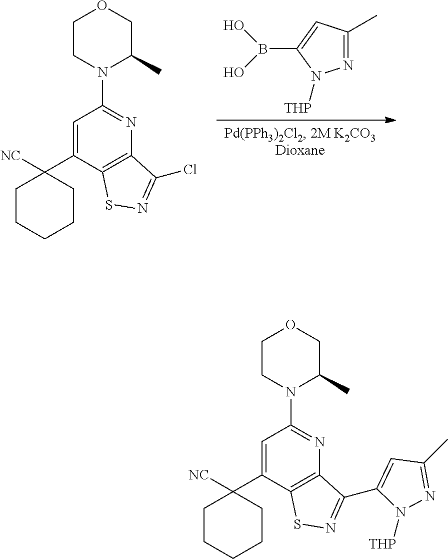

A mixture of 1-{3-chloro-5-[(3R)-3-methylmorpholin-4-yl]-[1,2]thiazolo[4,5-b]pyridin-7-yl}cyclohexane-1-carbonitrile (130 mg, 0.34 mmol), [3-methyl-1-(oxan-2-yl)-1H-pyrazol-5-yl]boronic acid (290 mg, 1.38 mmol), PdCl₂(dppf) (50 mg, 0.06 mmol) and K₂CO₃ (2.0 M in H₂O, 0.70 mL, 1.40 mmol) in dioxane (5 mL) was stirred at 100° C. for 16 h under N₂ atmosphere. LC-MS showed the reaction was complete. The reaction mixture was diluted with H₂O (20 mL), then extracted with EA (50 mL×3). The combined organic layer was washed with brine, dried over anhydrous Na₂SO₄, filtered and concentrated. The residue was purified by column chromatography on silica gel (DCM:MeOH=40:1, V/V) to give the desired product (139 mg, yield: 79%). LC/MS (ESI): m/z 507 [M+H]⁺.

### Step 2. (R)-1-(3-(3-methyl-1H-pyrazol-5-yl)-5-(3-methylmorpholino)isothiazolo[4,5-b]pyridin-7-yl)cyclohexane-1-carbonitrile

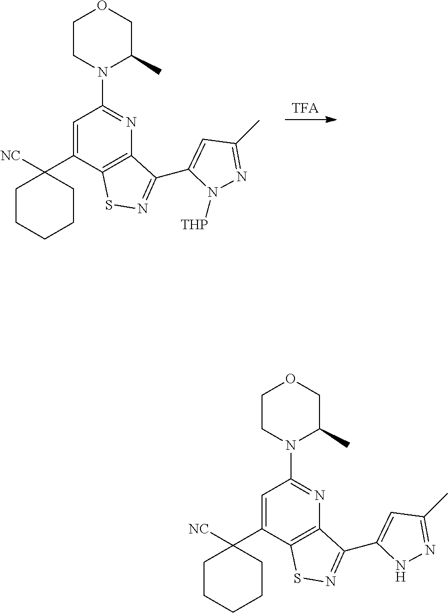

A mixture of 1-{3-[3-methyl-1-(oxan-2-yl)-1H-pyrazol-5-yl]-5-[(3S)-3-methylmorpholin-4-yl]-[1,2]thiazolo[4,5-b]pyridin-7-yl}cyclohexane-1-carbonitrile (139 mg, 0.27 mmol) in TFA (5.0 mL) was stirred at 30° C. for 2 h. LC-MS showed the reaction was complete. The reaction mixture was concentrated under reduced pressure. The residue was purified by prep-HPLC (C18, 10-95%, MeOH in H₂O with 0.1% HCOOH) to give the desired product (20 mg, yield: 17%). LC/MS (ESI): m/z 423 [M+H]⁺. 1H NMR (400 MHz, DMSO-d6) δ 13.10 (d, J=123.2 Hz, 1H), 7.15 (dd, J=26.7, 15.1 Hz, 2H), 4.55 (s, 1H), 4.09 (dd, J=35.7, 11.7 Hz, 2H), 3.83 (d, J=11.2 Hz, 1H), 3.71 (d, J=9.1 Hz, 1H), 3.57 (t, J=10.7 Hz, 1H), 3.27 (s, 1H), 2.38-2.27 (m, 5H), 2.10-2.01 (m, 2H), 1.93 (d, J=14.1 Hz, 2H), 1.74 (dt, J=39.1, 13.3 Hz, 3H), 1.37 (dd, J=17.4, 8.4 Hz, 1H), 1.25 (d, J=6.5 Hz, 3H).

### Example 82

### Synthesis of 2-[3-(3-methyl-1H-pyrazol-5-yl)-5-[(3R)-3-methylmorpholin-4-yl]-[1,2]thiazolo[4,5-b]pyridin-7-yl]-1λ\hat{ }6,2-thiazinane-1,1-dione

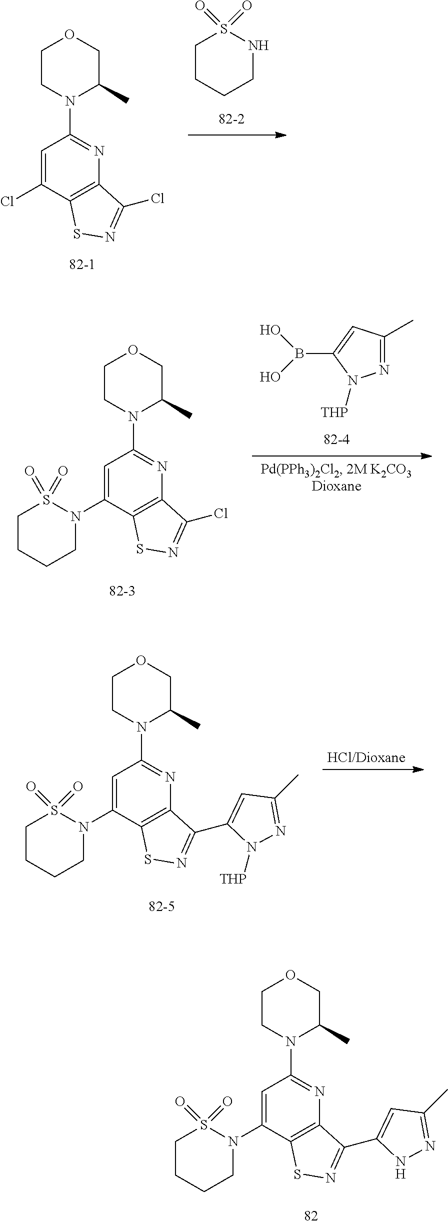

### Step 1. 2-{3-chloro-5-[(3R)-3-methylmorpholin-4-yl]-[1,2]thiazolo[4,5-b]pyridin-7-yl}-1λ\hat{ }6,2-thiazinane-1,1-dione

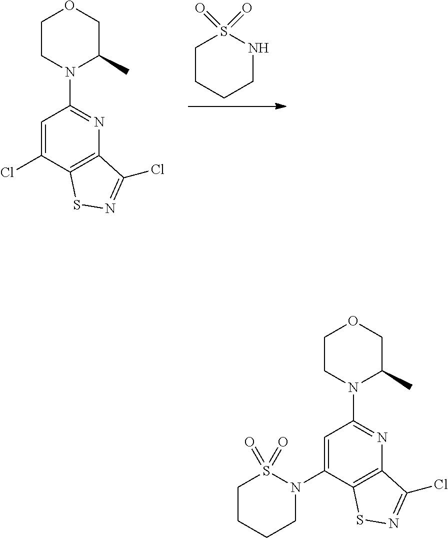 [SMILES: C[C@@H]1COCCN1c1cc(Cl)c2snc(Cl)c2n1 ; O=S1(=O)CCCCN1 ; C[C@@H]1COCCN1c1cc(N2CCCCS2(=O)=O)c2snc(Cl)c2n1]

To a solution of (3R)-4-{3,7-dichloro-[1,2]thiazolo[4,5-b]pyridin-5-yl}-3-methylmorpholine (200 mg, 0.657 mmol) and 1λ\hat{ }6,2-thiazinane-1,1-dione (177.76 mg, 1.315 mmol) in toluene (8 mL) were added Pd(OAc)₂ (14.76 mg, 0.066 mmol), XANT PHOS (76.08 mg, 0.131 mmol) and CS₂CO₃ (428.43 mg, 1.315 mmol), and the reaction was stirred at 100° C. overnight under nitrogen atmosphere. The reaction was diluted with DCM and water. The organic layer was separated, washed with further saturated NaCl solution, and concentrated in vacuo. The residue was purified via Biotage (PE:EA=2:1) to afford the 2-{3-chloro-5-[(3R)-3-methylmorpholin-4-yl]-[1,2]thiazolo[4,5-b]pyridin-7-yl}-1λ\hat{ }6,2-thiazinane-1,1-dione (110 mg, 0.273 mmol, 41.52%). LC/MS (ESI) m/z:403 (M+H)⁺.

### Step 2. 2-{3-[3-methyl-1-(oxan-2-yl)-1H-pyrazol-5-yl]-5-[(3R)-3-methylmorpholin-4-yl]-[1,2]thiazolo[4,5-b]pyridin-7-yl}-1λ\hat{ }6,2-thiazinane-1,1-dione

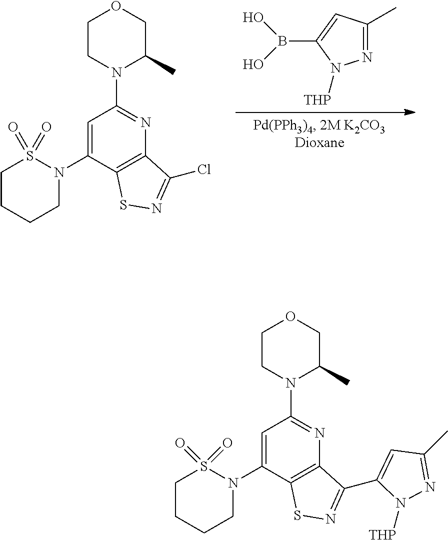

To a solution of 2-{3-chloro-5-[(3R)-3-methylmorpholin-4-yl]-[1,2]thiazolo[4,5-b]pyridin-7-yl}-1λ\hat{ }6,2-thiazinane-1,1-dione (100 mg, 0.248 mmol) in dioxane (10 mL) was added [3-methyl-1-(oxan-2-yl)-1H-pyrazol-5-yl]boronic acid (156.38 mg, 0.745 mmol), Pd(PPh₃)₄ (28.68 mg, 0.025 mmol), K₂CO₃ (68.60 mg, 0.496 mmol), and the reaction was stirred at 100° C. overnight under nitrogen atmosphere. The reaction was diluted with EA and water. The organic layer was separated, washed with further saturated NaCl solution, and concentrated in vacuo to give the title product 2-{3-[3-methyl-1-(oxan-2-yl)-1H-pyrazol-5-yl]-5-[(3R)-3-methylmorpholin-4-yl]-[1,2]thiazolo[4,5-b]pyridin-7-yl}-1λ\hat{ }6,2-thiazinane-1,1-dione (50 mg, 0.094 mmol, 37.82%). LC/MS (ESI) m/z:533 (M+H)⁺.

### Step 3. 2-[3-(3-methyl-1H-pyrazol-5-yl)-5-[(3R)-3-methylmorpholin-4-yl]-[1,2]thiazolo[4,5-b]pyridin-7-yl]-1λ\hat{ }6,2-thiazinane-1,1-dione

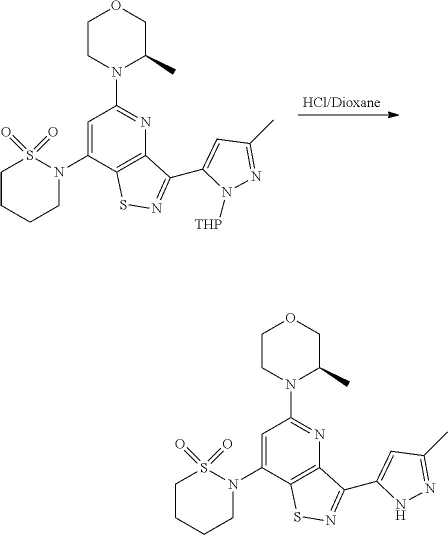

To a solution of 2-{3-[3-methyl-1-(oxan-2-yl)-1H-pyrazol-5-yl]-5-[(3R)-3-methylmorpholin-4-yl]-[1,2]thiazolo[4,5-b]pyridin-7-yl}-1λ\hat{ }6,2-thiazinane-1,1-dione (50 mg, 0.094 mmol) in DCM (5 mL) were added HCl/Dioxane (5 mL), and the reaction was stirred at room temperature for 1 hour. The reaction mixture was extracted with Ethyl Acetate, washed with H₂O and brine, dried over Na₂SO₄, filtered and concentrated in vacuo to give the title product 2-[3-(3-methyl-1H-pyrazol-5-yl)-5-[(3R)-3-methylmorpholin-4-yl]-[1,2]thiazolo[4,5-b]pyridin-7-yl]-1λ\hat{ }6,2-thiazinane-1,1-dione (11 mg, 0.025 mmol, 26.13%). LC/MS (ESI) m/z:449 (M+H)⁺. ¹H NMR (400 MHz, DMSO-d6) δ 13.07 (d, J=112.8 Hz, 1H), 7.09 (d, J=9.6 Hz, 2H), 4.51 (s, 1H), 4.19-3.96 (m, 2H), 3.91-3.75 (m, 3H), 3.72 (dd, J=11.5, 2.8 Hz, 1H), 3.57 (td, J=11.8, 2.9 Hz, 1H), 3.53-3.46 (m, 2H), 3.30-3.15 (m, 1H), 2.31 (s, 3H), 2.22 (s, 2H), 1.88 (s, 2H), 1.24 (d, J=6.6 Hz, 3H).

### Example 83

### Synthesis of (R)-4-(5-(3-methylmorpholino)-3-(1H-pyrazol-5-yl)isothiazolo[4,5-b]pyridin-7-yl)tetrahydro-2H-pyran-4-carbonitrile

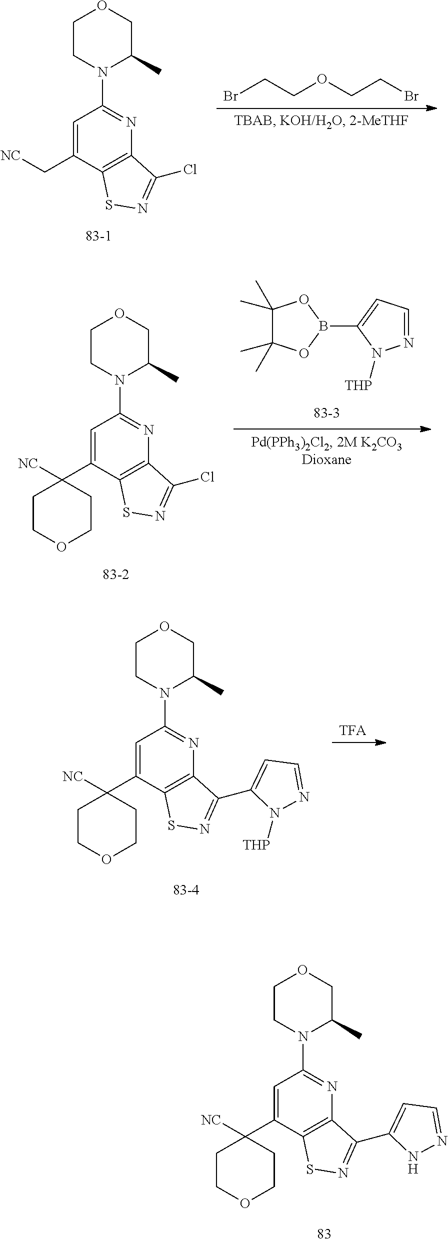

### Step 1. (R)-4-(3-chloro-5-(3-methylmorpholino)isothiazolo[4,5-b]pyridin-7-yl)tetrahydro-2H-pyran-4-carbonitrile

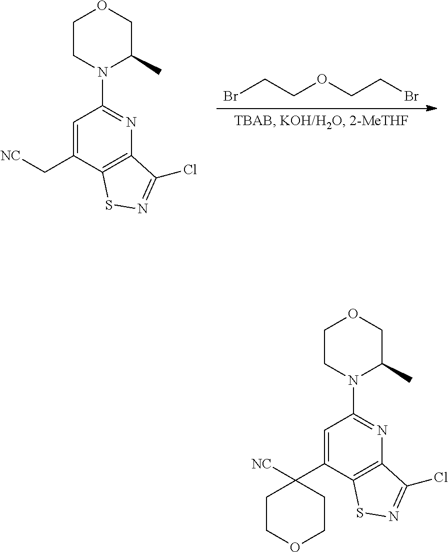

A mixture of 2-{3-chloro-5-[(3R)-3-methylmorpholin-4-yl]-[1,2]thiazolo[4,5-b]pyridin-7-yl}acetonitrile (260 mg, 0.84 mmol), 1-bromo-2-(2-bromoethoxy)ethane (783 mg, 3.37 mmol), KOH (10.0 M in H₂O, 1.6 mL, 16.0 mmol) and TBAB (54 mg, 0.16 mmol) in 2-MTHF (16 mL) was stirred at 80° C. for 2 h. LC-MS showed the reaction was complete. The reaction mixture was diluted with EA (60 mL), then washed with water and brine, dried over anhydrous Na₂SO₄, filtered and concentrated. The residue was purified by column chromatography on silica gel (PE:EA=1:1, V/V) to afford the desired product (134 mg, yield: 42%). LC/MS (ESI): m/z 379 [M+H]⁺.

### Step 2. 4-(5-((R)-3-methylmorpholino)-3-(1-(tetrahydro-2H-pyran-2-yl)-1H-pyrazol-5-yl)isothiazolo[4,5-b]pyridin-7-yl)tetrahydro-2H-pyran-4-carbonitrile

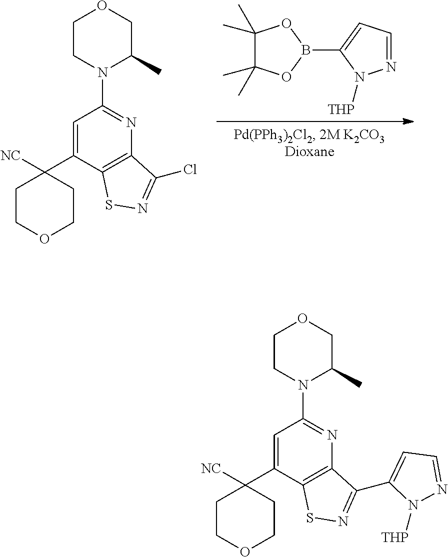

A mixture of 4-{3-chloro-5-[(3R)-3-methylmorpholin-4-yl]-[1,2]thiazolo[4,5-b]pyridin-7-yl}oxane-4-carbonitrile (60 mg, 0.15 mmol), 1-(oxan-2-yl)-5-(tetramethyl-1,3,2-dioxaborolan-2-yl)-1H-pyrazole (110 mg, 0.39 mmol), PdCl₂(dppf) (23 mg, 0.03 mmol) and K₂CO₃ (2.0 M in H₂O, 0.23 mL, 0.47 mmol) in dioxane (2 mL) was stirred at 100° C. for 16 h under N₂ atmosphere. LC-MS showed the reaction was complete. The reaction mixture was diluted with H₂O (20 mL), then extracted with EA (50 mL×3). The combined organic layer was washed with brine, dried over anhydrous Na₂SO₄, filtered and concentrated. The residue was purified by column chromatography on silica gel (DCM:MeOH=40:1, V/V) to give the desired product (40 mg, yield: 51%). LC/MS (ESI): m/z 495 [M+H]⁺.

### Step 3. (R)-4-(5-(3-methylmorpholino)-3-(1H-pyrazol-5-yl)isothiazolo[4,5-b]pyridin-7-yl)tetrahydro-2H-pyran-4-carbonitrile

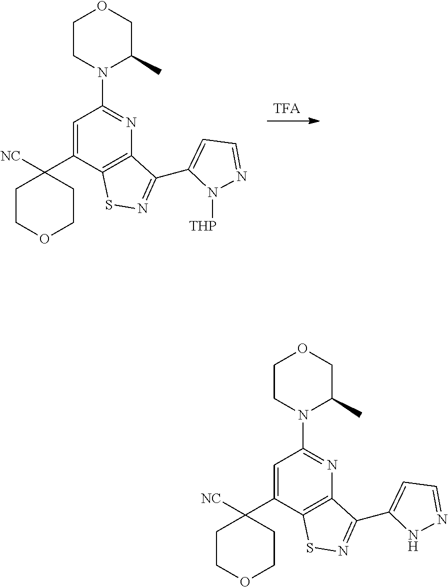

A mixture of 4-{5-[(3S)-3-methylmorpholin-4-yl]-3-[1-(oxan-2-yl)-1H-pyrazol-5-yl]-[1,2]thiazolo[4,5-b]pyridin-7-yl}oxane-4-carbonitrile (47 mg, 0.09 mmol) in TFA (3.0 mL) was stirred at 30° C. for 1 h. LC-MS showed the reaction was complete. The reaction mixture was concentrated under reduced pressure. The residue was purified by Prep-HPLC (C18, 10-95%, MeOH in H₂O with 0.1% HCOOH) to give the desired product (10 mg, yield: 25%). LC/MS (ESI): m/z 411 [M+H]⁺. 1H NMR (400 MHz, DMSO) δ 13.51 (d, J=174.9 Hz, 1H), 7.80 (d, J=87.4 Hz, 1H), 7.40 (d, J=1.6 Hz, 1H), 7.21 (s, 1H), 4.59 (s, 1H), 4.20-4.00 (m, 4H), 3.87-3.68 (m, 4H), 3.56 (dd, J=11.6, 9.0 Hz, 1H), 3.27 (d, J=13.0 Hz, 1H), 2.38-2.27 (m, 4H), 1.25 (d, J=6.7 Hz, 3H).

### Example 84

### Synthesis of (R)-1-(3-(3-methyl-1H-pyrazol-5-yl)-5-(3-methylmorpholino)isothiazolo[4,5-b]pyridin-7-yl)cyclopentane-1-carbonitrile

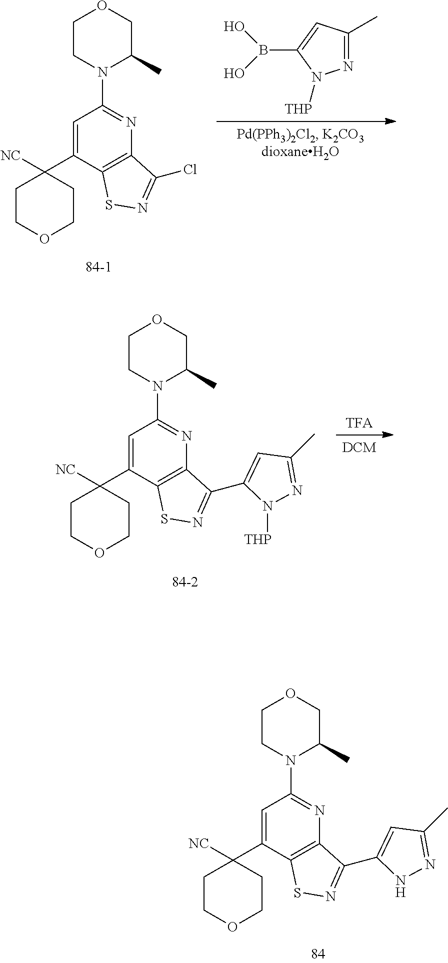

### Step 1. 4-(3-(3-methyl-1-(tetrahydro-2H-pyran-2-yl)-1H-pyrazol-5-yl)-5-((R)-3-methylmorpholino)isothiazolo[4,5-b]pyridin-7-yl)tetrahydro-2H-pyran-4-carbonitrile

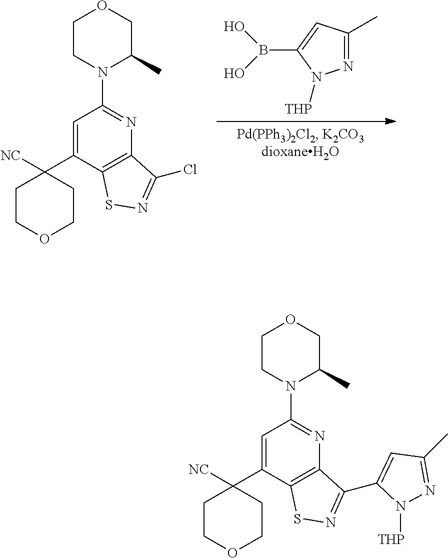

A mixture of 4-{3-chloro-5-[(3R)-3-methylmorpholin-4-yl]-thiazolo[4,5-b]pyridin-7-yl}oxane-4-carbonitrile (60 mg, 0.158 mmol), [3-methyl-1-(oxan-2-yl)-1H-pyrazol-5-yl]boronic acid (133.05 mg, 0.633 mmol), Pd(dppf)Cl₂ (23.17 mg, 0.032 mmol) and K₂CO₃ (552.84 mg, 4 mmol) in dioxane (10 mL) and water (2 mL) was stirred overnight at 100° C. under nitrogen atmosphere. The reaction mixture was diluted with water and extracted with ethyl acetate. The combined organic layer was washed with brine, dried over Na₂SO₄, filtered and concentrated in vacuo. The residue was purified on flash column chromatography (Silica, 0˜100% Ethyl Acetate in petroleum ether) to give the title product 4-{3-[3-methyl-1-(oxan-2-yl)-1H-pyrazol-5-yl]-5-[(3R)-3-methylmorpholin-4-yl]-[1,2]thiazolo[4,5-b]pyridin-7-yl}oxane-4-carbonitrile (47 mg, 0.092 mmol, 58.35%). LC-MS(ESI+): m/z (M+H)=508.9

### Step 2. (R)-4-(3-(3-methyl-1H-pyrazol-5-yl)-5-(3-methylmorpholino)isothiazolo[4,5-b]pyridin-7-yl)tetrahydro-2H-pyran-4-carbonitrile

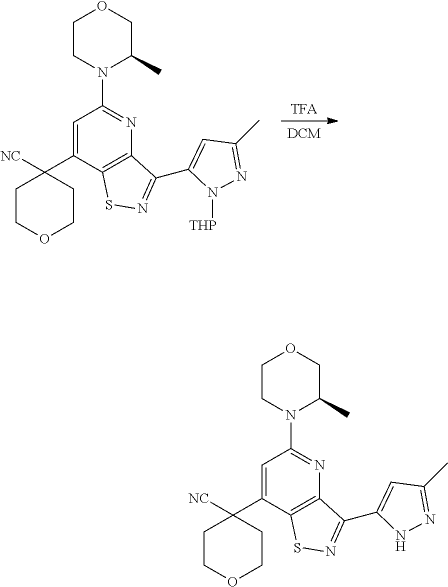

To a solution of 4-{3-[3-methyl-1-(oxan-2-yl)-1H-pyrazol-5-yl]-5-[(3R)-3-methylmorpholin-4-yl]-[1,2]thiazolo[4,5-b]pyridin-7-yl}oxane-4-carbonitrile (47 mg, 0.092 mmol) in DCM (3 mL) was added TFA (3 mL) and the resulting mixture was stirred for 3 hrs at ambient temperature. The mixture was concentrated and basified with saturated ammonium. The mixture was concentrated and the residue was purified on flash column chromatography (Silica, 0˜10% MeOH in DCM) and Prep-HPLC (C18, 10-95%, acetonitrile in water with 0.1% formic acid) to give the title product 4-[3-(3-methyl-1H-pyrazol-5-yl)-5-[(3R)-3-methylmorpholin-4-yl]-[1,2]thiazolo[4,5-b]pyridin-7-yl]oxane-4-carbonitrile (16.2 mg, 0.038 mmol, 41.29%). LC-MS(ESI+): m/z (M+H)=424.8. ¹H NMR (400 MHz, DMSO) δ 13.11 (d, J=123.3 Hz, 1H), 7.29-7.04 (m, 2H), 4.58 (s, 1H), 4.19-4.01 (m, 4H), 3.86-3.68 (m, 4H), 3.62-3.52 (m, 1H), 3.31-3.23 (m, 1H), 2.39-2.25 (m, 7H), 1.25 (d, J=6.6 Hz, 3H).

### Example 85

### Synthesis of (R)-4-(7-(cyclopropylsulfonyl)-3-(1H-pyrazol-5-yl)isothiazolo[4,5-b]pyridin-5-yl)-3-methylmorpholine

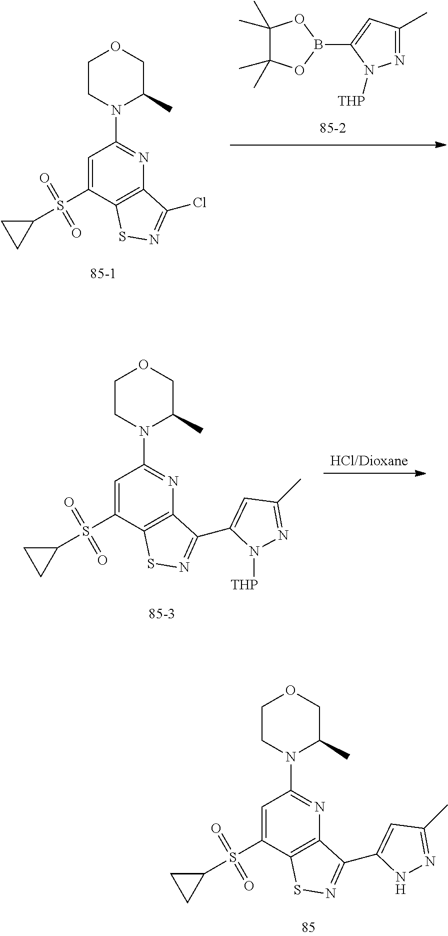

### Step 1. (R)-4-(7-(cyclopropylsulfonyl)-3-(3-methyl-1H-pyrazol-5-yl)isothiazolo[4,5-b]pyridin-5-yl)-3-methylmorpholine

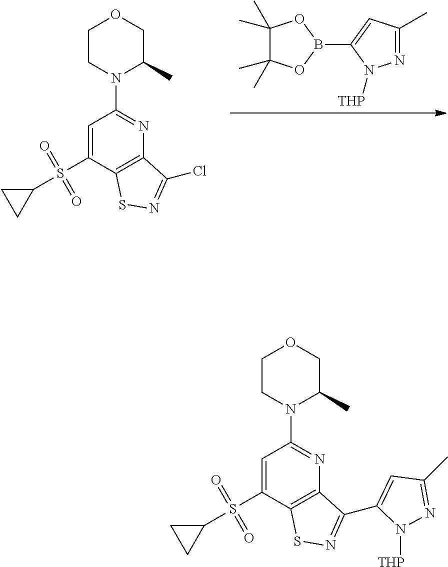

To a solution of (R)-4-(3-chloro-7-(cyclopropylsulfonyl)isothiazolo[4,5-b]pyridin-5-yl)-3-methylmorpholine (70 mg, 0.187 mmol) in dioxane (3.0 mL) was added 3-methyl-1-(tetrahydro-2H-pyran-2-yl)-5-(4,4,5,5-tetramethyl-1,3,2-dioxaborolan-2-yl)-1H-pyrazole (117.97 mg, 0.562 mmol), K₂CO₃ (0.468 mL, 0.936 mmol) and Pd(PPh₃)₄ (21.63 mg, 0.019 mmol), and the reaction was stirred at 100° C. overnight under nitrogen atmosphere. LC-MS showed the reaction was complete. The reaction was diluted with EA (10 mL) and water (10 mL). The organic layer was separated, washed with further saturated NaCl solution, dried over anhydrous Na₂SO₄, filtered and concentrated in vacuo. The residue was purified by prep-TLC (DCM:MeOH=30:1, V/V) to afford the desired product (80 mg, 0.159 mmol, 84.84%). LC/MS (ESI) m/z: 504 (M+H)⁺.

### Step 2. (R)-4-(7-(cyclopropylsulfonyl)-3-(3-methyl-1H-pyrazol-5-yl)isothiazolo[4,5-b]pyridin-5-yl)-3-methylmorpholine

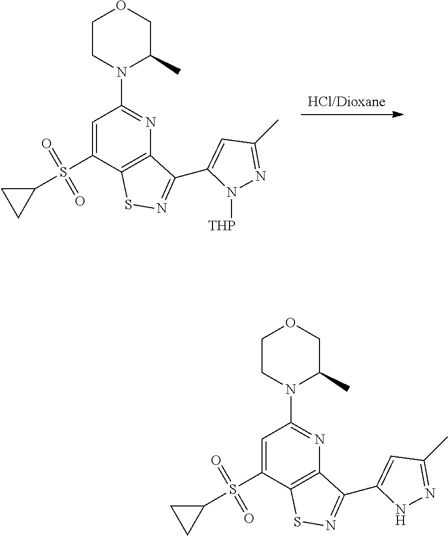

A solution of (3R)-4-(7-(cyclopropylsulfonyl)-3-(3-methyl-1-(tetrahydro-2H-pyran-2-yl)-1H-pyrazol-5-yl)isothiazolo[4,5-b]pyridin-5-yl)-3-methylmorpholine (80 mg, 0.159 mmol) in HCl solution (4M in dioxane, 2 mL) was stirred at room temperature for 1 hr. LC-MS showed the reaction was complete. The reaction mixture was concentrated in vacuo. The residue was purified by prep-HPLC (C₁₈, 10-95%, MeOH in H₂O with 0.1% TFA) to afford the desired product (35 mg, 0.083 mmol, 52.52%). LC/MS (ESI) m/z: 420 (M+H)⁺. ¹H NMR (400 MHz, DMSO-d6) δ 7.67 (s, 1H), 7.10 (s, 1H), 4.60 (d, J=6.4 Hz, 1H), 4.19 (d, J=11.9 Hz, 1H), 4.09-4.03 (m, 1H), 3.84 (d, J=11.4 Hz, 1H), 3.73 (dd, J=11.4, 2.8 Hz, 1H), 3.59 (d, J=2.8 Hz, 1H), 3.32 (dd, J=12.6, 9.0 Hz, 1H), 3.22-3.17 (m, 1H), 2.32 (s, 3H), 1.28 (m, 5H), 1.16 (dd, J=7.8, 2.4 Hz, 2H).

### Example 86

### Synthesis of (R)-1-(3-(3-methyl-1H-pyrazol-5-yl)-5-(3-methylmorpholino)isothiazolo[4,5-b]pyridin-7-yl)cyclohexan-1-ol

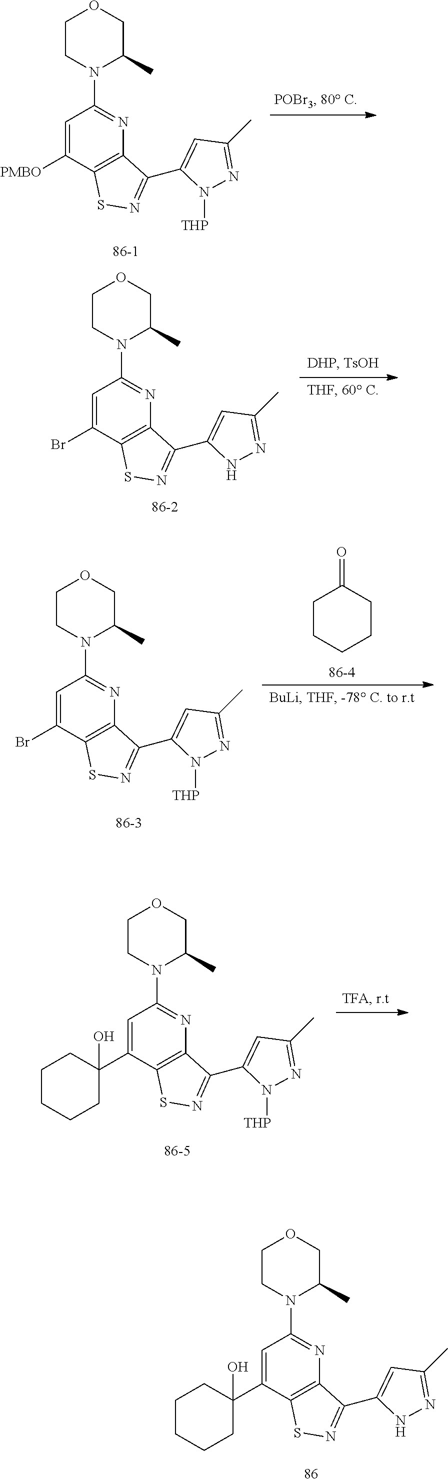

### Step 1. (R)-4-(7-bromo-3-(3-methyl-1H-pyrazol-5-yl)isothiazolo[4,5-b]pyridin-5-yl)-3-methylmorpholine

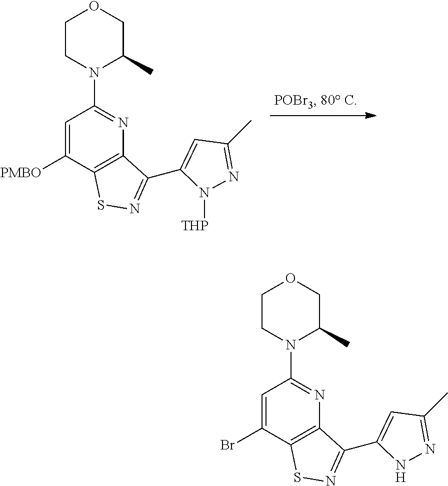

A mixture of (3R)-4-{7-[(4-methoxyphenyl)methoxy]-3-[3-methyl-1-(oxan-2-yl)-1H-pyrazol-5-yl]-[1,2]thiazolo[4,5-b]pyridin-5-yl}-3-methylmorpholine (200 mg, 0.37 mmol) and POBr₃ (200 mg, 0.37 mmol) was stirred at 80° C. under N₂ atmosphere for 3 h. The reaction mixture was diluted with DCM and washed with H₂O. The organic layer was dried over Na₂SO₄, filtered and concentrated in vacuum.

The residue was purified on flash column eluting with DCM:MeOH=20:1 to give the desired product (50 mg, yield: 33%). LC/MS (ESI): m/z 394 [M+H]⁺.

### Step 2. (3R)-4-(7-bromo-3-(3-methyl-1-(tetrahydro-2H-pyran-2-yl)-1H-pyrazol-5-yl)isothiazolo[4,5-b]pyridin-5-yl)-3-methylmorpholine

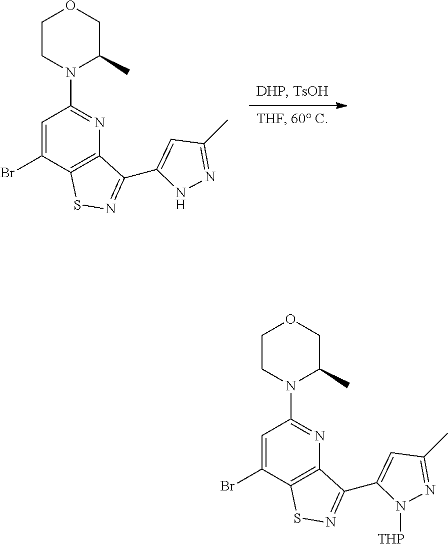

A mixture of (3R)-4-[7-bromo-3-(3-methyl-1H-pyrazol-5-yl)-[1,2]thiazolo[4,5-b]pyridin-5-yl]-3-methylmorpholine (100 mg, 0.25 mmol), DHP (95 mg, 1.14 mmol) and TsOH (8 mg, 0.05 mmol) in THF (5 mL) was stirred at 65° C. for 16 h. LCMS showed the reaction was completed. The reaction mixture was diluted with EA and washed with H₂O. The organic layer was dried over anhydrous Na₂SO₄, filtered and concentrated. The residue was purified by column chromatography on silica gel (PE:EA=2:1, V/V) to afford the desired product (38 mg, yield: 31%). LC/MS(ESI): m/z 478 [M+H]⁺.

### Step 3. 1-(3-(3-methyl-1-(tetrahydro-2H-pyran-2-yl)-1H-pyrazol-5-yl)-5-((R)-3-methylmorpholino)isothiazolo[4,5-b]pyridin-7-yl)cyclohexan-1-ol

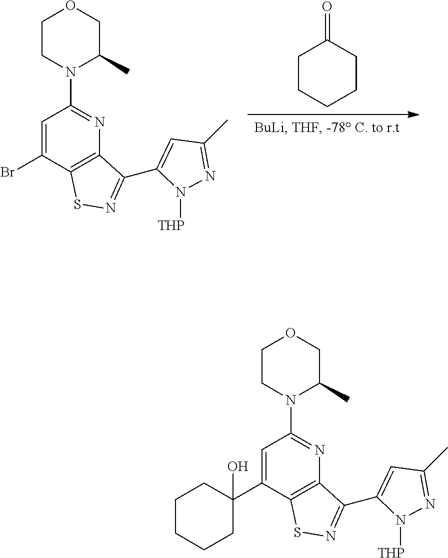

To a solution of (3R)-4-{7-bromo-3-[3-methyl-1-(oxan-2-yl)-1H-pyrazol-5-yl]-[1,2]thiazolo[4,5-b]pyridin-5-yl}-3-methylmorpholine (38 mg, 0.08 mmol) and cyclohexanone (39 mg, 0.39 mmol) in anhydrous THF (3 mL) was added n-BuLi (2.5 M in hexane, 0.12 mL, 0.32 mmol) slowly. The resulting mixture was stirred at −78° C. under N₂ atmosphere for 1 h. LCMS showed the reaction was completed. The reaction mixture was quenched with NaHCO₃ aqueous solution and extracted with EA. The combined organic layer was dried over Na₂SO₄, filtered and concentrated in vacuum. The residue was purified by column chromatography on silica gel (PE:EA=1:1, V/V) to afford the desired product (17 mg, yield: 43%). LC/MS (ESI): m/z 498 [M+H]⁺.

### Step 4. (R)-1-(3-(3-methyl-1H-pyrazol-5-yl)-5-(3-methylmorpholino)isothiazolo[4,5-b]pyridin-7-yl)cyclohexan-1-ol

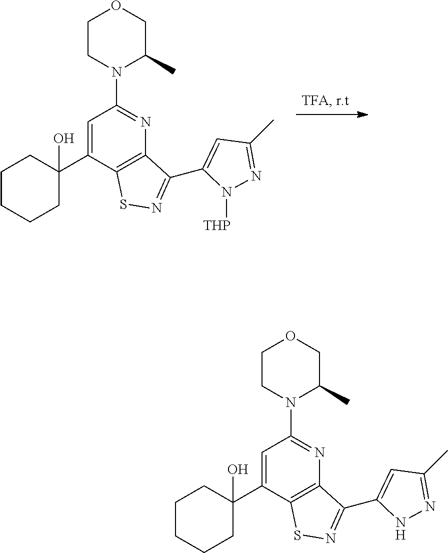

A mixture of 1-{3-[3-methyl-1-(oxan-2-yl)-1H-pyrazol-5-yl]-5-[(3R)-3-methylmorpholin-4-yl]-[1,2]thiazolo[4,5-b]pyridin-7-yl}cyclohexan-1-ol (22 mg, 0.04 mmol) in DCM/TFA (V/V, 2 mL/1 mL) was stirred at room temperature for 16 h.

After concentration, the residue was purified by prep-HPLC (C18, 10-95%, MeOH in H₂O with 0.1% HCOOH) to give the desired product (3 mg, yield: 16%). LC/MS (ESI): m/z 414 [M+H]⁺. ¹H NMR (400 MHz, DMSO-d6) δ 13.01 (s, 1H), 7.09 (s, 1H), 7.05 (s, 1H), 5.83 (s, 1H), 4.55 (d, J=5.8 Hz, 1H), 4.10 (d, J=12.2 Hz, 1H), 4.03 (d, J=8.7 Hz, 1H), 3.81 (d, J=11.2 Hz, 1H), 3.72 (d, J=8.8 Hz, 1H), 3.57 (t, J=10.6 Hz, 1H), 3.20 (d, J=12.7 Hz, 1H), 2.29 (s, 3H), 1.87-1.71 (m, 6H), 1.58 (s, 2H), 1.36 (d, J=12.3 Hz, 1H), 1.25-1.20 (m, 4H).

### Example 87

### Synthesis of (R)-1-(3-(3-methyl-1H-pyrazol-5-yl)-5-(3-methylmorpholino)isothiazolo[4,5-b]pyridin-7-yl)cyclopentane-1-carbonitrile

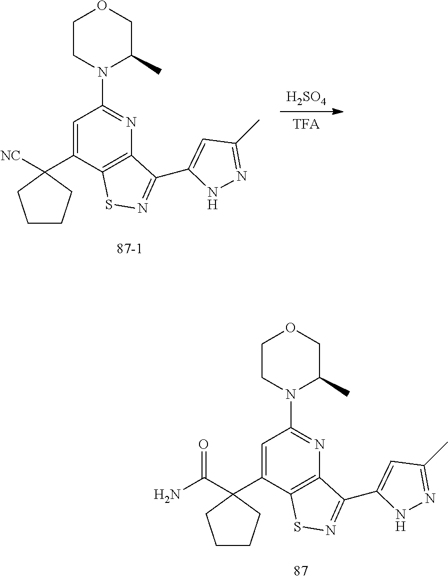 [SMILES: Cc1cc(-c2nsc3c(C4(C#N)CCCC4)cc(N4CCOC[C@H]4C)nc23)[nH]n1 ; "H2SO4" ; Cc1cc(-c2nsc3c(C4(C(N)=O)CCCC4)cc(N4CCOC[C@H]4C)nc23)[nH]n1 ; "TFA"]

### Step 1. (R)-1-(3-chloro-5-(3-methylmorpholino)isothiazolo[4,5-b]pyridin-7-yl)cyclopentane-1-carbonitrile

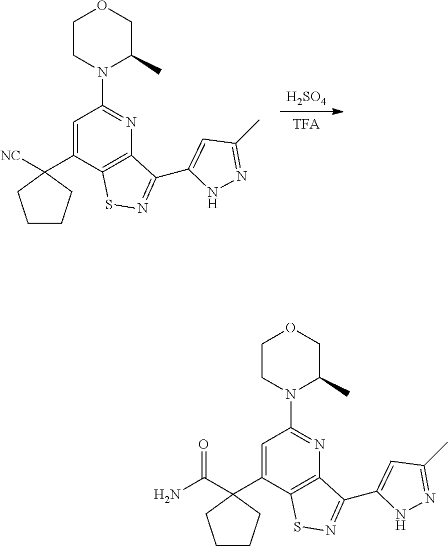 [SMILES: Cc1cc(-c2nsc3c(C4(C#N)CCCC4)cc(N4CCOC[C@H]4C)nc23)[nH]n1 ; "H2SO4" ; Cc1cc(-c2nsc3c(C4(C(N)=O)CCCC4)cc(N4CCOC[C@H]4C)nc23)[nH]n1 ; "TFA"]

To a solution of (R)-1-(3-(3-methyl-1H-pyrazol-5-yl)-5-(3-methylmorpholino)isothiazolo[4,5-b]pyridin-7-yl)cyclopentane-1-carbonitrile (13 mg, 0.0318 mmol) in TFA (3.5 mL) was added concentrated H₂SO₄ (0.5 mL) and the resulting mixture was stirred at 100° C. for 2 hrs under nitrogen atmosphere. The mixture was concentrated and basified with saturated ammonium. The mixture was concentrated and the residue was purified on flash column chromatography (Silica, 0˜10% MeOH in DCM) and Prep-HPLC (C18, 10-95%, acetonitrile in water with 0.1% formic acid) to give the title product (R)-1-(3-(3-methyl-1H-pyrazol-5-yl)-5-(3-methylmorpholino)isothiazolo[4,5-b]pyridin-7-yl)cyclopentane-1-carboxamide (8.8 mg, 0.0206 mmol, 64.83%). LC-MS(ESI+): m/z (M+H)=426.9. 1H NMR (400 MHz, DMSO) δ 7.24-6.97 (m, 4H), 4.52 (d, J=4.8 Hz, 1H), 4.15-4.01 (m, 2H), 3.83 (d, J=11.3 Hz, 1H), 3.75-3.69 (m, 1H), 3.60-3.54 (m, 1H), 3.27-3.23 (m, 1H), 2.67-2.58 (m, 2H), 2.30 (s, 3H), 2.04-1.91 (m, 2H), 1.74-1.63 (m, 4H), 1.26 (d, J=6.7 Hz, 3H).

### Example 88

### Synthesis of (R)-1-(3-(3-methyl-1H-pyrazol-5-yl)-5-(3-methylmorpholino)isothiazolo[4,5-b]pyridin-7-yl)cyclohexane-1-carboxamide

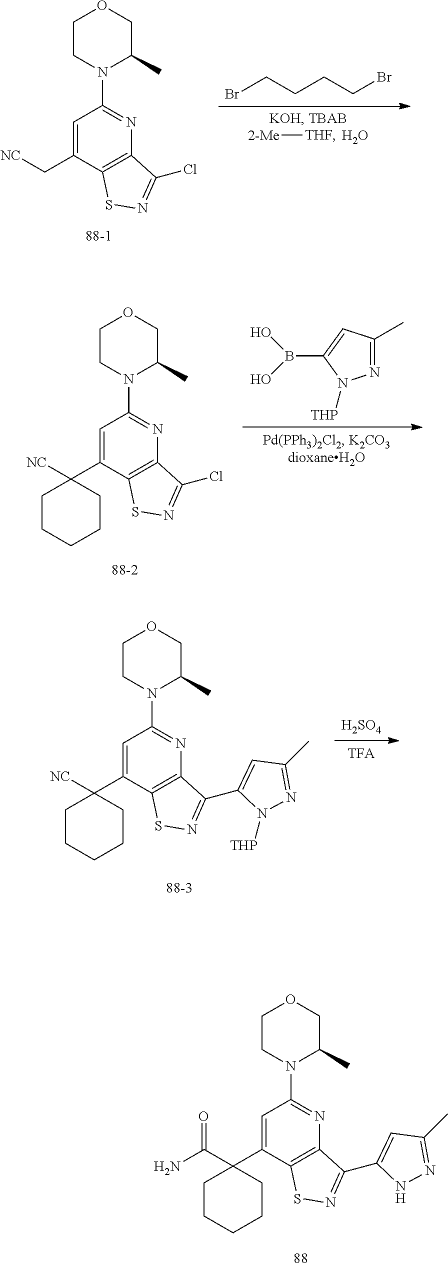

### Step 1. 1-{3-chloro-5-[(3R)-3-methylmorpholin-4-yl]-[1,2]thiazolo[4,5-b]pyridin-7-yl}cyclohexane-1-carbonitrile

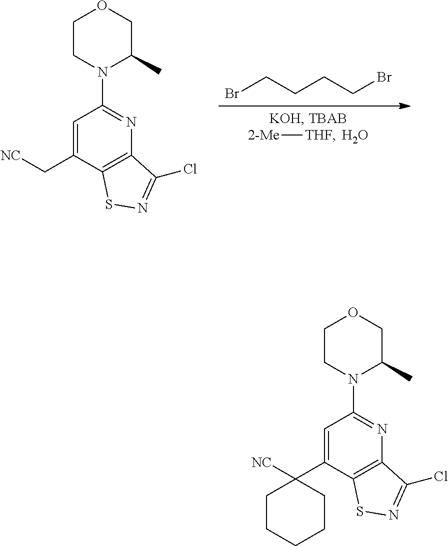

A mixture of 2-{3-chloro-5-[(3R)-3-methylmorpholin-4-yl]-thiazolo[4,5-b]pyridin-7-yl}acetonitrile (174 mg, 0.563 mmol), 1,5-dibromopentane (0.308 mL, 2.254 mmol), KOH (632.35 mg, 11.270 mmol) and TBAB (0.035 mL, 0.113 mmol) in 2-Methyltetrahydrofuran (10 mL) and water (1 mL) was stirred at 80° C. for 4 hrs under nitrogen atmosphere. The reaction mixture was diluted with water and extracted with ethyl acetate. The combined organic layer was washed with brine, dried over Na₂SO₄, filtered and concentrated in vacuo. The residue was purified on flash column chromatography (Silica, 0˜30% Ethyl Acetate in petroleum ether) to give the title product 1-{3-chloro-5-[(3R)-3-methylmorpholin-4-yl]-[1,2]thiazolo[4,5-b]pyridin-7-yl}cyclohexane-1-carbonitrile (169 mg, 0.448 mmol, 79.57%). LC-MS(ESI+): m/z (M+H)=376.9, 378.8 Step 2. 1-{3-[3-methyl-1-(oxan-2-yl)-1H-pyrazol-5-yl]-5-[(3R)-3-methylmorpholin-4-yl]-[1,2]thiazolo[4,5-b]pyridin-7-yl}cyclohexane-1-carbonitrile

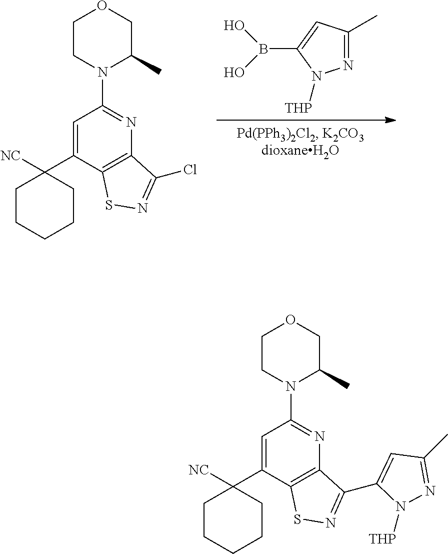

A mixture of 1-{3-chloro-5-[(3R)-3-methylmorpholin-4-yl]-[1,2]thiazolo[4,5-b]pyridin-7-yl}cyclohexane-1-carbonitrile (84 mg, 0.223 mmol), [3-methyl-1-(oxan-2-yl)-1H-pyrazol-5-yl]boronic acid (187.24 mg, 0.891 mmol), Pd(dppf)Cl₂ (32.61 mg, 0.045 mmol) and K₂CO₃ (110.57 mg, 0.8 mmol) in dioxane (2 mL) and water (0.4 mL) was stirred overnight at 100° C. under nitrogen atmosphere.

The reaction mixture was diluted with water and extracted with ethyl acetate. The combined organic layer was washed with brine, dried over Na₂SO₄, filtered and concentrated in vacuo. The residue was purified on flash column chromatography (Silica, 0˜60% Ethyl Acetate in petroleum ether) to give the title product 1-{3-[3-methyl-1-(oxan-2-yl)-1H-pyrazol-5-yl]-5-[(3R)-3-methylmorpholin-4-yl]-[1,2]thiazolo[4,5-b]pyridin-7-yl}cyclohexane-1-carbonitrile (79 mg, 0.156 mmol, 69.96%). LC-MS(ESI+): m/z (M+H)=506.9

### Step 3. 1-[3-(3-methyl-1H-pyrazol-5-yl)-5-[(3R)-3-methylmorpholin-4-yl]-[1,2]thiazolo[4,5-b]pyridin-7-yl]cyclohexane-1-carboxamide

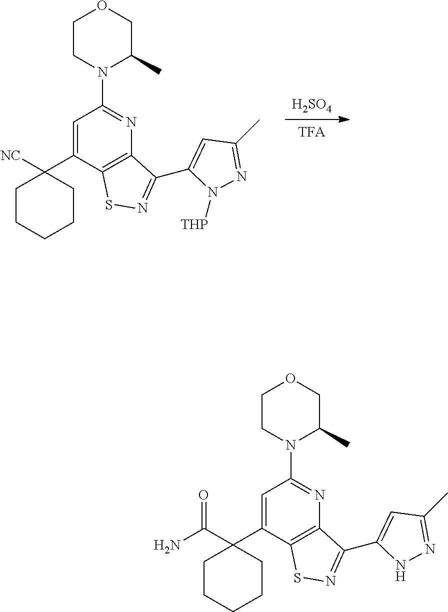

To a solution of 1-{3-[3-methyl-1-(oxan-2-yl)-1H-pyrazol-5-yl]-5-[(3R)-3-methylmorpholin-4-yl]-[1,2]thiazolo[4,5-b]pyridin-7-yl}cyclohexane-1-carbonitrile (79 mg, 0.156 mmol) in TFA (3.5 mL) was added H₂SO₄ (0.5 mL) and the resulting mixture was stirred for 2 hrs at 100° C. under nitrogen atmosphere. The mixture was concentrated and basified with saturated ammonium. The mixture was extracted with ethyl acetate and the organic layer was washed with brine, dried over Na₂SO₄, filtered and concentrated. The residue was purified on Prep-HPLC (C18, 10-95%, acetonitrile in water with 0.1% formic acid) to give the title product 1-[3-(3-methyl-1H-pyrazol-5-yl)-5-[(3R)-3-methylmorpholin-4-yl]-[1,2]thiazolo[4,5-b]pyridin-7-yl]cyclohexane-1-carboxamide (16.4 mg, 0.037 mmol, 23.87%). LC-MS(ESI+): m/z (M+H)=440.9. ¹H NMR (400 MHz, DMSO) δ 12.80 (br, 1H), 7.17 (d, J=18.1 Hz, 2H), 7.08 (d, J=8.5 Hz, 2H), 4.50 (d, J=5.8 Hz, 1H), 4.06 (dd, J=19.6, 8.3 Hz, 2H), 3.83 (d, J=11.3 Hz, 1H), 3.72 (dd, J=11.4, 2.9 Hz, 1H), 3.62-3.52 (m, 1H), 3.29-3.22 (m, 1H), 2.57-2.52 (m, 2H), 2.30 (s, 3H), 1.84-1.75 (m, 2H), 1.65-1.55 (m, 5H), 1.35-1.28 (m, 1H), 1.26 (d, J=6.7 Hz, 3H).

### Example 89

### Synthesis of 1-{5-[(3R)-3-methylmorpholin-4-yl]-3-(1H-pyrazol-5-yl)-[1,2]thiazolo[4,5-b]pyridin-7-yl}cyclohexane-1-carboxamide

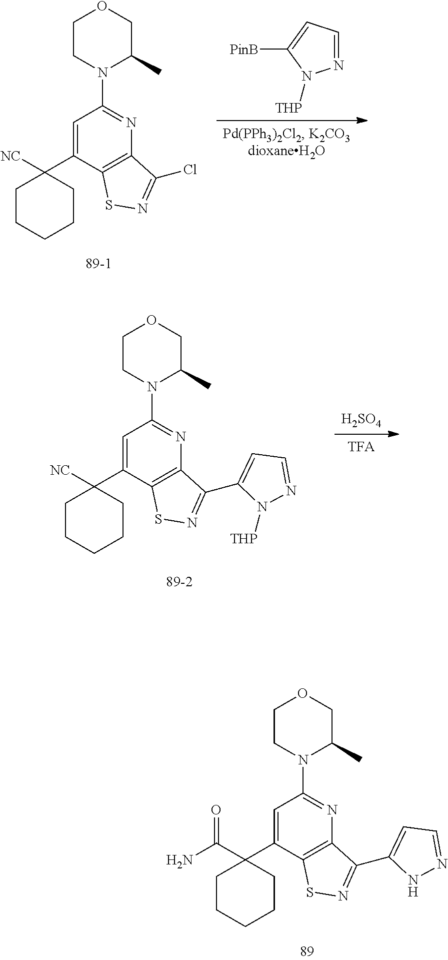

### Step 1. 1-{5-[(3R)-3-methylmorpholin-4-yl]-3-[1-(oxan-2-yl)-1H-pyrazol-5-yl]-[1,2]thiazolo[4,5-b]pyridin-7-yl}cyclohexane-1-carbonitrile

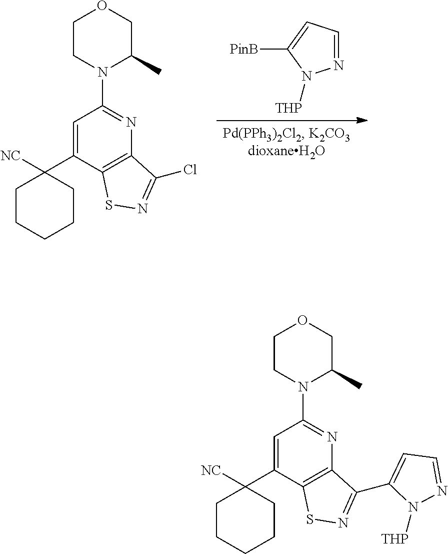

A mixture of 1-{3-chloro-5-[(3R)-3-methylmorpholin-4-yl]-[1,2]thiazolo[4,5-b]pyridin-7-yl}cyclohexane-1-carbonitrile (84 mg, 0.223 mmol), 1-(oxan-2-yl)-5-(tetramethyl-1,3,2-dioxaborolan-2-yl)-1H-pyrazole (123.98 mg, 0.446 mmol), Pd(dppf)Cl₂ (32.61 mg, 0.045 mmol) and K₂CO₃ (110.57 mg, 0.8 mmol) in dioxane (2 mL) and water (0.4 mL) was stirred overnight at 100° C. under nitrogen atmosphere. The reaction mixture was diluted with water and extracted with ethyl acetate. The combined organic layer was washed with brine, dried over Na₂SO₄, filtered and concentrated in vacuo. The residue was purified on flash column chromatography (Silica, 0˜60% Ethyl Acetate in petroleum ether) to give the title product 1-{5-[(3R)-3-methylmorpholin-4-yl]-3-[1-(oxan-2-yl)-1H-pyrazol-5-yl]-[1,2]thiazolo[4,5-b]pyridin-7-yl}cyclohexane-1-carbonitrile (70 mg, 0.142 mmol, 63.76%). LC-MS(ESI+): m/z (M+H)=492.8

### Step 2. 1-{5-[(3R)-3-methylmorpholin-4-yl]-3-(1H-pyrazol-5-yl)-[1,2]thiazolo[4,5-b]pyridin-7-yl}cyclohexane-1-carboxamide

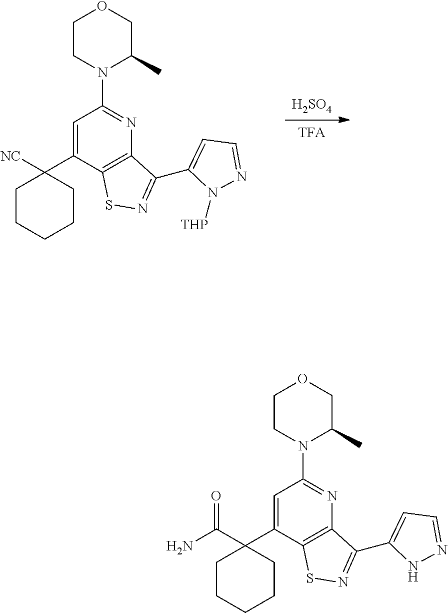

To a solution of 1-{5-[(3R)-3-methylmorpholin-4-yl]-3-[1-(oxan-2-yl)-1H-pyrazol-5-yl]-[1,2]thiazolo[4,5-b]pyridin-7-yl}cyclohexane-1-carbonitrile (70 mg, 0.142 mmol) in TFA (3.5 mL) was added H₂SO₄ (0.5 mL) and the resulting mixture was stirred for 2 hrs at 100° C. under nitrogen atmosphere. The mixture was concentrated and basified with saturated ammonium. The mixture was extracted with ethyl acetate and the organic layer was washed with brine, dried over Na₂SO₄, filtered and concentrated. The residue was purified on Prep-HPLC (C18, 10-95%, acetonitrile in water with 0.1% formic acid) to give the title product 1-{5-[(3R)-3-methylmorpholin-4-yl]-3-(1H-pyrazol-5-yl)-[1,2]thiazolo[4,5-b]pyridin-7-yl}cyclohexane-1-carboxamide (22.4 mg, 0.053 mmol, 36.96%). LC-MS(ESI+): m/z (M+H)=426.9. ¹H NMR (400 MHz, DMSO) δ 13.60 (br, 1H), 7.68 (s, 1H), 7.36 (d, J=1.7 Hz, 1H), 7.18 (d, J=17.6 Hz, 2H), 7.08 (s, 1H), 4.52 (d, J=6.0 Hz, 1H), 4.07 (t, J=13.0 Hz, 2H), 3.83 (d, J=11.2 Hz, 1H), 3.72 (dd, J=11.4, 2.7 Hz, 1H), 3.56 (dt, J=11.6, 5.9 Hz, 1H), 3.30-3.23 (m, 1H), 2.58-2.53 (m, 2H), 1.85-1.75 (m, 2H), 1.66-1.54 (m, 5H), 1.35-1.28 (m, 1H), 1.26 (d, J=6.6 Hz, 3H).

### Example 90

### Synthesis of 1-{5-[(3R)-3-methylmorpholin-4-yl]-3-(1H-pyrazol-5-yl)-[1,2]thiazolo[4,5-b]pyridin-7-yl}cyclopentane-1-carboxamide

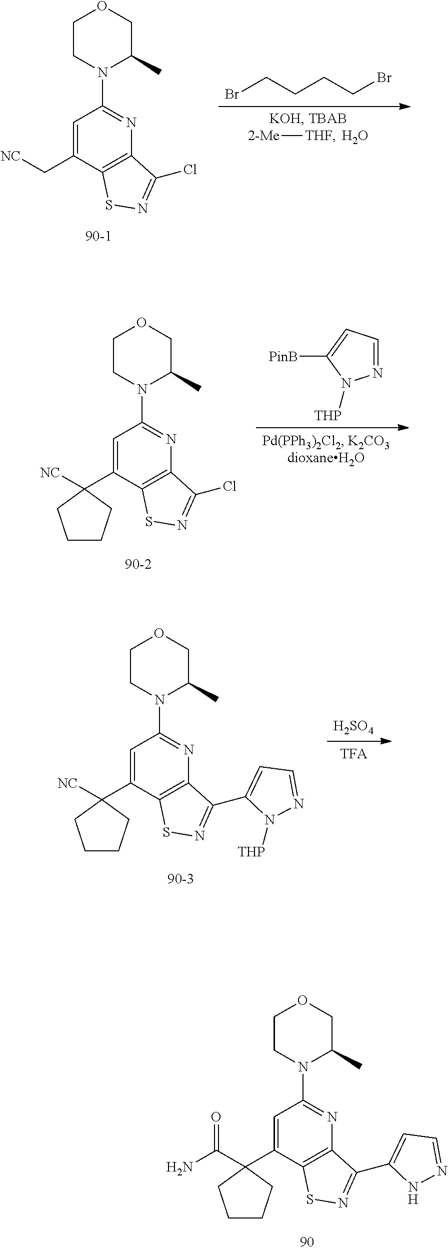

### Step 1. 1-{3-chloro-5-[(3R)-3-methylmorpholin-4-yl]-[1,2]thiazolo[4,5-b]pyridin-7-yl}cyclopentane-1-carbonitrile

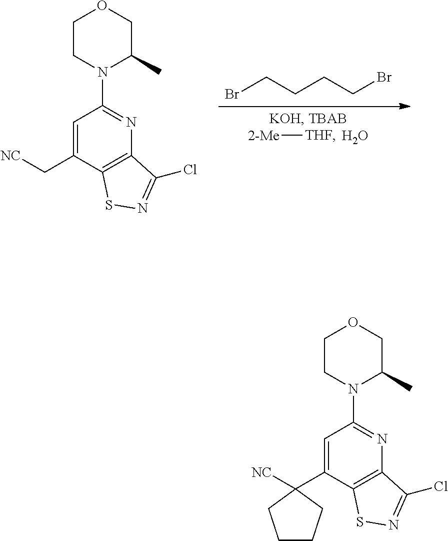

A mixture of 2-{3-chloro-5-[(3R)-3-methylmorpholin-4-yl]-[1,2]thiazolo[4,5-b]pyridin-7-yl}acetonitrile (100 mg, 0.324 mmol), 1,4-dibromobutane (0.155 mL, 1.295 mmol), KOH (363.42 mg, 6.477 mmol) and TBAB (0.020 mL, 0.065 mmol) in 2-Methyltetrahydrofuran (10 mL) and water (1 mL) was stirred at 80° C. for 4 hrs under nitrogen atmosphere. The reaction mixture was diluted with water and extracted with ethyl acetate. The combined organic layer was washed with brine, dried over Na₂SO₄, filtered and concentrated in vacuo. The residue was purified on flash column chromatography (Silica, 0˜30% Ethyl Acetate in petroleum ether) to give the title product 1-{3-chloro-5-[(3R)-3-methylmorpholin-4-yl]-[1,2]thiazolo[4,5-b]pyridin-7-yl}cyclopentane-1-carbonitrile (92 mg, 0.254 mmol, 78.28%). LC-MS(ESI+): m/z (M+H)=362.8, 364.9

### Step 2. 1-{5-[(3R)-3-methylmorpholin-4-yl]-3-[1-(oxan-2-yl)-1H-pyrazol-5-yl]-[1,2]thiazolo[4,5-b]pyridin-7-yl}cyclopentane-1-carbonitrile

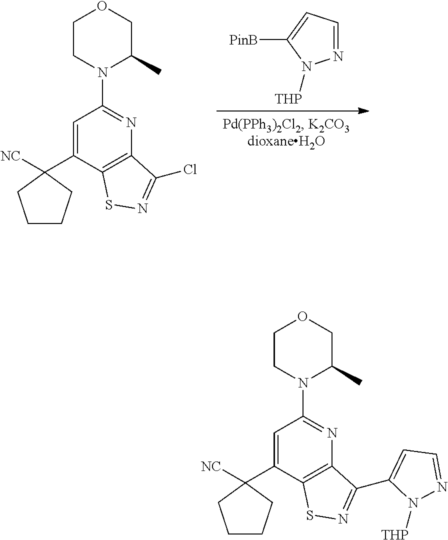

A mixture of 1-{3-chloro-5-[(3R)-3-methylmorpholin-4-yl]-thiazolo[4,5-b]pyridin-7-yl}cyclopentane-1-carbonitrile (92 mg, 0.254 mmol), 1-(oxan-2-yl)-5-(tetramethyl-1,3,2-dioxaborolan-2-yl)-1H-pyrazole (141.04 mg, 0.507 mmol), Pd(dppf)Cl₂ (37.10 mg, 0.051 mmol) and K₂CO₃ (110.57 mg, 0.8 mmol) in dioxane (2 mL) and water (0.4 mL) was stirred overnight at 100° C. under nitrogen atmosphere. The reaction mixture was diluted with water and extracted with Ethyl Acetate. The combined organic layer was washed with brine, dried over Na₂SO₄, filtered and concentrated in vacuo. The residue was purified on flash column chromatography (Silica, 0˜60% Ethyl Acetate in petroleum ether) to give the title product 1-{5-[(3R)-3-methylmorpholin-4-yl]-3-[1-(oxan-2-yl)-1H-pyrazol-5-yl]-[1,2]thiazolo[4,5-b]pyridin-7-yl}cyclopentane-1-carbonitrile (85 mg, 0.178 mmol, 70.05%). LC-MS(ESI+): m/z (M+H)=478.8

### Step 3. 1-{5-[(3R)-3-methylmorpholin-4-yl]-3-(1H-pyrazol-5-yl)-[1,2]thiazolo[4,5-b]pyridin-7-yl}cyclopentane-1-carboxamide

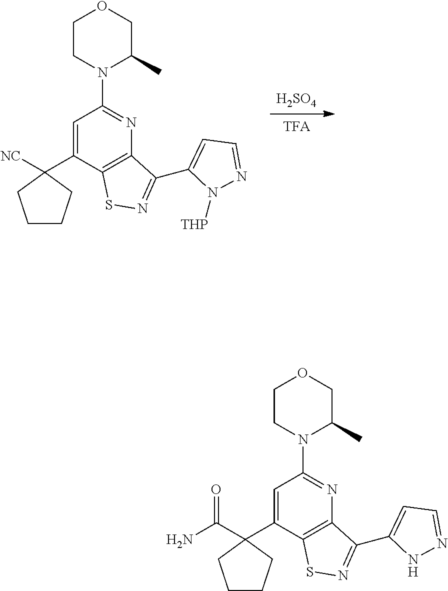

To a solution of 1-{5-[(3R)-3-methylmorpholin-4-yl]-3-[1-(oxan-2-yl)-1H-pyrazol-5-yl]-[1,2]thiazolo[4,5-b]pyridin-7-yl}cyclopentane-1-carbonitrile (40 mg, 0.084 mmol) in TFA (3.5 mL) was added H₂SO₄ (0.5 mL) and the resulting mixture was stirred for 2 hrs at 100° C. under nitrogen atmosphere. The mixture was concentrated and basified with saturated ammonium. The mixture was extracted with Ethyl Acetate and the organic layer was washed with brine, dried over Na₂SO₄, filtered and concentrated. The residue was purified on prep-HPLC (C18, 20-95%, MeOH in water with 0.1% formic acid) to give the title product 1-{5-[(3R)-3-methylmorpholin-4-yl]-3-(1H-pyrazol-5-yl)-[1,2]thiazolo[4,5-b]pyridin-7-yl}cyclopentane-1-carboxamide (15.6 mg, 0.038 mmol, 45.24%). LC-MS(ESI+): m/z (M+H)=412.9. ¹H NMR (400 MHz, DMSO) δ 13.57 (br, 1H), 7.72 (s, 1H), 7.37 (d, J=1.6 Hz, 1H), 7.17 (s, 1H), 7.08 (s, 2H), 4.54 (d, J=5.9 Hz, 1H), 4.13-4.03 (m, 2H), 3.83 (d, J=11.3 Hz, 1H), 3.74-3.69 (m, 1H), 3.57 (dd, J=11.6, 9.2 Hz, 1H), 3.28-3.24 (m, 1H), 2.67-2.58 (m, 2H), 2.04-1.93 (m, 2H), 1.72-1.66 (m, 4H), 1.26 (d, J=6.6 Hz, 3H).

### Example 91

### Synthesis of (R)-1-(5-(3-methylmorpholino)-3-(1H-pyrazol-5-yl)isothiazolo[4,5-b]pyridin-7-yl)cyclohexan-1-ol

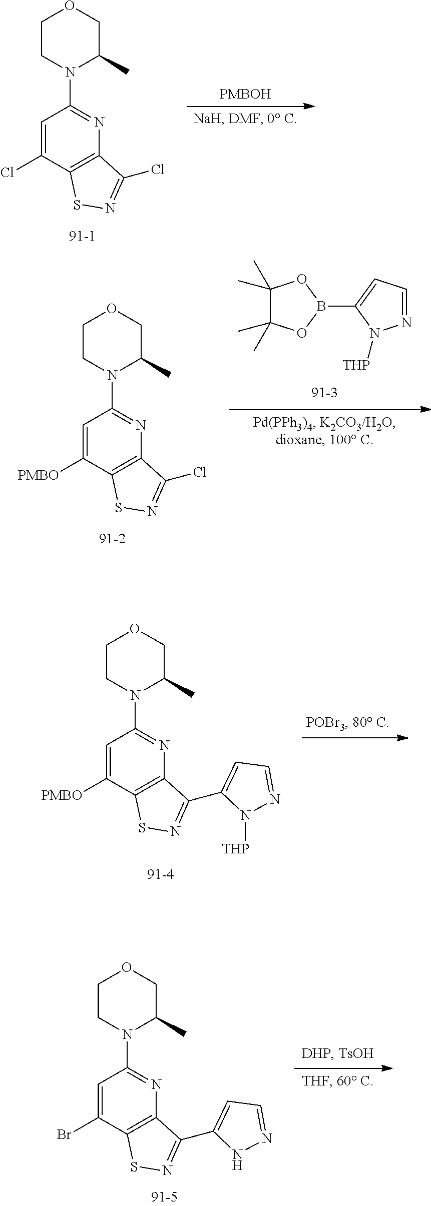

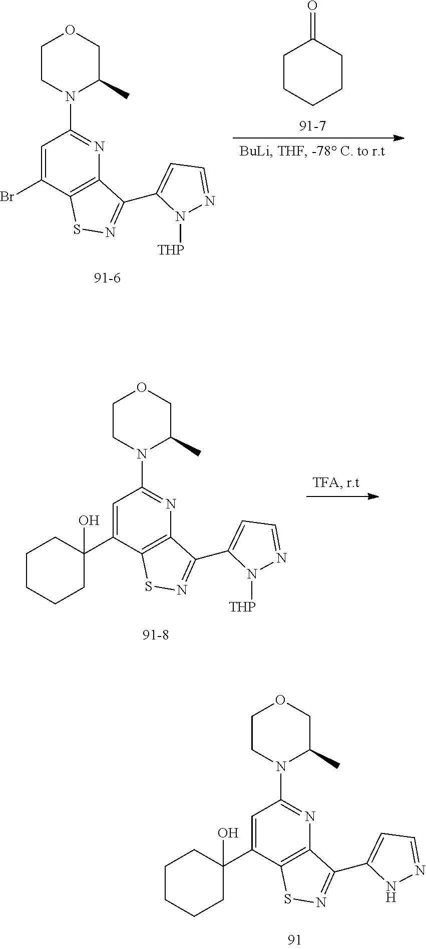

### Step 1. (R)-4-(3-chloro-7-((4-methoxybenzyl)oxy)isothiazolo[4,5-b]pyridin-5-yl)-3-methylmorpholine

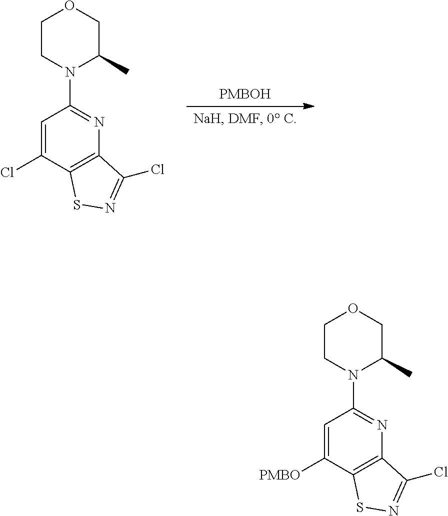

To a solution of NaH (dispersion in paraffin liquid, 60% w, 0.4 g, 9.90 mmol) in anhydrous DMF (15 mL) was added a solution of (4-methoxyphenyl)methanol (1.0 g, 7.23 mmol) in anhydrous DMF (5 mL) slowly. The resulting mixture was stirred at 0° C. for 15 min. Then (3R)-4-{3,7-dichloro-[1,2]thiazolo[4,5-b]pyridin-5-yl}-3-methylmorpholine (2.0 g, 6.57 mmol) was added to the mixture in one portion. The resulting mixture was stirred at 0° C. for 1 h. The reaction mixture was quenched with NaHCO₃ aqueous solution. The mixture was extracted with EA and the combined organic layer was dried over Na₂SO₄, filtered and concentrated in vacuo. The residue was purified on flash column eluting with PE:EA=2:1 to afford the desired product (1.18 g, yield: 44%). LC/MS (ESI): m/z 406 [M+H]⁺.

### Step 2. (3R)-4-(7-((4-methoxybenzyl)oxy)-3-(1-(tetrahydro-2H-pyran-2-yl)-1H-pyrazol-5-yl)isothiazolo[4,5-b]pyridin-5-yl)-3-methylmorpholine

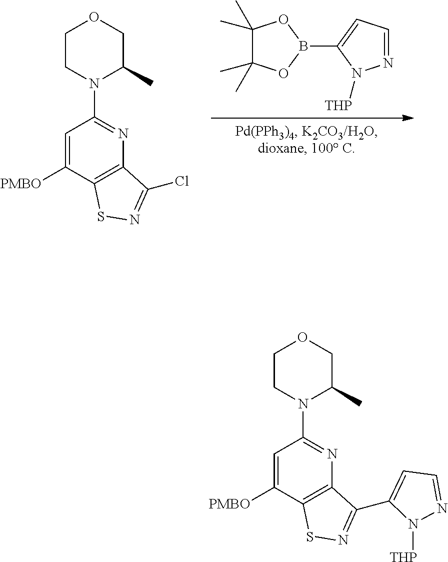

A mixture of (3R)-4-{3-chloro-7-[(4-methoxyphenyl)methoxy]-[1,2]thiazolo[4,5-b]pyridin-5-yl}-3-methylmorpholine (500 mg, 1.23 mmol), 1-(oxan-2-yl)-5-(tetramethyl-1,3,2-dioxaborolan-2-yl)-1H-pyrazole (1.02 g, 3.69 mmol), Pd(PPh₃)₄ (284 mg, 0.24 mmol) and K₂CO₃ (2.0 M in H₂O, 3.0 mL, 6.16 mmol) in dioxane (15 mL) was stirred at 100° C. for 16 h under N₂ atmosphere. LC-MS showed the reaction was complete. The reaction mixture was diluted with H₂O (20 mL), then extracted with EA (50 mL×3). The combined organic layer was washed with brine, dried over anhydrous Na₂SO₄, filtered and concentrated. The residue was purified by column chromatography on silica gel (PE:EA=2:1, V/V) to give the desired product (200 mg, yield: 31%). LC/MS (ESI): m/z 522 [M+H]⁺.

### Step 3. (R)-4-(7-bromo-3-(1H-pyrazol-5-yl)isothiazolo[4,5-b]pyridin-5-yl)-3-methylmorpholine

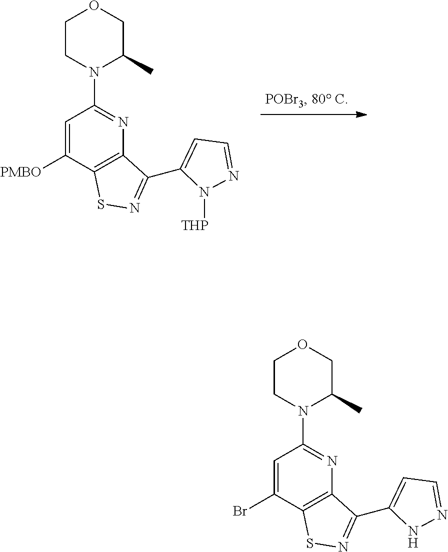

A mixture of (3R)-4-{7-[(4-methoxyphenyl)methoxy]-3-[1-(oxan-2-yl)-1H-pyrazol-5-yl]-[1,2]thiazolo[4,5-b]pyridin-5-yl}-3-methylmorpholine (200 mg, 0.38 mmol) and POBr₃ (500 mg, 1.74 mmol) was stirred at 80° C. under N₂ atmosphere for 3 h. The reaction mixture was diluted with DCM and washed with H₂O. The organic layer was dried over Na₂SO₄, filtered and concentrated in vacuo. The residue was purified on flash column eluting with DCM:MeOH=20:1 to give the desired product (64 mg, yield: 43%). LC/MS (ESI): m/z 380 [M+H]⁺.

### Step 4. (3R)-4-(7-bromo-3-(1-(tetrahydro-2H-pyran-2-yl)-1H-pyrazol-5-yl)isothiazolo[4,5-b]pyridin-5-yl)-3-methylmorpholine

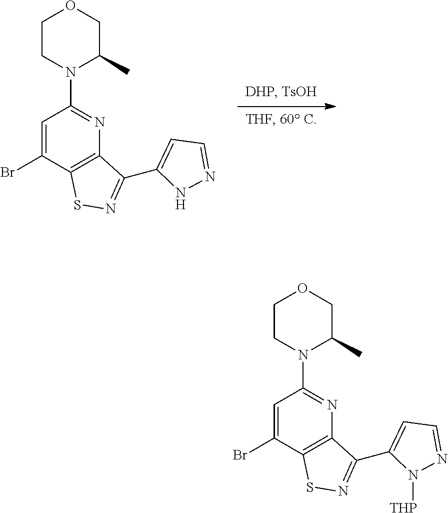

A mixture of (3R)-4-[7-bromo-3-(1H-pyrazol-5-yl)-[1,2]thiazolo[4,5-b]pyridin-5-yl]-3-methylmorpholine (64 mg, 0.16 mmol), 3,4-Dihydro-2H-pyran (63 mg, 0.75 mmol) and TsOH (5 mg, 0.03 mmol) in THF (3 mL) was stirred at 65° C. for 16 h. LC-MS showed the reaction was complete. The reaction mixture was diluted with EA (40 mL), then washed with water and brine, dried over anhydrous Na₂SO₄, filtered and concentrated. The residue was purified by column chromatography on silica gel (PE:EA=2:1, V/V) to afford the desired product (64 mg, yield: 81%). LC/MS (ESI): m/z 464 [M+H]⁺.

### Step 5. 1-(5-((R)-3-methylmorpholino)-3-(1-(tetrahydro-2H-pyran-2-yl)-1H-pyrazol-5-yl)isothiazolo[4,5-b]pyridin-7-yl)cyclohexan-1-ol

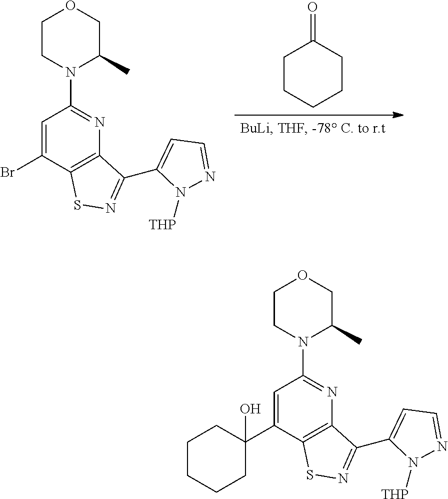

To a solution of (3R)-4-{7-bromo-3-[1-(oxan-2-yl)-1H-pyrazol-5-yl]-[1,2]thiazolo[4,5-b]pyridin-5-yl}-3-methylmorpholine (64 mg, 0.13 mmol) and cyclohexanone (40 mg, 0.41 mmol) in anhydrous THF (2 mL) was added n-BuLi (2.5 M in hexane, 0.16 mL, 0.41 mmol) slowly. The resulting mixture was stirred at −78° C. under N₂ atmosphere for 2 h. LCMS showed the reaction was completed. The reaction mixture was quenched with NaHCO₃ aqueous solution and extracted with EA. The combined organic layer was dried over Na₂SO₄, filtered and concentrated in vacuum. The residue was purified by column chromatography on silica gel (PE:EA=2:1, V/V) to afford the desired product (32 mg, yield: 48%). LC/MS (ESI): m/z 484 [M+H]⁺.

### Step 6. (R)-1-(5-(3-methylmorpholino)-3-(1H-pyrazol-5-yl)isothiazolo[4,5-b]pyridin-7-yl)cyclohexan-1-ol

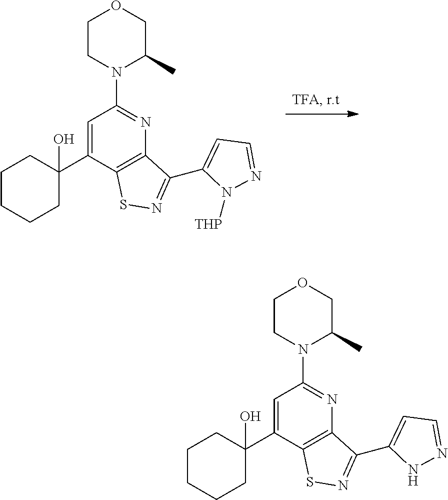

A mixture of 1-{5-[(3R)-3-methylmorpholin-4-yl]-3-[1-(oxan-2-yl)-1H-pyrazol-5-yl]-[1,2]thiazolo[4,5-b]pyridin-7-yl}cyclohexan-1-ol (30 mg, 0.06 mmol) in DCM/TFA (V/V, 1 mL/1 mL) was stirred at room temperature for 16 h. After concentration, the residue was purified by prep-HPLC (C18, 10-95%, MeOH in H₂O with 0.1% HCOOH) to give the desired product (5 mg, yield: 20%). LC/MS (ESI): m/z 400 [M+H]⁺. 1H NMR (400 MHz, DMSO-d6) δ 13.46 (s, 1H), 7.70 (s, 1H), 7.36 (s, 1H), 7.06 (s, 1H), 5.85 (s, 1H), 4.56 (s, 1H), 4.06 (dd, J=33.0, 11.4 Hz, 2H), 3.76 (dd, J=36.6, 10.5 Hz, 2H), 3.56 (t, J=10.8 Hz, 1H), 3.21 (d, J=11.6 Hz, 1H), 1.81 (dd, J=36.6, 11.9 Hz, 6H), 1.58 (s, 2H), 1.36 (d, J=10.6 Hz, 1H), 1.25-1.12 (m, 4H).

### Example 92

### Synthesis of 1-{5-[(3R)-3-methylmorpholin-4-yl]-3-(1H-pyrazol-5-yl)-[1,2]thiazolo[4,5-b]pyridin-7-yl}cyclopentane-1-carboxylate

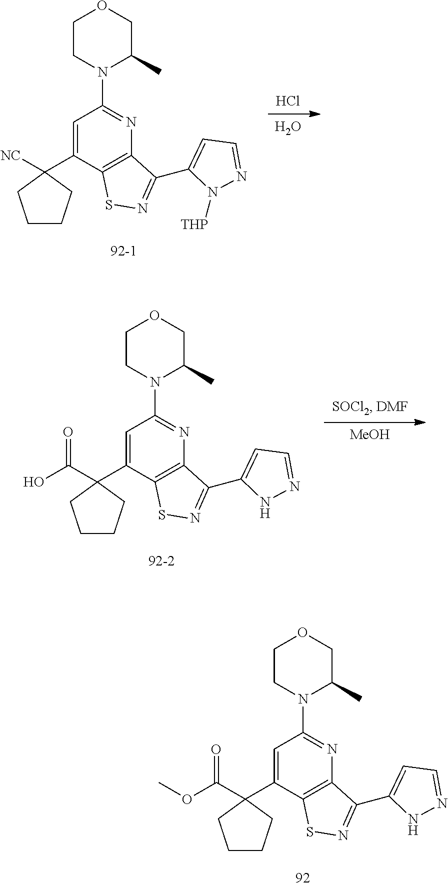

### Step 1. 1-{5-[(3R)-3-methylmorpholin-4-yl]-3-(1H-pyrazol-5-yl)-[1,2]thiazolo[4,5-b]pyridin-7-yl}cyclopentane-1-carboxylic acid

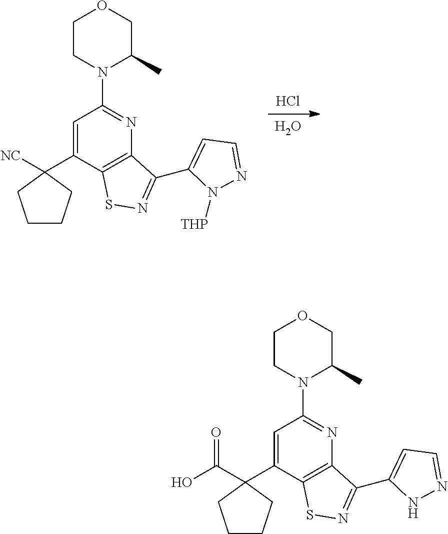

A solution of 1-{5-[(3R)-3-methylmorpholin-4-yl]-3-[1-(oxan-2-yl)-1H-pyrazol-5-yl]-[1,2]thiazolo[4,5-b]pyridin-7-yl}cyclopentane-1-carbonitrile (67 mg, 0.140 mmol) in HCl (12 mL, 144.000 mmol, 37% in water) was stirred overnight at 100° C. under nitrogen atmosphere. After concentrated in vacuo, the residue was azeotroped with toluene twice to give the title product 1-{5-[(3R)-3-methylmorpholin-4-yl]-3-(1H-pyrazol-5-yl)-[1,2]thiazolo[4,5-b]pyridin-7-yl}cyclopentane-1-carboxylic acid (57 mg, 0.138 mmol, 98.48%) and used in next step without further purification. LC-MS(ESI+): m/z (M+H)=413.9.

### Step 2. 1-{5-[(3R)-3-methylmorpholin-4-yl]-3-(1H-pyrazol-5-yl)-[1,2]thiazolo[4,5-b]pyridin-7-yl}cyclopentane-1-carboxylate

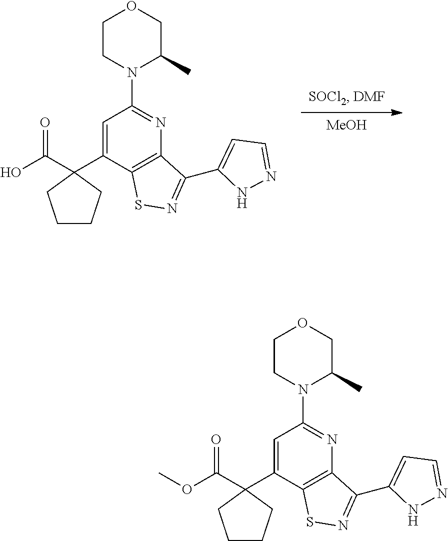

To an ice-cooled solution of 1-{5-[(3R)-3-methylmorpholin-4-yl]-3-(1H-pyrazol-5-yl)-[1,2]thiazolo[4,5-b]pyridin-7-yl}cyclopentane-1-carboxylic acid (57 mg, 0.138 mmol) and DMF (0.05 mL, 0.646 mmol) in MeOH (10 mL) was added SOCl₂ (1 mL, 13.785 mmol) dropwise and the resulting mixture was stirred for 2 hrs at 60° C. under nitrogen atmosphere. The mixture was concentrated and basified with saturated NaHCO₃ and extracted with ethyl acetate. The organic layer was washed with brine, dried over Na₂SO₄, filtered and concentrated. The residue was purified on prep-HPLC (C18, 20-95%, acetonitrile in water with 0.1% formic acid) to give the title product methyl1-{5-[(3R)-3-methylmorpholin-4-yl]-3-(1H-pyrazol-5-yl)-[1,2]thiazolo[4,5-b]pyridin-7-yl}cyclopentane-1-carboxylate (18.4 mg, 0.043 mmol, 31.22%). LC-MS(ESI+): m/z (M+H)=427.9. ¹H NMR (400 MHz, DMSO) δ 7.74 (s, 1H), 7.37 (d, J=1.8 Hz, 1H), 7.09 (s, 1H), 4.61-4.53 (m, 1H), 4.13 (d, J=12.6 Hz, 1H), 4.06-4.00 (m, 1H), 3.81 (d, J=11.3 Hz, 1H), 3.74-3.70 (m, 1H), 3.58 (s, 3H), 3.57-3.53 (m, 1H), 3.28-3.22 (m, 1H), 2.63-2.56 (m, 2H), 2.22-2.10 (m, 2H), 1.80-1.71 (m, 4H), 1.23 (d, J=6.6 Hz, 3H).

### Example 93 Synthesis of (3R)-3-methyl-4-[3-(3-methyl-1H-1,2,4-triazol-5-yl)-7-(1-methyl-1H-pyrazol-5-yl)-[1,2]thiazolo[4,5-b]pyridin-5-yl]morpholine

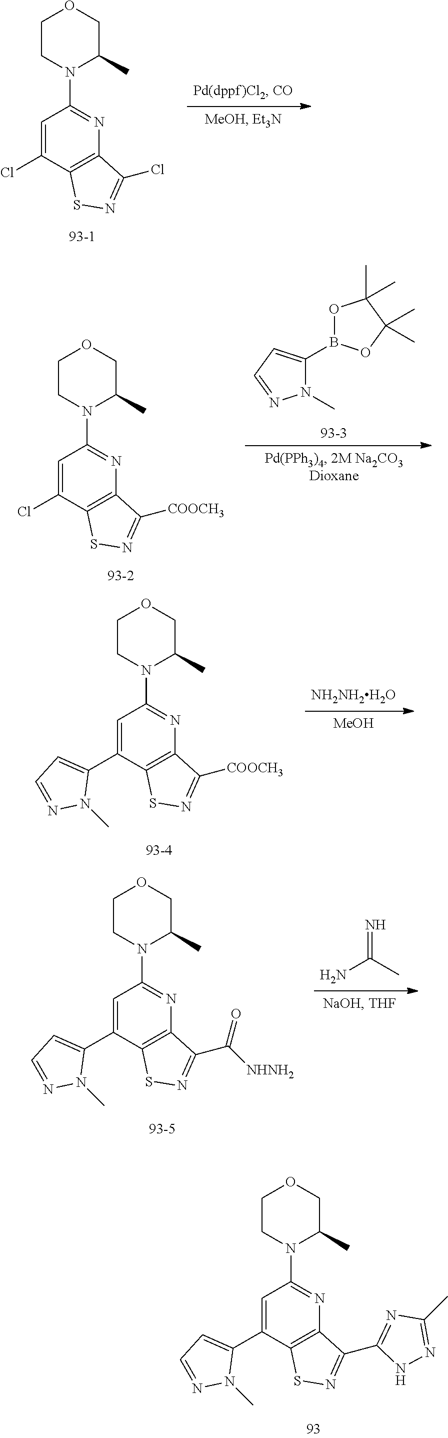

### Step 1. methyl 7-chloro-5-[(3R)-3-methylmorpholin-4-yl]-[1,2]thiazolo[4,5-b]pyridine-3-carboxylate

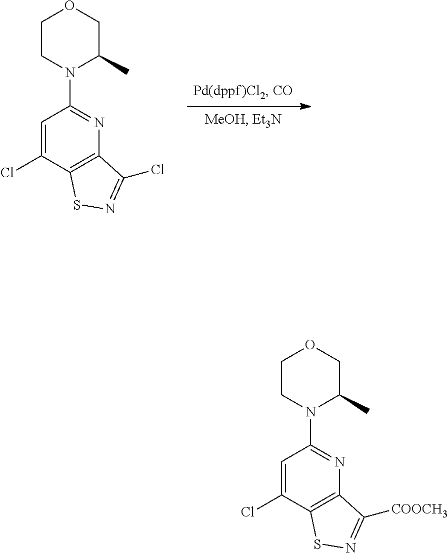

To a solution of (3R)-4-{3,7-dichloro-[1,2]thiazolo[4,5-b]pyridin-5-yl}-3-methylmorpholine (500 mg, 1.644 mmol) in MeOH (25 mL) was added Pd(dppf)Cl₂ (360.80 mg, 0.493 mmol) and TEA (2.285 mL, 16.437 mmol), and the reaction was stirred at 60° C. for overnight under CO atmosphere. The reaction was diluted with EA and water. The organic layer was separated, washed with further saturated NaCl solution, and concentrated in vacuo. The residue was purified via Biotage (PE:EA=5:1) to afford the methyl 7-chloro-5-[(3R)-3-methylmorpholin-4-yl]-[1,2]thiazolo[4,5-b]pyridine-3-carboxylate (175 mg, 0.534 mmol, 32.48%). LC/MS (ESI) m/z:328 (M+H)⁺.

### Step 2. methyl 7-(1-methyl-1H-pyrazol-5-yl)-5-[(3R)-3-methylmorpholin-4-yl]-[1,2]thiazolo[4,5-b]pyridine-3-carboxylate

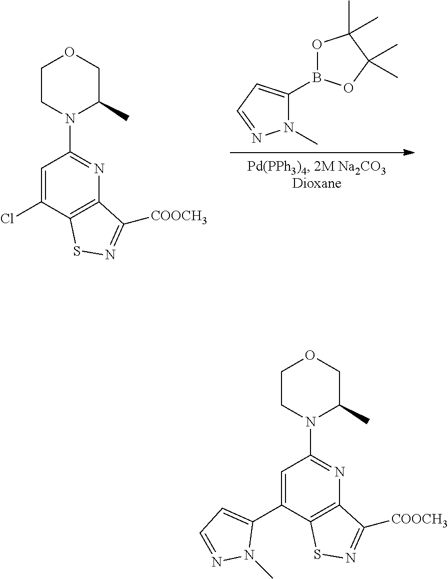

To a solution of methyl 7-chloro-5-[(3R)-3-methylmorpholin-4-yl]-[1,2]thiazolo[4,5-b]pyridine-3-carboxylate (175 mg, 0.534 mmol) in dioxane (10 mL) was added 1-methyl-5-(tetramethyl-1,3,2-dioxaborolan-2-yl)-1H-pyrazole (333.25 mg, 1.602 mmol), Pd(dppf)Cl₂ (39.06 mg, 0.053 mmol) and K₂CO₃ (147.57 mg, 1.068 mmol). The reaction was stirred at 100° C. overnight under nitrogen atmosphere. The reaction was diluted with EA and water. The organic layer was separated, washed with further saturated NaCl, and concentrated in vacuo to give the title product methyl 7-(1-methyl-1H-pyrazol-5-yl)-5-[(3R)-3-methylmorpholin-4-yl]-[1,2]thiazolo[4,5-b]pyridine-3-carboxylate (130 mg, 0.348 mmol, 65.20%). LC/MS (ESI) m/z:374 (M+H)⁺.

### Step 3. 7-(1-methyl-1H-pyrazol-5-yl)-5-[(3R)-3-methylmorpholin-4-yl]-[1,2]thiazolo[4,5-b]pyridine-3-carbohydrazide

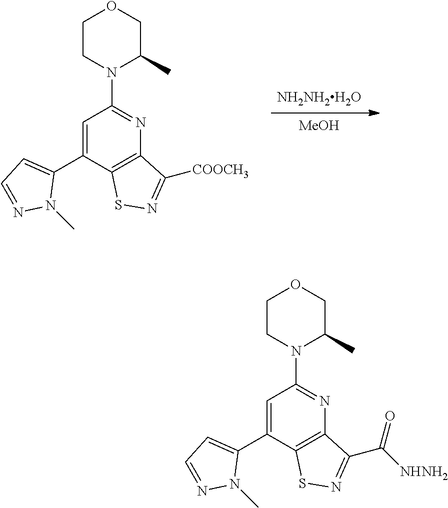 [SMILES: "MeOH" ; COC(=O)c1nsc2c(-c3ccnn3C)cc(N3CCOC[C@H]3C)nc12 ; "NH2NH2·H2O" ; C[C@@H]1COCCN1c1cc(-c2ccnn2C)c2snc(C(=O)NN)c2n1]

To a solution of methyl 7-(1-methyl-1H-pyrazol-5-yl)-5-[(3R)-3-methylmorpholin-4-yl]-[1,2]thiazolo[4,5-b]pyridine-3-carboxylate (100 mg, 0.268 mmol) in MeOH (10 mL) were added NH₂NH₂·H₂O (1 mL), and the reaction was stirred at 80° C. overnight. The reaction mixture was extracted with ethyl acetate, washed with H₂O and brine, dried over Na₂SO₄, filtered and concentrated in vacuo to give the title product 7-(1-methyl-1H-pyrazol-5-yl)-5-[(3R)-3-methylmorpholin-4-yl]-thiazolo[4,5-b]pyridine-3-carbohydrazide (100 mg, 0.268 mmol, 100.00%). LC/MS (ESI) m/z:374 (M+H)⁺.

### Step 4. (3R)-3-methyl-4-[3-(3-methyl-1H-1,2,4-triazol-5-yl)-7-(1-methyl-1H-pyrazol-5-yl)-[1,2]thiazolo[4,5-b]pyridin-5-yl]morpholine

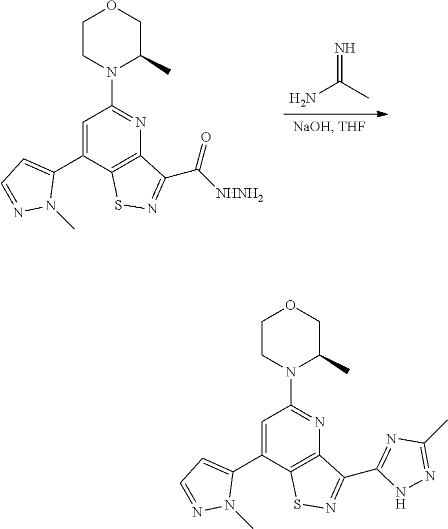

To a solution of 7-(1-methyl-1H-pyrazol-5-yl)-5-[(3R)-3-methylmorpholin-4-yl]-[1,2]thiazolo[4,5-b]pyridine-3-carbohydrazide (100 mg, 0.268 mmol) in MeOH (10 mL) were added ethanimidamide (31.11 mg, 0.536 mmol) and KOH (30.05 mg, 0.536 mmol), and the reaction was stirred at 80° C. for 4 hours. The reaction was diluted with EA and water. The organic layer was separated, washed with further saturated NaCl solution, and concentrated in vacuo. The residue was purified via Biotage (20:1; 10 g Cartridge column) to afford (3R)-3-methyl-4-[3-(3-methyl-1H-1,2,4-triazol-5-yl)-7-(1-methyl-1H-pyrazol-5-yl)-[1,2]thiazolo[4,5-b]pyridin-5-yl]morpholine (22 mg, 0.055 mmol, 20.72%). LC/MS (ESI) m/z:397 (M+H)^{+ 1}H NMR (400 MHz, DMSO) δ 7.69 (d, J=1.9 Hz, 1H), 7.39 (s, 1H), 6.80 (d, J=1.8 Hz, 1H), 4.60 (s, 1H), 4.30 (d, J=13.1 Hz, 1H), 4.02 (s, 1H), 4.00 (s, 3H), 3.79 (d, J=11.3 Hz, 1H), 3.71 (d, J=11.6 Hz, 1H), 3.55 (t, J=10.5 Hz, 1H), 3.28-3.11 (m, 1H), 2.44 (s, 3H), 1.23 (d, J=6.6 Hz, 3H).

### Example 94

### Synthesis of imino(methyl)(l-(3-(3-methyl-1H-pyrazol-5-yl)-5-((R)-3-methylmorpholino)isothiazolo[4,5-b]pyridin-7-yl)cyclopropyl)-16-sulfanone

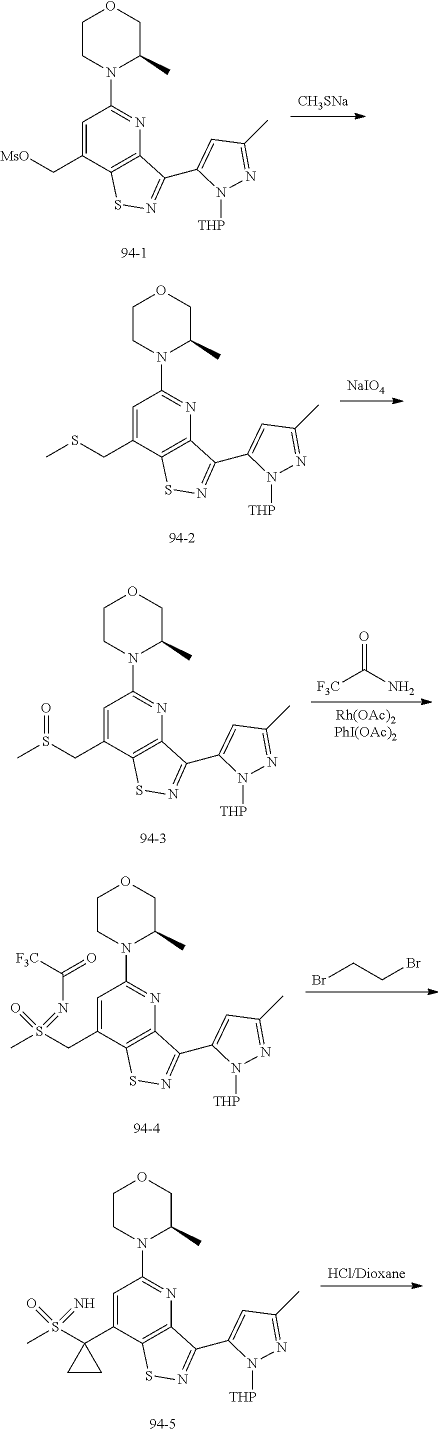

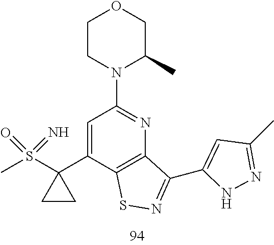

### Step 1. (3R)-3-methyl-4-(3-(3-methyl-1-(tetrahydro-2H-pyran-2-yl)-1H-pyrazol-5-yl)-7-((methylthio)methyl)isothiazolo[4,5-b]pyridin-5-yl)morpholine

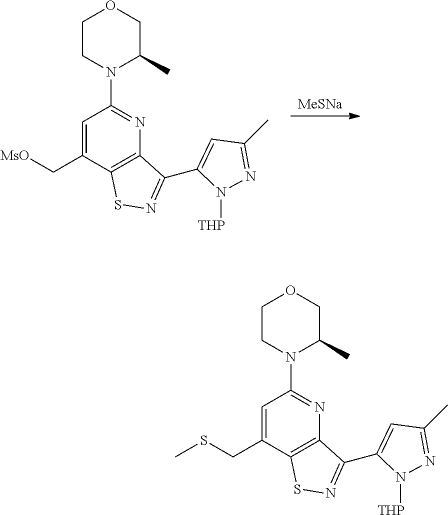

To a mixture of (3-(3-methyl-1-(tetrahydro-2H-pyran-2-yl)-1H-pyrazol-5-yl)-5-((R)-3-methylmorpholino)isothiazolo[4,5-b]pyridin-7-yl)methyl methanesulfonate (388 mg, 0.764 mmol) in DMF (10 mL) was added MeSNa (107 mg, 1.53 mmol). The mixture was stirred at rt for 2 hs. LC-MS showed the reaction was complete. The reaction mixture was poured into H₂O and extracted with EA (30 mL×3). The combined organic phase was washed brine, dried over Na₂SO₄, filtered and concentrated. The residue was purified by flash chromatography (silica gel (10 g), 0-100%, EA in PE) to give (3R)-3-methyl-4-(3-(3-methyl-1-(tetrahydro-2H-pyran-2-yl)-1H-pyrazol-5-yl)-7-((methylthio)methyl)isothiazolo[4,5-b]pyridin-5-yl)morpholine (231 mg, 0.503 mmol, 66%). LC/MS (ESI): m/z 460.7 [M+1]+.

### Step 2. (3R)-3-methyl-4-(3-(3-methyl-1-(tetrahydro-2H-pyran-2-yl)-1H-pyrazol-5-yl)-7-((methylsulfinyl)methyl)isothiazolo[4,5-b]pyridin-5-yl)morpholine

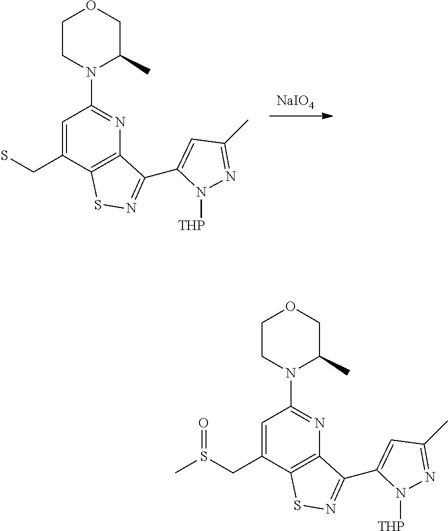

To a mixture of (3R)-3-methyl-4-(3-(3-methyl-1-(tetrahydro-2H-pyran-2-yl)-1H-pyrazol-5-yl)-7-((methylthio)methyl)isothiazolo[4,5-b]pyridin-5-yl)morpholine (231 mg, 0.503 mmol) in MeOH (10 mL) and H₂O (2 mL) was added NaIO4 (215 mg, 1.01 mmol). After the mixture was stirred at rt for 2 hs. LC-MS showed the reaction was complete. The reaction mixture was poured into H₂O and extracted with DCM (30 mL*3). The combined organic phase was washed brine, dried over Na₂SO₄, filtered and concentrated. The residue was purified by flash chromatography (silica gel (10 g), 0-100%, MeOH in DCM) to give (3R)-3-methyl-4-(3-(3-methyl-1-(tetrahydro-2H-pyran-2-yl)-1H-pyrazol-5-yl)-7-((methylsulfinyl)methyl)isothiazolo[4,5-b]pyridin-5-yl)morpholine (221 mg, 0.465 mmol, 92%). LC/MS (ESI): m/z 476.7 [M+H]⁺.

### Step 3. 2,2,2-trifluoro-N-(methyl((3-(3-methyl-1-(tetrahydro-2H-pyran-2-yl)-1H-pyrazol-5-yl)-5-((R)-3-methylmorpholino)isothiazolo[4,5-b]pyridin-7-yl)methyl)(oxo)-16-sulfanylidene)acetamide

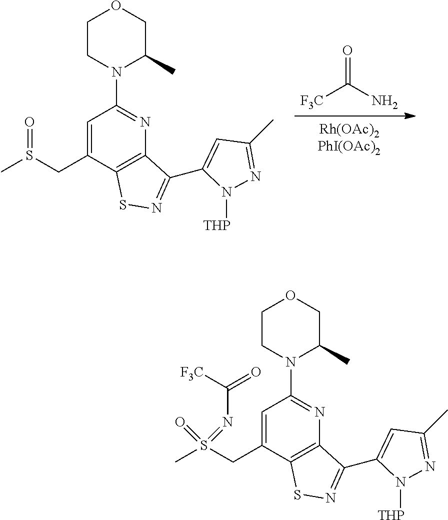

To a mixture of (3R)-3-methyl-4-(3-(3-methyl-1-(tetrahydro-2H-pyran-2-yl)-1H-pyrazol-5-yl)-7-((methylsulfinyl)methyl)isothiazolo[4,5-b]pyridin-5-yl)morpholine (220 mg, 0.463 mmol), PhI(OAc)₂ (542 mg, 1.16 mmol) and trifluoroacetamide (78 mg, 0.694 mmol) in anisole (8 mL) was added Rh(OAc)₂ (21 mg, 0.093 mmol). After the mixture was stirred at 60° C. for 12 hs. LC-MS showed the reaction was complete. The mixture was filtered and concentrated to dryness. The residue was purified by flash chromatography (silica gel (4 g), 0-100%, EA in PE) to give 2,2,2-trifluoro-N-(methyl((3-(3-methyl-1-(tetrahydro-2H-pyran-2-yl)-1H-pyrazol-5-yl)-5-((R)-3-methylmorpholino)isothiazolo[4,5-b]pyridin-7-yl)methyl)(oxo)-16-sulfanylidene)acetamide (30 mg, 0.051 mmol, 11%). LC/MS (ESI): m/z 587.2 [M+H]⁺.

### Step 4. imino(methyl)(1-(3-(3-methyl-1-(tetrahydro-2H-pyran-2-yl)-1H-pyrazol-5-yl)-5-((R)-3-methylmorpholino)isothiazolo[4,5-b]pyridin-7-yl)cyclopropyl)-16-sulfanone

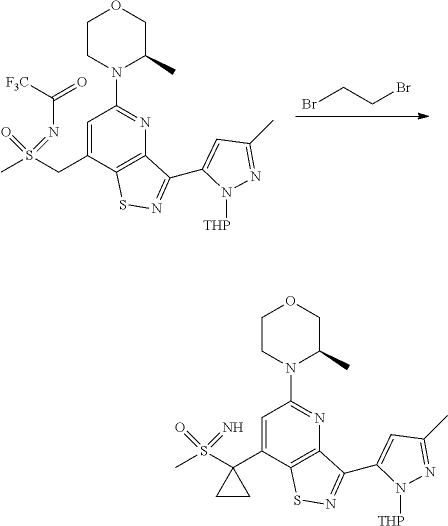

To a solution of 2,2,2-trifluoro-N-(methyl((3-(3-methyl-1-(tetrahydro-2H-pyran-2-yl)-1H-pyrazol-5-yl)-5-((R)-3-methylmorpholino)isothiazolo[4,5-b]pyridin-7-yl)methyl)(oxo)-16-sulfanylidene)acetamide (30 mg, 0.051 mmol), 1,2-dibromoethane (20 mg, 0.102 mmol) and TBAB (4 mg, 0.013 mmol) in Toluene (3 mL) was added NaOH (0.051 mL, 0.511 mmol, 10M in H₂O). After the mixture was stirred at 60° C. for 1 hr. LC-MS showed the reaction was complete. The reaction mixture was poured into H₂O and extracted with DCM (30 mL*3). The combined organic phase was washed brine, dried over anhydrous Na₂SO₄, filtered and concentrated. The residue was purified by flash chromatography (silica gel (4 g), 0-100%, EA in PE) to give imino(methyl)(1-(3-(3-methyl-1-(tetrahydro-2H-pyran-2-yl)-1H-pyrazol-5-yl)-5-((R)-3-methylmorpholino)isothiazolo[4,5-b]pyridin-7-yl)cyclopropyl)-16-sulfanone (6 mg, 0.012 mmol, 23%). LC/MS (ESI): m/z 517.2 [M+H]⁺.

### Step 5. imino(methyl)(1-(3-(3-methyl-1H-pyrazol-5-yl)-5-((R)-3-methylmorpholino)isothiazolo[4,5-b]pyridin-7-yl)cyclopropyl)-16-sulfanone

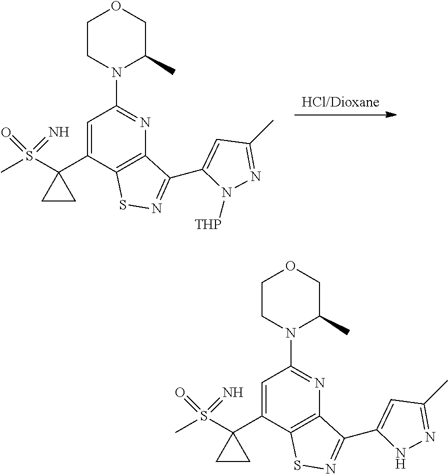

To a mixture of imino(methyl)(1-(3-(3-methyl-1-(tetrahydro-2H-pyran-2-yl)-1H-pyrazol-5-yl)-5-((R)-3-methylmorpholino)isothiazolo[4,5-b]pyridin-7-yl)cyclopropyl)-16-sulfanone (6 mg, 0.012 mmol) in DCM (0.5 mL) was added HCl/dioxane (1.5 mL, 4M). After the mixture was stirred at rt for 1 hr. LC-MS showed the reaction was complete. The mixture was concentrated to dryness. Then the crude product was purified by prep-HPLC (C₁₈, 10-95%, MeCN in H₂O with 0.1% HCOOH) to give imino(methyl)(1-(3-(3-methyl-1H-pyrazol-5-yl)-5-((R)-3-methylmorpholino)isothiazolo[4,5-b]pyridin-7-yl)cyclopropyl)-16-sulfanone (3 mg, 0.007 mmol, 60%). LC/MS (ESI): m/z 433.6 [M+H]⁺. ¹H NMR (400 MHz, DMSO-d6) S 13.18 (s, 1H), 7.46 (d, J=4.6 Hz, 1H), 7.10 (s, 1H), 4.59-4.43 (m, 1H), 4.18-4.09 (m, 1H), 4.08-3.95 (m, 2H), 3.82 (d, J=11.2 Hz, 1H), 3.72 (d, J=11.3 Hz, 1H), 3.57 (t, J=10.6 Hz, 1H), 3.28-3.17 (m, 2H), 2.90 (s, 3H), 2.30 (s, 3H), 1.85 (dt, J=10.6, 5.5 Hz, 1H), 1.58 (d, J=5.0 Hz, 1H), 1.45 (dd, J=17.8, 11.5 Hz, 1H), 1.39-1.28 (m, 1H), 1.25-1.21 (m, 3H).

### Example 95

### Synthesis of (3R)-4-[7-(2-methanesulfonylphenyl)-3-(3-methyl-1H-pyrazol-5-yl)-[1,2]thiazolo[4,5-b]pyridin-5-yl]-3-methylmorpholine

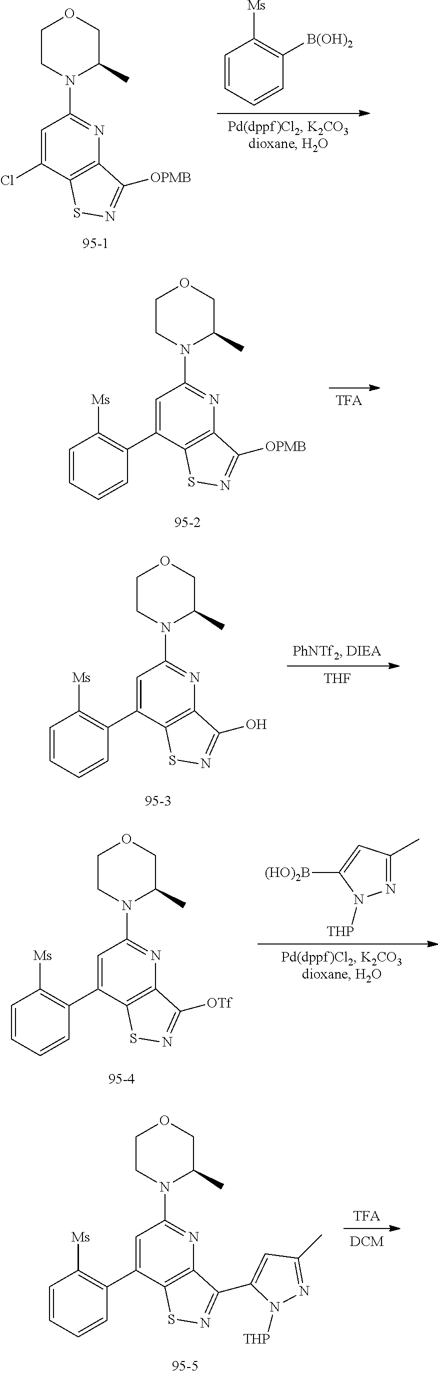

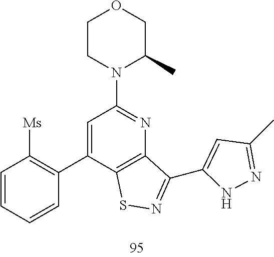 [SMILES: Cc1cc(-c2nsc3c(-c4ccccc4S(C)(=O)=O)cc(N4CCOC[C@H]4C)nc23)[nH]n1]

### Step 1. 1-{5-[(3R)-3-methylmorpholin-4-yl]-3-[1-(oxan-2-yl)-1H-pyrazol-5-yl]-[1,2]thiazolo[4,5-b]pyridin-7-yl}cyclopentane-1-carbonitrile

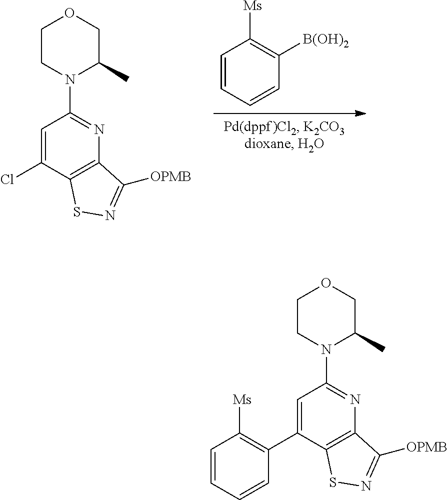

A mixture of (3R)-4-{7-chloro-3-[(4-methoxyphenyl)methoxy]-[1,2]thiazolo[4,5-b]pyridin-5-yl}-3-methylmorpholine (210 mg, 0.517 mmol), (2-methanesulfonylphenyl)boronic acid (206.96 mg, 1.035 mmol), Pd(dppf)Cl₂ (75.71 mg, 0.103 mmol) and K₂CO₃ (110.57 mg, 0.8 mmol) in dioxane (2 mL) and water (0.4 mL) was stirred overnight at 100° C. under nitrogen atmosphere. The reaction mixture was diluted with water and extracted with ethyl acetate. The combined organic layer was washed with brine, dried over Na₂SO₄, filtered and concentrated in vacuo. The residue was purified on flash column chromatography (Silica, 0˜60% Ethyl Acetate in petroleum ether) to give the title product (3R)-4-[7-(2-methanesulfonylphenyl)-3-[(4-methoxyphenyl)methoxy]-[1,2]thiazolo[4,5-b]pyridin-5-yl]-3-methylmorpholine (214 mg, 0.407 mmol, 78.69%). LC-MS(ESI+): m/z (M+H)=525.7

### Step 2. 7-(2-methanesulfonylphenyl)-5-[(3R)-3-methylmorpholin-4-yl]-thiazolo[4,5-b]pyridin-3-ol

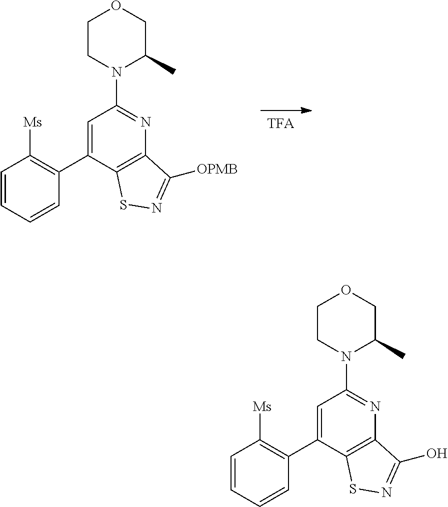

A solution of (3R)-4-[7-(2-methanesulfonylphenyl)-3-[(4-methoxyphenyl)methoxy]-[1,2]thiazolo[4,5-b]pyridin-5-yl]-3-methylmorpholine (214 mg, 0.407 mmol) in TFA (5 mL) was stirred at 70° C. for 1 h under nitrogen atmosphere. The reaction mixture was concentrated in vacuo to give the crude title product 7-(2-methanesulfonylphenyl)-5-[(3R)-3-methylmorpholin-4-yl]-[1,2]thiazolo[4,5-b]pyridin-3-ol (160 mg, 0.395 mmol, 96.92%). LC-MS(ESI+): m/z (M+H)=405.8

### Step 3. 7-(2-methanesulfonylphenyl)-5-[(3R)-3-methylmorpholin-4-yl]-[1,2]thiazolo[4,5-b]pyridin-3-yl trifluoromethanesulfonate

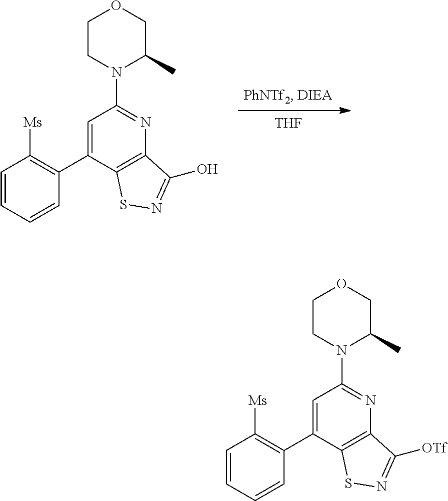 [SMILES: C[C@@H]1COCCN1c1cc(-c2ccccc2S(C)(=O)=O)c2snc(O)c2n1 ; "PhNTf2, DIEA" ; "THF" ; C[C@@H]1COCCN1c1cc(-c2ccccc2S(C)(=O)=O)c2snc(OS(=O)(=O)C(F)(F)F)c2n1]

A mixture of 7-(2-methanesulfonylphenyl)-5-[(3R)-3-methylmorpholin-4-yl]-[1,2]thiazolo[4,5-b]pyridin-3-ol (165 mg, 0.407 mmol), 1,1,1-trifluoro-N-phenyl-N-trifluoromethanesulfonylmethanesulfonamide (581.47 mg, 1.628 mmol) and DIEA (0.672 mL, 4.069 mmol) in THF (10 mL) was stirred at 70° C. for 2 hrs under nitrogen atmosphere. After diluted with water, the reaction mixture was extracted with ethyl acetate and the organic layer was washed with brine, dried over Na₂SO₄, filtered and concentrated. The residue was purified on flash column chromatography (Silica, 0˜60% Ethyl Acetate in petroleum ether) to give the title product 7-(2-methanesulfonylphenyl)-5-[(3R)-3-methylmorpholin-4-yl]-[1,2]thiazolo[4,5-b]pyridin-3-yl trifluoromethanesulfonate (48 mg, 0.089 mmol, 21.94%). LC-MS(ESI+): m/z (M+H)=537.8.

### Step 4. (3R)-4-[7-(2-methanesulfonylphenyl)-3-[3-methyl-1-(oxan-2-yl)-1H-pyrazol-5-yl]-[1,2]thiazolo[4,5-b]pyridin-5-yl]-3-methylmorpholine

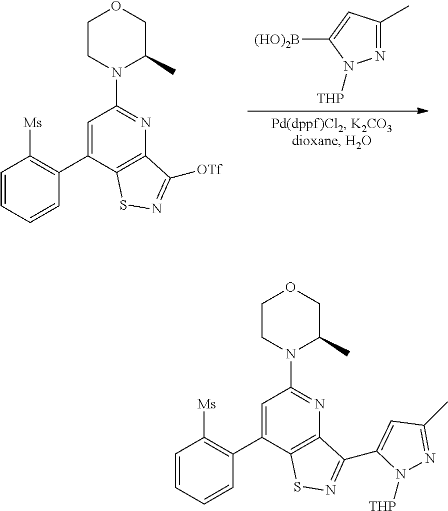

A mixture of 7-(2-methanesulfonylphenyl)-5-[(3R)-3-methylmorpholin-4-yl]-[1,2]thiazolo[4,5-b]pyridin-3-yl trifluoromethanesulfonate (42 mg, 0.078 mmol), [3-methyl-1-(oxan-2-yl)-1H-pyrazol-5-yl]boronic acid (49.23 mg, 0.234 mmol), Pd(dppf)Cl₂ (11.43 mg, 0.016 mmol) and K₂CO₃ (110.57 mg, 0.8 mmol) in dioxane (2 mL) and water (0.4 mL) was stirred overnight at 100° C. under nitrogen atmosphere. The reaction mixture was diluted with water and extracted with ethyl acetate. The combined organic layer was washed with brine, dried over Na₂SO₄, filtered and concentrated in vacuo. The residue was purified on flash column chromatography (Silica, 0˜60% Ethyl Acetate in petroleum ether) to give the title product (3R)-4-[7-(2-methanesulfonylphenyl)-3-[3-methyl-1-(oxan-2-yl)-1H-pyrazol-5-yl]-[1,2]thiazolo[4,5-b]pyridin-5-yl]-3-methylmorpholine (37 mg, 0.067 mmol, 85.53%). LC-MS(ESI+): m/z (M+H)=553.8

### Step 5. (3R)-4-[7-(2-methanesulfonylphenyl)-3-(3-methyl-1H-pyrazol-5-yl)-[1,2]thiazolo[4,5-b]pyridin-5-yl]-3-methylmorpholine

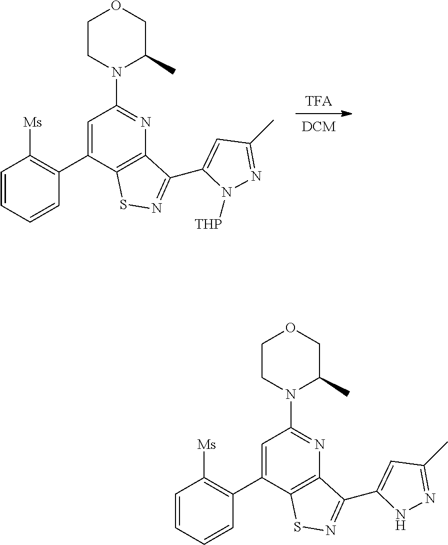

To a solution of (3R)-4-[7-(2-methanesulfonylphenyl)-3-[3-methyl-1-(oxan-2-yl)-1H-pyrazol-5-yl]-[1,2]thiazolo[4,5-b]pyridin-5-yl]-3-methylmorpholine (37 mg, 0.067 mmol) in DCM (2 mL) was added TFA (2 mL) and the resulting mixture was stirred for 3 hrs at ambient temperature. The mixture was concentrated and basified with saturated ammonium. The mixture was concentrated and the residue was purified on flash column chromatography (Silica, 0˜10% MeOH in DCM) and Prep-HPLC (C18, 10-95%, acetonitrile in water with 0.1% formic acid) to give the title product (3R)-4-[7-(2-methanesulfonylphenyl)-3-(3-methyl-1H-pyrazol-5-yl)-[1,2]thiazolo[4,5-b]pyridin-5-yl]-3-methylmorpholine (16.6 mg, 0.035 mmol, 52.90%). LC-MS(ESI+): m/z (M+H)=469.8. ¹H NMR (400 MHz, DMSO) δ 13.09 (br, 1H), 8.20 (d, J=7.7 Hz, 1H), 7.88 (dt, J=15.3, 7.3 Hz, 2H), 7.66 (d, J=7.6 Hz, 1H), 7.35 (s, 1H), 7.16 (s, 1H), 4.47 (d, J=6.2 Hz, 1H), 4.17 (d, J=13.4 Hz, 1H), 4.04 (d, J=8.6 Hz, 1H), 3.79 (d, J=11.3 Hz, 1H), 3.72 (d, J=9.4 Hz, 1H), 3.58 (t, J=10.6 Hz, 1H), 3.26 (t, J=11.0 Hz, 1H), 3.11 (s, 3H), 2.33 (s, 3H), 1.25 (d, J=6.5 Hz, 3H).

### Example 96

### Synthesis of (3R)-3-methyl-4-[3-(3-methyl-1H-pyrazol-5-yl)-7-[2-(trifluoromethyl)pyridin-3-yl]-[1,2]thiazolo[4,5-b]pyridin-5-yl]morpholine

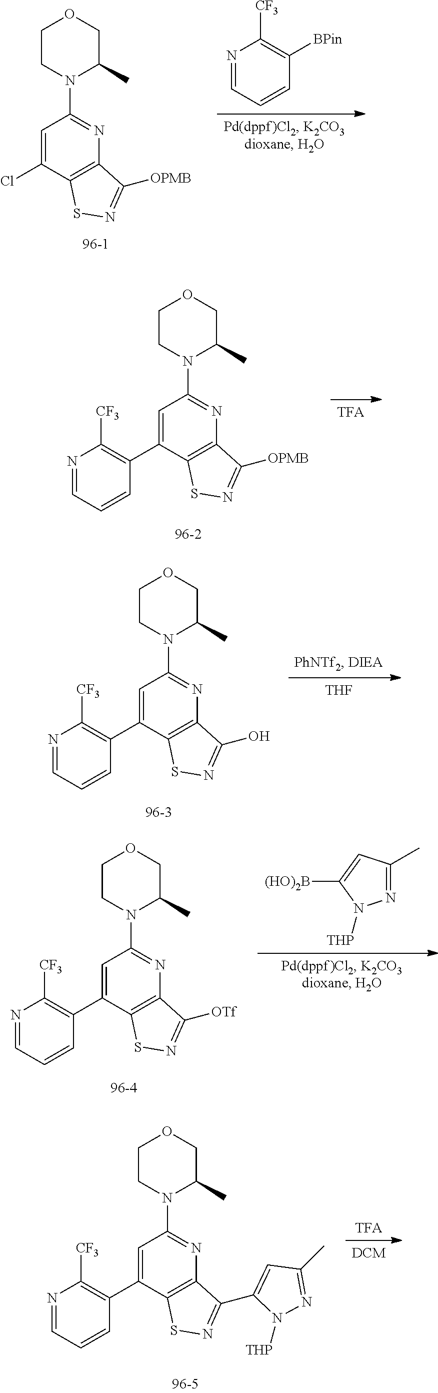

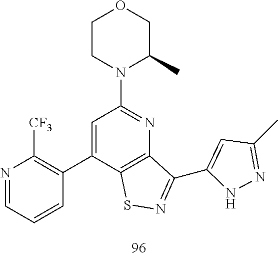 [SMILES: Cc1cc(-c2nsc3c(-c4cccnc4C(F)(F)F)cc(N4CCOC[C@H]4C)nc23)[nH]n1]

### Step 1. (3R)-4-{3-[(4-methoxyphenyl)methoxy]-7-[2-(trifluoromethyl)phenyl]-[1,2]thiazolo[4,5-b]pyridin-5-yl}-3-methylmorpholine

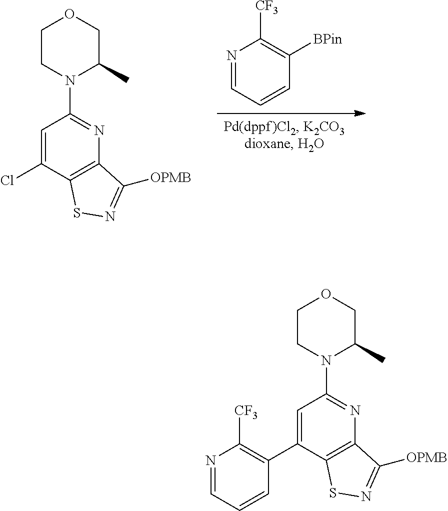

A mixture of (3R)-4-{7-chloro-3-[(4-methoxyphenyl)methoxy]-[1,2]thiazolo[4,5-b]pyridin-5-yl}-3-methylmorpholine (207 mg, 0.510 mmol), 4,4,5,5-tetramethyl-2-[2-(trifluoromethyl)phenyl]-1,3,2-dioxaborolane (277.49 mg, 1.020 mmol), Pd(dppf)Cl₂ (74.63 mg, 0.102 mmol) and K₂CO₃ (110.57 mg, 0.8 mmol) in dioxane (2 mL) and water (0.4 mL) was stirred overnight at 100° C. under nitrogen atmosphere. The reaction mixture was diluted with water and extracted with ethyl acetate. The combined organic layer was washed with brine, dried over Na₂SO₄, filtered and concentrated in vacuo. The residue was purified on flash column chromatography (Silica, 0˜50% Ethyl Acetate in petroleum ether) to give the title product (3R)-4-{3-[(4-methoxyphenyl)methoxy]-7-[2-(trifluoromethyl)phenyl]-[1,2]thiazolo[4,5-b]pyridin-5-yl}-3-methylmorpholine (202 mg, 0.392 mmol, 76.83%). LC-MS(ESI+): m/z (M+H)=516.8

### Step 2. 5-[(3R)-3-methylmorpholin-4-yl]-7-[2-(trifluoromethyl)pyridin-3-yl]-thiazolo[4,5-b]pyridin-3-ol

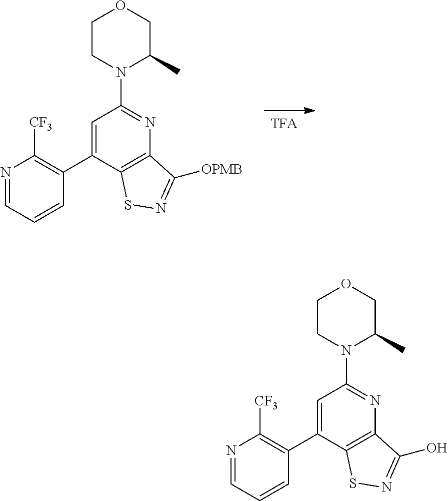

A solution of (3R)-4-{3-[(4-methoxyphenyl)methoxy]-7-[2-(trifluoromethyl)pyridin-3-yl]-[1,2]thiazolo[4,5-b]pyridin-5-yl}-3-methylmorpholine (202 mg, 0.391 mmol) in TFA (5 mL) was stirred at 70° C. for 1 h under nitrogen atmosphere. The reaction mixture was concentrated in vacuo to give the crude title product 5-[(3R)-3-methylmorpholin-4-yl]-7-[2-(trifluoromethyl)pyridin-3-yl]-[1,2]thiazolo[4,5-b]pyridin-3-ol (144 mg, 0.363 mmol, 92.90%). LC-MS(ESI+): m/z (M+H)=396.8

### Step 3. 5-[(3R)-3-methylmorpholin-4-yl]-7-[2-(trifluoromethyl)pyridin-3-yl]-[1,2]thiazolo[4,5-b]pyridin-3-yl trifluoromethanesulfonate

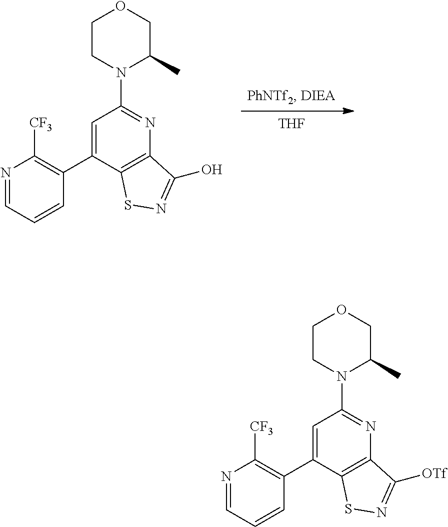 [SMILES: C[C@@H]1COCCN1c1cc(-c2cccnc2C(F)(F)F)c2snc(O)c2n1 ; "PhNTf2, DIEA" ; "THF" ; C[C@@H]1COCCN1c1cc(-c2cccnc2C(F)(F)F)c2snc(OS(=O)(=O)C(F)(F)F)c2n1]

A mixture of 5-[(3R)-3-methylmorpholin-4-yl]-7-[2-(trifluoromethyl)pyridin-3-yl]-[1,2]thiazolo[4,5-b]pyridin-3-ol (144 mg, 0.363 mmol), 1,1,1-trifluoro-N-phenyl-N-trifluoromethanesulfonylmethanesulfonamide (389.34 mg, 1.090 mmol) and DIEA (0.600 mL, 3. mmol) in THF (10 mL) was stirred at 70° C. for 2 hrs under nitrogen atmosphere. After diluted with water, the reaction mixture was extracted with ethyl acetate and the organic layer was washed with brine, dried over Na₂SO₄, filtered and concentrated. The residue was purified on flash column chromatography (Silica, 0˜60% Ethyl Acetate in petroleum ether) to give the title product 5-[(3R)-3-methylmorpholin-4-yl]-7-[2-(trifluoromethyl)pyridin-3-yl]-[1,2]thiazolo[4,5-b]pyridin-3-yl trifluoromethanesulfonate (108 mg, 0.204 mmol, 56.26%). LC-MS(ESI+): m/z (M+H)=528.7.

### Step 4. (3R)-3-methyl-4-{3-[3-methyl-1-(oxan-2-yl)-1H-pyrazol-5-yl]-7-[2-(trifluoromethyl)pyridin-3-yl]-[1,2]thiazolo[4,5-b]pyridin-5-yl}morpholine

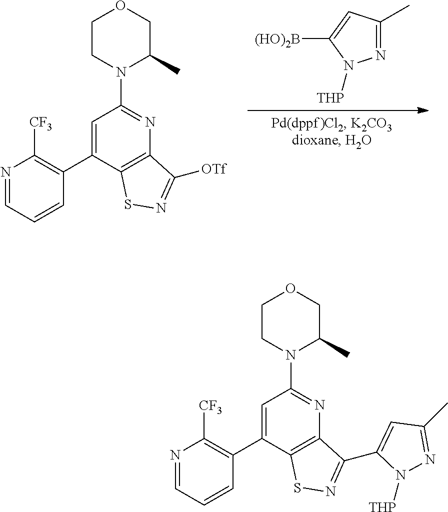

A mixture of 5-[(3R)-3-methylmorpholin-4-yl]-7-[2-(trifluoromethyl)pyridin-3-yl]-[1,2]thiazolo[4,5-b]pyridin-3-yl trifluoromethanesulfonate (54 mg, 0.102 mmol), [3-methyl-1-(oxan-2-yl)-1H-pyrazol-5-yl]boronic acid (85.85 mg, 0.409 mmol), Pd(dppf)Cl₂ (14.95 mg, 0.020 mmol) and K₂CO₃ (82.93 mg, 0.6 mmol) in dioxane (2 mL) and water (0.3 mL) was stirred overnight at 100° C. under nitrogen atmosphere. The reaction mixture was diluted with water and extracted with ethyl acetate. The combined organic layer was washed with brine, dried over Na₂SO₄, filtered and concentrated in vacuo. The residue was purified on flash column chromatography (Silica, 0˜60% Ethyl Acetate in petroleum ether) to give the title product (3R)-3-methyl-4-{3-[3-methyl-1-(oxan-2-yl)-1H-pyrazol-5-yl]-7-[2-(trifluoromethyl)pyridin-3-yl]-[1,2]thiazolo[4,5-b]pyridin-5-yl}morpholine (37 mg, 0.068 mmol, 66.49%). LC-MS(ESI+): m/z (M+H)=544.9 Step 5. (3R)-3-methyl-4-[3-(3-methyl-1H-pyrazol-5-yl)-7-[2-(trifluoromethyl)pyridin-3-yl]-[1,2]thiazolo[4,5-b]pyridin-5-yl]morpholine

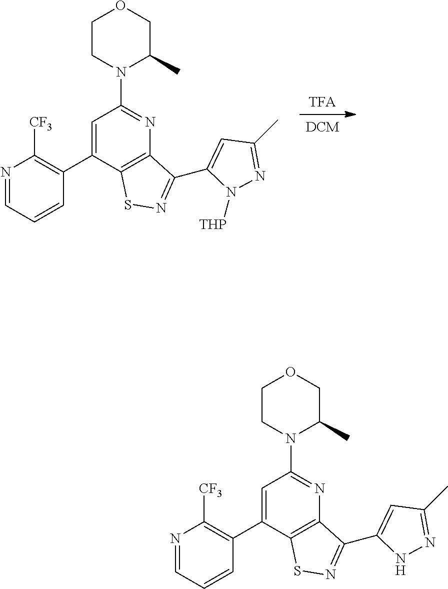

To a solution of (3R)-3-methyl-4-{3-[3-methyl-1-(oxan-2-yl)-1H-pyrazol-5-yl]-7-[2-(trifluoromethyl)pyridin-3-yl]-[1,2]thiazolo[4,5-b]pyridin-5-yl}morpholine (37 mg, 0.068 mmol) in DCM (2 mL) was added TFA (2 mL) and the resulting mixture was stirred for 1 h at ambient temperature. The mixture was concentrated and basified with saturated ammonium. The mixture was concentrated and the residue was purified on flash column chromatography (Silica, 0˜10% MeOH in DCM) and Prep-HPLC (C18, 10-95%, acetonitrile in water with 0.1% formic acid) to give the title product (3R)-3-methyl-4-[3-(3-methyl-1H-pyrazol-5-yl)-7-[2-(trifluoromethyl)pyridin-3-yl]-[1,2]thiazolo[4,5-b]pyridin-5-yl]morpholine (10.2 mg, 0.022 mmol, 32.60%). LC-MS(ESI+): m/z (M+H)=460.8. ¹H NMR (400 MHz, DMSO) δ 13.53-12.65 (m, 1H), 8.95 (d, J=4.3 Hz, 1H), 8.23 (d, J=7.7 Hz, 1H), 7.94 (dd, J=7.8, 4.8 Hz, 1H), 7.32 (s, 1H), 7.16 (s, 1H), 4.47 (d, J=6.2 Hz, 1H), 4.16 (d, J=12.9 Hz, 1H), 4.04 (d, J=8.5 Hz, 1H), 3.80 (d, J=11.3 Hz, 1H), 3.72 (d, J=9.0 Hz, 1H), 3.58 (t, J=10.6 Hz, 1H), 3.25 (d, J=12.6 Hz, 1H), 2.33 (s, 3H), 1.24 (d, J=6.5 Hz, 3H).

### Example 97

### Synthesis of (R)-2-methyl-2-(3-(3-methyl-1H-pyrazol-5-yl)-5-(3-methylmorpholino)isothiazolo[4,5-b]pyridin-7-yl)propan-1-ol

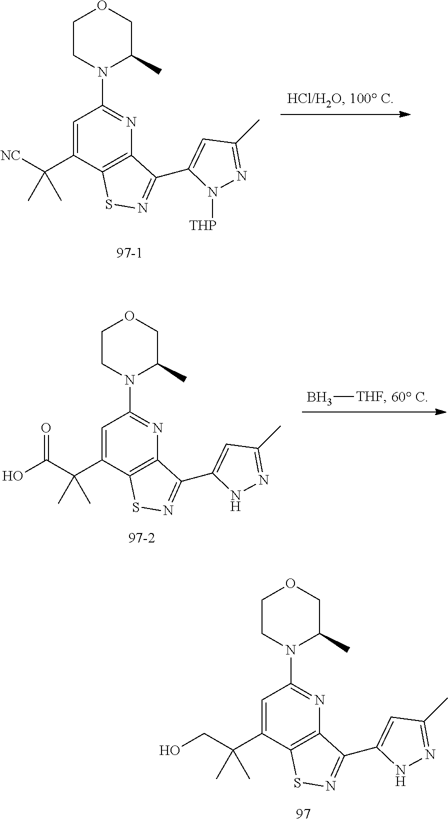

### Step 1. (R)-2-methyl-2-(3-(3-methyl-1H-pyrazol-5-yl)-5-(3-methylmorpholino)isothiazolo[4,5-b]pyridin-7-yl)propanoic acid

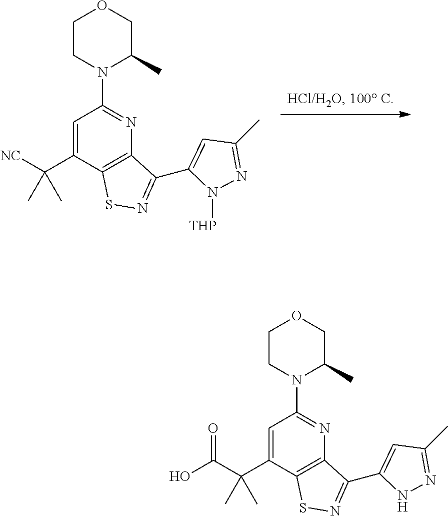

A mixture of 2-methyl-2-{3-[3-methyl-1-(oxan-2-yl)-1H-pyrazol-5-yl]-5-[(3R)-3-methylmorpholin-4-yl]-[1,2]thiazolo[4,5-b]pyridin-7-yl}propanenitrile (130 mg, 0.27 mmol) in HCl/H₂O (10 mL) was stirred at 100° C. for 16 h. LCMS showed the reaction was completed. After concentration, the residue was used for the next step without further purification. LC/MS (ESI): m/z 402 [M+H]⁺.

### Step 2. (R)-2-methyl-2-(3-(3-methyl-1H-pyrazol-5-yl)-5-(3-methylmorpholino)isothiazolo[4,5-b]pyridin-7-yl)propan-1-ol

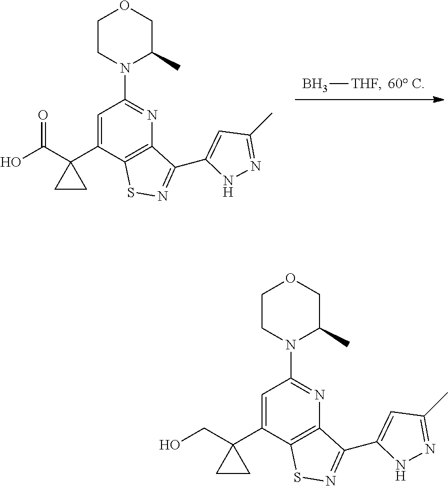

To a solution of 2-methyl-2-[3-(3-methyl-1H-pyrazol-5-yl)-5-[(3R)-3-methylmorpholin-4-yl]-[1,2]thiazolo[4,5-b]pyridin-7-yl]propanoic acid (100 mg, 0.24 mmol) in anhydrous THF (5 mL) was added BH₃ in THF (2.0 M, 0.6 mL, 1.24 mmol) slowly. The resulting mixture was stirred at 60° C. for 1 h. LCMS showed the reaction was completed. The reaction mixture was quenched with HCl/H₂ (1.0 M) and extracted with EA. The organic layer was dried over Na₂SO₄, filtered and concentrated in vacuo. The residue was purified by prep-HPLC (C18, 10-95%, MeOH in H₂O with 0.1% HCOOH) to give the desired product (20 mg, yield: 20%). LC/MS (ESI): m/z 388 [M+H]⁺. 1H NMR (400 MHz, DMSO) δ 7.11 (s, 1H), 7.00 (s, 1H), 4.90 (s, 1H), 4.51 (d, J=5.1 Hz, 1H), 4.06 (t, J=13.7 Hz, 2H), 3.82 (d, J=11.4 Hz, 1H), 3.75-3.64 (m, 3H), 3.57 (t, J=10.5 Hz, 1H), 3.23 (d, J=12.2 Hz, 1H), 2.30 (s, 3H), 1.41 (s, 6H), 1.23 (d, J=6.6 Hz, 3H).

### Example 98

### Synthesis of (R)-(1-(3-(3-methyl-1H-pyrazol-5-yl)-5-(3-methylmorpholino)isothiazolo[4,5-b]pyridin-7-yl)cyclopropyl)methanol

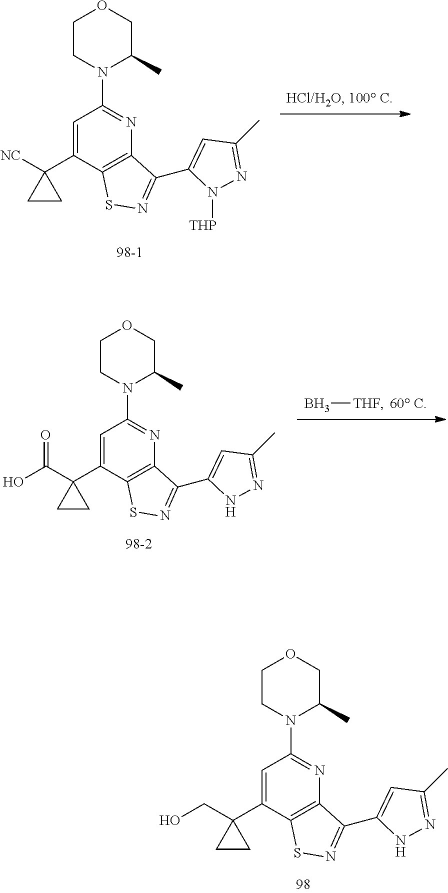

### Step 1. (R)-1-(3-(3-methyl-1H-pyrazol-5-yl)-5-(3-methylmorpholino)isothiazolo[4,5-b]pyridin-7-yl)cyclopropane-1-carboxylic acid

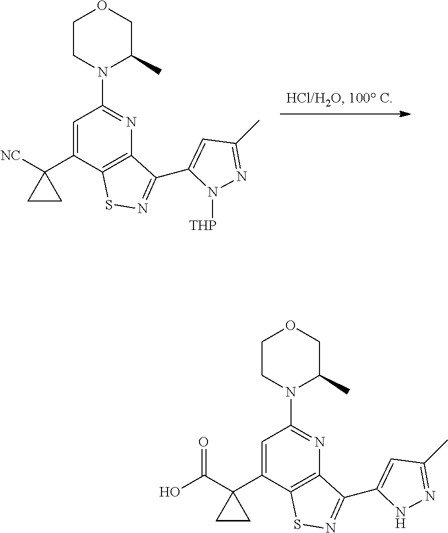

A mixture of 1-(3-(3-methyl-1-(tetrahydro-2H-pyran-2-yl)-1H-pyrazol-5-yl)-5-((R)-3-methylmorpholino)isothiazolo[4,5-b]pyridin-7-yl)cyclopropane-1-carbonitrile (70 mg, 0.15 mmol) in HCl/H₂ (10 mL) was stirred at 100° C. for 16 h. LCMS showed the reaction was completed. After concentration, the residue was used for the next step without further purification. LC/MS (ESI): m/z 400 [M+H]⁺.

### Step 2. (R)-(1-(3-(3-methyl-1H-pyrazol-5-yl)-5-(3-methylmorpholino)isothiazolo[4,5-b]pyridin-7-yl)cyclopropyl)methanol

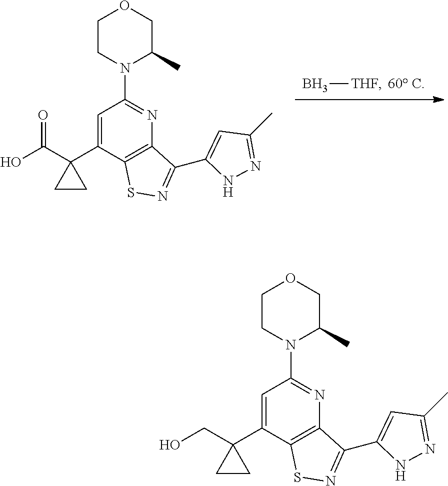

To a solution of 1-[3-(3-methyl-1H-pyrazol-5-yl)-5-[(3R)-3-methylmorpholin-4-yl]-[1,2]thiazolo[4,5-b]pyridin-7-yl]cyclopropane-1-carboxylic acid (50 mg, 0.12 mmol) in anhydrous THF (3 mL) was added BH₃ in THF (2.0 M, 0.3 mL, 0.62 mmol) slowly. The resulting mixture was stirred at 60° C. for 1 h. LCMS showed the reaction was completed. The reaction mixture was quenched with HCl/H₂ (1.0 M) and extracted with EA. The organic layer was dried over Na₂SO₄, filtered and concentrated in vacuo. The residue was purified by prep-HPLC (C18, 10-95%, MeOH in H₂O with 0.1% HCOOH) to give the desired product (5 mg, yield: 10%). LC/MS (ESI): m/z 386 [M+H]⁺. 1H NMR (400 MHz, DMSO-d6) δ 13.02 (d, J=115.0 Hz, 1H), 7.11 (s, 2H), 4.90 (s, 1H), 4.49 (s, 1H), 4.05 (dd, J=24.6, 11.1 Hz, 2H), 3.80 (d, J=11.3 Hz, 1H), 3.70 (d, J=9.6 Hz, 1H), 3.62-3.50 (m, 3H), 3.22 (t, J=11.0 Hz, 1H), 2.30 (s, 3H), 1.22 (d, J=6.6 Hz, 3H), 0.95 (s, 4H).

### Example 99

### Synthesis of (R)-3-methyl-4-(7-(1-methyl-1H-1,2,3-triazol-5-yl)-3-(3-methyl-1H-pyrazol-5-yl)isothiazolo[4,5-b]pyridin-5-yl)morpholine

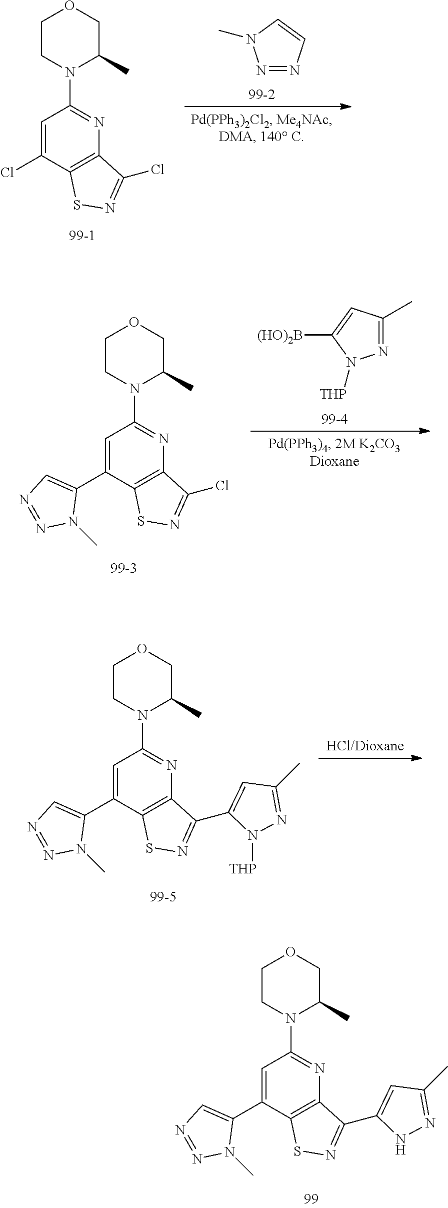

### Step 1. (R)-4-(3-chloro-7-(1-methyl-1H-1,2,3-triazol-5-yl)isothiazolo[4,5-b]pyridin-5-yl)-3-methylmorpholine

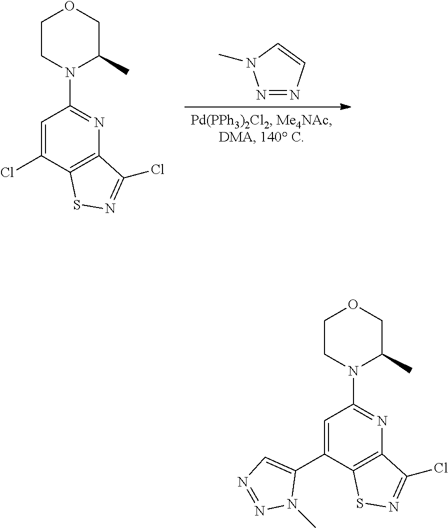

To a mixture of (3R)-4-{3,7-dichloro-[1,2]thiazolo[4,5-b]pyridin-5-yl}-3-methylmorpholine (250 mg, 0.822 mmol), 1-methyl-1H-1,2,3-triazole (410 mg, 4.93 mmol) and Me₄NAc (289 mg, 2.46 mmol) in DMA (10 mL) was added Pd(PPh₃)₂Cl₂ (115 mg, 0.164 mmol). After the mixture was stirred at 140° C. for 12 h under N₂.

LCMS showed the reaction was complete. The mixture was poured into H₂O and extracted with EA (30 mL*3). The combined organic phase was washed brine, dried over Na₂SO₄, filtered and concentrated to dryness. The residue was purified by flash chromatography (silica gel (12 g), 0-100%, EA in PE) to give (R)-4-(3-chloro-7-(1-methyl-1H-1,2,3-triazol-5-yl)isothiazolo[4,5-b]pyridin-5-yl)-3-methylmorpholine (200 mg, 0.570 mmol, 69%). LC/MS (ESI): m/z 351.8/352.5 [M+1]⁺.

### Step 2. (3R)-3-methyl-4-(3-(3-methyl-1-(tetrahydro-2H-pyran-2-yl)-1H-pyrazol-5-yl)-7-(1-methyl-1H-1,2,3-triazol-5-yl)isothiazolo[4,5-b]pyridin-5-yl)morpholine

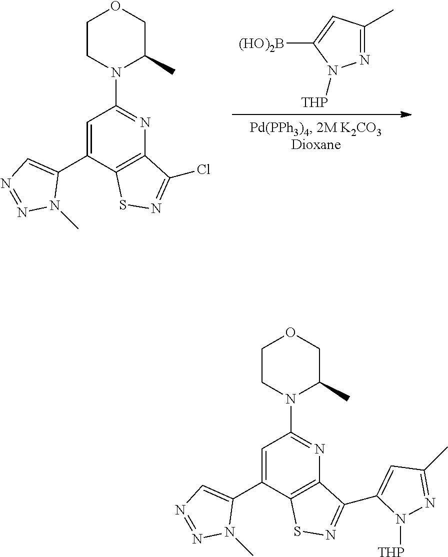

To a mixture of (R)-4-(3-chloro-7-(1-methyl-1H-1,2,3-triazol-5-yl)isothiazolo[4,5-b]pyridin-5-yl)-3-methylmorpholine (100 mg, 0.285 mmol), [3-methyl-1-(oxan-2-yl)-1H-pyrazol-5-yl]boronic acid (180 mg, 0.855 mmol) and K₂CO₃ (0.713 mL, 1.42 mmol, 2M in H₂O) in dioxane (8 mL) was added tetrakis(triphenylphosphane) palladium (66 mg, 0.057 mmol). The mixture was stirred at 100° C. for 16 h under N₂. LCMS showed the reaction was complete. The mixture was filtered and concentrated to dryness. The residue was purified by flash chromatography (silica gel (12 g), 0-100%, EA in PE) to give (3R)-3-methyl-4-(3-(3-methyl-1-(tetrahydro-2H-pyran-2-yl)-1H-pyrazol-5-yl)-7-(1-methyl-1H-1,2,3-triazol-5-yl)isothiazolo[4,5-b]pyridin-5-yl)morpholine (60 mg, 0.125 mmol, 44%). LC/MS (ESI): m/z 481.7 [M+1]⁺.

### Step 3. (R)-3-methyl-4-(7-(1-methyl-1H-1,2,3-triazol-5-yl)-3-(3-methyl-1H-pyrazol-5-yl)isothiazolo[4,5-b]pyridin-5-yl)morpholine

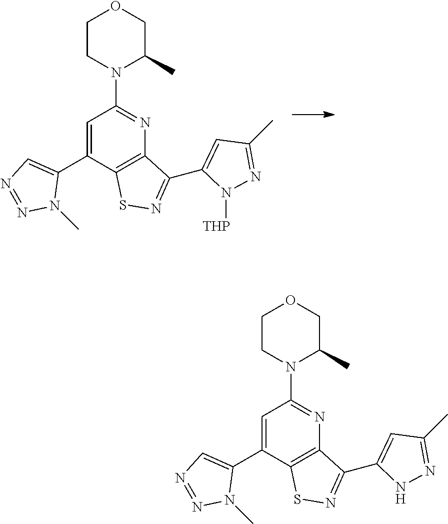

To a mixture of (3R)-3-methyl-4-(3-(3-methyl-1-(tetrahydro-2H-pyran-2-yl)-1H-pyrazol-5-yl)-7-(1-methyl-1H-1,2,3-triazol-5-yl)isothiazolo[4,5-b]pyridin-5-yl)morpholine (60 mg, 0.125 mmol) in DCM (0.5 mL) was added HCl/dioxane (1.5 mL, 4M). After the mixture was stirred at rt for 1 hr. LCMS showed the reaction was complete. The mixture was concentrated to dryness. Then the crude product was purified by Prep-HPLC (C₁₈, 10-95%, MeCN in H₂O with 0.1% HCOOH) to give (R)-3-methyl-4-(7-(1-methyl-1H-1,2,3-triazol-5-yl)-3-(3-methyl-1H-pyrazol-5-yl)isothiazolo[4,5-b]pyridin-5-yl)morpholine (18 mg, 0.045 mmol, 36%). LC/MS (ESI): m/z 397.5 [M+H]⁺. ¹H NMR (400 MHz, DMSO-d6) δ 13.12 (d, J=127.1 Hz, 1H), 8.25 (s, 1H), 7.50 (s, 1H), 7.16 (s, 1H), 4.61-4.53 (m, 1H), 4.21 (s, 3H), 4.20-4.14 (m, 1H), 4.06 (d, J=10.3 Hz, 1H), 3.83 (d, J=11.3 Hz, 1H), 3.77-3.71 (m, 1H), 3.63-3.54 (m, 1H), 3.31-3.23 (m, 1H), 2.32 (s, 3H), 1.27 (d, J=6.6 Hz, 3H).

### Example 100

### Synthesis of (R)-2-methyl-2-(5-(3-methylmorpholino)-3-(1H-pyrazol-5-yl)isothiazolo[4,5-b]pyridin-7-yl)propan-1-ol

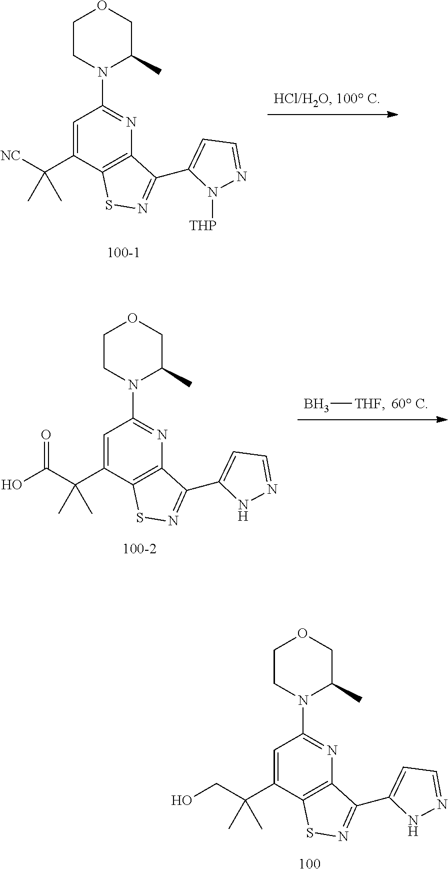

### Step 1. (R)-2-methyl-2-(5-(3-methylmorpholino)-3-(1H-pyrazol-5-yl)isothiazolo[4,5-b]pyridin-7-yl)propanoic acid

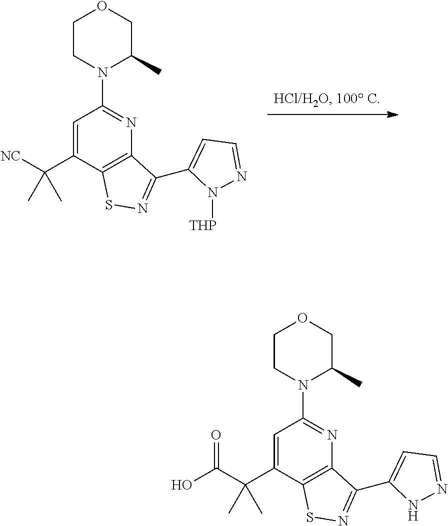

A mixture of 2-methyl-2-{5-[(3R)-3-methylmorpholin-4-yl]-3-[1-(oxan-2-yl)-1H-pyrazol-5-yl]-[1,2]thiazolo[4,5-b]pyridin-7-yl}propanenitrile (150 mg, 0.33 mmol) in HCl/H₂O (20 mL) was stirred at 100° C. for 16 h. LCMS showed the reaction was completed. After concentration, the residue was used for the next step without further purification. LC/MS (ESI): m/z 388 [M+H]⁺.

### Step 2. (R)-2-methyl-2-(5-(3-methylmorpholino)-3-(1H-pyrazol-5-yl)isothiazolo[4,5-b]pyridin-7-yl)propan-1-ol

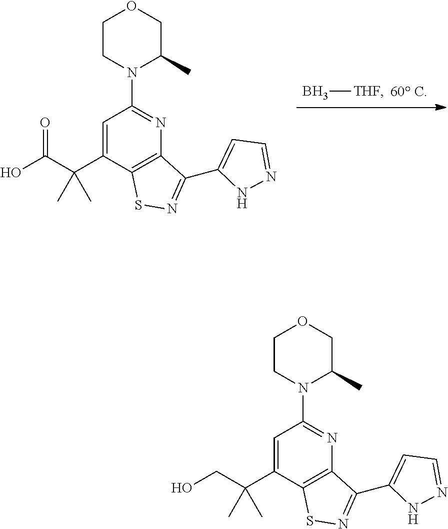

To a solution of 2-methyl-2-{5-[(3R)-3-methylmorpholin-4-yl]-3-(1H-pyrazol-5-yl)-[1,2]thiazolo[4,5-b]pyridin-7-yl}propanoic acid (100 mg, 0.25 mmol) in THF (3 mL) was added BH₃ (2.0 M in THF, 0.6 mL, 1.29 mmol) slowly. The resulting mixture was stirred at 60° C. for 1 h. LCMS showed the reaction was completed. The reaction mixture was quenched with HCl/H₂ (1.0 M) and extracted with EA. The organic layer was dried over Na₂SO₄, filtered and concentrated in vacuo. The residue was purified by Prep-HPLC (C18, 10-95%, MeOH in H₂O with 0.1% HCOOH) to give the desired product (20 mg, yield: 20%). LC/MS (ESI): m/z 374 [M+H]⁺. 1H NMR (400 MHz, DMSO) δ 13.61 (s, 1H), 7.69 (s, 1H), 7.39 (d, J=1.4 Hz, 1H), 7.01 (s, 1H), 4.92 (t, J=5.0 Hz, 1H), 4.53 (d, J=5.8 Hz, 1H), 4.11-3.99 (m, 2H), 3.82 (d, J=11.4 Hz, 1H), 3.69 (dd, J=14.6, 8.5 Hz, 3H), 3.57 (t, J=10.3 Hz, 1H), 3.25 (dd, J=12.4, 9.5 Hz, 1H), 1.41 (s, 6H), 1.23 (d, J=6.6 Hz, 3H).

### Example 101

### Synthesis of (3R)-3-methyl-4-[3-(1H-pyrazol-5-yl)-7-[2-(trifluoromethyl)pyridin-3-yl]-[1,2]thiazolo[4,5-b]pyridin-5-yl]morpholine

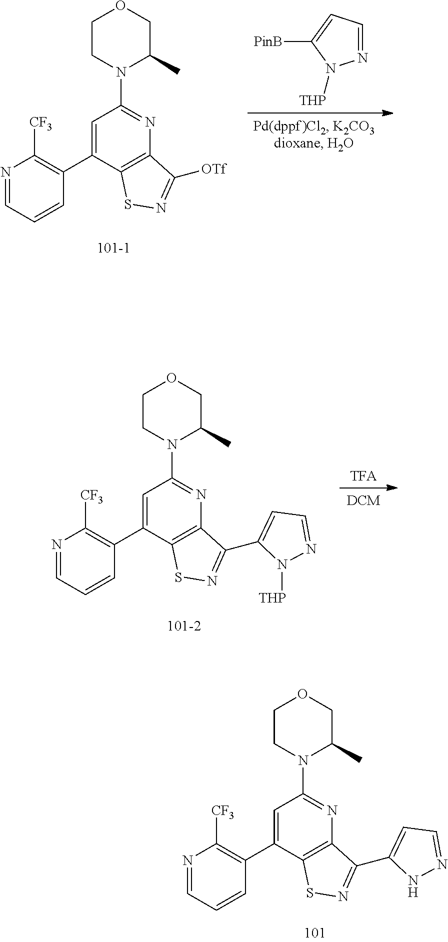

### Step 1. (3R)-3-methyl-4-{3-[1-(oxan-2-yl)-1H-pyrazol-5-yl]-7-[2-(trifluoromethyl)pyridin-3-yl]-[1,2]thiazolo[4,5-b]pyridin-5-yl}morpholine

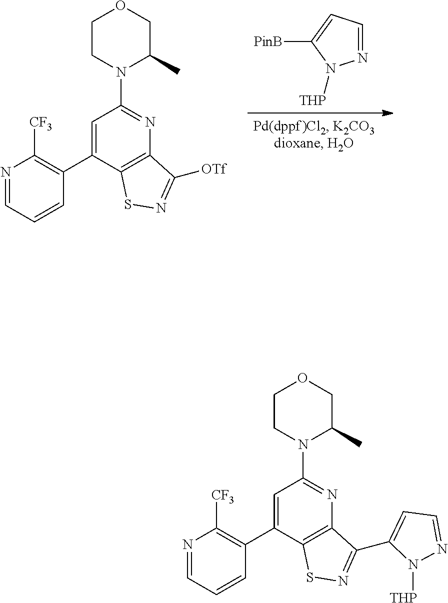

A mixture of 5-[(3R)-3-methylmorpholin-4-yl]-7-[2-(trifluoromethyl)pyridin-3-yl]-[1,2]thiazolo[4,5-b]pyridin-3-yl trifluoromethanesulfonate (54 mg, 0.102 mmol), 1-(oxan-2-yl)-5-(tetramethyl-1,3,2-dioxaborolan-2-yl)-1H-pyrazole (56.74 mg, 0.204 mmol), Pd(dppf)Cl₂ (14.95 mg, 0.020 mmol) and K₂CO₃ (82.93 mg, 0.6 mmol) in dioxane (2 mL) and water (0.3 mL) was stirred overnight at 100° C. under nitrogen atmosphere. The reaction mixture was diluted with water and extracted with ethyl acetate. The combined organic layer was washed with brine, dried over Na₂SO₄, filtered and concentrated in vacuo. The residue was purified on flash column chromatography (Silica, 0˜60% Ethyl Acetate in petroleum ether) to give the title product (3R)-3-methyl-4-{3-[1-(oxan-2-yl)-1H-pyrazol-5-yl]-7-[2-(trifluoromethyl)pyridin-3-yl]-[1,2]thiazolo[4,5-b]pyridin-5-yl}morpholine (27 mg, 0.051 mmol, 49.89%). LC-MS(ESI+): m/z (M+H)=530.8 Step 2. (3R)-3-methyl-4-[3-(1H-pyrazol-5-yl)-7-[2-(trifluoromethyl)pyridin-3-yl]-[1,2]thiazolo[4,5-b]pyridin-5-yl]morpholine

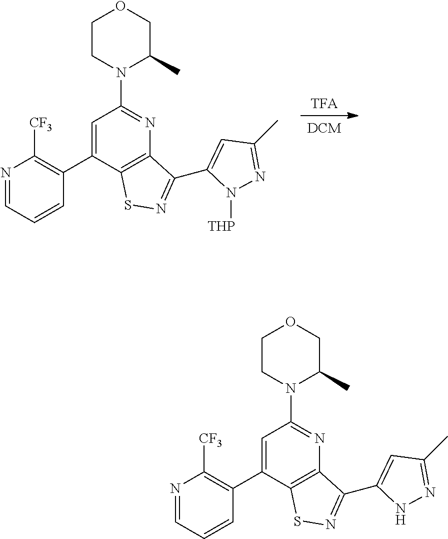

To a solution of (3R)-3-methyl-4-{3-[1-(oxan-2-yl)-1H-pyrazol-5-yl]-7-[2-(trifluoromethyl)pyridin-3-yl]-[1,2]thiazolo[4,5-b]pyridin-5-yl}morpholine (27 mg, 0.051 mmol) in DCM (2 mL) was added TFA (2 mL) and the resulting mixture was stirred for 1 h at ambient temperature. The mixture was concentrated and basified with saturated ammonium. The mixture was concentrated and the residue was purified on flash column chromatography (Silica, 0˜10% MeOH in DCM) and Prep-HPLC (C18, 10-95%, acetonitrile in water with 0.1% formic acid) to give the title product (3R)-3-methyl-4-[3-(1H-pyrazol-5-yl)-7-[2-(trifluoromethyl)pyridin-3-yl]-[1,2]thiazolo[4,5-b]pyridin-5-yl]morpholine (8.2 mg, 0.018 mmol, 36.09%). LC-MS(ESI+): m/z (M+H)=446.8. ¹H NMR (400 MHz, DMSO) δ 14.09-12.88 (m, 1H), 8.95 (d, J=4.5 Hz, 1H), 8.23 (d, J=7.8 Hz, 1H), 7.94 (dd, J=7.8, 4.8 Hz, 1H), 7.79 (br, 1H), 7.44 (d, J=1.4 Hz, 1H), 7.33 (s, 1H), 4.49 (d, J=5.8 Hz, 1H), 4.16 (d, J=13.1 Hz, 1H), 4.04 (d, J=9.0 Hz, 1H), 3.80 (d, J=11.3 Hz, 1H), 3.71 (d, J=9.1 Hz, 1H), 3.58 (t, J=10.4 Hz, 1H), 3.25 (d, J=12.7 Hz, 1H), 1.24 (d, J=6.5 Hz, 3H).

### Example 102

### Synthesis of (R)-3-methyl-4-(7-(1-methyl-1H-1,2,4-triazol-5-yl)-3-(3-methyl-1H-pyrazol-5-yl)isothiazolo[4,5-b]pyridin-5-yl)morpholine

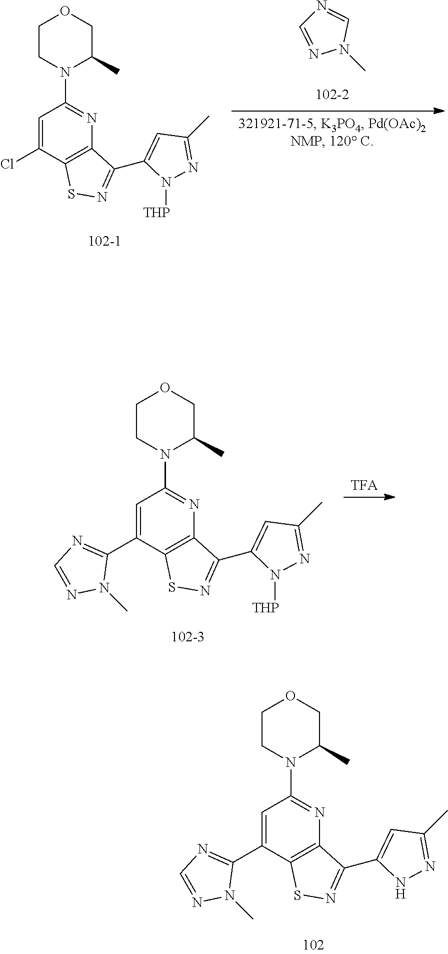

### Step 1. (3R)-3-methyl-4-(3-(3-methyl-1-(tetrahydro-2H-pyran-2-yl)-1H-pyrazol-5-yl)-7-(1-methyl-1H-1,2,4-triazol-5-yl)isothiazolo[4,5-b]pyridin-5-yl)morpholine

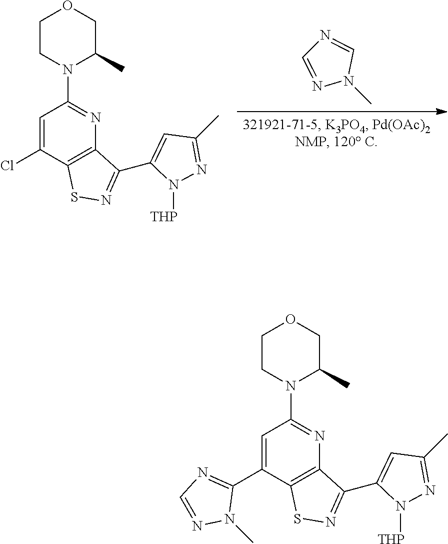

A mixture of (3R)-4-{7-chloro-3-[3-methyl-1-(oxan-2-yl)-1H-pyrazol-5-yl]-[1,2]thiazolo[4,5-b]pyridin-5-yl}-3-methylmorpholine (50 mg, 0.11 mmol), 1-methyl-1H-1,2,4-triazole (19 mg, 0.23 mmol), Butyldi-1-adamantylphosphine (4 mg, 0.01 mmol), K₃PO₄ (48 mg, 0.23 mmol), and Pd(OAc)₂ (2 mg, 0.01 mmol) in NMP (2 mL) was stirred at 120° C. under N₂ atmosphere for 16 h. LCMS showed the reaction was completed. The reaction mixture was diluted with H₂O and extracted with EA. The combined organic layer was dried over Na₂SO₄, filtered and concentrated in vacuum. The residue was purified by column chromatography on silica gel (DCM:MeOH=40:1, V/V) to afford the desired product (20 mg, yield: 36%). LC/MS (ESI): m/z 481 [M+H]⁺.

### Step 2. (R)-3-methyl-4-(7-(1-methyl-1H-1,2,4-triazol-5-yl)-3-(3-methyl-1H-pyrazol-5-yl)isothiazolo[4,5-b]pyridin-5-yl)morpholine

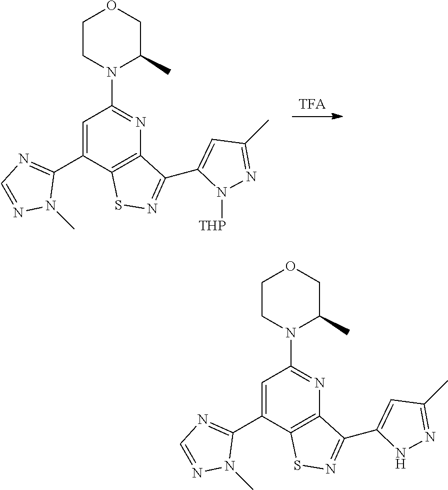

A mixture of (3R)-3-methyl-4-{3-[3-methyl-1-(oxan-2-yl)-1H-pyrazol-5-yl]-7-(1-methyl-1H-1,2,4-triazol-5-yl)-[1,2]thiazolo[4,5-b]pyridin-5-yl}morpholine (26 mg, 0.05 mmol) in TFA (2 mL) was stirred at room temperature for 2 h. After concentration, the residue was purified by prep-HPLC (C18, 10-95%, MeOH in H₂O with 0.1% HCOOH) to give the desired product (1.1 mg, yield: 5%). LC/MS (ESI): m/z 397 [M+H]⁺. 1H NMR (400 MHz, DMSO) δ 13.07 (d, J=117.2 Hz, 1H), 8.29 (s, 1H), 7.59 (s, 1H), 7.13 (s, 1H), 4.59 (s, 1H), 4.30 (s, 3H), 4.19 (d, J=12.2 Hz, 1H), 4.08 (d, J=10.3 Hz, 1H), 3.85 (d, J=11.3 Hz, 1H), 3.77 (d, J=9.5 Hz, 1H), 3.61 (d, J=11.6 Hz, 1H), 3.29-3.24 (m, 1H), 2.31 (s, 3H), 1.29 (d, J=6.6 Hz, 3H).

### Example 103

### Synthesis of (R)-3-methyl-4-(7-(1-methyl-1H-1,2,3-triazol-5-yl)-3-(1H-pyrazol-5-yl)isothiazolo[4,5-b]pyridin-5-yl)morpholine

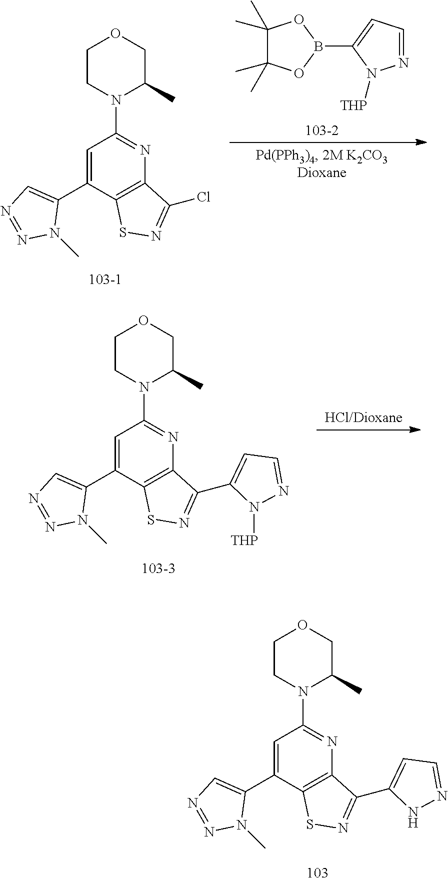

### Step 1. (3R)-3-methyl-4-(7-(1-methyl-1H-1,2,3-triazol-5-yl)-3-(1-(tetrahydro-2H-pyran-2-yl)-1H-pyrazol-5-yl)isothiazolo[4,5-b]pyridin-5-yl)morpholine

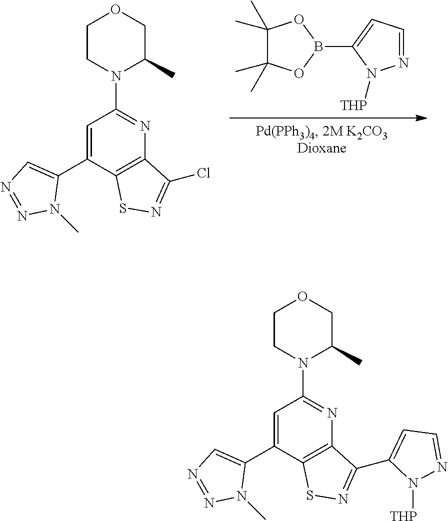

To a mixture of (R)-4-(3-chloro-7-(1-methyl-1H-1,2,3-triazol-5-yl)isothiazolo[4,5-b]pyridin-5-yl)-3-methylmorpholine (95 mg, 0.271 mmol), 1-(oxan-2-yl)-5-(tetramethyl-1,3,2-dioxaborolan-2-yl)-1H-pyrazole (226 mg, 0.812 mmol) and K₂CO₃ (0.677 mL, 1.35 mmol, 2M in H₂O) in dioxane (8 mL) was added Pd(PPh₃)₄ (63 mg, 0.054 mmol). After the mixture was stirred at 100° C. for 16 hs under N₂. LC-MS showed the reaction was complete. The mixture was filtered and concentrated to dryness. The residue was purified by flash chromatography (silica gel (12 g), 0-100%, EA in PE) to give (3R)-3-methyl-4-(7-(1-methyl-1H-1,2,3-triazol-5-yl)-3-(1-(tetrahydro-2H-pyran-2-yl)-1H-pyrazol-5-yl)isothiazolo[4,5-b]pyridin-5-yl)morpholine (52 mg, 0.111 mmol, 41%). LC/MS (ESI): m/z 467.6 [M+1]⁺.

### Step 2. (R)-3-methyl-4-(7-(1-methyl-1H-1,2,3-triazol-5-yl)-3-(1H-pyrazol-5-yl)isothiazolo[4,5-b]pyridin-5-yl)morpholine

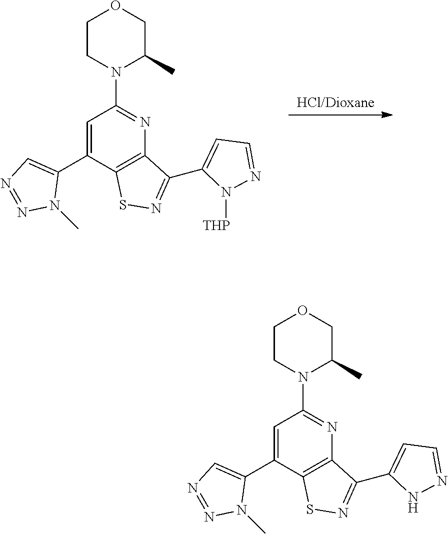

To a mixture of (3R)-3-methyl-4-(7-(1-methyl-1H-1,2,3-triazol-5-yl)-3-(1-(tetrahydro-2H-pyran-2-yl)-1H-pyrazol-5-yl)isothiazolo[4,5-b]pyridin-5-yl)morpholine (52 mg, 0.111 mmol) in DCM (0.5 mL) was added HCl/dioxane (1.5 mL, 4M). After the mixture was stirred at rt for 1 hr. LC-MS showed the reaction was complete. The mixture was concentrated to dryness. Then the crude product was purified by Prep-HPLC (C₁₈, 10-95%, MeCN in H₂O with 0.1% HCOOH) to give (R)-3-methyl-4-(7-(1-methyl-1H-1,2,3-triazol-5-yl)-3-(1H-pyrazol-5-yl)isothiazolo[4,5-b]pyridin-5-yl)morpholine (8 mg, 0.021 mmol, 19%). LC/MS (ESI): m/z 383.5 [M+H]⁺. ¹H NMR (400 MHz, DMSO-d6) δ 13.53 (d, J=193.9 Hz, 1H), 8.26 (s, 1H), 7.77 (s, 1H), 7.49 (s, 1H), 7.43 (s, 1H), 4.61-4.54 (m, 1H), 4.20 (s, 3H), 4.17 (s, 1H), 4.05 (dd, J=10.5, 1.3 Hz, 1H), 3.83 (d, J=11.4 Hz, 1H), 3.76-3.71 (m, 1H), 3.59 (dd, J=12.4, 10.8 Hz, 1H), 3.29-3.25 (m, 1H), 1.27 (d, J=6.6 Hz, 3H).

### Example 104

### Synthesis of (R)-4-(7-chloro-3-(3-methyl-1H-pyrazol-5-yl)isothiazolo[4,5-b]pyridin-5-yl)-3-methylmorpholine

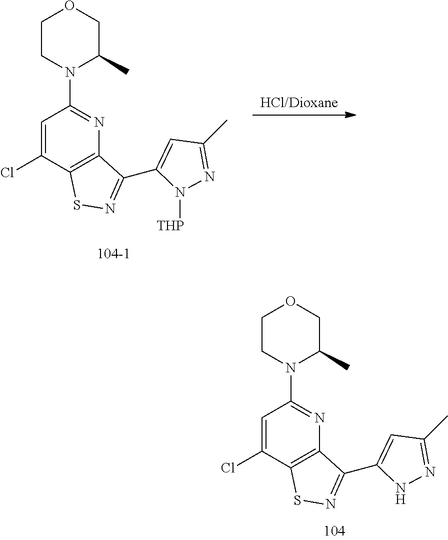

### Step 1. (R)-4-(7-chloro-3-(3-methyl-1H-pyrazol-5-yl)isothiazolo[4,5-b]pyridin-5-yl)-3-methylmorpholine

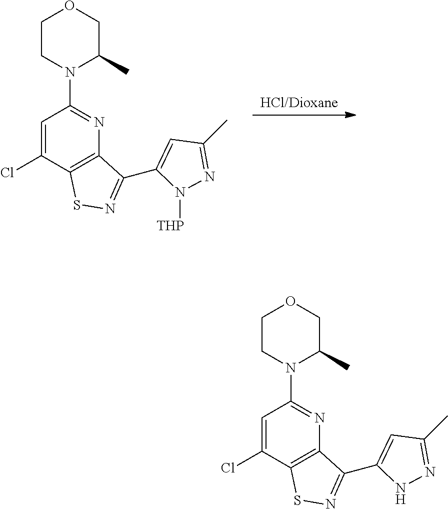

A mixture of (3R)-4-{7-chloro-3-[3-methyl-1-(oxan-2-yl)-1H-pyrazol-5-yl]-[1,2]thiazolo[4,5-b]pyridin-5-yl}-3-methylmorpholine (10 mg, 0.02 mmol) in TFA (2 mL) was stirred at room temperature for 2 h. LC-MS showed the reaction was complete. The reaction mixture was concentrated under reduced pressure. The residue was purified by Prep-HPLC (C18, 10-95%, MeOH in H₂O with 0.1% HCOOH) to give the desired product (2 mg, yield: 24%). LC/MS (ESI): m/z 350 [M+H]⁺. 1H NMR (400 MHz, DMSO) δ 13.13 (d, J=120.2 Hz, 1H), 7.50 (s, 1H), 7.11 (s, 1H), 4.51 (s, 1H), 4.11 (d, J=12.3 Hz, 1H), 4.02 (d, J=11.2 Hz, 1H), 3.80 (d, J=11.5 Hz, 1H), 3.70 (d, J=11.6 Hz, 1H), 3.55 (t, J=11.6 Hz, 1H), 3.24 (d, J=11.9 Hz, 1H), 2.31 (s, 3H), 1.23 (d, J=6.5 Hz, 3H).

### Example 105

### Synthesis of (R)-(4-(3-(3-methyl-1H-pyrazol-5-yl)-5-(3-methylmorpholino)isothiazolo[4,5-b]pyridin-7-yl)tetrahydro-2H-pyran-4-yl)methanol

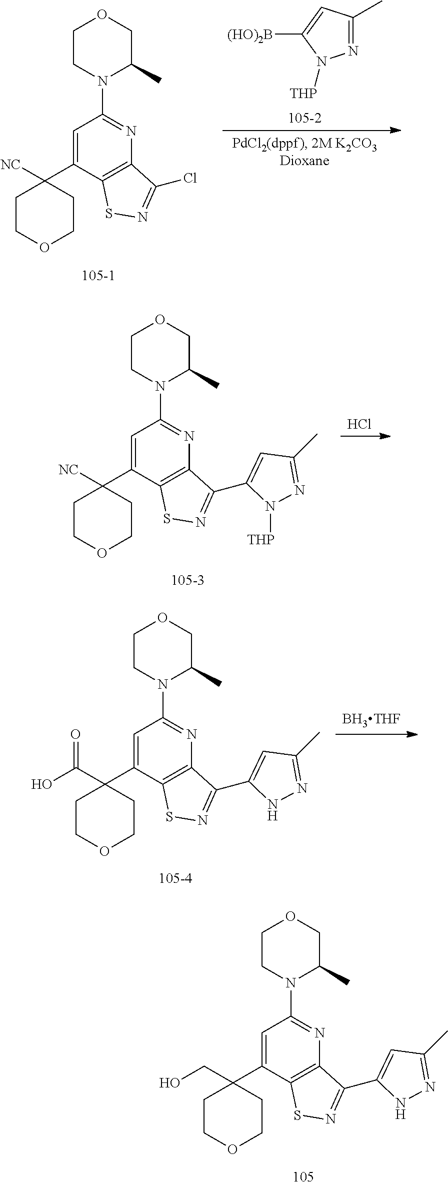

### Step 1. 4-(3-(3-methyl-1-(tetrahydro-2H-pyran-2-yl)-1H-pyrazol-5-yl)-5-((R)-3-methylmorpholino)isothiazolo[4,5-b]pyridin-7-yl)tetrahydro-2H-pyran-4-carbonitrile

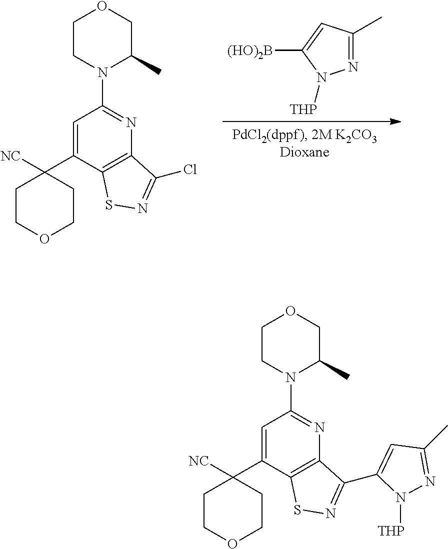

A mixture of 4-{3-chloro-5-[(3R)-3-methylmorpholin-4-yl]-[1,2]thiazolo[4,5-b]pyridin-7-yl}oxane-4-carbonitrile (190 mg, 0.50 mmol), PdCl₂(dppf) (73 mg, 0.10 mmol) and K₂CO₃ (2.0 M in H₂O, 0.7 mL) in dioxane (5 mL) was stirred at 100° C. under N₂ atmosphere for 16 h. LC-MS showed the reaction was complete. The reaction mixture was diluted with EA (40 mL), then washed with water and brine, dried over anhydrous Na₂SO₄, filtered and concentrated. The residue was purified by column chromatography on silica gel (PE:EA=1:2, V/V) to afford the desired product (81 mg, yield: 31%). LC/MS (ESI): m/z 509 [M+H]⁺.

### Step 2. (R)-4-(3-(3-methyl-1H-pyrazol-5-yl)-5-(3-methylmorpholino)isothiazolo[4,5-b]pyridin-7-yl)tetrahydro-2H-pyran-4-carboxylic acid

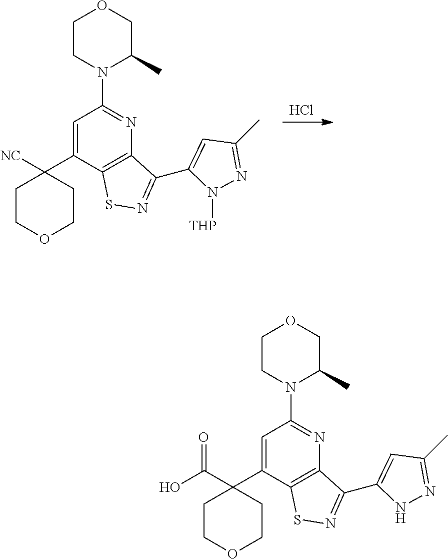

A mixture of 4-{3-[3-methyl-1-(oxan-2-yl)-1H-pyrazol-5-yl]-5-[(3R)-3-methylmorpholin-4-yl]-[1,2]thiazolo[4,5-b]pyridin-7-yl}oxane-4-carbonitrile (81 mg, 0.15 mmol) in HCl/H₂O (10 mL) was stirred at 100° C. for 16 h. LCMS showed the reaction was completed. After concentration, the residue was used for the next step without further purification. LC/MS (ESI): m/z 444 [M+H]⁺.

### Step 3. (R)-(4-(3-(3-methyl-1H-pyrazol-5-yl)-5-(3-methylmorpholino)isothiazolo[4,5-b]pyridin-7-yl)tetrahydro-2H-pyran-4-yl)methanol

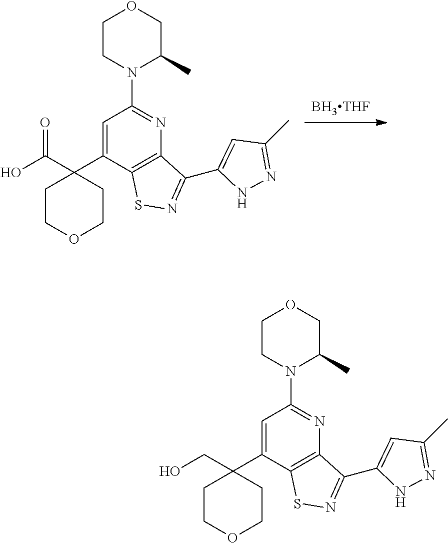 [SMILES: "BH3·THF" ; Cc1cc(-c2nsc3c(C4(C(=O)O)CCOCC4)cc(N4CCOC[C@H]4C)nc23)[nH]n1 ; Cc1cc(-c2nsc3c(C4(CO)CCOCC4)cc(N4CCOC[C@H]4C)nc23)[nH]n1]

To a solution of 4-[3-(3-methyl-1H-pyrazol-5-yl)-5-[(3R)-3-methylmorpholin-4-yl]-[1,2]thiazolo[4,5-b]pyridin-7-yl]oxane-4-carboxylic acid (50 mg, 0.11 mmol) in anhydrous THF (3 mL) was added BH₃-THF (2.0 M, 0.16 mL) at room temperature. The resulting mixture was stirred at 60° C. for 1 h. LCMS showed the reaction was completed. The reaction mixture was quenched with MeOH and concentrated. The residue was diluted with EA and washed with NaHCO₃ aqueous solution. The organic layer was dried over Na₂SO₄, filtered and concentrated in vacuum. The residue was purified by Prep-HPLC (C18, 10-95%, MeOH in H₂O with 0.1% HCOOH) to give the desired product (10 mg, yield: 20%). LC/MS (ESI): m/z 430 [M+H]⁺. 1H NMR (400 MHz, DMSO-d6) δ 13.09 (s, 1H), 7.12 (s, 1H), 7.01 (s, 1H), 4.84 (s, 1H), 4.51 (d, J=6.3 Hz, 1H), 4.07 (dd, J=25.1, 10.3 Hz, 2H), 3.83-3.68 (m, 6H), 3.61-3.47 (m, 3H), 3.25-3.19 (m, 1H), 2.31 (s, 3H), 2.19 (s, 2H), 2.06-1.95 (m, 2H), 1.22 (d, J=6.6 Hz, 3H).

### Example 106

### Synthesis of (R)-1-(3-(3-methyl-1H-pyrazol-5-yl)-5-(3-methylmorpholino)isothiazolo[4,5-b]pyridin-7-yl)cyclopentan-1-ol

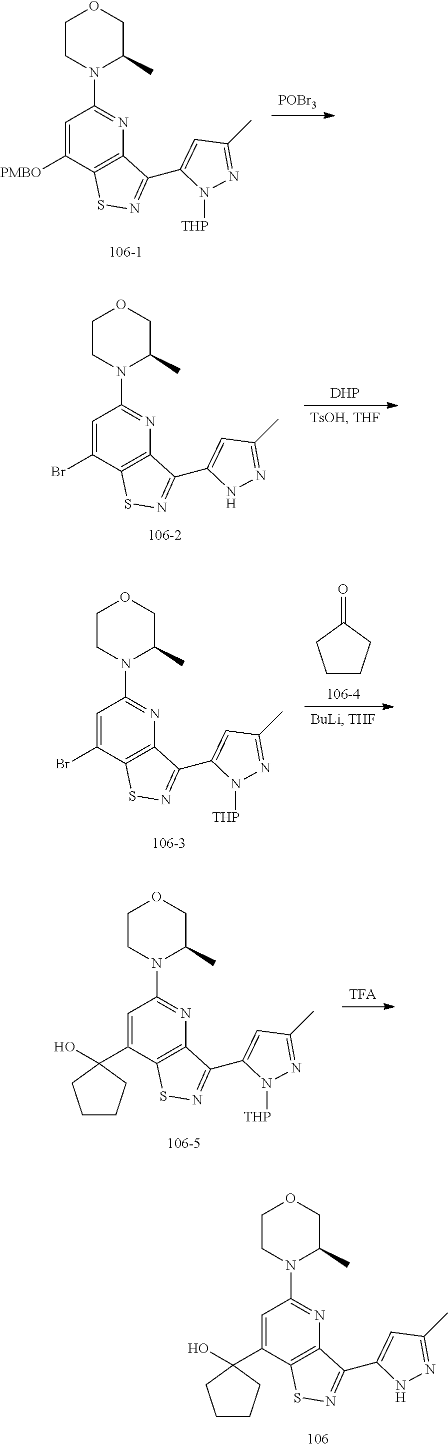

### Step 1. (R)-4-(7-bromo-3-(3-methyl-1H-pyrazol-5-yl)isothiazolo[4,5-b]pyridin-5-yl)-3-methylmorpholine

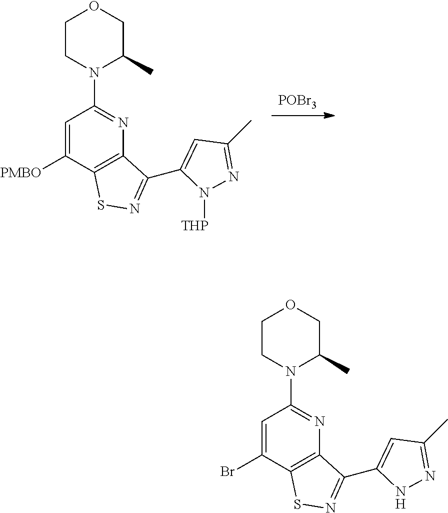

To a solution of (3R)-4-(7-((4-methoxybenzyl)oxy)-3-(3-methyl-1-(tetrahydro-2H-pyran-2-yl)-1H-pyrazol-5-yl)isothiazolo[4,5-b]pyridin-5-yl)-3-methylmorpholine (500 mg, 0.933 mmol) in POBr₃ (0.759 mL, 7.467 mmol). The mixture was stirred at 65° C. for 3 hr. LC-MS showed the reaction was complete. The mixture was diluted with EA (20 mL) and saturated Na₂CO₃ solution (20 mL). The organic layer was separated, washed with further saturated NaCl solution dried over anhydrous Na₂SO₄, filtered and concentrated in vacuo. The residue was purified by column chromatography on silica gel (DCM:MeOH=30:1, V/V) to afford the crude desired product (300 mg, 0.761 mmol, 81.51%). LC/MS (ESI) m/z: 394 (M+H)⁺.

### Step 2. (3R)-4-(7-bromo-3-(3-methyl-1-(tetrahydro-2H-pyran-2-yl)-1H-pyrazol-5-yl)isothiazolo[4,5-b]pyridin-5-yl)-3-methylmorpholine

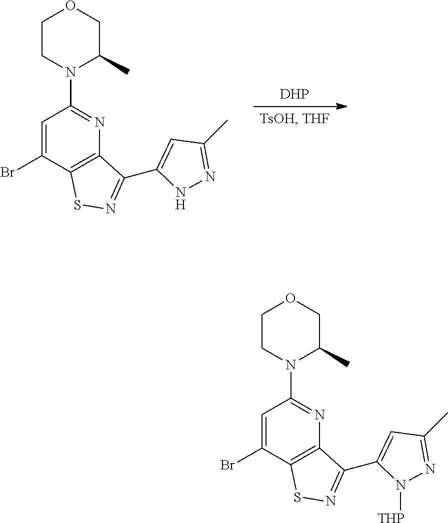

To a solution of (R)-4-(7-bromo-3-(3-methyl-1H-pyrazol-5-yl)isothiazolo[4,5-b]pyridin-5-yl)-3-methylmorpholine (300 mg, 0.789 mmol) in THF (5 mL) were added 3,4-dihydro-2H-pyran (0.278 mL, 3.043 mmol) and p-toluenesulfonic acid (0.024 mL, 0.152 mmol), and the reaction was stirred at 65° C. for 3 hr. The reaction was diluted with DCM and water. The organic layer was separated, washed with further saturated NaCl solution, and concentrated in vacuo. The residue was purified via column chromatography on silica gel (PE:EA=4:1, V/V) to afford the desired product (70 mg, 0.146 mmol, 19.23%). LC/MS (ESI) m/z: 478 (M+H)⁺.

### Step 3. 1-(3-(3-methyl-1-(tetrahydro-2H-pyran-2-yl)-1H-pyrazol-5-yl)-5-((R)-3-methylmorpholino)isothiazolo[4,5-b]pyridin-7-yl)cyclopentan-1-ol

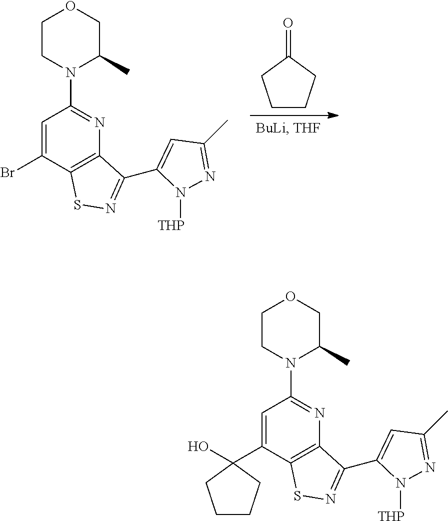

To a solution of (3R)-4-(7-bromo-3-(3-methyl-1-(tetrahydro-2H-pyran-2-yl)-1H-pyrazol-5-yl)isothiazolo[4,5-b]pyridin-5-yl)-3-methylmorpholine (70 mg, 0.146 mmol) and cyclopentanone (0.052 mL, 0.585 mmol) in THF (5 mL) at −70° C. was added n-BuLi (0.234 mL, 0.585 mmol) drop wise. The mixture was stirred at −70° C. for 1 h. LC-MS showed the reaction was complete. The reaction mixture was quenched with saturated NH₄Cl aqueous solution, then extracted with EA (30 mL×3).

The combined organic layer was washed with brine, dried over anhydrous Na₂SO₄, filtered and concentrated. The residue was purified by column chromatography on silica gel (PE:EA=50:1, V/V) to afford the desired product (20 mg, 0.041 mmol, 28.26%). LC/MS (ESI) m/z: 484 [M+H]⁺.

### Step 4. (R)-1-(3-(3-methyl-1H-pyrazol-5-yl)-5-(3-methylmorpholino)isothiazolo[4,5-b]pyridin-7-yl)cyclopentan-1-ol

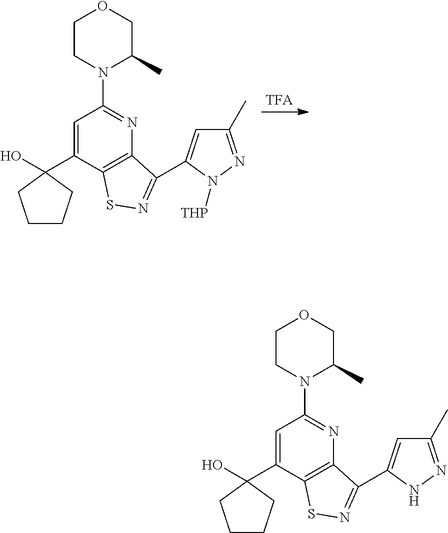

A solution of 1-(3-(3-methyl-1-(tetrahydro-2H-pyran-2-yl)-1H-pyrazol-5-yl)-5-((R)-3-methylmorpholino)isothiazolo[4,5-b]pyridin-7-yl)cyclopentan-1-ol (20 mg, 0.041 mmol) in DCM (1 mL) and TFA (1 mL) was stirred at room temperature for 3 hr. The reaction mixture was concentrated in vacuo. The residue was purified by Pre-HPLC (C₁₈, 10-95%, MeOH in H₂O with 0.1% HCOOH) to afford the desired product (3 mg, 0.008 mmol, 18.32%). LC/MS (ESI) m/z: 399 (M+H)⁺. 1H NMR (400 MHz, DMSO-d6) δ 12.95 (d, J=106.8 Hz, 1H), 7.09 (s, 1H), 7.03 (s, 1H), 5.89 (s, 1H), 4.54 (d, J=4.9 Hz, 1H), 4.09 (d, J=12.3 Hz, 1H), 4.02 (d, J=8.6 Hz, 1H), 3.80 (d, J=11.3 Hz, 1H), 3.71 (d, J=9.4 Hz, 1H), 3.56 (t, J=10.4 Hz, 1H), 3.21 (t, J=11.2 Hz, 1H), 2.29 (s, 3H), 2.04-1.83 (m, 8H), 1.20 (d, J=6.6 Hz, 3H).

### Example 107

### Synthesis of (3R)-4-[7-(1-ethyl-1H-1,2,3-triazol-5-yl)-3-(3-methyl-1H-pyrazol-5-yl)-[1,2]thiazolo[4,5-b]pyridin-5-yl]-3-methylmorpholine

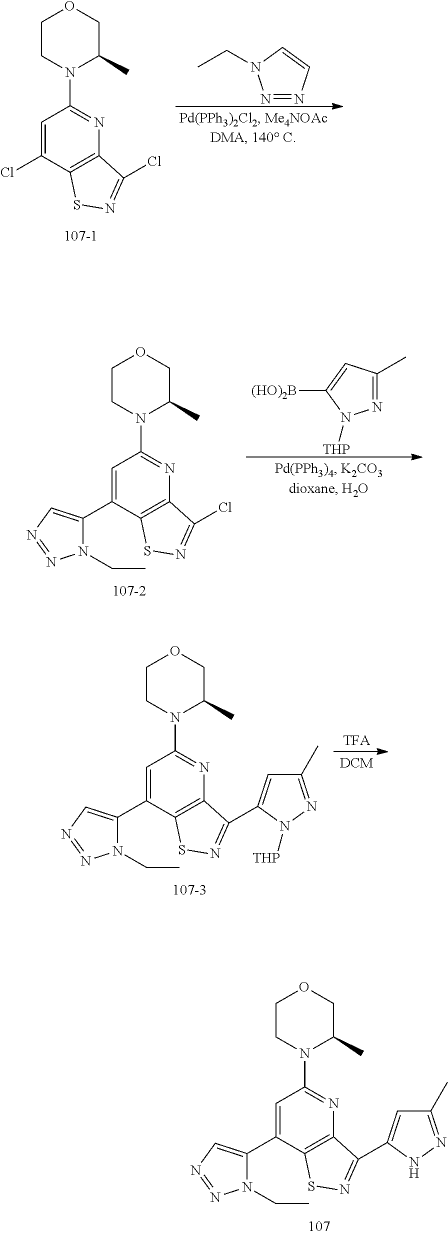

### Step 1. (3R)-4-[3-chloro-7-(1-ethyl-1H-1,2,3-triazol-5-yl)-[1,2]thiazolo[4,5-b]pyridin-5-yl]-3-methylmorpholine

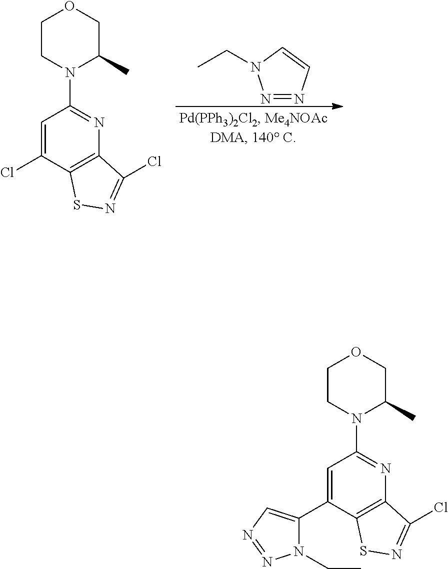

A mixture of (3R)-4-{3,7-dichloro-[1,2]thiazolo[4,5-b]pyridin-5-yl}-3-methylmorpholine (200 mg, 0.657 mmol), 1-ethyl-1H-1,2,3-triazole (383.12 mg, 3.945 mmol), tetramethylammonium acetate (262.70 mg, 1.972 mmol) and Pd(PPh₃)₂Cl₂ (92.29 mg, 0.131 mmol) in DMA (3 mL) was stirred at 140° C. for 8 hrs under nitrogen atmosphere. The reaction mixture was diluted with water and extracted with ethyl acetate. The organic layer was washed with brine, dried over Na₂SO₄, filtered and concentrated in vacuo. The residue was purified on flash column chromatography (Silica, 0˜100% ethyl acetate in petroleum ether) to give the title product (3R)-4-[3-chloro-7-(1-ethyl-1H-1,2,3-triazol-5-yl)-[1,2]thiazolo[4,5-b]pyridin-5-yl]-3-methylmorpholine (130 mg, 0.356 mmol, 54.19%). LC-MS(ESI+): m/z (M+H)=364.8, 366.8

### Step 2. (3R)-4-[7-(1-ethyl-1H-1,2,3-triazol-5-yl)-3-[3-methyl-1-(oxan-2-yl)-1H-pyrazol-5-yl]-[1,2]thiazolo[4,5-b]pyridin-5-yl]-3-methylmorpholine

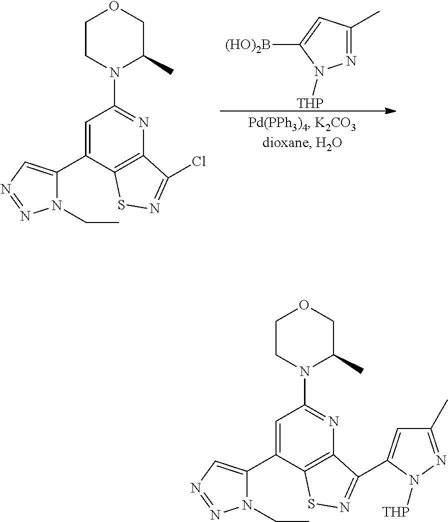

A mixture of (3R)-4-[3-chloro-7-(1-ethyl-1H-1,2,3-triazol-5-yl)-[1,2]thiazolo[4,5-b]pyridin-5-yl]-3-methylmorpholine (130 mg, 0.356 mmol), [3-methyl-1-(oxan-2-yl)-1H-pyrazol-5-yl]boronic acid (224.51 mg, 1.069 mmol), Pd(PPh₃)₄ (82.28 mg, 0.071 mmol) and K₂CO₃ (3 mL, 6.000 mmol) in dioxane (15 mL) and water (3 mL) was stirred overnight at 100° C. under nitrogen atmosphere. The reaction mixture was diluted with water and extracted with ethyl acetate. The combined organic layer was washed with brine, dried over Na₂SO₄, filtered and concentrated in vacuo. The residue was purified on flash column chromatography (Silica, 0˜100% Ethyl Acetate in petroleum ether) to give the title product (3R)-4-[7-(1-ethyl-1H-1,2,3-triazol-5-yl)-3-[3-methyl-1-(oxan-2-yl)-1H-pyrazol-5-yl]-[1,2]thiazolo[4,5-b]pyridin-5-yl]-3-methylmorpholine (74 mg, 0.150 mmol, 41.99%). LC-MS(ESI+): m/z (M+H)=494.8

### Step 3. (3R)-4-[7-(1-ethyl-1H-1,2,3-triazol-5-yl)-3-(3-methyl-1H-pyrazol-5-yl)-[1,2]thiazolo[4,5-b]pyridin-5-yl]-3-methylmorpholine

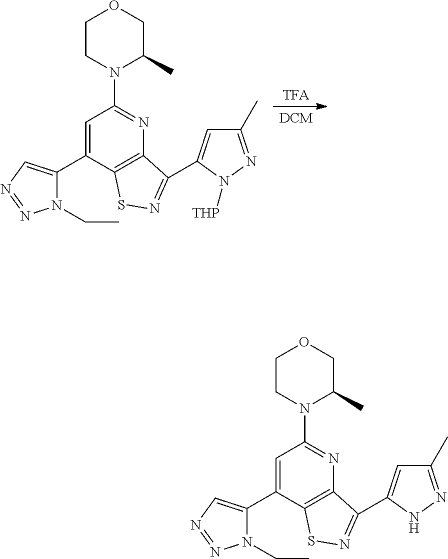

To a solution of (3R)-4-[7-(1-ethyl-1H-1,2,3-triazol-5-yl)-3-[3-methyl-1-(oxan-2-yl)-1H-pyrazol-5-yl]-[1,2]thiazolo[4,5-b]pyridin-5-yl]-3-methylmorpholine (74 mg, 0.150 mmol) in DCM (2 mL) was added TFA (2 mL) and the resulting mixture was stirred for 1 h at ambient temperature. The mixture was concentrated and basified with saturated ammonium. The mixture was concentrated and the residue was purified on flash column chromatography (Silica, 0˜10% MeOH in DCM) and Prep-HPLC (C18, 10-95%, acetonitrile in water with 0.1% formic acid) to give the title product (3R)-4-[7-(1-ethyl-1H-1,2,3-triazol-5-yl)-3-(3-methyl-1H-pyrazol-5-yl)-[1,2]thiazolo[4,5-b]pyridin-5-yl]-3-methylmorpholine (16.7 mg, 0.041 mmol, 27.19%). LC-MS(ESI+): m/z (M+H)=410.9. ¹H NMR (400 MHz, DMSO) δ 13.13 (d, J=125.0 Hz, 1H), 8.21 (s, 1H), 7.46 (s, 1H), 7.17 (s, 1H), 4.52 (dd, J=14.5, 7.2 Hz, 3H), 4.18 (d, J=12.5 Hz, 1H), 4.05 (d, J=10.2 Hz, 1H), 3.83 (d, J=11.3 Hz, 1H), 3.74 (d, J=10.8 Hz, 1H), 3.59 (t, J=10.7 Hz, 1H), 3.27 (d, J=11.4 Hz, 1H), 2.31 (s, 3H), 1.38 (t, J=7.2 Hz, 3H), 1.26 (d, J=6.5 Hz, 3H).

### Example 108

### Synthesis of dimethyl(3-(3-methyl-1-(tetrahydro-2H-pyran-2-yl)-1H-pyrazol-5-yl)-5-((R)-3-methylmorpholino)isothiazolo[4,5-b]pyridin-7-yl)phosphine oxide

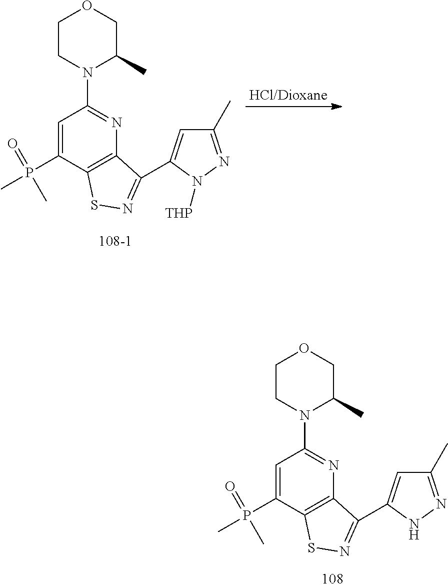

### Step 1. dimethyl(3-(3-methyl-1-(tetrahydro-2H-pyran-2-yl)-1H-pyrazol-5-yl)-5-((R)-3-methylmorpholino)isothiazolo[4,5-b]pyridin-7-yl)phosphine oxide

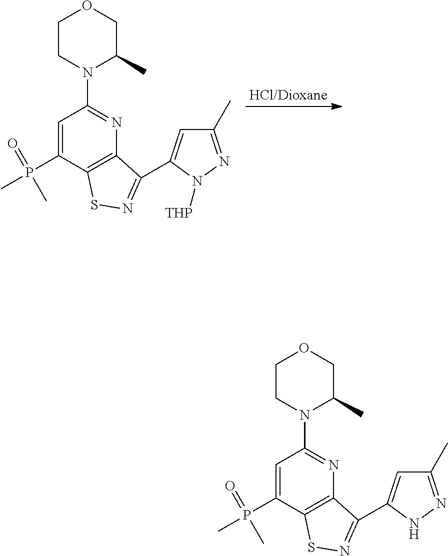

A solution of dimethyl(3-(3-methyl-1-(tetrahydro-2H-pyran-2-yl)-1H-pyrazol-5-yl)-5-((R)-3-methylmorpholino)isothiazolo[4,5-b]pyridin-7-yl)phosphine oxide (15 mg, 0.032 mmol) in HCl/Dioxane (4M) (1 mL) was stirred at room temperature for 1 hr. The reaction mixture was concentrated in vacuo. The residue was purified by prep-HPLC (C₁₈, 10-95%, MeOH in H₂O with 0.1% HCOOH) to afford the desired product (4 mg, 0.010 mmol, 32.39%). LC/MS (ESI) m/z: 476 (M+H)⁺. ¹H NMR (400 MHz, DMSO) δ 13.03 (d, J=124.9 Hz, 1H), 7.49 (d, J=13.6 Hz, 1H), 7.10 (s, 1H), 4.58 (s, 1H), 4.15 (d, J=12.8 Hz, 1H), 4.05 (d, J=11.3 Hz, 1H), 3.83 (d, J=11.4 Hz, 1H), 3.73 (d, J=12.2 Hz, 1H), 3.58 (t, J=11.3 Hz, 1H), 3.25 (d, J=10.4 Hz, 1H), 2.30 (s, 3H), 1.85 (d, J=13.8 Hz, 6H), 1.24 (d, J=6.2 Hz, 3H).

### Example 109

### Synthesis of (R)-4-(5-fluoro-4-(1-methyl-1H-pyrazol-5-yl)-7-(1H-pyrazol-5-yl)imidazo[1,5-b]pyridazin-2-yl)-3-methylmorpholine

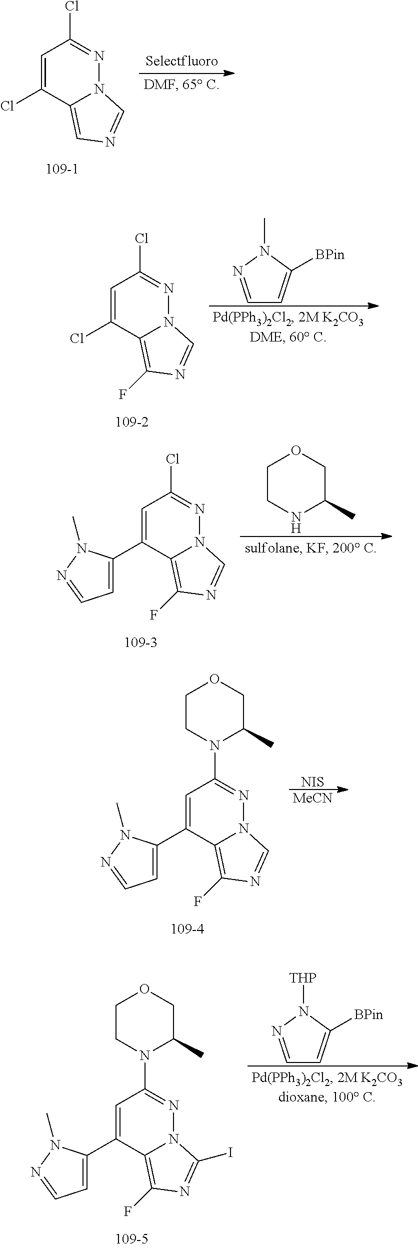

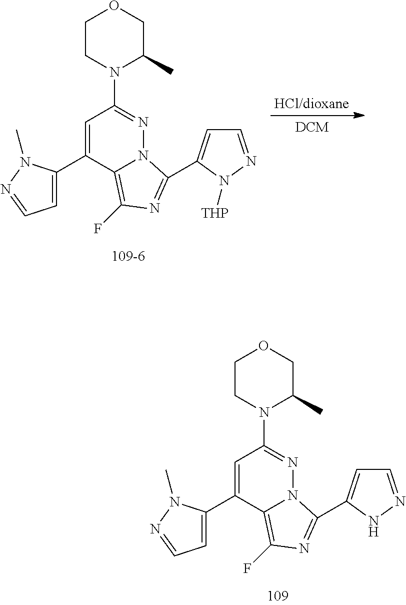

### Step 1. 2,4-dichloro-5-fluoroimidazo[1,5-b]pyridazine

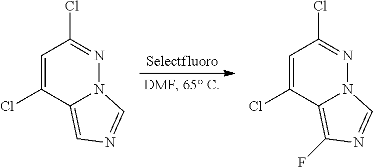 [SMILES: Clc1cc(Cl)c2cncn2n1 ; Fc1ncn2nc(Cl)cc(Cl)c12]

To a solution of 2,4-dichloroimidazo[1,5-b]pyridazine (607 mg, 3.228 mmol) in DMF (40 mL) was added selectfluor (2287.37 mg, 6.457 mmol) and the resulting mixture was stirred overnight at 65° C. under nitrogen atmosphere. After quenched with saturated NaHCO₃, the mixture was extracted with ethyl acetate and the organic layer was washed with brine, dried over Na₂SO₄, filtered and concentrated in vacuo. The residue was purified on flash column chromatography (Silica, 0˜10% ethyl acetate in petroleum ether) to give the title product 2,4-dichloro-5-fluoroimidazo[1,5-b]pyridazine (124 mg, 0.602 mmol, 18.64%). LC-MS(ESI+): m/z (M+H)=205.8, 207.8 Step 2. 2-chloro-5-fluoro-4-(1-methyl-1H-pyrazol-S-yl)imidazo[1,5-b]pyridazine

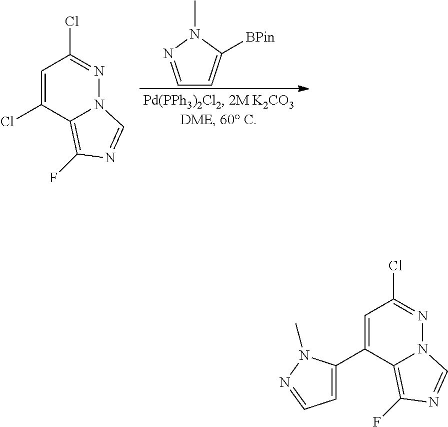

A mixture of 2,4-dichloro-5-fluoroimidazo[1,5-b]pyridazine (124 mg, 0.602 mmol), 1-methyl-5-(tetramethyl-1,3,2-dioxaborolan-2-yl)-1H-pyrazole (131.50 mg, 0.632 mmol), Pd(PPh₃)₂Cl₂ (42.25 mg, 0.060 mmol) and Na₂CO₃ (191.39 mg, 1.806 mmol) in DME (10 mL) and H₂O (1 mL) was stirred overnight at 60° C. under nitrogen atmosphere. The reaction mixture was concentrated in vacuo and the residue was purified on flash column chromatography (Silica, 0˜40% Ethyl Acetate in petroleum ether) to give the title product 5-{2-chloro-5-fluoroimidazo[1,5-b]pyridazin-4-yl}-1-methyl-1H-pyrazole (110 mg, 0.437 mmol, 72.62%). LC-MS(ESI+): m/z (M+H)=251.9, 253.9.

### Step 3. (R)-4-(5-fluoro-4-(1-methyl-1H-pyrazol-5-yl)imidazo[1,5-b]pyridazin-2-yl)-3-methylmorpholine

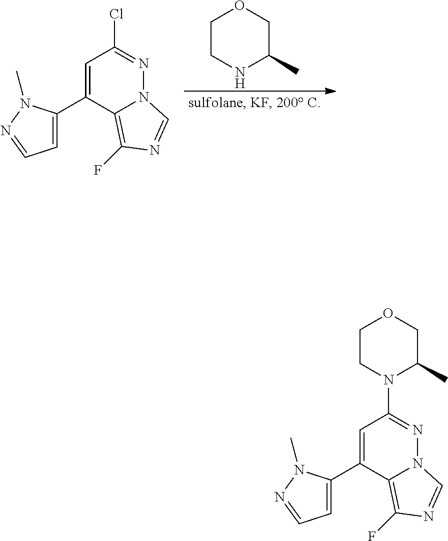

A mixture of 5-{2-chloro-5-fluoroimidazo[1,5-b]pyridazin-4-yl}-1-methyl-1H-pyrazole (110 mg, 0.437 mmol), (3R)-3-methylmorpholine (132.61 mg, 1.311 mmol) and potassium fluoride (0.031 mL, 1.311 mmol) in sulfolane (2 mL) was stirred at 200° C. under nitrogen atmosphere for 8 hrs in a sealed tube. The reaction mixture was purified on flash column chromatography (Silica, 0˜10% MeOH in DCM) and Prep-HPLC (C18, 10-95% acetonitrile in water with 0.1% formic acid) to give the title product (3R)-4-[5-fluoro-4-(1-methyl-1H-pyrazol-5-yl)imidazo[1,5-b]pyridazin-2-yl]-3-methylmorpholine (56 mg, 0.177 mmol, 40.50%). LC-MS(ESI+): m/z (M+H)=316.9

### Step 4. 2-chloro-5-fluoro-4-(1-methyl-1H-pyrazol-5-yl)imidazo[1,5-b]pyridazine

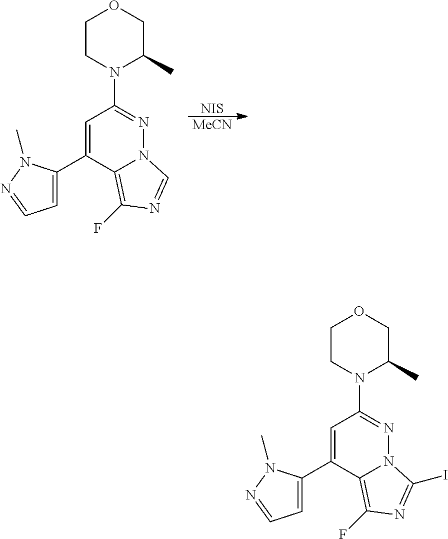

To a solution of (3R)-4-[5-fluoro-4-(1-methyl-1H-pyrazol-5-yl)imidazo[1,5-b]pyridazin-2-yl]-3-methylmorpholine (56 mg, 0.177 mmol) in acetonitrile (10 mL) was added NIS (47.79 mg, 0.212 mmol) and the resulting mixture was stirred at ambient temperature under nitrogen atmosphere for 1.5 hrs. The reaction mixture was quenched with saturated NaHCO₃ and saturated Na₂S₂O₃ and extracted with ethyl acetate. The organic layer was washed with water twice and brine, dried over Na₂SO₄, filtered and concentrated. The residue was purified on flash column chromatography (Silica, 0˜100% Ethyl Acetate in petroleum ether) to give the title product (3R)-4-[5-fluoro-7-iodo-4-(1-methyl-1H-pyrazol-5-yl)imidazo[1,5-b]pyridazin-2-yl]-3-methylmorpholine (35 mg, 0.079 mmol, 44.71%). LC-MS(ESI+): m/z (M+H)=442.7

### Step 5. (3R)-4-(5-fluoro-4-(1-methyl-1H-pyrazol-5-yl)-7-(1-(tetrahydro-2H-pyran-2-yl)-1H-pyrazol-5-yl)imidazo[1,5-b]pyridazin-2-yl)-3-methylmorpholine

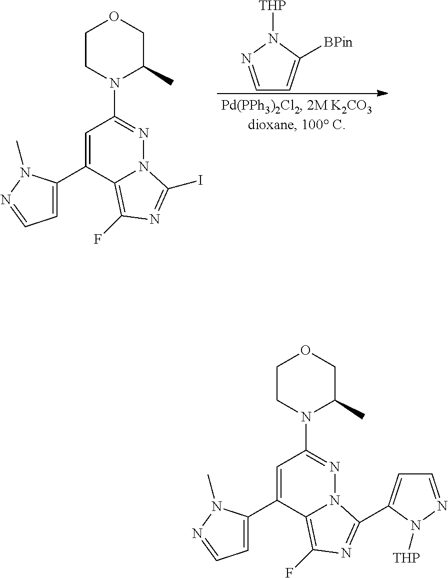

A mixture of (3R)-4-[5-fluoro-7-iodo-4-(1-methyl-1H-pyrazol-5-yl)imidazo[1,5-b]pyridazin-2-yl]-3-methylmorpholine (35 mg, 0.079 mmol), 1-(oxan-2-yl)-5-(tetramethyl-1,3,2-dioxaborolan-2-yl)-1H-pyrazole (55.04 mg, 0.198 mmol), Pd(PPh₃)₂Cl₂ (5.56 mg, 0.008 mmol) and K₂CO₃ (32.81 mg, 0.237 mmol) in dioxane (10 mL) and water (1 mL) was stirred overnight at 100° C. under nitrogen atmosphere. The reaction mixture was concentrated in vacuo and the residue was purified on flash column chromatography (Silica, 0˜10% MeOH in DCM) to give the title product (3R)-4-[5-fluoro-4-(1-methyl-1H-pyrazol-5-yl)-7-[1-(oxan-2-yl)-1H-pyrazol-5-yl]imidazo[1,5-b]pyridazin-2-yl]-3-methylmorpholine (18 mg, 0.039 mmol, 48.75%). LC-MS(ESI+): m/z (M+H)=466.9.

### Step 6. (R)-4-(5-fluoro-4-(1-methyl-1H-pyrazol-5-yl)-7-(1H-pyrazol-5-yl)imidazo[1,5-b]pyridazin-2-yl)-3-methylmorpholine

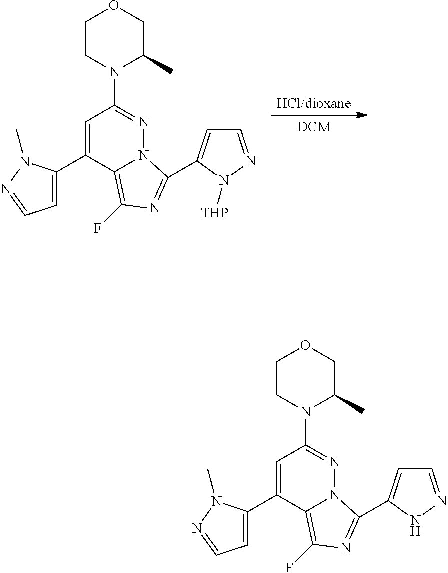

To a solution of (3R)-4-[5-fluoro-4-(1-methyl-1H-pyrazol-5-yl)-7-[1-(oxan-2-yl)-1H-pyrazol-5-yl]imidazo[1,5-b]pyridazin-2-yl]-3-methylmorpholine (18 mg, 0.039 mmol) in DCM (2 mL) was added HCl/dioxane (4M, 2 mL) and the resulting mixture was stirred for 1 h at ambient temperature. The mixture was concentrated and basified with saturated ammonium. The mixture was concentrated and the residue was purified on flash column chromatography (Silica, 0˜10% MeOH in DCM) and Prep-HPLC (C18, 10-95%, acetonitrile in water with 0.1% formic acid) to give the title product (3R)-4-[5-fluoro-4-(1-methyl-1H-pyrazol-5-yl)-7-(1H-pyrazol-5-yl)imidazo[1,5-b]pyridazin-2-yl]-3-methylmorpholine (4.9 mg, 0.013 mmol, 33.21%). LC-MS(ESI+): m/z (M+H)=407.9. ¹H NMR (400 MHz, DMSO) δ 13.37 (d, J=122.0 Hz, 1H), 7.78 (d, J=85.7 Hz, 1H), 7.62 (d, J=1.9 Hz, 1H), 7.12 (s, 1H), 6.90 (s, 1H), 6.64 (s, 1H), 4.39 (s, 1H), 4.00 (dd, J=11.4, 3.1 Hz, 1H), 3.94 (d, J=15.1 Hz, 4H), 3.77 (d, J=11.5 Hz, 1H), 3.71 (dd, J=11.5, 2.7 Hz, 1H), 3.56 (td, J=11.8, 2.8 Hz, 1H), 3.28 (d, J=13.1 Hz, 1H), 1.27 (d, J=6.7 Hz, 3H).

### Example 110

### Biochemical Assays

Assay 1: ATR Inhibition Assay

Detection of ATR kinase activity utilized the Mobility shift assay to measure the phosphorylation of the substrate protein FAM-RAD17 (GL, Cat. No. 514318, Lot. No. P19042-MJ524315). The assay was developed and conducted at Chempartner. All the test compounds were dissolved in 100% DMSO at concentration of 20 mM, then prepare compounds and conducted the assay as follows:
1) Transfer 80 μl 20 mM compound to 40 μl of 100% DMSO in a 96-well plate.
2) Serially dilute the compound by transferring 20 μl to 60 μl of 100% DMSO in the next well and so forth for a total of 10 concentrations.
3) Add 100 μl of 100% DMSO to two empty wells for no compound control and no enzyme control in the same 96-well plate. Mark the plate as source plate.
4) Transfer 40 μl of compound from source plate to a new 384-well plate as the intermediate plate.
5) Transfer 60 nl compounds to assay plate by Echo.
6) Add ATR kinase (Eurofins, Cat. No. 14-953, Lot. No. D14 JP007N) into Kinase base buffer (50 mM HEPES, pH 7.5; 0.0015% Brij-35; 0.01% Triton) to prepare 2× enzyme solution, then add 10 μl of 2× enzyme solution to each well of the 384-well assay plate, incubate at room temperature for 10 min.
7) Add FAM-RAD17 and ATP (Sigma, Cat. No. A7699-1G, CAS No. 987-65-5) in the kinase base buffer to prepare 2×peptide solution, then add 10 μl to the assay plate.
8) Incubate at 28° C. for specified period of time. Add 40 μl of stop buffer (100 mM HEPES, pH 7.5; 0.015% Brij-35; 0.2% Coating Reagent #3; 50 mM EDTA) to stop reaction.
9) Collect data on Caliper. Convert conversion values to inhibition values.

Percent inhibition=(max−conversion)/(max−min)*100

wherein “max” stands for DMSO control; “min” stands for low control.

Fit the data in XLFit excel add-in version 5.4.0.8 to obtain IC50 values. Equation used is:

Y=Bottom+(Top−Bottom)/(1+(IC50/X)\hat{ }HillSlope)

wherein X means concentration in a format not transformed to logarithms.

The following Table 2 lists the IC₅₀ values for exemplary compounds of Formula (I).

TABLE 2

| Compd. | ATR |
|---|---|
| No. | IC₅₀ (nM) |
| 1 | A |
| 2 | C |
| 3 | B |
| 4 | A |
| 5 | A |
| 6 | A |
| 7a | A |
| 7b | A |
| 8 | A |
| 9 | A |
| 10 | A |
| 11 | A |
| 14 | C |
| 18 | A |
| 21 | A |
| 22 | A |
| 27 | A |
| 29 | A |
| 30 | A |
| 31 | A |
| 32 | A |
| 33 | A |
| 34 | A |
| 39 | A |
| 48 | A |
| 50 | A |
| 51 | A |
| 52 | A |
| 53 | A |
| 55 | A |
| 56 | A |
| 57 | A |
| 58 | A |
| 60 | A |
| 61 | A |
| 62 | A |
| 63 | A |
| 64 | A |
| 75 | A |
| 76 | A |
| 80 | A |
| 81 | A |
| 82 | A |
| 83 | A |
| 84 | A |
| 85 | A |
| 87 | A |
| 89 | A |

A: IC₅₀ < 100 nM; B: 100 nM ≤ IC₅₀ ≤ 500 nM; C: IC₅₀ > 500 nM

For the other compounds provided herein for which the results are not shown, all have an IC₅₀ against ATR kinase of no more than 1000 nM. Some of these compounds have an IC₅₀ against ATR kinase of no more than 500 nM, some no more than 400 nM, some no more than 300 nM, some no more than 200 nM, or no more than 100 nM, or even no more than 50 nM.

Therefore, as determined by ATR inhibition assay, the compounds of the present disclosure have a good inhibitory effect on ATR kinase activity.

Assay 2: Tumor Cell Anti-proliferation Assay (CTG Assay)

Human colorectal cancer cells HT-29 (HTB-38) and LoVo (CCL-229) were selected for the CTG assay, the two cell lines were originally obtained from the American Type Culture Collection (ATCC). FBS and appropriate additives were added into base medium to prepare complete medium, then the cell layer was briefly rinsed with 0.25% (w/v) Trypsin-0.038% (w/v) EDTA solution to remove all traces of serum that contains trypsin inhibitor. After that, appropriate volume of Trypsin-EDTA solution was added to flask and cells under an inverted microscope were observed until cell layer was dispersed. At last, appropriate volume of complete growth medium was added and cells were aspirated by gently pipetting. Numbers were collected and counted with Vi-cell XR and cell density was adjusted, cells were seeded into 96-well opaque-walled clear bottom tissue-culture treated plates in the CO₂ incubator for 20-24 hours. All the test compounds were at 10 mM in DMSO. Compounds were then added to the cell media in 3-fold serial dilutions, the final DMSO concentration was 0.5%. Plates were incubated for 96 h at 5% CO₂, 37° C. Before the measurement, the appropriate volume of CellTiter-Glo Buffer was transferred into the amber bottle containing CellTiter-Glo substrate to reconstitute the lyophilized enzyme/substrate mixture, mixed gently, thereby forming the CellTiter-Glo Reagent (Promega Cat. No. G7573). The plate and its contents were equilibrated to room temperature for approximately 30 minutes, then 100 μL of CellTiter-Glo Reagent was added to the assay plate, the contents were mixed for 2 minutes on an orbital shaker to induce cell lysis, incubated at room temperature for 10 minutes to stabilize luminescent signal. At last the clear bottom was pasted with white back seal and luminescence was recorded with Enspire. IC₅₀ and GI₅₀ values were calculated with XLFit curve fitting software using 4 Parameter Logistic Model Y=Bottom+(Top−Bottom)/(1+(IC50/X)\hat{ }HillSlope).

The following Table 3 provides the IC₅₀ values for exemplary compounds of Formula (I).

TABLE 3

| Compound | LoVo |
|---|---|
| No. | IC₅₀ (nM) |
| 1 | 491 |
| 5 | 516 |
| 6 | 803 |
| 11 | 384 |
| 18 | 384 |
| 22 | 622 |
| 23 | 446 |
| 25 | 550 |
| 27 | 67 |
| 28 | 471 |
| 29 | 14 |
| 30 | 152 |
| 31 | 84 |
| 32 | 553 |
| 33 | 162 |
| 34 | 23 |
| 35 | 129 |
| 36 | 17.8 |
| 37 | 36.6 |
| 38 | 40.4 |
| 40 | 23.8 |
| 41 | 86.6 |
| 42 | 79.3 |
| 44 | 43.9 |
| 45 | 81.7 |
| 46 | 51.2 |
| 47 | 17.1 |
| 48 | 33.3 |
| 49 | 16.1 |
| 50 | 17.0 |
| 51 | 15.9 |
| 52 | 17.7 |
| 53 | 12.8 |
| 54 | 66.7 |
| 55 | 13.7 |
| 56 | 19.0 |
| 57 | 2.5 |
| 58 | <1 |
| 59 | 28.1 |
| 60 | 4.1 |
| 61 | 26.5 |
| 62 | 14.6 |
| 63 | 38.9 |
| 64 | 15.1 |
| 65 | 55.2 |
| 66 | 69.0 |
| 67 | 5 |
| 68 | 16 |
| 69 | 15 |
| 70 | 18 |
| 71 | 23 |
| 72 | 101 |
| 73 | 41 |
| 74 | 5 |
| 75 | 15.3 |
| 76 | 24.3 |
| 77 | 1000 |
| 78 | 152 |
| 79 | 246 |
| 80 | 10 |
| 81 | 10 |
| 82 | 20 |
| 83 | 14 |
| 84 | 9 |
| 85 | 4 |
| 86 | 9 |
| 87 | 3 |
| 88 | 1 |
| 89 | <1 |
| 90 | 5 |
| 91 | 2 |
| 92 | 94 |
| 94 | 12 |
| 95 | 36 |
| 96 | 32.7 |
| 97 | 10 |
| 98 | 14 |
| 99 | 16 |
| 100 | 16 |
| 101 | 15.5 |
| 102 | 26 |
| 103 | 15 |
| 104 | 154 |
| 105 | 9 |
| 106 | 46 |
| 107 | 15 |
| 108 | 35 |

The foregoing description is considered as illustrative only of the principles of the present disclosure. Further, since numerous modifications and changes will be readily apparent to those skilled in the art, it is not desired to limit the invention to the exact construction and process shown as described above. Accordingly, all suitable modifications and equivalents may be considered to fall within the scope of the invention as defined by the claims that follow.

1. A compound having Formula (I′): 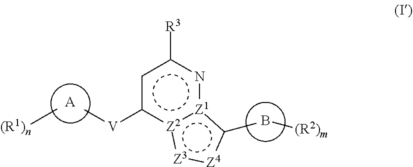
or a pharmaceutically acceptable salt thereof,
wherein
Z¹ is C or N;
Z² is C or N;
Z³ is CR^{d}, N, O, S, S(O) or S(O)₂;
Z⁴ is CH or N;
V is a direct bond, or alkyl optionally substituted with one or more R^{e} or —N(R^{a})—;
Ring A is absent, 3- to 6-membered cycloalkyl, 5- to 6-membered heterocyclyl, 5- to 6-membered aryl, or 5- to 6-membered heteroaryl;
R¹, in each occurrence, is selected from the group consisting of hydrogen, hydroxyl, halogen, cyano, alkyl, haloalkyl, hydroxylalkyl, —C(O)N(R^{a})₂, —C(O)OR^{a}, —S(O)₂(R^{b}), —S(O)(NH)(R^{b}) and —P(O)(R^{b})₂;
Ring B is 5- to 6-membered heterocyclyl or 5- to 6-membered heteroaryl;
R², in each occurrence, is halogen, alkyl, haloalkyl, or cycloalkyl;
R³ is or; 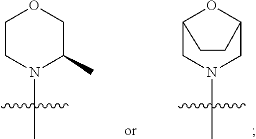
R^{a} and R^{d} are each independently hydrogen, halogen or alkyl;
R^{b} is alkyl, 3- to 6-membered cycloalkyl, 5- to 6-membered heterocyclyl, 5- to 6-membered aryl, or 5- to 6-membered heteroaryl, wherein said cycloalkyl, heterocyclyl, and heteroaryl are optionally substituted with one or more R^{c};
R^{c} is selected from the group consisting of hydroxyl, halogen, cyano, amino, alkyl, alkoxyl, and haloalkyl;
R^{c} is hydroxyl, halogen or alkyl;
n is 0, 1, 2, or 3; and
m is 0, 1, 2 or 3.

2. A compound having Formula (I): 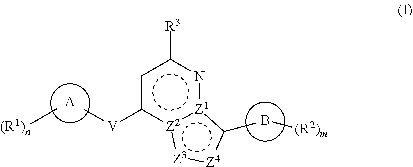
or a pharmaceutically acceptable salt thereof,
wherein
Z¹ is C or N;
Z² is C or N;
Z³ is CH, N, or S;
Z⁴ is CH or N;
V is a direct bond or —N(R^{a})—;
Ring A is absent, 3- to 6-membered cycloalkyl, 5- to 6-membered heterocyclyl, 5- to 6-membered aryl, or 5- to 6-membered heteroaryl;
R¹ is hydrogen, halogen, alkyl, —S(O)₂(R^{b}), or —S(O)(NH)(R^{b});
Ring B is 5- to 6-membered heterocyclyl or 5- to 6-membered heteroaryl;
R² is halogen, alkyl, haloalkyl, or cycloalkyl;
R³ is or; 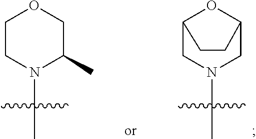
R^{a} is hydrogen or alkyl;
R^{b} is alkyl, 3- to 6-membered cycloalkyl, 5- to 6-membered heterocyclyl, 5- to 6-membered aryl, or 5- to 6-membered heteroaryl, wherein said cycloalkyl, heterocyclyl, and heteroaryl are optionally substituted with one or more R^{c};
R^{c} is selected from the group consisting of hydroxyl, halogen, cyano, amino, alkyl, alkoxyl, and haloalkyl;
n is 0, 1, 2, or 3; and
m is 0, 1, 2 or 3.

3-6. (canceled)

7. The compound of claim 1 or 2, or a pharmaceutically acceptable salt thereof, wherein Z¹ and Z² are selected from any one of the following:
(1) Z¹ is C and Z² is N,
(2) Z¹ is N and Z² is C; or
(3) Z¹ is C and Z² is C.

8-17. (canceled)

18. The compound of claim 1 or 2, or a pharmaceutically acceptable salt thereof, wherein Z¹, Z² and Z³ are selected from any one of the following:
(1) Z¹ is C, Z² is N and is CH or N;
(2) Z¹ is N, Z² is C and Z³ is CH, C(CH₃) or N; or
(3) Z¹ is C, Z² is C and Z³ is O, S, S(O) or S(O).

19-35. (canceled)

36. The compound of claim 1 or 2, or a pharmaceutically acceptable salt thereof, wherein Ring A is selected from the group consisting of:

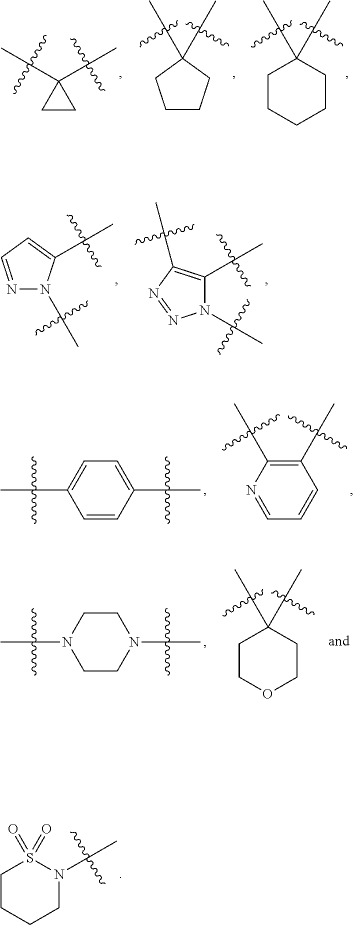

37. (canceled)

38. The compound of claim 1 or 2, or a pharmaceutically acceptable salt thereof, wherein R¹ is hydrogen, cyano, fluoro, hydroxyl, C₁₋₃ alkyl, C₁₋₃ haloalkyl, C₁₋₃ hydroxylalkyl, —C(O)N(R^{a})₂, —C(O)OR^{a}, —S(O)₂(R^{b}), S(O)(NH)(R^{b}) or —P(O)(R^{b})₂.

39-45. (canceled)

46. The compound of claim 1 or 2, or a pharmaceutically acceptable salt thereof, wherein Ring A is pyrazolyl, pyridyl or triazolyl, and R¹ is halogen or alkyl.

47. (canceled)

48. The compound of claim 1 or 2, or a pharmaceutically acceptable salt thereof, wherein Ring A is cyclopropyl, cyclopentyl, cyclohexyl, piperazinyl or phenyl, and R¹ is cyano, —(O)₂(R^{b}), or —S(O)(NH)(R^{b}).

49-61. (canceled)

62. The compound of claim 1 or 2, or a pharmaceutically acceptable salt thereof, wherein 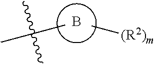
is selected from the group consisting of:

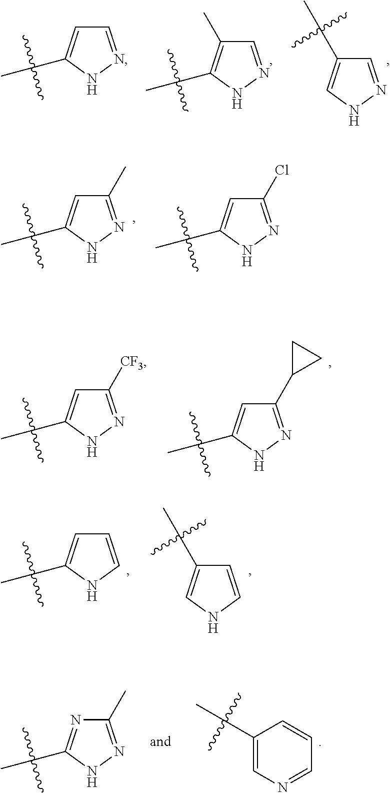

63-64. (canceled)

65. The compound of claim 1 or 2, having a formula selected from the group consisting of: 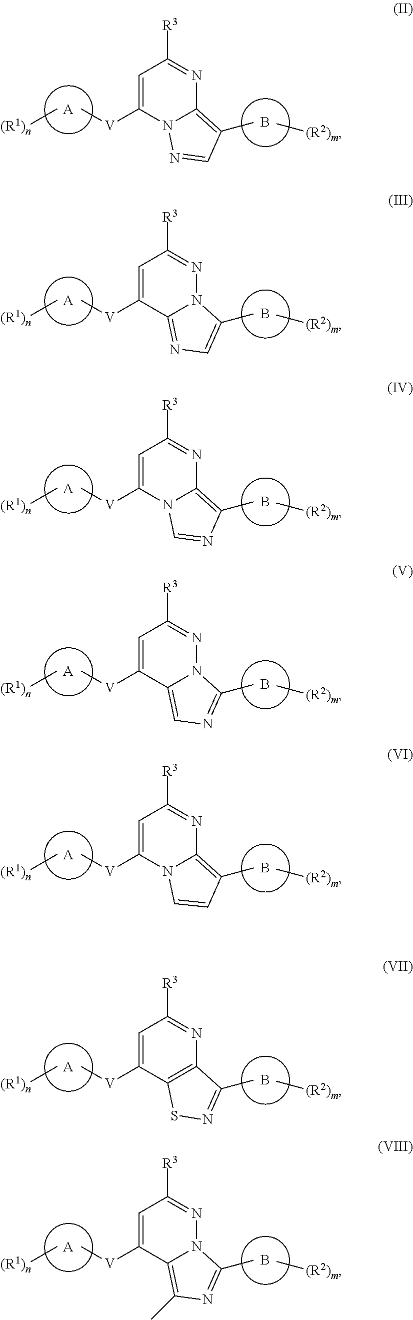 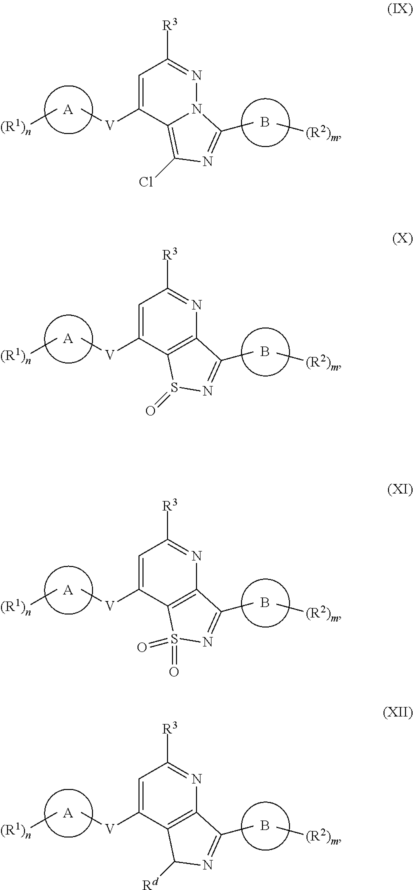 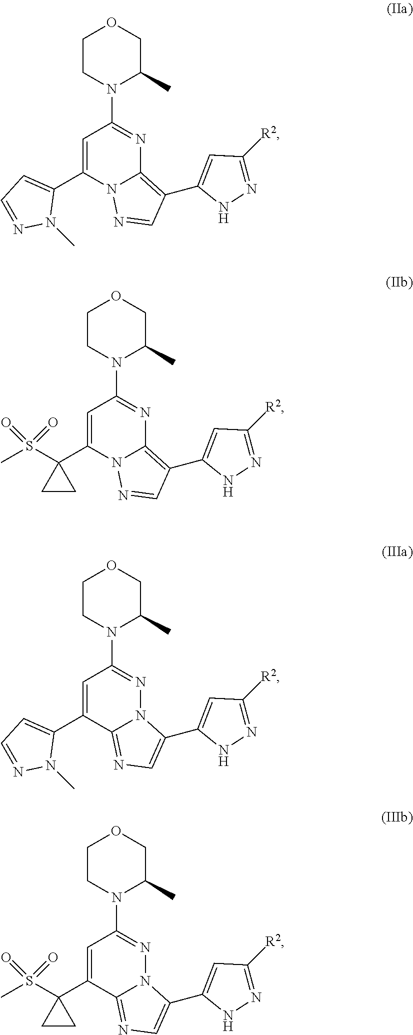 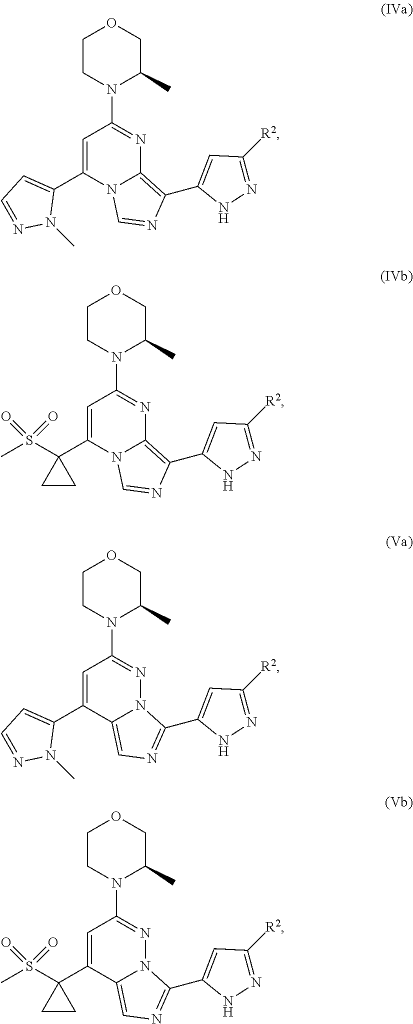 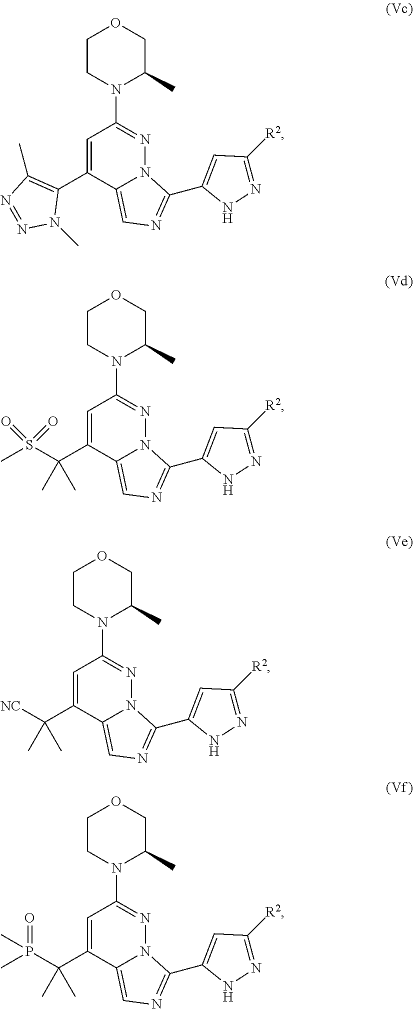 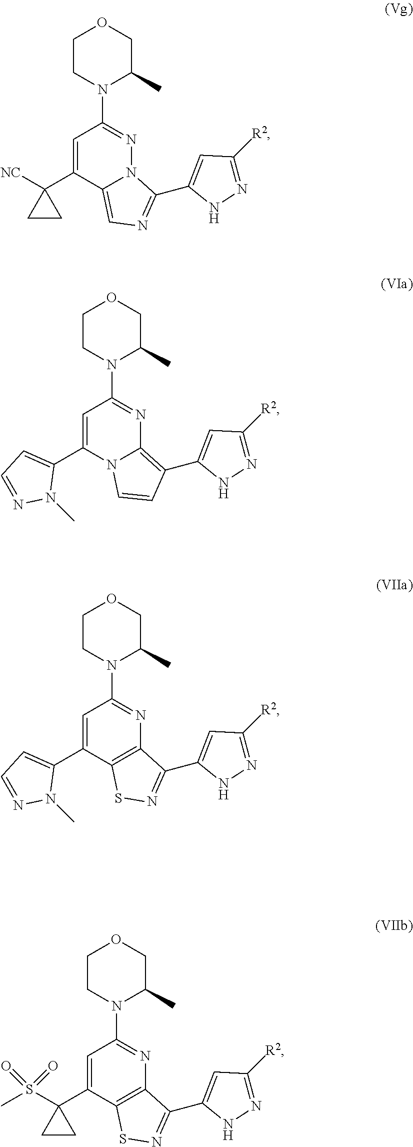 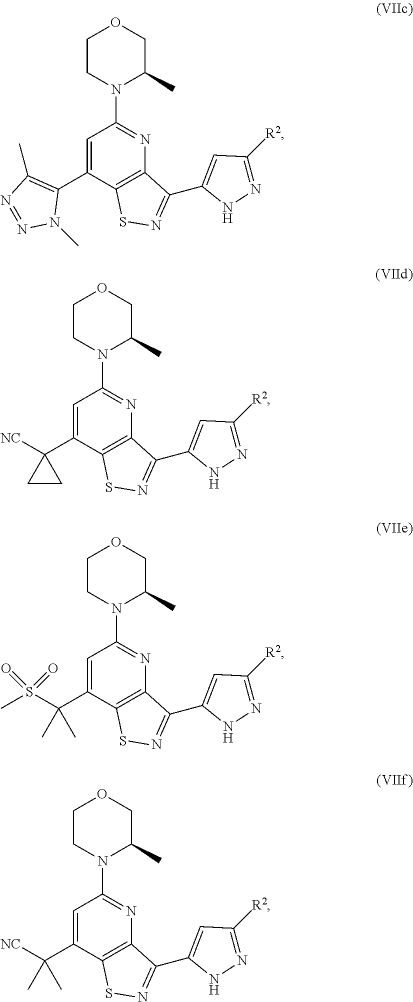 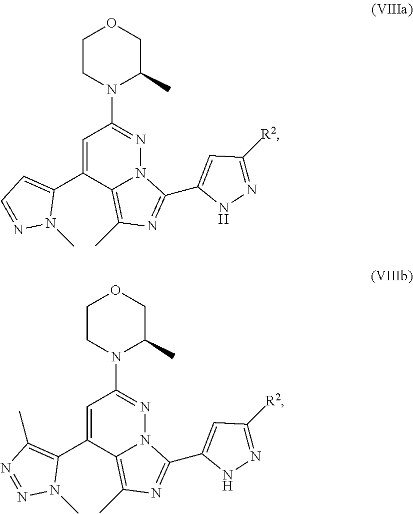 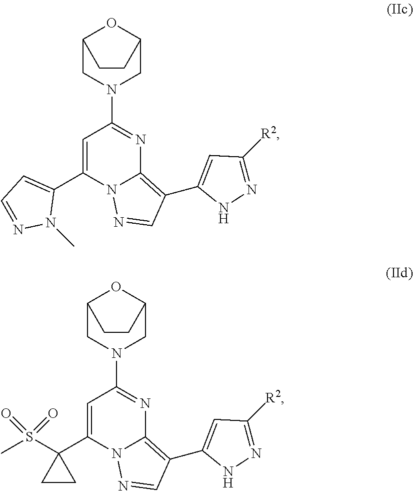 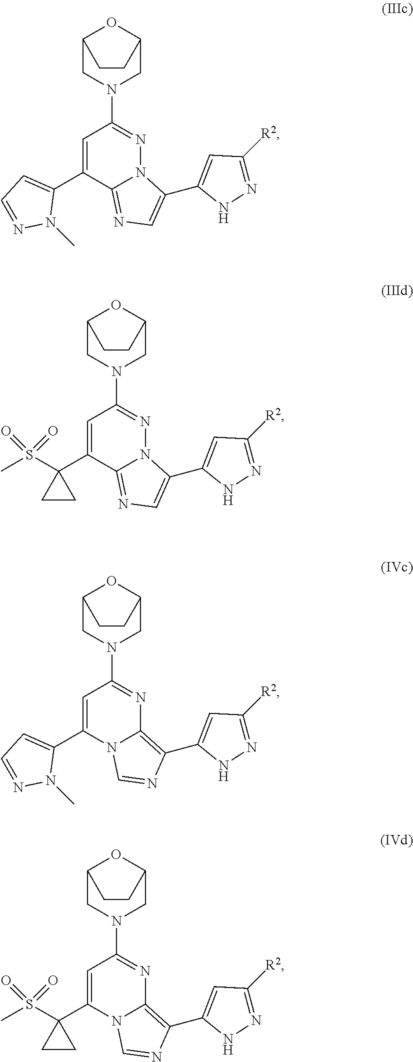 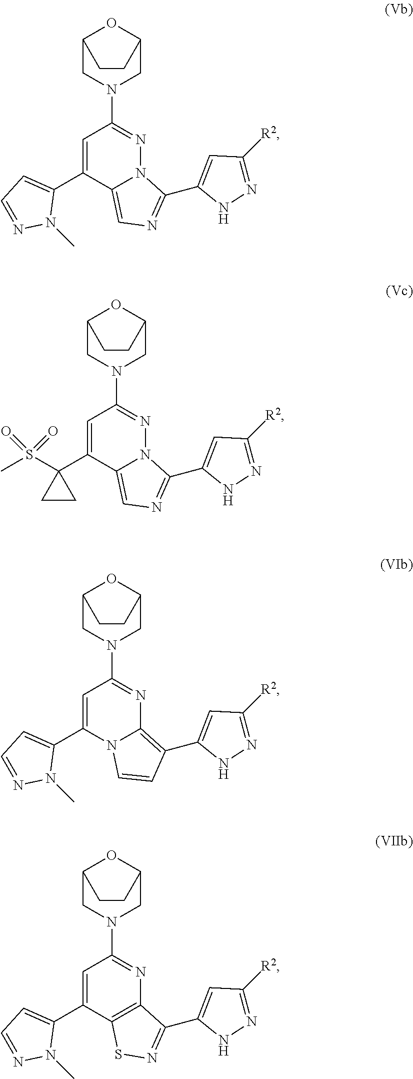
or a pharmaceutically acceptable salt thereof.

66. The compound of claim 65, or a pharmaceutically acceptable salt thereof, wherein:
V is a direct bond or C₁₋₃ alkyl;
Ring A is selected from cyclopropyl, cyclopentyl, cyclohexyl, piperazinyl, phenyl, pyrazolyl, pyridinyl, or triazolyl;
R¹ is selected from hydrogen, hydroxyl, fluoro, cyano, methyl, —S(O)₂(R^{b}), —S(O)(NH)(R^{b}) or —P(O)(R^{b})₂;
Ring B is pyrazolyl, pyrrolyl, or pyridyl;
R² is chloro, C₁₋₃ alkyl, C₁₋₃ haloalkyl, or 3- to 6-membered cycloalkyl;
R³ is 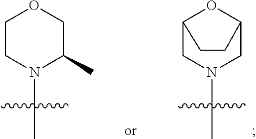
R^{b} is C₁₋₃ alkyl;
R^{d} is hydrogen, chloro or C₁₋₃ alkyl;
n is 0, 1 or 2; and
m is 0, 1 or 2.

67-68. (canceled)

69. A compound selected from the group consisting of: 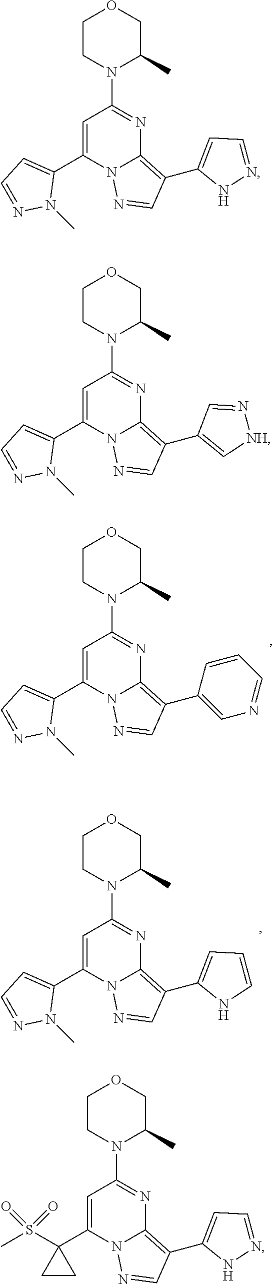 [SMILES: C[C@@H]1COCCN1c1cc(-c2ccnn2C)n2ncc(-c3ccn[nH]3)c2n1 ; C[C@@H]1COCCN1c1cc(-c2ccnn2C)n2ncc(-c3cn[nH]c3)c2n1 ; C[C@@H]1COCCN1c1cc(-c2ccnn2C)n2ncc(-c3cccnc3)c2n1 ; C[C@@H]1COCCN1c1cc(-c2ccnn2C)n2ncc(-c3ccc[nH]3)c2n1 ; C[C@@H]1COCCN1c1cc(C2(S(C)(=O)=O)CC2)n2ncc(-c3ccn[nH]3)c2n1] 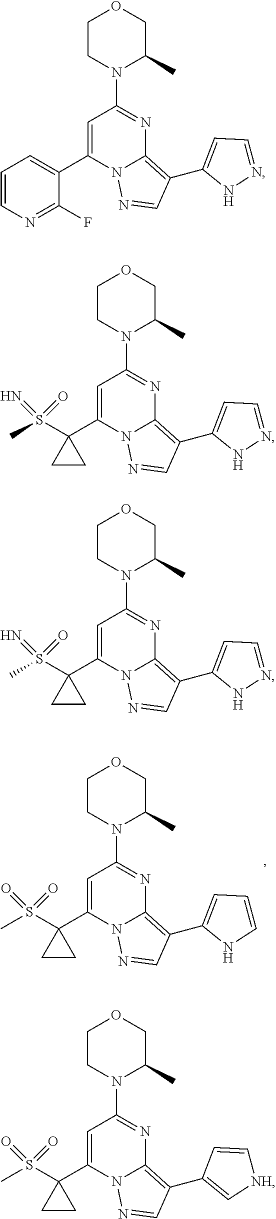 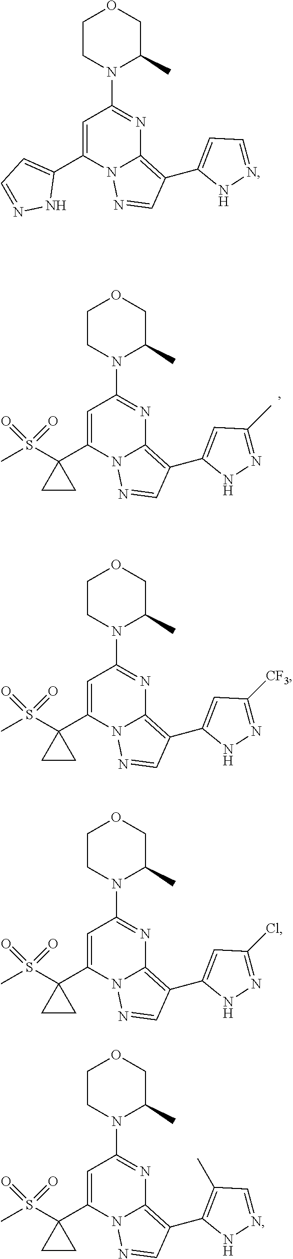 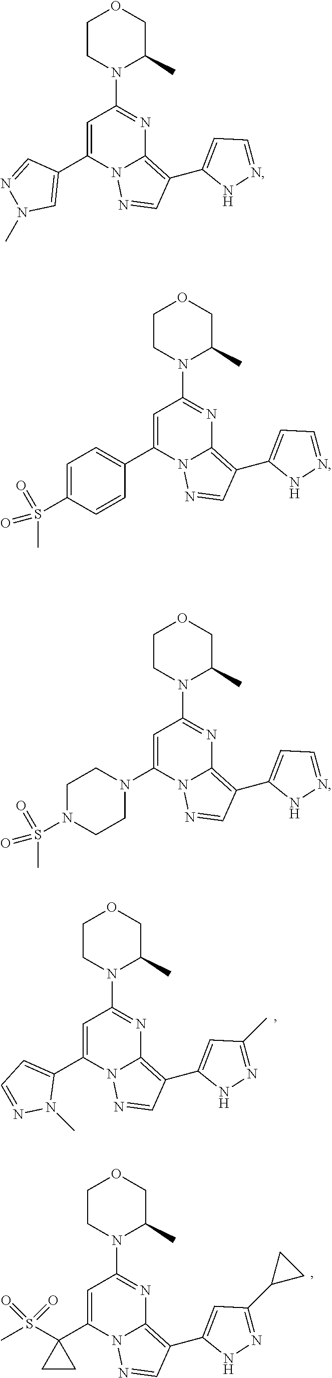 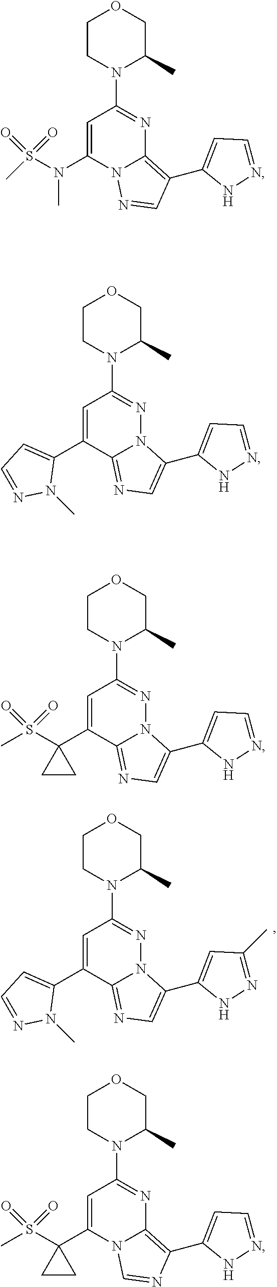 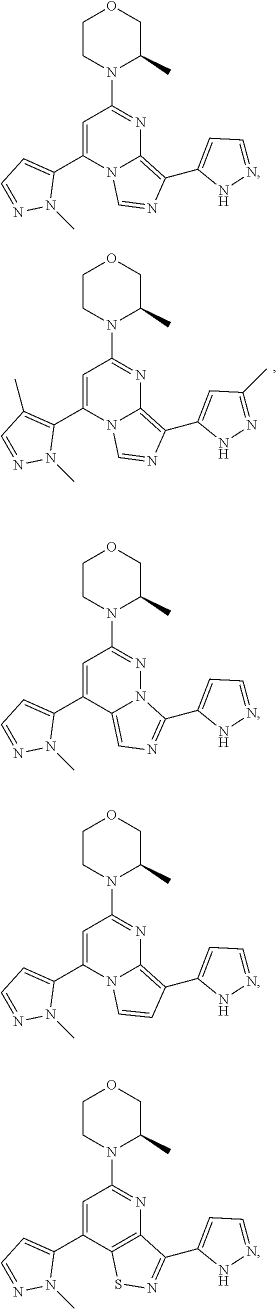 [SMILES: C[C@@H]1COCCN1c1cc(-c2ccnn2C)n2cnc(-c3ccn[nH]3)c2n1 ; Cc1cc(-c2ncn3c(-c4c(C)cnn4C)cc(N4CCOC[C@H]4C)nc23)[nH]n1 ; C[C@@H]1COCCN1c1cc(-c2ccnn2C)c2cnc(-c3ccn[nH]3)n2n1 ; C[C@@H]1COCCN1c1cc(-c2ccnn2C)n2ccc(-c3ccn[nH]3)c2n1 ; C[C@@H]1COCCN1c1cc(-c2ccnn2C)c2snc(-c3ccn[nH]3)c2n1] 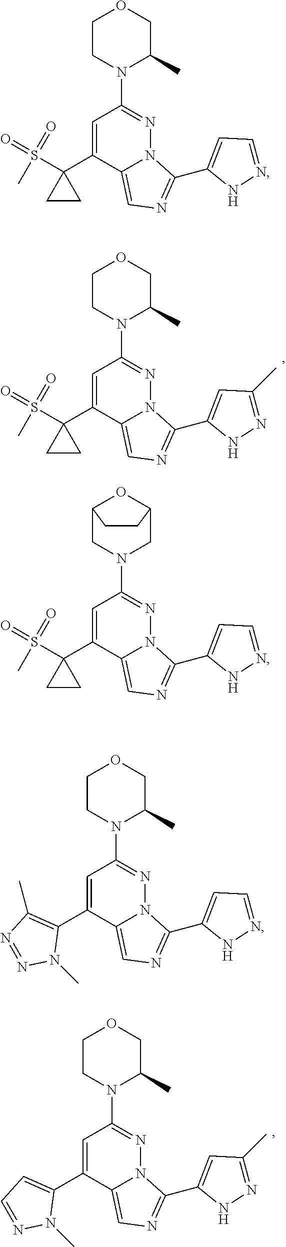 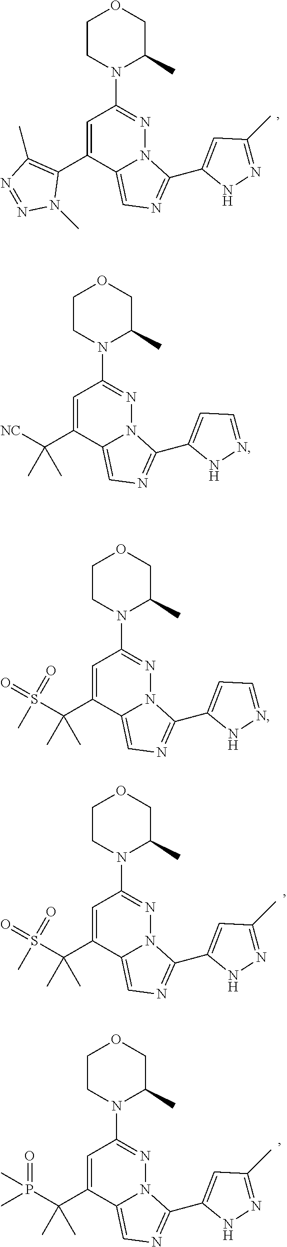 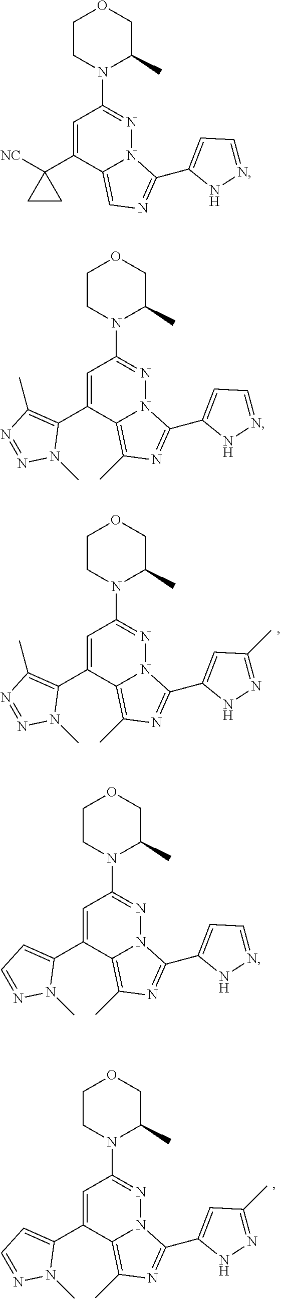 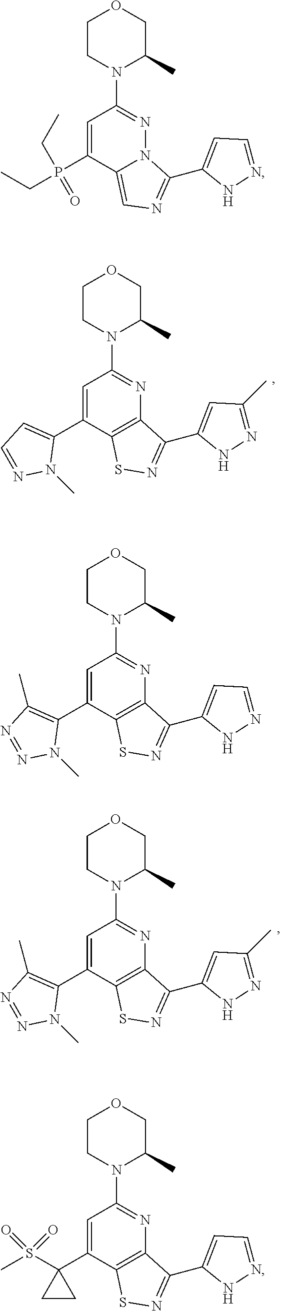 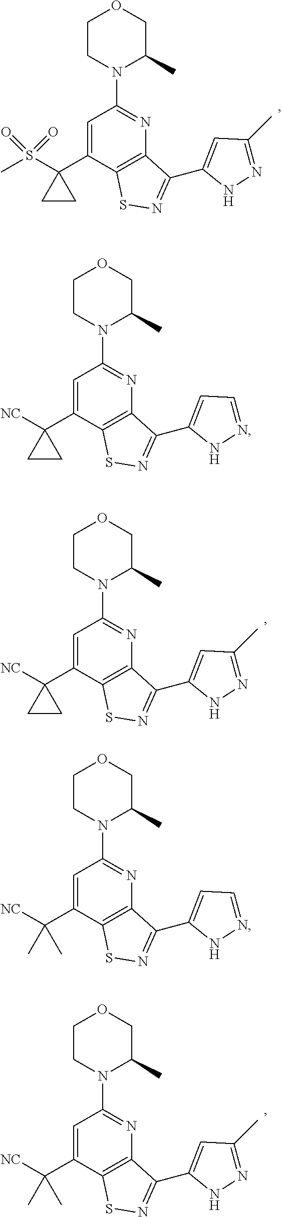 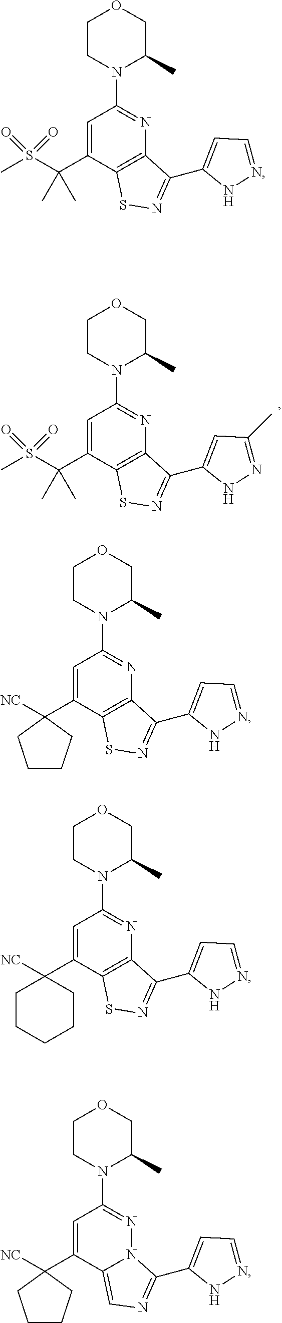 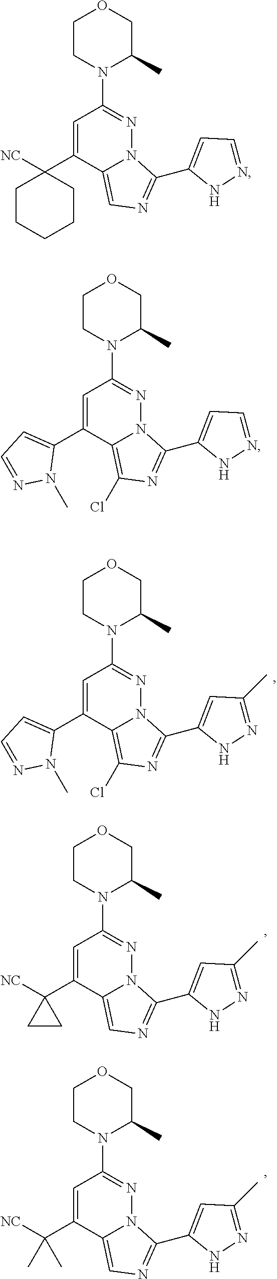 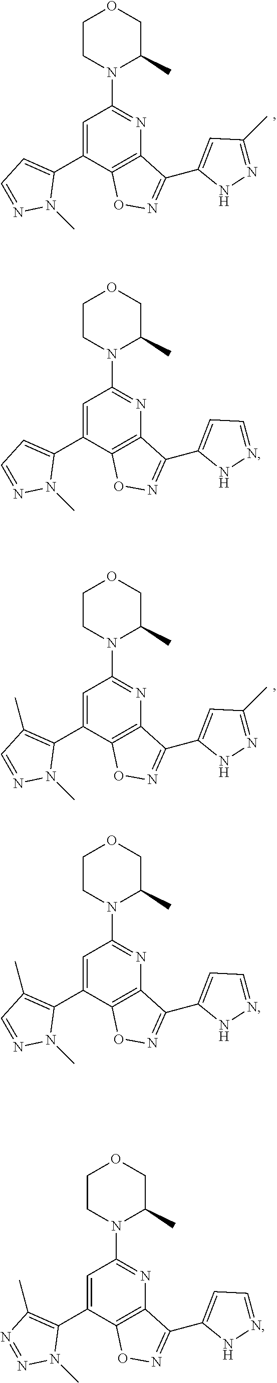 [SMILES: Cc1cc(-c2noc3c(-c4ccnn4C)cc(N4CCOC[C@H]4C)nc23)[nH]n1 ; C[C@@H]1COCCN1c1cc(-c2ccnn2C)c2onc(-c3ccn[nH]3)c2n1 ; Cc1cc(-c2noc3c(-c4c(C)cnn4C)cc(N4CCOC[C@H]4C)nc23)[nH]n1 ; Cc1cnn(C)c1-c1cc(N2CCOC[C@H]2C)nc2c(-c3ccn[nH]3)noc12 ; Cc1nnn(C)c1-c1cc(N2CCOC[C@H]2C)nc2c(-c3ccn[nH]3)noc12] 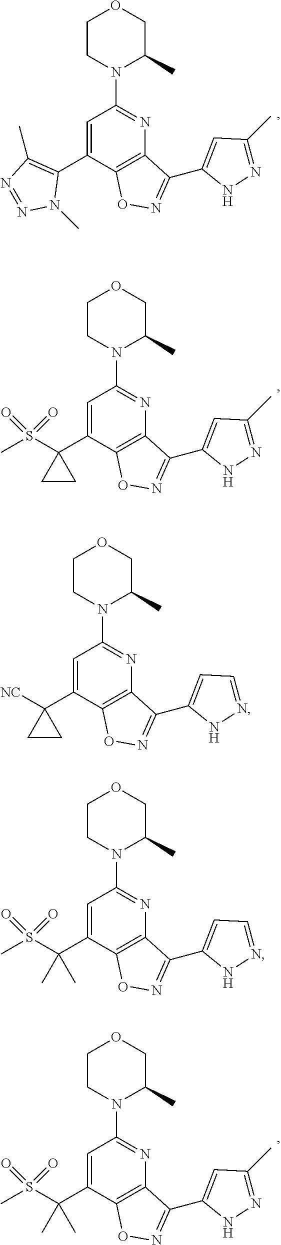 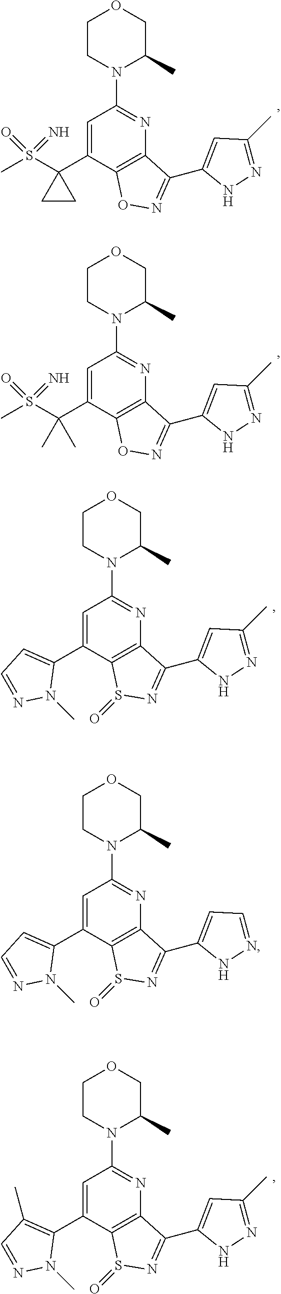 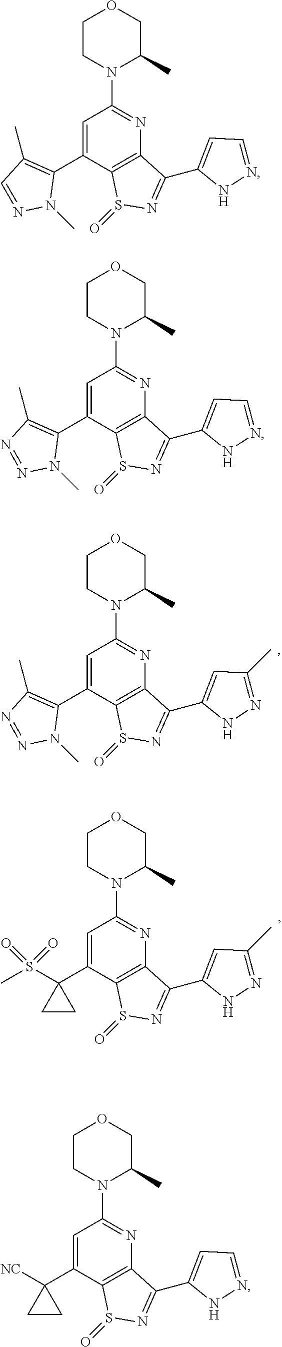 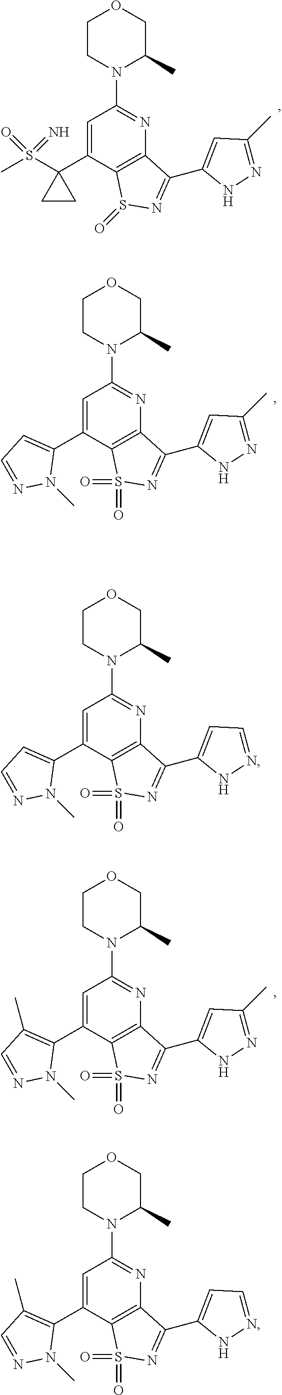 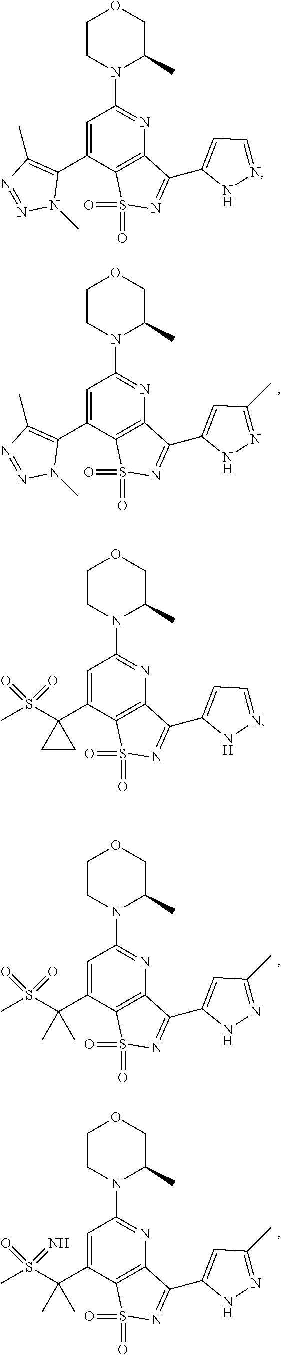 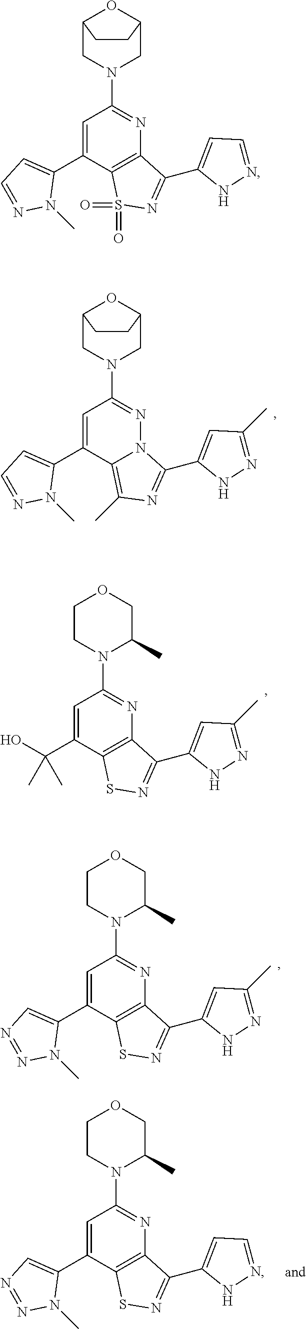 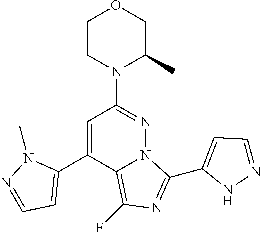 [SMILES: C[C@@H]1COCCN1c1cc(-c2ccnn2C)c2c(F)nc(-c3ccn[nH]3)n2n1]
or a pharmaceutically acceptable salt thereof.

70. A pharmaceutical composition comprising the compound of claim 1 or 2 or a pharmaceutically acceptable salt thereof, and a pharmaceutically acceptable carrier.

71. A method for treating cancer, comprising administering an effective amount of a compound of claim 1 or 2 or a pharmaceutically acceptable salt thereof to a subject in need thereof.

72-73. (canceled)

74. A method for inhibiting ATR kinase in a subject in need thereof, comprising administering an effective amount of a compound of claim 1 or 2 or a pharmaceutically acceptable salt thereof to the subject.
